(19) United States
(12) Patent Application Publication
(10) Pub. No.: US 2025/0320203 A1
(43) Pub. Date: Oct. 16, 2025

(54) 4-(3H-PYRAZOLO[4,3-F]QUINOLIN-7-YL)-N-(2-(DIMETHYLAMINO)ETHYL)BENZAMIDE- OR HYDROXAMIC ACID COMPOUNDS, COMPOSITIONS, AND METHODS OF USE

(71) Applicant: Purdue Research Foundation, West Lafayette, IN (US)

(72) Inventors: Herman O. Sintim, Westfield, IN (US); Neetu Dayal, Agra, Uttar Pradesh (IN); Delmis Hernandez, West Lafayette, IN (US); Allison Kempen, Dubuque, IA (US)

(21) Appl. No.: 18/870,529

(22) PCT Filed: Jun. 2, 2023
(86) PCT No.: PCT/US2023/067822

§ 371 Date: Nov. 29, 2024

Related U.S. Application Data
(60) Provisional application No. 63/348,034, filed on Jun. 2, 2022.

(51) Int. Cl. C07D 471/04; A61K 31/4375; A61K 31/4745; A61K 31/4748; A61K 31/496; A61K 31/5377; A61P 35/02; C07D 471/08; C07D 471/14; C07D 491/147; C07D 495/14; C07D 495/22

(52) U.S. Cl. CPC C07D 471/04; A61K 31/4375; A61K 31/4745; A61K 31/4748; A61K 31/496; A61K 31/5377; A61P 35/02; C07D 471/08; C07D 471/14; C07D 491/147; C07D 495/14; C07D 495/22

(57) ABSTRACT

N-(2-(dimethylamino)ethyl)benzamide- or hydroxamic acid derivative-comprising compounds for the inhibition of certain kinases for use in certain kinase-mediated disease states. Pharmaceutical compositions, conjugates, and methods for treating those kinase-mediated disease states are also provided.

### PRIORITY

This application is related to and claims the priority benefit of U.S. Provisional Patent Application No. 63/348,034 filed Jun. 2, 2022. The content of the aforementioned application is hereby incorporated by reference in its entirety into this disclosure.

### TECHNICAL FIELD

The present invention generally relates to compounds and pharmaceutical compositions comprising 4-(3H-pyrazolo[4,3-f]quinolin-7-yl)-N-(2-(dimethylamino)ethyl)benzamide- or hydroxamic acid potential anti-cancer agents or anti-inflammatory agents for the treatment of diseases mediated by a kinase, such as inflammation and cancer. Methods for treating those diseases are also provided.

### BACKGROUND

This section introduces aspects that may help facilitate a better understanding of the disclosure. Accordingly, these statements are to be read in this light and are not to be understood as admissions about what is or is not prior art.

The cell contains over 500 kinases, which regulate diverse processes such as cell cycle, growth, migration, and immune response. Several deregulated kinases (i.e., kinases that have attained a gain-of-function mutation or are over-expressed) can drive cancer proliferation. Fabbro et al., Ten things you should know about protein kinases: IUPHAR Review 14, British J Pharmacology 172 (11): 2675-2700 (2015). Small molecule inhibitors of cancer-driver kinases (e.g., BCR-ABL1 fusion protein, FMS-like tyrosine kinase-3 (FLT3) internal tandem duplication (FLT3-ITD), mutated or over-expressed anaplastic lymphoma kinase (ALK), epidermal growth factor receptor (EGFR), platelet-derived growth factor receptor (PDGFR), KIT, vascular endothelial growth factor (VEGFR), B-Raf, Bruton tyrosine kinase (BTK), phosphatidylino-4,5-bisphosphate 3-kinase catalytic subunit delta (PI3Kδ), and Erb-B2) have had some clinical success. Fabbro, 25 years of small molecule weight kinase inhibitors: potentials and limitations, Molecular Pharmacology 87 (5): 766-775 (2015). Recent efforts have also been made to target other kinases to arrest cancer growth, such as cell cycle kinases (CDKs) and kinases that target histones, cytoskeleton or other processes that are important for the cell.

However, most of the kinase inhibitors to date are rendered ineffective due to the emergence of resistant clones.

Various mechanisms account for cancer cell resistance to kinase inhibitors, including copy number multiplication, additional kinase mutations (such as secondary mutations that rise in the tyrosine kinase domain of FLT3-ITD kinase, for example) or the activation of alternative kinase pathways and/or downstream targets that can bypass the inhibition of a particular kinase target. Lindblad et al., Aberrant activation of the PI3K/mTOR pathway promotes resistance to sorafenib in AML, Oncogene 35 (39): 5119-5131 (2016).

For example, acute myeloid leukemia (AML) is a devastating disease that affects over 20,000 people in the US each year. About 30% of AML patients harbor mutations in the FLT3 kinase, and for these patients, the prognosis is usually poorer than those without FLT3 mutation. Lin & Chen, Advances in the drug therapies of acute myeloid leukemia (except acute wpromyelocytic leukemia), Drug Design, Development & Therapy 12:1009-1017 (2018). Many FLT3 inhibitors have been developed and trialed in the clinic and a few, including midostaurin, quizartinib, and gilteritinib, are approved in the United States or Japan. While these FLT3 inhibitors have moderately improved the survival of FLT3-harboring AML patients, resistance to the approved FLT3 inhibitors has been observed in the clinic. Specifically, FLT3-F6911 is resistant to midostaurin, while FLT3-F691L is resistant to gilteritinib and quizartinib. Scholl et al., Molecular mechanisms of resistance to FLT3 inhibitors in acute myeloid leukemia: ongoing challenges and future treatments, Cells 9 (11): 2493 (2020). In addition to FLT3 resistant mutations, FLT3-independent resistance pathways, such as NRAS activation or mutations of other kinases, can also emerge after prolonged FLT3i treatment. Alotaibi et al., Patterns of resistance differ in patients with acute myeloid leukemia treated with type I versus type II FLT3 inhibitors, Blood Cancer Discovery 2 (2): 125-134 (2021); Kasi et al., Clonal evolution of AML on novel FMS-like tyrosine kinase-3 (FLT3) inhibitor therapy with evolving actionable targets, Leukemia Research Reports 5:7-10 (2016). In some instances, patients who were initially FLT3-positive become FLT3-negative during treatment; yet the cancer progresses, fueled by alternative pathways. Schmalbrock et al., Clonal evolution of acute myeloid leukemia with FLT3-ITD mutation under treatment with midostaurin, Blood 137 (22): 3093-3104 (2021).

Kinase inhibitors that inhibit a cancer-driver kinase and also downstream targets (both kinase and non-kinase targets, such as histone demethylase) and/or kinases that collaborate with the driver kinase could have enhanced potency and a reduced probability of resistance being generated against that kinase inhibitor. A common challenge, however, with such polypharmacotherapy approaches is promiscuous binding, which can lead to toxicity.

Thus, there is an unmet need for drugs that can deal with FLT3, as well as other oncogenic pathways that may arise during treatment.

### SUMMARY

In certain embodiments, a compound having a structure of formula I, or a pharmaceutically acceptable salt thereof, is provided:

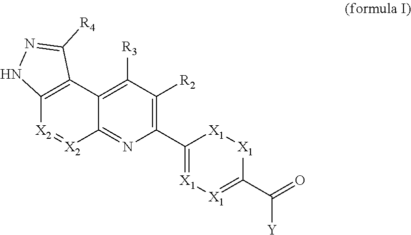

wherein:
X₁ is each independently N, CH, or a C-halogen;
X₂ is each independently N, CH, a C-halogen, C—CHₙ₁Xₘ where n1+m=3 and X is a halogen, C—OCF₃, C—OCHF₂, C—OMe, C—Me, C—Et, C-tBu, C-iPr, C—N-alkyl, C—N-heteroalkyl, C—O-alkyl, or C—O-heteroalkyl;
R₂, R₃, and R₄ are each independently an alkyl, cycloalkyl, heteroalkyl, or heterocycloalkyl; and
Y is

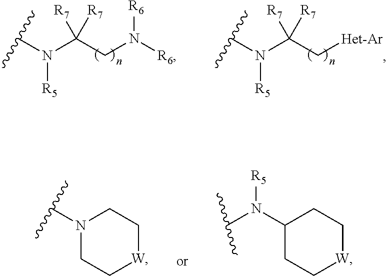

wherein:
n is 1 to 5,
R₅ and R₆ are each independently H, an alkyl, or a heteroalkyl, and R₆ can optionally form a cyclic structure,
R₇ is each independently H or an alkyl,
Het-Ar is a heteroaromatic ring, and
W is NH, NMe, NEt, NCH₂CH₂OH, NCH₂CH₂OMe, O, SO, or SO₂, with the proviso that Y is not NH₂.

In certain embodiments, the at least one of X₁ and/or X₂ is CF, CHF₂, CF₃, or CHF₃, wherein at least one of X₁ and/or X₂ is CF, CHF₂, CF₃, or CHF₃. The compound can have a structure of formula IA:

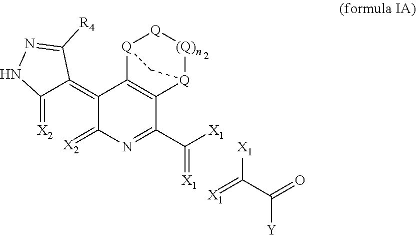

or be a pharmaceutically acceptable salt thereof, wherein:
n2 is 1, 2 or 3;
Q is each independently O, NH, substituted N, CH₂, or substituted C, S, SO, or SO₂; and
each Q, taken together, form a 6-, 7-, or 8-membered ring or bicyclic ring.

One or more of the R₅ and/or R₆ can be Me. In certain embodiments, each R₇ is independently H or Me. In certain embodiments, Y is

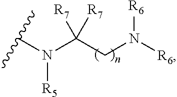

and each R₆, taken together, form a morpholine, piperdine, piperazine, or pyrrolidine. In certain embodiments, Y is

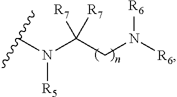

and each R₇, taken together, forms a cyclic structure (e.g., wherein the cyclic structure comprises a cyclopropyl, a cyclobutyl, a cyclopentyl, or a cyclohexyl). In certain embodiments, Y is

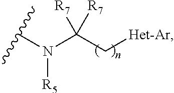

and each R₇, taken together, form a cyclic structure (e.g., wherein the cyclic structure comprises a cyclopropyl, a cyclobutyl, a cyclopentyl, or a cyclohexyl). In certain embodiments, Y is

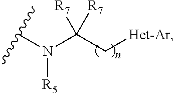

and Het-Ar comprises an imidazole, an oxazole, a pyrazole, a triazole, an isoxazole, an isothiazole, a tetrazole, an oxadiazole, a thiadiazole, a pyrimidine, or a triazine. In certain embodiments, Y is

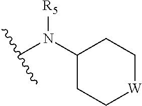

### and R₅ is Me.

The compound can have a structure of formula IB:

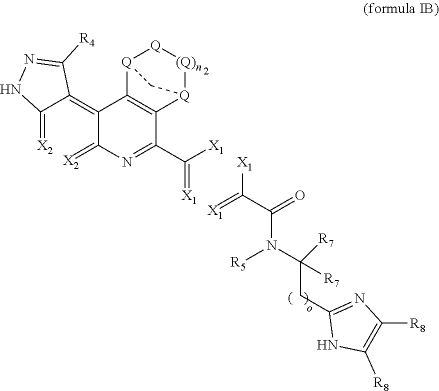

or the compound can be a pharmaceutically acceptable salt thereof, wherein:
is 0-2; and
each R₅ is independently H, an alkyl, or a heteroalkyl. Each R₇, taken together, can form a cyclic structure (e.g., wherein the cyclic structure comprises a cyclopropyl, a cyclobutyl, a cyclopentyl, or a cyclohexyl). In certain embodiments of formula IB, one R₈ is Me, Et or CF₃.

In certain embodiments, the compound has a structure of formula II:

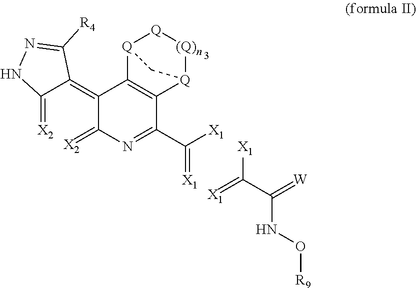

or is a pharmaceutically acceptable salt thereof, wherein:
X₁ is each independently N, CH, or a C-halogen;
X₂ is each independently N, CH, a C-halogen, C—CHₙ₁Xₘ where n1+m=3 and X is a halogen, C—OCF₃, C—OCHF₂, C—OMe, C—Me, C—Et, C-tBu, C-iPr, C—N-alkyl, C—N-heteroalkyl, C—O-alkyl, or C—O-heteroalkyl;
Q is each independently O, NH, substituted N, CH₂, or substituted C, S, SO, or SO₂, and each Q, when taken together, form a 6-, 7-, or 8-membered ring or bicyclic ring;
n3 is 1, 2, or 3;
R₄ is an alkyl, cycloalkyl, heteroalkyl, or heterocycloalkyl;
W is O, NH, or NMe; and
R₉ is an alkyl or a heteroalkyl, with the proviso that R₉ is not H.

In certain embodiments, the compound, or pharmaceutically acceptable salt thereof, has a structure of:

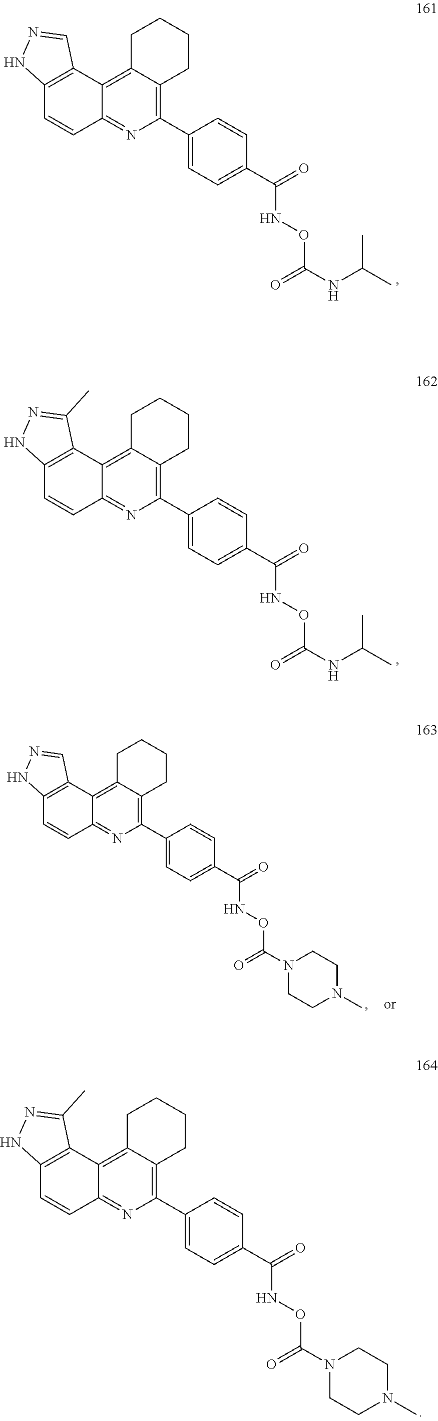 [SMILES: CC(C)NC(=O)ONC(=O)c1ccc(-c2nc3ccc4[nH]ncc4c3c3c2CCCC3)cc1 ; Cc1n[nH]c2ccc3nc(-c4ccc(C(=O)NOC(=O)NC(C)C)cc4)c4c(c3c12)CCCC4 ; CN1CCN(C(=O)ONC(=O)c2ccc(-c3nc4ccc5[nH]ncc5c4c4c3CCCC4)cc2)CC1 ; Cc1n[nH]c2ccc3nc(-c4ccc(C(=O)NOC(=O)N5CCN(C)CC5)cc4)c4c(c3c12)CCCC4]

In certain embodiments, the compound has a structure of

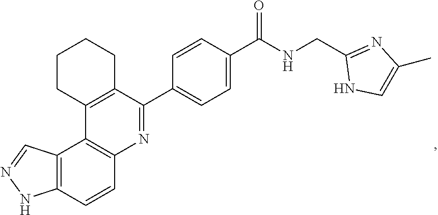 [SMILES: Cc1c[nH]c(CNC(=O)c2ccc(-c3nc4ccc5[nH]ncc5c4c4c3CCCC4)cc2)n1]

or is a pharmaceutically acceptable salt thereof.

The compound can have a structure of:

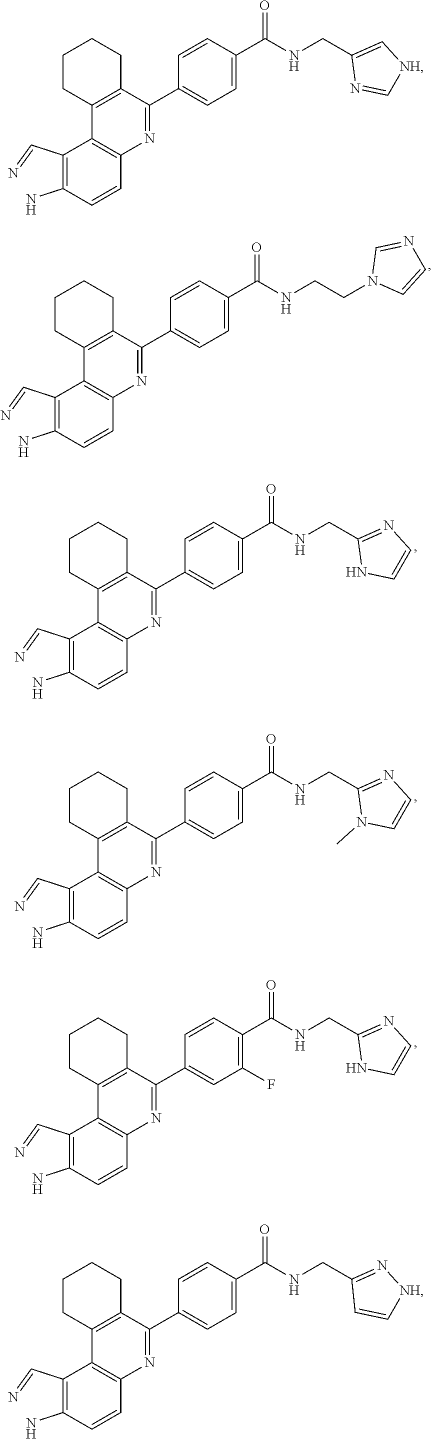 [SMILES: N=Cc1c(N)ccc2nc(-c3ccc(C(=O)NCc4c[nH]cn4)cc3)c3c(c12)CCCC3 ; N=Cc1c(N)ccc2nc(-c3ccc(C(=O)NCCn4ccnc4)cc3)c3c(c12)CCCC3 ; N=Cc1c(N)ccc2nc(-c3ccc(C(=O)NCc4ncc[nH]4)cc3)c3c(c12)CCCC3 ; Cn1ccnc1CNC(=O)c1ccc(-c2nc3ccc(N)c(C=N)c3c3c2CCCC3)cc1 ; N=Cc1c(N)ccc2nc(-c3ccc(C(=O)NCc4ncc[nH]4)c(F)c3)c3c(c12)CCCC3 ; N=Cc1c(N)ccc2nc(-c3ccc(C(=O)NCc4cc[nH]n4)cc3)c3c(c12)CCCC3]

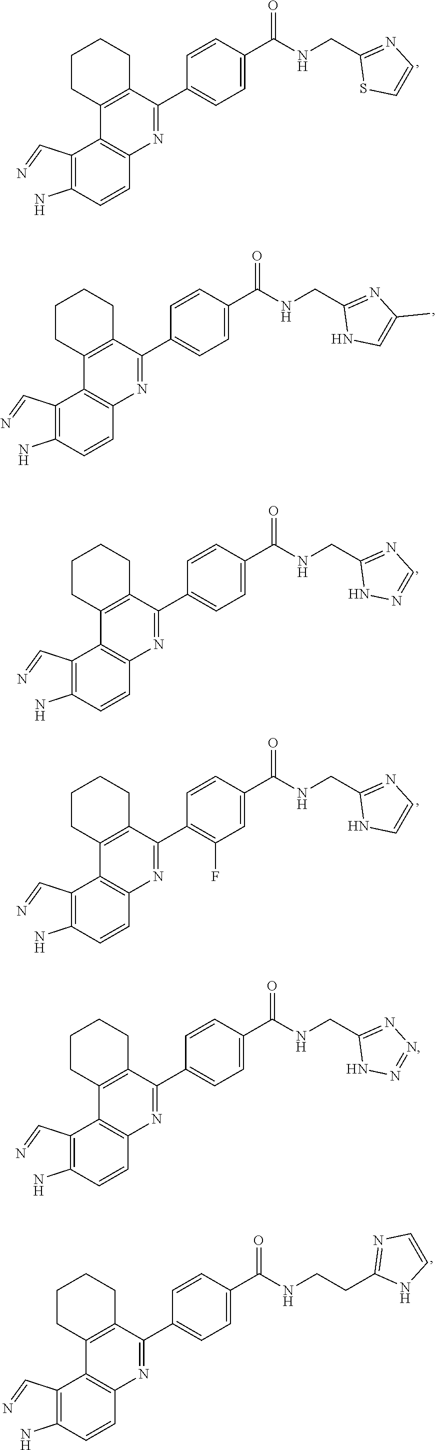 [SMILES: N=Cc1c(N)ccc2nc(-c3ccc(C(=O)NCc4nccs4)cc3)c3c(c12)CCCC3 ; Cc1c[nH]c(CNC(=O)c2ccc(-c3nc4ccc(N)c(C=N)c4c4c3CCCC4)cc2)n1 ; N=Cc1c(N)ccc2nc(-c3ccc(C(=O)NCc4ncn[nH]4)cc3)c3c(c12)CCCC3 ; N=Cc1c(N)ccc2nc(-c3ccc(C(=O)NCc4ncc[nH]4)cc3F)c3c(c12)CCCC3 ; N=Cc1c(N)ccc2nc(-c3ccc(C(=O)NCc4nnn[nH]4)cc3)c3c(c12)CCCC3 ; N=Cc1c(N)ccc2nc(-c3ccc(C(=O)NCCc4ncc[nH]4)cc3)c3c(c12)CCCC3]

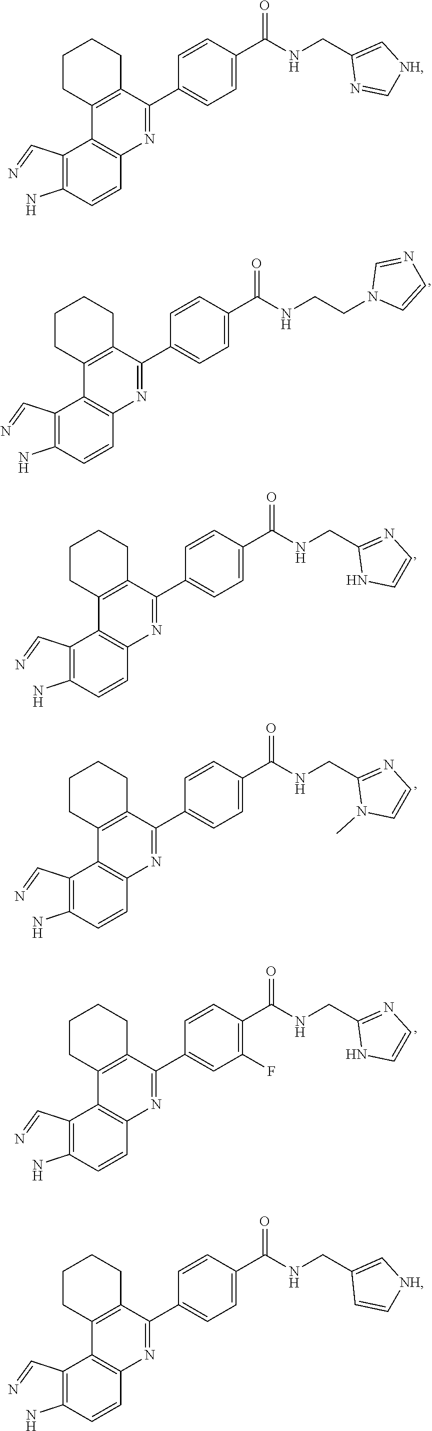 [SMILES: N=Cc1c(N)ccc2nc(-c3ccc(C(=O)NCc4c[nH]cn4)cc3)c3c(c12)CCCC3 ; N=Cc1c(N)ccc2nc(-c3ccc(C(=O)NCCn4ccnc4)cc3)c3c(c12)CCCC3 ; N=Cc1c(N)ccc2nc(-c3ccc(C(=O)NCc4ncc[nH]4)cc3)c3c(c12)CCCC3 ; Cn1ccnc1CNC(=O)c1ccc(-c2nc3ccc(N)c(C=N)c3c3c2CCCC3)cc1 ; N=Cc1c(N)ccc2nc(-c3ccc(C(=O)NCc4ncc[nH]4)c(F)c3)c3c(c12)CCCC3 ; N=Cc1c(N)ccc2nc(-c3ccc(C(=O)NCc4cc[nH]c4)cc3)c3c(c12)CCCC3]

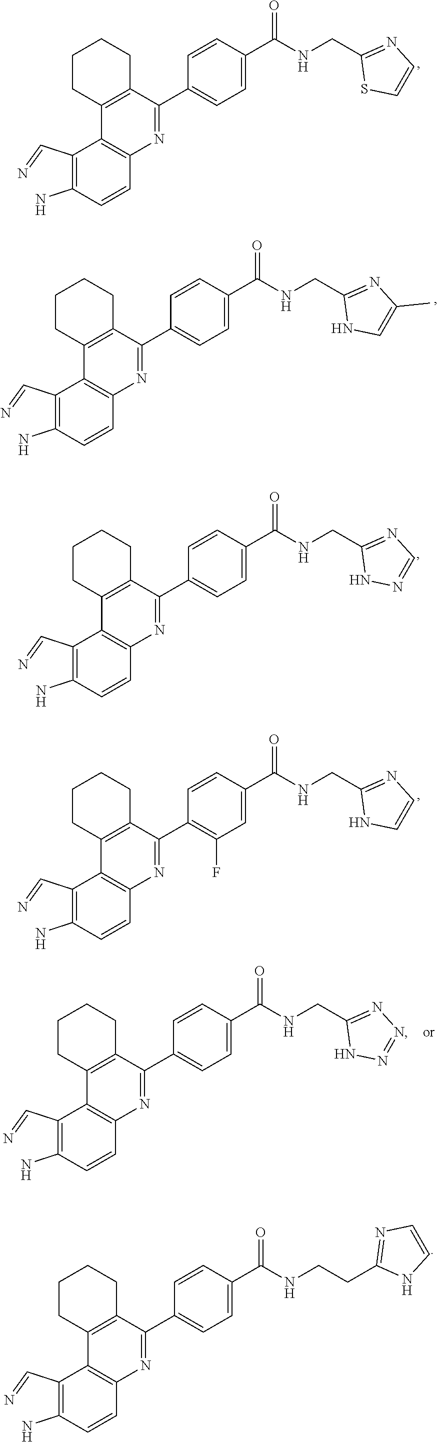 [SMILES: N=Cc1c(N)ccc2nc(-c3ccc(C(=O)NCc4nccs4)cc3)c3c(c12)CCCC3 ; Cc1c[nH]c(CNC(=O)c2ccc(-c3nc4ccc(N)c(C=N)c4c4c3CCCC4)cc2)n1 ; N=Cc1c(N)ccc2nc(-c3ccc(C(=O)NCc4ncn[nH]4)cc3)c3c(c12)CCCC3 ; N=Cc1c(N)ccc2nc(-c3ccc(C(=O)NCc4ncc[nH]4)cc3F)c3c(c12)CCCC3 ; N=Cc1c(N)ccc2nc(-c3ccc(C(=O)NCc4nnn[nH]4)cc3)c3c(c12)CCCC3 ; N=Cc1c(N)ccc2nc(-c3ccc(C(=O)NCCc4ncc[nH]4)cc3)c3c(c12)CCCC3]

The compound can have a structure of:

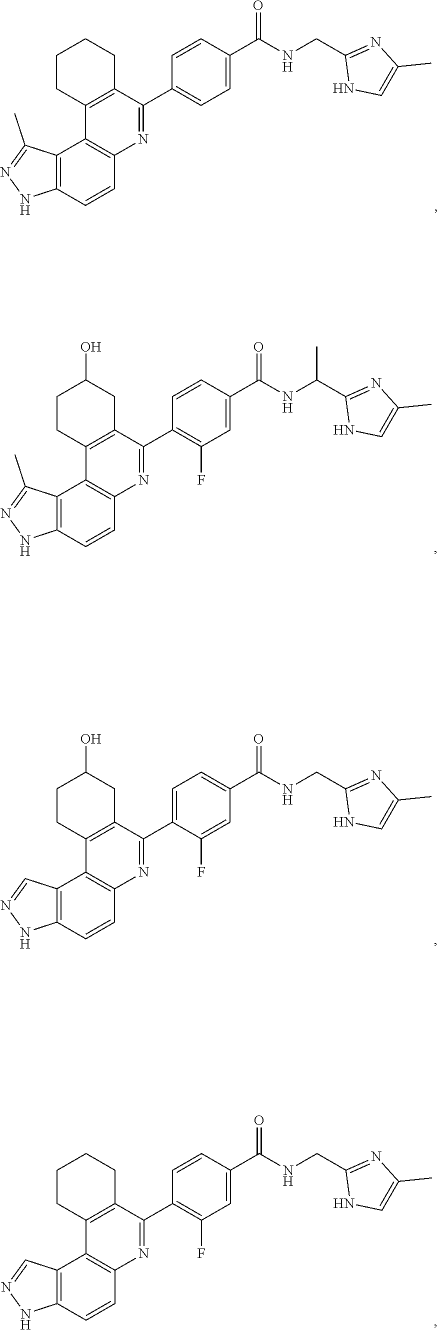 [SMILES: Cc1c[nH]c(CNC(=O)c2ccc(-c3nc4ccc5[nH]nc(C)c5c4c4c3CCCC4)cc2)n1 ; Cc1c[nH]c(C(C)NC(=O)c2ccc(-c3nc4ccc5[nH]nc(C)c5c4c4c3CC(O)CC4)c(F)c2)n1 ; Cc1c[nH]c(CNC(=O)c2ccc(-c3nc4ccc5[nH]ncc5c4c4c3CC(O)CC4)c(F)c2)n1 ; Cc1c[nH]c(CNC(=O)c2ccc(-c3nc4ccc5[nH]ncc5c4c4c3CCCC4)c(F)c2)n1]

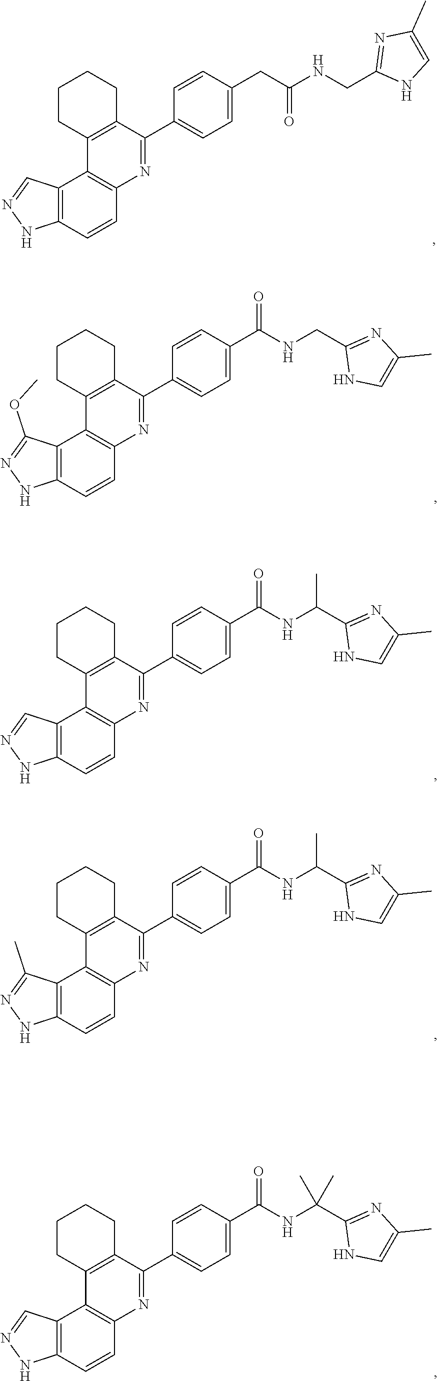 [SMILES: Cc1c[nH]c(CNC(=O)Cc2ccc(-c3nc4ccc5[nH]ncc5c4c4c3CCCC4)cc2)n1 ; COc1n[nH]c2ccc3nc(-c4ccc(C(=O)NCc5nc(C)c[nH]5)cc4)c4c(c3c12)CCCC4 ; Cc1c[nH]c(C(C)NC(=O)c2ccc(-c3nc4ccc5[nH]ncc5c4c4c3CCCC4)cc2)n1 ; Cc1c[nH]c(C(C)NC(=O)c2ccc(-c3nc4ccc5[nH]nc(C)c5c4c4c3CCCC4)cc2)n1 ; Cc1c[nH]c(C(C)(C)NC(=O)c2ccc(-c3nc4ccc5[nH]ncc5c4c4c3CCCC4)cc2)n1]

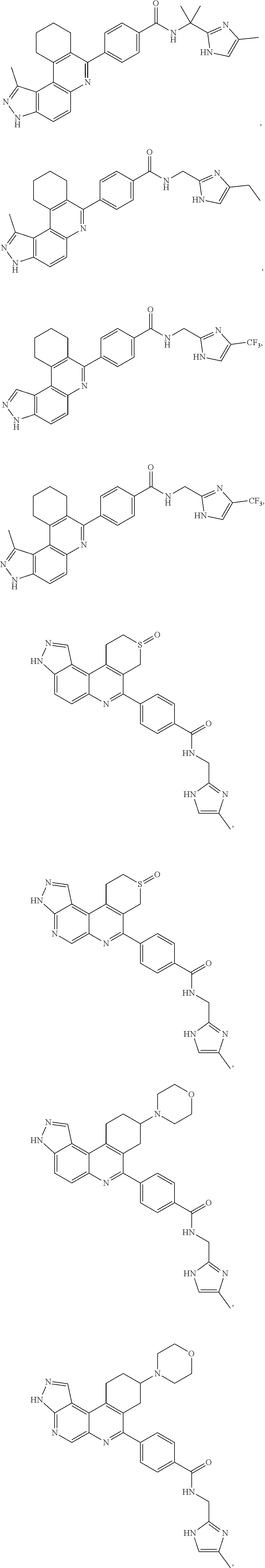

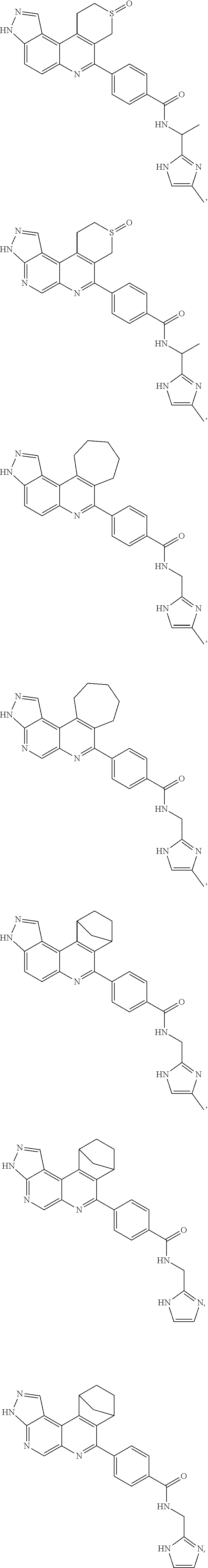

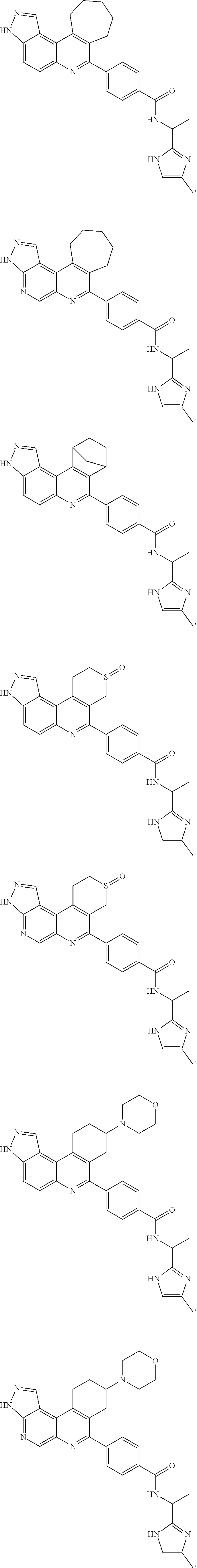

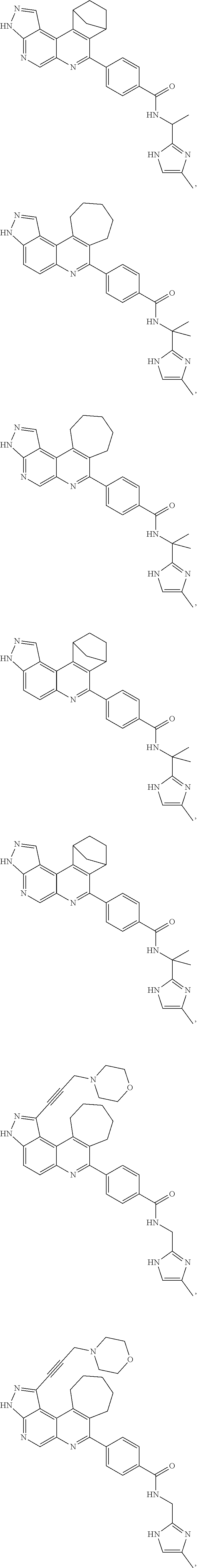

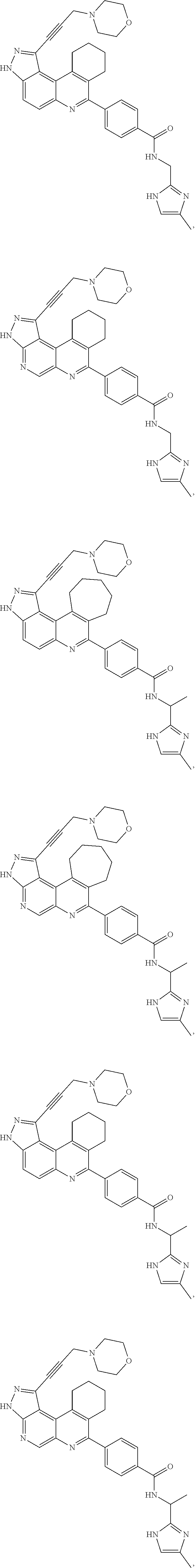 [SMILES: Cc1c[nH]c(CNC(=O)c2ccc(-c3nc4ccc5[nH]nc(C#CCN6CCOCC6)c5c4c4c3CCCC4)cc2)n1 ; Cc1c[nH]c(CNC(=O)c2ccc(-c3nc4cnc5[nH]nc(C#CCN6CCOCC6)c5c4c4c3CCCC4)cc2)n1 ; Cc1c[nH]c(C(C)NC(=O)c2ccc(-c3nc4ccc5[nH]nc(C#CCN6CCOCC6)c5c4c4c3CCCCC4)cc2)n1 ; Cc1c[nH]c(C(C)NC(=O)c2ccc(-c3nc4cnc5[nH]nc(C#CCN6CCOCC6)c5c4c4c3CCCCC4)cc2)n1 ; Cc1c[nH]c(C(C)NC(=O)c2ccc(-c3nc4ccc5[nH]nc(C#CCN6CCOCC6)c5c4c4c3CCCC4)cc2)n1 ; Cc1c[nH]c(C(C)NC(=O)c2ccc(-c3nc4cnc5[nH]nc(C#CCN6CCOCC6)c5c4c4c3CCCC4)cc2)n1]

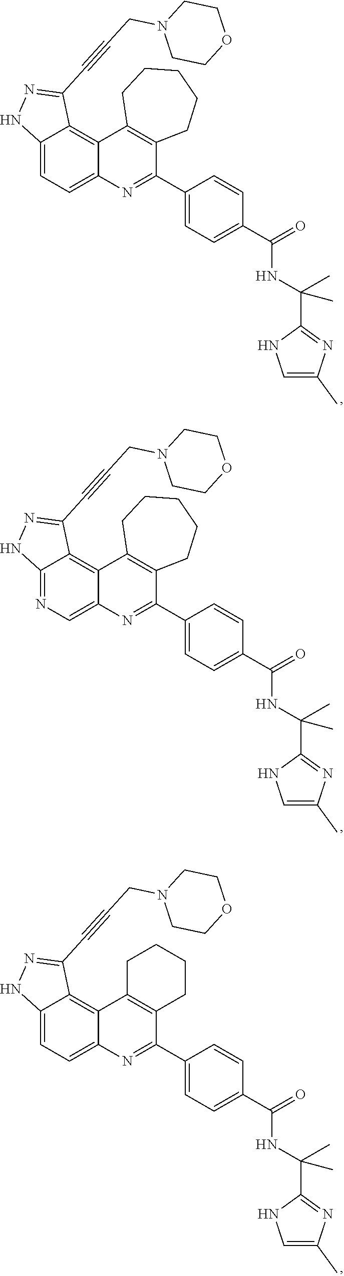 [SMILES: Cc1c[nH]c(C(C)(C)NC(=O)c2ccc(-c3nc4ccc5[nH]nc(C#CCN6CCOCC6)c5c4c4c3CCCCC4)cc2)n1 ; Cc1c[nH]c(C(C)(C)NC(=O)c2ccc(-c3nc4cnc5[nH]nc(C#CCN6CCOCC6)c5c4c4c3CCCCC4)cc2)n1 ; Cc1c[nH]c(C(C)(C)NC(=O)c2ccc(-c3nc4ccc5[nH]nc(C#CCN6CCOCC6)c5c4c4c3CCCC4)cc2)n1]

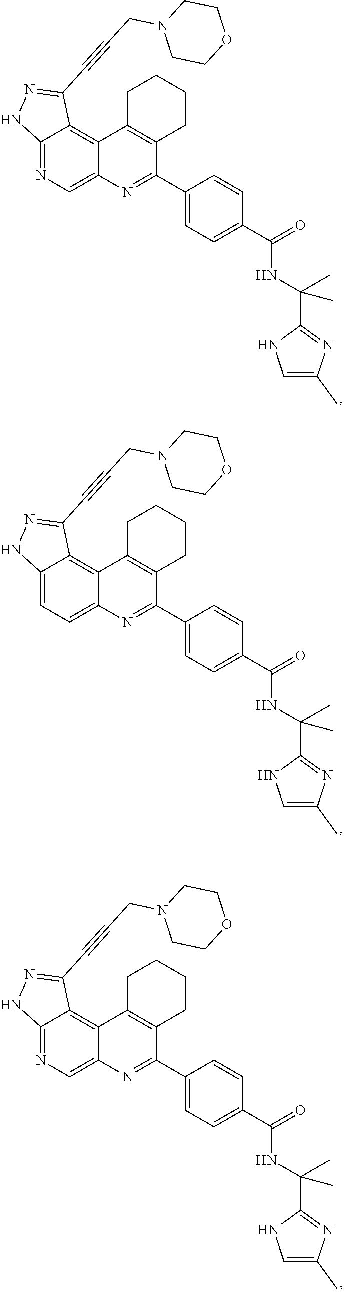 [SMILES: Cc1c[nH]c(C(C)(C)NC(=O)c2ccc(-c3nc4cnc5[nH]nc(C#CCN6CCOCC6)c5c4c4c3CCCC4)cc2)n1 ; Cc1c[nH]c(C(C)(C)NC(=O)c2ccc(-c3nc4ccc5[nH]nc(C#CCN6CCOCC6)c5c4c4c3CCCC4)cc2)n1 ; Cc1c[nH]c(C(C)(C)NC(=O)c2ccc(-c3nc4cnc5[nH]nc(C#CCN6CCOCC6)c5c4c4c3CCCC4)cc2)n1]

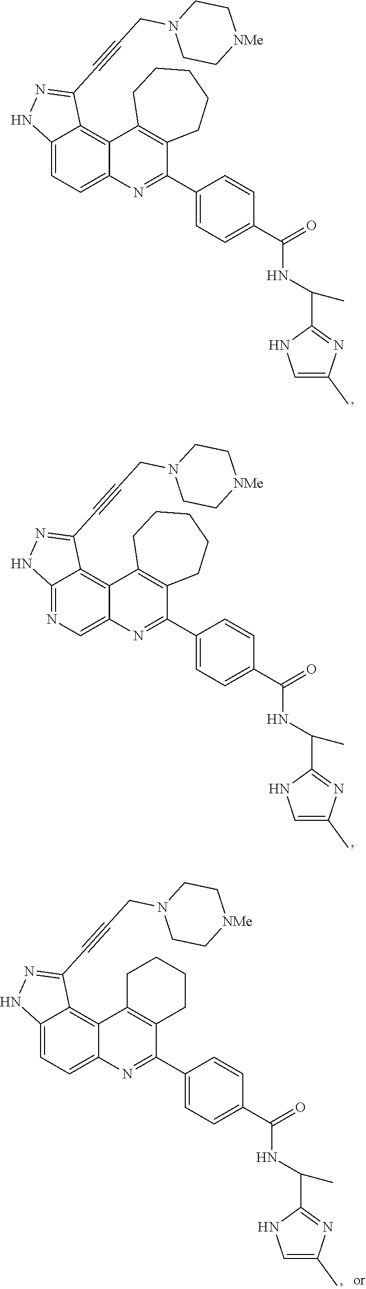 [SMILES: Cc1c[nH]c(C(C)NC(=O)c2ccc(-c3nc4ccc5[nH]nc(C#CCN6CCN(C)CC6)c5c4c4c3CCCCC4)cc2)n1 ; Cc1c[nH]c(C(C)NC(=O)c2ccc(-c3nc4cnc5[nH]nc(C#CCN6CCN(C)CC6)c5c4c4c3CCCCC4)cc2)n1 ; Cc1c[nH]c(C(C)NC(=O)c2ccc(-c3nc4ccc5[nH]nc(C#CCN6CCN(C)CC6)c5c4c4c3CCCC4)cc2)n1]

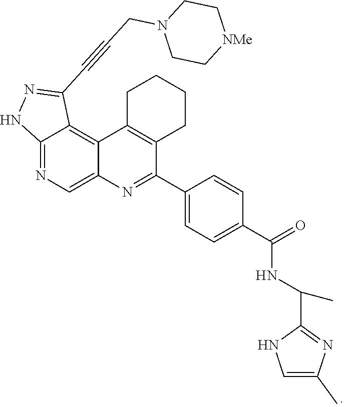 [SMILES: Cc1c[nH]c(C(C)NC(=O)c2ccc(-c3nc4cnc5[nH]nc(C#CCN6CCN(C)CC6)c5c4c4c3CCCC4)cc2)n1]

The compound can have a structure of:

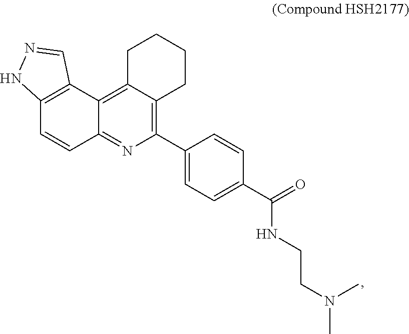 [SMILES: CN(C)CCNC(=O)c1ccc(-c2nc3ccc4[nH]ncc4c3c3c2CCCC3)cc1]

or can be a pharmaceutically acceptable salt thereof.

The compound can have a structure of:

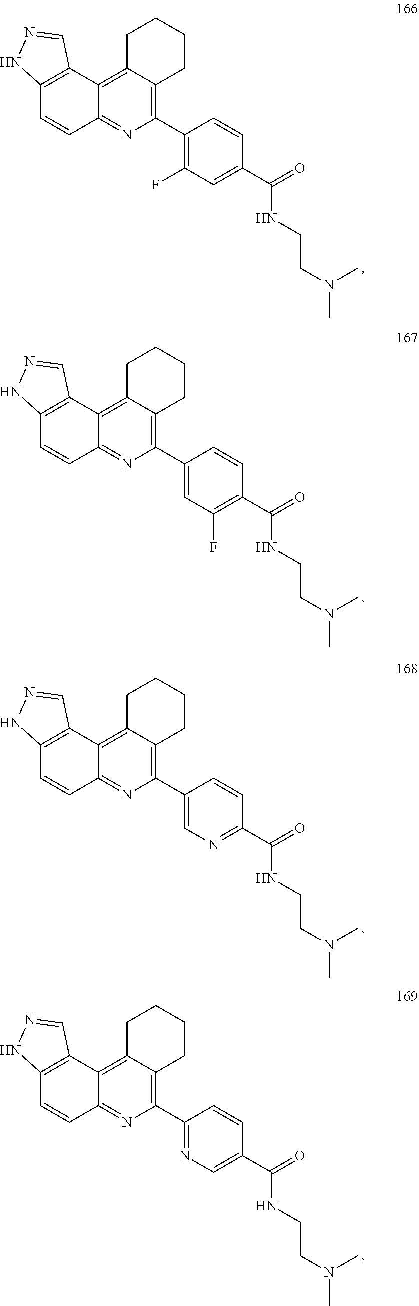 [SMILES: CN(C)CCNC(=O)c1ccc(-c2nc3ccc4[nH]ncc4c3c3c2CCCC3)c(F)c1 ; CN(C)CCNC(=O)c1ccc(-c2nc3ccc4[nH]ncc4c3c3c2CCCC3)cc1F ; CN(C)CCNC(=O)c1ccc(-c2nc3ccc4[nH]ncc4c3c3c2CCCC3)cn1 ; CN(C)CCNC(=O)c1ccc(-c2nc3ccc4[nH]ncc4c3c3c2CCCC3)nc1]

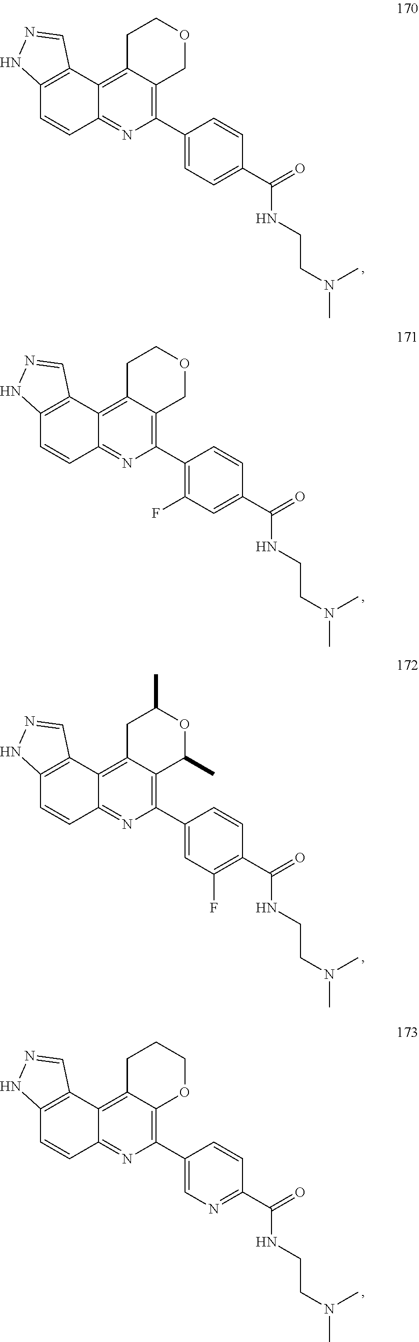 [SMILES: CN(C)CCNC(=O)c1ccc(-c2nc3ccc4[nH]ncc4c3c3c2COCC3)cc1 ; CN(C)CCNC(=O)c1ccc(-c2nc3ccc4[nH]ncc4c3c3c2COCC3)c(F)c1 ; C[C@@H]1Cc2c(c(-c3ccc(C(=O)NCCN(C)C)c(F)c3)nc3ccc4[nH]ncc4c23)[C@H](C)O1 ; CN(C)CCNC(=O)c1ccc(-c2nc3ccc4[nH]ncc4c3c3c2OCCC3)cn1]

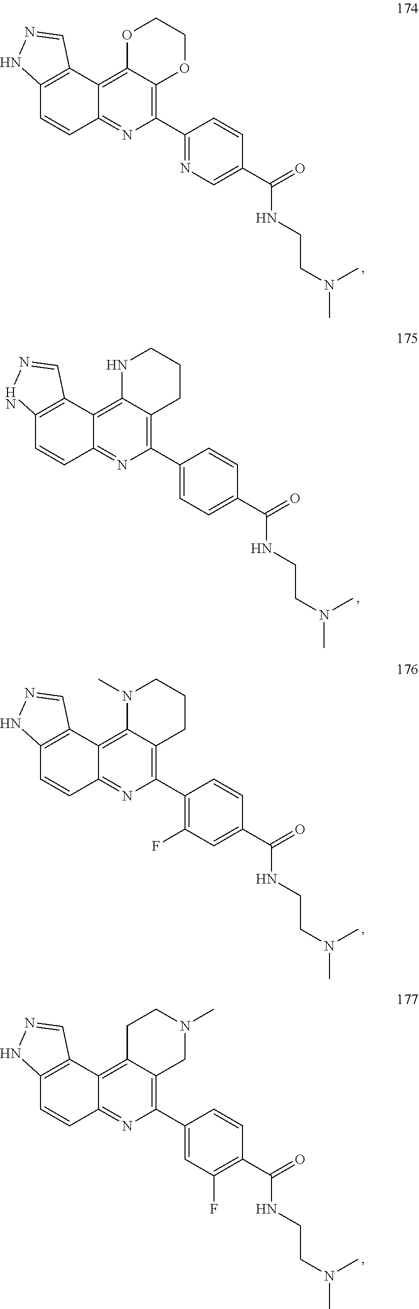 [SMILES: CN(C)CCNC(=O)c1ccc(-c2nc3ccc4[nH]ncc4c3c3c2OCCO3)nc1 ; CN(C)CCNC(=O)c1ccc(-c2nc3ccc4[nH]ncc4c3c3c2CCCN3)cc1 ; CN(C)CCNC(=O)c1ccc(-c2nc3ccc4[nH]ncc4c3c3c2CCCN3C)c(F)c1 ; CN(C)CCNC(=O)c1ccc(-c2nc3ccc4[nH]ncc4c3c3c2CN(C)CC3)cc1F]

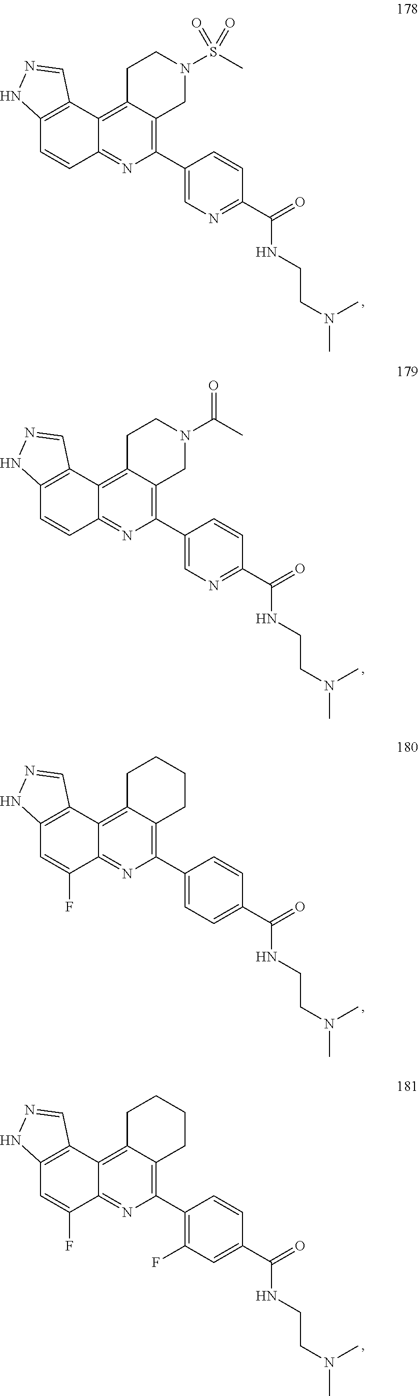 [SMILES: CN(C)CCNC(=O)c1ccc(-c2nc3ccc4[nH]ncc4c3c3c2CN(S(C)(=O)=O)CC3)cn1 ; CC(=O)N1CCc2c(c(-c3ccc(C(=O)NCCN(C)C)nc3)nc3ccc4[nH]ncc4c23)C1 ; CN(C)CCNC(=O)c1ccc(-c2nc3c(F)cc4[nH]ncc4c3c3c2CCCC3)cc1 ; CN(C)CCNC(=O)c1ccc(-c2nc3c(F)cc4[nH]ncc4c3c3c2CCCC3)c(F)c1]

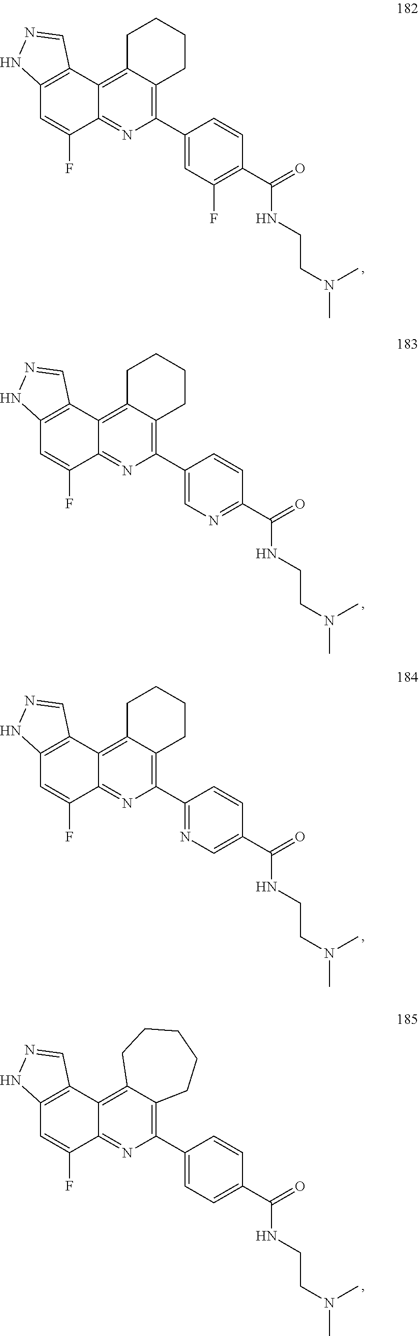 [SMILES: CN(C)CCNC(=O)c1ccc(-c2nc3c(F)cc4[nH]ncc4c3c3c2CCCC3)cc1F ; CN(C)CCNC(=O)c1ccc(-c2nc3c(F)cc4[nH]ncc4c3c3c2CCCC3)cn1 ; CN(C)CCNC(=O)c1ccc(-c2nc3c(F)cc4[nH]ncc4c3c3c2CCCC3)nc1 ; CN(C)CCNC(=O)c1ccc(-c2nc3c(F)cc4[nH]ncc4c3c3c2CCCCC3)cc1]

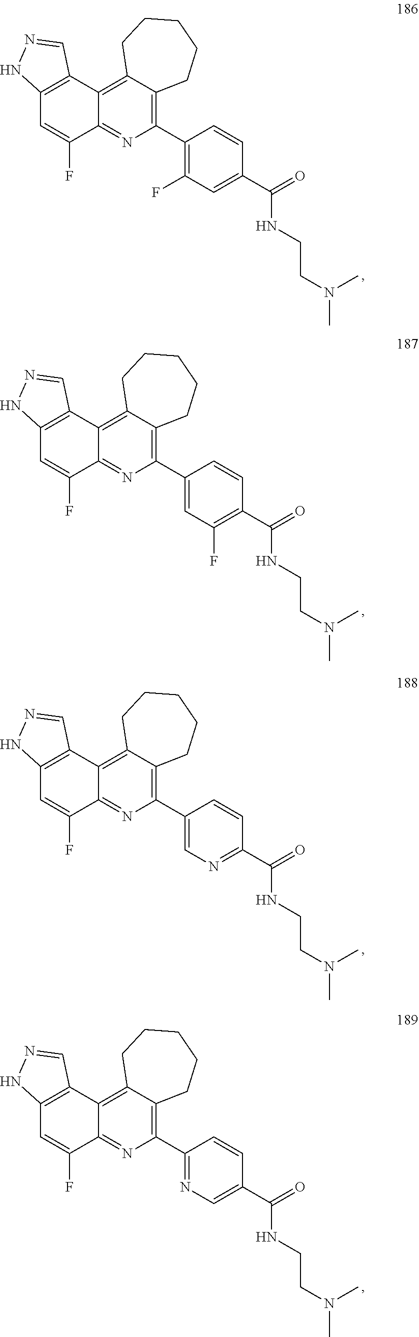 [SMILES: CN(C)CCNC(=O)c1ccc(-c2nc3c(F)cc4[nH]ncc4c3c3c2CCCCC3)c(F)c1 ; CN(C)CCNC(=O)c1ccc(-c2nc3c(F)cc4[nH]ncc4c3c3c2CCCCC3)cc1F ; CN(C)CCNC(=O)c1ccc(-c2nc3c(F)cc4[nH]ncc4c3c3c2CCCCC3)cn1 ; CN(C)CCNC(=O)c1ccc(-c2nc3c(F)cc4[nH]ncc4c3c3c2CCCCC3)nc1]

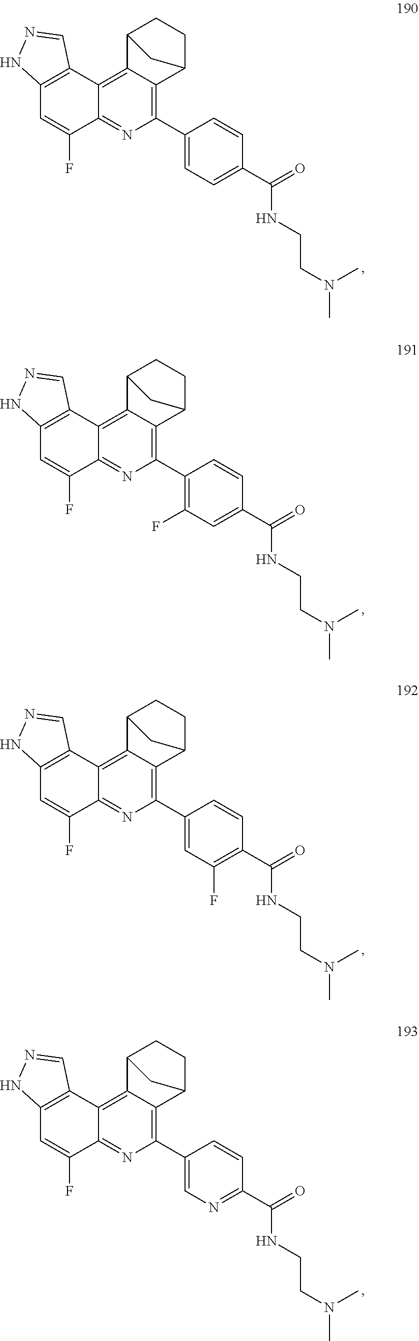 [SMILES: CN(C)CCNC(=O)c1ccc(-c2nc3c(F)cc4[nH]ncc4c3c3c2C2CCC3C2)cc1 ; CN(C)CCNC(=O)c1ccc(-c2nc3c(F)cc4[nH]ncc4c3c3c2C2CCC3C2)c(F)c1 ; CN(C)CCNC(=O)c1ccc(-c2nc3c(F)cc4[nH]ncc4c3c3c2C2CCC3C2)cc1F ; CN(C)CCNC(=O)c1ccc(-c2nc3c(F)cc4[nH]ncc4c3c3c2C2CCC3C2)cn1]

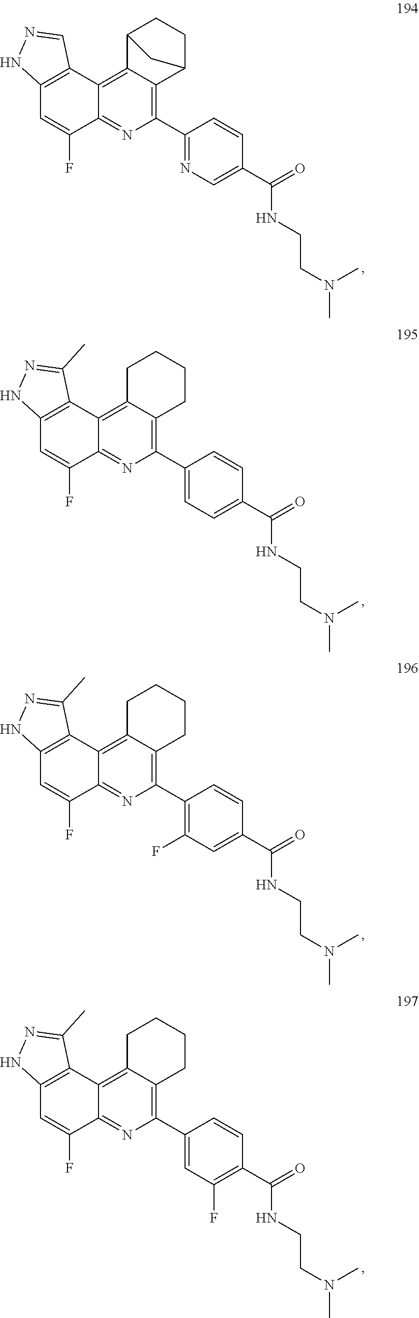 [SMILES: CN(C)CCNC(=O)c1ccc(-c2nc3c(F)cc4[nH]ncc4c3c3c2C2CCC3C2)nc1 ; Cc1n[nH]c2cc(F)c3nc(-c4ccc(C(=O)NCCN(C)C)cc4)c4c(c3c12)CCCC4 ; Cc1n[nH]c2cc(F)c3nc(-c4ccc(C(=O)NCCN(C)C)cc4F)c4c(c3c12)CCCC4 ; Cc1n[nH]c2cc(F)c3nc(-c4ccc(C(=O)NCCN(C)C)c(F)c4)c4c(c3c12)CCCC4]

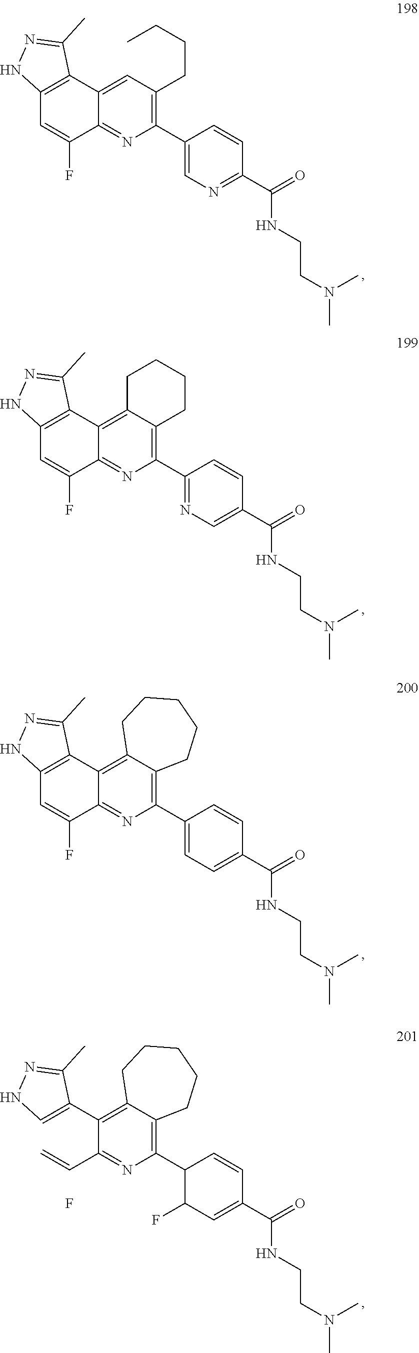

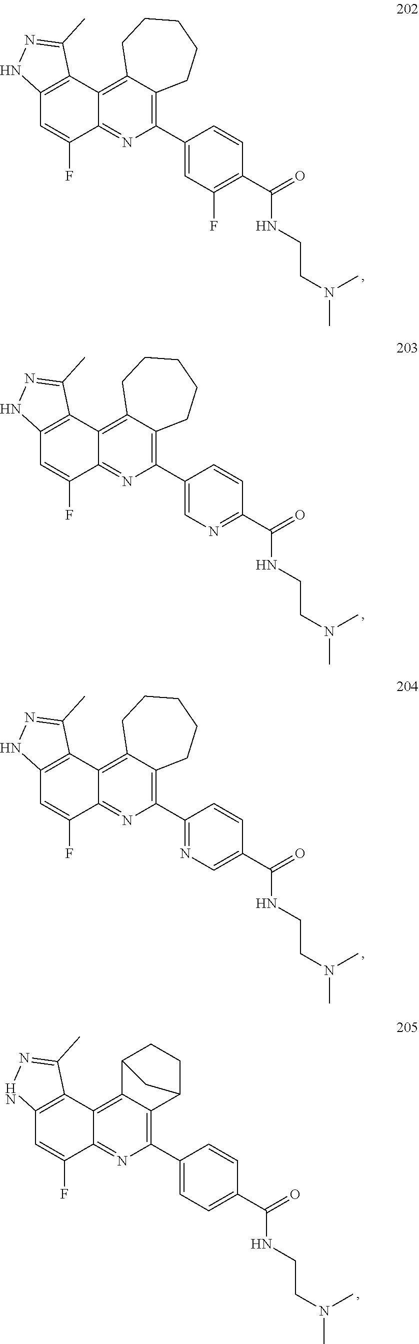 [SMILES: Cc1n[nH]c2cc(F)c3nc(-c4ccc(C(=O)NCCN(C)C)c(F)c4)c4c(c3c12)CCCCC4 ; Cc1n[nH]c2cc(F)c3nc(-c4ccc(C(=O)NCCN(C)C)nc4)c4c(c3c12)CCCCC4 ; Cc1n[nH]c2cc(F)c3nc(-c4ccc(C(=O)NCCN(C)C)cn4)c4c(c3c12)CCCCC4 ; Cc1n[nH]c2cc(F)c3nc(-c4ccc(C(=O)NCCN(C)C)cc4)c4c(c3c12)C1CCC4C1]

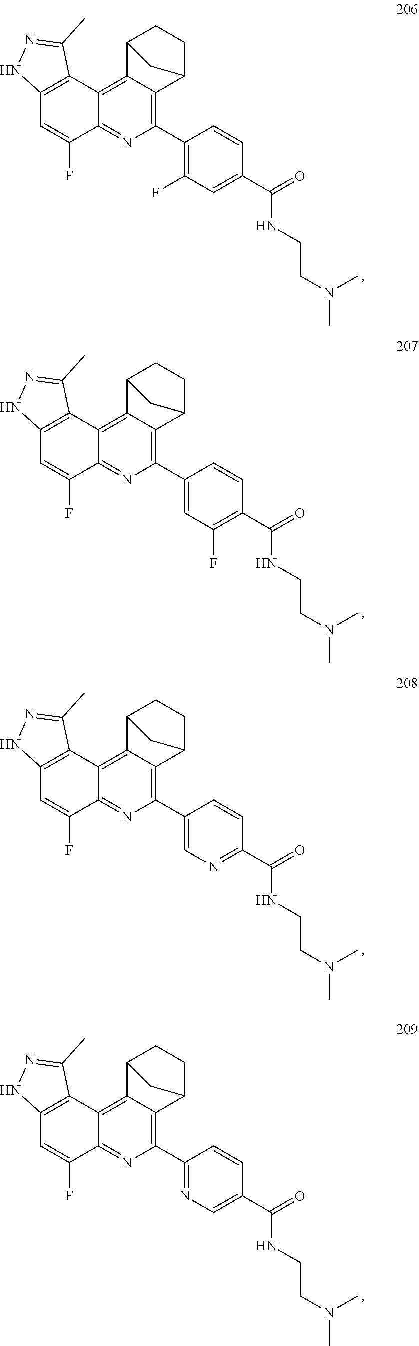 [SMILES: Cc1n[nH]c2cc(F)c3nc(-c4ccc(C(=O)NCCN(C)C)cc4F)c4c(c3c12)C1CCC4C1 ; Cc1n[nH]c2cc(F)c3nc(-c4ccc(C(=O)NCCN(C)C)c(F)c4)c4c(c3c12)C1CCC4C1 ; Cc1n[nH]c2cc(F)c3nc(-c4ccc(C(=O)NCCN(C)C)nc4)c4c(c3c12)C1CCC4C1 ; Cc1n[nH]c2cc(F)c3nc(-c4ccc(C(=O)NCCN(C)C)cn4)c4c(c3c12)C1CCC4C1]

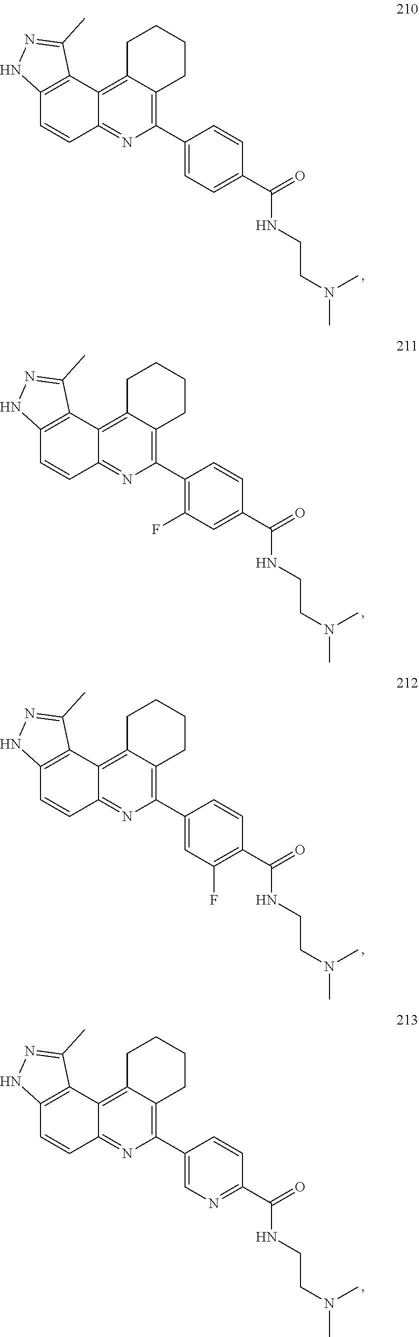 [SMILES: Cc1n[nH]c2ccc3nc(-c4ccc(C(=O)NCCN(C)C)cc4)c4c(c3c12)CCCC4 ; Cc1n[nH]c2ccc3nc(-c4ccc(C(=O)NCCN(C)C)cc4F)c4c(c3c12)CCCC4 ; Cc1n[nH]c2ccc3nc(-c4ccc(C(=O)NCCN(C)C)c(F)c4)c4c(c3c12)CCCC4 ; Cc1n[nH]c2ccc3nc(-c4ccc(C(=O)NCCN(C)C)nc4)c4c(c3c12)CCCC4]

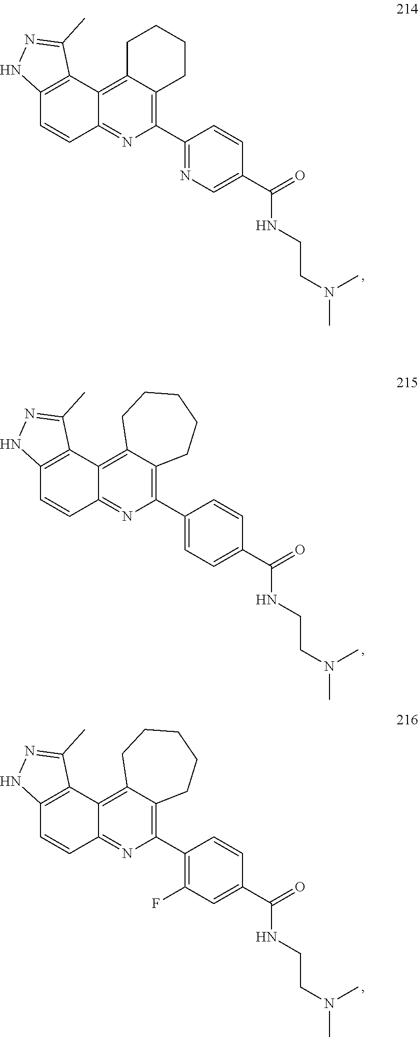 [SMILES: Cc1n[nH]c2ccc3nc(-c4ccc(C(=O)NCCN(C)C)cn4)c4c(c3c12)CCCC4 ; Cc1n[nH]c2ccc3nc(-c4ccc(C(=O)NCCN(C)C)cc4)c4c(c3c12)CCCCC4 ; Cc1n[nH]c2ccc3nc(-c4ccc(C(=O)NCCN(C)C)cc4F)c4c(c3c12)CCCCC4]

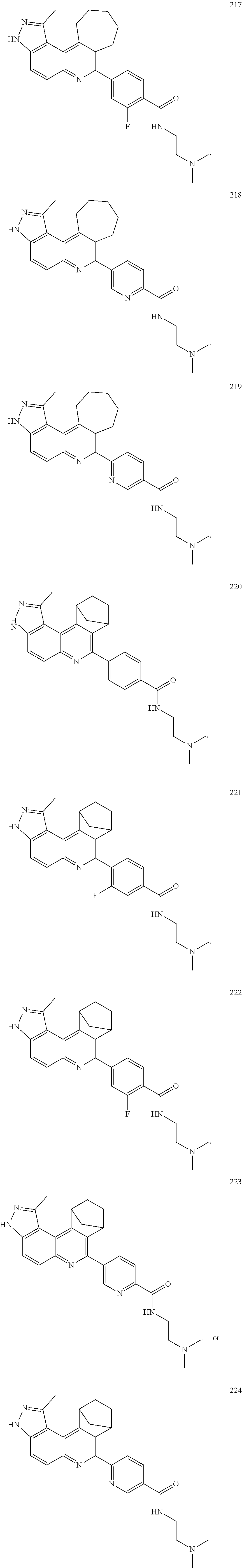

The compound can have a structure of:

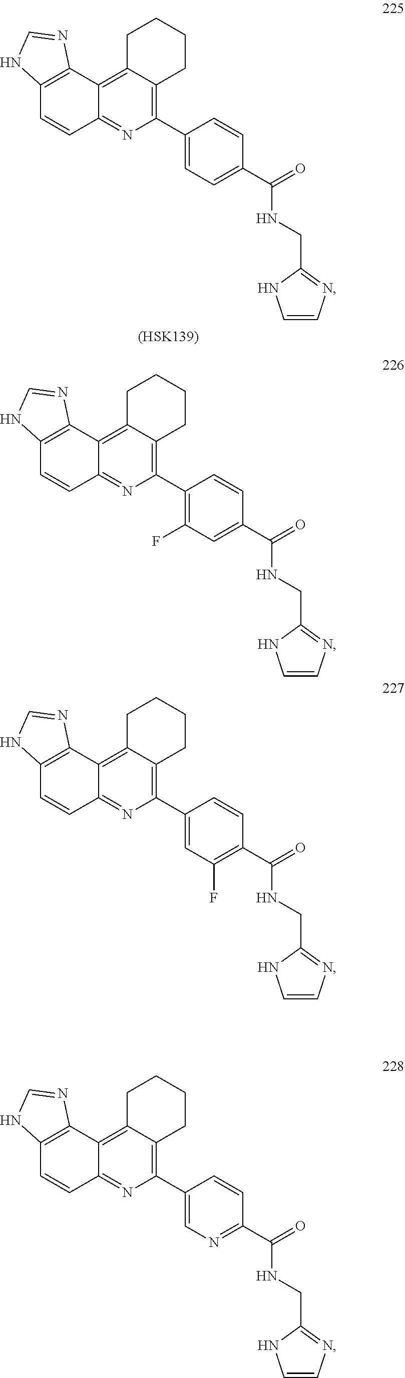 [SMILES: O=C(NCc1ncc[nH]1)c1ccc(-c2nc3ccc4[nH]cnc4c3c3c2CCCC3)c(F)c1 ; O=C(NCc1ncc[nH]1)c1ccc(-c2nc3ccc4[nH]cnc4c3c3c2CCCC3)cc1 ; "(HSK139)" ; O=C(NCc1ncc[nH]1)c1ccc(-c2nc3ccc4[nH]cnc4c3c3c2CCCC3)cc1F ; O=C(NCc1ncc[nH]1)c1ccc(-c2nc3ccc4[nH]cnc4c3c3c2CCCC3)cn1]

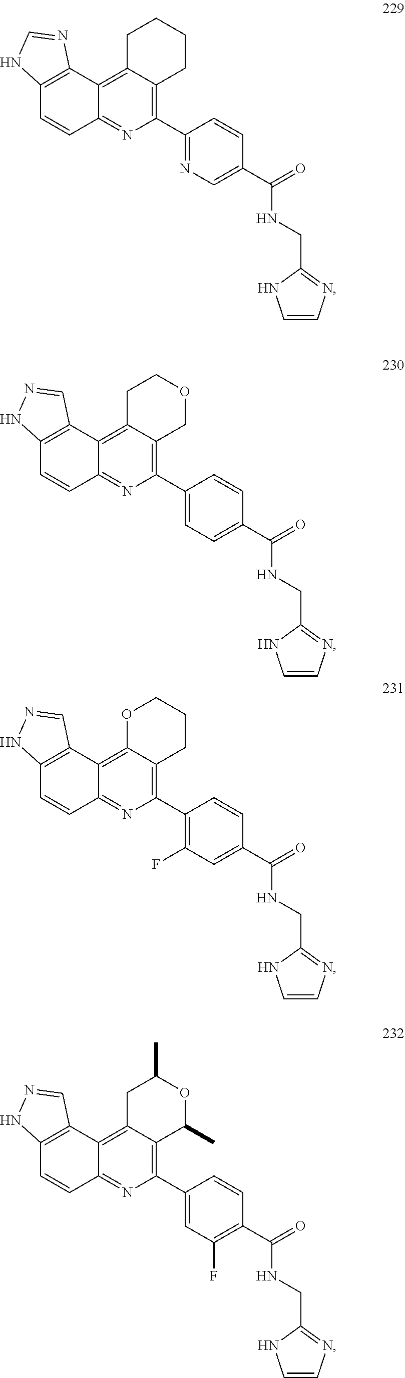 [SMILES: O=C(NCc1ncc[nH]1)c1ccc(-c2nc3ccc4[nH]cnc4c3c3c2CCCC3)nc1 ; O=C(NCc1ncc[nH]1)c1ccc(-c2nc3ccc4[nH]ncc4c3c3c2COCC3)cc1 ; O=C(NCc1ncc[nH]1)c1ccc(-c2nc3ccc4[nH]ncc4c3c3c2CCCO3)c(F)c1 ; C[C@@H]1Cc2c(c(-c3ccc(C(=O)NCc4ncc[nH]4)c(F)c3)nc3ccc4[nH]ncc4c23)[C@H](C)O1]

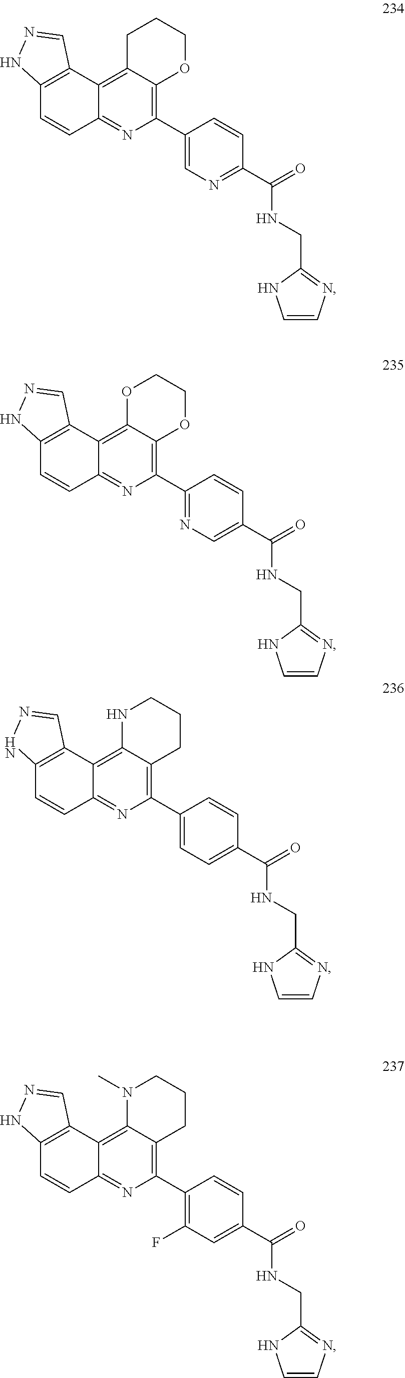 [SMILES: O=C(NCc1ncc[nH]1)c1ccc(-c2nc3ccc4[nH]ncc4c3c3c2OCCC3)cn1 ; O=C(NCc1ncc[nH]1)c1ccc(-c2nc3ccc4[nH]ncc4c3c3c2OCCO3)nc1 ; O=C(NCc1ncc[nH]1)c1ccc(-c2nc3ccc4[nH]ncc4c3c3c2CCCN3)cc1 ; CN1CCCc2c(-c3ccc(C(=O)NCc4ncc[nH]4)cc3F)nc3ccc4[nH]ncc4c3c21]

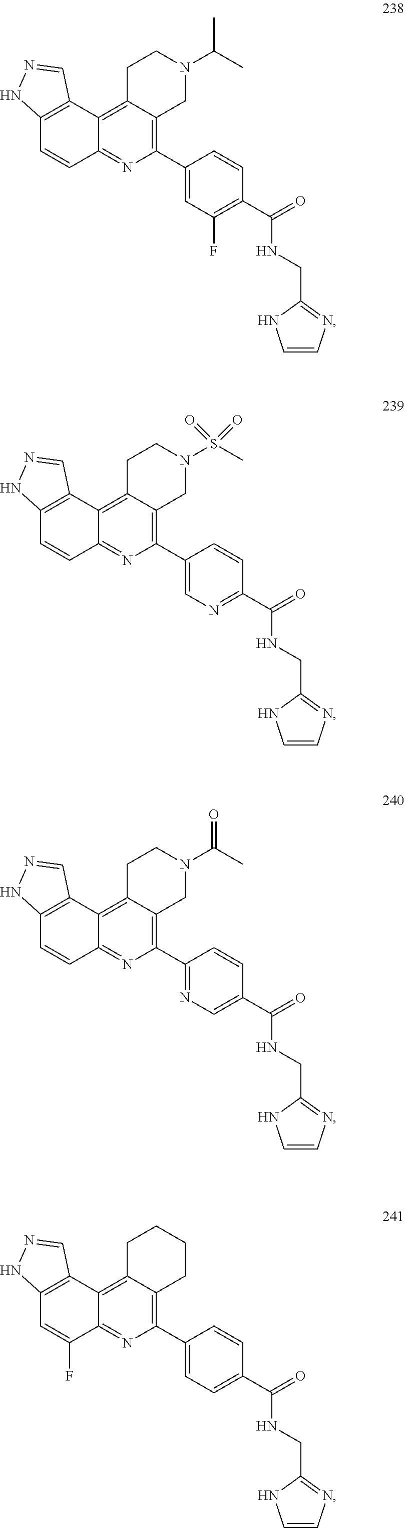 [SMILES: CC(C)N1CCc2c(c(-c3ccc(C(=O)NCc4ncc[nH]4)c(F)c3)nc3ccc4[nH]ncc4c23)C1 ; CS(=O)(=O)N1CCc2c(c(-c3ccc(C(=O)NCc4ncc[nH]4)nc3)nc3ccc4[nH]ncc4c23)C1 ; CC(=O)N1CCc2c(c(-c3ccc(C(=O)NCc4ncc[nH]4)cn3)nc3ccc4[nH]ncc4c23)C1 ; O=C(NCc1ncc[nH]1)c1ccc(-c2nc3c(F)cc4[nH]ncc4c3c3c2CCCC3)cc1]

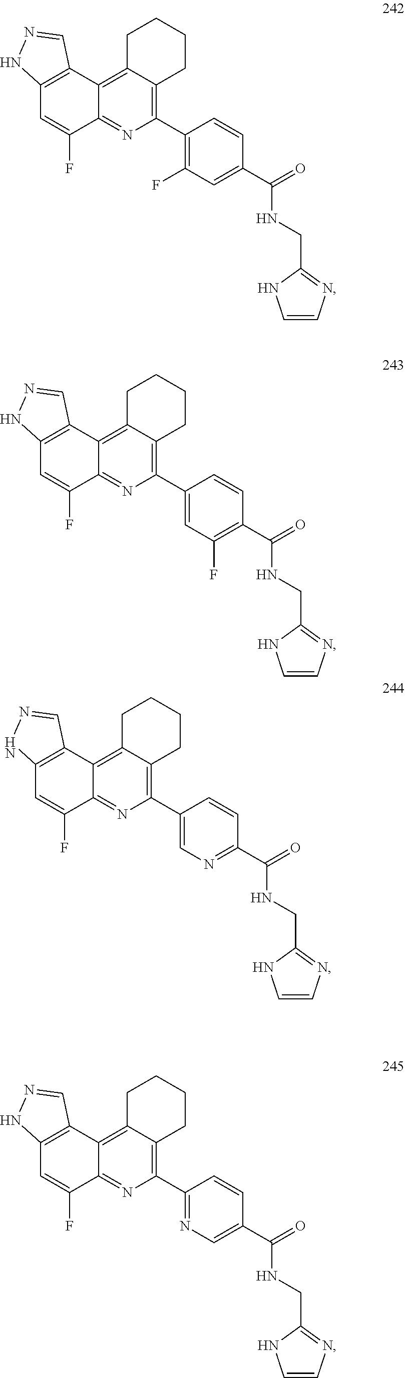 [SMILES: O=C(NCc1ncc[nH]1)c1ccc(-c2nc3c(F)cc4[nH]ncc4c3c3c2CCCC3)c(F)c1 ; O=C(NCc1ncc[nH]1)c1ccc(-c2nc3c(F)cc4[nH]ncc4c3c3c2CCCC3)cc1F ; O=C(NCc1ncc[nH]1)c1ccc(-c2nc3c(F)cc4[nH]ncc4c3c3c2CCCC3)cn1 ; O=C(NCc1ncc[nH]1)c1ccc(-c2nc3c(F)cc4[nH]ncc4c3c3c2CCCC3)nc1]

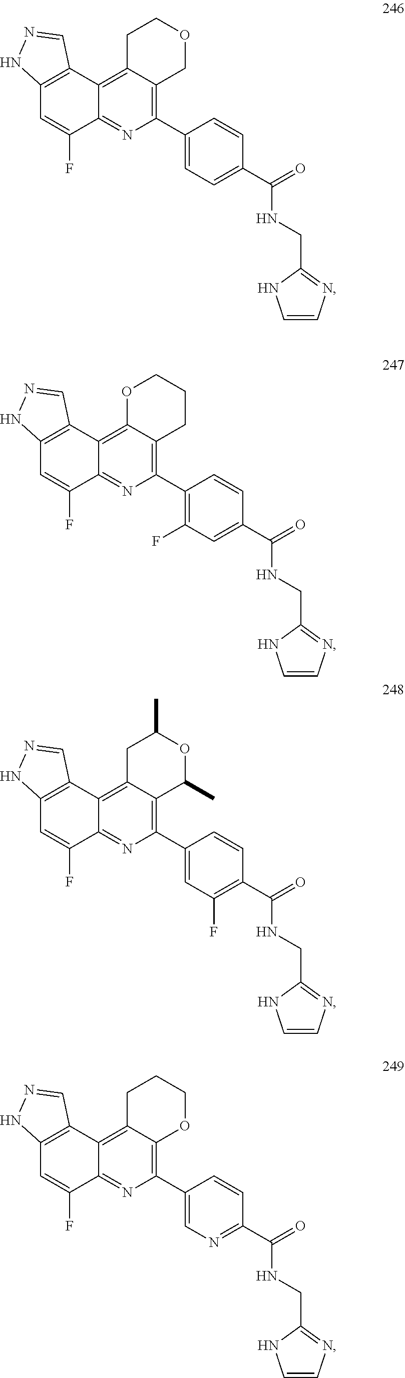 [SMILES: O=C(NCc1ncc[nH]1)c1ccc(-c2nc3c(F)cc4[nH]ncc4c3c3c2COCC3)cc1 ; O=C(NCc1ncc[nH]1)c1ccc(-c2nc3c(F)cc4[nH]ncc4c3c3c2CCCO3)c(F)c1 ; C[C@@H]1Cc2c(c(-c3ccc(C(=O)NCc4ncc[nH]4)c(F)c3)nc3c(F)cc4[nH]ncc4c23)[C@H](C)O1 ; O=C(NCc1ncc[nH]1)c1ccc(-c2nc3c(F)cc4[nH]ncc4c3c3c2OCCC3)cn1]

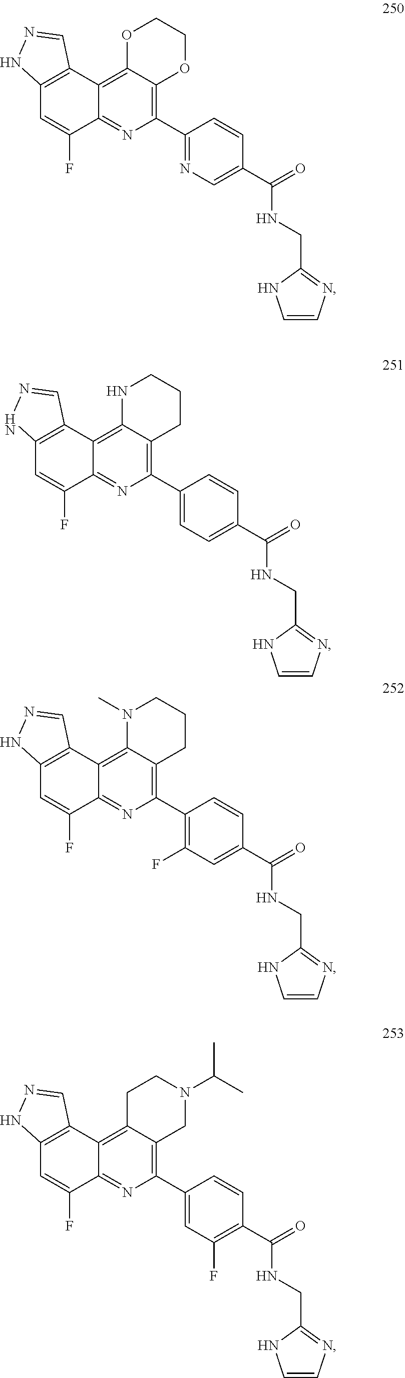 [SMILES: O=C(NCc1ncc[nH]1)c1ccc(-c2nc3c(F)cc4[nH]ncc4c3c3c2OCCO3)nc1 ; O=C(NCc1ncc[nH]1)c1ccc(-c2nc3c(F)cc4[nH]ncc4c3c3c2CCCN3)cc1 ; CN1CCCc2c(-c3ccc(C(=O)NCc4ncc[nH]4)cc3F)nc3c(F)cc4[nH]ncc4c3c21 ; CC(C)N1CCc2c(c(-c3ccc(C(=O)NCc4ncc[nH]4)c(F)c3)nc3c(F)cc4[nH]ncc4c23)C1]

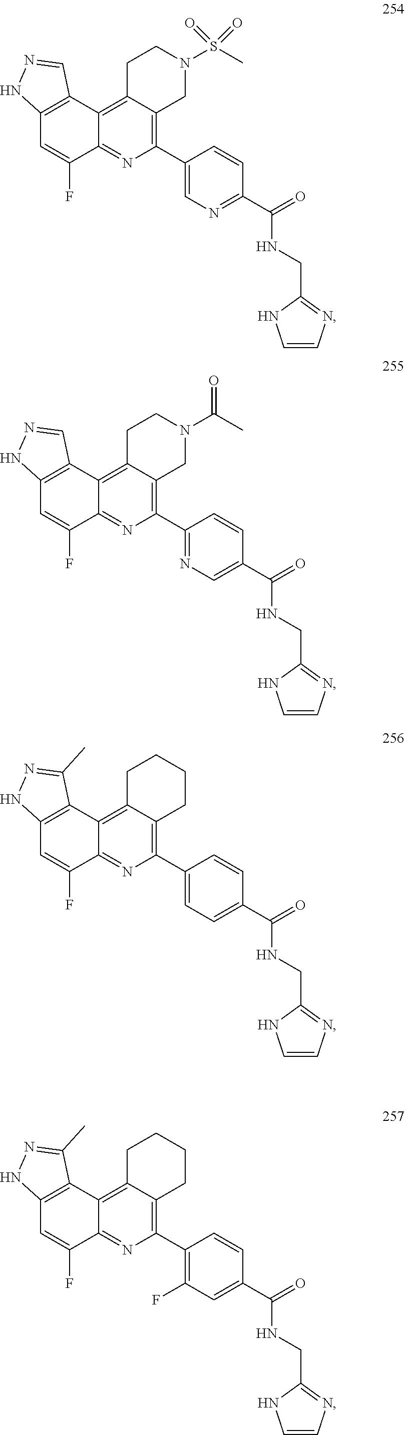 [SMILES: CS(=O)(=O)N1CCc2c(c(-c3ccc(C(=O)NCc4ncc[nH]4)nc3)nc3c(F)cc4[nH]ncc4c23)C1 ; CC(=O)N1CCc2c(c(-c3ccc(C(=O)NCc4ncc[nH]4)cn3)nc3c(F)cc4[nH]ncc4c23)C1 ; Cc1n[nH]c2cc(F)c3nc(-c4ccc(C(=O)NCc5ncc[nH]5)cc4)c4c(c3c12)CCCC4 ; Cc1n[nH]c2cc(F)c3nc(-c4ccc(C(=O)NCc5ncc[nH]5)cc4F)c4c(c3c12)CCCC4]

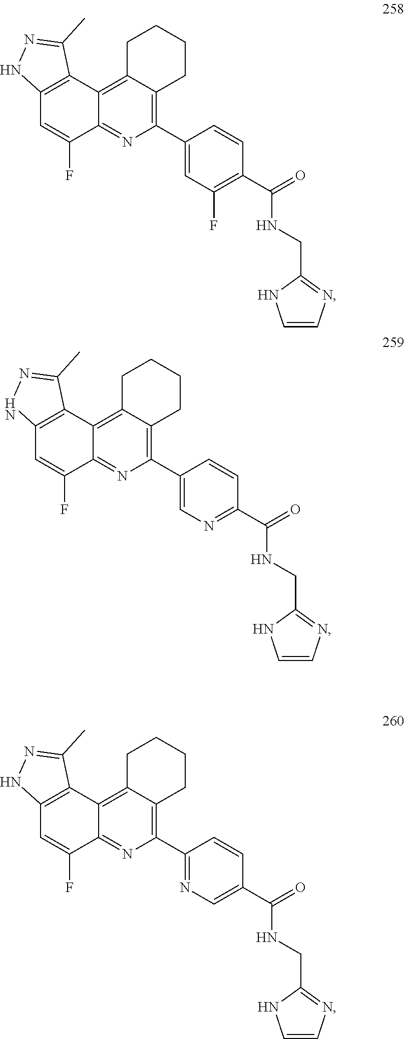 [SMILES: Cc1n[nH]c2cc(F)c3nc(-c4ccc(C(=O)NCc5ncc[nH]5)c(F)c4)c4c(c3c12)CCCC4 ; Cc1n[nH]c2cc(F)c3nc(-c4ccc(C(=O)NCc5ncc[nH]5)nc4)c4c(c3c12)CCCC4 ; Cc1n[nH]c2cc(F)c3nc(-c4ccc(C(=O)NCc5ncc[nH]5)cn4)c4c(c3c12)CCCC4]

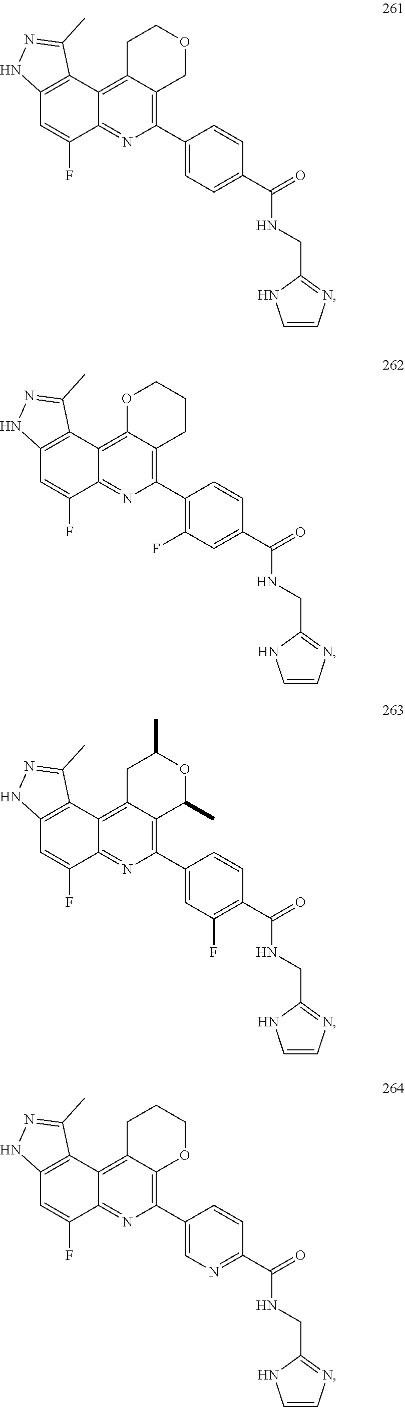 [SMILES: Cc1n[nH]c2cc(F)c3nc(-c4ccc(C(=O)NCc5ncc[nH]5)cc4)c4c(c3c12)CCOC4 ; Cc1n[nH]c2cc(F)c3nc(-c4ccc(C(=O)NCc5ncc[nH]5)cc4F)c4c(c3c12)OCCC4 ; Cc1n[nH]c2cc(F)c3nc(-c4ccc(C(=O)NCc5ncc[nH]5)c(F)c4)c4c(c3c12)C[C@@H](C)O[C@H]4C ; Cc1n[nH]c2cc(F)c3nc(-c4ccc(C(=O)NCc5ncc[nH]5)nc4)c4c(c3c12)CCCO4]

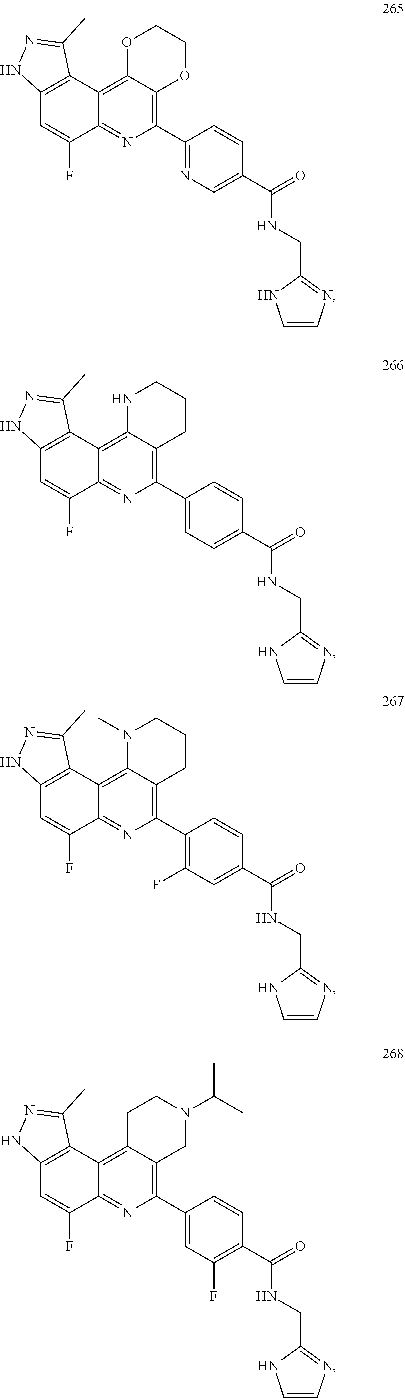 [SMILES: Cc1n[nH]c2cc(F)c3nc(-c4ccc(C(=O)NCc5ncc[nH]5)cn4)c4c(c3c12)OCCO4 ; Cc1n[nH]c2cc(F)c3nc(-c4ccc(C(=O)NCc5ncc[nH]5)cc4)c4c(c3c12)NCCC4 ; Cc1n[nH]c2cc(F)c3nc(-c4ccc(C(=O)NCc5ncc[nH]5)cc4F)c4c(c3c12)N(C)CCC4 ; Cc1n[nH]c2cc(F)c3nc(-c4ccc(C(=O)NCc5ncc[nH]5)c(F)c4)c4c(c3c12)CCN(C(C)C)C4]

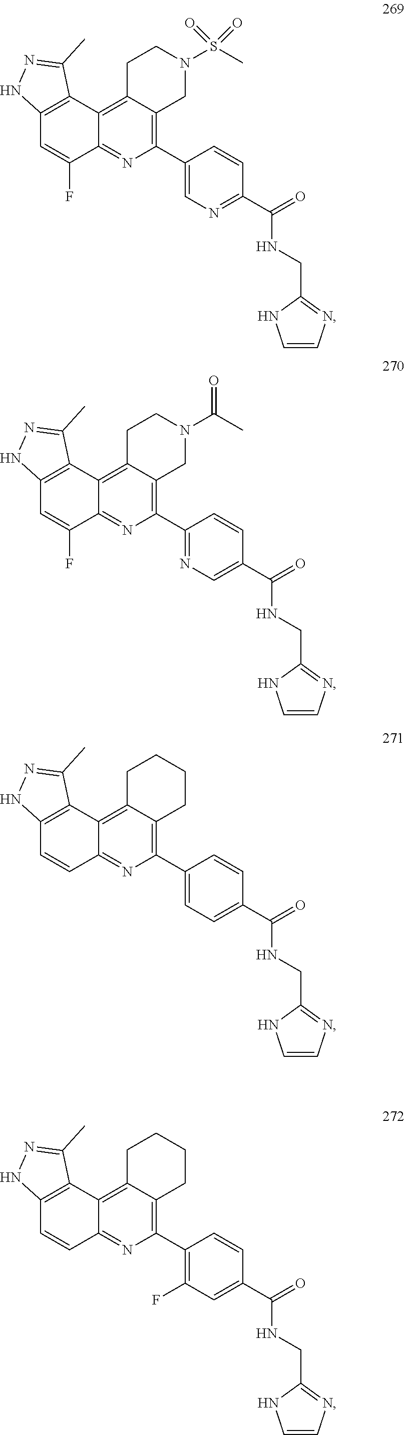 [SMILES: Cc1n[nH]c2cc(F)c3nc(-c4ccc(C(=O)NCc5ncc[nH]5)nc4)c4c(c3c12)CCN(S(C)(=O)=O)C4 ; CC(=O)N1CCc2c(c(-c3ccc(C(=O)NCc4ncc[nH]4)cn3)nc3c(F)cc4[nH]nc(C)c4c23)C1 ; Cc1n[nH]c2ccc3nc(-c4ccc(C(=O)NCc5ncc[nH]5)cc4)c4c(c3c12)CCCC4 ; Cc1n[nH]c2ccc3nc(-c4ccc(C(=O)NCc5ncc[nH]5)cc4F)c4c(c3c12)CCCC4]

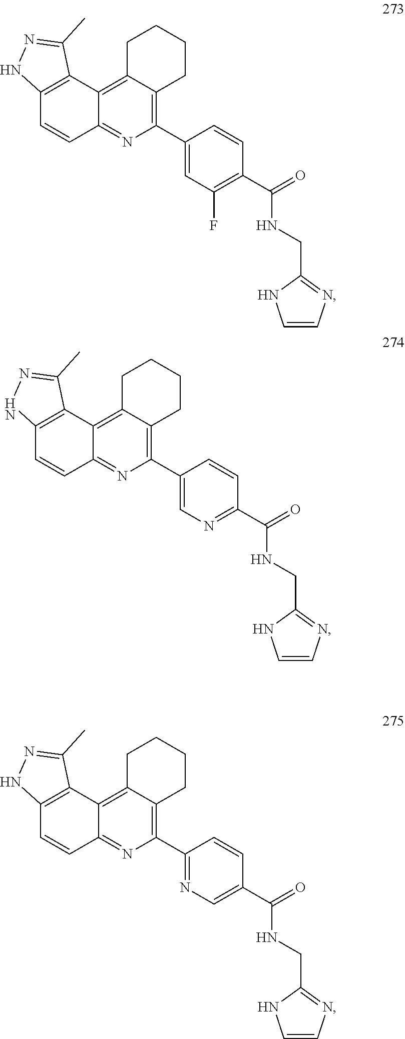 [SMILES: Cc1n[nH]c2ccc3nc(-c4ccc(C(=O)NCc5ncc[nH]5)c(F)c4)c4c(c3c12)CCCC4 ; Cc1n[nH]c2ccc3nc(-c4ccc(C(=O)NCc5ncc[nH]5)nc4)c4c(c3c12)CCCC4 ; Cc1n[nH]c2ccc3nc(-c4ccc(C(=O)NCc5ncc[nH]5)cn4)c4c(c3c12)CCCC4]

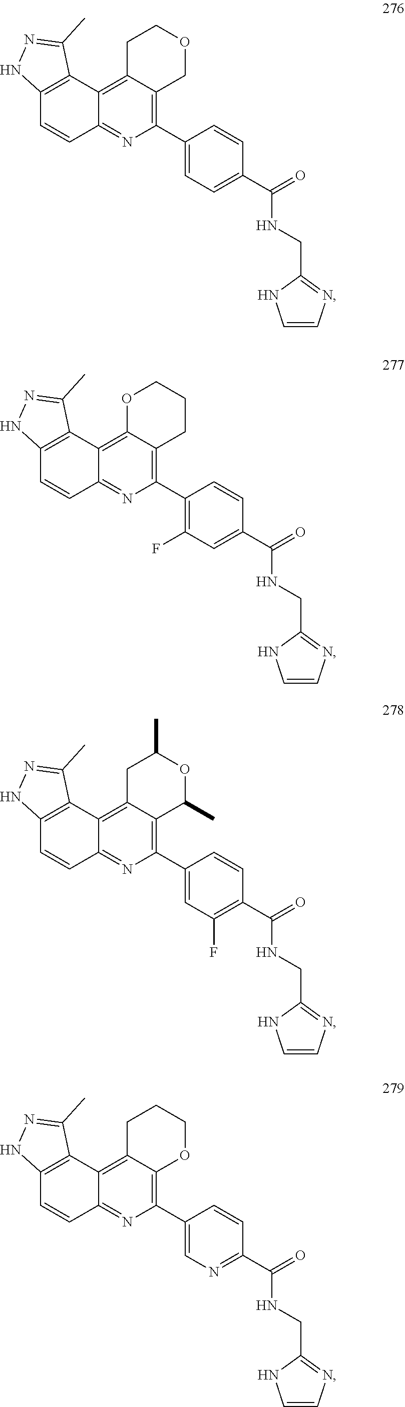 [SMILES: Cc1n[nH]c2ccc3nc(-c4ccc(C(=O)NCc5ncc[nH]5)cc4)c4c(c3c12)CCOC4 ; Cc1n[nH]c2ccc3nc(-c4ccc(C(=O)NCc5ncc[nH]5)cc4F)c4c(c3c12)OCCC4 ; Cc1n[nH]c2ccc3nc(-c4ccc(C(=O)NCc5ncc[nH]5)c(F)c4)c4c(c3c12)C[C@@H](C)O[C@H]4C ; Cc1n[nH]c2ccc3nc(-c4ccc(C(=O)NCc5ncc[nH]5)nc4)c4c(c3c12)CCCO4]

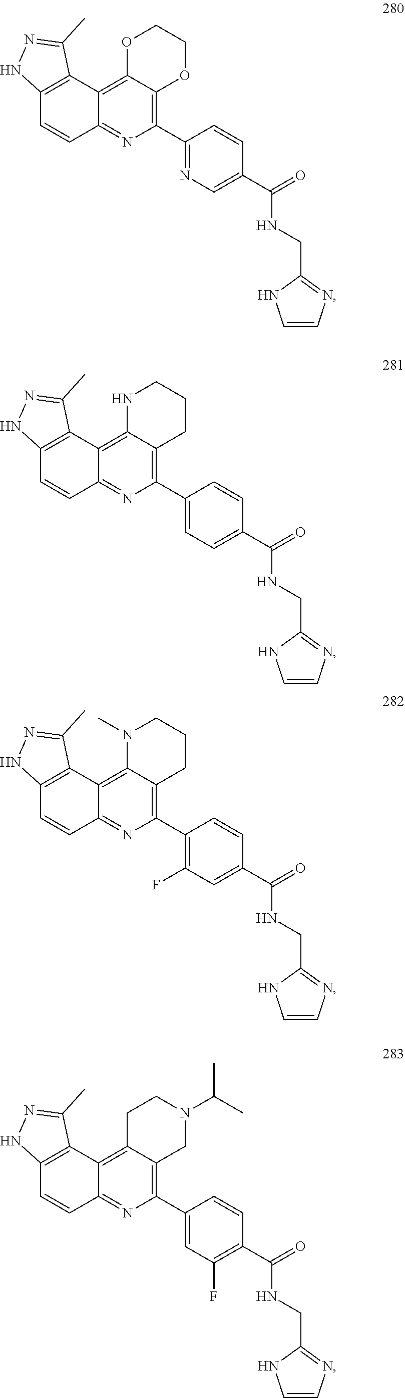 [SMILES: Cc1n[nH]c2ccc3nc(-c4ccc(C(=O)NCc5ncc[nH]5)cn4)c4c(c3c12)OCCO4 ; Cc1n[nH]c2ccc3nc(-c4ccc(C(=O)NCc5ncc[nH]5)cc4)c4c(c3c12)NCCC4 ; Cc1n[nH]c2ccc3nc(-c4ccc(C(=O)NCc5ncc[nH]5)cc4F)c4c(c3c12)N(C)CCC4 ; Cc1n[nH]c2ccc3nc(-c4ccc(C(=O)NCc5ncc[nH]5)c(F)c4)c4c(c3c12)CCN(C(C)C)C4]

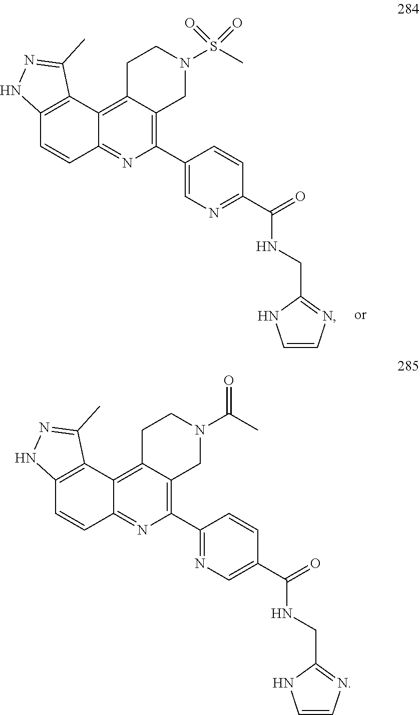 [SMILES: Cc1n[nH]c2ccc3nc(-c4ccc(C(=O)NCc5ncc[nH]5)nc4)c4c(c3c12)CCN(S(C)(=O)=O)C4 ; "or" ; CC(=O)N1CCc2c(c(-c3ccc(C(=O)NCc4ncc[nH]4)cn3)nc3ccc4[nH]nc(C)c4c23)C1]

The compound can have a structure of:

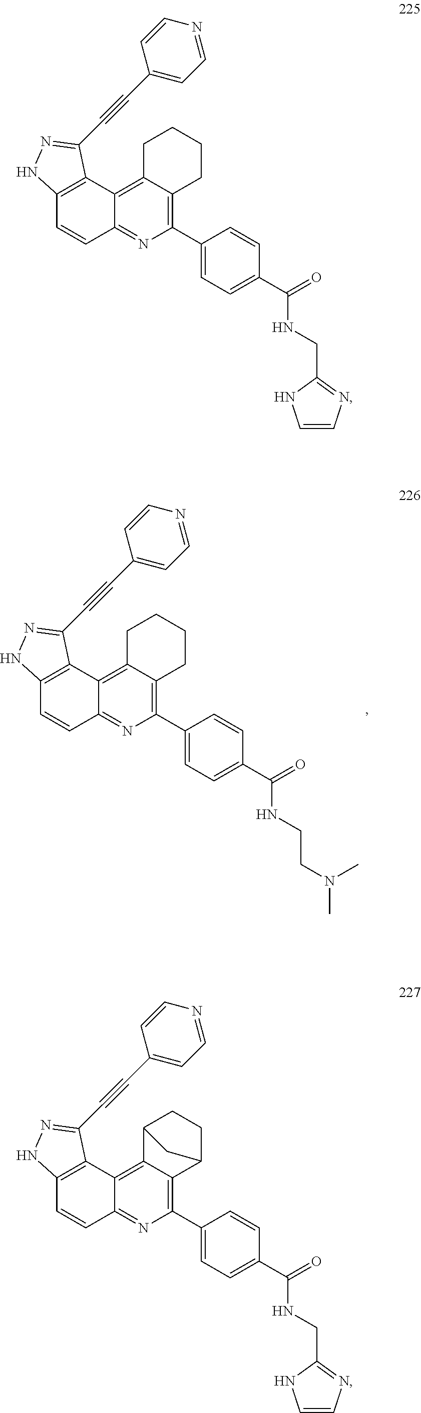 [SMILES: O=C(NCc1ncc[nH]1)c1ccc(-c2nc3ccc4[nH]nc(C#Cc5ccncc5)c4c3c3c2CCCC3)cc1 ; CN(C)CCNC(=O)c1ccc(-c2nc3ccc4[nH]nc(C#Cc5ccncc5)c4c3c3c2CCCC3)cc1 ; O=C(NCc1ncc[nH]1)c1ccc(-c2nc3ccc4[nH]nc(C#Cc5ccncc5)c4c3c3c2C2CCC3C2)cc1]

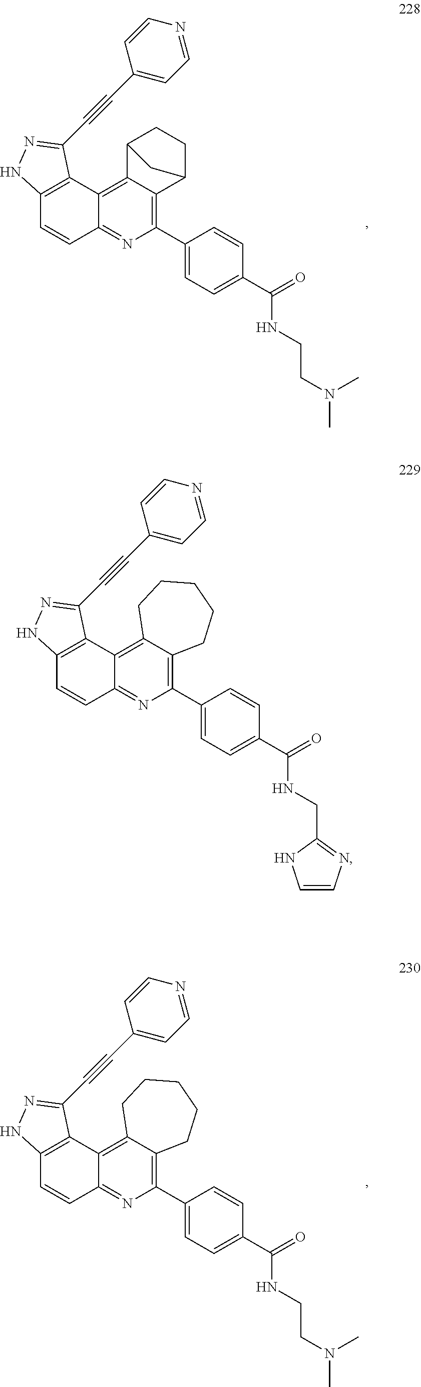 [SMILES: CN(C)CCNC(=O)c1ccc(-c2nc3ccc4[nH]nc(C#Cc5ccncc5)c4c3c3c2C2CCC3C2)cc1 ; O=C(NCc1ncc[nH]1)c1ccc(-c2nc3ccc4[nH]nc(C#Cc5ccncc5)c4c3c3c2CCCCC3)cc1 ; CN(C)CCNC(=O)c1ccc(-c2nc3ccc4[nH]nc(C#Cc5ccncc5)c4c3c3c2CCCCC3)cc1]

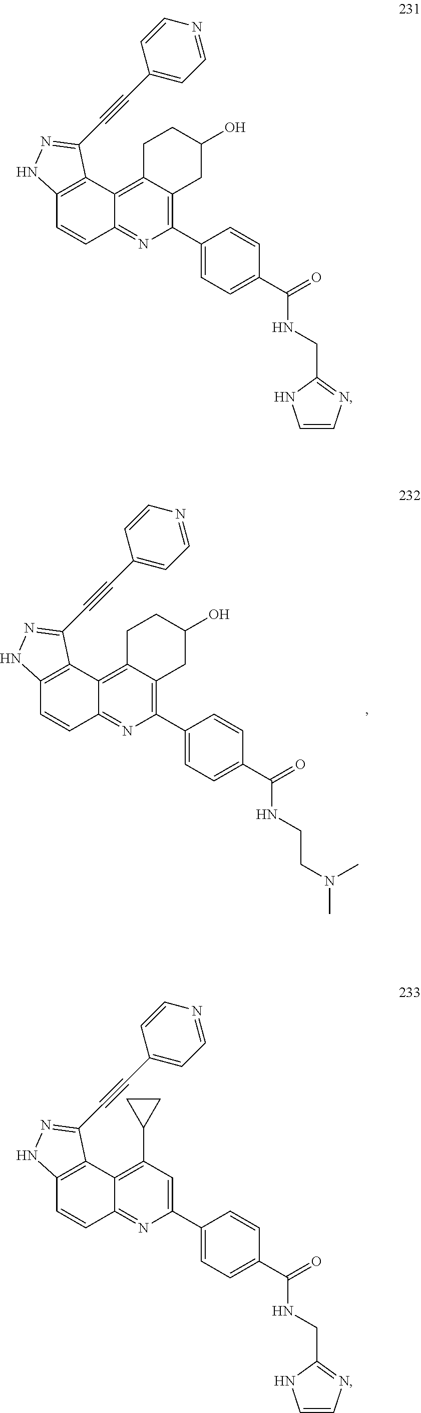 [SMILES: O=C(NCc1ncc[nH]1)c1ccc(-c2nc3ccc4[nH]nc(C#Cc5ccncc5)c4c3c3c2CC(O)CC3)cc1 ; CN(C)CCNC(=O)c1ccc(-c2nc3ccc4[nH]nc(C#Cc5ccncc5)c4c3c3c2CC(O)CC3)cc1 ; O=C(NCc1ncc[nH]1)c1ccc(-c2cc(C3CC3)c3c(ccc4[nH]nc(C#Cc5ccncc5)c43)n2)cc1]

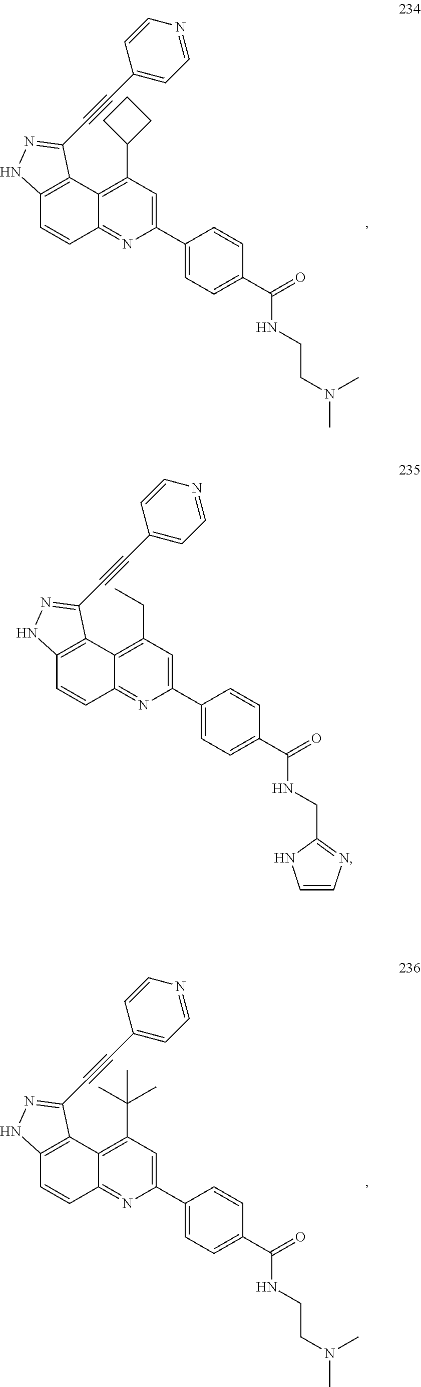 [SMILES: CN(C)CCNC(=O)c1ccc(-c2cc(C3CCC3)c3c(ccc4[nH]nc(C#Cc5ccncc5)c43)n2)cc1 ; CCc1cc(-c2ccc(C(=O)NCc3ncc[nH]3)cc2)nc2ccc3[nH]nc(C#Cc4ccncc4)c3c12 ; CN(C)CCNC(=O)c1ccc(-c2cc(C(C)(C)C)c3c(ccc4[nH]nc(C#Cc5ccncc5)c43)n2)cc1]

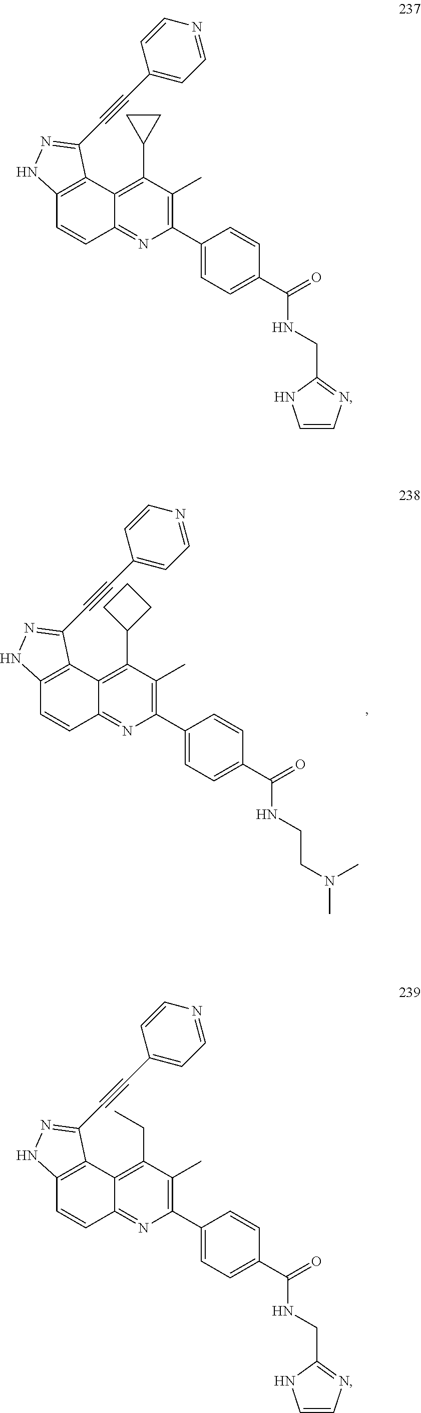 [SMILES: Cc1c(-c2ccc(C(=O)NCc3ncc[nH]3)cc2)nc2ccc3[nH]nc(C#Cc4ccncc4)c3c2c1C1CC1 ; Cc1c(-c2ccc(C(=O)NCCN(C)C)cc2)nc2ccc3[nH]nc(C#Cc4ccncc4)c3c2c1C1CCC1 ; CCc1c(C)c(-c2ccc(C(=O)NCc3ncc[nH]3)cc2)nc2ccc3[nH]nc(C#Cc4ccncc4)c3c12]

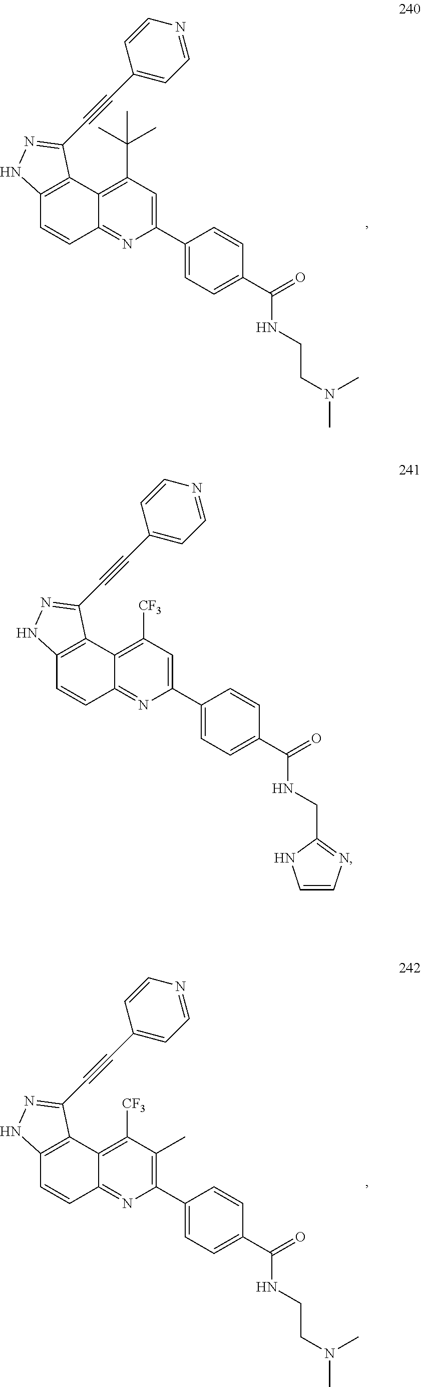 [SMILES: CN(C)CCNC(=O)c1ccc(-c2cc(C(C)(C)C)c3c(ccc4[nH]nc(C#Cc5ccncc5)c43)n2)cc1 ; O=C(NCc1ncc[nH]1)c1ccc(-c2cc(C(F)(F)F)c3c(ccc4[nH]nc(C#Cc5ccncc5)c43)n2)cc1 ; Cc1c(-c2ccc(C(=O)NCCN(C)C)cc2)nc2ccc3[nH]nc(C#Cc4ccncc4)c3c2c1C(F)(F)F]

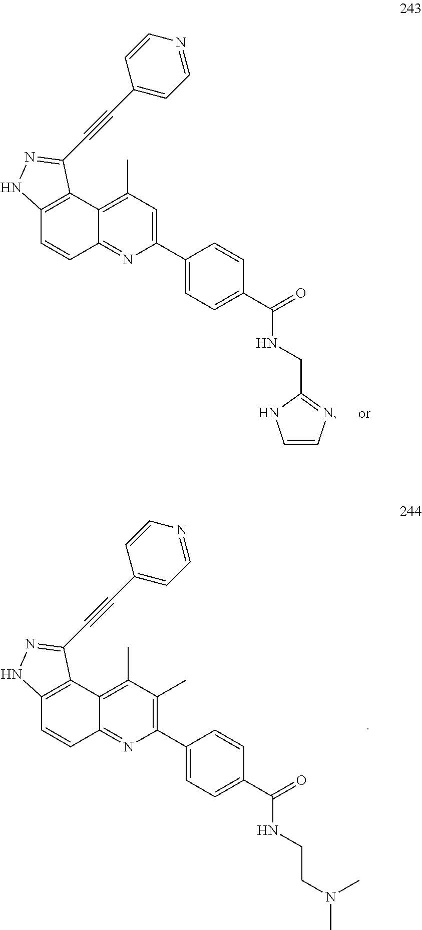 [SMILES: Cc1cc(-c2ccc(C(=O)NCc3ncc[nH]3)cc2)nc2ccc3[nH]nc(C#Cc4ccncc4)c3c12 ; Cc1c(-c2ccc(C(=O)NCCN(C)C)cc2)nc2ccc3[nH]nc(C#Cc4ccncc4)c3c2c1C]

The compound can have a structure of:

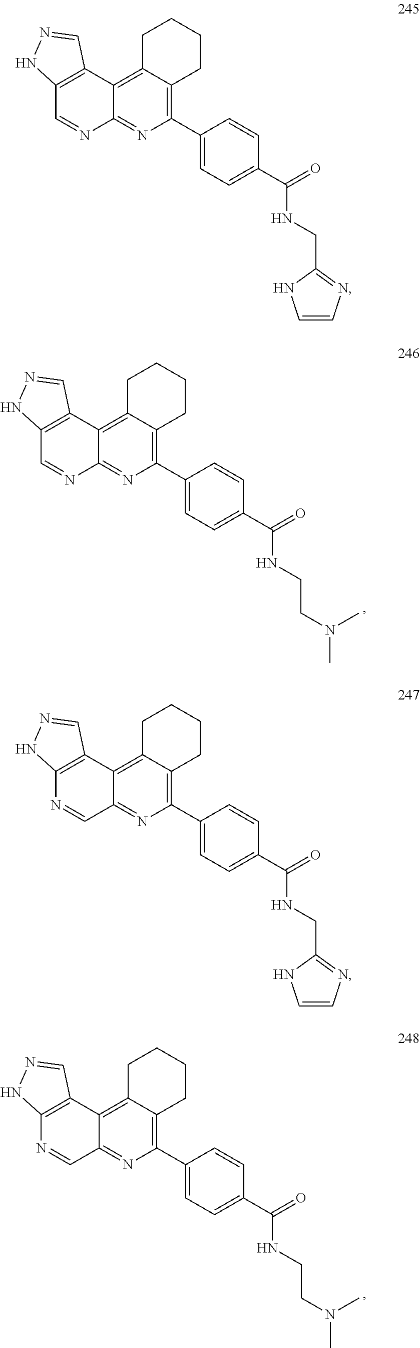 [SMILES: O=C(NCc1ncc[nH]1)c1ccc(-c2nc3ncc4[nH]ncc4c3c3c2CCCC3)cc1 ; CN(C)CCNC(=O)c1ccc(-c2nc3ncc4[nH]ncc4c3c3c2CCCC3)cc1 ; O=C(NCc1ncc[nH]1)c1ccc(-c2nc3cnc4[nH]ncc4c3c3c2CCCC3)cc1 ; CN(C)CCNC(=O)c1ccc(-c2nc3cnc4[nH]ncc4c3c3c2CCCC3)cc1]

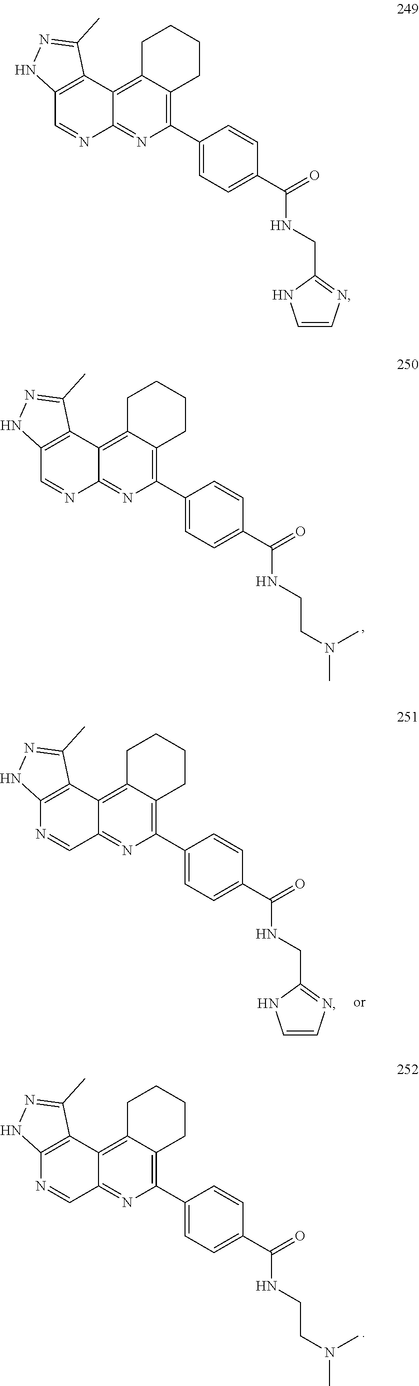 [SMILES: Cc1n[nH]c2cnc3nc(-c4ccc(C(=O)NCc5ncc[nH]5)cc4)c4c(c3c12)CCCC4 ; Cc1n[nH]c2cnc3nc(-c4ccc(C(=O)NCCN(C)C)cc4)c4c(c3c12)CCCC4 ; Cc1n[nH]c2ncc3nc(-c4ccc(C(=O)NCc5ncc[nH]5)cc4)c4c(c3c12)CCCC4 ; "or" ; Cc1n[nH]c2ncc3nc(-c4ccc(C(=O)NCCN(C)C)cc4)c4c(c3c12)CCCC4]

The compound can have a structure of:

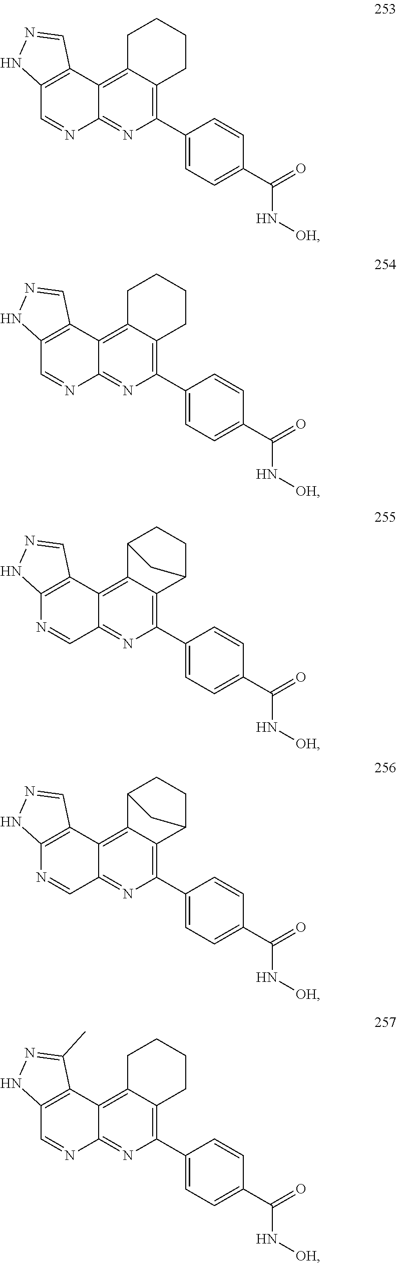 [SMILES: O=C(NO)c1ccc(-c2nc3ncc4[nH]ncc4c3c3c2CCCC3)cc1 ; O=C(NO)c1ccc(-c2nc3ncc4[nH]ncc4c3c3c2CCCC3)cc1 ; O=C(NO)c1ccc(-c2nc3cnc4[nH]ncc4c3c3c2C2CCC3C2)cc1 ; O=C(NO)c1ccc(-c2nc3cnc4[nH]ncc4c3c3c2C2CCC3C2)cc1 ; Cc1n[nH]c2cnc3nc(-c4ccc(C(=O)NO)cc4)c4c(c3c12)CCCC4]

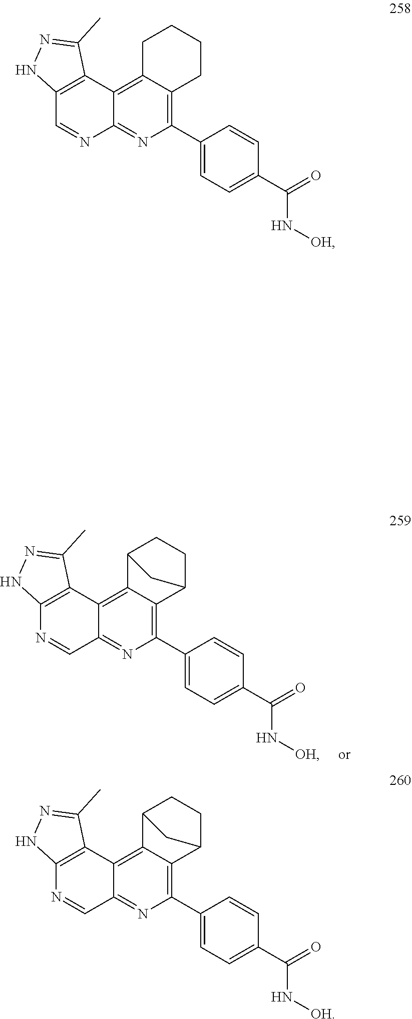 [SMILES: Cc1n[nH]c2cnc3nc(-c4ccc(C(=O)NO)cc4)c4c(c3c12)CCCC4 ; Cc1n[nH]c2ncc3nc(-c4ccc(C(=O)NO)cc4)c4c(c3c12)C1CCC4C1 ; Cc1n[nH]c2ncc3nc(-c4ccc(C(=O)NO)cc4)c4c(c3c12)C1CCC4C1]

The compound can have a structure of:

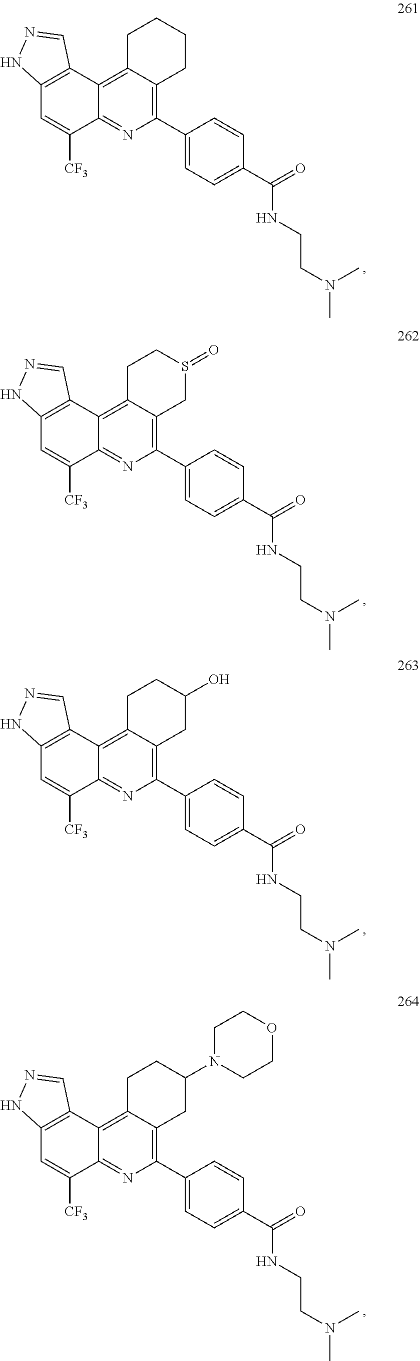 [SMILES: CN(C)CCNC(=O)c1ccc(-c2nc3c(C(F)(F)F)cc4[nH]ncc4c3c3c2CCCC3)cc1 ; CN(C)CCNC(=O)c1ccc(-c2nc3c(C(F)(F)F)cc4[nH]ncc4c3c3c2CS(=O)CC3)cc1 ; CN(C)CCNC(=O)c1ccc(-c2nc3c(C(F)(F)F)cc4[nH]ncc4c3c3c2CC(O)CC3)cc1 ; CN(C)CCNC(=O)c1ccc(-c2nc3c(C(F)(F)F)cc4[nH]ncc4c3c3c2CC(N2CCOCC2)CC3)cc1]

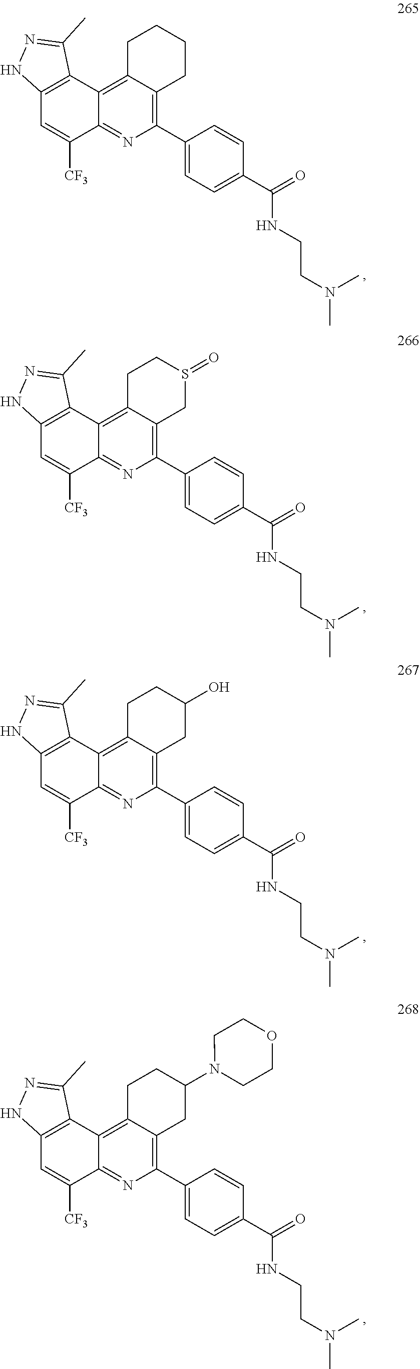 [SMILES: Cc1n[nH]c2cc(C(F)(F)F)c3nc(-c4ccc(C(=O)NCCN(C)C)cc4)c4c(c3c12)CCCC4 ; Cc1n[nH]c2cc(C(F)(F)F)c3nc(-c4ccc(C(=O)NCCN(C)C)cc4)c4c(c3c12)CCS(=O)C4 ; Cc1n[nH]c2cc(C(F)(F)F)c3nc(-c4ccc(C(=O)NCCN(C)C)cc4)c4c(c3c12)CCC(O)C4 ; Cc1n[nH]c2cc(C(F)(F)F)c3nc(-c4ccc(C(=O)NCCN(C)C)cc4)c4c(c3c12)CCC(N1CCOCC1)C4]

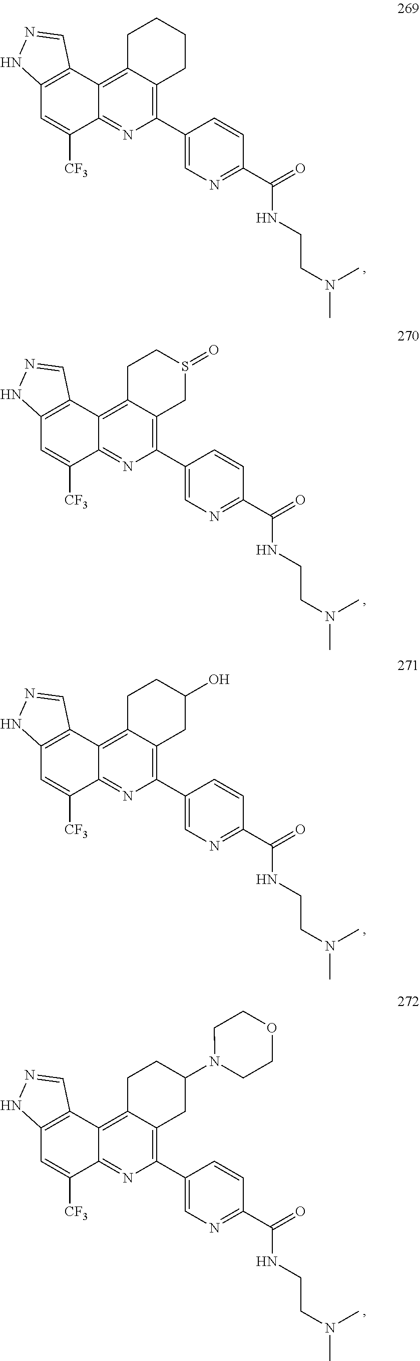 [SMILES: CN(C)CCNC(=O)c1ccc(-c2nc3c(C(F)(F)F)cc4[nH]ncc4c3c3c2CCCC3)cn1 ; CN(C)CCNC(=O)c1ccc(-c2nc3c(C(F)(F)F)cc4[nH]ncc4c3c3c2CS(=O)CC3)cn1 ; CN(C)CCNC(=O)c1ccc(-c2nc3c(C(F)(F)F)cc4[nH]ncc4c3c3c2CC(O)CC3)cn1 ; CN(C)CCNC(=O)c1ccc(-c2nc3c(C(F)(F)F)cc4[nH]ncc4c3c3c2CC(N2CCOCC2)CC3)cn1]

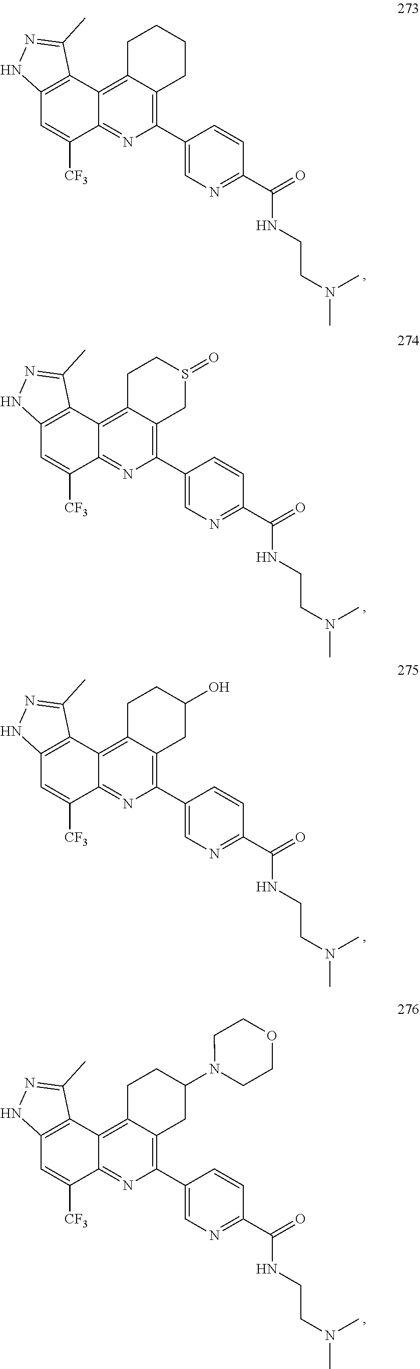 [SMILES: Cc1n[nH]c2cc(C(F)(F)F)c3nc(-c4ccc(C(=O)NCCN(C)C)nc4)c4c(c3c12)CCCC4 ; Cc1n[nH]c2cc(C(F)(F)F)c3nc(-c4ccc(C(=O)NCCN(C)C)nc4)c4c(c3c12)CCS(=O)C4 ; Cc1n[nH]c2cc(C(F)(F)F)c3nc(-c4ccc(C(=O)NCCN(C)C)nc4)c4c(c3c12)CCC(O)C4 ; Cc1n[nH]c2cc(C(F)(F)F)c3nc(-c4ccc(C(=O)NCCN(C)C)nc4)c4c(c3c12)CCC(N1CCOCC1)C4]

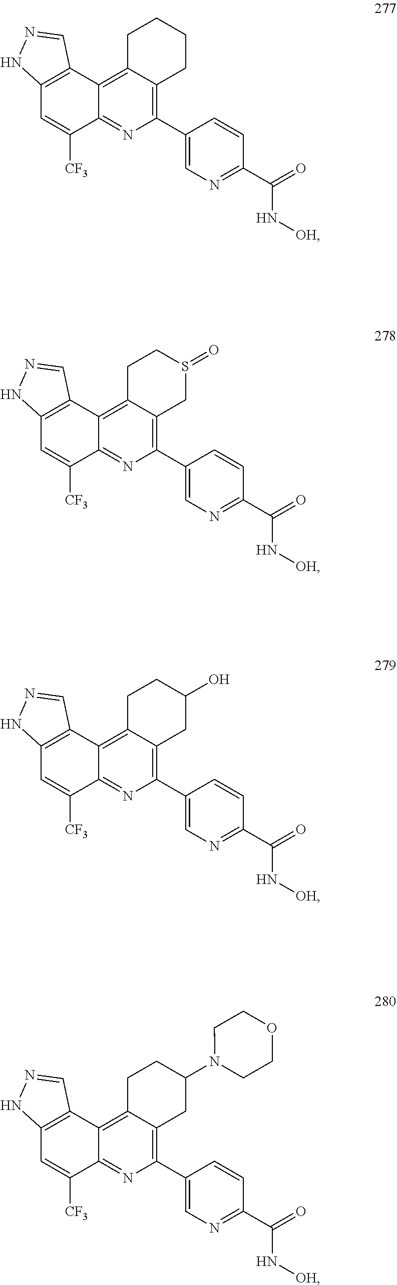 [SMILES: O=C(NO)c1ccc(-c2nc3c(C(F)(F)F)cc4[nH]ncc4c3c3c2CCCC3)cn1 ; O=C(NO)c1ccc(-c2nc3c(C(F)(F)F)cc4[nH]ncc4c3c3c2CS(=O)CC3)cn1 ; O=C(NO)c1ccc(-c2nc3c(C(F)(F)F)cc4[nH]ncc4c3c3c2CC(O)CC3)cn1 ; O=C(NO)c1ccc(-c2nc3c(C(F)(F)F)cc4[nH]ncc4c3c3c2CC(N2CCOCC2)CC3)cn1]

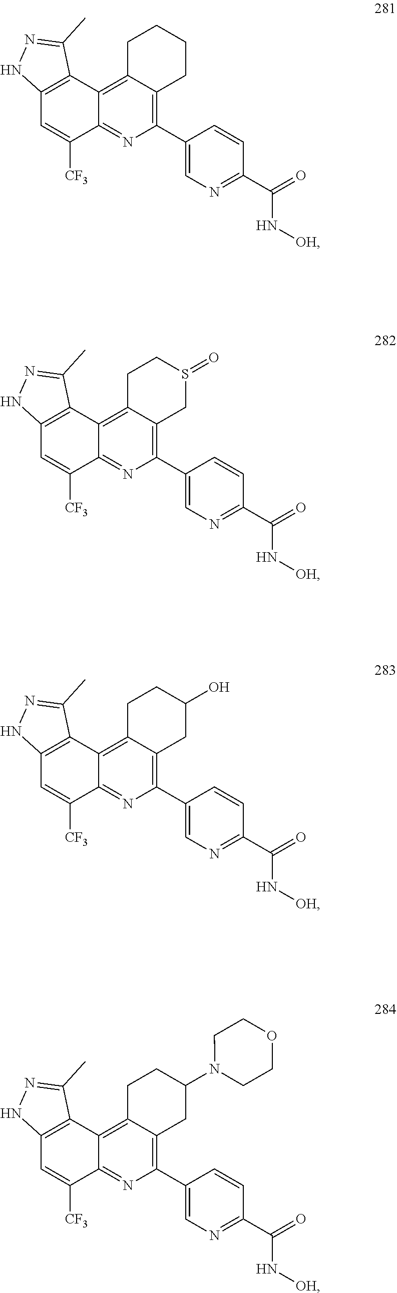 [SMILES: Cc1n[nH]c2cc(C(F)(F)F)c3nc(-c4ccc(C(=O)NO)nc4)c4c(c3c12)CCCC4 ; Cc1n[nH]c2cc(C(F)(F)F)c3nc(-c4ccc(C(=O)NO)nc4)c4c(c3c12)CCS(=O)C4 ; Cc1n[nH]c2cc(C(F)(F)F)c3nc(-c4ccc(C(=O)NO)nc4)c4c(c3c12)CCC(O)C4 ; Cc1n[nH]c2cc(C(F)(F)F)c3nc(-c4ccc(C(=O)NO)nc4)c4c(c3c12)CCC(N1CCOCC1)C4]

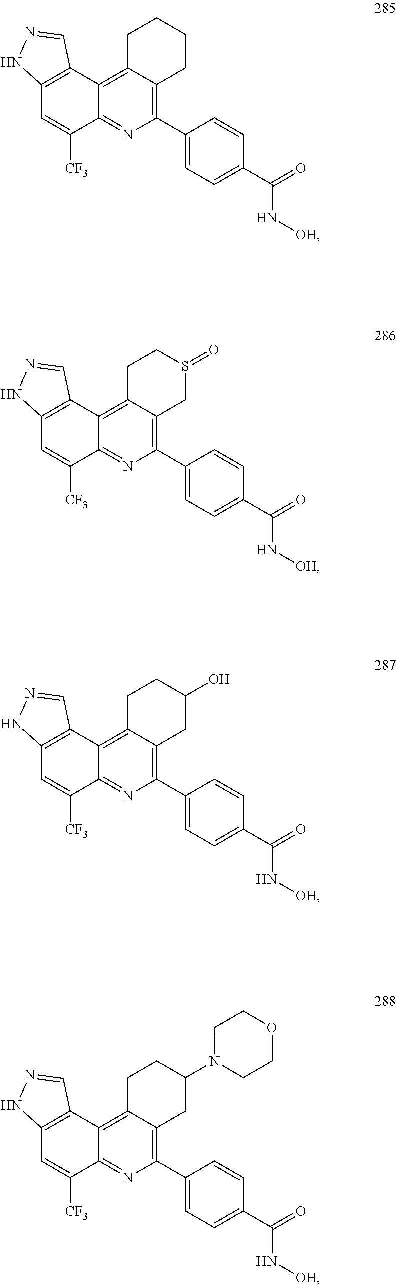 [SMILES: O=C(NO)c1ccc(-c2nc3c(C(F)(F)F)cc4[nH]ncc4c3c3c2CCCC3)cc1 ; O=C(NO)c1ccc(-c2nc3c(C(F)(F)F)cc4[nH]ncc4c3c3c2CS(=O)CC3)cc1 ; O=C(NO)c1ccc(-c2nc3c(C(F)(F)F)cc4[nH]ncc4c3c3c2CC(O)CC3)cc1 ; O=C(NO)c1ccc(-c2nc3c(C(F)(F)F)cc4[nH]ncc4c3c3c2CC(N2CCOCC2)CC3)cc1]

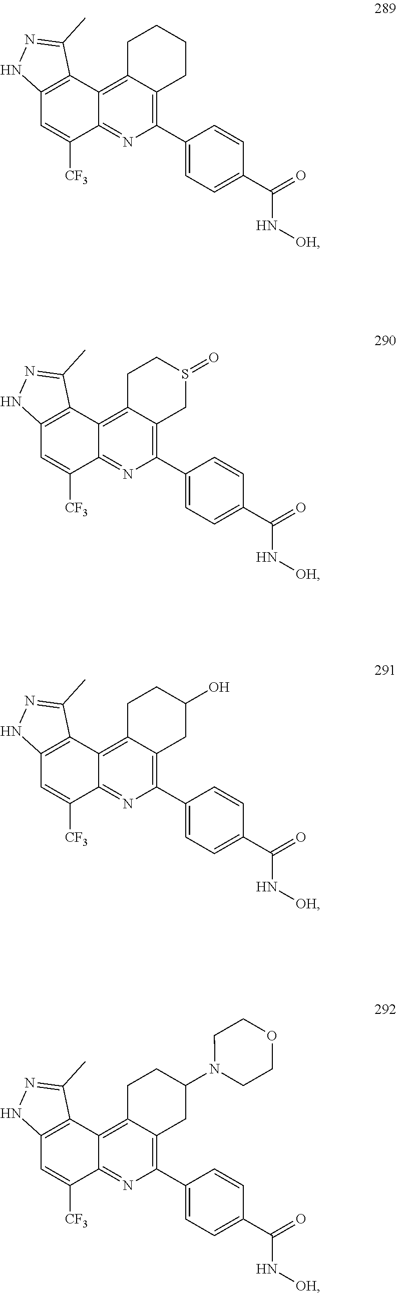 [SMILES: Cc1n[nH]c2cc(C(F)(F)F)c3nc(-c4ccc(C(=O)NO)cc4)c4c(c3c12)CCCC4 ; Cc1n[nH]c2cc(C(F)(F)F)c3nc(-c4ccc(C(=O)NO)cc4)c4c(c3c12)CCS(=O)C4 ; Cc1n[nH]c2cc(C(F)(F)F)c3nc(-c4ccc(C(=O)NO)cc4)c4c(c3c12)CCC(O)C4 ; Cc1n[nH]c2cc(C(F)(F)F)c3nc(-c4ccc(C(=O)NO)cc4)c4c(c3c12)CCC(N1CCOCC1)C4]

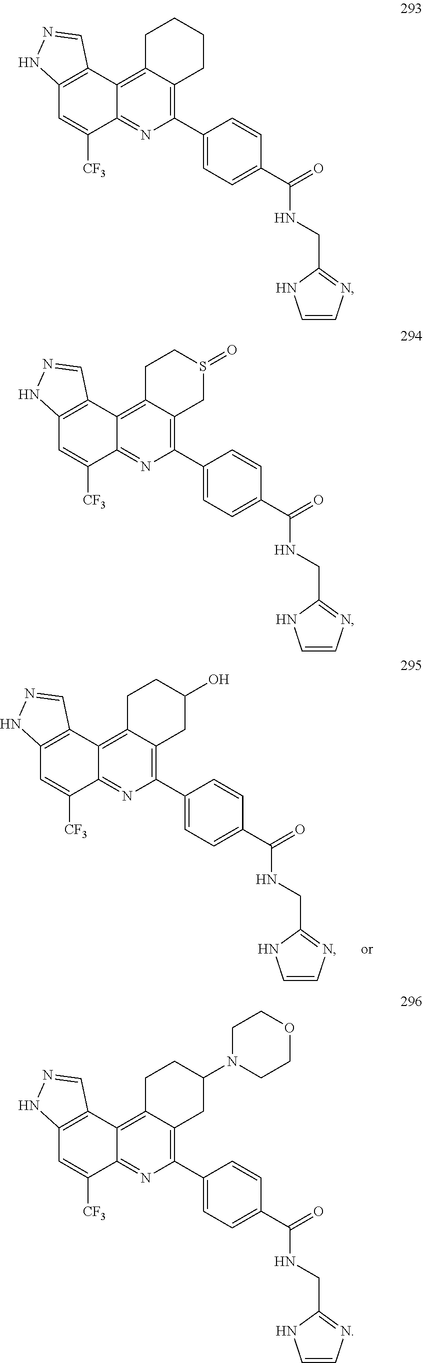

A PROTAC conjugate is also provided. In certain embodiments, the PROTAC conjugate has a chemical structure of formula X:

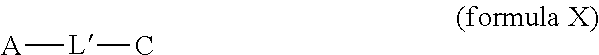

or is a pharmaceutically acceptable salt thereof, wherein:
A is a radical of any of the compounds hereof;
L′ is a linker that binds A and D or is absent; and
D is a ubiquitin pathway protein binding moiety.

Uses of a compound hereof or a conjugate hereof, or a pharmaceutically acceptable salt of a compound or conjugate hereof in the manufacture of a medicament for the treatment of a disease in a subject is also provided. In certain embodiments, the disease comprises a disease where modulation of a kinase reduces the severity of such disease in the subject. The disease can be cancer, diabetes, an inflammatory disease, or a neurological disease, for example.

Pharmaceutical compositions are also provided. A pharmaceutical composition hereof can comprise any of the compounds hereof, a conjugate hereof, or a pharmaceutically acceptable salt, N-oxide, hydrate, solvent, tautomer, or optical isomer of the compound or conjugate; and a pharmaceutically acceptable carrier and/or diluent. The pharmaceutical composition can further comprise a pharmaceutically acceptable excipient.

Methods of treating a disease state (e.g., a cancer) in a subject are also provided. In certain embodiments, the method comprises administering to the subject a therapeutically effective amount of: any of the compounds hereof, a conjugate hereof, or a pharmaceutically acceptable salt, N-oxide, hydrate, solvate, tautomer, or optical isomer of the compound or conjugate; or a pharmaceutical composition comprising one or more of a compound hereof, a conjugate hereof, or a pharmaceutically acceptable salt, N-oxide, hydrate, solvate, tautomer, or optical isomer of the compound or conjugate.

In certain embodiments of the method, the compound or conjugate comprises a compound having a structure of:

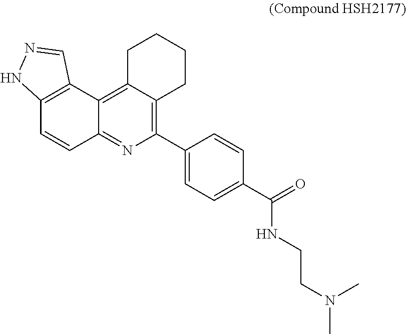 [SMILES: CN(C)CCNC(=O)c1ccc(-c2nc3ccc4[nH]ncc4c3c3c2CCCC3)cc1]

or a pharmaceutically acceptable salt thereof.

In certain embodiments of the method, the compound or conjugate comprises a compound having a structure of:

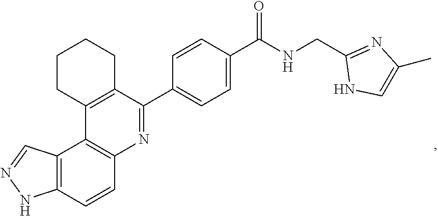 [SMILES: Cc1c[nH]c(CNC(=O)c2ccc(-c3nc4ccc5[nH]ncc5c4c4c3CCCC4)cc2)n1]

or a pharmaceutically acceptable salt thereof.

The method can further comprise administering to the subject a second therapy. The second therapy can comprise an effective amount of a chemotherapeutic agent, an immunotherapeutic agent, or a hormone therapeutic agent; or radiation therapy.

The cancer can be acute myeloid leukemia (AML). In certain embodiments, the cancer is selected from the group consisting of AML, chronic myeloid leukemia, ovarian cancer, cervical cancer, pancreatic cancer, breast cancer, brain cancer, skin cancer, lung cancer, prostate cancer, lymphoma, leukemia, colon cancer, gastric carcinoma, head cancer, neck cancer, thyroid cancer, kidney cancer, liver cancer, and stomach cancer.

In certain embodiments of the method, administering the effective amount to the subject inhibits a CDK in the subject at an IC₅₀ of about 50 nM or lower (such as 50 nM or lower). In certain embodiments, administering the effective amount to the subject inhibits a CDK in the subject at an IC₅₀ of about 30 nM or lower (such as 30 nM or lower).

Administering the effective amount to the subject can inhibit both FLT3 and haspin kinase with specificity in the subject, for example where a dual inhibitor compound is administered.

Compounds and/or conjugates described herein are further provided for use in the treatment of a disease state modulated by one or more kinase. For example, the disease state can be cancer and the one or more kinases can be CDK, FLT3, and/or haspin kinase.

### BRIEF DESCRIPTION OF DRAWINGS

The disclosed embodiments and other features, advantages, and aspects contained herein, and the matter of attaining them, will become apparent in light of the following detailed description of various exemplary embodiments of the present disclosure. Such detailed description will be better understood when taken in conjunction with the accompanying drawings.

FIG. 1 shows a schematic representation of the modification of compound HSD1217 to produce Compound HSH2177 and HSD1993, which are both potent inhibitors of CDK2.

FIG. 2A shows fusion of indazole with quinoline to afford a novel hinge binder, 3H-pyrazolo[4,3-f]quinoline scaffold core.

FIG. 2B shows examples of compounds having C-7 substitution of the 3H-pyrazolo[4,3-f]quinoline moiety, which affects kinase targeting.

FIG. 3 shows graphical data related to percent viability of the SNU-16 cell line (a human gastric carcinoma cell line) when treated with 1 μM of each of the identified compounds.

FIGS. 4A-4D show data related to the percent proliferation of SNU-16 cell line versus Compound HSH2177 (FIG. 4A), Compound HSH3014 (FIG. 4B), Compound HSH3180 (FIG. 4C), and Compound HSH3194 (FIG. 4D).

FIG. 5 shows graphical data related to percent viability of the KCL22-IR cell line (a chronic myeloid leukemia cell line) when treated with 1 μM of each of the identified compounds.

FIGS. 6A-6D show data related to the percent proliferation of KCL22-IR cell line versus Compound HSH2177 (FIG. 6A), Compound HSH3014 (FIG. 6B), Compound HSH3194 (FIG. 6C), and Compound HSH3180 (FIG. 6D).

FIG. 7 shows graphical data related to percent viability of the Molm-14 (D835Y) cell line (a human AML cell line with a mutation at D835Y) when treated with 1 μM of each of the identified compounds.

FIGS. 8A-8D show data related to the percent proliferation of Molm-14 (D835Y) cell line versus Compound HSH2177 (FIG. 8A), Compound HSH3014 (FIG. 8B), Compound HSH3194 (FIG. 8C), and Compound HSH3180 (FIG. 8D).

FIG. 9 shows graphical data related to percent viability of the Molm-14 (F69IL) cell line (a human AML cell line with a mutation at F691L) when treated with 1 μM of each of the identified compounds.

FIGS. 10A-10C show data related to the percent proliferation of Molm-14 (F69IL) cell line versus Compound HSH2177 (FIG. 10A), Compound HSH3194 (FIG. 10B), and Compound HSH3180 (FIG. 10C).

FIG. 11A shows a schematic of Compound HSD1217 and ATP-binding residues.

FIGS. 11B-11D show docked structures showing interactions between haspin (PDB: 2WB8) and three initial compounds: Compound 4 (FIG. 11B), Compound 5 (FIG. 2C), and Compound 6 (FIG. 11D), where the hydrogen bonds are represented identified as HYD, pi-cation interactions are identified by X, and pi-pi stacking is identified by Y. Solvent exposure is represented with grey rings surrounding atoms. Docking was performed using Glide (Schrödinger) and visualization through PyMol and Maestro.

FIGS. 12A-12C show enzyme binding and inhibition data for Compound HSK205, with FIG. 12A showing K_{d} data for haspin kinase obtained from Eurofins (each data point representing the mean and the error bars representing the SEM of triplicates), FIG. 12B showing data for Compound HSK205 tested against FLT3-ITD enzyme in 10-dose IC₅₀ duplicate mode with a 3-fold serial dilution starting at 10 μM, and FIG. 12C showing data for Compound HSK205 tested against FLT3 (D835Y) enzyme in 10-dose IC₅₀ duplicate mode with a 3-fold serial dilution starting at 10 μM (all data fitted to a non-linear regression equation using GraphPad Prism 9.0 software).

FIGS. 13A and 13B show the results of Western Blot analysis, with FIG. 13A showing Molm-14 cells treated with Compound HSK205 (5, 20 or 100 nM) or control for 24 hours (gilteritinib was used at 100 nM was a positive control), and FIG. 13B showing data from the quantification of the bands in FIG. 13A relative to an actin loading control (values are reported as means of triplicates and the error bars represents standard deviation (NS: p>0.05, *: p<0.05, p<0.01, *** p<0.001, **** p<0.0001).

FIGS. 14A and 14B show the results of Western Blot analysis, with FIG. 14A showing Molm-14 cells treated with Compound HSK205 (5, 20 or 100 nM) or control for 24 hours (gilteritinib was used at 5 nM was a positive control), and FIG. 14B showing data from the quantification of the bands in FIG. 14A relative to an actin loading control (values are reported as means of triplicates and the error bars represents standard deviation (NS: p>0.05, *: p<0.05, ** p<0.01, *** p<0.001, **** p<0.0001).

FIG. 15 is a graph of dose response curves for FLT3-driven AML cells treated with varying concentrations of Compound HSK205, Compound HSK2150, or gilteritinib (a known FLT3 inhibitor) for 72 hours, with data fitted to a non-linear regression equation using GraphPad Prism 9.0 software (each data point represents the mean and error bars represent the SEM of triplicates).

FIG. 16 is a graph of data from a DNA intercalation assay. Mitoxantrone was used as a positive control and DMSO was used as a negative control. Compound HSK205, DMSO, and mitoxantrone were tested over a range of doses from 1 mM to 10 nM. Data was fitted to a non-linear regression equation using GraphPad Prism 9.0 software (each data point represents the mean of triplicates).

While the present disclosure is susceptible to various modifications and alternative forms, exemplary embodiments thereof are shown by way of example in the drawings and are herein described in detail.

### DETAILED DESCRIPTION

While the concepts of the present disclosure are illustrated and described in detail in the description herein, results in the description are to be considered as exemplary and not restrictive in character; it being understood that only the illustrative embodiments are shown and described and that all changes and modifications that come within the spirit of the disclosure are desired to be protected.

U.S. Pat. No. 11,040,973 to Sintim et al. establishes that HSD1217, a 4-(3H-pyrazolo[4,3-f]quinolin-7-yl)benzamide containing compound synthesized via a Doebner-Povarov multi-component reaction, is a potent inhibitors of kinases, such as FLT3 kinase.

HSD1217 has the following structure:

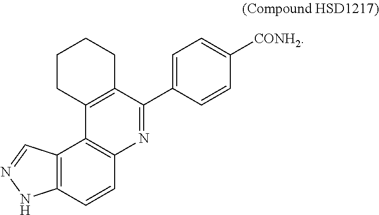 [SMILES: NC(=O)c1ccc(-c2nc3ccc4[nH]ncc4c3c3c2CCCC3)cc1]

Compound HSD1217, however, is only a moderate inhibitor of CDKs, such as CDK2 and CDK12, which are important kinase targets that have come to the forefront as potential anti-cancer targets. For example, the IC₅₀ for CDK2 inhibition by HSD1217 is 185 nM.

In certain embodiments, present disclosure provides compounds that comprise either a 2-(dimethylamino)ethyl moiety or hydroxamic acid in place of a benzamide moiety and allow for the unexpected and dramatically improved kinase targeting, as compared to at least compound HSD1217 and other conventional CDK inhibitors.

In certain embodiments, the compounds hereof are analogs of Compounds HSH2177 and/or Compounds HSD1993. Such compounds have the ability to inhibit CDK function and can be used to treat diseased states where targeting CDKs will lead to restoration of normal cellular function, such as cancer, inflammatory diseases, and certain neurological diseases.

Further, it has recently been reported that FMS-like tyrosine kinase-3 (FLT3) inhibitors have activities against other cancer related kinases, such as dual FLT3/MERTK, FLT3/MNK, FLT3/CDK, FLT3/TOPK, FLT3/PIM, or FLT3/AURKA inhibitors, where the concurrent inhibition of the FLT3 and other kinase pathways may confer some advantages in overcoming resistance. Minson et al., MRX2843, a novel dual MerTK-FLT3 inhibitor with activity against resistance-conferring FLT3 mutations in acute myeloid leukemia, Blood 124:3757 (2014); Yen et al., Identification of dual FLT3 and MNK2 inhibitor for acute myeloid leukemia treatment using a structure-based virtual screening approach, Bioorganic Chemistry 121:105675 (2022); Goh et al., SB1317, a potent and orally active FLT3-CDK inhibitor with high anti-tumor efficacy in models of hematological malignancies, Blood 110 (11): 1593 (2007); Dayal et al., Dual FLT3/TOPK inhibitor with activity against FLT3-ITD secondary mutations potently inhibits acute myeloid leukemia cell lines, Future Medicinal Chemistry 10 (7): 823-835 (2018); Czardybon et al., A novel, dual pan-PIM/FLT3 inhibitor SEL24 exhibits broad therapeutic potential in acute myeloid leukemia, Oncotarget 9 (24): 16917-16931 (2018); Taussig et al., EP0042, a dual FLT3 and Aurora kinase inhibitor: preliminary results of an ongoing phase I/IIa first in human study in patients with relapsed/refractory acute myeloid leukemia, Blood 140 (Supplement 1): 6222-6223 (2022). For example, CCT245718, a dual FLT3/Aurora A inhibitor may overcome FLT3-D835Y-mediated resistance. Tariq et al., CCT245718, a dual FLT3/Aurora A inhibitor overcomes D835Y-mediated resistance to FLT3 inhibitors in acute myeloid leukaemia cells, British J Cancer 125:966-974 (2021). In another interesting report, it was shown that the frequency of resistance to a dual FLT3/CDK4 inhibitor (AMG 925) was less than other FLT3 inhibitors (such as quizartinib) that did not inhibit CDK4. Li et al., Discovery of AMG 925, a FLT3 and CDK4 dual kinase inhibitor with preferential affinity for the activated state of FLT3, J Medicinal Chemistry 57 (8): 3430-3449 (2014).

Despite these dual inhibitors, a dual inhibitor of FLT3 and haspin has not to date been identified. Haspin is a novel target in cancer and is the main kinase that phosphorylates histone H3 at T3. Amoussou et al., Haspin: a promising target for the design of inhibitors as potent anticancer drugs, Drug Discovery Today 23 (2): 409-415 (2018). A recent preliminary report indicated that reduction of haspin via genetic means or inhibition by a compound reduced leukemia proliferation. Leveraging the recent revelation that haspin is over-expressed and plays critical proliferative roles in many cancers, in certain embodiments, optimized 3H-pyrazolo[4,3-f]quinoline-based compounds are provided that exhibit improved activity against both FLT3 and haspin (i.e., a 3H-pyrazolo[4,3-f]quinoline-based dual FLT3/haspin inhibitor).

In certain embodiments, a compound is provided that has the structure for formula I:

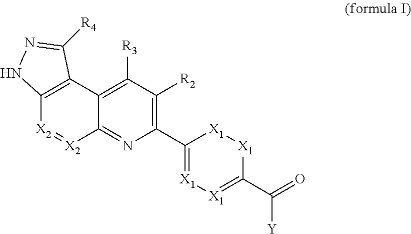

or is a pharmaceutically acceptable salt thereof, wherein:
X₁ is each independently N, CH, or a C-halogen;
X₂ is each independently N, CH, a C-halogen, C—CHₙ₁Xₘ where n1+m=3 and X is a halogen, C—OCF₃, C—OCHF₂, C—OMe, C—Me, C—Et, C-tBu, C-iPr, C—N-alkyl, C—N-heteroalkyl, C—O-alkyl, or C—O-heteroalkyl;
R₂, R₃, and R₄ are each independently an alkyl, cycloalkyl, heteroalkyl, or heterocycloalkyl; and

Y is

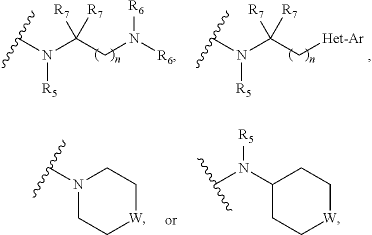

wherein:
n is 1 to 5,
R₅ and R₆ are each independently H, an alkyl, or a heteroalkyl, and R₆ can optionally form a cyclic structure,
R₇ is each independently H or an alkyl,
Het-Ar is a heteroaromatic ring, and
W is NH, NMe, NEt, NCH₂CH₂OH, NCH₂CH₂OMe, O, SO, or SO₂.
   In certain embodiments, Y is not NH₂ (i.e., the compound of formula I, with the proviso that Y is not NH₂).

At least one of X₁ and/or X₂ can be CF, CHF₂, CF₃, or CHF₃. In certain embodiments, each X₂ is CH. In certain embodiments, at least one X₂ is CH. In certain embodiments, each X₁ is CH. In certain embodiments, at least one X₁ is CH.

One or more of R₅ and/or R₆ of Y can be Me. R₇ of Y can each independently be H or Me. In certain embodiments where Y is

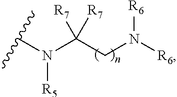

each R₆, taken together, can form a morpholine. In certain embodiments where Y is

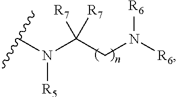

each R₆, taken together, can form a piperdine. In certain embodiments where Y is

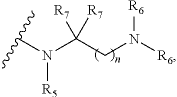

each R₆, taken together, can form a piperazine. In certain embodiments where Y is

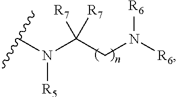

each R₆, taken together, can form a pyrrolidine.

In certain embodiments where Y is

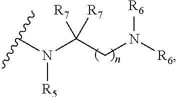

each R₇, when taken together, can form a cyclic structure. The cyclic structure can comprise, for example, a cyclopropyl. The cyclic structure can comprise a cyclobutyl. The cyclic structure can comprise a cyclopentyl. The cyclic structure can comprise a cyclohexyl.

In certain embodiments, where Y is

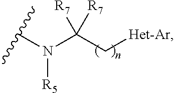

each R₇, when taken together, can form a cyclic structure. The cyclic structure formed by each R₇ can comprise, for example, a cyclopropyl. The cyclic structure can comprise a cyclobutyl. The cyclic structure can comprise a cyclopentyl. The cyclic structure can comprise a cyclohexyl.

In certain embodiments, Y is

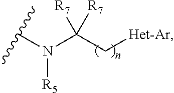

and the Het-Ar comprises an imidazole, an oxazole, a pyrazole, a triazole, an isoxazole, an isothiazole, a tetrazole, an oxadiazole, a thiadiazole, a pyrimidine, or a triazine.

In certain embodiments, Y is s

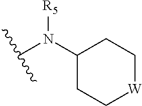

### and R₅ is Me.

In certain embodiments, the compound has a structure of formula IA:

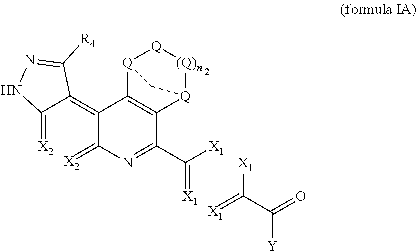

or is a pharmaceutically acceptable salt thereof, wherein:
X₁ is each independently N, CH, or a C-halogen;
X₂ is each independently N, CH, a C-halogen, C—CHₙ₁Xₘ where n1+m=3 and X is a halogen, C—OCF₃, C—OCHF₂, C—OMe, C—Me, C—Et, C-tBu, C-iPr, C—N-alkyl, C—N-heteroalkyl, C—O-alkyl, or C—O-heteroalkyl;
R₄ is an alkyl, cycloalkyl, heteroalkyl, or heterocycloalkyl;
Y is

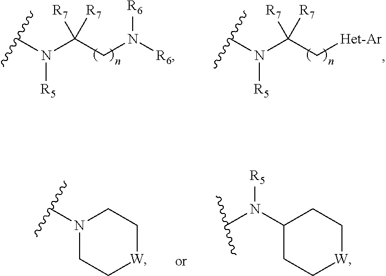

wherein:
n is 1 to 5,
R₅ and R₆ are each independently H, an alkyl, or a heteroalkyl, and R₆ can optionally form a cyclic structure,
R₇ is each independently H or an alkyl,
Het-Ar is a heteroaromatic ring, and
W is NH, NMe, NEt, NCH₂CH₂OH, NCH₂CH₂OMe, O, SO, or SO₂,
with the proviso that Y is not NH₂.
n2 is 1, 2 or 3;
Q is each independently O, NH, substituted N, CH₂, or substituted C, S, SO, or SO₂; and
each Q, taken together, form a 6-, 7-, or 8-membered ring or bicyclic ring.

The compound can have a structure of formula IB:

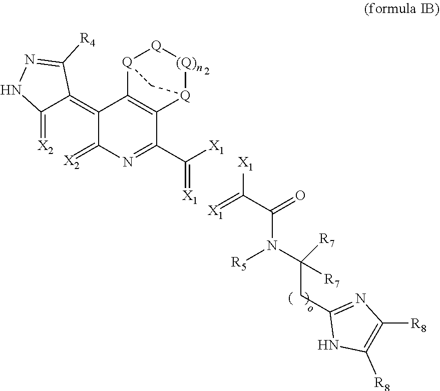

or is a pharmaceutically acceptable salt thereof, wherein:
X₁ is each independently N, CH, or a C-halogen;
X₂ is each independently N, CH, a C-halogen, C—CHₙ₁Xₘ where n1+m=3 and X is a halogen, C—OCF₃, C—OCHF₂, C—OMe, C—Me, C—Et, C-tBu, C-iPr, C—N-alkyl, C—N-heteroalkyl, C—O-alkyl, or C—O-heteroalkyl;
R₄ is an alkyl, cycloalkyl, heteroalkyl, or heterocycloalkyl;
R₅ is H, an alkyl, or a heteroalkyl;
R₇ is each independently H or an alkyl;
o is 0-2; and
each R₈ is independently H, an alkyl, or a heteroalkyl.

In certain embodiments where the compound has a structure of formula IB, each R₇, taken together, forms a cyclic structure. The cyclic structure can comprise a cyclopropyl, a cyclobutyl, a cyclopentyl, or a cyclohexyl. In certain embodiments, at least one R₈ is Me, Et or CF₃.

In certain embodiments, the compound has a structure of:

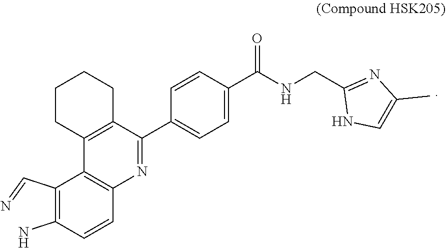 [SMILES: Cc1c[nH]c(CNC(=O)c2ccc(-c3nc4ccc(N)c(C=N)c4c4c3CCCC4)cc2)n1]

The compound can have a structure of, or is a pharmaceutically acceptable salt of, one of the following:

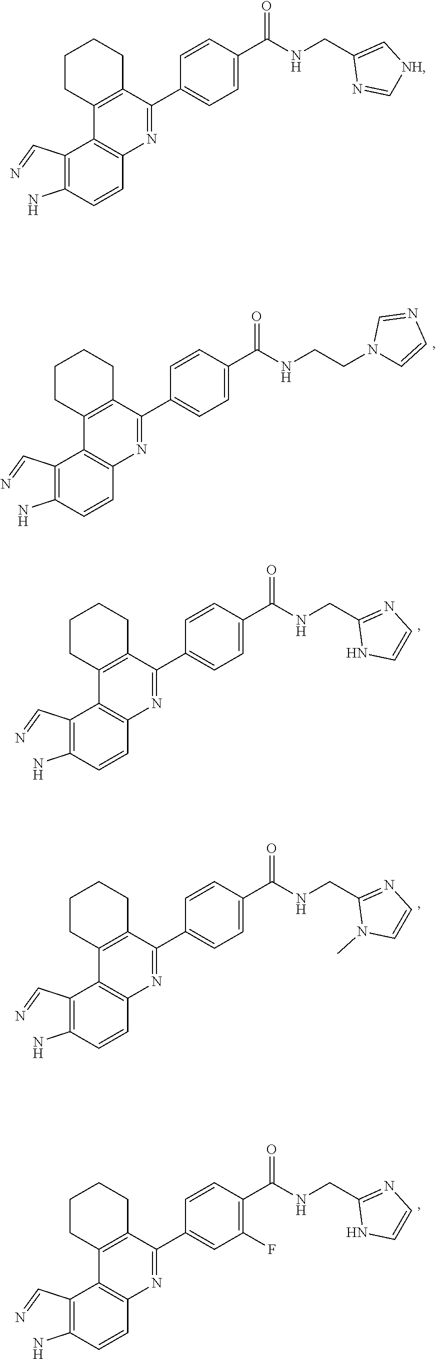 [SMILES: N=Cc1c(N)ccc2nc(-c3ccc(C(=O)NCc4c[nH]cn4)cc3)c3c(c12)CCCC3 ; N=Cc1c(N)ccc2nc(-c3ccc(C(=O)NCCn4ccnc4)cc3)c3c(c12)CCCC3 ; N=Cc1c(N)ccc2nc(-c3ccc(C(=O)NCc4ncc[nH]4)cc3)c3c(c12)CCCC3 ; Cn1ccnc1CNC(=O)c1ccc(-c2nc3ccc(N)c(C=N)c3c3c2CCCC3)cc1 ; N=Cc1c(N)ccc2nc(-c3ccc(C(=O)NCc4ncc[nH]4)c(F)c3)c3c(c12)CCCC3]

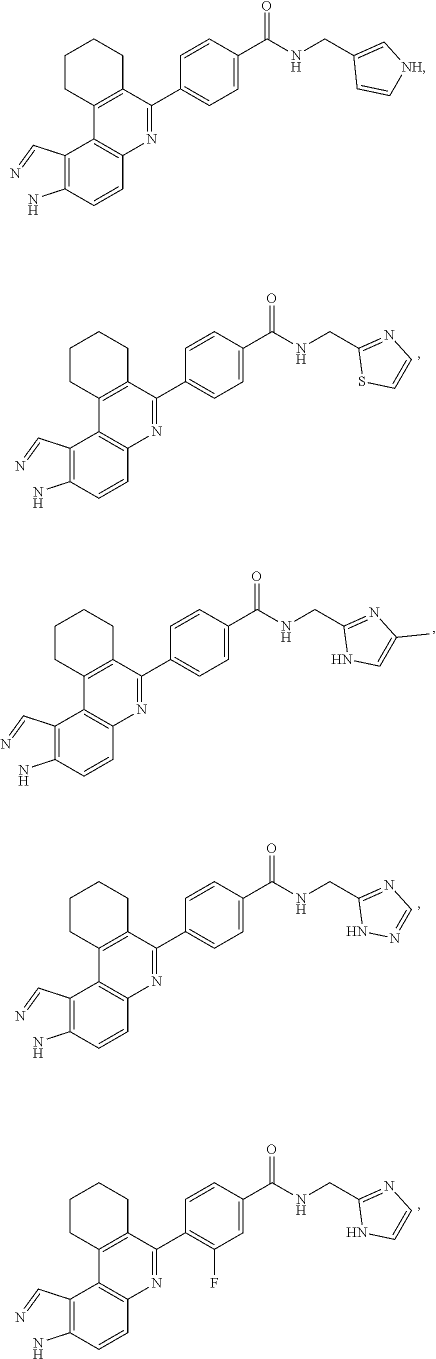 [SMILES: N=Cc1c(N)ccc2nc(-c3ccc(C(=O)NCc4cc[nH]c4)cc3)c3c(c12)CCCC3 ; N=Cc1c(N)ccc2nc(-c3ccc(C(=O)NCc4nccs4)cc3)c3c(c12)CCCC3 ; Cc1c[nH]c(CNC(=O)c2ccc(-c3nc4ccc(N)c(C=N)c4c4c3CCCC4)cc2)n1 ; N=Cc1c(N)ccc2nc(-c3ccc(C(=O)NCc4ncn[nH]4)cc3)c3c(c12)CCCC3 ; N=Cc1c(N)ccc2nc(-c3ccc(C(=O)NCc4ncc[nH]4)cc3F)c3c(c12)CCCC3]

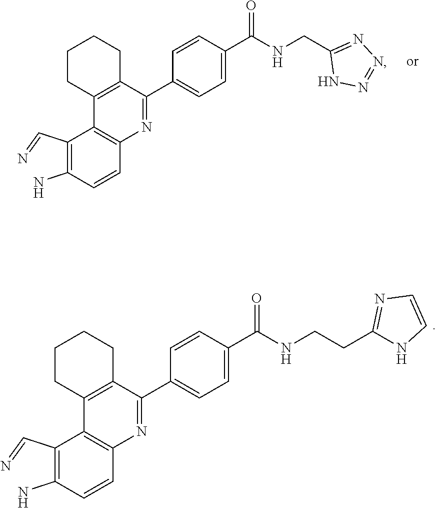 [SMILES: N=Cc1c(N)ccc2nc(-c3ccc(C(=O)NCc4nnn[nH]4)cc3)c3c(c12)CCCC3 ; N=Cc1c(N)ccc2nc(-c3ccc(C(=O)NCCc4ncc[nH]4)cc3)c3c(c12)CCCC3]

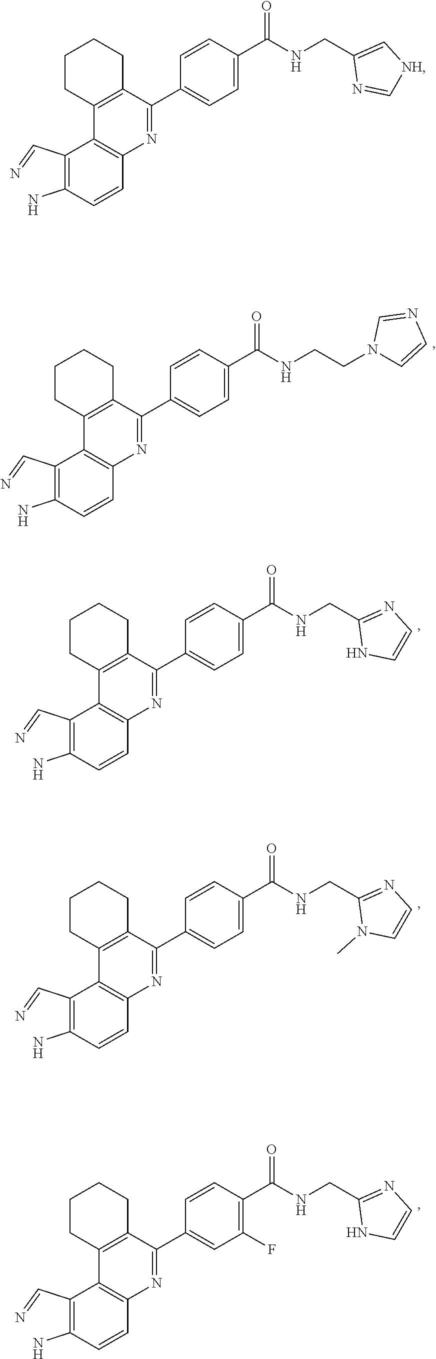 [SMILES: N=Cc1c(N)ccc2nc(-c3ccc(C(=O)NCc4c[nH]cn4)cc3)c3c(c12)CCCC3 ; N=Cc1c(N)ccc2nc(-c3ccc(C(=O)NCCn4ccnc4)cc3)c3c(c12)CCCC3 ; N=Cc1c(N)ccc2nc(-c3ccc(C(=O)NCc4ncc[nH]4)cc3)c3c(c12)CCCC3 ; Cn1ccnc1CNC(=O)c1ccc(-c2nc3ccc(N)c(C=N)c3c3c2CCCC3)cc1 ; N=Cc1c(N)ccc2nc(-c3ccc(C(=O)NCc4ncc[nH]4)c(F)c3)c3c(c12)CCCC3]

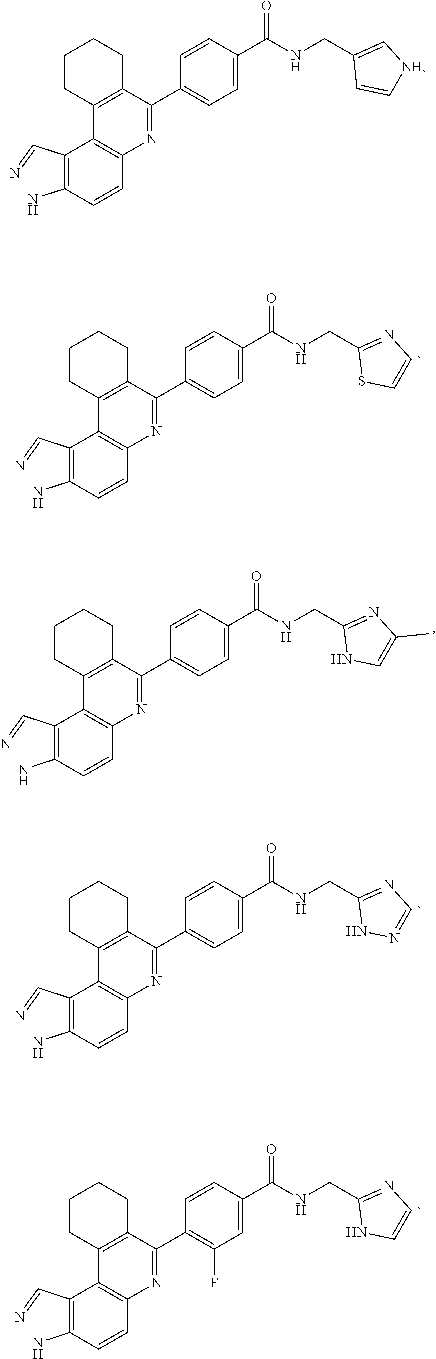 [SMILES: N=Cc1c(N)ccc2nc(-c3ccc(C(=O)NCc4cc[nH]c4)cc3)c3c(c12)CCCC3 ; N=Cc1c(N)ccc2nc(-c3ccc(C(=O)NCc4nccs4)cc3)c3c(c12)CCCC3 ; Cc1c[nH]c(CNC(=O)c2ccc(-c3nc4ccc(N)c(C=N)c4c4c3CCCC4)cc2)n1 ; N=Cc1c(N)ccc2nc(-c3ccc(C(=O)NCc4ncn[nH]4)cc3)c3c(c12)CCCC3 ; N=Cc1c(N)ccc2nc(-c3ccc(C(=O)NCc4ncc[nH]4)cc3F)c3c(c12)CCCC3]

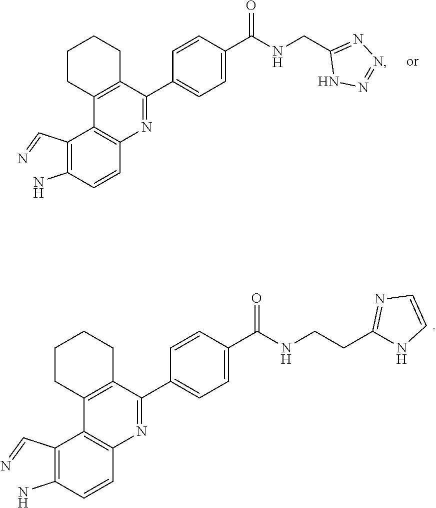 [SMILES: N=Cc1c(N)ccc2nc(-c3ccc(C(=O)NCc4nnn[nH]4)cc3)c3c(c12)CCCC3 ; N=Cc1c(N)ccc2nc(-c3ccc(C(=O)NCCc4ncc[nH]4)cc3)c3c(c12)CCCC3]

In certain embodiments, the compound has a structure of, or is a pharmaceutically acceptable salt of, one of the following:

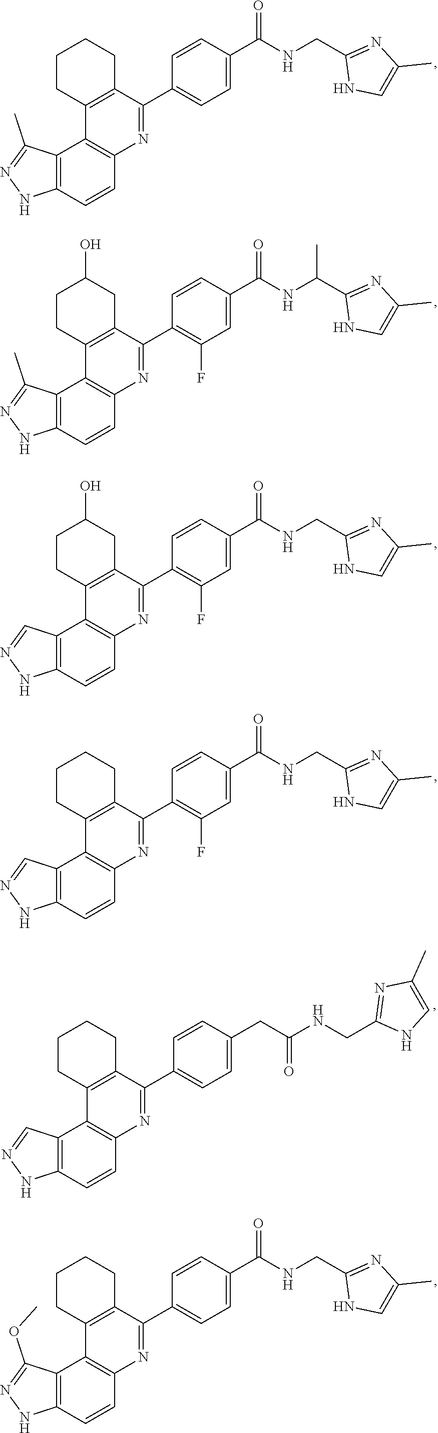

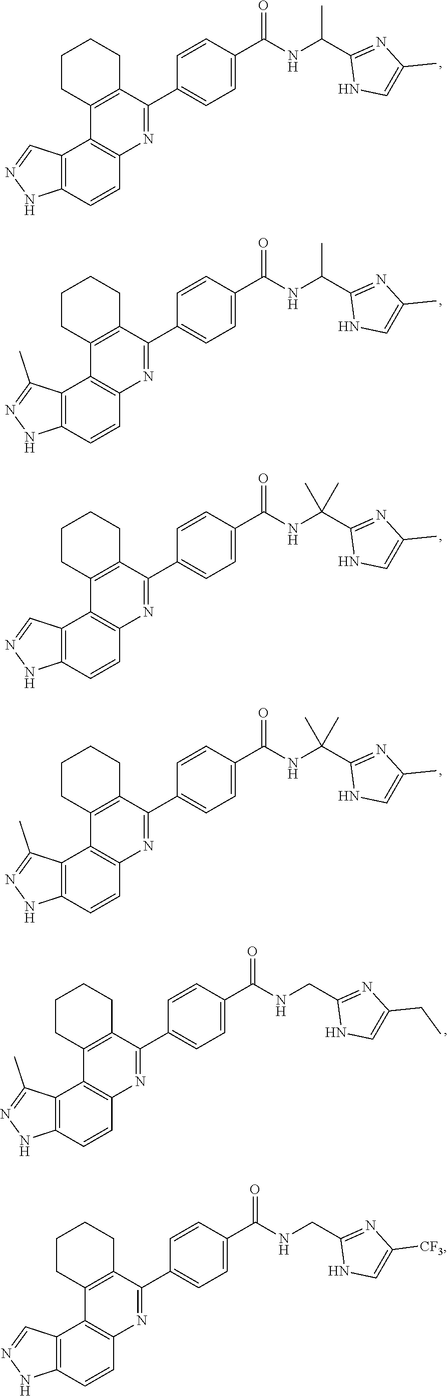 [SMILES: Cc1c[nH]c(C(C)NC(=O)c2ccc(-c3nc4ccc5[nH]ncc5c4c4c3CCCC4)cc2)n1 ; Cc1c[nH]c(C(C)NC(=O)c2ccc(-c3nc4ccc5[nH]nc(C)c5c4c4c3CCCC4)cc2)n1 ; Cc1c[nH]c(C(C)(C)NC(=O)c2ccc(-c3nc4ccc5[nH]ncc5c4c4c3CCCC4)cc2)n1 ; Cc1c[nH]c(C(C)(C)NC(=O)c2ccc(-c3nc4ccc5[nH]nc(C)c5c4c4c3CCCC4)cc2)n1 ; CCc1c[nH]c(CNC(=O)c2ccc(-c3nc4ccc5[nH]nc(C)c5c4c4c3CCCC4)cc2)n1 ; O=C(NCc1nc(C(F)(F)F)c[nH]1)c1ccc(-c2nc3ccc4[nH]ncc4c3c3c2CCCC3)cc1]

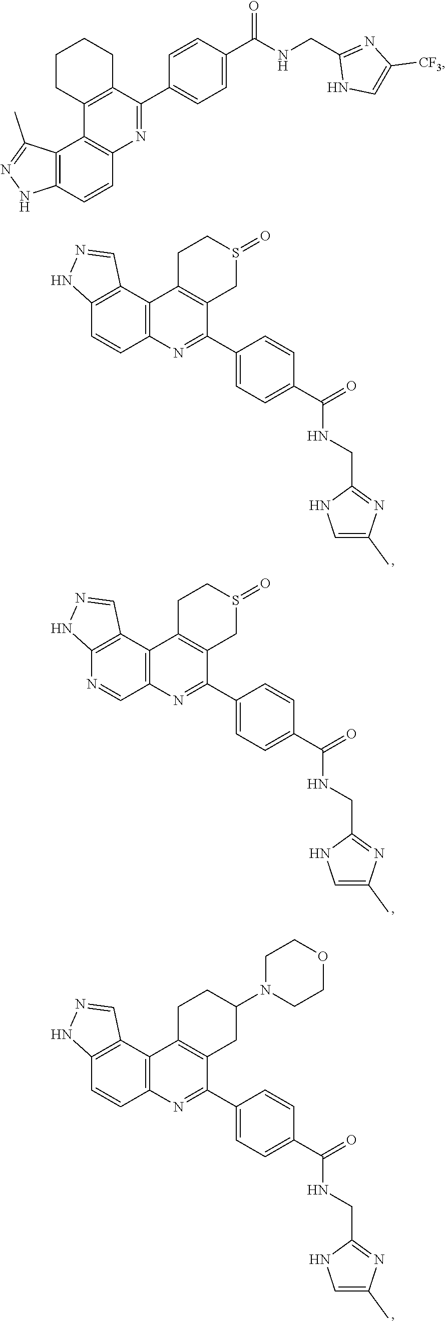 [SMILES: Cc1n[nH]c2ccc3nc(-c4ccc(C(=O)NCc5nc(C(F)(F)F)c[nH]5)cc4)c4c(c3c12)CCCC4 ; Cc1c[nH]c(CNC(=O)c2ccc(-c3nc4ccc5[nH]ncc5c4c4c3CS(=O)CC4)cc2)n1 ; Cc1c[nH]c(CNC(=O)c2ccc(-c3nc4cnc5[nH]ncc5c4c4c3CS(=O)CC4)cc2)n1 ; Cc1c[nH]c(CNC(=O)c2ccc(-c3nc4ccc5[nH]ncc5c4c4c3CC(N3CCOCC3)CC4)cc2)n1]

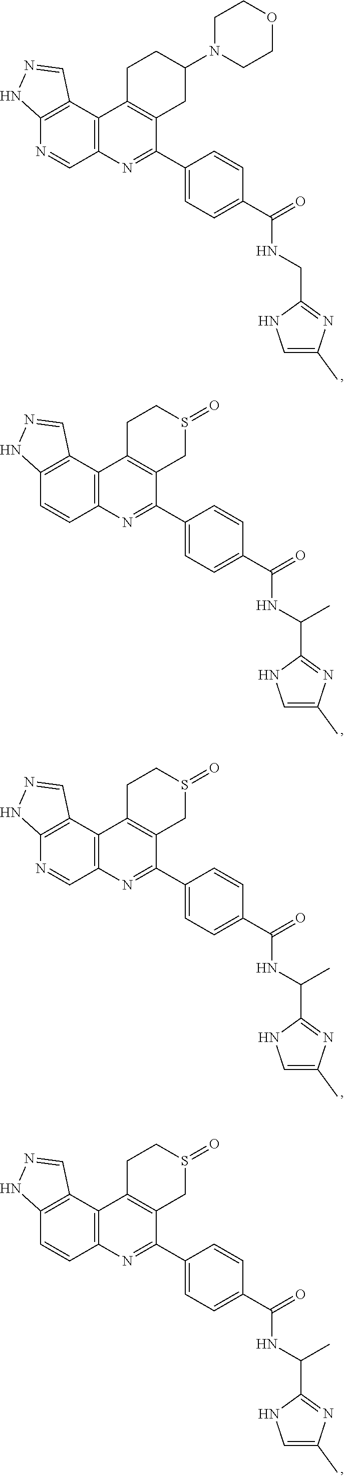 [SMILES: Cc1c[nH]c(CNC(=O)c2ccc(-c3nc4cnc5[nH]ncc5c4c4c3CC(N3CCOCC3)CC4)cc2)n1 ; Cc1c[nH]c(C(C)NC(=O)c2ccc(-c3nc4ccc5[nH]ncc5c4c4c3CS(=O)CC4)cc2)n1 ; Cc1c[nH]c(C(C)NC(=O)c2ccc(-c3nc4cnc5[nH]ncc5c4c4c3CS(=O)CC4)cc2)n1 ; Cc1c[nH]c(C(C)NC(=O)c2ccc(-c3nc4ccc5[nH]ncc5c4c4c3CS(=O)CC4)cc2)n1]

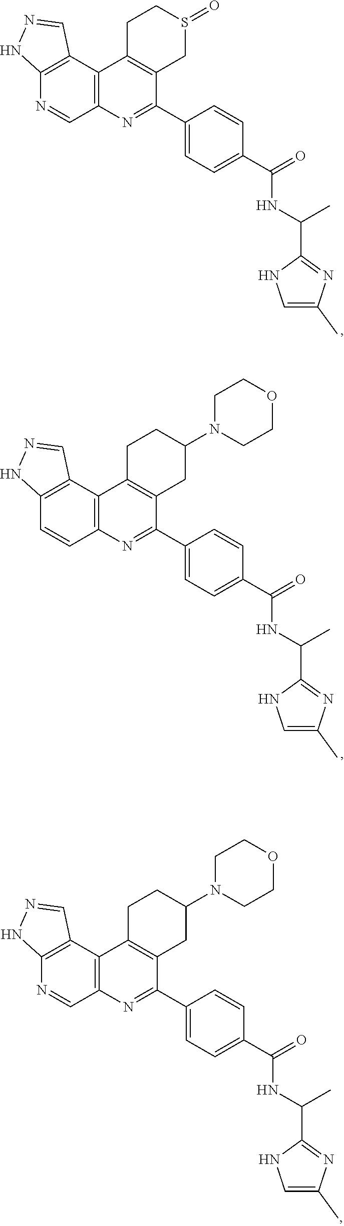 [SMILES: Cc1c[nH]c(C(C)NC(=O)c2ccc(-c3nc4cnc5[nH]ncc5c4c4c3CS(=O)CC4)cc2)n1 ; Cc1c[nH]c(C(C)NC(=O)c2ccc(-c3nc4ccc5[nH]ncc5c4c4c3CC(N3CCOCC3)CC4)cc2)n1 ; Cc1c[nH]c(C(C)NC(=O)c2ccc(-c3nc4cnc5[nH]ncc5c4c4c3CC(N3CCOCC3)CC4)cc2)n1]

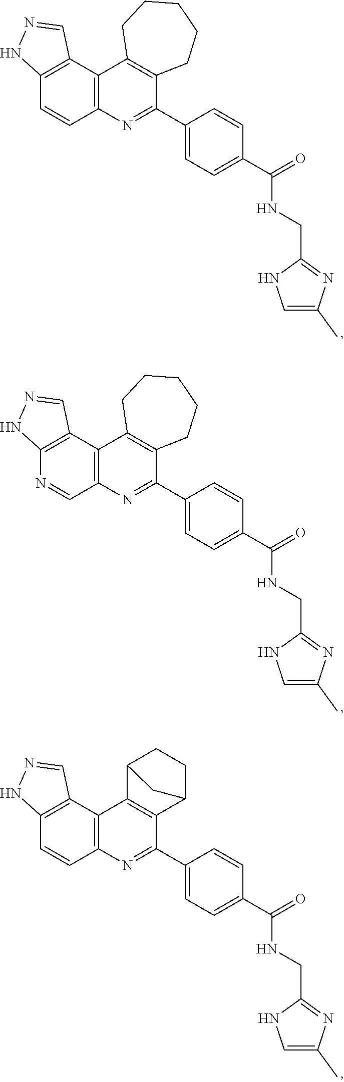 [SMILES: Cc1c[nH]c(CNC(=O)c2ccc(-c3nc4ccc5[nH]ncc5c4c4c3CCCCC4)cc2)n1 ; Cc1c[nH]c(CNC(=O)c2ccc(-c3nc4cnc5[nH]ncc5c4c4c3CCCCC4)cc2)n1 ; Cc1c[nH]c(CNC(=O)c2ccc(-c3nc4ccc5[nH]ncc5c4c4c3C3CCC4C3)cc2)n1]

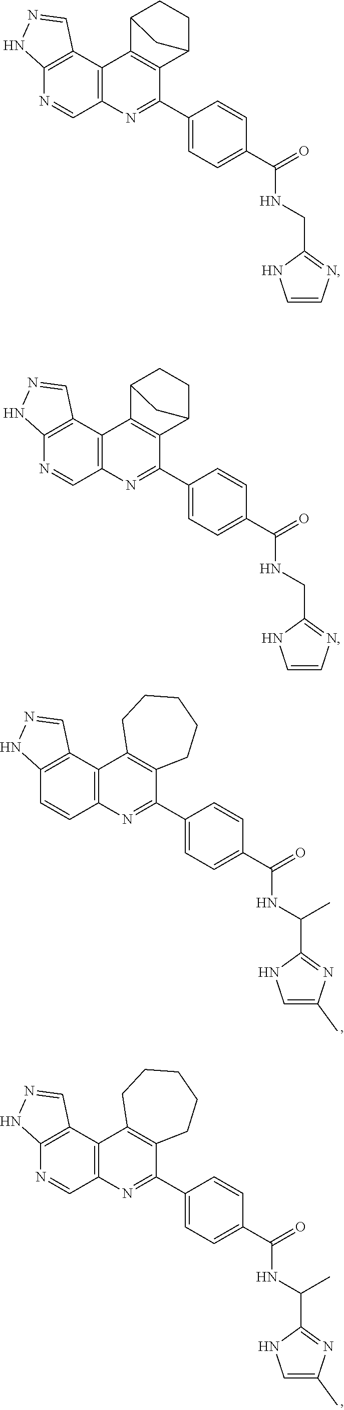 [SMILES: O=C(NCc1ncc[nH]1)c1ccc(-c2nc3cnc4[nH]ncc4c3c3c2C2CCC3C2)cc1 ; O=C(NCc1ncc[nH]1)c1ccc(-c2nc3cnc4[nH]ncc4c3c3c2C2CCC3C2)cc1 ; Cc1c[nH]c(C(C)NC(=O)c2ccc(-c3nc4ccc5[nH]ncc5c4c4c3CCCCC4)cc2)n1 ; Cc1c[nH]c(C(C)NC(=O)c2ccc(-c3nc4cnc5[nH]ncc5c4c4c3CCCCC4)cc2)n1]

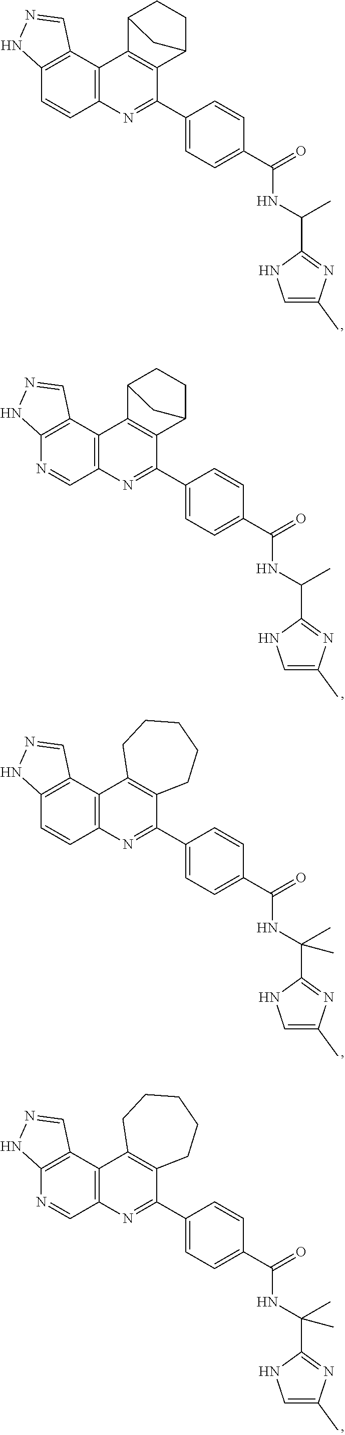 [SMILES: Cc1c[nH]c(C(C)NC(=O)c2ccc(-c3nc4ccc5[nH]ncc5c4c4c3C3CCC4C3)cc2)n1 ; Cc1c[nH]c(C(C)NC(=O)c2ccc(-c3nc4cnc5[nH]ncc5c4c4c3C3CCC4C3)cc2)n1 ; Cc1c[nH]c(C(C)(C)NC(=O)c2ccc(-c3nc4ccc5[nH]ncc5c4c4c3CCCCC4)cc2)n1 ; Cc1c[nH]c(C(C)(C)NC(=O)c2ccc(-c3nc4cnc5[nH]ncc5c4c4c3CCCCC4)cc2)n1]

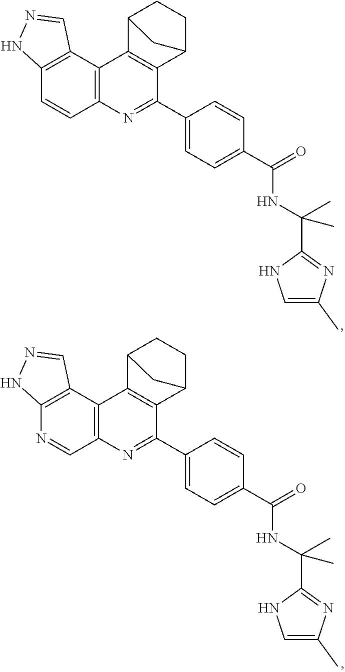 [SMILES: Cc1c[nH]c(C(C)(C)NC(=O)c2ccc(-c3nc4ccc5[nH]ncc5c4c4c3C3CCC4C3)cc2)n1 ; Cc1c[nH]c(C(C)(C)NC(=O)c2ccc(-c3nc4cnc5[nH]ncc5c4c4c3C3CCC4C3)cc2)n1]

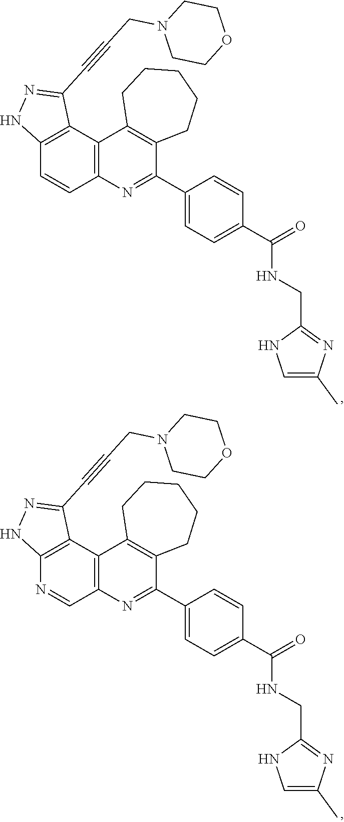 [SMILES: Cc1c[nH]c(CNC(=O)c2ccc(-c3nc4ccc5[nH]nc(C#CCN6CCOCC6)c5c4c4c3CCCCC4)cc2)n1 ; Cc1c[nH]c(CNC(=O)c2ccc(-c3nc4cnc5[nH]nc(C#CCN6CCOCC6)c5c4c4c3CCCCC4)cc2)n1]

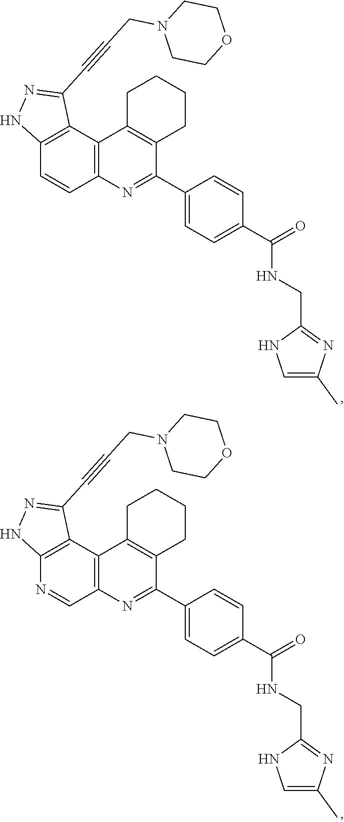 [SMILES: Cc1c[nH]c(CNC(=O)c2ccc(-c3nc4ccc5[nH]nc(C#CCN6CCOCC6)c5c4c4c3CCCC4)cc2)n1 ; Cc1c[nH]c(CNC(=O)c2ccc(-c3nc4cnc5[nH]nc(C#CCN6CCOCC6)c5c4c4c3CCCC4)cc2)n1]

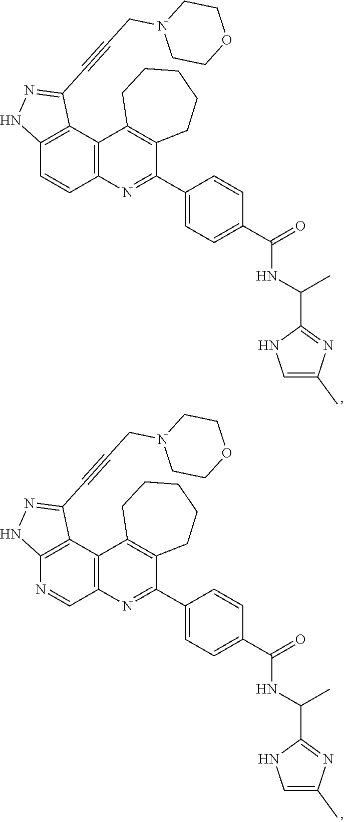 [SMILES: Cc1c[nH]c(C(C)NC(=O)c2ccc(-c3nc4ccc5[nH]nc(C#CCN6CCOCC6)c5c4c4c3CCCCC4)cc2)n1 ; Cc1c[nH]c(C(C)NC(=O)c2ccc(-c3nc4cnc5[nH]nc(C#CCN6CCOCC6)c5c4c4c3CCCCC4)cc2)n1]

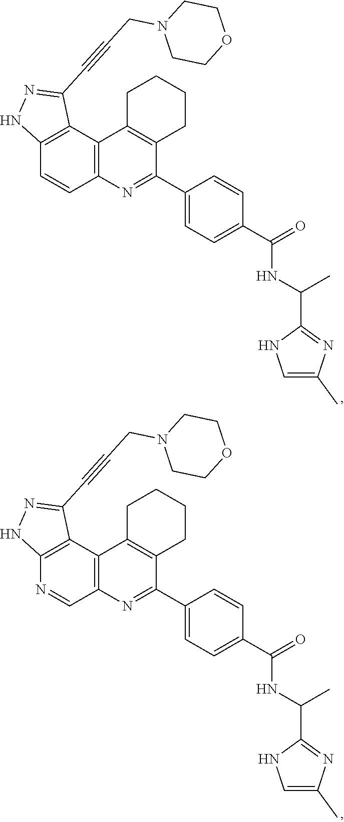 [SMILES: Cc1c[nH]c(C(C)NC(=O)c2ccc(-c3nc4ccc5[nH]nc(C#CCN6CCOCC6)c5c4c4c3CCCC4)cc2)n1 ; Cc1c[nH]c(C(C)NC(=O)c2ccc(-c3nc4cnc5[nH]nc(C#CCN6CCOCC6)c5c4c4c3CCCC4)cc2)n1]

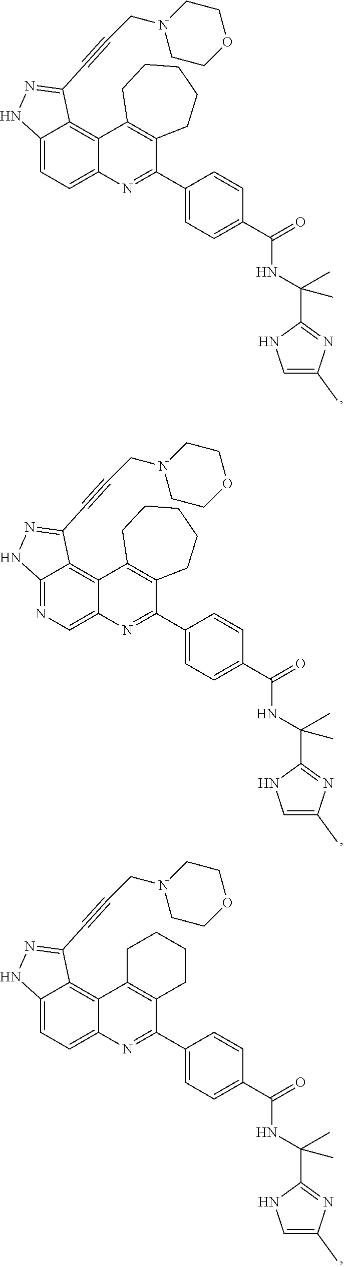 [SMILES: Cc1c[nH]c(C(C)(C)NC(=O)c2ccc(-c3nc4ccc5[nH]nc(C#CCN6CCOCC6)c5c4c4c3CCCCC4)cc2)n1 ; Cc1c[nH]c(C(C)(C)NC(=O)c2ccc(-c3nc4cnc5[nH]nc(C#CCN6CCOCC6)c5c4c4c3CCCCC4)cc2)n1 ; Cc1c[nH]c(C(C)(C)NC(=O)c2ccc(-c3nc4ccc5[nH]nc(C#CCN6CCOCC6)c5c4c4c3CCCC4)cc2)n1]

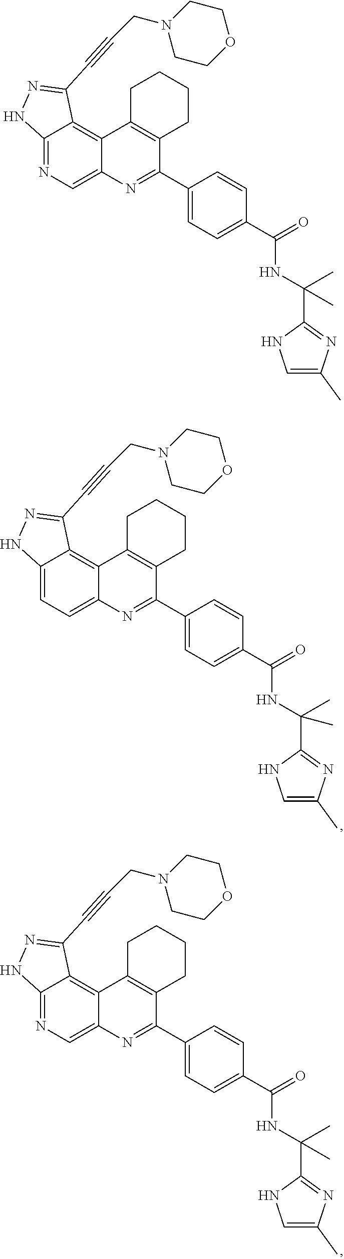 [SMILES: Cc1c[nH]c(C(C)(C)NC(=O)c2ccc(-c3nc4cnc5[nH]nc(C#CCN6CCOCC6)c5c4c4c3CCCC4)cc2)n1 ; Cc1c[nH]c(C(C)(C)NC(=O)c2ccc(-c3nc4ccc5[nH]nc(C#CCN6CCOCC6)c5c4c4c3CCCC4)cc2)n1 ; Cc1c[nH]c(C(C)(C)NC(=O)c2ccc(-c3nc4cnc5[nH]nc(C#CCN6CCOCC6)c5c4c4c3CCCC4)cc2)n1]

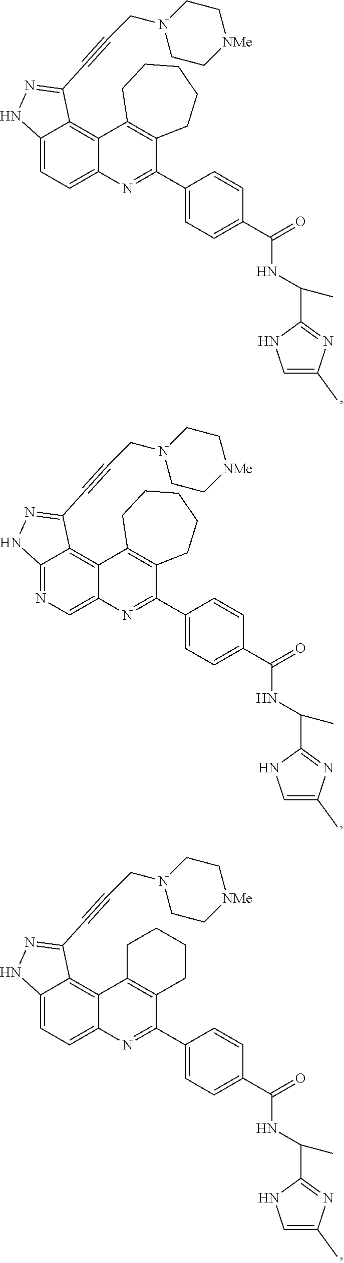 [SMILES: Cc1c[nH]c(C(C)NC(=O)c2ccc(-c3nc4ccc5[nH]nc(C#CCN6CCN(C)CC6)c5c4c4c3CCCCC4)cc2)n1 ; Cc1c[nH]c(C(C)NC(=O)c2ccc(-c3nc4cnc5[nH]nc(C#CCN6CCN(C)CC6)c5c4c4c3CCCCC4)cc2)n1 ; Cc1c[nH]c(C(C)NC(=O)c2ccc(-c3nc4ccc5[nH]nc(C#CCN6CCN(C)CC6)c5c4c4c3CCCC4)cc2)n1]

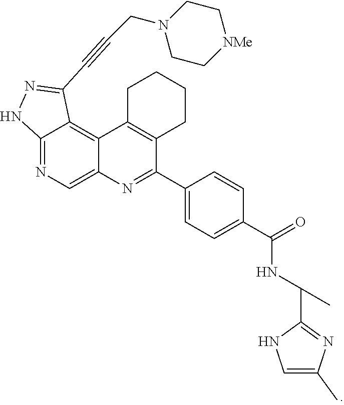 [SMILES: Cc1c[nH]c(C(C)NC(=O)c2ccc(-c3nc4cnc5[nH]nc(C#CCN6CCN(C)CC6)c5c4c4c3CCCC4)cc2)n1]

In certain embodiments, the compound has a structure of, or is a pharmaceutically acceptable salt of, one of the following:

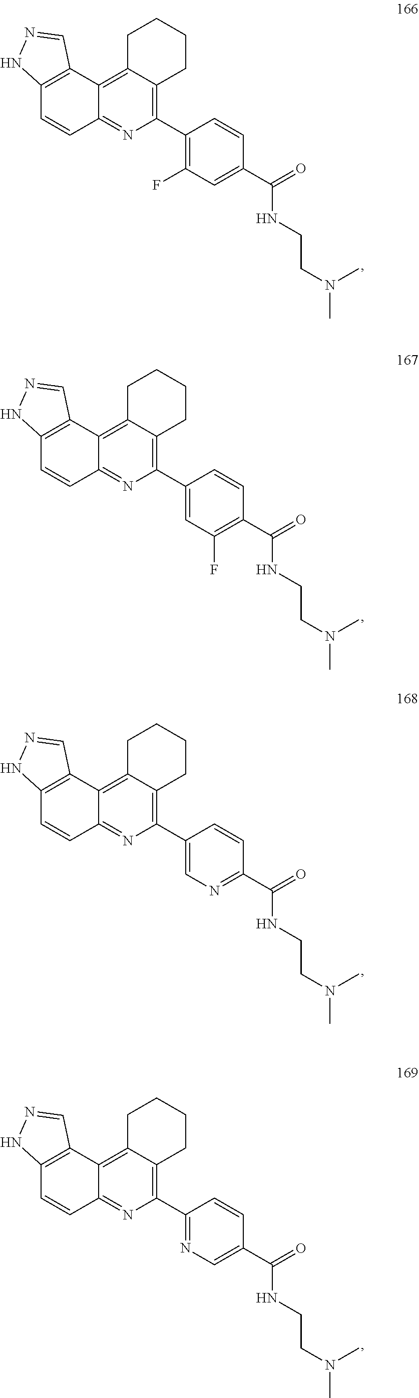 [SMILES: CN(C)CCNC(=O)c1ccc(-c2nc3ccc4[nH]ncc4c3c3c2CCCC3)c(F)c1 ; CN(C)CCNC(=O)c1ccc(-c2nc3ccc4[nH]ncc4c3c3c2CCCC3)cc1F ; CN(C)CCNC(=O)c1ccc(-c2nc3ccc4[nH]ncc4c3c3c2CCCC3)cn1 ; CN(C)CCNC(=O)c1ccc(-c2nc3ccc4[nH]ncc4c3c3c2CCCC3)nc1]

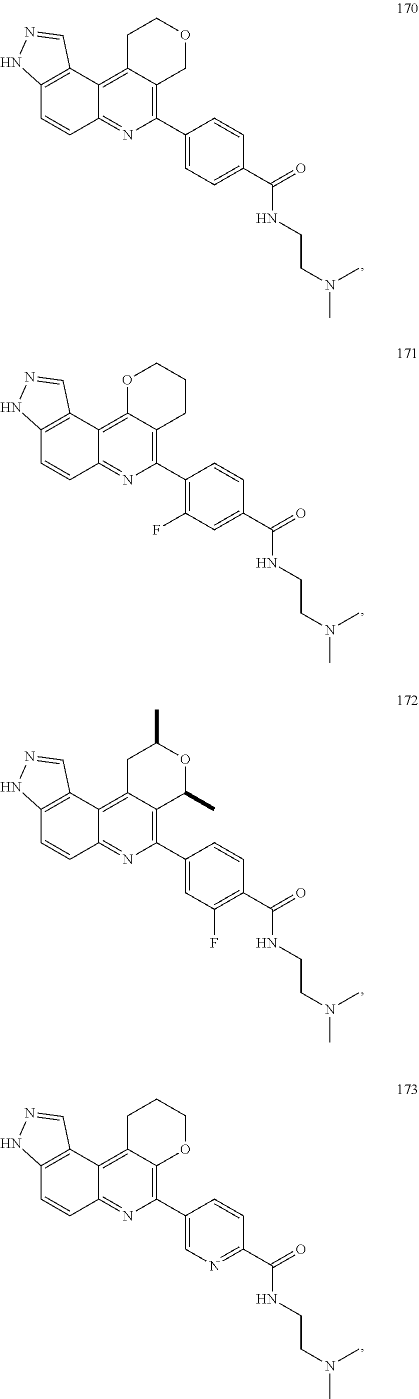 [SMILES: CN(C)CCNC(=O)c1ccc(-c2nc3ccc4[nH]ncc4c3c3c2COCC3)cc1 ; CN(C)CCNC(=O)c1ccc(-c2nc3ccc4[nH]ncc4c3c3c2CCCO3)c(F)c1 ; C[C@@H]1Cc2c(c(-c3ccc(C(=O)NCCN(C)C)c(F)c3)nc3ccc4[nH]ncc4c23)[C@H](C)O1 ; CN(C)CCNC(=O)c1ccc(-c2nc3ccc4[nH]ncc4c3c3c2OCCC3)cn1]

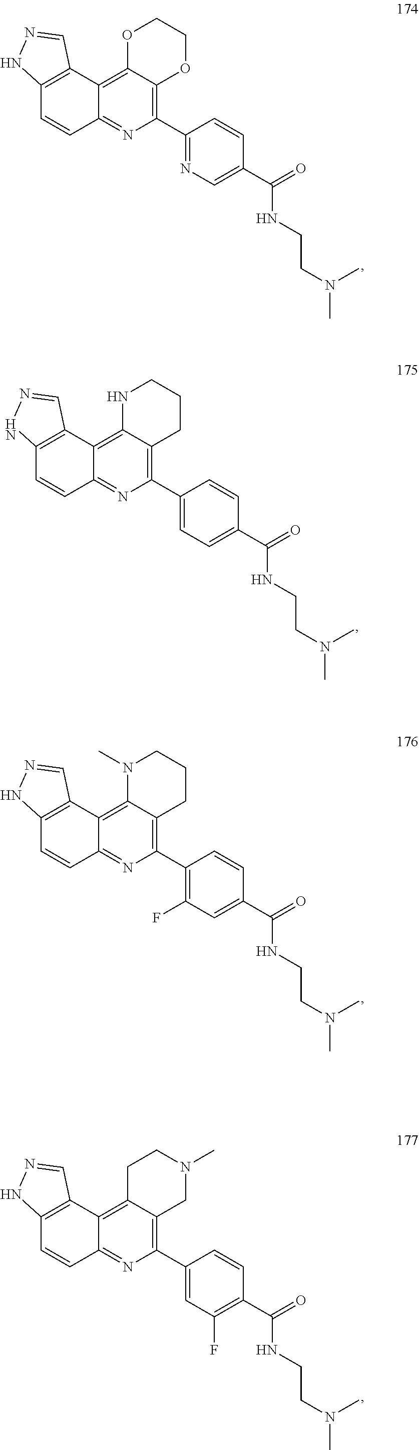 [SMILES: CN(C)CCNC(=O)c1ccc(-c2nc3ccc4[nH]ncc4c3c3c2OCCO3)nc1 ; CN(C)CCNC(=O)c1ccc(-c2nc3ccc4[nH]ncc4c3c3c2CCCN3)cc1 ; CN(C)CCNC(=O)c1ccc(-c2nc3ccc4[nH]ncc4c3c3c2CCCN3C)c(F)c1 ; CN(C)CCNC(=O)c1ccc(-c2nc3ccc4[nH]ncc4c3c3c2CN(C)CC3)cc1F]

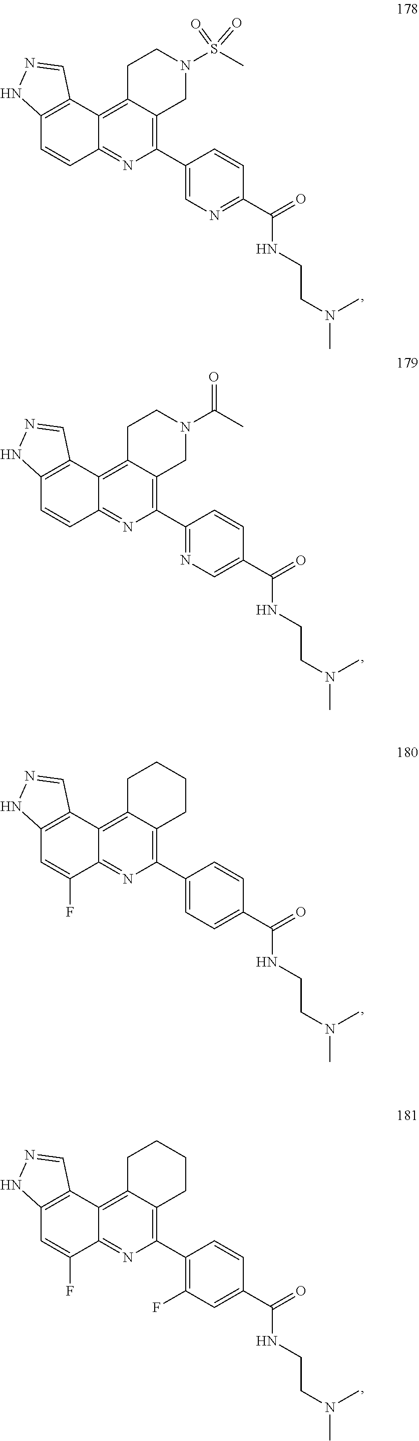 [SMILES: CN(C)CCNC(=O)c1ccc(-c2nc3ccc4[nH]ncc4c3c3c2CN(S(C)(=O)=O)CC3)cn1 ; CC(=O)N1CCc2c(c(-c3ccc(C(=O)NCCN(C)C)cn3)nc3ccc4[nH]ncc4c23)C1 ; CN(C)CCNC(=O)c1ccc(-c2nc3c(F)cc4[nH]ncc4c3c3c2CCCC3)cc1 ; CN(C)CCNC(=O)c1ccc(-c2nc3c(F)cc4[nH]ncc4c3c3c2CCCC3)c(F)c1]

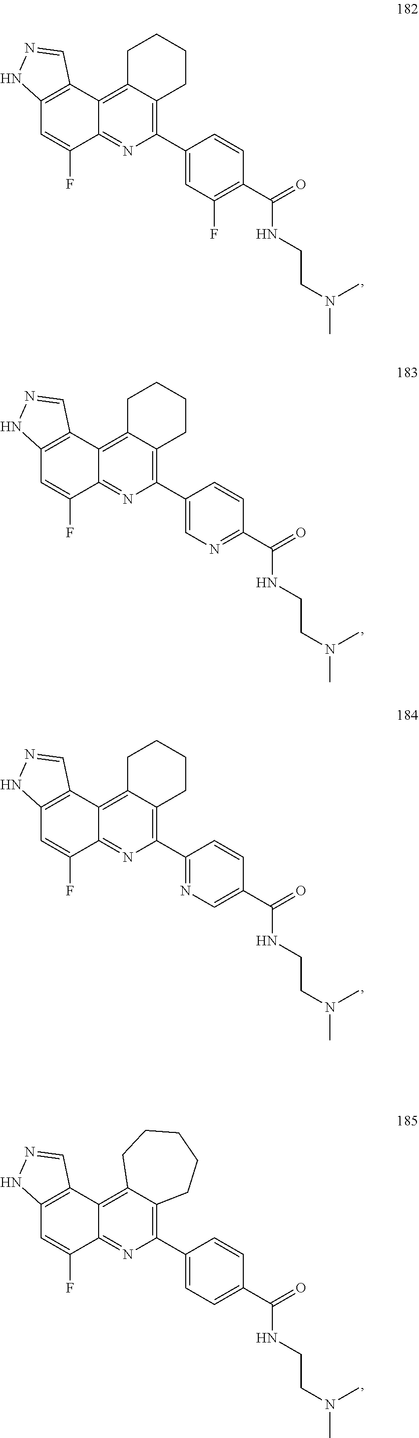 [SMILES: CN(C)CCNC(=O)c1ccc(-c2nc3c(F)cc4[nH]ncc4c3c3c2CCCC3)cc1F ; CN(C)CCNC(=O)c1ccc(-c2nc3c(F)cc4[nH]ncc4c3c3c2CCCC3)cn1 ; CN(C)CCNC(=O)c1ccc(-c2nc3c(F)cc4[nH]ncc4c3c3c2CCCC3)nc1 ; CN(C)CCNC(=O)c1ccc(-c2nc3c(F)cc4[nH]ncc4c3c3c2CCCCC3)cc1]

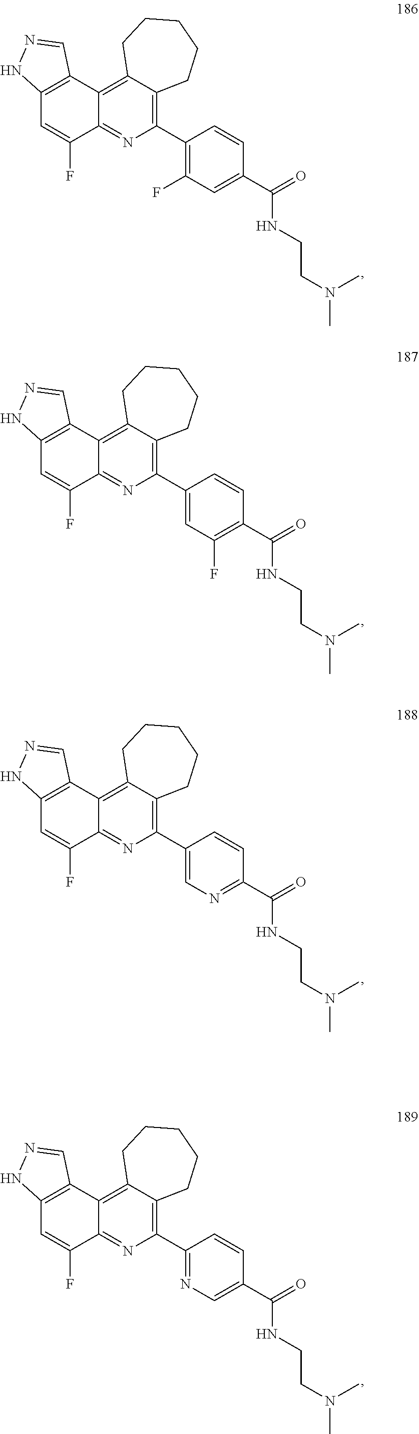 [SMILES: CN(C)CCNC(=O)c1ccc(-c2nc3c(F)cc4[nH]ncc4c3c3c2CCCCC3)c(F)c1 ; CN(C)CCNC(=O)c1ccc(-c2nc3c(F)cc4[nH]ncc4c3c3c2CCCCC3)cc1F ; CN(C)CCNC(=O)c1ccc(-c2nc3c(F)cc4[nH]ncc4c3c3c2CCCCC3)cn1 ; CN(C)CCNC(=O)c1ccc(-c2nc3c(F)cc4[nH]ncc4c3c3c2CCCCC3)nc1]

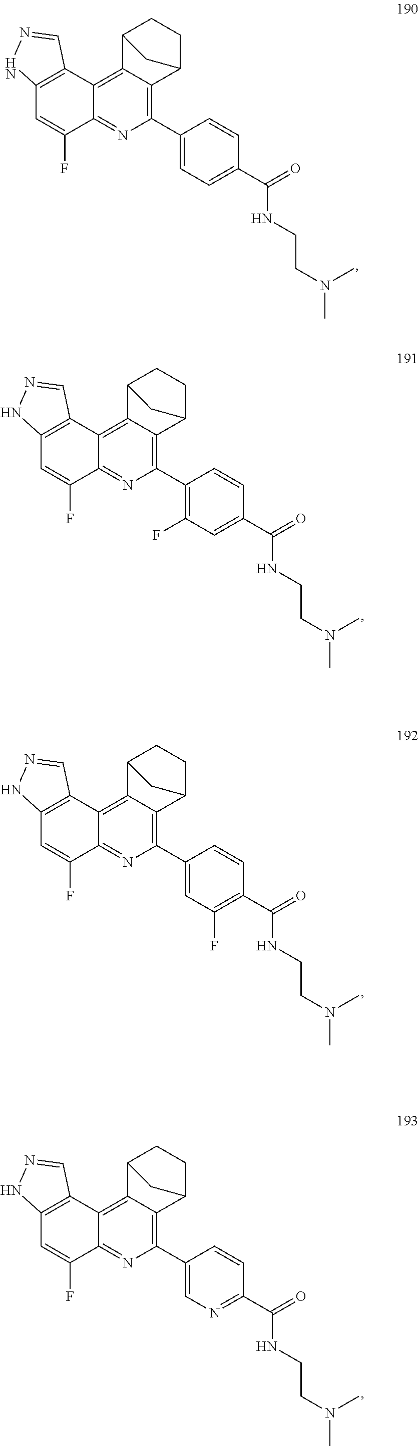 [SMILES: CN(C)CCNC(=O)c1ccc(-c2nc3c(F)cc4[nH]ncc4c3c3c2C2CCC3C2)cc1 ; CN(C)CCNC(=O)c1ccc(-c2nc3c(F)cc4[nH]ncc4c3c3c2C2CCC3C2)c(F)c1 ; CN(C)CCNC(=O)c1ccc(-c2nc3c(F)cc4[nH]ncc4c3c3c2C2CCC3C2)cc1F ; CN(C)CCNC(=O)c1ccc(-c2nc3c(F)cc4[nH]ncc4c3c3c2C2CCC3C2)cn1]

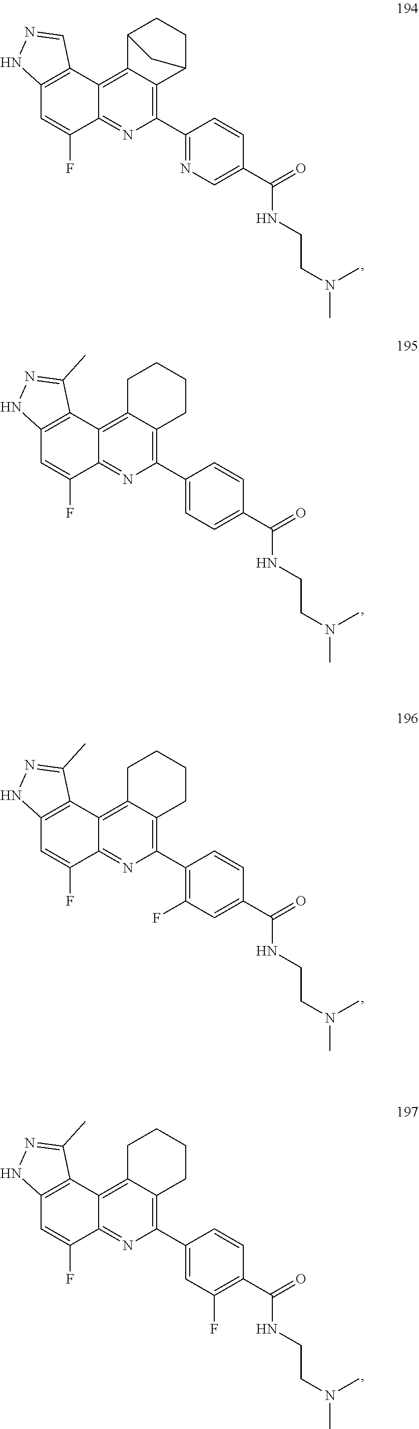 [SMILES: CN(C)CCNC(=O)c1ccc(-c2nc3c(F)cc4[nH]ncc4c3c3c2C2CCC3C2)nc1 ; Cc1n[nH]c2cc(F)c3nc(-c4ccc(C(=O)NCCN(C)C)cc4)c4c(c3c12)CCCC4 ; Cc1n[nH]c2cc(F)c3nc(-c4ccc(C(=O)NCCN(C)C)cc4F)c4c(c3c12)CCCC4 ; Cc1n[nH]c2cc(F)c3nc(-c4ccc(C(=O)NCCN(C)C)c(F)c4)c4c(c3c12)CCCC4]

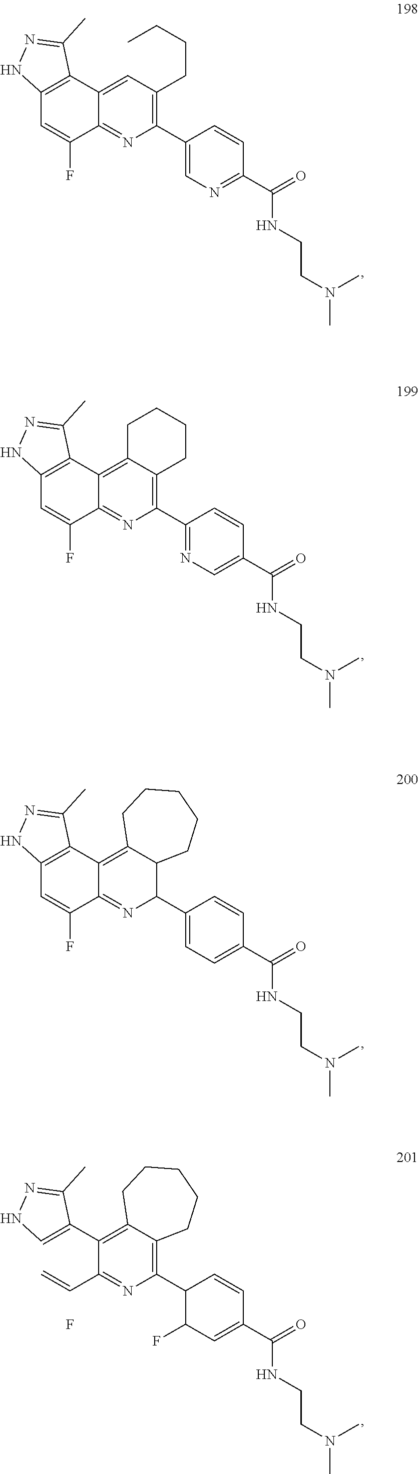

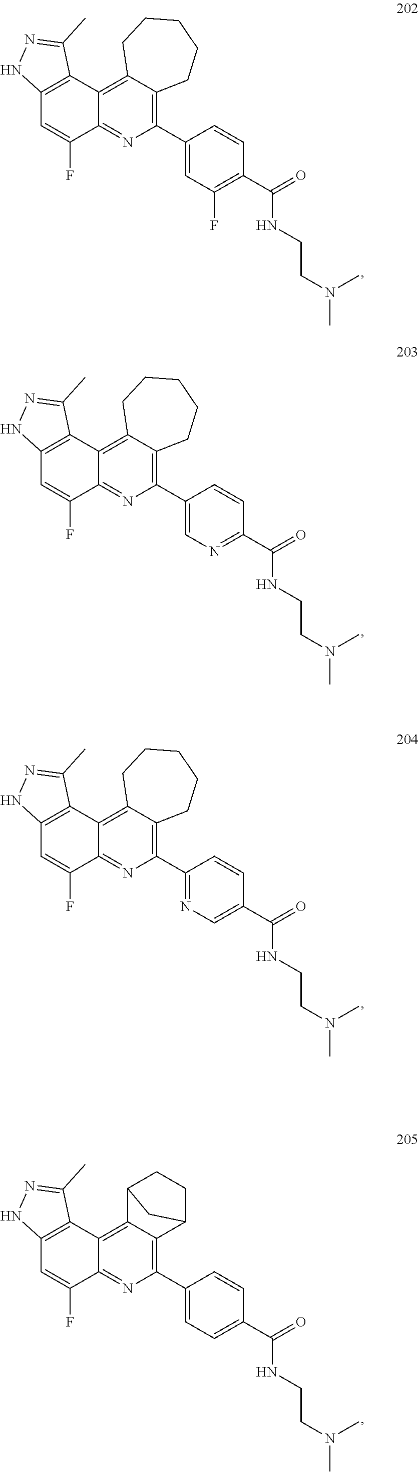 [SMILES: Cc1n[nH]c2cc(F)c3nc(-c4ccc(C(=O)NCCN(C)C)c(F)c4)c4c(c3c12)CCCCC4 ; Cc1n[nH]c2cc(F)c3nc(-c4ccc(C(=O)NCCN(C)C)nc4)c4c(c3c12)CCCCC4 ; Cc1n[nH]c2cc(F)c3nc(-c4ccc(C(=O)NCCN(C)C)cn4)c4c(c3c12)CCCCC4 ; Cc1n[nH]c2cc(F)c3nc(-c4ccc(C(=O)NCCN(C)C)cc4)c4c(c3c12)C1CCC4C1]

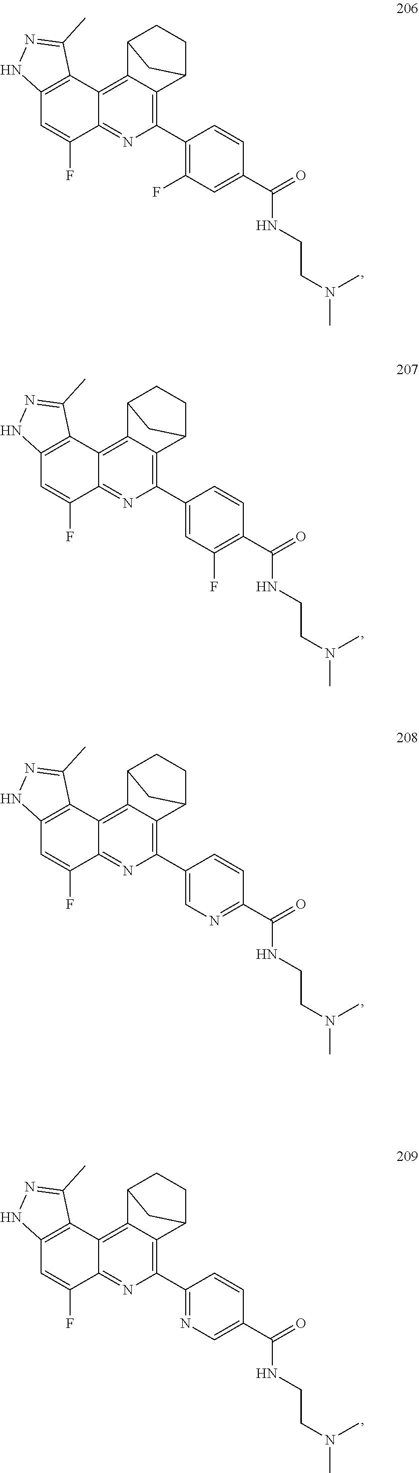 [SMILES: Cc1n[nH]c2cc(F)c3nc(-c4ccc(C(=O)NCCN(C)C)cc4F)c4c(c3c12)C1CCC4C1 ; Cc1n[nH]c2cc(F)c3nc(-c4ccc(C(=O)NCCN(C)C)c(F)c4)c4c(c3c12)C1CCC4C1 ; Cc1n[nH]c2cc(F)c3nc(-c4ccc(C(=O)NCCN(C)C)nc4)c4c(c3c12)C1CCC4C1 ; Cc1n[nH]c2cc(F)c3nc(-c4ccc(C(=O)NCCN(C)C)cn4)c4c(c3c12)C1CCC4C1]

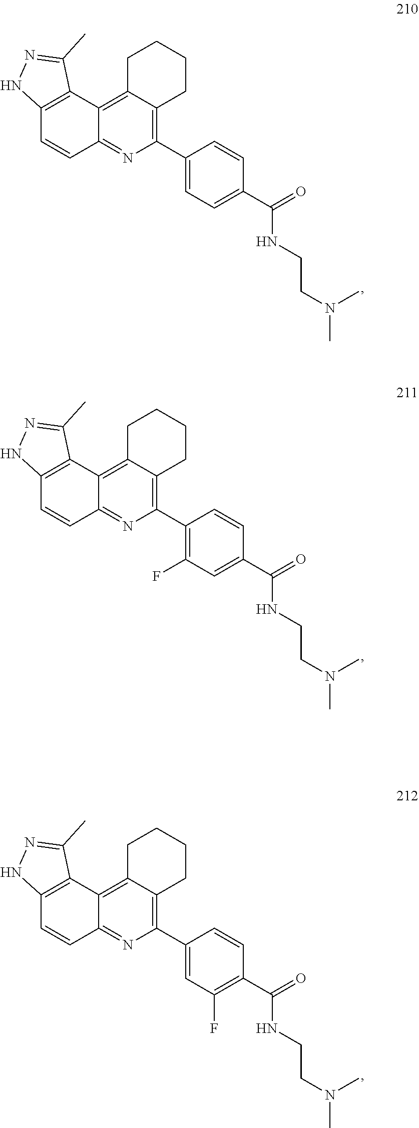 [SMILES: Cc1n[nH]c2ccc3nc(-c4ccc(C(=O)NCCN(C)C)cc4)c4c(c3c12)CCCC4 ; Cc1n[nH]c2ccc3nc(-c4ccc(C(=O)NCCN(C)C)cc4F)c4c(c3c12)CCCC4 ; Cc1n[nH]c2ccc3nc(-c4ccc(C(=O)NCCN(C)C)c(F)c4)c4c(c3c12)CCCC4]

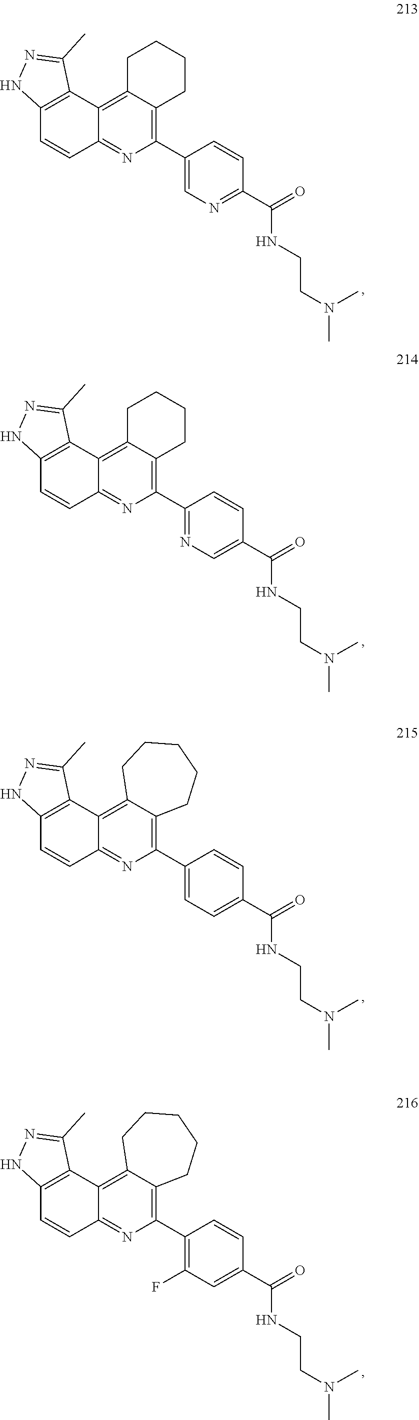 [SMILES: Cc1n[nH]c2ccc3nc(-c4ccc(C(=O)NCCN(C)C)nc4)c4c(c3c12)CCCC4 ; Cc1n[nH]c2ccc3nc(-c4ccc(C(=O)NCCN(C)C)cn4)c4c(c3c12)CCCC4 ; Cc1n[nH]c2ccc3nc(-c4ccc(C(=O)NCCN(C)C)cc4)c4c(c3c12)CCCCC4 ; Cc1n[nH]c2ccc3nc(-c4ccc(C(=O)NCCN(C)C)cc4F)c4c(c3c12)CCCCC4]

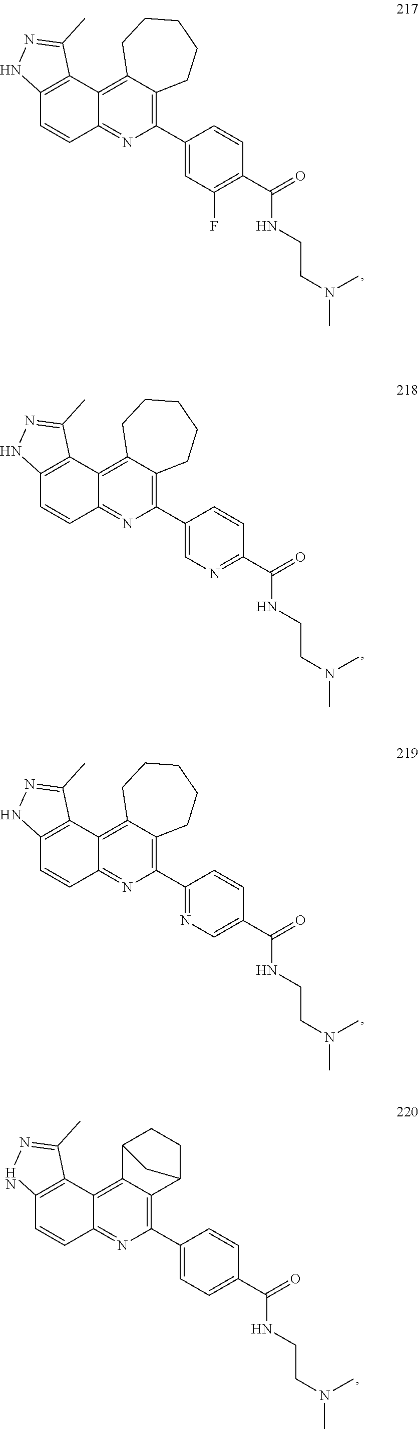 [SMILES: Cc1n[nH]c2ccc3nc(-c4ccc(C(=O)NCCN(C)C)c(F)c4)c4c(c3c12)CCCCC4 ; Cc1n[nH]c2ccc3nc(-c4ccc(C(=O)NCCN(C)C)nc4)c4c(c3c12)CCCCC4 ; Cc1n[nH]c2ccc3nc(-c4ccc(C(=O)NCCN(C)C)cn4)c4c(c3c12)CCCCC4 ; Cc1n[nH]c2ccc3nc(-c4ccc(C(=O)NCCN(C)C)cc4)c4c(c3c12)C1CCC4C1]

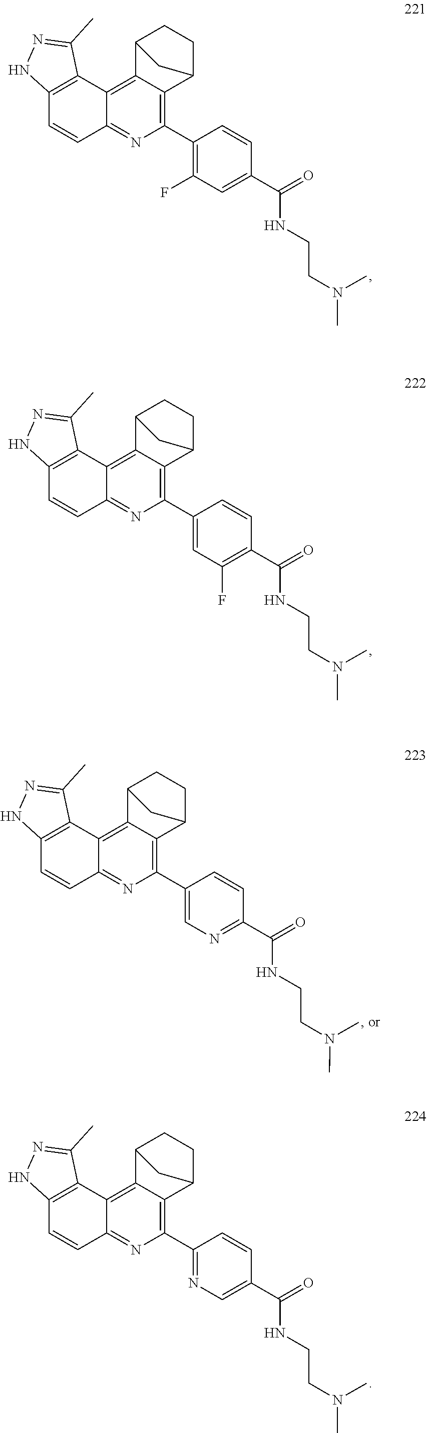 [SMILES: Cc1n[nH]c2ccc3nc(-c4ccc(C(=O)NCCN(C)C)cc4F)c4c(c3c12)C1CCC4C1 ; Cc1n[nH]c2ccc3nc(-c4ccc(C(=O)NCCN(C)C)c(F)c4)c4c(c3c12)C1CCC4C1 ; Cc1n[nH]c2ccc3nc(-c4ccc(C(=O)NCCN(C)C)nc4)c4c(c3c12)C1CCC4C1 ; Cc1n[nH]c2ccc3nc(-c4ccc(C(=O)NCCN(C)C)cn4)c4c(c3c12)C1CCC4C1]

In certain embodiments, the compound has a structure of, or is a pharmaceutically acceptable salt of, one of the following:

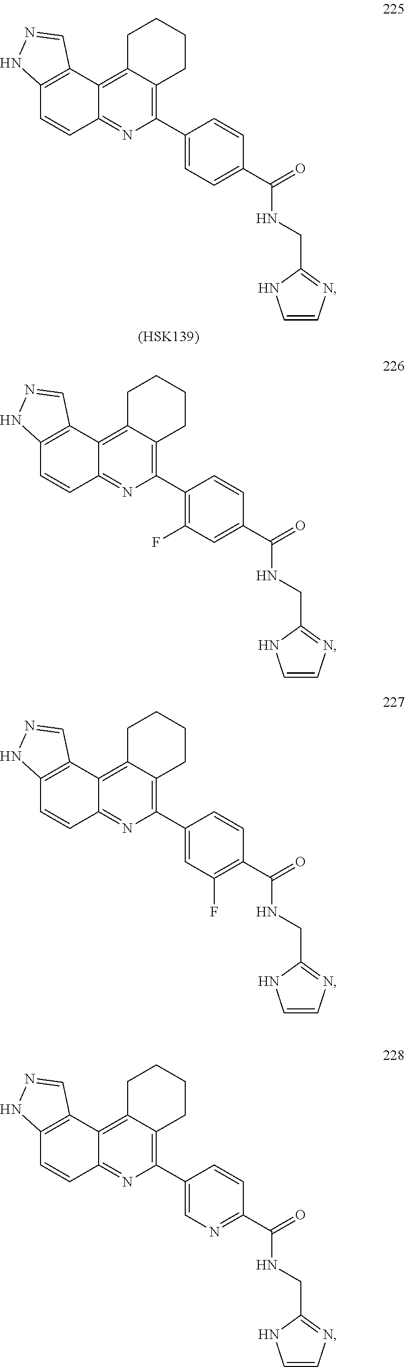 [SMILES: O=C(NCc1ncc[nH]1)c1ccc(-c2nc3ccc4[nH]ncc4c3c3c2CCCC3)c(F)c1 ; O=C(NCc1ncc[nH]1)c1ccc(-c2nc3ccc4[nH]ncc4c3c3c2CCCC3)cc1 ; "(HSK139)" ; O=C(NCc1ncc[nH]1)c1ccc(-c2nc3ccc4[nH]ncc4c3c3c2CCCC3)cc1F ; O=C(NCc1ncc[nH]1)c1ccc(-c2nc3ccc4[nH]ncc4c3c3c2CCCC3)cn1]

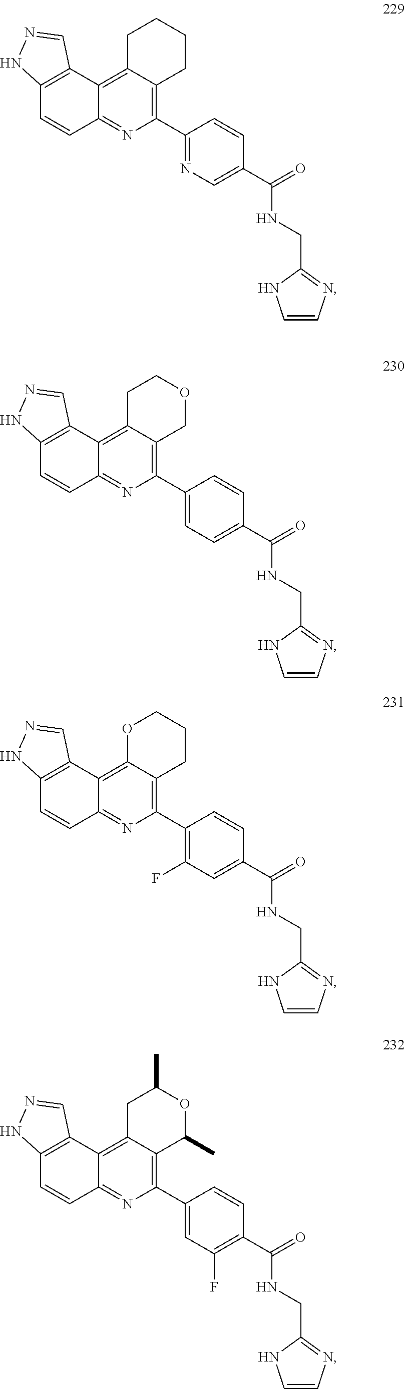 [SMILES: O=C(NCc1ncc[nH]1)c1ccc(-c2nc3ccc4[nH]ncc4c3c3c2CCCC3)nc1 ; O=C(NCc1ncc[nH]1)c1ccc(-c2nc3ccc4[nH]ncc4c3c3c2COCC3)cc1 ; O=C(NCc1ncc[nH]1)c1ccc(-c2nc3ccc4[nH]ncc4c3c3c2CCCO3)c(F)c1 ; C[C@@H]1Cc2c(c(-c3ccc(C(=O)NCc4ncc[nH]4)c(F)c3)nc3ccc4[nH]ncc4c23)[C@H](C)O1]

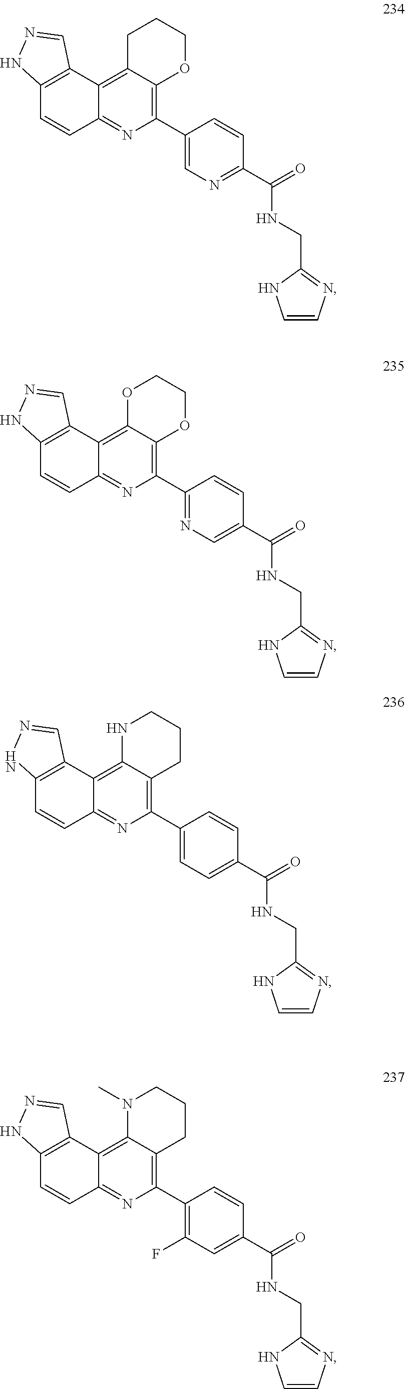 [SMILES: O=C(NCc1ncc[nH]1)c1ccc(-c2nc3ccc4[nH]ncc4c3c3c2OCCC3)cn1 ; O=C(NCc1ncc[nH]1)c1ccc(-c2nc3ccc4[nH]ncc4c3c3c2OCCO3)nc1 ; O=C(NCc1ncc[nH]1)c1ccc(-c2nc3ccc4[nH]ncc4c3c3c2CCCN3)cc1 ; CN1CCCc2c(-c3ccc(C(=O)NCc4ncc[nH]4)cc3F)nc3ccc4[nH]ncc4c3c21]

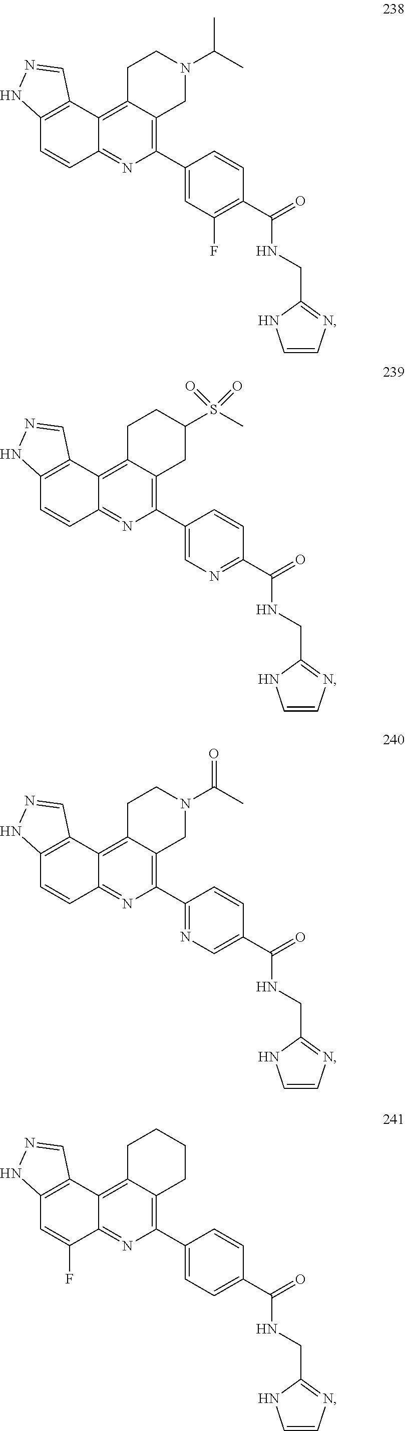 [SMILES: CC(C)N1CCc2c(c(-c3ccc(C(=O)NCc4ncc[nH]4)c(F)c3)nc3ccc4[nH]ncc4c23)C1 ; CS(=O)(=O)C1CCc2c(c(-c3ccc(C(=O)NCc4ncc[nH]4)nc3)nc3ccc4[nH]ncc4c23)C1 ; CC(=O)N1CCc2c(c(-c3ccc(C(=O)NCc4ncc[nH]4)cn3)nc3ccc4[nH]ncc4c23)C1 ; O=C(NCc1ncc[nH]1)c1ccc(-c2nc3c(F)cc4[nH]ncc4c3c3c2CCCC3)cc1]

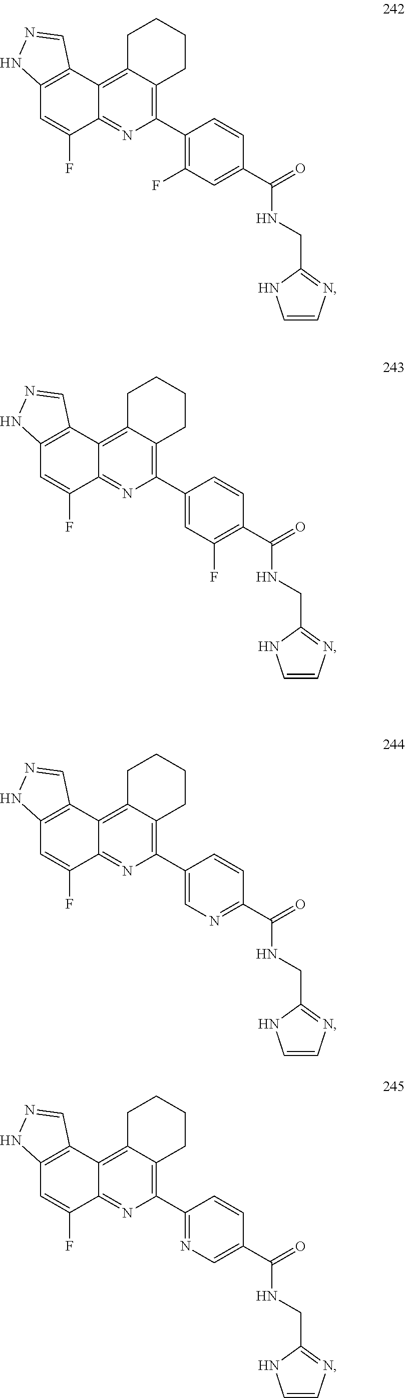 [SMILES: O=C(NCc1ncc[nH]1)c1ccc(-c2nc3c(F)cc4[nH]ncc4c3c3c2CCCC3)c(F)c1 ; O=C(NCc1ncc[nH]1)c1ccc(-c2nc3c(F)cc4[nH]ncc4c3c3c2CCCC3)cc1F ; O=C(NCc1ncc[nH]1)c1ccc(-c2nc3c(F)cc4[nH]ncc4c3c3c2CCCC3)cn1 ; O=C(NCc1ncc[nH]1)c1ccc(-c2nc3c(F)cc4[nH]ncc4c3c3c2CCCC3)nc1]

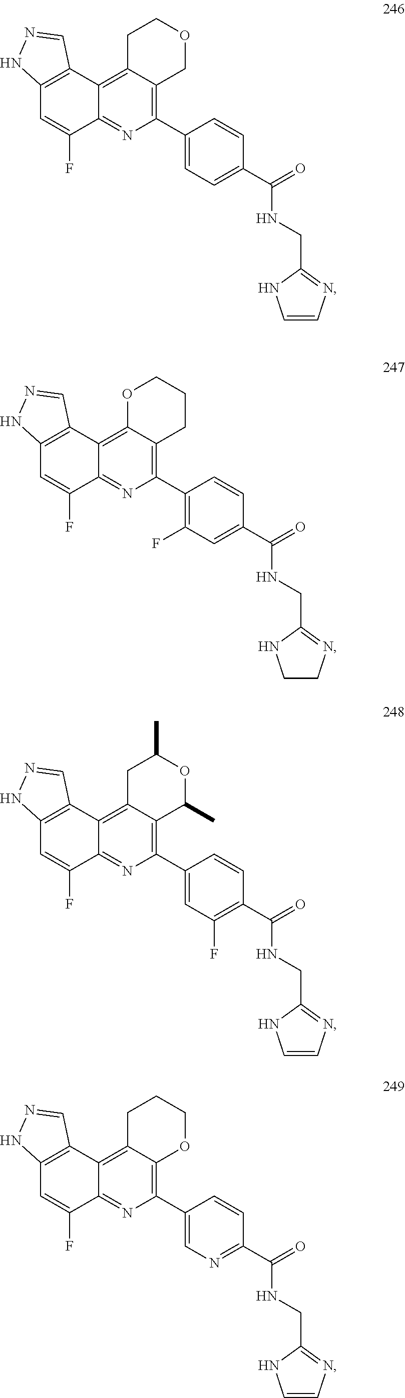 [SMILES: O=C(NCc1ncc[nH]1)c1ccc(-c2nc3c(F)cc4[nH]ncc4c3c3c2COCC3)cc1 ; O=C(NCC1=NCCN1)c1ccc(-c2nc3c(F)cc4[nH]ncc4c3c3c2CCCO3)c(F)c1 ; C[C@@H]1Cc2c(c(-c3ccc(C(=O)NCc4ncc[nH]4)c(F)c3)nc3c(F)cc4[nH]ncc4c23)[C@H](C)O1 ; O=C(NCc1ncc[nH]1)c1ccc(-c2nc3c(F)cc4[nH]ncc4c3c3c2OCCC3)cn1]

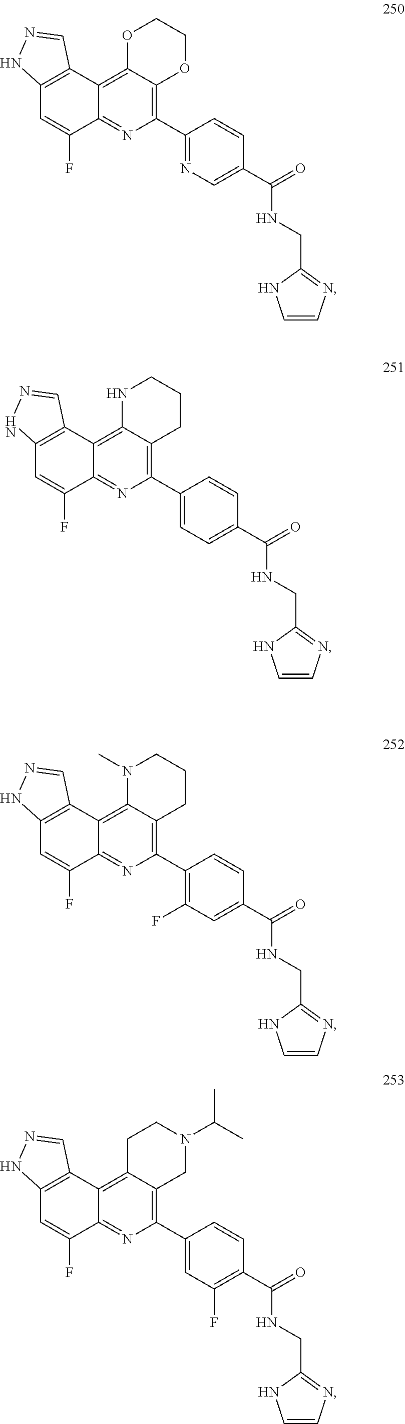 [SMILES: O=C(NCc1ncc[nH]1)c1ccc(-c2nc3c(F)cc4[nH]ncc4c3c3c2OCCO3)nc1 ; O=C(NCc1ncc[nH]1)c1ccc(-c2nc3c(F)cc4[nH]ncc4c3c3c2CCCN3)cc1 ; CN1CCCc2c(-c3ccc(C(=O)NCc4ncc[nH]4)cc3F)nc3c(F)cc4[nH]ncc4c3c21 ; CC(C)N1CCc2c(c(-c3ccc(C(=O)NCc4ncc[nH]4)c(F)c3)nc3c(F)cc4[nH]ncc4c23)C1]

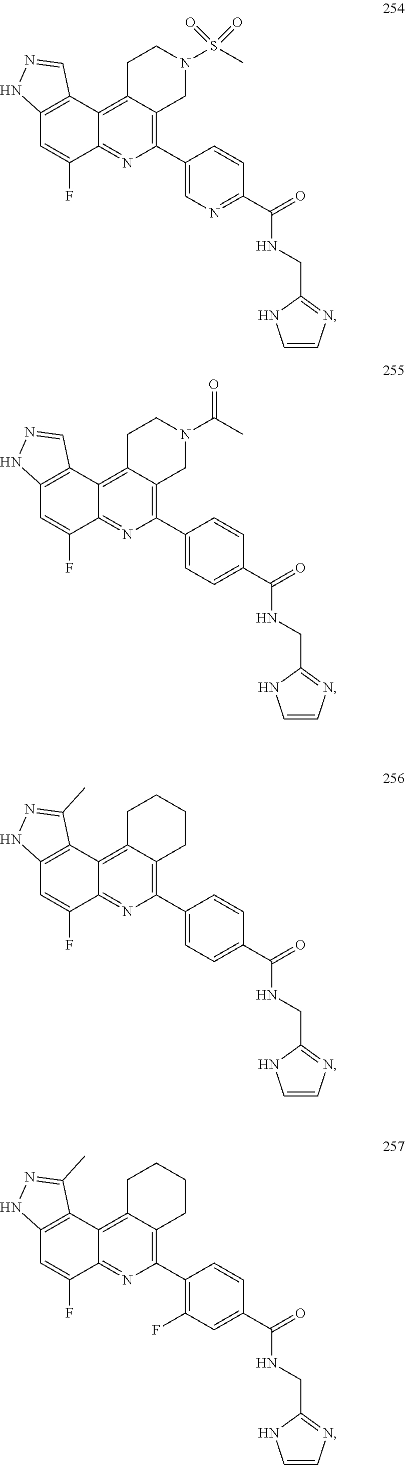 [SMILES: CS(=O)(=O)N1CCc2c(c(-c3ccc(C(=O)NCc4ncc[nH]4)nc3)nc3c(F)cc4[nH]ncc4c23)C1 ; CC(=O)N1CCc2c(c(-c3ccc(C(=O)NCc4ncc[nH]4)cc3)nc3c(F)cc4[nH]ncc4c23)C1 ; Cc1n[nH]c2cc(F)c3nc(-c4ccc(C(=O)NCc5ncc[nH]5)cc4)c4c(c3c12)CCCC4 ; Cc1n[nH]c2cc(F)c3nc(-c4ccc(C(=O)NCc5ncc[nH]5)cc4F)c4c(c3c12)CCCC4]

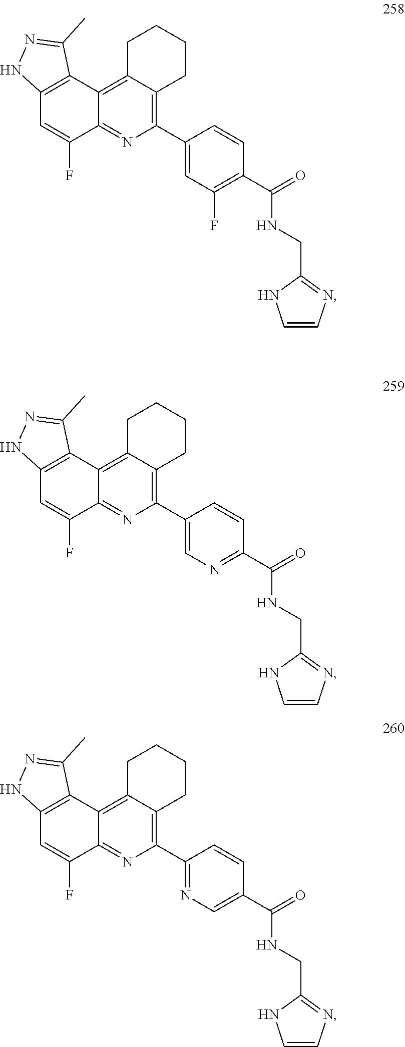 [SMILES: Cc1n[nH]c2cc(F)c3nc(-c4ccc(C(=O)NCc5ncc[nH]5)c(F)c4)c4c(c3c12)CCCC4 ; Cc1n[nH]c2cc(F)c3nc(-c4ccc(C(=O)NCc5ncc[nH]5)nc4)c4c(c3c12)CCCC4 ; Cc1n[nH]c2cc(F)c3nc(-c4ccc(C(=O)NCc5ncc[nH]5)cn4)c4c(c3c12)CCCC4]

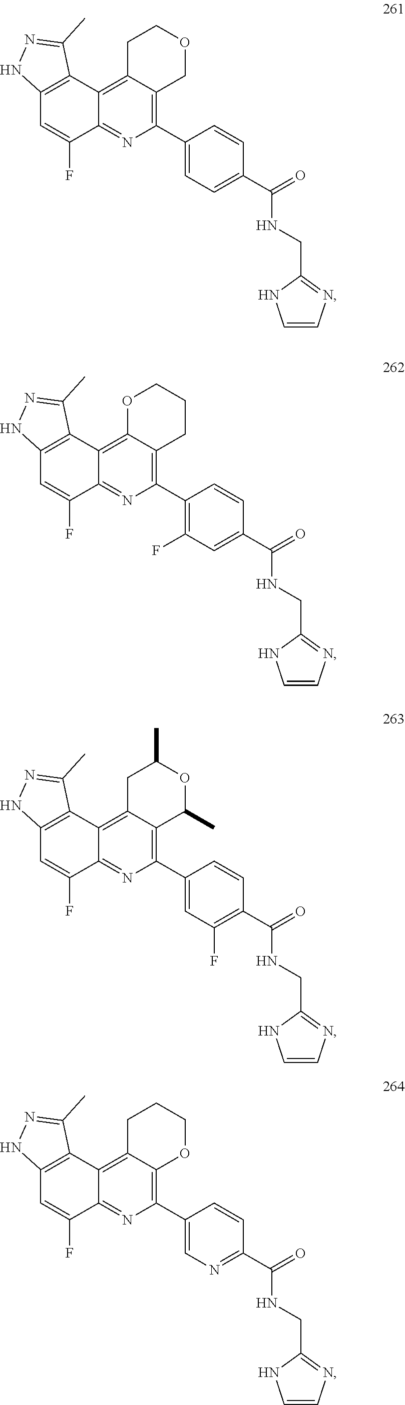 [SMILES: Cc1n[nH]c2cc(F)c3nc(-c4ccc(C(=O)NCc5ncc[nH]5)cc4)c4c(c3c12)CCOC4 ; Cc1n[nH]c2cc(F)c3nc(-c4ccc(C(=O)NCc5ncc[nH]5)cc4F)c4c(c3c12)OCCC4 ; Cc1n[nH]c2cc(F)c3nc(-c4ccc(C(=O)NCc5ncc[nH]5)c(F)c4)c4c(c3c12)C[C@@H](C)O[C@H]4C ; Cc1n[nH]c2cc(F)c3nc(-c4ccc(C(=O)NCc5ncc[nH]5)nc4)c4c(c3c12)CCCO4]

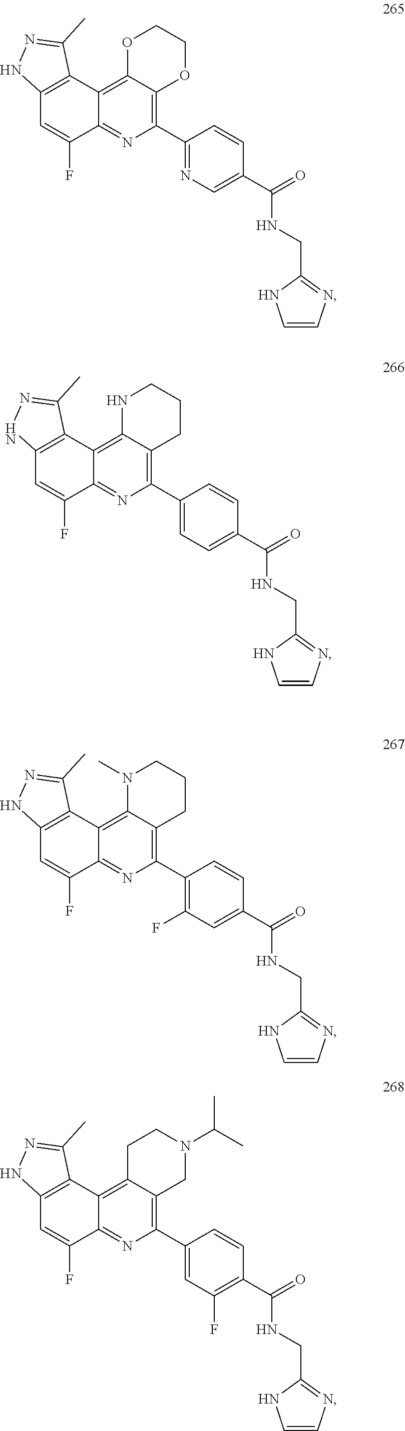 [SMILES: Cc1n[nH]c2cc(F)c3nc(-c4ccc(C(=O)NCc5ncc[nH]5)cn4)c4c(c3c12)OCCO4 ; Cc1n[nH]c2cc(F)c3nc(-c4ccc(C(=O)NCc5ncc[nH]5)cc4)c4c(c3c12)NCCC4 ; Cc1n[nH]c2cc(F)c3nc(-c4ccc(C(=O)NCc5ncc[nH]5)cc4F)c4c(c3c12)N(C)CCC4 ; Cc1n[nH]c2cc(F)c3nc(-c4ccc(C(=O)NCc5ncc[nH]5)c(F)c4)c4c(c3c12)CCN(C(C)C)C4]

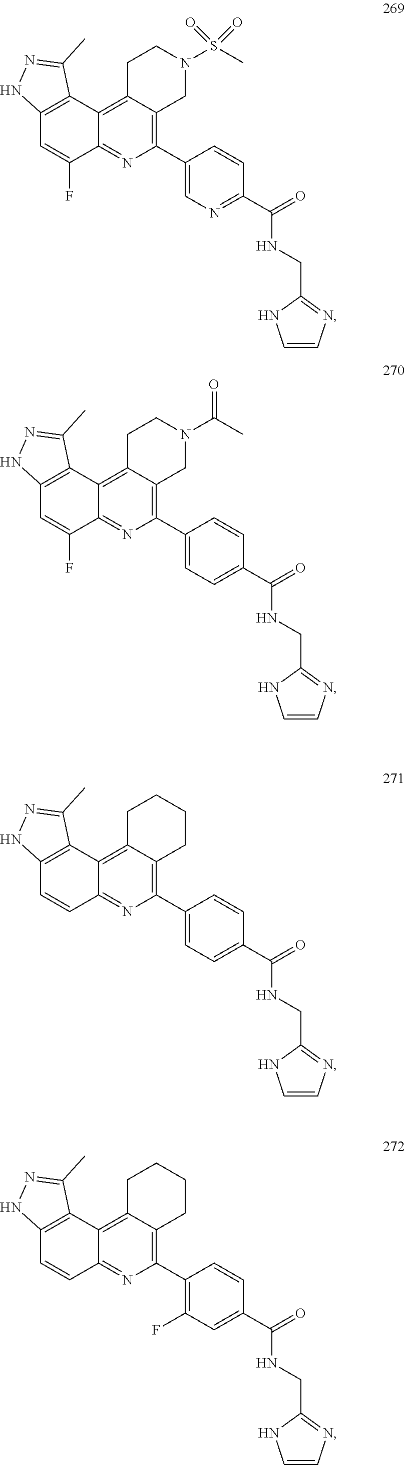 [SMILES: Cc1n[nH]c2cc(F)c3nc(-c4ccc(C(=O)NCc5ncc[nH]5)nc4)c4c(c3c12)CCN(S(C)(=O)=O)C4 ; CC(=O)N1CCc2c(c(-c3ccc(C(=O)NCc4ncc[nH]4)cc3)nc3c(F)cc4[nH]nc(C)c4c23)C1 ; Cc1n[nH]c2ccc3nc(-c4ccc(C(=O)NCc5ncc[nH]5)cc4)c4c(c3c12)CCCC4 ; Cc1n[nH]c2ccc3nc(-c4ccc(C(=O)NCc5ncc[nH]5)cc4F)c4c(c3c12)CCCC4]

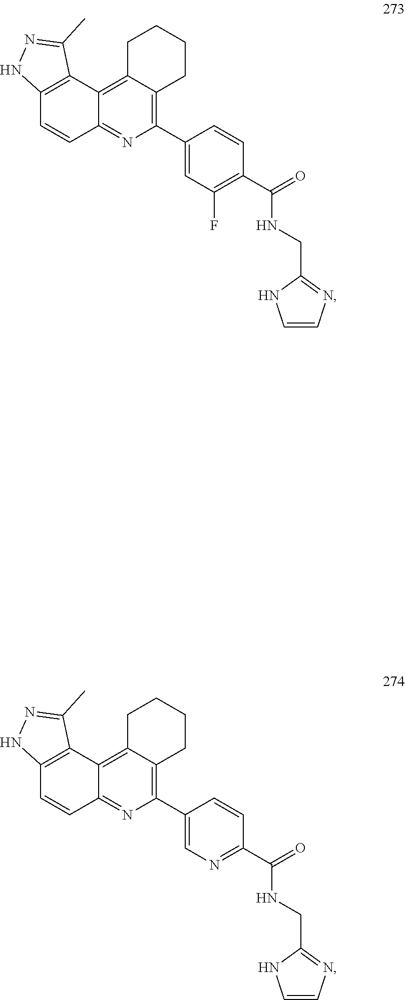 [SMILES: Cc1n[nH]c2ccc3nc(-c4ccc(C(=O)NCc5ncc[nH]5)c(F)c4)c4c(c3c12)CCCC4 ; Cc1n[nH]c2ccc3nc(-c4ccc(C(=O)NCc5ncc[nH]5)nc4)c4c(c3c12)CCCC4]

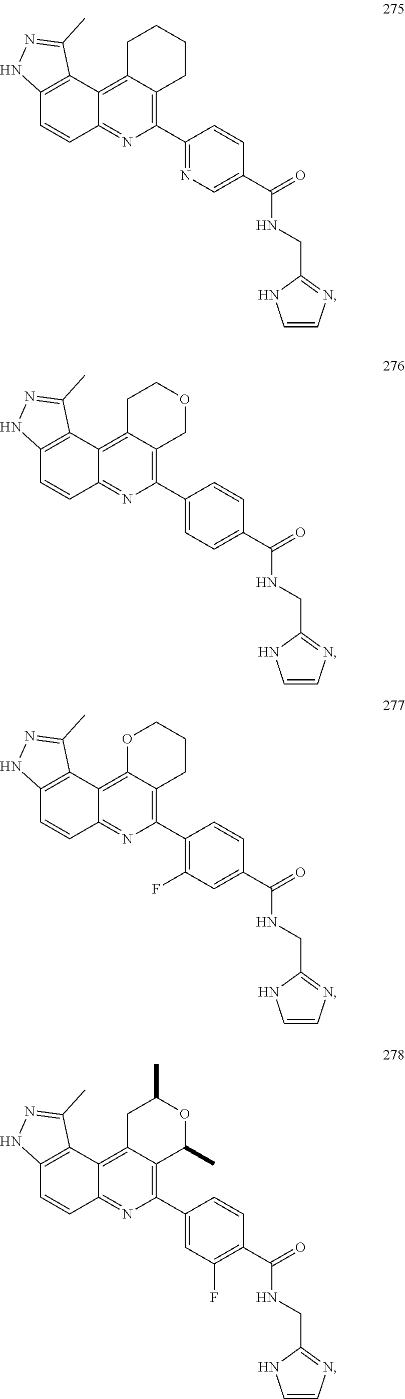 [SMILES: Cc1n[nH]c2ccc3nc(-c4ccc(C(=O)NCc5ncc[nH]5)cn4)c4c(c3c12)CCCC4 ; Cc1n[nH]c2ccc3nc(-c4ccc(C(=O)NCc5ncc[nH]5)cc4)c4c(c3c12)CCOC4 ; Cc1n[nH]c2ccc3nc(-c4ccc(C(=O)NCc5ncc[nH]5)cc4F)c4c(c3c12)OCCC4 ; Cc1n[nH]c2ccc3nc(-c4ccc(C(=O)NCc5ncc[nH]5)c(F)c4)c4c(c3c12)C[C@@H](C)O[C@H]4C]

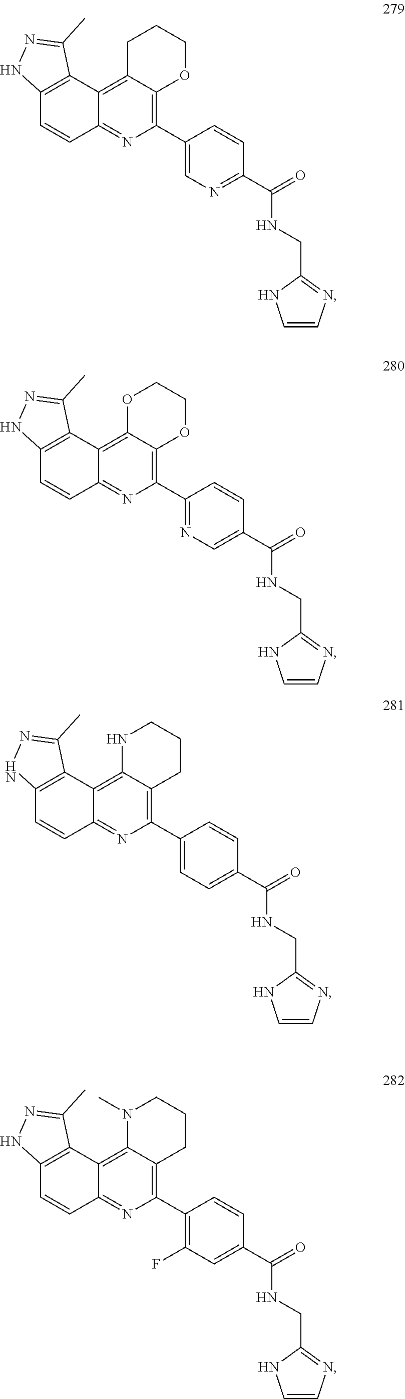 [SMILES: Cc1n[nH]c2ccc3nc(-c4ccc(C(=O)NCc5ncc[nH]5)nc4)c4c(c3c12)CCCO4 ; Cc1n[nH]c2ccc3nc(-c4ccc(C(=O)NCc5ncc[nH]5)cn4)c4c(c3c12)OCCO4 ; Cc1n[nH]c2ccc3nc(-c4ccc(C(=O)NCc5ncc[nH]5)cc4)c4c(c3c12)NCCC4 ; Cc1n[nH]c2ccc3nc(-c4ccc(C(=O)NCc5ncc[nH]5)cc4F)c4c(c3c12)N(C)CCC4]

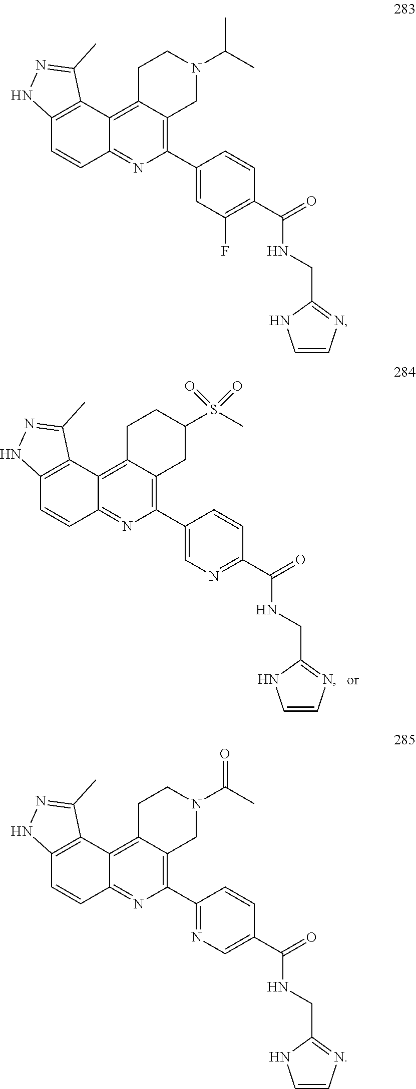 [SMILES: Cc1n[nH]c2ccc3nc(-c4ccc(C(=O)NCc5ncc[nH]5)c(F)c4)c4c(c3c12)CCN(C(C)C)C4 ; Cc1n[nH]c2ccc3nc(-c4ccc(C(=O)NCc5ncc[nH]5)nc4)c4c(c3c12)CCC(S(C)(=O)=O)C4 ; "or" ; CC(=O)N1CCc2c(c(-c3ccc(C(=O)NCc4ncc[nH]4)cn3)nc3ccc4[nH]nc(C)c4c23)C1]

In certain embodiments, the compound has a structure of, or is a pharmaceutically acceptable salt of, one of the following:

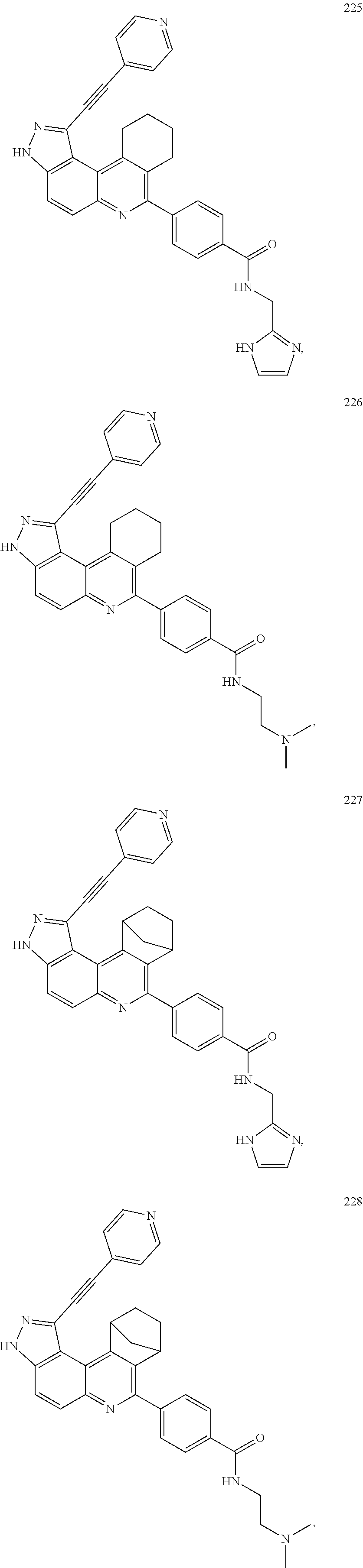 [SMILES: O=C(NCc1ncc[nH]1)c1ccc(-c2nc3ccc4[nH]nc(C#Cc5ccncc5)c4c3c3c2CCCC3)cc1 ; CN(C)CCNC(=O)c1ccc(-c2nc3ccc4[nH]nc(C#Cc5ccncc5)c4c3c3c2CCCC3)cc1 ; O=C(NCc1ncc[nH]1)c1ccc(-c2nc3ccc4[nH]nc(C#Cc5ccncc5)c4c3c3c2C2CCC3C2)cc1 ; CN(C)CCNC(=O)c1ccc(-c2nc3ccc4[nH]nc(C#Cc5ccncc5)c4c3c3c2C2CCC3C2)cc1]

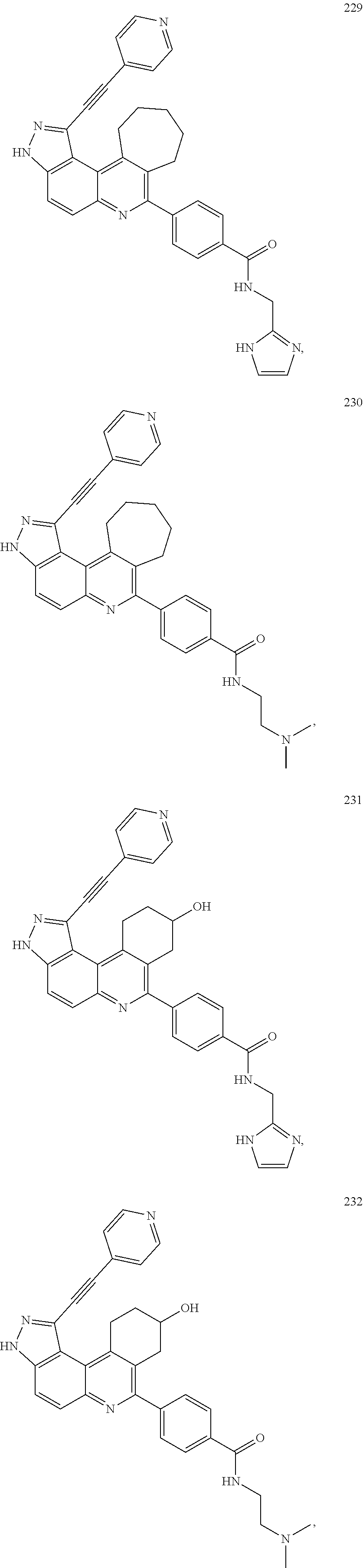 [SMILES: O=C(NCc1ncc[nH]1)c1ccc(-c2nc3ccc4[nH]nc(C#Cc5ccncc5)c4c3c3c2CCCCC3)cc1 ; CN(C)CCNC(=O)c1ccc(-c2nc3ccc4[nH]nc(C#Cc5ccncc5)c4c3c3c2CCCCC3)cc1 ; O=C(NCc1ncc[nH]1)c1ccc(-c2nc3ccc4[nH]nc(C#Cc5ccncc5)c4c3c3c2CC(O)CC3)cc1 ; CN(C)CCNC(=O)c1ccc(-c2nc3ccc4[nH]nc(C#Cc5ccncc5)c4c3c3c2CC(O)CC3)cc1]

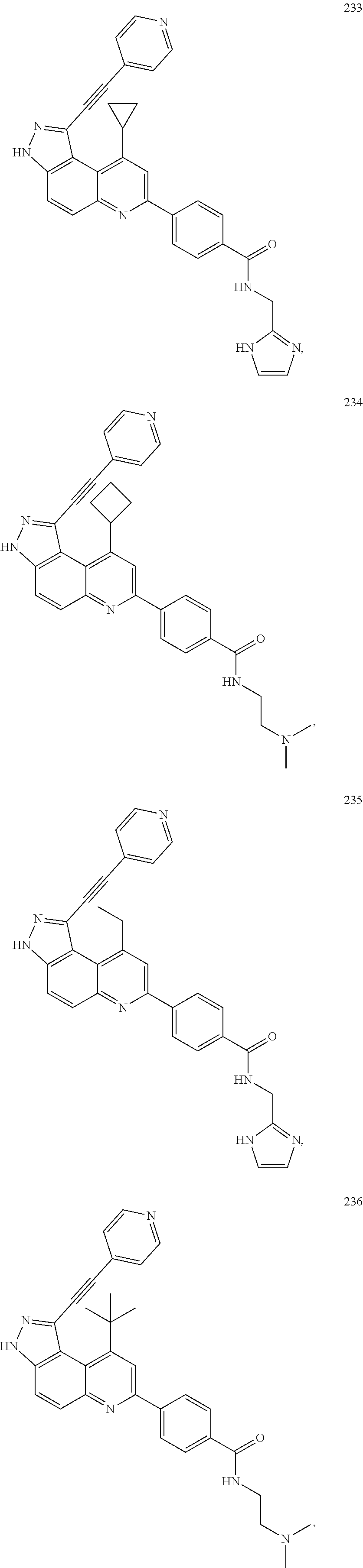 [SMILES: O=C(NCc1ncc[nH]1)c1ccc(-c2cc(C3CC3)c3c(ccc4[nH]nc(C#Cc5ccncc5)c43)n2)cc1 ; CN(C)CCNC(=O)c1ccc(-c2cc(C3CCC3)c3c(ccc4[nH]nc(C#Cc5ccncc5)c43)n2)cc1 ; CCc1cc(-c2ccc(C(=O)NCc3ncc[nH]3)cc2)nc2ccc3[nH]nc(C#Cc4ccncc4)c3c12 ; CN(C)CCNC(=O)c1ccc(-c2cc(C(C)(C)C)c3c(ccc4[nH]nc(C#Cc5ccncc5)c43)n2)cc1]

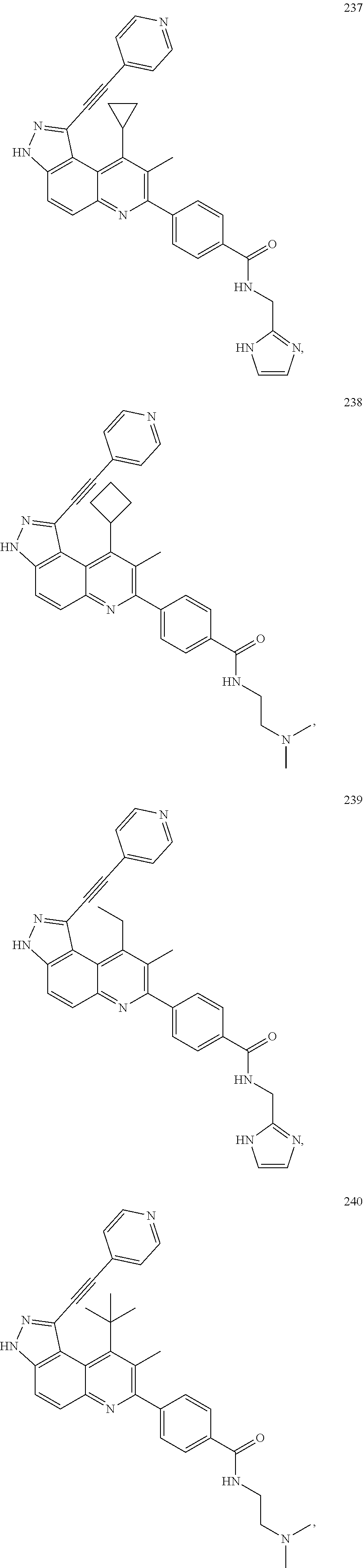 [SMILES: Cc1c(-c2ccc(C(=O)NCc3ncc[nH]3)cc2)nc2ccc3[nH]nc(C#Cc4ccncc4)c3c2c1C1CC1 ; Cc1c(-c2ccc(C(=O)NCCN(C)C)cc2)nc2ccc3[nH]nc(C#Cc4ccncc4)c3c2c1C1CCC1 ; CCc1c(C)c(-c2ccc(C(=O)NCc3ncc[nH]3)cc2)nc2ccc3[nH]nc(C#Cc4ccncc4)c3c12 ; Cc1c(-c2ccc(C(=O)NCCN(C)C)cc2)nc2ccc3[nH]nc(C#Cc4ccncc4)c3c2c1C(C)(C)C]

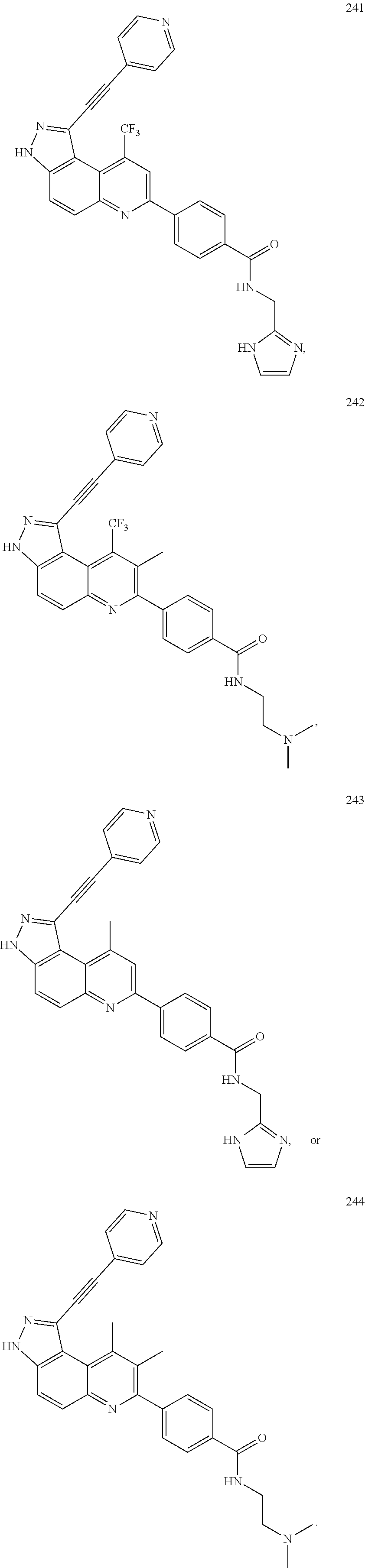 [SMILES: O=C(NCc1ncc[nH]1)c1ccc(-c2cc(C(F)(F)F)c3c(ccc4[nH]nc(C#Cc5ccncc5)c43)n2)cc1 ; Cc1c(-c2ccc(C(=O)NCCN(C)C)cc2)nc2ccc3[nH]nc(C#Cc4ccncc4)c3c2c1C(F)(F)F ; Cc1cc(-c2ccc(C(=O)NCc3ncc[nH]3)cc2)nc2ccc3[nH]nc(C#Cc4ccncc4)c3c12 ; Cc1c(-c2ccc(C(=O)NCCN(C)C)cc2)nc2ccc3[nH]nc(C#Cc4ccncc4)c3c2c1C]

In certain embodiments, the compound has a structure of, or is a pharmaceutically acceptable salt of, one of the following:

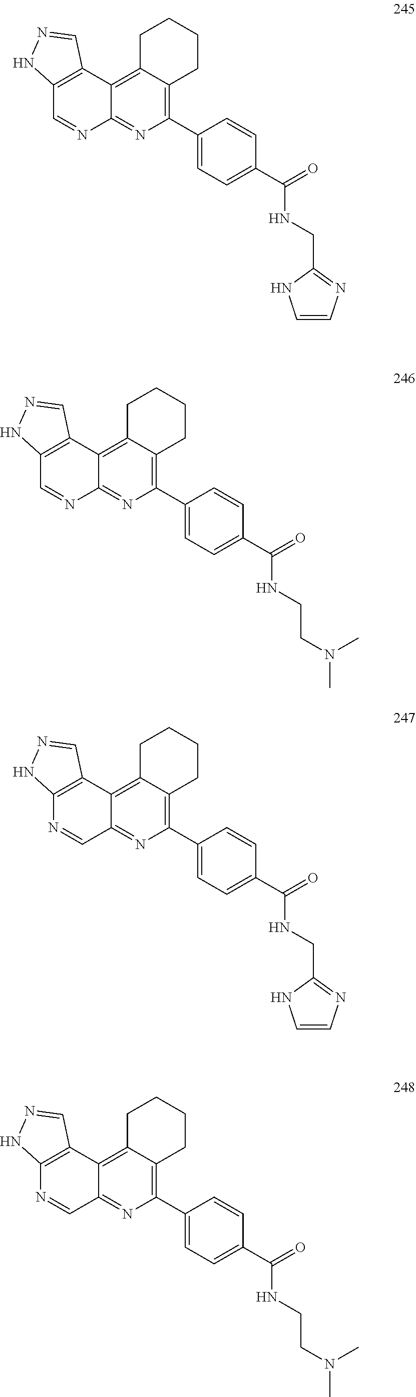 [SMILES: O=C(NCc1ncc[nH]1)c1ccc(-c2nc3ncc4[nH]ncc4c3c3c2CCCC3)cc1 ; CN(C)CCNC(=O)c1ccc(-c2nc3ncc4[nH]ncc4c3c3c2CCCC3)cc1 ; O=C(NCc1ncc[nH]1)c1ccc(-c2nc3cnc4[nH]ncc4c3c3c2CCCC3)cc1 ; CN(C)CCNC(=O)c1ccc(-c2nc3cnc4[nH]ncc4c3c3c2CCCC3)cc1]

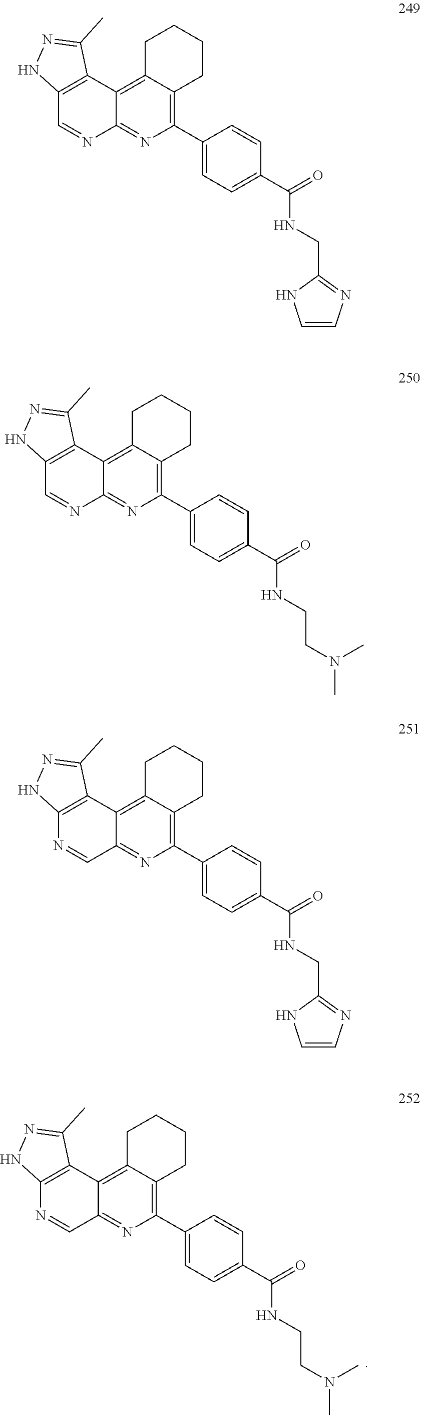 [SMILES: Cc1n[nH]c2cnc3nc(-c4ccc(C(=O)NCc5ncc[nH]5)cc4)c4c(c3c12)CCCC4 ; Cc1n[nH]c2cnc3nc(-c4ccc(C(=O)NCCN(C)C)cc4)c4c(c3c12)CCCC4 ; Cc1n[nH]c2ncc3nc(-c4ccc(C(=O)NCc5ncc[nH]5)cc4)c4c(c3c12)CCCC4 ; Cc1n[nH]c2ncc3nc(-c4ccc(C(=O)NCCN(C)C)cc4)c4c(c3c12)CCCC4]

In certain embodiments, the compound has a structure of, or is a pharmaceutically acceptable salt of, one of the following:

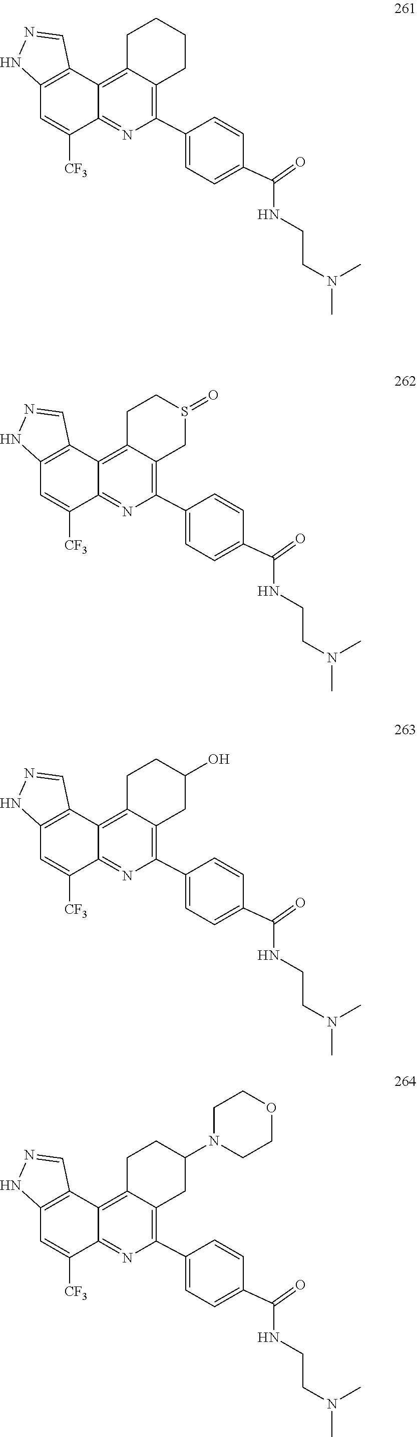 [SMILES: CN(C)CCNC(=O)c1ccc(-c2nc3c(C(F)(F)F)cc4[nH]ncc4c3c3c2CCCC3)cc1 ; CN(C)CCNC(=O)c1ccc(-c2nc3c(C(F)(F)F)cc4[nH]ncc4c3c3c2CS(=O)CC3)cc1 ; CN(C)CCNC(=O)c1ccc(-c2nc3c(C(F)(F)F)cc4[nH]ncc4c3c3c2CC(O)CC3)cc1 ; CN(C)CCNC(=O)c1ccc(-c2nc3c(C(F)(F)F)cc4[nH]ncc4c3c3c2CC(N2CCOCC2)CC3)cc1]

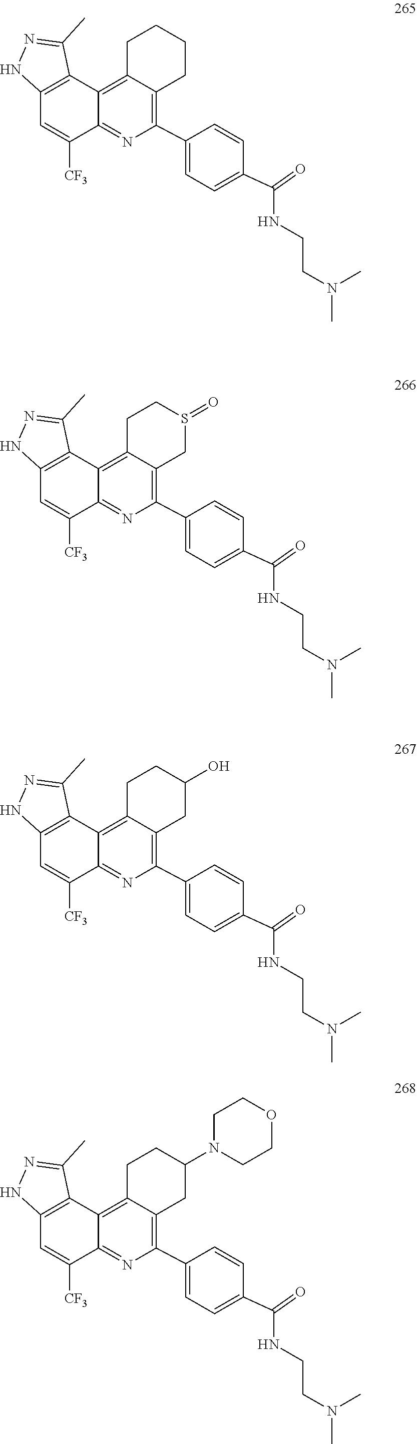 [SMILES: Cc1n[nH]c2cc(C(F)(F)F)c3nc(-c4ccc(C(=O)NCCN(C)C)cc4)c4c(c3c12)CCCC4 ; Cc1n[nH]c2cc(C(F)(F)F)c3nc(-c4ccc(C(=O)NCCN(C)C)cc4)c4c(c3c12)CCS(=O)C4 ; Cc1n[nH]c2cc(C(F)(F)F)c3nc(-c4ccc(C(=O)NCCN(C)C)cc4)c4c(c3c12)CCC(O)C4 ; Cc1n[nH]c2cc(C(F)(F)F)c3nc(-c4ccc(C(=O)NCCN(C)C)cc4)c4c(c3c12)CCC(N1CCOCC1)C4]

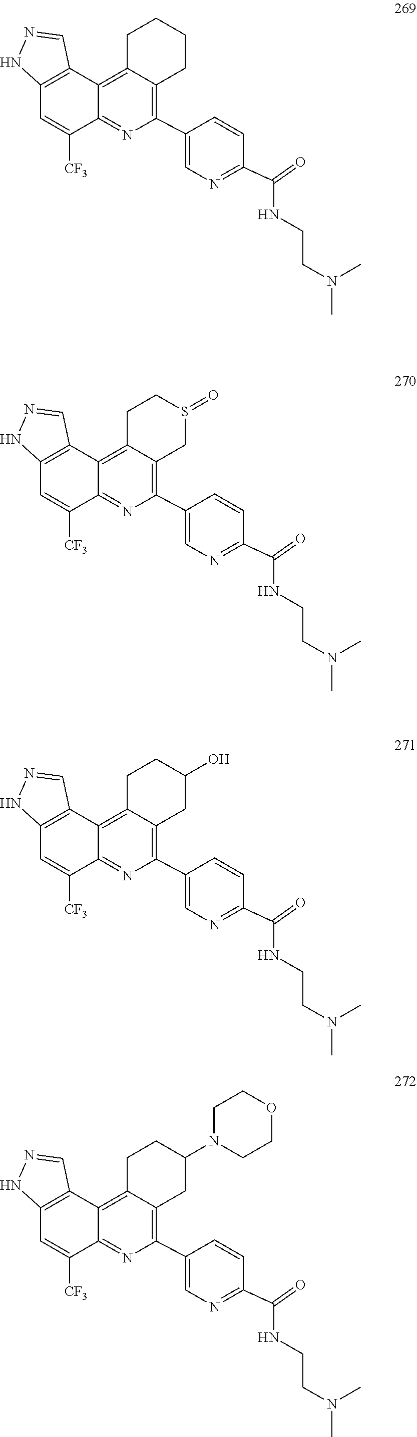 [SMILES: CN(C)CCNC(=O)c1ccc(-c2nc3c(C(F)(F)F)cc4[nH]ncc4c3c3c2CCCC3)cn1 ; CN(C)CCNC(=O)c1ccc(-c2nc3c(C(F)(F)F)cc4[nH]ncc4c3c3c2CS(=O)CC3)cn1 ; CN(C)CCNC(=O)c1ccc(-c2nc3c(C(F)(F)F)cc4[nH]ncc4c3c3c2CC(O)CC3)cn1 ; CN(C)CCNC(=O)c1ccc(-c2nc3c(C(F)(F)F)cc4[nH]ncc4c3c3c2CC(N2CCOCC2)CC3)cn1]

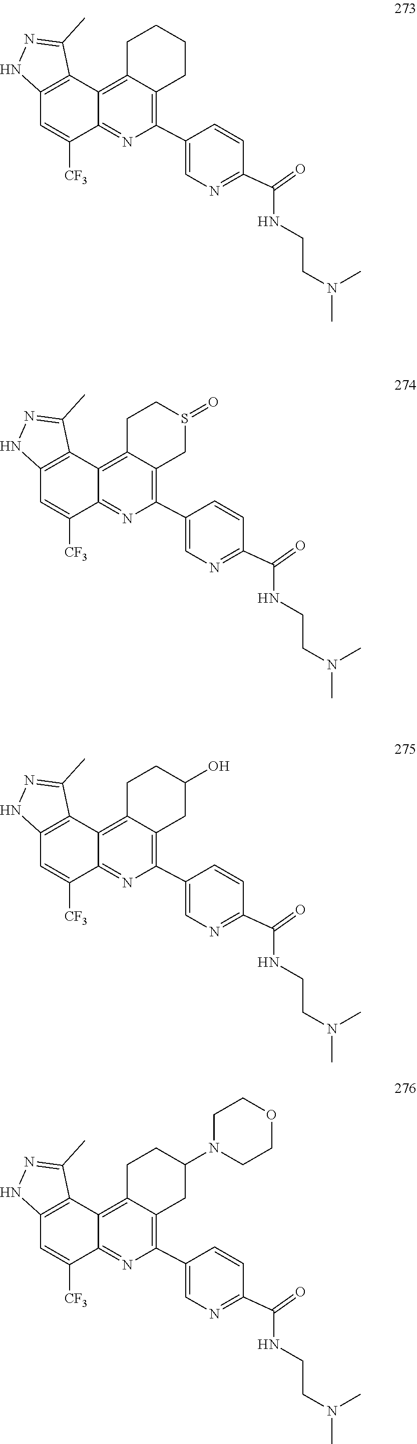 [SMILES: Cc1n[nH]c2cc(C(F)(F)F)c3nc(-c4ccc(C(=O)NCCN(C)C)nc4)c4c(c3c12)CCCC4 ; Cc1n[nH]c2cc(C(F)(F)F)c3nc(-c4ccc(C(=O)NCCN(C)C)nc4)c4c(c3c12)CCS(=O)C4 ; Cc1n[nH]c2cc(C(F)(F)F)c3nc(-c4ccc(C(=O)NCCN(C)C)nc4)c4c(c3c12)CCC(O)C4 ; Cc1n[nH]c2cc(C(F)(F)F)c3nc(-c4ccc(C(=O)NCCN(C)C)nc4)c4c(c3c12)CCC(N1CCOCC1)C4]

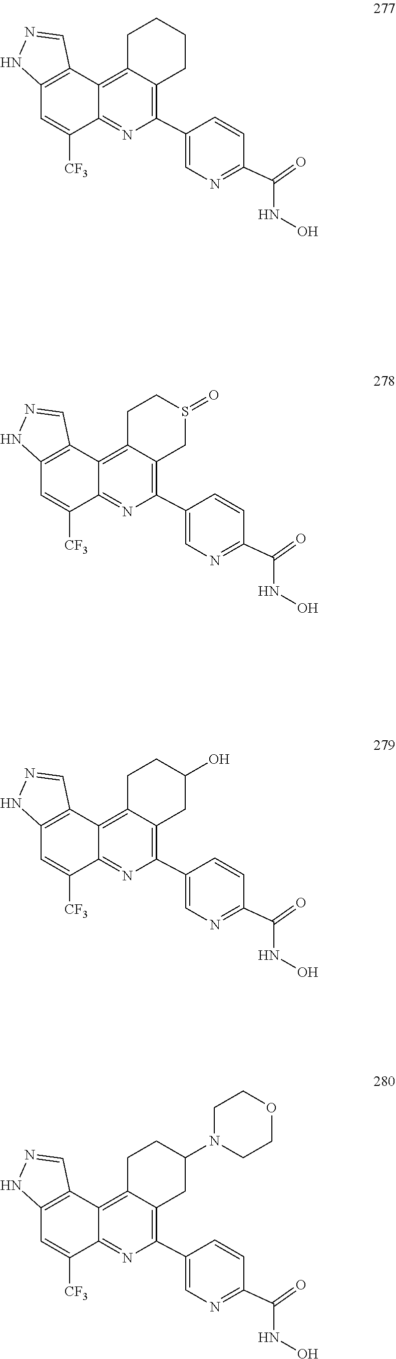 [SMILES: O=C(NO)c1ccc(-c2nc3c(C(F)(F)F)cc4[nH]ncc4c3c3c2CCCC3)cn1 ; O=C(NO)c1ccc(-c2nc3c(C(F)(F)F)cc4[nH]ncc4c3c3c2CS(=O)CC3)cn1 ; O=C(NO)c1ccc(-c2nc3c(C(F)(F)F)cc4[nH]ncc4c3c3c2CC(O)CC3)cn1 ; O=C(NO)c1ccc(-c2nc3c(C(F)(F)F)cc4[nH]ncc4c3c3c2CC(N2CCOCC2)CC3)cn1]

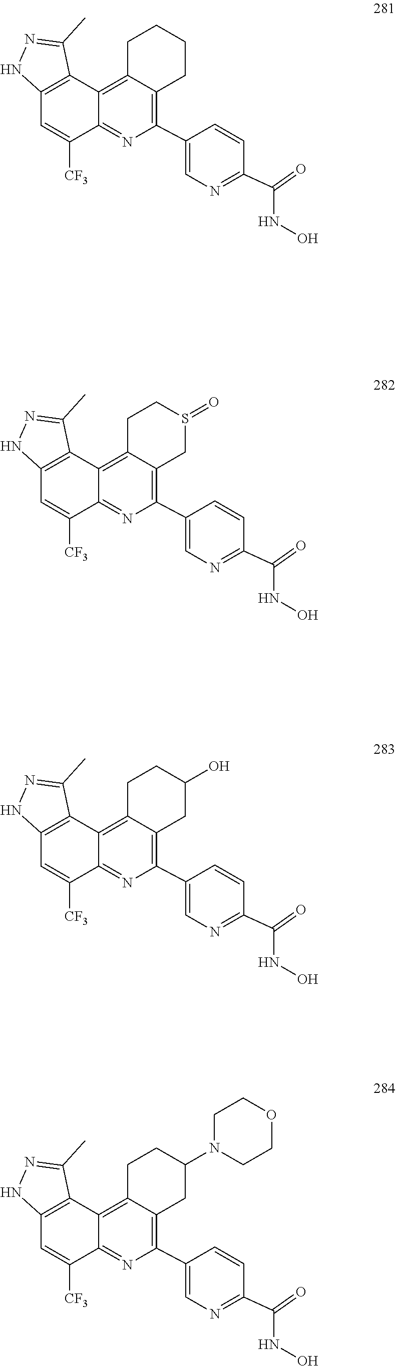 [SMILES: Cc1n[nH]c2cc(C(F)(F)F)c3nc(-c4ccc(C(=O)NO)nc4)c4c(c3c12)CCCC4 ; Cc1n[nH]c2cc(C(F)(F)F)c3nc(-c4ccc(C(=O)NO)nc4)c4c(c3c12)CCS(=O)C4 ; Cc1n[nH]c2cc(C(F)(F)F)c3nc(-c4ccc(C(=O)NO)nc4)c4c(c3c12)CCC(O)C4 ; Cc1n[nH]c2cc(C(F)(F)F)c3nc(-c4ccc(C(=O)NO)nc4)c4c(c3c12)CCC(N1CCOCC1)C4]

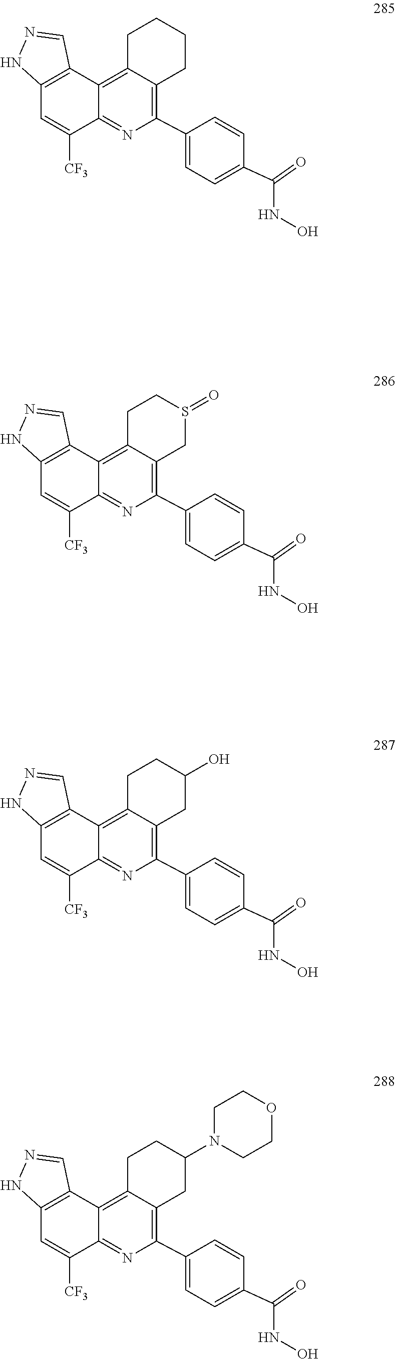 [SMILES: O=C(NO)c1ccc(-c2nc3c(C(F)(F)F)cc4[nH]ncc4c3c3c2CCCC3)cc1 ; O=C(NO)c1ccc(-c2nc3c(C(F)(F)F)cc4[nH]ncc4c3c3c2CS(=O)CC3)cc1 ; O=C(NO)c1ccc(-c2nc3c(C(F)(F)F)cc4[nH]ncc4c3c3c2CC(O)CC3)cc1 ; O=C(NO)c1ccc(-c2nc3c(C(F)(F)F)cc4[nH]ncc4c3c3c2CC(N2CCOCC2)CC3)cc1]

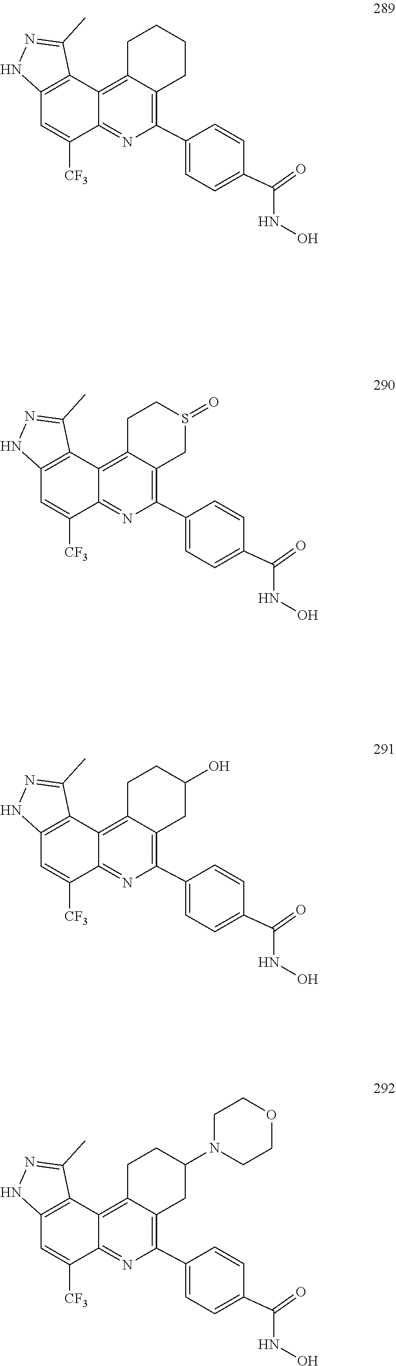 [SMILES: Cc1n[nH]c2cc(C(F)(F)F)c3nc(-c4ccc(C(=O)NO)cc4)c4c(c3c12)CCCC4 ; Cc1n[nH]c2cc(C(F)(F)F)c3nc(-c4ccc(C(=O)NO)cc4)c4c(c3c12)CCS(=O)C4 ; Cc1n[nH]c2cc(C(F)(F)F)c3nc(-c4ccc(C(=O)NO)cc4)c4c(c3c12)CCC(O)C4 ; Cc1n[nH]c2cc(C(F)(F)F)c3nc(-c4ccc(C(=O)NO)cc4)c4c(c3c12)CCC(N1CCOCC1)C4]

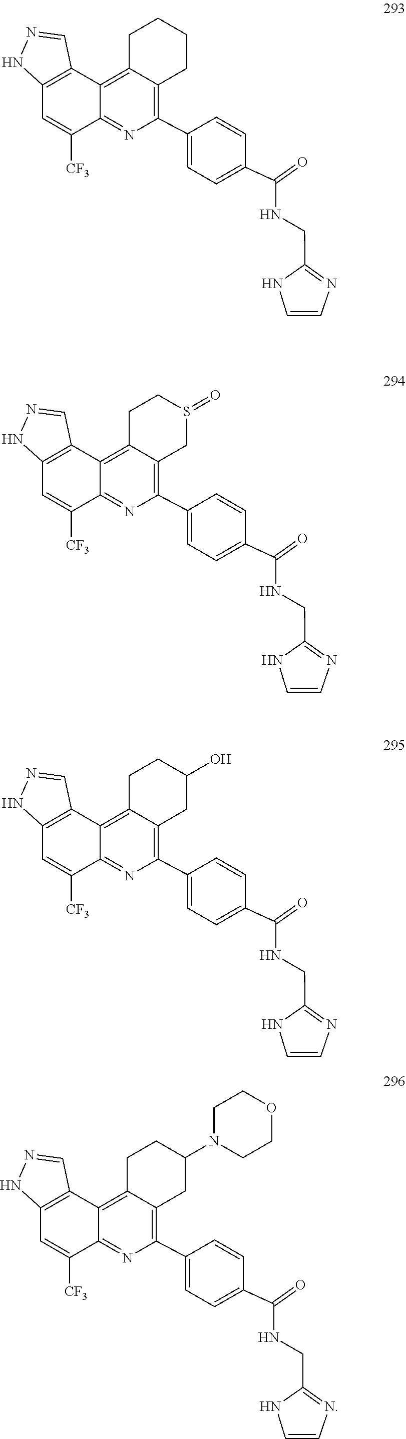 [SMILES: O=C(NCc1ncc[nH]1)c1ccc(-c2nc3c(C(F)(F)F)cc4[nH]ncc4c3c3c2CCCC3)cc1 ; O=C(NCc1ncc[nH]1)c1ccc(-c2nc3c(C(F)(F)F)cc4[nH]ncc4c3c3c2CS(=O)CC3)cc1 ; O=C(NCc1ncc[nH]1)c1ccc(-c2nc3c(C(F)(F)F)cc4[nH]ncc4c3c3c2CC(O)CC3)cc1 ; O=C(NCc1ncc[nH]1)c1ccc(-c2nc3c(C(F)(F)F)cc4[nH]ncc4c3c3c2CC(N2CCOCC2)CC3)cc1]

In certain embodiments, the compound can have the structure of formula II:

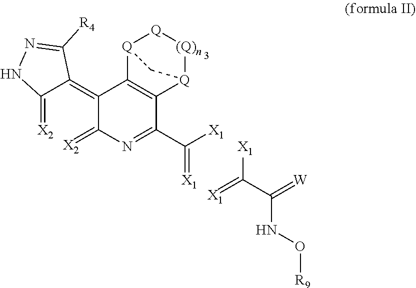

or a pharmaceutically acceptable salt thereof, wherein:
each X₁ is independently N, CH, or a C-halogen;
each X₂ is independently N, CH, a C-halogen, C—CHₙ₁Xₘ where n1+m=3 and X is a halogen, C—OCF₃, C—OCHF₂, C—OMe, C—Me, C—Et, C-tBu, C-iPr, C—N-alkyl, C—N-heteroalkyl, C—O-alkyl, or C—O-heteroalkyl;
each Q is independently O, NH, substituted N, CH₂, or substituted C, S, SO, or SO₂, and each Q, when taken together, form a 6-, 7-, or 8-membered ring or bicyclic ring;
n3 is 1, 2, or 3;
R₄ is an alkyl, cycloalkyl, heteroalkyl, or heterocycloalkyl;
W is O, NH, or NMe; and
R₉ is an alkyl or a heteroalkyl, with the proviso that R₉ is not H.

The compound can have a structure of:

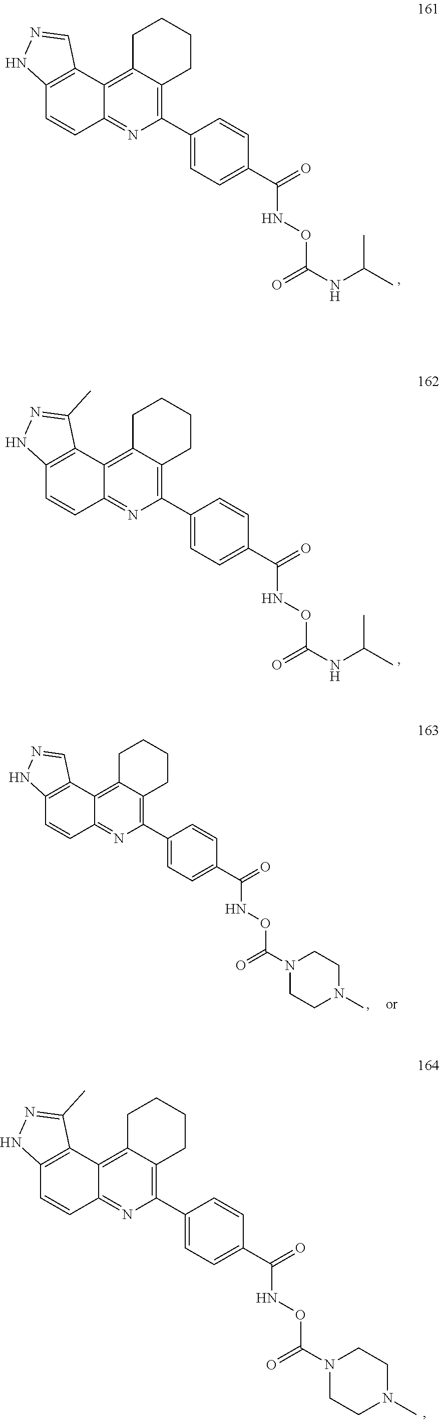 [SMILES: CC(C)NC(=O)ONC(=O)c1ccc(-c2nc3ccc4[nH]ncc4c3c3c2CCCC3)cc1 ; Cc1n[nH]c2ccc3nc(-c4ccc(C(=O)NOC(=O)NC(C)C)cc4)c4c(c3c12)CCCC4 ; CN1CCN(C(=O)ONC(=O)c2ccc(-c3nc4ccc5[nH]ncc5c4c4c3CCCC4)cc2)CC1 ; Cc1n[nH]c2ccc3nc(-c4ccc(C(=O)NOC(=O)N5CCN(C)CC5)cc4)c4c(c3c12)CCCC4]

or be a pharmaceutically acceptable salt of any of the foregoing structures.

In certain embodiments, the compound has a structure of, or is a pharmaceutically acceptable salt of, one of the following:

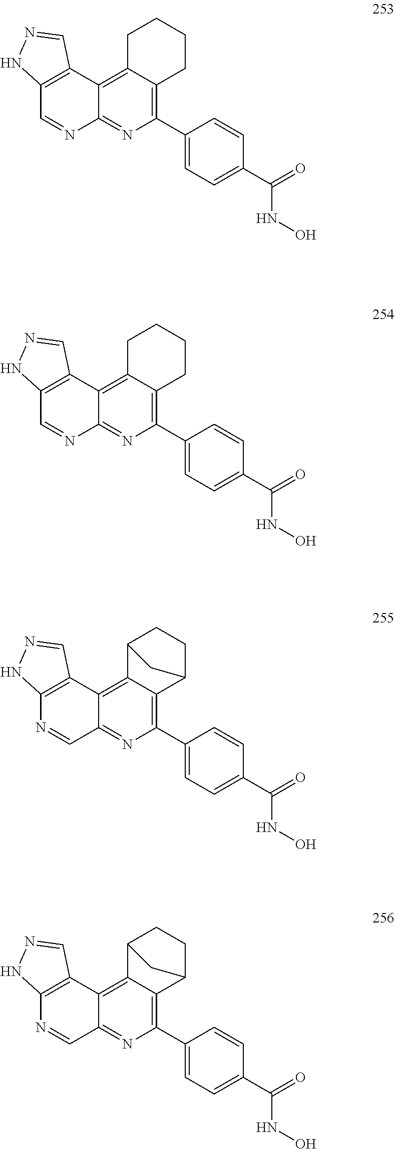 [SMILES: O=C(NO)c1ccc(-c2nc3ncc4[nH]ncc4c3c3c2CCCC3)cc1 ; O=C(NO)c1ccc(-c2nc3ncc4[nH]ncc4c3c3c2CCCC3)cc1 ; O=C(NO)c1ccc(-c2nc3cnc4[nH]ncc4c3c3c2C2CCC3C2)cc1 ; O=C(NO)c1ccc(-c2nc3cnc4[nH]ncc4c3c3c2C2CCC3C2)cc1]

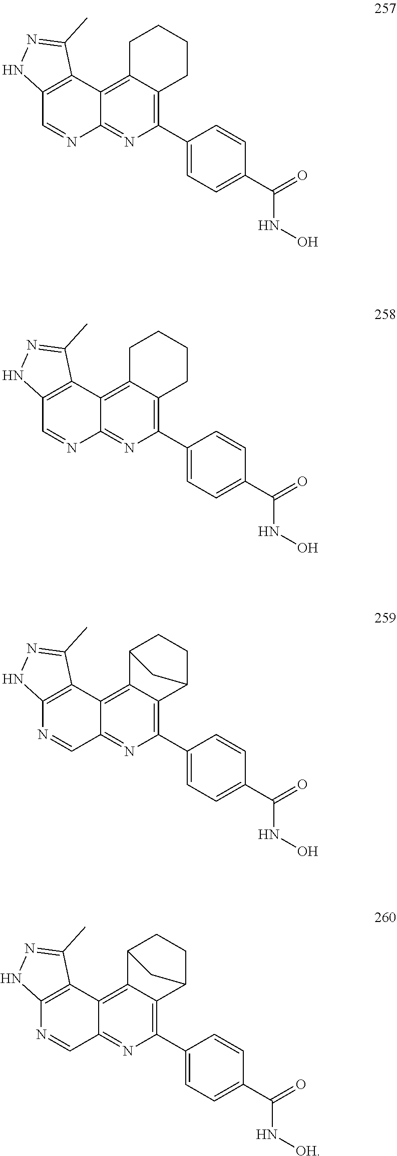 [SMILES: Cc1n[nH]c2cnc3nc(-c4ccc(C(=O)NO)cc4)c4c(c3c12)CCCC4 ; Cc1n[nH]c2cnc3nc(-c4ccc(C(=O)NO)cc4)c4c(c3c12)CCCC4 ; Cc1n[nH]c2ncc3nc(-c4ccc(C(=O)NO)cc4)c4c(c3c12)C1CCC4C1 ; Cc1n[nH]c2ncc3nc(-c4ccc(C(=O)NO)cc4)c4c(c3c12)C1CCC4C1]

In certain embodiments, the compound has a structure of, or is a pharmaceutically acceptable salt of, one of the following:

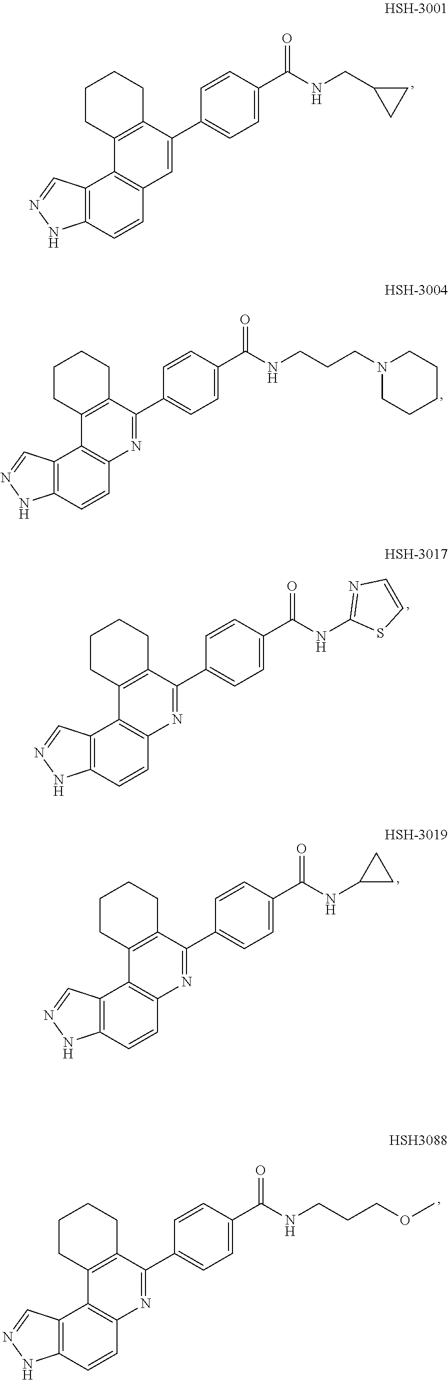 [SMILES: O=C(NCC1CC1)c1ccc(-c2cc3ccc4[nH]ncc4c3c3c2CCCC3)cc1 ; O=C(NCCCN1CCCCC1)c1ccc(-c2nc3ccc4[nH]ncc4c3c3c2CCCC3)cc1 ; O=C(Nc1nccs1)c1ccc(-c2nc3ccc4[nH]ncc4c3c3c2CCCC3)cc1 ; O=C(NC1CC1)c1ccc(-c2nc3ccc4[nH]ncc4c3c3c2CCCC3)cc1 ; COCCCNC(=O)c1ccc(-c2nc3ccc4[nH]ncc4c3c3c2CCCC3)cc1]

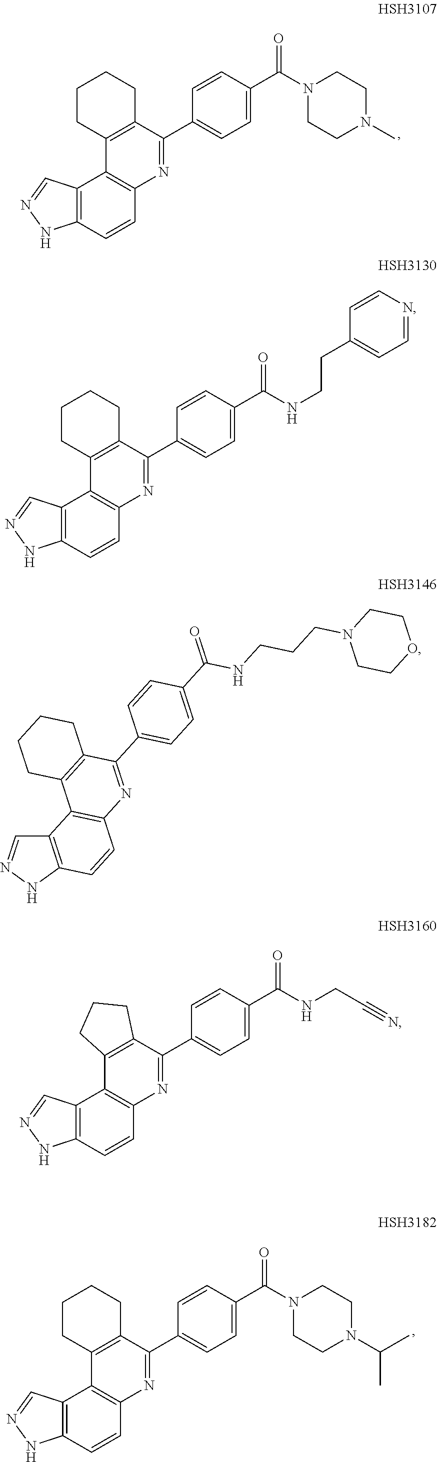 [SMILES: CN1CCN(C(=O)c2ccc(-c3nc4ccc5[nH]ncc5c4c4c3CCCC4)cc2)CC1 ; O=C(NCCc1ccncc1)c1ccc(-c2nc3ccc4[nH]ncc4c3c3c2CCCC3)cc1 ; O=C(NCCCN1CCOCC1)c1ccc(-c2nc3ccc4[nH]ncc4c3c3c2CCCC3)cc1 ; N#CCNC(=O)c1ccc(-c2nc3ccc4[nH]ncc4c3c3c2CCC3)cc1 ; CC(C)N1CCN(C(=O)c2ccc(-c3nc4ccc5[nH]ncc5c4c4c3CCCC4)cc2)CC1]

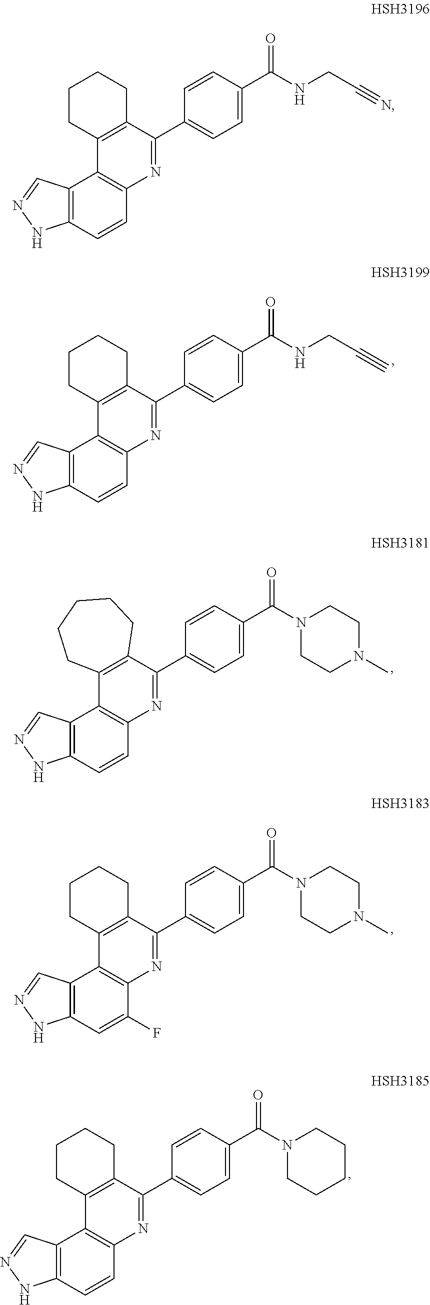 [SMILES: N#CCNC(=O)c1ccc(-c2nc3ccc4[nH]ncc4c3c3c2CCCC3)cc1 ; C#CCNC(=O)c1ccc(-c2nc3ccc4[nH]ncc4c3c3c2CCCC3)cc1 ; CN1CCN(C(=O)c2ccc(-c3nc4ccc5[nH]ncc5c4c4c3CCCCC4)cc2)CC1 ; CN1CCN(C(=O)c2ccc(-c3nc4c(F)cc5[nH]ncc5c4c4c3CCCC4)cc2)CC1 ; O=C(c1ccc(-c2nc3ccc4[nH]ncc4c3c3c2CCCC3)cc1)N1CCCCC1]

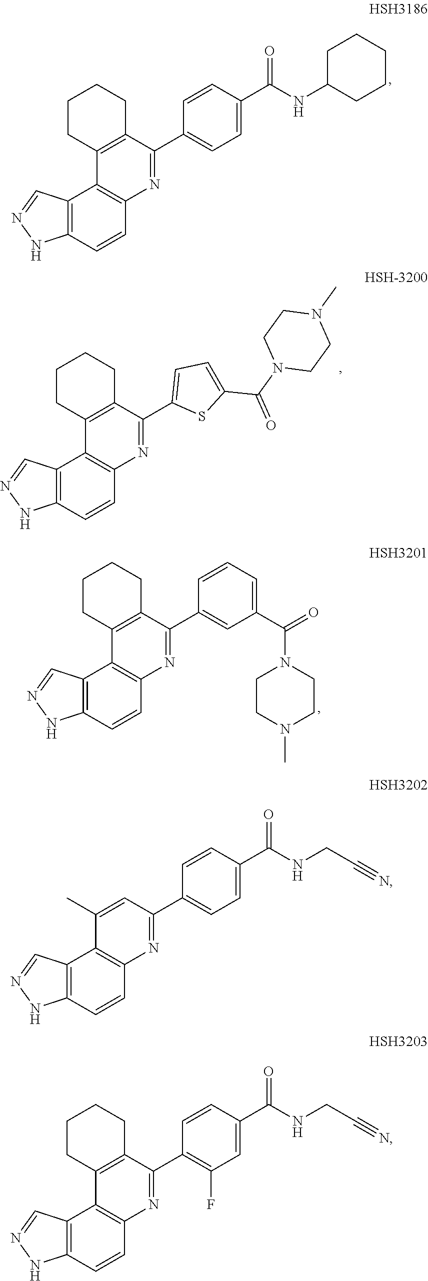 [SMILES: O=C(NC1CCCCC1)c1ccc(-c2nc3ccc4[nH]ncc4c3c3c2CCCC3)cc1 ; CN1CCN(C(=O)c2ccc(-c3nc4ccc5[nH]ncc5c4c4c3CCCC4)s2)CC1 ; CN1CCN(C(=O)c2cccc(-c3nc4ccc5[nH]ncc5c4c4c3CCCC4)c2)CC1 ; Cc1cc(-c2ccc(C(=O)NCC#N)cc2)nc2ccc3[nH]ncc3c12 ; N#CCNC(=O)c1ccc(-c2nc3ccc4[nH]ncc4c3c3c2CCCC3)c(F)c1]

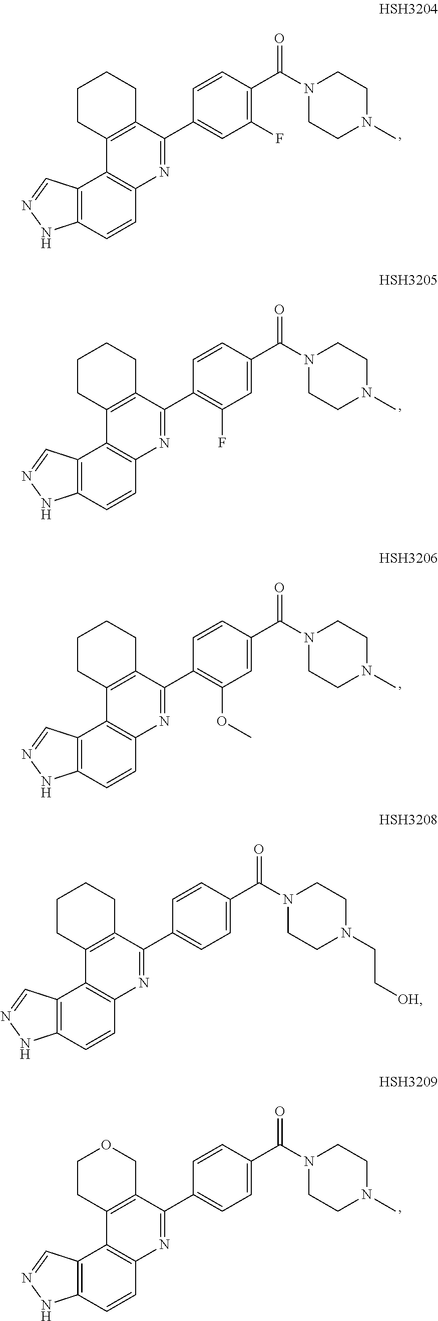 [SMILES: CN1CCN(C(=O)c2ccc(-c3nc4ccc5[nH]ncc5c4c4c3CCCC4)cc2F)CC1 ; CN1CCN(C(=O)c2ccc(-c3nc4ccc5[nH]ncc5c4c4c3CCCC4)c(F)c2)CC1 ; COc1cc(C(=O)N2CCN(C)CC2)ccc1-c1nc2ccc3[nH]ncc3c2c2c1CCCC2 ; O=C(c1ccc(-c2nc3ccc4[nH]ncc4c3c3c2CCCC3)cc1)N1CCN(CCO)CC1 ; CN1CCN(C(=O)c2ccc(-c3nc4ccc5[nH]ncc5c4c4c3COCC4)cc2)CC1]

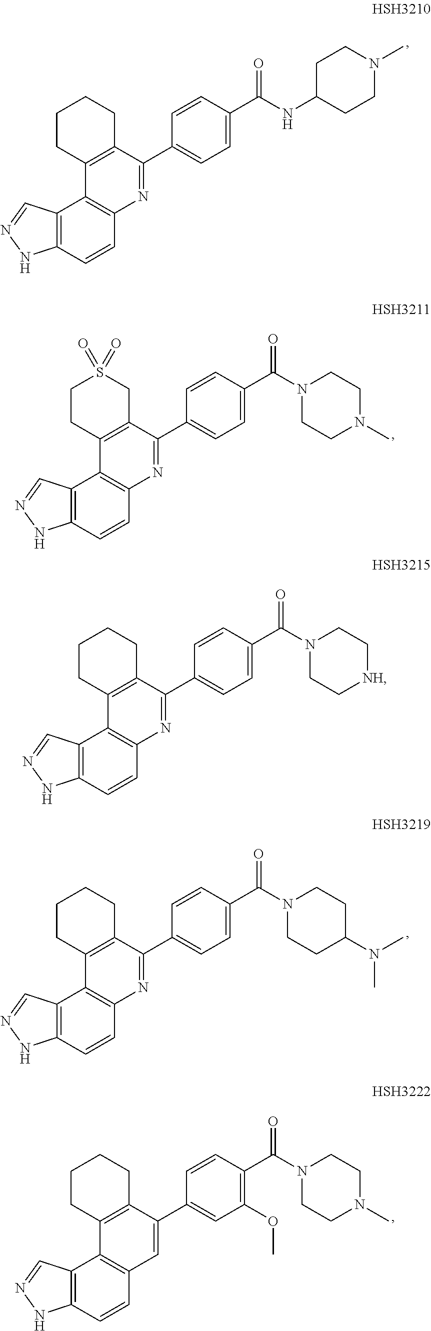 [SMILES: CN1CCC(NC(=O)c2ccc(-c3nc4ccc5[nH]ncc5c4c4c3CCCC4)cc2)CC1 ; CN1CCN(C(=O)c2ccc(-c3nc4ccc5[nH]ncc5c4c4c3CS(=O)(=O)CC4)cc2)CC1 ; O=C(c1ccc(-c2nc3ccc4[nH]ncc4c3c3c2CCCC3)cc1)N1CCNCC1 ; CN(C)C1CCN(C(=O)c2ccc(-c3nc4ccc5[nH]ncc5c4c4c3CCCC4)cc2)CC1 ; COc1cc(-c2cc3ccc4[nH]ncc4c3c3c2CCCC3)ccc1C(=O)N1CCN(C)CC1]

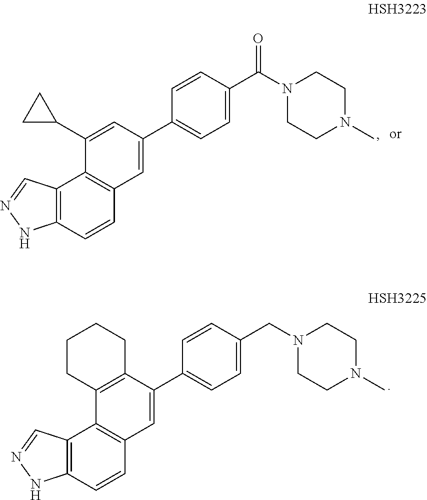 [SMILES: CN1CCN(C(=O)c2ccc(-c3cc(C4CC4)c4c(ccc5[nH]ncc54)c3)cc2)CC1 ; CN1CCN(Cc2ccc(-c3cc4ccc5[nH]ncc5c4c4c3CCCC4)cc2)CC1]

As noted above, the compounds can be CDK-specific inhibitors. In certain embodiments, the compounds are dual haspin and FLT3-specific inhibitors. The compound can bind to a CDK-receptor or both haspin and FLT3-receptors with specificity, thus reducing off-target interactions, for example, with growth factor receptors. “Binds with specificity,” “binds with high affinity,” or “specifically” or “selectively” binds, when referring to a small molecule/receptor or other binding pairs indicates that, under designated conditions, a specified compound or recognition region thereof binds to a particular receptor or site (e.g., a CDK inhibition site such as an ATP-binding catalytic site of the tyrosine kinase domain) and does not bind in a significant amount to other receptors present (e.g., off-target growth factor receptors). Specific binding or binding with high affinity can also mean, for example, that the binding compound, ligand, or binding composition binding to its target (e.g., a CDK inhibition or dual FLT3/haspin inhibition) with an affinity that is often at least 20% greater, at least 25% greater, at least 50% greater, or at least 100% (2-fold) greater than the affinity of the compound, ligand, or binding composition to bind with another receptor (e.g., a growth factor receptor).

In a typical embodiment, a molecule that specifically binds a target will have an affinity that is at least about 10⁶ liters/mol (Ko=10^{˜6} M), and preferably at least about 50 nM, as determined, for example, by Scatchard analysis. The binding specificity of the compound to a CDK receptor or to both haspin and FLT3 receptors is determined using standard techniques known in the art and described herein.

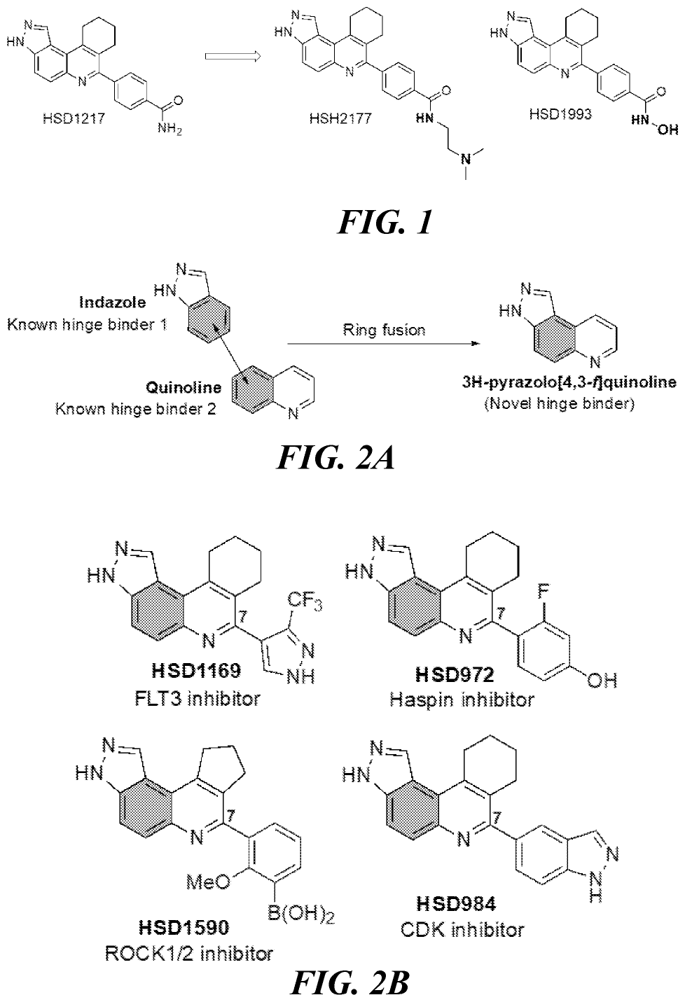

In certain embodiments, the N-(2-(dimethylamino)ethyl)benzamide or hydroxamic acid derivatives hereof can dramatically improve activity against kinases, including CDKs. FIG. 1 shows the modification of previously known compound HSD1217 to Compounds HSH2177 and HSD1993 hereof. While HSD1217 inhibits CDK2 with IC₅₀ value of 185 nM, Compound HSH2177 can inhibit CDK2 with an IC₅₀ value of 7 nM and Compound HSD1993 can inhibit CDK2 with an IC₅₀ value of 4 nM. Furthermore, Compound HSD2177 and Compound HSD1993 inhibit CDK12/CyclinK with IC₅₀ values of 27 nM and 9 nM, respectively. Under similar experimental conditions, THZ531 and dinaciclib inhibited CDK12 with IC₅₀ values of 60 nM and 4 nM, respectively.

The present inventors have previously shown that the “fusion” of two known kinase hinge binders, indazole and quinoline, which are commonly found in U.S. Food & Drug Administration-(“FDA”) approved drugs, such as axitinib and erlotinib, can lead to 3H-pyrazolo[4,3-f]quinoline (FIG. 2A) which can bind to the hinge region of kinases. Dayal et al., 3H-pyrazolo[4,3-f]quinoline-based kinase inhibitors inhibit the proliferation of acute myeloid leukemia cells in vivo, J Medicinal Chemistry 64 (15): 10981-10996 (2021). Remarkably, the substituent at position 7 of the 3H-pyrazolo[4,3-f]quinoline moiety can dictate which kinases are potently inhibited. For example, pyrazole substitution at position 7 can lead to inhibitors that bind to mutant FLT3 with picomolar K_{d} values, while phenyl boronic acid substituents can lead to ultrapotent ROCK1/2 inhibitors. Dayal et al., Dual FLT3/TOPK inhibitor with activity against FLT3-ITD secondary mutations potently inhibits acute myeloid leukemia cell lines, Future Medicinal Chemistry 10 (7): 823-835 (2018); Dayal et al., Potently inhibiting cancer cell migration with novel 3H-pyrazolo[4,3-f]quinoline boronic acid ROCK inhibitors, European J Medicinal Chemistry 180:449-456 (2019) (FIG. 2B).

Benzo[d]thiazolyl substituted compounds like SHD992 can be potent CDK inhibitors, while phenol containing compounds such as HSD972 (see FIG. 2B) can be excellent haspin inhibitors (HSD972 haspin IC₅₀=14 nM). Opoku-Temeng et al., Tetrahydro-3H-pyrazolo[4,3-a]phenanthridine-based CDK inhibitor, Chemical Communications 54 (36): 4521-4524 (2018); Opoku-Temeng et al., 3H-pyrazolo[4,3-f]quinoline haspin kinase inhibitors and anti-cancer properties, Bioorganic Chemistry 78:418-426 (2018). In addition to the 3H-pyrazolo[4,3-f]quinoline core, certain other analogous systems, such as pyrazolo[3,4-h]quinoline scaffold, can also have anticancer activities. Nguyen et al., Induction of paraptotic cell death in breast cancer cells by a novel pyrazolo[3,4-h]quinoline derivative through ROS production and endoplasmic reticulum stress, Antioxidants 11 (1): 117 (2022).

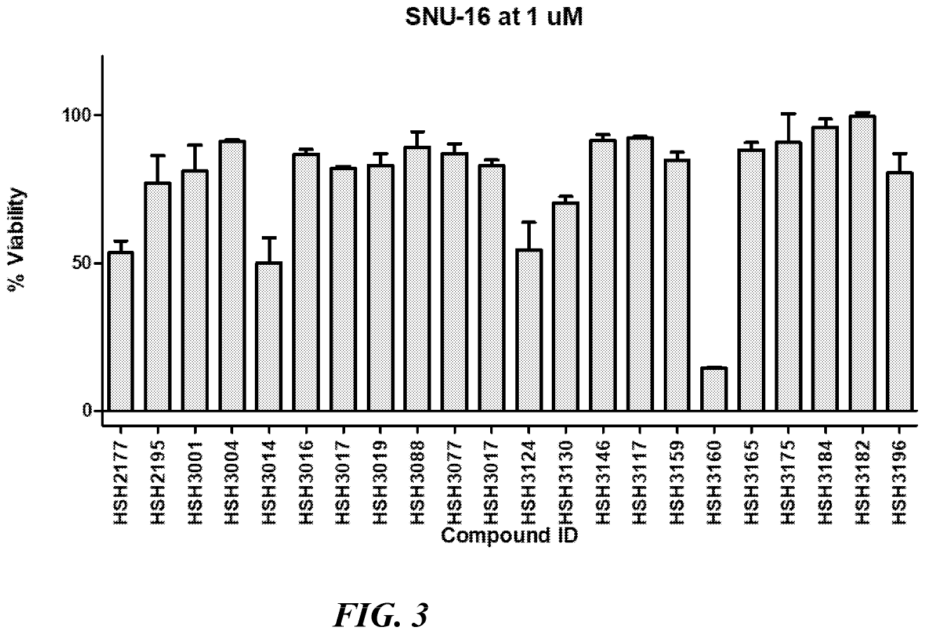

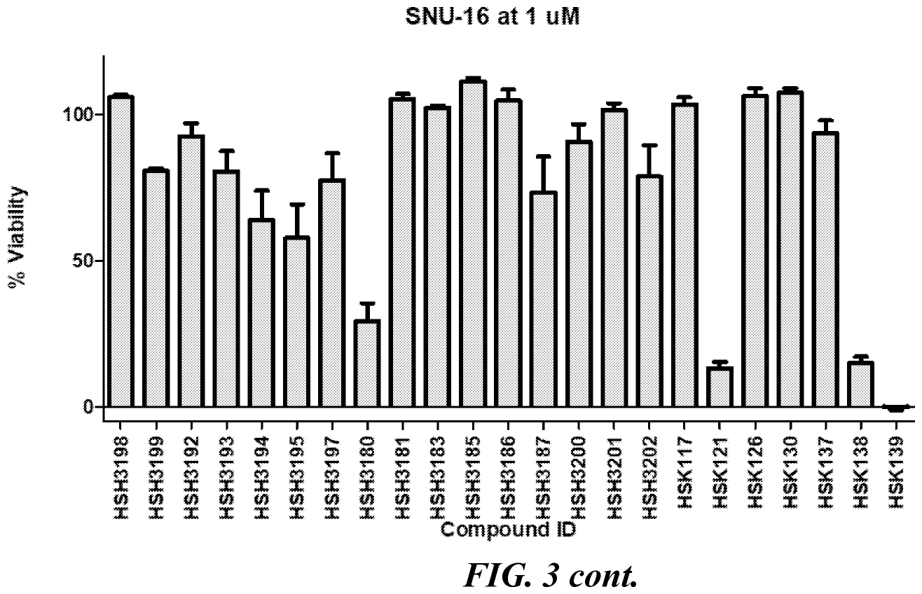

While both HDS972 and HSD1169 (see FIG. 2B) contain the 3H-pyrazolo[4,3-f]quinoline moiety, they inhibit haspin or FLT3, respectively; not both. In certain embodiments, the compounds hereof comprise a replacement of the phenol moiety at position-7 with benzamide (Compound HSD1217, see FIG. 3A), which inhibits haspin more potently than HDS972. In certain embodiments, the compounds hereof are derivatives of Compound HSD1217, which have been optimized for dual FLT3/haspin binding.

The compounds described herein may contain one or more chiral centers or may otherwise be capable of existing as multiple stereoisomers. It is to be understood that in one embodiment, the invention described herein is not limited to any particular stereochemical requirement, and that the compounds, and compositions, methods, uses, and medicaments that include them may be optically pure, or may be any of a variety of stereoisomeric mixtures, including racemic and other mixtures of enantiomers, other mixtures of diastereomers, and the like. It is also to be understood that such mixtures of stereoisomers may include a single stereochemical configuration at one or more chiral centers, while including mixtures of stereochemical configuration at one or more other chiral centers.

Similarly, the compounds described herein may include geometric centers, such as cis, trans, E, and Z double bonds. It is to be understood that in another embodiment, the invention described herein is not limited to any particular geometric isomer requirement, and that the compounds, and compositions, methods, uses, and medicaments that include them may be pure, or may be any of a variety of geometric isomer mixtures. It is also to be understood that such mixtures of geometric isomers may include a single configuration at one or more double bonds, while including mixtures of geometry at one or more other double bonds.

It is contemplated that the compounds hereof can be provided in prodrug form. The term “prodrug” means an inactive derivative of a parent compound/drug that can hydrolyze, oxidize, or otherwise react under biological conditions (in vitro or in vivo) to provide an active compound, particularly a compound hereof. Prodrugs can be created to overcome one or more barriers to the effective use of the underlying active compound such as instability and/or possible toxicity barriers that exist with the active compound. Examples of prodrugs include, but are not limited to, derivatives and metabolites of a compound that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Specific prodrugs of compounds with carboxyl functional groups are the lower alkyl esters of the carboxylic acid. The carboxylate esters are conveniently formed by esterifying any of the carboxylic acid moieties present on the molecule. In certain embodiments, a prodrug form of a compound hereof can comprise a protecting group that will, in addition to protecting the compound from oxidation or the like, permit the targeting of specific sites within a subject's body (e.g., a tumor microenvironment). Prodrugs can typically be prepared using well-known methods, such as those described by Burger's Medicinal Chemistry and Drug Discovery 6th ed. (Donald J. Abraham ed., 2001, Wiley) and Design and Application of Prodrugs (H. Bundgaard ed., 1985, Harwood Academic Publishers GmbH).

### PROTAC Conjugates

PROTAC conjugates comprising the compounds hereof are also provided. A PROTAC conjugate can have a chemical structure of Formula (X):

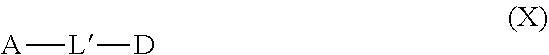

wherein A is a compound described herein (e.g., a kinase inhibitor described herein); L′ is a linker or absent; and D is a ubiquitin pathway protein binding moiety.

While inhibitors of proteins can block or reduce protein activity in a cell, protein degradation in a cell can also reduce activity or remove altogether the target protein. Utilizing a cell's protein degradation pathway can, therefore, provide additional means for reducing or removing protein activity. One of the cell's major degradation pathways is known as the ubiquitin-proteasome system. In this system, a protein is marked for degradation by the proteasome by ubiquitinating the protein. The ubiquitination of the protein can be accomplished by a ubiquitin pathway protein binding moiety that binds to a protein and adds ubiquitin molecules to the protein.

To harness this degradation pathway, PROTACs have been developed. The term “PROTAC” refers to proteolysis-targeting chimera molecules comprising a ubiquitin pathway protein binding moiety, optionally a linker, and targeting moiety. PROTACs bring together the ubiquitin pathway protein binding moiety (e.g., an E3 ubiquitin ligase) with a protein that is to be targeted for degradation. To facilitate a protein for degradation by the proteasome, the PROTAC is comprised of a group that binds to an ubiquitin ligase and a group that binds to the protein targeted for degradation (optionally these groups are connected with a linker). This molecular construct can bring the ubiquitin pathway protein binding moiety in proximity with the protein so that it is ubiquitinated and marked for degradation.

The ubiquitin pathway protein binding moiety is any suitable structure that recognizes and binds to a ubiquitin pathway protein. In general, a ubiquitin pathway protein is any entity or complex that is capable of catalyzing or causing to catalyze the transfer of a ubiquitin or ubiquitin-like modifying polypeptide (e.g., Nedd8, APG12, or ISG15/UCRP) to another protein. In certain embodiments, the ubiquitin pathway protein is a ubiquitin protein ligase or E3 protein. There are at least 100 distinct E3 proteins encoded by the human genome. Winston et al., A Family of Mammalian F-Box Proteins, Current Biology 9 (20): 1180-1182 (1999).

In certain embodiments, a ubiquitin pathway protein is a protein that is involved in or a component of a ubiquitin-like pathway, which transfers ubiquitin-like modifying polypeptides (e.g., SUMO, Nedd8, APG12, or ISG15/UCRP). Components of a ubiquitin-like modifying pathway are usually homologs of a ubiquitin pathway. For example, the ubiquitin-like pathway for SUMO includes a homolog of a ubiquitin protein activating enzyme or E1 protein, ubiquitin protein conjugating enzyme or E2 protein and ubiquitin ligase or E3 protein.

In certain embodiments, a ubiquitin pathway protein binding moiety is any suitable ligand to a ubiquitin pathway protein, for example, ubiquitin protein ligase or E3 protein or homologs thereof. In certain embodiments, a ubiquitin pathway protein binding peptide, domain or region of a ligand to a ubiquitin pathway protein. In certain embodiments, a ubiquitin pathway protein binding moiety recognizes and binds to a ubiquitin pathway protein in a regulated manner.

The A component is a compound hereof that binds to a target protein intended to be degraded (e.g., a targeted kinase inhibitor such as CDK, FLT3, or haspin kinase). The term “protein” includes oligopeptides and polypeptide sequences of sufficient length that they can bind to the A component.

Specific A groups are any of the compounds described herein.

The compound and the ubiquitin pathway protein binding moiety can be optionally connected with a linker. The linker can be any suitable linker. The linker can comprise atoms selected from C, N, O, S, Si, and P; C, N, O, S, and P; or C, N, O, and S. The linker can have a backbone that ranges in length, such that there can be as few as two atoms in the backbone of the linker to as many as 100 or more contiguous atoms in the backbone of the linker. The “backbone” of the linker is the shortest chain of contiguous atoms forming a covalently bonded connection between A and D. In some embodiments, a polyvalent linker has a branched backbone, with each branch serving as a section of backbone linker until reaching a terminus.

For example, the linker can have a chain length of at least about 7 atoms. In some embodiments, the linker is at least about 10 atoms in length. In some embodiments, the linker is at least about 14 atoms in length. In some embodiments, the linker is between about 7 and about 31 atoms (such as, about 7 to 31, 7 to about 31, or 7 to 31), between about 7 and about 24 atoms (such as, about 7 to 24, 7 to about 24, or 7 to 24), or between about 7 and about 20 atoms (such as, about 7 to 20, 7 to about 20, or 7 to 20) atoms. In some embodiments, the linker is between about 14 and about 31 atoms (such as, about 14 to 31, 14 to about 31, or 14 to 31), between about 14 and about 24 atoms (such as, about 14 to 24, 14 to about 24, or 14 to 24), or between about 14 and about 20 atoms (such as, about 14 to 20, 14 to about 20, or 14 to 20). In some embodiments, the linker has a chain length of at least 7 atoms, at least 14 atoms, at least 20 atoms, at least 25 atoms, at least 30 atoms, or at least 40 atoms; or from 1 to 15 atoms, 1 to 5 atoms, 5 to 10 atoms, 5 to 20 atoms, 10 to 40 atoms, or 25 to 100 atoms.

The linker can comprise at least one carbon-carbon bond and/or at least one amide bond. The linker can comprise one or more L- or D-configurations, natural or unnatural amino acids, a PEG monomer, a PEG oligomer, a PEG polymer, or a combination of any of the foregoing. For a linker that comprises one or more PEG units, all carbon and oxygen atoms of the PEG units are part of the backbone, unless otherwise specified.

In certain embodiments, the linker is a group comprising one or more covalently connected structural units.

In certain embodiments, the linker group is optionally substituted (poly)ethyleneglycol having between 1 and about 100 ethylene glycol units, between about 1 and about 50 ethylene glycol units, between 1 and about 25 ethylene glycol units, between about 1 and 10 ethylene glycol units, between 1 and about 8 ethylene glycol units and 1 and 6 ethylene glycol units, between 2 and 4 ethylene glycol units, and/or optionally substituted alkyl groups interdispersed with optionally substituted, O, N, S, P or Si atoms. In certain embodiments, the linker is substituted with an aryl, phenyl, benzyl, alkyl, alkylene, or heterocycle group. In certain embodiments, the linker is asymmetric. In certain embodiments, the linker is symmetrical.

Alternatively, or in addition to chain length, in some embodiments, the linker can have suitable substituents that affect hydrophobicity or hydrophilicity. Thus, for example, the linker can have a hydrophobic side chain group, such as an alkyl, cycloalkyl, aryl, arylalkyl, or like group, each of which is optionally substituted. If the linker includes one or more amino acids, the linker can contain hydrophobic amino acid side chains, such as one or more amino acid side chains from Phe and Tyr, including substituted variants thereof, and analogs and derivatives of such side chains.

The linker can comprise a spacer (e.g., be conjugated with and/or include a spacer). The spacer can be any suitable spacer. A spacer of the linker can comprise hydrophilic, hydrophobic, amphipathic, non-peptidic, peptidic, and/or aromatic monomers. The length of a spacer can range from 1 to 30 (e.g., 1 to 30 carbon atoms, a PEG with 1-30 units, etc.). Examples of hydrophilic spacers include, but are not limited to, polyethylene glycol polymers and derivatives thereof. Examples of hydrophobic spacers include, but are not limited to, pure or mixed branched hydrocarbons, fluorocarbons, alkane, alkene, and/or alkyne polymers. Examples of amphipathic spacers include, but are not limited to, pure or mixed phospholipids and/or derivatives thereof. Examples of peptidic spacers include, but are not limited to, pure and mixed single, branched, L- or D-configurations, essential, nonessential, natural, and unnatural amino acids and derivatives thereof. Examples of aromatic spacers include, but are not limited to, pure and mixed repeated quinoids.

In some embodiments the linker is formed via click chemistry/click chemistry-derived synthetic methods. Those of skill in the art understand that the terms “click chemistry” and “click chemistry-derived” generally refer to a class of small molecule reactions commonly used in conjugation, allowing the joining of substrates of choice with specific molecules. Click chemistry is not a single specific reaction but describes a way of generating products that follow examples in nature, which also generate substances by joining small modular units. In many applications click reactions join a biomolecule and a reporter molecule. Click chemistry is not limited to biological conditions; the concept of a “click” reaction has been used in pharmacological and various biomimetic applications. However, they have been made notably useful in the detection, localization and qualification of biomolecules.

Click reactions can occur in one pot, typically are not disturbed by water, can generate minimal byproducts, and are “spring-loaded”—characterized by a high thermodynamic driving force that drives it quickly and irreversibly to high yield of a single reaction product, with high reaction specificity (in some cases, with both regio- and stereo-specificity). These qualities make click reactions suitable to the problem of isolating and targeting molecules in complex biological environments. In such environments, products accordingly need to be physiologically stable and any byproducts need to be non-toxic (e.g., for in vivo systems).

### Salts

The compounds and conjugates can be presented as a pharmaceutically acceptable salt. The term “pharmaceutically acceptable salt” refers to those salts whose counter ions can be used in pharmaceuticals. In various embodiments, such salts include, but are not limited to 1) acid addition salts, which can be obtained by reaction of the free base of the parent compound with inorganic acids such as hydrochloric acid, hydrobromic acid, nitric acid, phosphoric acid, sulfuric acid, and perchloric acid and the like, or with organic acids such as acetic acid, oxalic acid, (D) or (L) malic acid, maleic acid, methane sulfonic acid, ethanesulfonic acid, p-toluenesulfonic acid, salicylic acid, tartaric acid, citric acid, succinic acid or malonic acid and the like; or 2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, trimethamine, N-methylglucamine, and the like. Pharmaceutically acceptable salts are well-known to those skilled in the art, and any such pharmaceutically acceptable salt is contemplated in connection with the embodiments described herein.

Pharmaceutically acceptable salts can be synthesized from the parent compound which contains a basic or acidic moiety by conventional chemical methods. In some instances, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, the disclosure of which is hereby incorporated by reference.

In various embodiments, suitable acid addition salts are formed from acids which form non-toxic salts. Illustrative examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

In various embodiments, suitable base salts are formed from bases which form non-toxic salts. Illustrative examples include the arginine, benzathine, calcium, choline, diethylamine, diolamine, glycine, lysine, magnesium, meglumine, olamine, potassium, sodium, tromethamine and zinc salts. Hemisalts of acids and bases also can be formed, for example, hemisulphate and hemicalcium salts.

In each embodiment hereof, it will be understood that the formulae include and represent not only all pharmaceutically acceptable salts of the compounds and conjugates, but also include any and all hydrates and/or solvates of the compound formulae or salts thereof. The term “solvate” means a compound, or a salt thereof, that further includes a stoichiometric or non-stoichiometric amount of solvent bound by non-covalent intermolecular forces. Where the solvent is water, the solvate is a hydrate.

It is to be appreciated that certain functional groups, such as the hydroxy, amino, and like groups form complexes and/or coordination compounds with water and/or various solvents, in the various physical forms of the compounds. Accordingly, the formulae are to be understood to include and represent those various hydrates and/or solvates.

### Compositions, Routes of Administration, and Dosing

Further provided is a composition comprising an above-described compound (e.g., a compound of formula I, formula IA, formula IB, or formula II) or conjugate (e.g., a conjugate of formula X) and a pharmaceutically acceptable carrier or excipient. The term “composition” generally refers to any product comprising more than one ingredient, including the compound or conjugate. The compositions can be prepared from isolated compounds or conjugates or from salts, solutions, hydrates, solvates, and other forms of the compounds and/or conjugates.

The term “pharmaceutically acceptable carrier” means one or more compatible solid or liquid fillers, diluents or encapsulating substances which are suitable for administration to a human or other vertebrate animal. The term “carrier” denotes an organic or inorganic ingredient, natural or synthetic, with which the active ingredient is combined to facilitate the application. The carrier can be an excipient. The choice of carrier can depend on factors such as the particular mode of administration, the effect of the carrier on solubility and stability, and the nature of the dosage form. Pharmaceutical compositions suitable for the delivery of compounds as described herein and methods for their preparation may be found, for example, in Remington: The Science & Practice of Pharmacy, 21st edition (Lippincott Williams & Wilkins, 2005).

The components of the compositions also can be commingled with the compound, and with each other, in a manner such that there is no interaction which would substantially impair the desired pharmaceutical efficiency.

The composition can comprise cremophor, polysorbate, nanoparticles, a polymer, or a hydrogel, for example. In certain embodiments, the pharmaceutical composition comprises a plurality of compounds and a pharmaceutically acceptable carrier. In certain embodiments, a pharmaceutical composition further comprises at least one additional pharmaceutically active agent. The at least one additional pharmaceutically active agent can be an agent useful in the treatment of a cancer.

Pharmaceutical compositions can be prepared by combining one or more compounds or conjugates with a pharmaceutically acceptable carrier and, optionally, one or more additional ingredients (e.g., pharmaceutically active ingredients). The formulations can be administered in pharmaceutically acceptable solutions, which can routinely contain pharmaceutically acceptable concentrations of salt, buffering agents, preservatives, compatible carriers, adjuvants, and optionally other therapeutic ingredients.

A pharmaceutically acceptable carrier can include any and all solvents, dispersion media, coatings, antibacterial and antifungal agents, isotonic and absorption delaying agents, and the like, and combinations thereof, that are physiologically compatible. The carrier can be suitable for parenteral administration. Pharmaceutically acceptable carriers include sterile aqueous solutions or dispersions and sterile powders for the extemporaneous preparation of sterile injectable solutions or dispersions. Examples of such carriers (or excipients) include, but are not limited to, calcium carbonate, calcium phosphate, various sugars, starches, cellulose derivatives, gelatin, and polymers such as polyethylene glycols. One or more other active agents also can be incorporated into a pharmaceutical composition.

The composition can be formulated as a liquid, e.g., a suspension or a solution. A liquid formulation can comprise water, ethanol, polyethylene glycol, propylene glycol, methylcellulose, or a suitable oil, and one or more emulsifying agents and/or suspending agents. A liquid formulation can be prepared by the reconstitution of a solid.

Pharmaceutical formulations (e.g., for parenteral administration) include aqueous solutions of the active compounds in water-soluble form. Additionally, suspensions of the active compounds can be prepared as appropriate oily injection suspensions. An aqueous suspension can contain a compound, alone or in further combination with one or more other active agents, in admixture with an appropriate excipient. Excipients include suspending agents, such as sodium carboxymethylcellulose, methylcellulose, hydroxypropylmethylcellulose, sodium alginate, polyvinylpyrrolidone, gum tragacanth and gum acacia; dispersing or wetting agents, such as a naturally-occurring phosphatide, e.g., lecithin; a condensation product of an alkylene oxide with a fatty acid, e.g., polyoxyethylene stearate; a condensation product of ethylene oxide with a long-chain aliphatic alcohol, e.g., heptadecaethyleneoxcycetanol; a condensation product of ethylene oxide with a partial ester derived from fatty acids and a hexitol, such as polyoxyethylene sorbitol monooleate; or a condensation product of ethylene oxide with a partial ester derived from fatty acids and hexitol anhydrides, e.g., polyoxyethylene sorbitan monooleate. The aqueous suspension also can contain one or more preservatives, e.g., ascorbic acid or ethyl, n-propyl, or p-hydroxybenzoate, and one or more coloring agents. In certain embodiments, an aqueous suspension can further comprise suitable lipophilic solvents or vehicles including fatty oils such as sesame oil, or synthetic fatty acid esters, such as ethyl oleate or triglycerides, or liposomes. Optionally, the suspension can also contain suitable stabilizers or agents that increase the solubility of the compounds to allow for the preparation of highly concentrated solutions.

Alternatively, the pharmaceutical compositions can be in powder form for constitution with a suitable vehicle, e.g., sterile pyrogen-free water, before use. Dispersible powders and granules suitable for preparation of an aqueous suspension by the addition of water can provide the active ingredient in admixture with a suspending agent, a dispersing or wetting agent, and one or more preservatives. Additional excipients, for example, coloring agents, also can be present.

Suitable emulsifying agents include naturally occurring gums, e.g., gum acacia or gum tragacanth; naturally occurring phosphatides, e.g., soybean lecithin; and esters, including partial esters derived from fatty acids and hexitol anhydrides, e.g., sorbitan mono-oleate, and condensation products of partial esters with ethylene oxide, e.g., polyoxyethylene sorbitan monooleate. Isotonic agents, e.g., sugars, polyalcohols, such as mannitol, sorbitol, or sodium chloride, can be included in the composition. Prolonged absorption of injectable compositions can be achieved by including in the composition one or more agents to delay absorption, e.g., monostearate salts and gelatin.

For use in therapy or treatment, an effective amount of the compound or composition can be administered to a subject by any mode that delivers the compound as desired. Administering a composition can be accomplished by any means known to the skilled artisan. Routes of administration include, but are not limited to, intravenous, intramuscular, intraperitoneal, intravesical (urinary bladder), oral, subcutaneous, direct injection, mucosal (e.g., topical to eye), inhalation, and topical.

Colorants and/or flavoring agents can be included. For example, the compound can be formulated (such as by liposome or microsphere encapsulation) and then further contained within an edible product, such as a refrigerated beverage containing colorants and flavoring agents.

Illustrative formats for oral administration include, but are not limited to, tablets, capsules, elixirs, syrups, and the like.

In certain embodiments, a compound can be administered directly into the blood stream, into muscle, or into an internal organ. Suitable routes for such parenteral administration include intravenous, intraarterial, intraperitoneal, intrathecal, epidural, intracerebroventricular, intraurethral, intrasternal, intracranial, intratumoral, intramuscular, intranasal, and subcutaneous. Suitable means for parenteral administration include needle (including microneedle) injectors, needle-free injectors, and infusion techniques. Where it is desirable to deliver the compound(s) and/or compositions systemically, the compound(s) and/or composition can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

Parenteral formulations are typically aqueous solutions that can contain carriers or excipients, such as salts, carbohydrates, and buffering agents (preferably at a pH of 3-9), but, for some applications, they may be more suitably formulated as a sterile non-aqueous solution or as a dried form to be used in conjunction with a suitable vehicle, such as sterile, pyrogen-free water.

A liquid formulation can be adapted for parenteral administration of a compound. The preparation of parenteral formulations under sterile conditions, for example, by lyophilization under sterile conditions, can readily be accomplished using standard pharmaceutical techniques well-known to those skilled in the art. The solubility of a compound can be increased by the use of appropriate formulation techniques, such as the incorporation of solubility-enhancing agents.

Formulations for parenteral administration can be formulated for immediate and/or modified release. A compound can be administered in a time-release formulation, for example in a composition which includes a slow-release polymer. The compound can be prepared with a carrier that will protect it against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PGLA). Methods for the preparation of such formulations are generally known to those skilled in the art.

Sterile injectable solutions can be prepared by incorporating the compound(s), alone or in further combination with one or more other active agents, in the required amount in an appropriate solvent with one or a combination of ingredients described above, as required, followed by filtered sterilization. Typically, dispersions are prepared by incorporating the compound into a sterile vehicle, which contains a dispersion medium and any additional ingredients of those described above. In the case of sterile powders for the preparation of sterile injectable solutions, the preferred methods of preparation are vacuum-drying and freeze-drying, which yield a powder of the active ingredients plus any additional desired ingredient from a previously sterile-filtered solution thereof, or the ingredients can be sterile-filtered together.

The pharmaceutical composition can be formulated as a solution, microemulsion, liposome, or other ordered structure suitable to high drug concentration. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating, such as lecithin, by the maintenance of the required particle size in the case of dispersion, and by the use of surfactants.

A compound, or a pharmaceutical composition comprising a compound, can be continuously administered, where appropriate.

### Methods of Treatment and Uses

Methods of treating a kinase-mediated disease are provided. In certain embodiments, the kinase-mediated disease is a cancer. The method can comprise administering to a subject (e.g., a subject experiencing or at risk of experiencing a kinase-mediated disease such as cancer) an effective amount of an above-described compound, an above-described conjugate, a pharmaceutically acceptable salt, N-oxide, hydrate, solvate, tautomer, or optical isomer of the compound or conjugate, or any of the pharmaceutical compositions hereof. In certain embodiments, the compound has a structure of formula I:

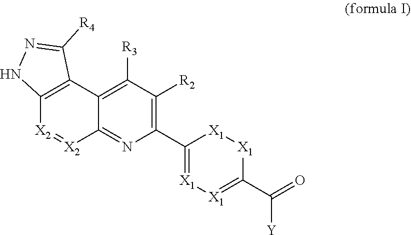

or is a pharmaceutically acceptable salt thereof, wherein:
X₁ is each independently N, CH, or a C-halogen;
X₂ is each independently N, CH, a C-halogen, C—CHₙ₁Xₘ where n1+m=3 and X is a halogen, C—OCF₃, C—OCHF₂, C—OMe, C—Me, C—Et, C-tBu, C-iPr, C—N-alkyl, C—N-heteroalkyl, C—O-alkyl, or C—O-heteroalkyl;
R₂, R₃, and R₄ are each independently an alkyl, cycloalkyl, heteroalkyl, or heterocycloalkyl; and
Y is

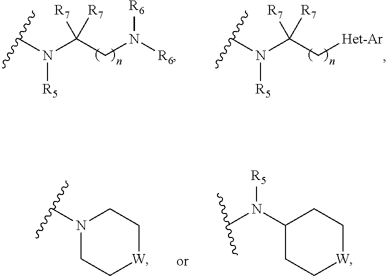

wherein:
n is 1 to 5,
R₅ and R₆ are each independently H, an alkyl, or a heteroalkyl, and R₆ can optionally form a cyclic structure,
R₇ is each independently H or an alkyl,
Het-Ar is a heteroaromatic ring, and
W is NH, NMe, NEt, NCH₂CH₂OH, NCH₂CH₂OMe, O, SO, or SO₂,
with the proviso that Y is not NH₂.

In certain embodiments, the compound has a structure of formula IA:

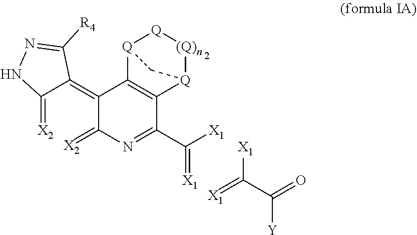

or is a pharmaceutically acceptable salt thereof, wherein:
X₁ is each independently N, CH, or a C-halogen;
X₂ is each independently N, CH, a C-halogen, C—CHₙ₁Xₘ where n1+m=3 and X is a halogen, C—OCF₃, C—OCHF₂, C—OMe, C—Me, C—Et, C-tBu, C-iPr, C—N-alkyl, C—N-heteroalkyl, C—O-alkyl, or C—O-heteroalkyl;
R₄ is an alkyl, cycloalkyl, heteroalkyl, or heterocycloalkyl;
Y is

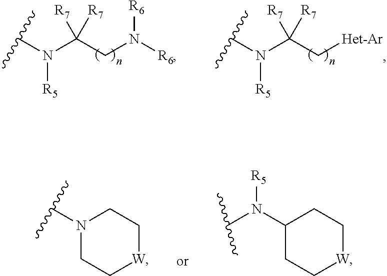

wherein:
n is 1 to 5,
R₅ and R₆ are each independently H, an alkyl, or a heteroalkyl, and R₆ can optionally form a cyclic structure,
R₇ is each independently H or an alkyl,
Het-Ar is a heteroaromatic ring, and
W is NH, NMe, NEt, NCH₂CH₂OH, NCH₂CH₂OMe, O, SO, or SO₂,
with the proviso that Y is not NH₂.
n2 is 1, 2 or 3; and
Q is each independently O, NH, substituted N, CH₂, or substituted C, S, SO, or SO₂; and
each Q, taken together, form a 6-, 7-, or 8-membered ring or bicyclic ring.

In certain embodiments, the compound has a structure of formula IB:

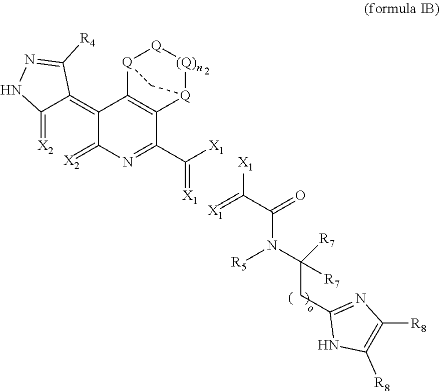

or is a pharmaceutically acceptable salt thereof, wherein:
X₁ is each independently N, CH, or a C-halogen;
X₂ is each independently N, CH, a C-halogen, C—CHₙ₁Xₘ where n1+m=3 and X is a halogen, C—OCF₃, C—OCHF₂, C—OMe, C—Me, C—Et, C-tBu, C-iPr, C—N-alkyl, C—N-heteroalkyl, C—O-alkyl, or C—O-heteroalkyl;
R₄ is an alkyl, cycloalkyl, heteroalkyl, or heterocycloalkyl;
R₅ is H, an alkyl, or a heteroalkyl;
R₇ is each independently H or an alkyl,
is 0-2; and
each R₈ is independently H, an alkyl, or a heteroalkyl.

In certain embodiments, the compound has a structure of formula II:

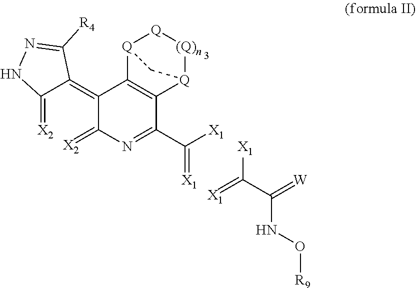

or is a pharmaceutically acceptable salt thereof, wherein:
X₁ is each independently N, CH, or a C-halogen;
X₂ is each independently N, CH, a C-halogen, C—CHₙ₁Xₘ where n1+m=3 and X is a halogen, C—OCF₃, C—OCHF₂, C—OMe, C—Me, C—Et, C-tBu, C-iPr, C—N-alkyl, C—N-heteroalkyl, C—O-alkyl, or C—O-heteroalkyl;
Q is each independently O, NH, substituted N, CH₂, or substituted C, S, SO, or SO₂, and each Q, when taken together, form a 6-, 7-, or 8-membered ring or bicyclic ring;
n3 is 1, 2, or 3;
R₄ is an alkyl, cycloalkyl, heteroalkyl, or heterocycloalkyl;
W is O, NH, or NMe; and
R₉ is an alkyl or a heteroalkyl, with the proviso that R₉ is not H.

The kinase-mediated disease can be selected from the group consisting of cancer, inflammatory disease, diabetes, and a neurological disease. In certain embodiments, the kinase-mediated disease is a cancer. The cancer can be acute myeloid leukemia (AML). The cancer can be chronic myeloid leukemia. The cancer can be gastric carcinoma. The cancer can be a resistant cancer. The cancer can be selected from the group consisting of AML, chronic myeloid leukemia, ovarian cancer, cervical cancer, pancreatic cancer, breast cancer, brain cancer, skin cancer, lung cancer, prostate cancer, lymphoma, leukemia, colon cancer, gastric carcinoma, head cancer, neck cancer, thyroid cancer, kidney cancer, liver cancer, and stomach cancer.

In certain embodiments, the compound or conjugate administered to the subject comprises Compound HSH2177. In certain embodiments, the compound or conjugate administered to the subject comprises Compound HSH2177 and administering an effective amount to the subject inhibits a CDK in the subject at an IC₅₀ of about 50 nM or lower (such as 50 nM or lower). In certain embodiments, the compound or conjugate administered to the subject comprises Compound 205. In certain embodiments, the compound or conjugate administered to the subject comprises Compound 205 and administering an effective amount to the subject inhibits both FLT3 and haspin kinase with specificity in the subject.

In certain embodiments of the method, administering the effective amount to the subject inhibits a CDK (e.g., with specificity) in the subject at an IC₅₀ of about 50 nM or lower (such as 50 nM or lower). In certain embodiments of the method, administration of the compound or pharmaceutical composition inhibits a CDK (e.g., with specificity) at an IC₅₀ of about 30 nM or lower (e.g., 30 nM or lower, 29 nM or lower, 28 nM or lower, 27 nM or lower, 25 nM or lower, . . . 10 nM or lower, or less than 5 nM). Administering the effective amount to the subject can inhibit both FLT3 and haspin (e.g., with dual specificity). In certain embodiments, administering the effective amount of Compound 205 or an analog thereof, for example, dually inhibits both FLT3 and haspin kinase at an IC₅₀ of about 50 nM or lower (such as IC₅₀ of 50 nM or lower).

The terms “treat,” “treating,” “treated,” or “treatment” (with respect to a disease state or disorder) is an approach for obtaining beneficial or desired results including and preferably clinical results and includes, but is not limited to, one or more of the following: improving a condition associated with a disease, curing a disease, lessening severity of a disease or otherwise suppressing the disease, delaying progression of a disease, alleviating one or more symptoms associated with a disease, reducing the severity of a disease, inhibiting the onset of a disease, inhibiting the progression of a disease, increasing the quality of life of one suffering from a disease, prolonging survival and/or prophylactic or preventative treatment.

As used herein, an “effective amount” refers to any amount of a compound or conjugate with respect to use in treatment, refers to an amount of the compound or conjugate in a preparation which, when administered as part of a desired dosage regimen (to a subject such as a human) that is sufficient to achieve a desired biological effect (e.g., alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment).

In certain embodiments, the method can further comprise administering to the subject a second therapy comprising an effective amount of a chemotherapeutic agent (e.g., when the disease state is a cancer), an immunotherapeutic agent (e.g., when the disease state is an inflammatory disease), or a hormone therapeutic agent. In certain embodiments the second therapy can comprise a radiation therapy.

It will be understood that in the methods described herein, the individual components of a co-administration, or combination can be administered by any suitable means, contemporaneously, simultaneously, sequentially, separately or in a single pharmaceutical formulation. Where the co-administered compounds or compositions are administered in separate dosage forms, the number of dosages administered per day for each compound may be the same or different. The compounds, conjugates, and/or compositions can be administered via the same or different routes of administration as each other and/or with a second therapy. The compounds, conjugates, or compositions can be administered according to simultaneous or alternating regimens, at the same or different times during the course of a therapy, concurrently in divided or single forms.

Combined with the teachings provided herein, by choosing among the various active compounds and weighing factors such as potency, relative bioavailability, patient body weight, severity of adverse side-effects and mode of administration, an effective prophylactic or therapeutic treatment regimen can be planned which does not cause substantial unwanted toxicity and yet is effective to treat the particular subject. The effective amount for any particular application can vary depending on such factors as the disease or condition being treated, the particular compound being administered, the size of the subject, or the severity of the disease or condition. One of ordinary skill in the art can empirically determine the effective amount of a particular compound and/or other therapeutic agent without necessitating undue experimentation.

For example, for any compound, effective amount can be initially determined from animal models. An effective dose can also be determined from human data for compounds which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

A maximum dose can be used, that is, the highest safe dose according to some medical judgment. Multiple doses per day can be used to achieve appropriate systemic levels of compounds. Appropriate systemic levels can be determined by, for example, measurement of the patient's peak or sustained plasma level of the drug.

“Dose” and “dosage” are used interchangeably herein.

Generally, daily oral doses of a compound are, for human subjects, from about 0.01 milligrams/kg per day to 1,000 milligrams/kg per day. Oral doses in the range of 0.5 to 50 milligrams/kg, in one or more administrations per day, can yield therapeutic results. Dosage can be adjusted appropriately to achieve desired drug levels, local or systemic, depending upon the mode of administration. For example, intravenous administration can vary from one order to several orders of magnitude lower dose per day. If the response in a subject is insufficient at such doses, even higher doses (or effective higher doses by a different, more localized delivery route) can be employed to the extent that patient tolerance permits. Multiple doses per day are contemplated to achieve appropriate systemic levels of the compound.

For clinical use, any compound or conjugate can be administered in an amount equal or equivalent to 0.2-2,000 milligram (mg) of compound per kilogram (kg) of body weight of the subject per day. The compounds can be administered in a dose equal or equivalent to 2-2,000 mg of compound per kg body weight of the subject per day. The compounds can be administered in a dose equal or equivalent to 20-2,000 mg of compound per kg body weight of the subject per day. The compounds can be administered in a dose equal or equivalent to 50-2,000 mg of compound per kg body weight of the subject per day. The compounds can be administered in a dose equal or equivalent to 100-2,000 mg of compound per kg body weight of the subject per day. The compounds can be administered in a dose equal or equivalent to 200-2,000 mg of compound per kg body weight of the subject per day. Where a precursor or prodrug of a compound is to be administered, it is administered in an amount that is equivalent to, i.e., sufficient to deliver, the above-stated amounts of the compound.

The formulations of the compounds or pharmaceutically acceptable salts thereof can be administered to human subjects in effective amounts. Typical dose ranges can be from about 0.01 microgram/kg to about 2 mg/kg of body weight per day. The dosage of drug to be administered is likely to depend on such variables as the type and extent of the disorder, the overall health status of the particular subject, the specific compound being administered, the excipients used to formulate the compound, and its route of administration. Routine experiments can be used to optimize the dose and dosing frequency for any particular compound or pharmaceutically acceptable salt thereof.

The compounds or pharmaceutically acceptable salts thereof can be administered at a concentration in the range from about 0.001 microgram/kg to greater than about 500 mg/kg. For example, the concentration can be 0.001 microgram/kg, 0.01 microgram/kg, 0.05 microgram/kg, 0.1 microgram/kg, 0.5 microgram/kg, 1.0 microgram/kg, 10.0 microgram/kg, 50.0 microgram/kg, 100.0 microgram/kg, 500 microgram/kg, 1.0 mg/kg, 5.0 mg/kg, 10.0 mg/kg, 15.0 mg/kg, 20.0 mg/kg, 25.0 mg/kg, 30.0 mg/kg, 35.0 mg/kg, 40.0 mg/kg, 45.0 mg/kg, 50.0 mg/kg, 60.0 mg/kg, 70.0 mg/kg, 80.0 mg/kg, 90.0 mg/kg, 100.0 mg/kg, 150.0 mg/kg, 200.0 mg/kg, 250.0 mg/kg, 300.0 mg/kg, 350.0 mg/kg, 400.0 mg/kg, 450.0 mg/kg, to greater than about 500.0 mg/kg or any incremental value thereof. It is to be understood that all values and ranges between these values and ranges are meant to be encompassed.

The compounds or pharmaceutically acceptable salts thereof can be administered at a dosage in the range from about 0.2 milligram/kg/day to greater than about 100 mg/kg/day. For example, the dosage can be 0.2 mg/kg/day to 100 mg/kg/day, 0.2 mg/kg/day to 50 mg/kg/day, 0.2 mg/kg/day to 25 mg/kg/day, 0.2 mg/kg/day to 10 mg/kg/day, 0.2 mg/kg/day to 7.5 mg/kg/day, 0.2 mg/kg/day to 5 mg/kg/day, 0.25 mg/kg/day to 100 mg/kg/day, 0.25 mg/kg/day to 50 mg/kg/day, 0.25 mg/kg/day to 25 mg/kg/day, 0.25 mg/kg/day to 10 mg/kg/day, 0.25 mg/kg/day to 7.5 mg/kg/day, 0.25 mg/kg/day to 5 mg/kg/day, 0.5 mg/kg/day to 50 mg/kg/day, 0.5 mg/kg/day to 25 mg/kg/day, 0.5 mg/kg/day to 20 mg/kg/day, 0.5 mg/kg/day to 15 mg/kg/day, 0.5 mg/kg/day to 10 mg/kg/day, 0.5 mg/kg/day to 7.5 mg/kg/day, 0.5 mg/kg/day to 5 mg/kg/day, 0.75 mg/kg/day to 50 mg/kg/day, 0.75 mg/kg/day to 25 mg/kg/day, 0.75 mg/kg/day to 20 mg/kg/day, 0.75 mg/kg/day to 15 mg/kg/day, 0.75 mg/kg/day to 10 mg/kg/day, 0.75 mg/kg/day to 7.5 mg/kg/day, 0.75 mg/kg/day to 5 mg/kg/day, 1.0 mg/kg/day to 50 mg/kg/day, 1.0 mg/kg/day to 25 mg/kg/day, 1.0 mg/kg/day to 20 mg/kg/day, 1.0 mg/kg/day to 15 mg/kg/day, 1.0 mg/kg/day to 10 mg/kg/day, 1.0 mg/kg/day to 7.5 mg/kg/day, 1.0 mg/kg/day to 5 mg/kg/day, 2 mg/kg/day to 50 mg/kg/day, 2 mg/kg/day to 25 mg/kg/day, 2 mg/kg/day to 20 mg/kg/day, 2 mg/kg/day to 15 mg/kg/day, 2 mg/kg/day to 10 mg/kg/day, 2 mg/kg/day to 7.5 mg/kg/day, or 2 mg/kg/day to 5 mg/kg/day.

The compounds can be administered at a dosage in the range from about 0.25 milligram/kg/day to about 25 mg/kg/day. For example, the dosage can be 0.25 mg/kg/day, 0.5 mg/kg/day, 0.75 mg/kg/day, 1.0 mg/kg/day, 1.25 mg/kg/day, 1.5 mg/kg/day, 1.75 mg/kg/day, 2.0 mg/kg/day, 2.25 mg/kg/day, 2.5 mg/kg/day, 2.75 mg/kg/day, 3.0 mg/kg/day, 3.25 mg/kg/day, 3.5 mg/kg/day, 3.75 mg/kg/day, 4.0 mg/kg/day, 4.25 mg/kg/day, 4.5 mg/kg/day, 4.75 mg/kg/day, 5 mg/kg/day, 5.5 mg/kg/day, 6.0 mg/kg/day, 6.5 mg/kg/day, 7.0 mg/kg/day, 7.5 mg/kg/day, 8.0 mg/kg/day, 8.5 mg/kg/day, 9.0 mg/kg/day, 9.5 mg/kg/day, 10 mg/kg/day, 11 mg/kg/day, 12 mg/kg/day, 13 mg/kg/day, 14 mg/kg/day, 15 mg/kg/day, 16 mg/kg/day, 17 mg/kg/day, 18 mg/kg/day, 19 mg/kg/day, 20 mg/kg/day, 21 mg/kg/day, 22 mg/kg/day, 23 mg/kg/day, 24 mg/kg/day, 25 mg/kg/day, 26 mg/kg/day, 27 mg/kg/day, 28 mg/kg/day, 29 mg/kg/day, 30 mg/kg/day, 31 mg/kg/day, 32 mg/kg/day, 33 mg/kg/day, 34 mg/kg/day, 35 mg/kg/day, 36 mg/kg/day, 37 mg/kg/day, 38 mg/kg/day, 39 mg/kg/day, 40 mg/kg/day, 41 mg/kg/day, 42 mg/kg/day, 43 mg/kg/day, 44 mg/kg/day, 45 mg/kg/day, 46 mg/kg/day, 47 mg/kg/day, 48 mg/kg/day, 49 mg/kg/day, or 50 mg/kg/day.

The compound, pharmaceutically acceptable salt thereof, or precursor thereof can be administered in concentrations that range from 0.01 micromolar to greater than or equal to 500 micromolar. For example, the dose can be 0.01 micromolar, 0.02 micromolar, 0.05 micromolar, 0.1 micromolar, 0.15 micromolar, 0.2 micromolar, 0.5 micromolar, 0.7 micromolar, 1.0 micromolar, 3.0 micromolar, 5.0 micromolar, 7.0 micromolar, 10.0 micromolar, 15.0 micromolar, 20.0 micromolar, 25.0 micromolar, 30.0 micromolar, 35.0 micromolar, 40.0 micromolar, 45.0 micromolar, 50.0 micromolar, 60.0 micromolar, 70.0 micromolar, 80.0 micromolar, 90.0 micromolar, 100.0 micromolar, 150.0 micromolar, 200.0 micromolar, 250.0 micromolar, 300.0 micromolar, 350.0 micromolar, 400.0 micromolar, 450.0 micromolar, to greater than about 500.0 micromolar or any incremental value thereof. It is to be understood that all values and ranges between these values and ranges are meant to be encompassed.

The compound, or pharmaceutically acceptable salt thereof, or precursor thereof can be administered at concentrations that range from 0.10 microgram/mL to 500.0 microgram/mL. For example, the concentration can be 0.10 microgram/mL, 0.50 microgram/mL, 1 microgram/mL, 2.0 microgram/mL, 5.0 microgram/mL, 10.0 microgram/mL, 20 microgram/mL, 25 microgram/mL. 30 microgram/mL, 35 microgram/mL, 40 microgram/mL, 45 microgram/mL, 50 microgram/mL, 60.0 microgram/mL, 70.0 microgram/mL, 80.0 microgram/mL, 90.0 microgram/mL, 100.0 microgram/mL, 150.0 microgram/mL, 200.0 microgram/mL, 250.0 g/mL, 250.0 micro gram/mL, 300.0 microgram/mL, 350.0 microgram/mL, 400.0 microgram/mL, 450.0 microgram/mL, to greater than about 500.0 microgram/mL or any incremental value thereof. It is to be understood that all values and ranges between these values and ranges are meant to be encompassed.

The formulations can be administered in pharmaceutically acceptable solutions, which can routinely contain pharmaceutically acceptable concentrations of salt, buffering agents, preservatives, compatible carriers, adjuvants, and optionally other therapeutic ingredients. For use in therapy, an effective amount of the compound can be administered to a subject by any mode that delivers the compound to the desired surface. Administering a pharmaceutical composition can be accomplished by any means known to the skilled artisan. Routes of administration include, but are not limited to, intravenous, intramuscular, intraperitoneal, intravesical (urinary bladder), oral, subcutaneous, direct injection (for example, into a tumor or abscess), mucosal (e.g., topical to eye), inhalation, and topical.

For intravenous and other parenteral routes of administration, a compound can be formulated as a lyophilized preparation, as a lyophilized preparation of liposome-intercalated or -encapsulated active compound, as a lipid complex in aqueous suspension, or as a salt complex. Lyophilized formulations are generally reconstituted in suitable aqueous solution, e.g., in sterile water or saline, shortly prior to administration.

For oral administration, the compounds can be formulated readily by combining the active compound(s) with pharmaceutically acceptable carriers well-known in the art. Such carriers enable the compounds to be formulated as tablets, pills, dragées, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragée cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinyl pyrrolidone (PVP). If desired, disintegrating agents can be added, such as the cross-linked PVP, agar, or alginic acid or a salt thereof such as sodium alginate. Optionally the oral formulations can also be formulated in saline or buffers, e.g., ethylenediaminetetraacetic acid (EDTA) for neutralizing internal acid conditions, or can be administered without any carriers.

Also contemplated are oral dosage forms of the compounds. The compounds can be chemically modified so that oral delivery of the derivative is efficacious. Generally, the chemical modification contemplated is the attachment of at least one moiety to the compound itself, where said moiety permits (a) inhibition of acid hydrolysis; and (b) uptake into the blood stream from the stomach or intestine. Also desired is the increase in overall stability of the compounds and increase in circulation time in the body. Examples of such moieties include polyethylene glycol, copolymers of ethylene glycol and propylene glycol, carboxymethyl cellulose, dextran, polyvinyl alcohol, PVP and polyproline. Abuchowski and Davis, “Soluble Polymer-Enzyme Adducts,” In: Enzymes as Drugs, Hocenberg and Roberts, eds., Wiley-Interscience, New York, N.Y., pp. 367-383 (1981); Newmark et al., J Appl Biochem 4:185-189 (1982). Other polymers that could be used are poly-1,3-dioxolane and poly-1,3,6-tioxocane. For pharmaceutical usage, as indicated above, polyethylene glycol moieties are suitable.

The location of release of a compound hereof or pharmaceutically acceptable salt thereof can be the stomach, the small intestine (e.g., the duodenum, the jejunum, or the ileum), or the large intestine. One skilled in the art has available formulations, which will not dissolve in the stomach, yet will release the material in the duodenum or elsewhere in the intestine. The release can avoid the deleterious effects of the stomach environment, either by protection of the compound or by release of the compound or a pharmaceutically acceptable salt thereof beyond the stomach environment, such as in the intestine.

To ensure full gastric resistance a coating impermeable to at least pH 5.0 can be essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and shellac. These coatings can be used as mixed films.

A coating or mixture of coatings can also be used on tablets, which are not intended for protection against the stomach. This can include sugar coatings, or coatings which make the tablet easier to swallow. Capsules can consist of a hard shell (such as gelatin) for delivery of dry therapeutic (e.g., powder); for liquid forms, a soft gelatin shell can be used. The shell material of cachets could be thick starch or other edible paper. For pills, lozenges, molded tablets or tablet triturates, moist massing techniques can be used.

As noted above, the compound or pharmaceutically acceptable salt thereof can be included in the formulation as fine multi-particulates in the form of granules or pellets of particle size about 1 mm. The formulation of the material for capsule administration could also be as a powder, lightly compressed plugs or even as tablets.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. Microspheres formulated for oral administration can also be used. Such microspheres have been well defined in the art. All formulations for oral administration should be in dosages suitable for such administration.

For buccal administration, the compositions can take the form of tablets or lozenges formulated in conventional manner.

For topical administration, the compound can be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

For administration by inhalation, compounds can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g., gelatin for use in an inhaler or insufflator can be formulated containing a powder mix of the compound and a suitable powder base such as lactose or starch.

Also contemplated is pulmonary delivery of the compounds (or salts thereof). The compound is delivered to the lungs of a mammal while inhaling and traverses across the lung epithelial lining to the blood stream. Other reports of inhaled molecules include Adjei et al., Pharm Res 7:565-569 (1990); Adjei et al., Int J Pharmaceutics 63:135-144 (1990) (leuprolide acetate); Braquet et al., J Cardiovasc Pharmacol 13 (suppl. 5): 143-146 (1989) (endothelin-1); Hubbard et al., Annal Int Med 3:206-212 (1989) (al-antitrypsin); Smith et al., 1989, J Clin Invest 84:1145-1146 (a-1-proteinase); Oswein et al., 1990, “Aerosolization of Proteins,” Proceedings of Symposium on Respiratory Drug Delivery II, Keystone, Colorado, March, (recombinant hepatocyte growth hormone); Debs et al., 1988, J Immunol 140:3482-3488 (interferon-gamma and tumor necrosis factor alpha) and Platz et al., U.S. Pat. No. 5,284,656 (granulocyte colony stimulating factor; incorporated herein by reference). A method and composition for pulmonary delivery of drugs for systemic effect is described in U.S. Pat. No. 5,451,569 (specifically incorporated herein by reference for its disclosure regarding same), issued Sep. 19, 1995, to Wong et al.

Contemplated for use are a wide range of mechanical devices designed for pulmonary delivery of therapeutic products, including but not limited to nebulizers, metered dose inhalers, and powder inhalers, all of which are familiar to those skilled in the art.

Nasal delivery of a pharmaceutical composition is also contemplated. Nasal delivery allows the passage of a pharmaceutical composition to the blood stream directly after administering therapeutic product to the nose, without the necessity for deposition of the product in the lung. Formulations for nasal delivery include those with dextran or cyclodextran.

The compounds, when it is desirable to deliver them systemically, can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

Pharmaceutical formulations for parenteral administration include aqueous solutions of the active compounds in water-soluble form. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethylcellulose, sorbitol, or dextran. Optionally, the suspension can also contain suitable stabilizers or agents which increase the solubility of the compounds to allow for the preparation of highly concentrated solutions.

Alternatively, the active compounds can be in powder form for constitution with a suitable vehicle, e.g., sterile pyrogen-free water, before use.

The compounds can also be formulated in rectal or vaginal compositions such as suppositories or retention enemas, e.g., containing conventional suppository bases such as cocoa butter or other glycerides.

In addition to the formulations described above, a compound can also be formulated as a depot preparation. Such long-acting formulations can be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also can comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro) capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer R, Science 249:1527-1533 (1990).

Disintegrants can be included in the formulation of therapeutic agent into a solid dosage form. Materials used as disintegrates include, but are not limited to, starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrant is the insoluble cationic exchange resin. Powdered gums can be used as disintegrants and as binders and these can include powdered gums such as agar, Karaya or tragacanth. Alginic acid and its sodium salt are also useful as disintegrants.

Binders can be used to hold the compound together to form a hard tablet and include materials from natural products such as acacia, tragacanth, starch and gelatin. Others include methyl cellulose (MC), ethyl cellulose (EC) and carboxymethyl cellulose (CMC). PVP and hydroxypropylmethyl cellulose (HPMC) can both be used in alcoholic solutions to granulate therapeutic agent.

An anti-frictional agent can be included in the formulation of therapeutic to prevent sticking during the formulation process. Lubricants can be used as a layer between therapeutic agent and the die wall, and these can include, but are not limited to, stearic acid, including its magnesium and calcium salts, polytetrafluoroethylene (PTFE), liquid paraffin, vegetable oils and waxes. Soluble lubricants can also be used, such as sodium lauryl sulfate, magnesium lauryl sulfate, polyethylene glycol of various molecular weights, Carbowax 4000 and 6000.

Glidants, which can improve the flow properties of the drug during formulation and aid rearrangement during compression, can be added. The glidants can include starch, talc, pyrogenic silica and hydrated silicoaluminate.

To aid dissolution of therapeutic agent into the aqueous environment a surfactant can be added as a wetting agent. Surfactants can include anionic detergents, such as sodium lauryl sulfate, dioctyl sodium sulfosuccinate and dioctyl sodium sulfonate. Cationic detergents which can be used include benzalkonium chloride and benzethonium chloride. Potential non-ionic detergents that can be included in the formulation as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the compound or derivative thereof either alone or as a mixture in different ratios.

The compound can be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

One can dilute or increase the volume of the compound with an inert material. These diluents can include carbohydrates, especially mannitol, a-lactose, anhydrous lactose, cellulose, sucrose, modified dextrans and starch. Certain inorganic salts also can be used as fillers including calcium triphosphate, magnesium carbonate and sodium chloride. Some commercially available diluents are Fast-Flo, Emdex, STA-Rx 1500, Emcompress and Avicell.

Suitable buffering agents include acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Pharmaceutical compositions contain an effective amount of a compound as described herein and optionally one or more other therapeutic agents included in a pharmaceutically acceptable carrier. The term “pharmaceutically acceptable carrier” means one or more compatible solid or liquid fillers, diluents or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also can be commingled with the compounds, and with each other, in a manner such that there is no interaction which would substantially impair the desired pharmaceutical efficiency.

Therapeutic agent(s), including specifically, but not limited to, a compound hereof, can be provided in particles. “Particles” as used herein means nanoparticles or microparticles (or in some instances larger particles) that can consist in whole or in part of the compound or the other therapeutic agent(s) as described herein. The particles can contain therapeutic agent(s) in a core surrounded by a coating, including, but not limited to, an enteric coating. Therapeutic agent(s) also can be dispersed throughout the particles. Therapeutic agent(s) also can be adsorbed into the particles. The particles can be of any order release kinetics, including zero-order release, first-order release, second-order release, delayed release, sustained release, immediate release, and any combination thereof, etc. The particle can include, in addition to therapeutic agent(s), any of those materials routinely used in the art of pharmacy and medicine, including, but not limited to, erodible, nonerodible, biodegradable, or nonbiodegradable material or combinations thereof. The particles can be microcapsules which contain the compound in a solution or in a semi-solid state. The particles can be of virtually any shape.

Both non-biodegradable and biodegradable polymeric materials can be used in the manufacture of particles for delivering therapeutic agent(s). Such polymers can be natural or synthetic polymers. The polymer is selected based on the period of time over which release is desired. Bioadhesive polymers of particular interest include bioerodible hydrogels described in Sawhney et al., Macromolecules 26:581-587 (1993), the teachings of which are specifically incorporated by reference herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The compound can be contained in controlled-release systems. The term “controlled release” is intended to refer to any drug-containing formulation in which the manner and profile of drug release from the formulation are controlled. This refers to immediate as well as non-immediate release formulations, with non-immediate release formulations including, but not limited to, sustained release and delayed release formulations. The term “sustained release” (also referred to as “extended release”) is used in its conventional sense to refer to a drug formulation that provides for gradual release of a drug over an extended period of time, and that can result in substantially constant blood levels of a drug over an extended time period. The term “delayed release” is used in its conventional sense to refer to a drug formulation in which there is a time delay between administration of the formulation and the release of the drug therefrom. “Delayed release” may or may not involve gradual release of drug over an extended period of time, and thus may or may not be “sustained release.”

Use of a long-term sustained release implant can be particularly suitable for treatment of chronic conditions. “Long-term” release, as used herein, means that the implant is constructed and arranged to deliver therapeutic levels of the active ingredient for at least 7 days, and up to 30-60 days. Long-term sustained release implants are well-known to those of ordinary skill in the art and include some of the release systems described above.

Depending upon the route of administration, a wide range of permissible dosages are contemplated herein, including doses falling in the range from about 10⁶ to 10¹¹ virus particles (VP)/kg. The dosages may be single or divided and may be administered according to a wide variety of protocols, including q.d. (once a day), b.i.d. (twice a day), t.i.d. (three times a day), or even every other day, once a week, once a month, once a quarter, and the like. In each of these cases it is understood that the effective amounts described herein correspond to the instance of administration, or alternatively to the total daily, weekly, month, or quarterly dose, as determined by the dosing protocol.

In addition to the illustrative dosages and dosing protocols described herein, an effective amount of any one or a mixture of the compounds described herein can be determined by the attending diagnostician or physician by the use of known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved, the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

In certain embodiments, a use of a compound, conjugate, or pharmaceutically acceptable salt of the compound or conjugate in the manufacture of a medicament for the treatment of a disease in a subject is provided. The compound or conjugate can be any compound or conjugate hereof. The disease in the subject can be a disease where modulation of a kinase reduces the severity of or otherwise treats such disease in the subject. The disease can be any disease state described herein. In certain embodiments, the disease is cancer, diabetes, an inflammatory disease, or a neurological disease.

Any of the compounds and/or conjugates hereof can be for use in the treatment of a disease state modulated by one or more kinases. The disease state can be a cancer and the one or more kinases can be CKD, FLT3, and/or haspin kinase.

Those skilled in the art will recognize that numerous modifications can be made to the specific implementations described above. The implementations should not be limited to the particular limitations described. Other implementations may be possible.

While the compounds, conjugates, and pharmaceutical compositions have been illustrated and described in detail in the foregoing description, the same is to be considered as illustrative and not restrictive in character, it being understood that only certain embodiments have been shown and described and that all changes and modifications that come within the spirit of the invention are desired to be protected.

It is intended that that the scope of the present compounds, compositions, and methods are defined by the following claims. However, this disclosure may be practiced otherwise than is specifically explained and illustrated without departing from its spirit or scope. Those skilled in the art will understand that various alternatives to the embodiments described herein can be employed in practicing the claims without departing from the spirit and scope as defined in the following claims.

Any use of section headings is intended to aid reading of the document and is not to be interpreted as limiting. Further, information that is relevant to a section heading may occur within or outside of that particular section.

All publications, patents, patent application publications, journal articles, textbooks, and other publications referred to in this document are indicative of the level of skill of those in the art to which the disclosure pertains. All such publications are incorporated herein by reference to the same extent as if each individual publication were specifically and individually indicated to be incorporated by reference. In the event of inconsistent usages between this document and those documents so incorporated by reference, the usage in the incorporated reference should be considered supplementary to that of this document; for irreconcilable inconsistencies, the usage in this document controls.

Various techniques and mechanisms will sometimes describe a connection or link between two components. Words such as attached, linked, coupled, connected, and similar terms with their inflectional morphemes are used interchangeably, unless the difference is noted or made otherwise clear from the context. These words and expressions do not necessarily signify direct connections but include connections through mediate components. It should be noted that a connection between two components does not necessarily mean a direct, unimpeded connection, as a variety of other components may reside between the two components of note. Consequently, a connection does not necessarily mean a direct, unimpeded connection unless otherwise noted.

### Certain Definitions

As used herein, the following terms and phrases shall have the meanings set forth below. Unless defined otherwise, all technical and scientific terms used herein have the same meaning as commonly understood to one of ordinary skill in the art.

The term “about” allows for a degree of variability in a value or range, for example, within 10%, within 5%, or within 1% of a stated value or of a stated limit of a range. In the present disclosure the term “substantially” can allow for a degree of variability in a value or range, for example, within 90%, within 95%, 99%, 99.5%, 99.9%, 99.99%, or at least about 99.999% or more of a stated value or of a stated limit of a range.

The terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation.

The term “or” is used to refer to a nonexclusive “or” unless otherwise indicated. In addition, it is to be understood that the phraseology or terminology employed herein, and not otherwise defined, is for the purpose of description only and not of limitation.

The term “substituted” as used herein refers to a functional group in which one or more hydrogen atoms contained therein are replaced by one or more non-hydrogen atoms. The term “functional group” or “substituent” as used herein refers to a group that can be or is substituted onto a molecule. Examples of substituents or functional groups include, but are not limited to, a halogen (e.g., F, Cl, Br, and I); an oxygen atom in groups such as hydroxyl groups, alkoxy groups, aryloxy groups, aralkyloxy groups, oxo (carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, azides, hydroxylamines, cyano, nitro groups, N-oxides, hydrazides, and enamines; and other heteroatoms in various other groups.

The term “alkyl” as used herein refers to substituted or unsubstituted straight chain and branched alkyl groups and cycloalkyl groups having from 1 to about 20 carbon atoms (C₁-C₂₀), 1 to 12 carbons (C₁-C₁₂), 1 to 8 carbon atoms (C₁-C₈), or, in some embodiments, from 1 to 6 carbon atoms (C₁-C₆). Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to substituted or unsubstituted straight chain and branched divalent alkenyl and cycloalkenyl groups having from 2 to 20 carbon atoms (C₂-C₂₀), 2 to 12 carbons (C₂-C₁₂), 2 to 8 carbon atoms (C₂-C₈) or, in some embodiments, from 2 to 4 carbon atoms (C₂-C₄) and at least one carbon-carbon double bond. Examples of straight chain alkenyl groups include those with from 2 to 8 carbon atoms such as —CH—CH—, —CH═CHCH₂—, and the like. Examples of branched alkenyl groups include, but are not limited to, —CH═C(CH₃)— and the like.

An alkynyl group is the fragment, containing an open point of attachment on a carbon atom that would form if a hydrogen atom bonded to a triply bonded carbon is removed from the molecule of an alkyne. The term “hydroxyalkyl” as used herein refers to alkyl groups as defined herein substituted with at least one hydroxyl (—OH) group.

The term “cycloalkyl” as used herein refers to substituted or unsubstituted cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. In some embodiments, cycloalkyl groups can have 3 to 6 carbon atoms (C₃-C₆). Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like.

The term “acyl” as used herein refers to a group containing a carbonyl moiety wherein the group is bonded via the carbonyl carbon atom. The carbonyl carbon atom is also bonded to another carbon atom, which can be part of a substituted or unsubstituted alkyl, aryl, aralkyl cycloalkyl, cycloalkylalkyl, heterocyclyl, heterocyclylalkyl, heteroaryl, heteroarylalkyl group or the like. In the special case wherein the carbonyl carbon atom is bonded to a hydrogen, the group is a “formyl” group, an acyl group as the term is defined herein. An acyl group can include 0 to about 12-40, 6-10, 1-5 or 2-5 additional carbon atoms bonded to the carbonyl group. An acryloyl group is an example of an acyl group. An acyl group can also include heteroatoms within the meaning here. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “aryl” as used herein refers to substituted or unsubstituted cyclic aromatic hydrocarbons that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons (C₆-C₁₄) or from 6 to 10 carbon atoms (C₆-C₁₀) in the ring portions of the groups. Aryl groups can be unsubstituted or substituted, as defined herein. Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed herein.

The term “aralkyl” and “arylalkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “heterocyclyl” as used herein refers to substituted or unsubstituted aromatic and non-aromatic ring compounds containing 3 or more ring members, of which, one or more is a heteroatom such as, but not limited to, B, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. In some embodiments, heterocyclyl groups include heterocyclyl groups that include 3 to 8 carbon atoms (C₃-C₈), 3 to 6 carbon atoms (C₃-C₆) or 6 to 8 carbon atoms (C₆-C₈).

A heteroaryl ring is an embodiment of a heterocyclyl group. The phrase “heterocyclyl group” includes fused ring species including those that include fused aromatic and non-aromatic groups. Representative heterocyclyl groups include, but are not limited to pyrrolidinyl, azetidinyl, piperidynyl, piperazinyl, morpholinyl, chromanyl, indolinonyl, isoindolinonyl, furanyl, pyrrolidinyl, pyridinyl, pyrazinyl, pyrimidinyl, triazinyl, thiophenyl, tetrahydrofuranyl, pyrrolyl, oxazolyl, oxadiazolyl, imidazolyl, triazyolyl, tetrazolyl, benzoxazolinyl, benzthiazolinyl, and benzimidazolinyl groups.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclylalkyl groups include, but are not limited to, furan-2-yl methyl, furan-3-yl methyl, pyridine-3-yl methyl, tetrahydrofuran-2-yl methyl, and indol-2-yl propyl.

The term “heteroarylalkyl” as used herein refers to alkyl groups as defined herein in which a hydrogen or carbon bond of an alkyl group is replaced with a bond to a heteroaryl group as defined herein.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can further include double or triple bonds, and can also include heteroatoms. For example, an allyloxy group is an alkoxy group within the meaning herein. A methoxyethoxy group is also an alkoxy group within the meaning herein, as is a methylenedioxy group in a context where two adjacent atoms of a structure are substituted therewith.

The term “amine” as used herein refers to primary, secondary, and tertiary amines having, e.g., the formula N (group) 3 wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃+, wherein each R is independently selected, and protonated forms of each, except for —NR₃+, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, —CF(CH₃) 2 and the like.

The term “optionally substituted,” or “optional substituents,” as used herein, means that the groups in question are either unsubstituted or substituted with one or more of the substituents specified. When the groups in question are substituted with more than one substituent, the substituents may be the same or different. When using the terms “independently,” “independently are,” and “independently selected from” mean that the groups in question may be the same or different. Certain of the herein defined terms may occur more than once in the structure, and upon such occurrence each term shall be defined independently of the other.

The terms “patient” and “subject” are used interchangeably and include a human patient, a laboratory animal, such as a rodent (e.g., mouse, rat, or hamster), a rabbit, a monkey, a chimpanzee, a domestic animal, such as a dog, a cat, or a rabbit, an agricultural animal, such as a cow, a horse, a pig, a sheep, a goat, an livestock animal raised for food production, or a wild animal in captivity, such as a bear, a panda, a lion, a tiger, a leopard, an elephant, a zebra, a giraffe, a gorilla, a dolphin, or a whale. The patient to be treated is preferably a mammal, in particular a human being.

The terms “protein,” “polypeptide” and “peptide” refer to compounds comprising amino acids joined via peptide bonds and are used interchangeably.

### EXAMPLES

The following examples serve to illustrate the present disclosure. The examples are not intended to limit the scope of the claims in any way.

### Example 1

### Synthesis of CDK Inhibitor Compounds

Unless noted otherwise, all reagents and solvents were purchased from commercial sources and used as received. All reactions were performed in a screw-cap sealed vials unless otherwise stated. The 1H and ¹³C NMR spectra were obtained in (CD₃)₂SO as solvent using a 500 MHz or 800 MHz spectrometer with Me4Si as an internal standard. Chemical shifts were reported in parts per million (8) and are calibrated using residual un-deuterated solvent as an internal reference.

Data for ¹H NMR spectra are reported as follows: chemical shift (8 ppm) (multiplicity, coupling constant (Hz), integration). Multiplicities are reported as follows: s=singlet, d=doublet, t=triplet, q=quartet, m=multiplet, or combinations thereof.

Method A, generally

In a 250 mL round bottom flask equipped with a stir bar 5-aminoindazole (10 mmol) and the appropriate aldehyde, (4-formylbenzoic acid or 4-formylbenzonitrile) (10 mmol) was dissolved in 150 mL of 200 proof ethanol. This mixture was refluxed at 120° C. for 2 hours, after which the appropriate ketone (20 mmol) and a catalytic amount of hydrochloric acid was added. This was allowed to stir at reflux for an additional 12 hours. The reaction was concentrated and purified at 40% ethyl acetate and 60% hexanes.

Method B, generally

In a 50 mL round bottom flask equipped with a stir bar was added 4-(8,9,10,11-tetrahydro-3H-pyrazolo[4,3-a]phenanthridin-7-yl)benzonitrile (850 mg, 2.53 mmol) and NaOH (532 mg, 13 mmol) was dissolved in ethanol (4 mL) and water (8 mL). This was added to the round bottom flask and allowed to reflux for 12 hours, after about an hour the mixture completely dissolved. After completion of the reaction, the solvent was evaporated under reduced pressure and diluted with water and acidified to pH 5 using HCl. This is then stirred for 30 minutes to assure the pH was stable. The product is subsequently collected via vacuum filtration.

Method C, generally

In a 20 mL vial equipped with a stir bar the acid (0.5 mmol) was dissolved in N,N-dimethylformamide (DMF) (2 mL) and N,N-diisopropylethylamine (DIPEA) (12.5 eq). Bis(2-oxo-3-oxazolidinyl)phosphinic chloride (2.7 eq) was added to this solution and allowed to stir at 50° C. for 30 minutes, after which the corresponding amine (0.5 mmol) was added and stirred at 50° C. overnight. The DMF was concentrated under reduced pressure and subsequently purified with 15% methanol and 85% ethyl acetate.

Method D, generally

In a 20 mL vial equipped with a stir bar, the acid (0.5 mmol) and 4-dimethylaminopyridine (12 mg), N-methylmorpholine (1 mmol), and (1-[Bis(dimethylamino)methylene]-1H-1,2,3-triazolo[4,5-b]pyridinium 3-oxide hexafluorophosphate, Hexafluorophosphate Azabenzotriazole Tetramethyl Uronium) was dissolved in DMF (2 mL) and allowed to stir at room temperature. After 30 minutes, 1-methylpiperazine (2 mmol) was added and stirred at room temperature overnight. DMF was concentrated under reduced pressure and the reaction was purified with 25% methanol and 75% ethyl acetate.

Method E, generally

In a microwave vial, the acid (0.5 mmol), Fluoro-N,N,N′,N′-bis(tetramethylene) formamidinium hexafluorophosphate (0.6 mmol), DIPEA (1.75 mmol) and dichloromethane (2 mL) were stirred at room temperature for 30 minutes. Afterwards, the amine (0.38 mmol) was added and stirred in a sand bath at 80° C. overnight. The reaction mixture was concentrated and purified with 10% methanol and 90% ethyl acetate.

Method F, generally

In a 20 mL vial equipped with a stir bar, the acid intermediate (0.5 mmol) and Hexafluorophosphate Azabenzotriazole Tetramethyl Uronium) were sealed with a septum, air was vacuumed and a N₂ balloon was added. To this reaction vial, anhydrous DMF (5 mL) and DIPEA (1.2 mL) were added. This was heated at 100° C. for 10 mins and then the amine (1 mmol) was added to reaction and allowed to stir at 100° C. overnight. The reaction mixtures were concentrated and purified via silica gel chromatography.

Method G, generally (synthesis of Compound HSH3165)

In a 25 mL round bottom flask equipped with a stir bar, N-(2-aminoethyl)-4-(8,9,10,11-tetrahydro-3H-pyrazolo[4,3-a]phenanthridin-7-yl)benzamide was dissolved in trifluoroacetic acid (2 mL) and dichloromethane (1 mL) and stirred overnight at room temperature. The reaction was concentrated to give pure product Compound HSH3165.

Method H, generally

To a solution of Compound HSH3107 (0.5 mmol) in anhydrous tetrahydrofuran (THF) (720 μL) was added lithium aluminum hydride (1.5 mmol, 1M in THF) at 0° C. The mixture was heated to 50° C. for two hours. This was cooled to 0° C. and worked up with water and thereafter stirred at room temperature for 3 minutes.

Solid was filtered through a bed of celite, washed with THE, dried with sodium sulfate, concentrated under reduced pressure and purified via silica gel column chromatography with [hexanes: ethyl acetate (30:70 to 0:100)].

### Intermediates:

### Synthesis of 4-(8,9,10,11-tetrahydro-3H-8,11-methanopyrazolo[4,3-a]phenanthridin-7-yl)benzoic acid

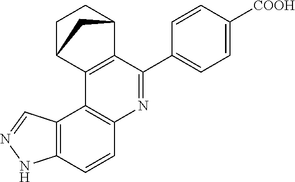 [SMILES: O=C(O)c1ccc(-c2nc3ccc4[nH]ncc4c3c3c2[C@H]2CC[C@@H]3C2)cc1]

In a 250 mL round bottom flask equipped with a stir bar 5-aminoindazole (2 mmol) and 4-formylbenzoic acid (2 mmol) was dissolved in 10 mL of 200 proof ethanol. This mixture was refluxed at 120° C. for 2 hours, after which bicyclo[2.2.1]heptan-2-one (4 mmol) and a catalytic amount of hydrochloric acid was added. This was allowed to stir at reflux for an additional 12 hours. The precipitate was washed with ethanol and taken to the next step without further purification.

¹H NMR (800 MHZ, (CD₃)₂SO) δ 8.75 (s, 1H), 8.12 (dd, J=20.4, 7.8 Hz, 2H), 7.97 (d, J=7.9 Hz, 2H), 7.86 (q, J=9.3 Hz, 2H), 2.95 (s, 1H), 2.18 (dd, J=27.2, 13.6 Hz, 2H), 1.83 (d, J=8.9 Hz, 1H), 1.67 (d, J=9.0 Hz, 1H), 1.60 (s, 1H), 1.35 (d, J=10.6 Hz, 1H), 1.22 (t, J=9.9 Hz, 1H). ¹³C NMR (201 MHz, (CD₃)₂SO) δ 167.64, 152.79, 147.96, 145.12, 144.02, 139.74, 130.72, 130.16, 129.86, 129.12, 128.07, 127.20, 121.04, 116.74, 115.88, 49.88, 43.24, 42.39, 26.88, 25.31. HRMS (ESI) m/z calculated for C₂₂H₁₈N₃O₂[M+H]⁺356.1393, found 356.1392.

### 4-(8,9,10,11-tetrahydro-3H-pyrazolo[4,3-a]phenanthridin-7-yl)benzonitrile

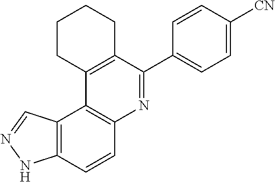 [SMILES: N#Cc1ccc(-c2nc3ccc4[nH]ncc4c3c3c2CCCC3)cc1]

Synthesized using Method A: Off white solid (1.5 g, 10 mmol, 46.3%). ¹H NMR (500 MHz, (CD₃)₂SO) δ 8.55 (s, 1H), 7.94-7.90 (m, 2H), 7.88-7.80 (m, 2H), 7.78-7.74 (m, 2H), 3.33 (s, 2H), 2.75 (t, J=6.1 Hz, 2H), 2.04-1.97 (m, 2H), 1.79-1.72 (m, 2H).¹³C NMR (126 MHz, (CD₃)₂SO) δ 155.09, 146.02, 143.76, 142.80, 138.90, 136.43, 132.38, 130.59, 129.59, 129.18, 122.28, 119.23, 116.43, 114.66, 111.07, 29.64, 28.64, 22.51, 22.43. HRMS (ESI) m/z calculated for C₂₁H₁₇N₄ [M+H]⁺325.1444, found 325.1448.

### 4-(9-methyl-3H-pyrazolo[4,3-f]quinolin-7-yl)benzonitrile

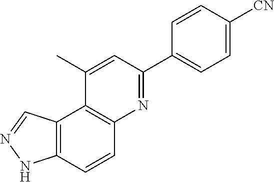 [SMILES: Cc1cc(-c2ccc(C#N)cc2)nc2ccc3[nH]ncc3c12]

Synthesized using Method A: Off white solid (0.840 g, 10 mmol, 30%). ¹H NMR (500 MHz, (CD₃)₂SO) δ 8.61-8.53 (m, 1H), 8.44-8.36 (m, 2H), 8.13 (s, 1H), 7.97-7.88 (m, 4H), 2.93 (s, 3H). ¹³C NMR (126 MHZ, (CD₃)₂SO) δ 151.30, 146.36, 144.74, 143.57, 138.94, 136.03, 133.06, 129.73, 127.94, 122.68, 120.92, 119.24, 116.71, 115.79, 111.79, 22.71. HRMS (ESI) m/z calculated for C₁₈H₁₃N₄ [M+H]⁺285.1135, found 285.1136.

### 4-(9-cyclopropyl-3H-pyrazolo[4,3-f]quinolin-7-yl)benzonitrile

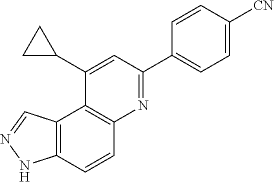 [SMILES: N#Cc1ccc(-c2cc(C3CC3)c3c(ccc4[nH]ncc43)n2)cc1]

Synthesized using Method A: Off white solid (0.485 g, 10 mmol, 14.7%). ¹H NMR (500 MHz, (CD₃)₂SO) δ 8.80 (s, 1H), 8.40 (d, J=8.3 Hz, 2H), 8.00-7.86 (m, 5H), 2.62 (m, 1H), 1.39-1.24 (m, 2H), 1.00 (q, J=5.3, 4.8 Hz, 2H). ¹³C NMR (126 MHZ, (CD₃)₂SO) δ 151.73, 149.26, 146.24, 143.69, 138.93, 136.66, 133.04, 129.80, 128.16, 123.61, 119.30, 117.24, 116.48, 116.06, 111.73, 40.40, 40.24, 40.06, 39.90, 39.73, 39.57, 39.40, 16.56, 7.80. HRMS (ESI) m/z calculated for C₂₀H₁₅N₄ [M+H]⁺311.1291, found 311.1293.

### 4-(1-methyl-8,9,10,11-tetrahydro-3H-pyrazolo[4,3-a]phenanthridin-7-yl)benzonitrile

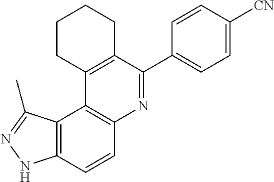 [SMILES: Cc1n[nH]c2ccc3nc(-c4ccc(C#N)cc4)c4c(c3c12)CCCC4]

Synthesized using Method A: Off white solid (402 mg, 2 mmol, 59.4%). ¹H NMR (500 MHz, (CD₃)₂SO) δ 7.88 (d, J=8.1 Hz, 2H), 7.71 (d, J=7.9 Hz, 4H), 3.37 (s, 2H), 2.83 (s, 3H), 2.74-2.65 (m, 2H), 1.75 (m, 4H). ¹³C NMR (126 MHZ, (CD₃)₂SO) δ 154.5, 146.0, 142.7, 133.1, 132.4, 130.7, 130.5, 129.8, 128.2, 127.9, 124.2, 124.1, 119.2, 114.9, 111.0, 49.1, 31.5, 27.8, 22.4, 22.1. HRMS (ESI) m/z calculated for C₁₈H₁₄N₃O₂ [M+H]⁺339.1604, found 339.1605.

### 3-fluoro-4-(8,9,10,11-tetrahydro-3H-pyrazolo[4,3-a]phenanthridin-7-yl)benzonitrile

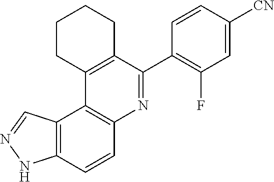 [SMILES: N#Cc1ccc(-c2nc3ccc4[nH]ncc4c3c3c2CCCC3)c(F)c1]

Synthesized using Method A: Off white solid (1.42 g, 10 mmol, 41.5%). ¹H NMR (500 MHz, (CD₃)₂SO) δ 8.56 (s, 1H), 7.94 (dd, J=9.5, 1.7 Hz, 1H), 7.88 (d, J=9.1 Hz, 1H), 7.85-7.78 (m, 2H), 7.68 (t, J=7.5 Hz, 1H), 3.36 (t, J=6.6 Hz, 2H), 2.60 (t, J=6.2 Hz, 2H), 2.06-1.92 (m, 2H), 1.85-1.71 (m, 2H). ¹³C NMR (126 MHZ, (CD₃)₂SO) δ 160.31 (d, 1J=248 Hz), 150.62, 143.80, 142.73, 138.94, 136.47, 134.29 (d, 2J=16 Hz), 133.27, 129.92, 129.44, 129.26, 122.57, 120.29 (2J-26 Hz), 118.00, 116.40, 114.82, 113.18 (d, 3J=10 Hz), 29.47, 27.26, 22.51, 22.09. HRMS (ESI) m/z calculated for C₂₁H₁₆N₄ [M+H]⁺343.1353, found 343.1352.

### 2-fluoro-4-(8,9,10,11-tetrahydro-3H-pyrazolo[4,3-a]phenanthridin-7-yl)benzonitrile

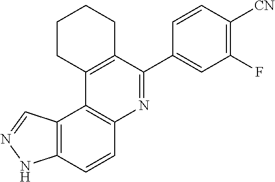 [SMILES: N#Cc1ccc(-c2nc3ccc4[nH]ncc4c3c3c2CCCC3)cc1F]

Synthesized using Method A: Off white solid (0.220 g, 5 mmol, 12.9%). ¹H NMR (500 MHZ, (CD₃)₂SO) δ 8.59 (s, 1H), 8.00 (t, J=7.4 Hz, 1H), 7.91-7.77 (m, 2H), 7.73 (d, J=10.4 Hz, 1H), 7.63 (d, J=8.0 Hz, 1H), 3.35 (s, 2H), 2.80 (t, J=6.2 Hz, 2H), 2.09-1.97 (m, 2H), 1.82-1.73 (m, 2H). ¹³C NMR (126 MHZ, (CD₃)₂SO) δ 163.68 (d, 1J=255 Hz), 153.84, 149.06, 149.00, 143.76, 143.02, 136.44, 133.99, 129.56, 129.22, 126.85, 122.55, 117.69, 117.53, 114.91, 114.46, 99.94 (d, 2J=15 Hz), 29.66, 28.46, 22.48, 22.40. HRMS (ESI) m/z calculated for C₂₁H₁₆N₄ [M+H]⁺343.1353, found 343.1352.

### 3-(8,9,10,11-tetrahydro-3H-pyrazolo[4,3-a]phenanthridin-7-yl)benzonitrile

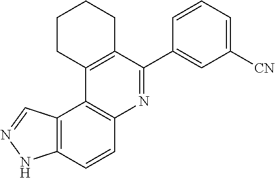 [SMILES: N#Cc1cccc(-c2nc3ccc4[nH]ncc4c3c3c2CCCC3)c1]

Synthesized using Method A: Off white solid (0.665 g, 10 mmol, 20.2%). ¹H NMR (500 MHz, (CD₃)₂SO) δ 8.54 (s, 1H), 7.98 (s, 1H), 7.91-7.82 (m, 3H), 7.80 (d, J=9.1 Hz, 1H), 7.67 (t, J=7.8 Hz, 1H), 2.74 (t, J=6.2 Hz, 2H), 2.01-1.91 (m, 2H), 1.73 (m, 2H). ¹³C NMR (126 MHz, (CD₃)₂SO) δ 154.67, 143.86, 142.83, 142.51, 134.35, 132.95, 131.92, 129.75, 129.55, 129.22, 127.67, 122.31, 119.13, 116.16, 115.89, 115.09, 111.75, 29.62, 28.62, 22.47, 22.42. HRMS (ESI) m/z calculated for C₂₁H₁₇N₄ [M+H]⁺325.1448, found 325.1449.

### 3-methoxy-4-(8,9,10,11-tetrahydro-3H-naphtho[1,2-e]indazol-7-yl)benzonitrile

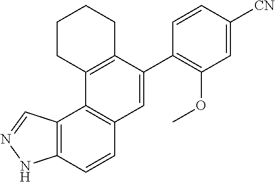 [SMILES: COc1cc(C#N)ccc1-c1cc2ccc3[nH]ncc3c2c2c1CCCC2]

Synthesized using Method A: Off white solid (0.255 g, 10 mmol, 7.21%). ¹H NMR (500 MHz, (CD₃)₂SO) δ 8.55 (s, 1H), 7.85 (d, J=9.0 Hz, 1H), 7.80 (s, 1H), 7.59 (s, 1H), 7.51 (dd, J=7.6, 1.4 Hz, 1H), 7.41 (d, J=7.7 Hz, 1H), 3.78 (s, 3H), 3.34 (d, J=16.9 Hz, 2H), 2.59 (s, 1H), 2.42 (d, J=16.5 Hz, 1H), 1.98 (dd, J=13.7, 6.5 Hz, 2H), 1.84-1.68 (m, 2H). ¹³C NMR (126 MHz, (CD₃)₂SO) δ 157.37, 153.80, 143.74, 143.68, 141.82, 136.31, 135.78, 131.79, 130.19, 129.49, 125.20, 119.17, 116.47, 115.27, 114.35, 114.31, 112.47, 56.56, 29.41, 26.94, 22.62, 22.13. HRMS (ESI) m/z calculated for C₂₂H₁₉N₄O [M+H]⁺355.1553, found 355.1553.

### 3-methoxy-4-(8,9,10,11-tetrahydro-3H-naphtho[1,2-e]indazol-7-yl)benzonitrile

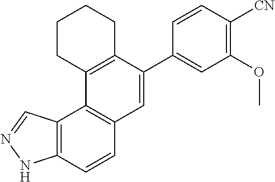 [SMILES: COc1cc(-c2cc3ccc4[nH]ncc4c3c3c2CCCC3)ccc1C#N]

Synthesized using Method A: Off white solid (0.719 g, 6.14 mmol, 33.1%). ¹H NMR (500 MHz, (CD₃)₂SO) δ 8.51 (s, 1H), 7.84 (q, J=8.9 Hz, 2H), 7.77 (d, J=_{7.8} Hz, 1H), 7.38 (s, 1H), 7.23 (d, J=7.9 Hz, 1H), 3.96 (s, 3H), 3.28 (t, J=6.7 Hz, 2H), 2.74 (t, J=6.3 Hz, 2H), 2.05-1.90 (m, 2H), 1.81-1.67 (m, 2H). ¹³C NMR (126 MHZ, (CD₃)₂SO) δ 161.15, 155.30, 148.08, 143.56, 142.68, 138.88, 136.39, 133.64, 129.57, 129.14, 122.31, 116.82, 116.44, 114.57, 113.51, 100.26, 56.99, 29.59, 28.50, 22.47, 22.41. HRMS (ESI) m/z calculated for C₂₂H₁₉N₄O [M+H]+355.1553, found 355.1552.

### 4-(8,9,10,11-tetrahydro-3H-pyrazolo[4,3-a]phenanthridin-7-yl)benzoic acid

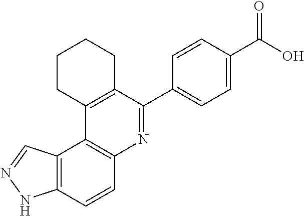 [SMILES: O=C(O)c1ccc(-c2nc3ccc4[nH]ncc4c3c3c2CCCC3)cc1]

Synthesized using Method B: Off white solid (0.812 g, 2 mmol 93.6%). ¹H NMR (500 MHz, (CD₃)₂SO) δ 8.75 (s, 1H), 8.26 (d, J=9.2 Hz, 1H), 8.19 (d, J=9.2 Hz, 1H), 8.15-8.11 (m, 2H), 7.85-7.80 (m, 2H), 3.46 (t, J=6.5 Hz, 2H), 2.76 (t, J=6.1 Hz, 2H), 2.03 (dd, J=6.0, 2.9 Hz, 2H), 1.83-1.76 (m, 2H). ¹³C NMR (126 MHZ, (CD₃)₂SO) δ 167.17, 151.31, 151.03, 140.41, 138.09, 137.05, 134.78, 132.69, 131.32, 130.42, 129.69, 123.45, 122.02, 120.11, 115.06, 30.67, 28.05, 21.85, 21.72. HRMS (ESI) m/z calculated for C₂₁H₁₈N₃O₂ [M+H]⁺344.1393, found 344.1392.

### 4-(9-methyl-3H-pyrazolo[4,3-f]quinolin-7-yl)benzoic acid

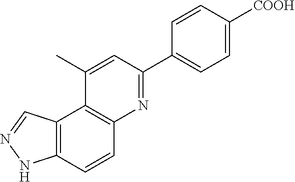 [SMILES: Cc1cc(-c2ccc(C(=O)O)cc2)nc2ccc3[nH]ncc3c12]

Synthesized using Method B: Off white solid (0.462 g, 3 mmol, 50.8%). ¹H NMR (500 MHz, (CD₃)₂SO) δ 8.64 (d, J=6.1 Hz, 1H), 8.33 (m, 2H), 8.22 (d, J=6.0 Hz, 1H), 8.15 (dd, J=9.2, 6.3 Hz, 1H), 8.09 (m, 3H), 3.01 (d, J=6.2 Hz, 3H). ¹³C NMR (126 MHZ, (CD₃)₂SO) δ 167.34, 151.05, 148.04, 143.57, 140.70, 139.95, 134.55, 132.31, 130.16, 128.18, 126.62, 122.86, 121.93, 118.55, 116.28, 23.05. HRMS (ESI) m/z calculated for C₁₈H₁₄N₃O₂ [M+H]⁺304.1081, found 304.1081.

### 4-(9-cyclopropyl-3H-pyrazolo[4,3-f]quinolin-7-yl)benzoic acid

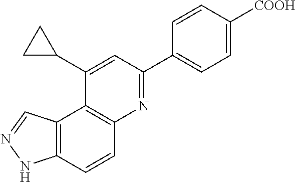 [SMILES: O=C(O)c1ccc(-c2cc(C3CC3)c3c(ccc4[nH]ncc43)n2)cc1]

Synthesized using Method B: Off white solid (0.464 g, 2.2 mmol, 64.1%). ¹H NMR (500 MHz, (CD₃)₂SO) δ 8.90 (s, 1H), 8.36 (d, J=8.2 Hz, 2H), 8.21 (d, J=9.2 Hz, 1H), 8.13-8.07 (m, 3H), 7.98 (s, 1H), 2.74 (d, J=5.4 Hz, 1H), 1.39 (dd, J=8.5, 2.4 Hz, 2H), 1.13 (dd, J=5.5, 2.2 Hz, 2H). ¹³C NMR (126 MHZ, (CD₃)₂SO) δ 167.35, 152.74, 151.36, 143.33, 140.68, 140.08, 135.01, 132.36, 130.10, 128.48, 126.47, 123.83, 118.75, 118.02, 115.98, 17.03, 8.58. HRMS (ESI) m/z calculated for C₂₀H₁₆N₃O₂ [M+H]⁺330.1237, found 330.1238.

### 4-(3,8,9,10,11,12-hexahydrocyclohepta[c]pyrazolo[4,3-f]quinolin-7-yl)benzoic acid

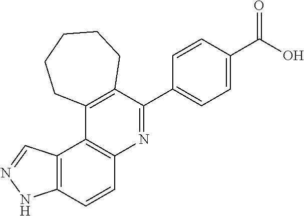 [SMILES: O=C(O)c1ccc(-c2nc3ccc4[nH]ncc4c3c3c2CCCCC3)cc1]

Synthesized using Method A: off white solid (0.522 g, 4 mmol, 36.5%). ¹H NMR (500 MHz, (CD₃)₂SO) δ 8.65 (s, 1H), 8.04 (d, J=7.8 Hz, 2H), 7.84 (d, J=9.2 Hz, 1H), 7.77 (d, J=9.1 Hz, 1H), 7.58 (d, J=7.8 Hz, 2H), 3.50 (d, J=7.2 Hz, 2H), 2.93 (d, J=8.4 Hz, 2H), 1.84 (d, J=9.5 Hz, 4H), 1.59 (d, J=7.8 Hz, 2H). ¹³C NMR (126 MHZ, (CD₃)₂SO) δ 167.57, 155.09, 148.75, 146.07, 144.73, 140.15, 135.20, 134.37, 134.16, 130.47, 129.81, 129.76, 129.46, 121.59, 116.02, 31.16, 31.10, 30.12, 27.28, 24.99. HRMS (ESI) m/z calculated for C₂₂H₂₀N₃O₂ [M+H]+358.1550, found 358.1550.

### 4-(3,8,10,11-tetrahydropyrano[3,4-c]pyrazolo[4,3-f]quinolin-7-yl)benzoic acid

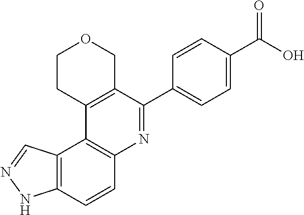 [SMILES: O=C(O)c1ccc(-c2nc3ccc4[nH]ncc4c3c3c2COCC3)cc1]

Synthesized using Method A: Off white solid (0.220 g, 0.76 mmol, 21.2%). ¹H NMR (500 MHz, (CD₃)₂SO) δ 8.68 (s, 1H), 8.13-7.99 (m, 4H), 7.76 (d, J=8.4 Hz, 2H), 4.77 (s, 2H), 4.17 (t, J=5.8 Hz, 2H), 3.49 (d, J=5.9 Hz, 2H). ¹³C NMR (126 MHZ, (CD₃)₂SO) δ 167.32, 150.67, 143.75, 141.15, 140.39, 140.21, 134.52, 132.11, 129.85, 129.79, 128.41, 125.87, 122.36, 118.50, 115.52, 66.34, 64.17, 29.11. HRMS (ESI) m/z calculated for C₂₀H₁₆N₃O₃ [M+H]⁺346.1186, found 346.1187.

### 4-(9,9-dioxido-3,8,10,11-tetrahydropyrazolo[4,3-f]thiopyrano[3,4-c]quinolin-7-yl)benzonitrile

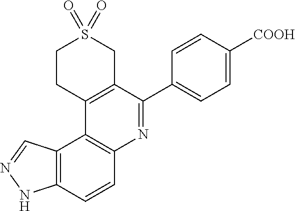 [SMILES: O=C(O)c1ccc(-c2nc3ccc4[nH]ncc4c3c3c2CS(=O)(=O)CC3)cc1]

Synthesized using Method A: Off white solid (1.68 g, 4.75 mmol, 89.7%). ¹H NMR (800 MHz, (CD₃)₂SO) δ 8.74 (s, 1H), 8.12 (d, J=8.1 Hz, 2H), 8.05 (d, J=9.0 Hz, 1H), 7.93 (d, J=9.0 Hz, 1H), 7.73 (d, J=8.1 Hz, 2H), 4.52 (s, 2H), 4.04 (d, J=6.7 Hz, 2H), 3.72 (d, J=6.5 Hz, 2H). ¹³C NMR (201 MHZ, (CD₃)₂SO) δ 167.37, 154.71, 143.89, 142.68, 140.28, 140.13, 135.10, 131.40, 129.94, 129.79, 128.36, 122.56, 121.45, 117.96, 115.45, 51.54, 45.94, 30.62. HRMS (ESI) m/z calculated for C₂₀H₁₆N₃O₄S [M+H]⁺394.0856, found 394.0856.

### 4-(3,8,9,10-tetrahydrocyclopenta[c]pyrazolo[4,3-f]quinolin-7-yl)benzoic acid

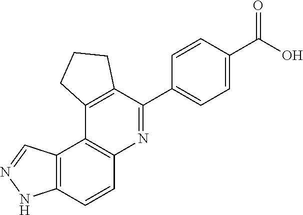 [SMILES: O=C(O)c1ccc(-c2nc3ccc4[nH]ncc4c3c3c2CCC3)cc1]

Synthesized using Method A: Off white solid (518 mg, 4 mmol, 39.3%). ¹H NMR (500 MHz, (CD₃)₂SO) δ 8.48 (s, 1H), 8.12-8.08 (m, 2H), 8.01-7.93 (m, 4H), 3.43 (t, J=7.6 Hz, 2H), 3.21 (t, J=7.5 Hz, 2H), 2.29-2.18 (m, 2H). ¹³C NMR (126 MHz, (CD₃)₂SO) δ 167.39, 153.03, 149.45, 142.46, 141.75, 139.61, 137.07, 133.84, 131.71, 129.75, 129.51, 126.17, 119.93, 117.67, 116.07, 34.02, 32.73, 24.73. HRMS (ESI) m/z calculated for C₂₀H₁₆N₃O₂ [M+H]⁺330.1237, found 330.1236.

### 4-(8,9-dihydro-3H-cyclobuta[c]pyrazolo[4,3-f]quinolin-7-yl)benzoic acid

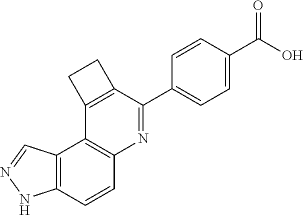 [SMILES: O=C(O)c1ccc(-c2nc3ccc4[nH]ncc4c3c3c2CC3)cc1]

Synthesized using Method A: Off white solid (0.030 g, 3 mmol, 3.2%). ¹H NMR (500 MHz, (CD₃)₂SO) δ 8.37 (d, J=0.9 Hz, 1H), 8.29-8.25 (m, 2H), 8.11-8.06 (m, 2H), 7.91-7.83 (m, 2H), 3.77 (dd, J=5.3, 2.6 Hz, 2H), 3.70-3.64 (m, 2H).¹³C NMR (126 MHZ, (CD₃)₂SO) & 167.53, 150.32, 146.59, 145.26, 141.88, 137.99, 131.57, 130.26, 130.16, 127.32, 117.98, 115.63, 115.59, 115.48, 115.01, 32.33, 30.41. HRMS (ESI) m/z calculated for C₁₉H₁₄N₃O₂ [M+H]⁺316.1080, found 316.1080.

### 4-(5-fluoro-8,9,10,11-tetrahydro-3H-pyrazolo[4,3-a]phenanthridin-7-yl)benzoic acid

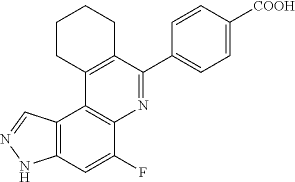 [SMILES: O=C(O)c1ccc(-c2nc3c(F)cc4[nH]ncc4c3c3c2CCCC3)cc1]

Synthesized using Method A: off white solid (0.145 g, 1.38 mmol, 29.1%). ¹H NMR (800 MHz, (CD₃)₂SO) δ 8.62 (s, 1H), 8.07 (d, J=7.7 Hz, 2H), 7.71 (dd, J=23.9, 8.8 Hz, 3H), 3.34-3.30 (m, 2H), 2.82-2.73 (m, 2H), 2.06-1.96 (m, 2H), 1.82-1.70 (m, 2H). ¹³C NMR (201 MHz, (CD₃)₂SO) δ 167.51, 157.66 (1J=250 Hz), 155.81, 144.92, 143.35, 134.62, 134.55, 134.49, 130.72, 130.68, 129.72, 129.43, 123.01, 112.66, 99.67 (2J=24 Hz), 29.63, 28.69, 22.27, 22.15. HRMS (ESI) m/z calculated for C₂₁H₁₇FN₃O₂ [M+H]⁺362.1299, found 362.1300.

### 3-fluoro-4-(8,9,10,11-tetrahydro-3H-pyrazolo[4,3-a]phenanthridin-7-yl)benzoic acid

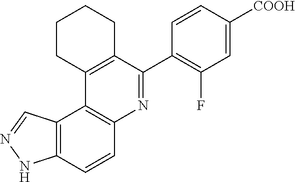 [SMILES: O=C(O)c1ccc(-c2nc3ccc4[nH]ncc4c3c3c2CCCC3)c(F)c1]

Synthesized using Method B: off white solid (1.58 g, 4.44 mmol, 98.5%). ¹H NMR (500 MHz, (CD₃)₂SO) δ 8.64 (s, 1H), 8.06 (d, J=22.2 Hz, 3H), 7.72 (d, J=11.2 Hz, 1H), 7.63 (d, J=8.1 Hz, 1H), 3.31 (t, J=6.6 Hz, 2H), 2.76 (t, J=6.2 Hz, 2H), 2.04-1.91 (m, 2H), 1.76 (q, J=6.0 Hz, 2H). ¹³C NMR (126 MHZ, (CD₃)₂SO) δ 167.55, 165.09, 162.00, 159.95, 157.23, 150.10, 132.37, 131.56, 130.83, 126.27, 123.27, 123.14, 121.70, 120.78, 120.70, 118.99 (2J=22 Hz), 115.01, 30.42, 28.01, 21.88, 21.75. HRMS (ESI) m/z calculated for C₂₁H₁₇FN₃O₂ [M+H]+362.1299, found 362.1300.

### 5-(8,9,10,11-tetrahydro-3H-pyrazolo[4,3-a]phenanthridin-7-yl)thiophene-2-carboxylic acid

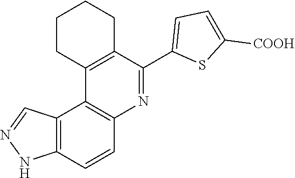 [SMILES: O=C(O)c1ccc(-c2nc3ccc4[nH]ncc4c3c3c2CCCC3)s1]

Synthesized using Method A: off white solid (0.545 g, 4 mmol, 39.0%). ¹H NMR (500 MHz, (CD₃)₂SO) δ 8.58 (s, 1H), 7.94 (dd, J=9.1, 0.9 Hz, 1H), 7.87 (d, J=9.2 Hz, 1H), 7.76 (d, J=3.8 Hz, 1H), 7.62 (d, J=4.0 Hz, 1H), 3.30 (t, J=6.5 Hz, 2H), 3.02 (t, J=6.2 Hz, 2H), 2.01-1.92 (m, 2H), 1.86-1.78 (m, 2H). ¹³C NMR (126 MHZ, (CD₃)₂SO) δ 163.22, 147.97, 146.86, 145.85, 141.63, 140.23, 136.54, 134.86, 133.59, 129.90, 129.82, 127.01, 122.73, 117.50, 115.69, 30.18, 28.68, 22.22, 22.03. HRMS (ESI) m/z calculated for C₁₉Hₐ₁₆N₃O₂S [M+H]⁺350.0958, found 350.0960.

### 3-(8,9,10,11-tetrahydro-3H-pyrazolo[4,3-a]phenanthridin-7-yl)benzoic acid

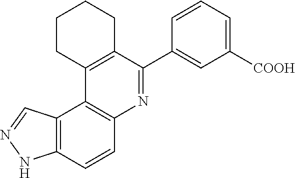 [SMILES: O=C(O)c1cccc(-c2nc3ccc4[nH]ncc4c3c3c2CCCC3)c1]

Synthesized using Method B: off white solid (0.453 g, 2 mmol, 65.9%). ¹H NMR (500 MHz, (CD₃)₂SO) δ 8.72 (s, 1H), 8.20 (t, J=1.8 Hz, 1H), 8.13-8.02 (m, 3H), 7.91 (m, 1H), 7.70 (t, J=7.7 Hz, 1H), 3.45 (t, J=6.5 Hz, 2H), 2.77 (t, J=6.2 Hz, 2H), 2.09-1.99 (m, 2H), 1.79 (m, 2H). ¹³C NMR (126 MHZ, (CD₃)₂SO) δ 167.28, 152.64, 140.35, 139.35, 136.86, 134.90, 134.08, 131.63, 130.72, 130.61, 130.47, 129.20, 128.96, 124.55, 123.02, 118.61, 115.51, 30.36, 28.37, 22.09, 22.01. HRMS (ESI) m/z calculated for C₂₁H₁₈N₃O₂ [M+H]⁺344.1393, found 344.1394.

### 3-methoxy-4-(8,9,10,11-tetrahydro-3H-naphtho[1,2-e]indazol-7-yl)benzoic acid

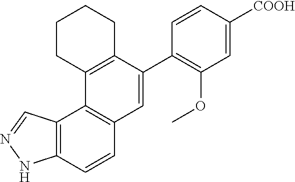 [SMILES: COc1cc(C(=O)O)ccc1-c1cc2ccc3[nH]ncc3c2c2c1CCCC2]

Synthesized using Method B: off white solid (0.193 g, 0.75 mmol, 68.2%). ¹H NMR (500 MHz, (CD₃)₂SO) δ 8.79 (s, 1H), 8.23 (d, J=9.2 Hz, 1H), 8.14 (d, J=9.2 Hz, 1H), 7.78-7.72 (m, 2H), 7.61 (d, J=7.7 Hz, 1H), 3.50 (d, J=6.8 Hz, 2H), 2.62 (t, J=6.3 Hz, 2H), 2.07-1.98 (m, 2H), 1.85-1.76 (m, 2H). ¹³C NMR (126 MHZ, (CD₃)₂SO) δ 167.10, 157.26, 151.83, 148.67, 140.49, 136.49, 134.97, 134.81, 132.47, 131.50, 126.57, 123.61, 122.16, 121.28, 120.55, 115.09, 112.62, 56.61, 30.58, 26.84, 21.83, 21.38. HRMS (ESI) m/z calculated for C₂₂H₂₁N₃O₃ [M+H]+374.1499, found 374.1497.

### 2-methoxy-4-(8,9,10,11-tetrahydro-3H-naphtho[1,2-e]indazol-7-yl)benzoic acid

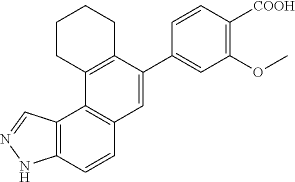 [SMILES: COc1cc(-c2cc3ccc4[nH]ncc4c3c3c2CCCC3)ccc1C(=O)O]

Synthesized using Method B: off white solid (814 mg, 2.31 mmol, 94.3%). ¹H NMR (800 MHz, (CD₃)₂SO) δ 8.52 (d, J=17.7 Hz, 1H), 7.86 (d, J=9.0 Hz, 1H), 7.84-7.80 (m, 1H), 7.78 (d, J=7.7 Hz, 1H), 7.39 (d, J=1.5 Hz, 1H), 7.23 (dd, J=7.7, 1.6 Hz, 1H), 3.96 (s, 3H), 3.23 (m, 2H), 2.72 (t, J=6.2 Hz, 2H), 1.95 (m, 2H), 1.77-1.68 (m, 2H). ¹³C NMR (201 MHz, (CD₃)₂SO) δ 161.02, 155.15, 147.91, 143.41, 142.64, 138.75, 136.30, 133.59, 129.43, 129.03, 122.32, 122.20, 116.82, 116.25, 114.53, 113.32, 100.02, 56.86, 29.47, 28.41, 22.32, 22.26. HRMS (ESI) m/z calculated for C₂₂H₂₁N₃O₃ [M+H]⁺374.1499, found 374.1497.

### tert-butyl 4-(4-(8,9,10,11-tetrahydro-3H-pyrazolo[4,3-a]phenanthridin-7-yl)benzoyl) piperazine-1-carboxylate

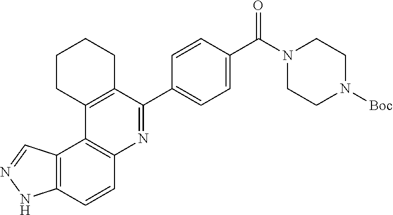 [SMILES: CC(C)(C)OC(=O)N1CCN(C(=O)c2ccc(-c3nc4ccc5[nH]ncc5c4c4c3CCCC4)cc2)CC1]

Synthesized using Method C: off white solid (100 mg, 0.5 mmol, 39.1%). ¹H NMR (800 MHz, (CD₃)₂SO) δ 8.59 (s, 1H), 7.93-7.78 (m, 2H), 7.64 (dd, J=8.7, 2.9 Hz, 2H), 7.52 (dd, J=8.1, 2.6 Hz, 2H), 3.70-3.54 (m, 2H), 3.54-3.38 (m, 5H), 2.80 (t, J=6.4 Hz, 2H), 2.01 (m, 2H), 1.76 (m, 2H), 1.42 (s, 9H). ¹³C NMR (201 MHZ, (CD₃)₂SO) δ 169.48, 161.41, 156.00, 154.23, 143.69, 142.45, 142.42, 138.47, 136.29, 135.38, 129.51, 129.19, 127.09, 121.98, 116.21, 114.53, 79.59, 47.31, 44.95, 43.73, 41.90, 29.58, 28.81, 28.43, 22.50, 22.41. HRMS (ESI) m/z calculated for C₃₀H₃₄N₅O₃ [M+H]⁺512.2656, found 512.2655.

### N-(2-(dimethylamino)ethyl)-4-(8,9,10,11-tetrahydro-3H-pyrazolo[4,3-a]phenanthridin-7-yl)benzamide (Compound HSH2177)

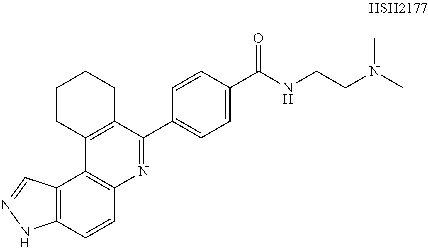 [SMILES: CN(C)CCNC(=O)c1ccc(-c2nc3ccc4[nH]ncc4c3c3c2CCCC3)cc1]

Synthesized using Method C: Off white solid (30 mg, 0.5 mmol, 14.65%) (¹H NMR (500 MHZ, (CD₃)₂SO) δ 8.58 (t, J=5.8 Hz, 2H), 7.96-7.90 (m, 2H), 7.88-7.79 (m, 2H), 7.67-7.62 (m, 2H), 3.40-3.27 (m, 7H), 2.77 (t, J=6.1 Hz, 2H), 2.39-2.25 (m, 7H), 2.13 (s, 3H), 2.00 (m, 4.4, 2.8 Hz, 2H), 1.74 (m, 2H), 1.68 (q, J=7.0 Hz, 2H). ¹³C NMR (126 MHZ, (CD₃)₂SO) δ 166.33, 156.14, 143.83, 142.54, 134.34, 129.64, 129.44, 129.27, 127.29, 122.07, 56.27, 55.23, 53.17, 46.20, 38.50, 29.67, 28.88, 26.69, 22.58, 22.49. HRMS (ESI) m/z calculated for C₂₅H₂₈N₅O [M+H]⁺414.2288, found 414.2285.

### N-(3-(4-methylpiperazin-1-yl) propyl)-4-(8,9,10,11-tetrahydro-3H-pyrazolo[4,3-a]phenanthridin-7-yl)benzamide (Compound HSH2195)

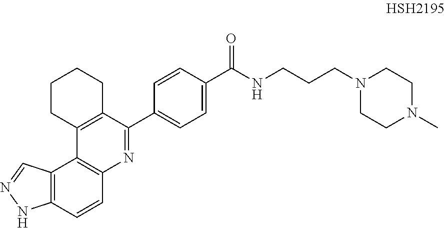 [SMILES: CN1CCN(CCCNC(=O)c2ccc(-c3nc4ccc5[nH]ncc5c4c4c3CCCC4)cc2)CC1]

Synthesized using Method C: Off white solid (10 mg, 0.5 mmol, 4.1%). ¹H NMR (500 MHz, (CD₃)₂SO) δ 8.58 (t, J=5.8 Hz, 2H), 7.96-7.90 (m, 2H), 7.88-7.79 (m, 2H), 7.67-7.62 (m, 2H), 3.31-3.28 (m, 3H), 2.77 (t, J=6.1 Hz, 2H), 2.42-2.23 (m, 8H), 2.13 (s, 3H), 2.00 (m, 2H), 1.74 (m, 2H), 1.68 (q, J=7.0 Hz, 2H). HRMS (ESI) m/z calculated for C₂₉H₃₅N₆O [M+H]⁺483.2867, found 483.2868.

### N-(cyclopropylmethyl)-4-(8,9,10,11-tetrahydro-3H-naphtho[1,2-e]indazol-7-yl)benzamide (Compound HSH3001)

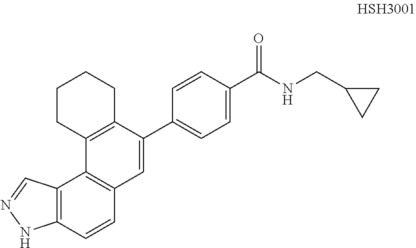 [SMILES: O=C(NCC1CC1)c1ccc(-c2cc3ccc4[nH]ncc4c3c3c2CCCC3)cc1]

Synthesized using Method C: Off white solid (20 mg, 0.5 mmol, 10.1%). ¹H NMR (500 MHz, (CD₃)₂SO) & 8.64 (t, J=5.7 Hz, 1H), 8.57 (s, 1H), 8.04-7.91 (m, 2H), 7.84 (q, J=12.4, 10.6 Hz, 2H), 7.69-7.57 (m, 2H), 3.18 (t, J=6.2 Hz, 2H), 2.76 (t, J=6.3 Hz, 2H), 2.03-1.95 (m, 2H), 1.73 (m, 2H), 1.06 (m, 1H), 0.51-0.36 (m, 2H), 0.31-0.17 (m, 2H). ¹³C NMR (126 MHz, (CD₃)₂SO) δ 166.33, 156.12, 143.73, 143.54, 142.68, 138.73, 136.42, 134.33, 129.53, 129.44, 129.34, 127.37, 122.03, 116.42, 114.57, 44.02, 29.68, 28.85, 22.55, 22.47, 11.53, 4.20, 3.80. HRMS (ESI) m/z calculated for C₂₅H₂₅N₄O [M+H]⁺397.2023, found 397.2021.

### N-(3-(piperidin-1-yl) propyl)-4-(8,9,10,11-tetrahydro-3H-pyrazolo[4,3-a]phenanthridin-7-yl)benzamide (Compound HSH3004)

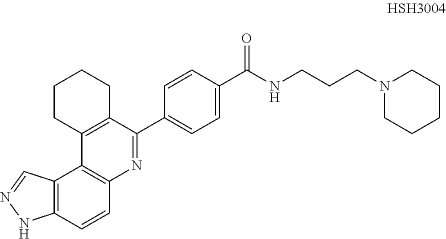 [SMILES: O=C(NCCCN1CCCCC1)c1ccc(-c2nc3ccc4[nH]ncc4c3c3c2CCCC3)cc1]

Synthesized using Method C: Off white solid (23 mg, 0.5 mmol, 9.8%). ¹H NMR (500 MHz, (CD₃)₂SO) δ 8.59 (s, 2H), 7.93 (s, 2H), 7.83 (d, J=19.7 Hz, 2H), 7.63 (s, 2H), 2.76 (s, 2H), 2.32 (s, 6H), 2.00 (s, 2H), 1.72 (d, J=19.4 Hz, 4H), 1.48 (s, 4H), 1.36 (s, 2H). HRMS (ESI) m/z calculated for C₂₉H₃₄N₅O [M+H]⁺468.2758, found 468.2759.

### N-(2-(dimethylamino)ethyl)-4-(8,9,10,11-tetrahydro-3H-8,11-methanopyrazolo[4,3-a]phenanthridin-7-yl)benzamide (Compound HSH3014)

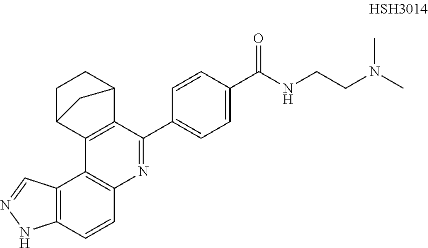 [SMILES: CN(C)CCNC(=O)c1ccc(-c2nc3ccc4[nH]ncc4c3c3c2C2CCC3C2)cc1]

Synthesized using Method F: Off white solid (10 mg, 0.5 mmol, 4.7%). ¹H NMR (500 MHz, (CD₃)₂SO) δ 8.69 (m, 1H), 8.47 (d, J=4.4 Hz, 1H), 8.28 (d, J=8.3 Hz, 1H), 8.02 (d, J=7.9 Hz, 2H), 7.91 (d, J=8.0 Hz, 2H), 7.84 (m, 1H), 7.29 (m, 1H), 4.36 (s, 1H), 3.75 (d, J=3.2 Hz, 1H), 2.87 (t, J=6.5 Hz, 2H), 2.53 (s, 6H), 2.21-2.14 (m, 2H), 1.85 (d, J=8.9 Hz, 1H), 1.71 (d, J=8.8 Hz, 1H), 1.36 (t, J=9.5 Hz, 1H), 1.23 (t, J=9.4 Hz, 1H). ¹³C NMR (126 MHZ, (CD₃)₂SO) & 166.68, 152.84, 148.81, 148.28, 142.68, 139.85, 139.78, 134.95, 134.15, 132.47, 129.57, 128.93, 128.20, 127.87, 119.88, 57.59, 49.98, 44.38, 43.32, 42.47, 36.55, 26.96, 25.42. HRMS (ESI) m/z calculated for C₂₆H₂₈N₅O [M+H]⁺426.2289, found 426.2287.

### N-(2-methoxyethyl)-4-(8,9,10,11-tetrahydro-3H-pyrazolo[4,3-a]phenanthridin-7-yl)benzamide (Compound HSH3016)

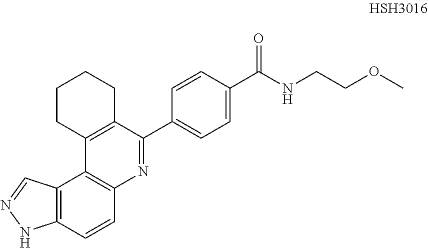 [SMILES: COCCNC(=O)c1ccc(-c2nc3ccc4[nH]ncc4c3c3c2CCCC3)cc1]

Synthesized using Method C: Off white solid (53 mg, 0.5 mmol, 26.25%). ¹H NMR (500 MHz, (CD₃)₂SO) δ 8.61 (t, J=5.1 Hz, 1H), 8.56 (s, 1H), 7.98-7.94 (m, 2H), 7.84 (d, J=4.9 Hz, 2H), 7.68-7.61 (m, 2H), 3.51-3.43 (m, 4H), 3.32 (d, J=4.0 Hz, 2H), 3.28 (s, 3H), 2.76 (t, J=6.1 Hz, 2H), 1.99 (m, 2H), 1.73 (m, 2H). ¹³C NMR (126 MHZ, (CD₃)₂SO) δ 166.50, 156.18, 143.93, 143.62, 142.54, 138.73, 136.42, 134.06, 129.65, 129.45, 129.33, 127.37, 121.99, 116.44, 114.52, 70.96, 58.42, 39.53, 29.68, 28.86, 22.57, 22.48. HRMS (ESI) m/z calculated for C₂₄H₂₅N₄O₂ [M+H]⁺401.1972, found 401.1970.

### 4-(8,9,10,11-tetrahydro-3H-pyrazolo[4,3-a]phenanthridin-7-yl)-N-(thiazol-2-yl)benzamide (Compound HSH3017)

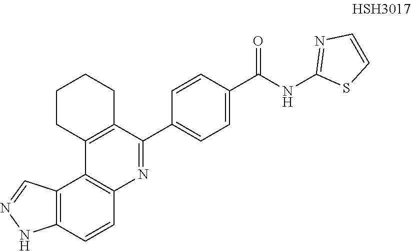 [SMILES: O=C(Nc1nccs1)c1ccc(-c2nc3ccc4[nH]ncc4c3c3c2CCCC3)cc1]

Synthesized using Method C: Off white (16 mg, 0.5 mmol, 7.5%). ¹H NMR (500 MHZ, (CD₃)₂SO) δ 8.59 (s, 1H), 8.20 (d, J=8.0 Hz, 2H), 7.92-7.83 (m, 2H), 7.73 (d, J=8.0 Hz, 2H), 7.57 (d, J=3.6 Hz, 1H), 7.29 (d, J=3.7 Hz, 1H), 2.80 (t, J=6.0 Hz, 2H), 2.07-1.94 (m, 2H), 1.85-1.65 (m, 2H).¹³C NMR (126 MHZ, (CD₃)₂SO) δ 165.35, 159.34, 155.86, 145.24, 143.70, 142.66, 138.72, 136.47, 136.42, 131.86, 129.77, 129.64, 129.34, 128.37, 122.12, 116.43, 114.65, 114.38, 29.69, 28.84, 22.57, 22.47. HRMS (ESI) m/z calculated for C₂₄H₂ON₅OS [M+H]⁺426.1383, found 426.1379.

### N-cyclopropyl-4-(8,9,10,11-tetrahydro-3H-pyrazolo[4,3-a]phenanthridin-7-yl)benzamide (Compound HSH3019)

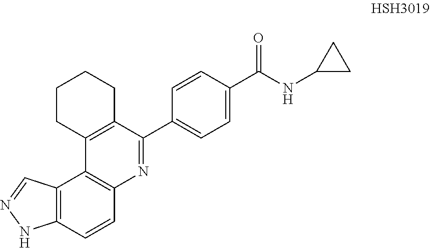 [SMILES: O=C(NC1CC1)c1ccc(-c2nc3ccc4[nH]ncc4c3c3c2CCCC3)cc1]

Synthesized using Method C: Off white solid (25.2 mg, 0.5 mmol, 13.2%). ¹H NMR (500 MHz, (CD₃)₂SO) δ 8.56 (s, 1H), 8.52 (d, J=4.3 Hz, 1H), 7.92 (d, J=7.9 Hz, 2H), 7.84 (t, J=9.7 Hz, 2H), 7.62 (d, J=7.9 Hz, 2H), 3.34 (s, 2H), 2.88 (m, 1H), 2.74 (t, J=6.1 Hz, 2H), 2.03-1.94 (m, 2H), 1.79-1.68 (m, 2H), 0.70 (m, 2H), 0.60 (m, 2H).¹³C NMR (126 MHZ, (CD₃)₂SO) δ 167.72, 156.13, 143.87, 143.63, 142.53, 138.74, 136.41, 134.09, 129.62, 129.39, 129.25, 127.33, 122.02, 116.41, 114.52, 29.65, 28.85, 23.59, 22.55, 22.47, 6.27. HRMS (ESI) m/z calculated for C₂₄H₂₃N₄O [M+H]⁺383.1866, found 383.1865.

### N-(3-methoxypropyl)-4-(8,9,10,11-tetrahydro-3H-pyrazolo[4,3-a]phenanthridin-7-yl)benzamide (Compound HSH3088)

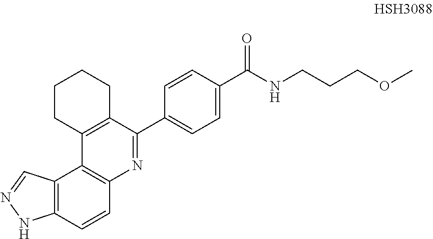 [SMILES: COCCCNC(=O)c1ccc(-c2nc3ccc4[nH]ncc4c3c3c2CCCC3)cc1]

Synthesized using Method F: Off white solid (13 mg, 0.5 mmol, 6.3%). ¹H NMR (500 MHz, (CD₃)₂SO) δ 8.55 (t, J=9.3 Hz, 1H), 7.95-7.91 (m, 2H), 7.88-7.82 (m, 2H), 7.65-7.60 (m, 2H), 7.52 (t, J=5.5 Hz, 1H), 3.24 (s, 3H), 3.21 (s, 2H), 3.15 (m, 2H), 2.76 (t, J=6.1 Hz, 2H), 2.00 (m, 2H), 1.76 (m, 6H).¹³C NMR (126 MHZ, (CD₃)₂SO) δ 166.43, 161.39, 156.19, 143.84, 143.64, 142.57, 138.73, 136.43, 134.28, 129.64, 129.44, 127.31, 122.01, 116.45, 114.53, 70.24, 69.75, 58.40, 42.45, 37.09, 29.69, 28.86, 22.56. HRMS (ESI) m/z calculated for C₂₅H₂₇N₄O₂ [M+H]⁺415.2129, found 415.2129.

### (4-methylpiperazin-1-yl) (4-(8,9,10,11-tetrahydro-3H-pyrazolo[4,3-a]phenanthridin-7-yl)phenyl) methanone (Compound HSH3107)

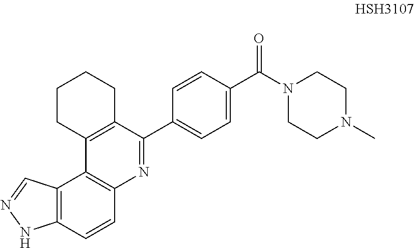 [SMILES: CN1CCN(C(=O)c2ccc(-c3nc4ccc5[nH]ncc5c4c4c3CCCC4)cc2)CC1]

Synthesized using Method D: Off white solid (510 mg, 4 mmol, 30%). ¹H NMR (500 MHZ, (CD₃)₂SO) δ 8.57 (s, 1H), 7.86 (d, J=9.0 Hz, 1H), 7.82 (s, 1H), 7.61 (d, J=7.9 Hz, 2H), 7.47 (d, J=8.0 Hz, 2H), 3.53 (s, 4H), 3.34 (t, J=6.8 Hz, 2H), 2.79 (t, J=6.3 Hz, 2H), 2.39-2.32 (m, 4H), 2.22 (s, 3H), 2.07-1.98 (m, 2H), 1.77 (m, 2H). ¹³C NMR (126 MHZ, (CD₃)₂SO) & 169.38, 156.15, 143.80, 142.53, 142.44, 135.81, 129.62, 129.53, 129.25, 128.15, 127.02, 122.09, 121.02, 116.34, 114.47, 55.02, 46.01, 29.66, 28.85, 22.61, 22.53. HRMS (ESI) m/z calculated for C₂₆H₂₈N₅O [M+H]⁺426.2289, found 426.2290.

### N-(2-(diethylamino)ethyl)-4-(8,9,10,11-tetrahydro-3H-pyrazolo[4,3-a]phenanthridin-7-yl)benzamide (Compound HSH3124)

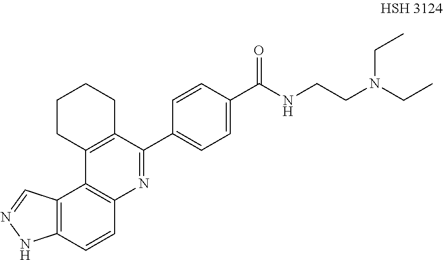 [SMILES: CCN(CC)CCNC(=O)c1ccc(-c2nc3ccc4[nH]ncc4c3c3c2CCCC3)cc1]

Synthesized using Method F: Off white solid (66 mg, 0.5 mmol, 30%). ¹H NMR (800 MHZ, (CD₃)₂SO) δ 8.59 (s, 1H), 8.39 (t, J=5.7 Hz, 1H), 7.99-7.91 (m, 2H), 7.87 (d, J=9.0 Hz, 1H), 7.83 (d, J=9.0 Hz, 1H), 7.68-7.63 (m, 2H), 3.40-3.32 (m, 4H), 2.78 (t, J=6.2 Hz, 2H), 2.60 (t, J=7.2 Hz, 2H), 2.53 (q, J=7.2 Hz, 4H), 2.04-2.00 (m, 2H), 1.78-1.73 (m, 2H), 1.01-0.97 (m, 6H). ¹³C NMR (201 MHZ, (CD₃)₂SO) δ 166.28, 156.08, 143.81, 142.44, 136.28, 134.30, 129.56, 129.36, 129.16, 127.16, 122.01, 116.24, 116.22, 116.18, 114.63, 51.99, 47.25, 38.12, 29.58, 28.76, 22.50, 22.42, 12.43. HRMS (ESI) m/z calculated for C₂₇H₃₂N₅O [M+H]⁺442.2601, found 442.2600.

### N-(2-(pyridin-2-yl)ethyl)-4-(8,9,10,11-tetrahydro-3H-pyrazolo[4,3-a]phenanthridin-7-yl)benzamide (Compound HSH3130)

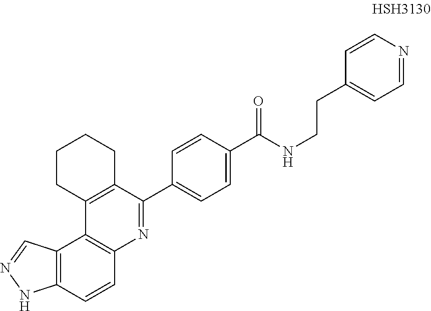 [SMILES: O=C(NCCc1ccncc1)c1ccc(-c2nc3ccc4[nH]ncc4c3c3c2CCCC3)cc1]

Synthesized using Method F: Off white solid (32 mg, 0.5 mmol, 14.3%). ¹H NMR (500 MHz, (CD₃)₂SO) δ 8.67 (m, 1H), 8.58 (s, 1H), 8.51-8.43 (m, 2H), 7.88 (m, 4H), 7.64 (d, J=8.2 Hz, 2H), 7.32-7.26 (m, 2H), 3.57 (q, J=6.7, 6.7, 6.6 Hz, 2H), 2.90 (t, J=7.1, 7.1 Hz, 2H), 2.76 (t, J=6.1, 6.1 Hz, 2H), 2.05-1.96 (m, 2H), 1.73 (m, 2H).¹³C NMR (126 MHZ, (CD₃)₂SO) δ 166.51, 156.13, 149.92, 148.98, 143.95, 143.68, 142.56, 134.11, 129.62, 129.48, 129.30, 129.11, 127.29, 124.76, 122.05, 120.96, 114.65, 114.47, 34.68, 29.67, 28.86, 22.57, 22.48. HRMS (ESI) m/z calculated for C₂₈H₂₆N₅O [M+H]⁺448.2132, found 448.2130.

### N-(3-morpholinopropyl)-4-(8,9,10,11-tetrahydro-3H-pyrazolo[4,3-a]phenanthridin-7-yl)benzamide (Compound HSH3146)

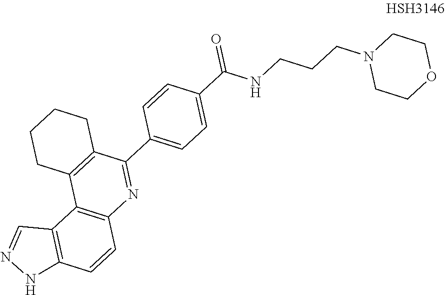 [SMILES: O=C(NCCCN1CCOCC1)c1ccc(-c2nc3ccc4[nH]ncc4c3c3c2CCCC3)cc1]

Synthesized using Method F: Off white solid (92.9 mg, 0.5 mmol, 39.5%). ¹H NMR (500 MHz, (CD₃)₂SO) δ 8.57 (t, J=5.4 Hz, 2H), 7.96-7.90 (m, 2H), 7.85 (q, J=9.0 Hz, 2H), 7.67-7.61 (m, 2H), 3.56 (t, J=4.6 Hz, 5H), 3.30 (d, J=6.5 Hz, 3H), 2.77 (t, J=6.1 Hz, 2H), 2.34 (d, J=6.7 Hz, 6H), 2.00 (m, 2H), 1.72 (m, 4H). ¹³C NMR (126 MHZ, (CD₃)₂SO) δ 166.37, 156.20, 143.83, 143.74, 143.59, 142.57, 136.42, 134.32, 129.64, 129.45, 129.34, 127.29, 122.00, 116.42, 114.52, 66.69, 56.59, 53.84, 38.29, 29.68, 28.86, 26.46, 22.57, 22.49. HRMS (ESI) m/z calculated for C₂₈H₃₂N₅O₂ [M+H]⁺470.2550, found 470.2548.

### N-(2-(piperidin-1-yl)ethyl)-4-(8,9,10,11-tetrahydro-3H-pyrazolo[4,3-a]phenanthridin-7-yl)benzamide (Compound HSH3117)

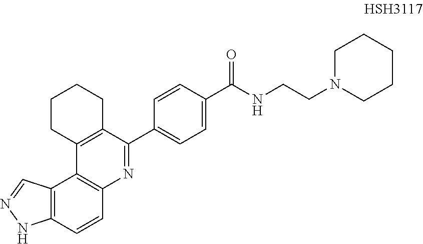 [SMILES: O=C(NCCN1CCCCC1)c1ccc(-c2nc3ccc4[nH]ncc4c3c3c2CCCC3)cc1]

Synthesized using Method F: Off white solid (10 mg, 0.5 mmol, 4.3%). ¹H NMR (800 MHz, DMSO-d₆) δ 8.59 (s, 1H), 8.40 (t, J=5.7 Hz, 1H), 7.95 (d, J=8.1 Hz, 2H), 7.87 (d, J=9.0 Hz, 1H), 7.82 (d, J=9.2 Hz, 1H), 7.65 (d, J=8.1 Hz, 2H), 3.42 (q, J=6.4 Hz, 2H), 3.33 (t, J=6.8 Hz, 2H), 2.78 (t, J=6.3 Hz, 2H), 2.47 (t, J=7.1 Hz, 2H), 2.44-2.34 (m, 4H), 2.01 (p, J=6.4 Hz, 2H), 1.75 (p, J=6.1 Hz, 2H), 1.50 (p, J=5.6 Hz, 4H), 1.38 (q, J=6.2 Hz, 2H). ¹³C NMR (201 MHz, DMSO-d₆) δ 166.29, 156.06, 143.89, 143.83, 143.74, 142.43, 136.09, 134.28, 129.55, 129.35, 129.15, 127.20, 122.00, 116.26, 114.66, 58.09, 54.49, 37.47, 29.58, 28.76, 26.03, 24.45, 22.50, 22.42. HRMS (ESI) m/z calculated for C₂₈H₃₂N₅O [M+H]⁺454.2601, found 454.2600.

### N-(2-(dimethylamino)ethyl)-4-(3,8,9,10-tetrahydrocyclopenta[c]pyrazolo[4,3-f]quinolin-7-yl)benzamide (Compound HSH3159)

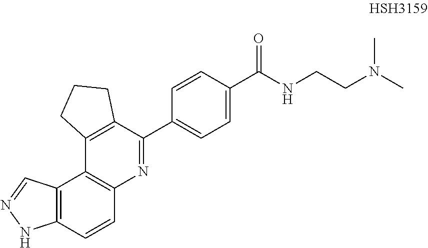 [SMILES: CN(C)CCNC(=O)c1ccc(-c2nc3ccc4[nH]ncc4c3c3c2CCC3)cc1]

Synthesized using Method F: Off white solid (10.8 mg, 0.25 mmol, 10.8%). ¹H NMR (500 MHz, (CD₃)₂SO) δ 8.95 (t, J=5.6 Hz, 1H), 8.51 (s, 1H), 8.07 (d, J=8.1 Hz, 2H), 7.99 (d, J=8.1 Hz, 2H), 7.90 (q, J=9.2 Hz, 2H), 3.67 (q, J=5.8 Hz, 2H), 3.50 (t, J=7.6 Hz, 2H), 2.83 (s, 6H), 2.28 (m, 2H). ¹³C NMR (126 MHZ, (CD₃)₂SO) δ 166.89, 151.26, 149.87, 143.36, 142.48, 141.78, 136.49, 136.34, 133.79, 133.45, 129.25, 128.86, 127.87, 119.54, 116.52, 56.49, 42.90, 35.12, 33.57, 33.10, 25.01. HRMS (ESI) m/z calculated for C₂₄H₂₆N₅O [M+H]⁺400.2132, found 400.2131.

### N-(cyanomethyl)-4-(3,8,9,10-tetrahydrocyclopenta[c]pyrazolo[4,3-f]quinolin-7-yl)benzamide (Compound HSH3160)

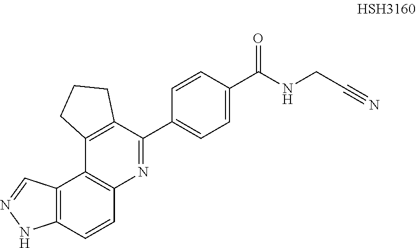 [SMILES: N#CCNC(=O)c1ccc(-c2nc3ccc4[nH]ncc4c3c3c2CCC3)cc1]

Synthesized using Method F: Off white solid (12.5 mg, 0.25 mmol, 13.6%). ¹H NMR (500 MHz, (CD₃)₂SO) δ 9.30 (t, J=5.5 Hz, 1H), 8.47 (s, 1H), 8.01 (d, J=1.2 Hz, 4H), 7.93 (d, J=10.0 Hz, 1H), 7.87 (d, J=9.0 Hz, 1H), 4.35 (d, J=5.3 Hz, 2H), 3.49 (t, J=7.6 Hz, 2H), 3.29 (t, J=7.5 Hz, 2H), 2.27 (m, 2H). ¹³C NMR (126 MHZ, (CD₃)₂SO) δ 166.88, 151.15, 149.88, 144.93, 143.81, 138.32, 136.42, 135.28, 132.76, 129.27, 129.09, 127.85, 119.50, 118.17, 116.69, 114.96, 33.56, 33.08, 28.27, 24.98. HRMS (ESI) m/z calculated for C₂₂H₁₈N₅O [M+H]⁺368.1506, found 368.1504.

### tert-butyl(2-(4-(8,9,10,11-tetrahydro-3H-pyrazolo[4,3-a]phenanthridin-7-yl)benzamido)ethyl) carbamate (Compound HSH3150)

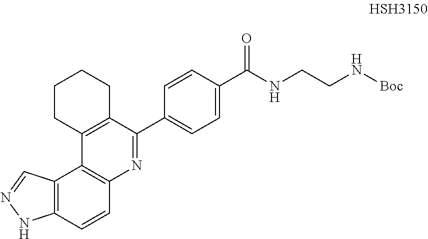 [SMILES: CC(C)(C)OC(=O)NCCNC(=O)c1ccc(-c2nc3ccc4[nH]ncc4c3c3c2CCCC3)cc1]

Synthesized using Method F: Off-white solid (170 mg, 0.5 mmol, 70%). ¹H NMR (500 MHz, (CD₃)₂SO) δ 8.56 (s, 1H), 8.41 (s, 1H), 7.95 (d, J=8.0 Hz, 2H), 7.84 (d, J=10.1 Hz, 2H), 7.63 (d, J=7.9 Hz, 2H), 6.78 (s, 1H), 3.34 (dt, J=13.1, 6.4 Hz, 4H), 3.15 (q, J=5.7 Hz, 2H), 2.76 (q, J=10.7, 8.4 Hz, 2H), 2.00 (h, J=9.1, 7.3 Hz, 2H), 1.75 (h, J=9.6, 7.7 Hz, 2H), 1.38 (s, 9H). ¹³C NMR (126 MHZ, (CD₃)₂SO) δ 166.74, 156.26, 156.20, 144.00, 143.78, 143.74, 142.53, 136.36, 136.31, 134.32, 129.66, 129.35, 129.24, 127.35, 122.08, 114.51, 78.22, 40.55, 29.65, 28.81, 28.80, 28.74, 22.58, 22.52. HRMS (ESI) m/z calculated for C₂₈H₃₂N₅O₃ [M+H]⁺486.2499, found 486.2494.

### N-(2-aminoethyl)-4-(8,9,10,11-tetrahydro-3H-pyrazolo[4,3-a]phenanthridin-7-yl)benzamide (Compound HSH3165)

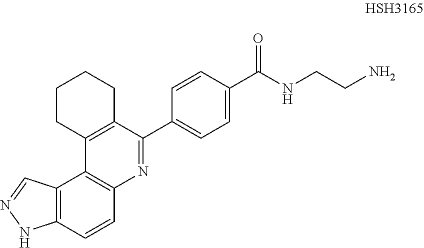 [SMILES: NCCNC(=O)c1ccc(-c2nc3ccc4[nH]ncc4c3c3c2CCCC3)cc1]

Synthesized using Method G: Off white solid (85 mg, 0.245 mmol, 89%). ¹H NMR (500 MHz, (CD₃)₂SO) δ 8.63 (s, 1H), 8.58 (s, 1H), 7.99 (d, J=8.1 Hz, 2H), 7.92 (s, 2H), 7.88-7.79 (m, 2H), 7.66 (d, J=8.1 Hz, 2H), 3.57 (q, J=6.0 Hz, 2H), 3.35 (d, J=6.5 Hz, 2H), 3.06 (t, J=6.2 Hz, 2H), 2.77 (t, J=6.2 Hz, 2H), 2.05-1.98 (m, 2H), 1.82-1.69 (m, 2H). HRMS (ESI) m/z calculated for C₂₃H₂₄N₅O [M+H]⁺386.1975, found 386.1973.

### N-(2-(methylsulfonyl)ethyl)-4-(8,9,10,11-tetrahydro-3H-pyrazolo[4,3-a]phenanthridin-7-yl)benzamide (Compound HSH3175)

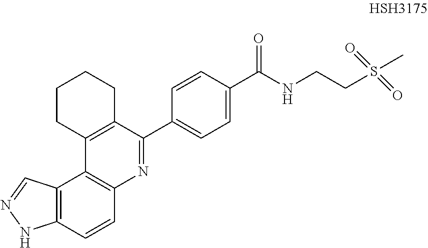 [SMILES: CS(=O)(=O)CCNC(=O)c1ccc(-c2nc3ccc4[nH]ncc4c3c3c2CCCC3)cc1]

Synthesized using Method E: Off white solid (6.7 mg, 0.5 mmol, 3%). ¹H NMR (500 MHZ, (CD₃)₂SO) δ 8.81 (t, J=5.6 Hz, 1H), 8.57 (s, 1H), 7.99-7.88 (m, 2H), 7.85 (q, J=9.2 Hz, 2H), 7.70-7.60 (m, 2H), 3.76-3.63 (m, 2H), 3.41 (t, J=6.9 Hz, 2H), 3.05 (s, 3H), 2.76 (t, J=6.1 Hz, 2H), 2.00 (m, 2H), 1.74 (m, 2H). ¹³C NMR (126 MHZ, (CD₃)₂SO) δ 166.71, 156.13, 144.19, 143.62, 142.60, 138.74, 136.43, 133.68, 129.64, 129.56, 129.34, 127.37, 122.02, 116.44, 114.56, 53.38, 41.26, 33.90, 29.68, 28.85, 22.56, 22.47. HRMS (ESI) m/z calculated for C₂₄H₂₅N₄O₃S [M+H]⁺449.1642, found 449.1642.

### N-(2-sulfamoylethyl)-4-(8,9,10,11-tetrahydro-3H-pyrazolo[4,3-a]phenanthridin-7-yl)benzamide (Compound HSH3184)

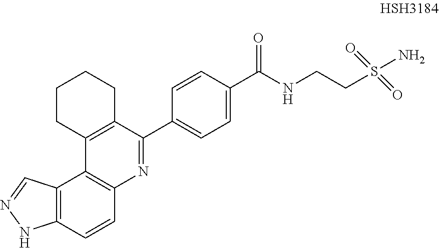 [SMILES: NS(=O)(=O)CCNC(=O)c1ccc(-c2nc3ccc4[nH]ncc4c3c3c2CCCC3)cc1]

Synthesized using Method F: Off white solid (10 mg, 0.5 mmol, 2.23%). ¹H NMR (500 MHz, (CD₃)₂SO) δ 8.58 (s, 2H), 7.97-7.93 (m, 2H), 7.89-7.79 (m, 2H), 7.67-7.62 (m, 2H), 3.77-3.67 (m, 2H), 3.34 (t, J=6.5 Hz, 2H), 3.29 (dd, J=8.1, 6.2 Hz, 2H), 2.77 (t, J=6.2 Hz, 2H), 2.07-1.98 (m, 2H), 1.76 (m, 2H). ¹³C NMR (126 MHz, (CD₃)₂SO) δ 166.75, 156.12, 144.22, 143.88, 142.57, 133.96, 129.77, 129.64, 129.48, 129.23, 128.11, 127.34, 127.11, 122.14, 116.28, 54.24, 35.33, 29.66, 28.80, 22.58, 22.52. HRMS (ESI) m/z calculated for C₂₃H₂₄N₅O₃S [M+H]⁺450.1594, found 450.1591.

### (4-isopropylpiperazin-1-yl) (4-(8,9,10,11-tetrahydro-3H-pyrazolo[4,3-a]phenanthridin-7-yl)phenyl) methanone (Compound HSH3182)

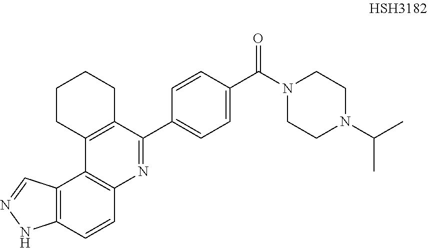 [SMILES: CC(C)N1CCN(C(=O)c2ccc(-c3nc4ccc5[nH]ncc5c4c4c3CCCC4)cc2)CC1]

Synthesized using Method F: Off white solid (56 mg, 0.5 mmol, 24.6%). ¹H NMR (500 MHz, (CD₃)₂SO) δ 8.56 (s, 1H), 7.91-7.77 (m, 2H), 7.68-7.57 (m, 2H), 7.47 (d, J=8.0 Hz, 2H), 3.51 (s, 4H), 2.78 (t, J=6.1 Hz, 2H), 2.67 (m, 1H), 2.46 (d, J=5.3 Hz, 3H), 2.01 (m, 2H), 1.81-1.71 (m, 2H), 0.97 (d, J=6.5 Hz, 6H). ¹³C NMR (126 MHZ, (CD₃)₂SO) δ 169.32, 156.19, 143.83, 143.77, 142.59, 142.37, 136.23, 135.81, 129.63, 129.50, 129.29, 127.06, 122.09, 116.35, 114.55, 54.23, 48.71, 29.66, 28.84, 22.58, 22.51, 18.56. HRMS (ESI) m/z calculated for C₂₈H₃₂N₅O [M+H]⁺454.2601, found 454.2598.

### N-(prop-2-yn-1-yl)-4-(8,9,10,11-tetrahydro-3H-pyrazolo[4,3-a]phenanthridin-7-yl)benzamide (Compound HSH3199)

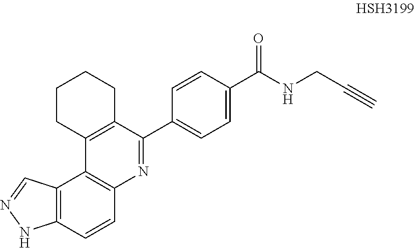 [SMILES: C#CCNC(=O)c1ccc(-c2nc3ccc4[nH]ncc4c3c3c2CCCC3)cc1]

Synthesized using Method F: Off white solid (14 mg, 0.2 mmol, 18.4%). (¹H NMR (500 MHz, (CD₃)₂SO) δ 9.02 (t, J=5.7 Hz, 1H), 8.59 (s, 1H), 7.96 (d, J=8.0 Hz, 2H), 7.85 (t, J=11.3 Hz, 2H), 7.65 (d, J=8.0 Hz, 2H), 4.09 (d, J=6.5 Hz, 2H), 3.23 (s, 1H), 3.13 (s, 1H), 2.76 (t, J=6.3 Hz, 2H), 2.06-1.94 (m, 2H), 1.74 (d, J=5.7 Hz, 2H). HRMS (ESI) m/z calculated for C₂₄H₂₁N₄O [M+H]⁺381.1710, found 381.1709.

### N-(1-((dimethylamino)methyl)cyclopropyl)-4-(8,9,10,11-tetrahydro-3H-pyrazolo[4,3-a]phenanthridin-7-yl)benzamide (Compound HSH3192)

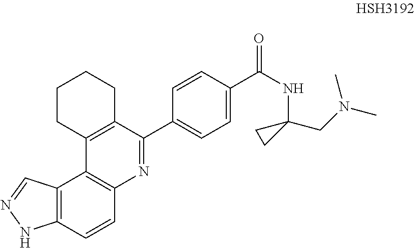 [SMILES: CN(C)CC1(NC(=O)c2ccc(-c3nc4ccc5[nH]ncc5c4c4c3CCCC4)cc2)CC1]

Synthesized using Method F: Off white solid (18.2 mg, 0.5 mmol, 8.3%). ¹H NMR (500 MHz, (CD₃)₂SO) δ 8.59 (d, J=9.7 Hz, 2H), 7.98-7.90 (m, 2H), 7.84 (d, J=11.5 Hz, 2H), 7.65-7.55 (m, 2H), 2.75 (t, J=6.1 Hz, 2H), 2.53 (s, 2H), 2.26 (s, 6H), 2.00 (m, 2H), 1.73 (m, 2H), 0.85-0.76 (m, 2H), 0.68 (q, J=4.8 Hz, 2H). ¹³C NMR (126 MHZ, (CD₃)₂SO) δ 166.90, 156.21, 143.84, 143.68, 142.57, 138.78, 138.70, 136.40, 134.40, 129.64, 129.31, 127.48, 122.05, 116.40, 114.54, 63.91, 49.07, 45.90, 31.57, 29.67, 28.88, 22.57, 22.49, 12.82. HRMS (ESI) m/z calculated for C₂₇H₃₀NO [M+H]⁺440.2444, found 440.2443.

### N-(2-(dimethylamino)ethyl)-4-(3,8,9,10,11,12-hexahydrocyclohepta[c]pyrazolo[4,3-f]quinolin-7-yl)benzamide (Compound HSH3194)

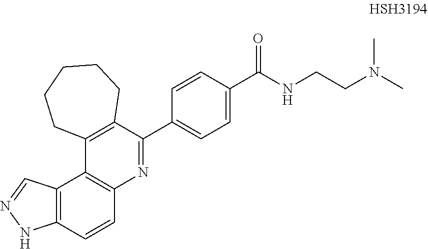 [SMILES: CN(C)CCNC(=O)c1ccc(-c2nc3ccc4[nH]ncc4c3c3c2CCCCC3)cc1]

Synthesized using Method F: off white solid (13.5 mg, 0.5 mmol, 6.3%). ¹H NMR (500 MHz, (CD₃)₂SO) δ 8.62 (s, 1H), 8.32 (q, J=5.7, 4.9 Hz, 1H), 7.96-7.90 (m, 2H), 7.84 (dd, J=9.1, 0.9 Hz, 1H), 7.78 (d, J=9.1 Hz, 1H), 7.60-7.51 (m, 2H), 3.54 (t, J=4.9 Hz, 2H), 3.41 (td, J=6.7, 5.4 Hz, 2H), 3.00-2.96 (m, 2H), 2.22 (s, 6H), 1.93-1.82 (m, 4H), 1.63 (m, 2H). 13C NMR (126 MHZ, (CD₃)₂SO) δ 166.51, 155.33, 148.69, 146.38, 144.72, 144.41, 139.87, 135.25, 134.27, 129.78, 129.49, 127.32, 121.51, 118.84, 116.05, 58.62, 45.59, 37.93, 31.20, 31.10, 30.14, 27.30, 25.03. HRMS (ESI) m/z calculated for C₂₆H₃₀N₅O [M+H]⁺428.2444, found 428.2442.

### N-(2-(dimethylamino)ethyl)-4-(3,8,10,11-tetrahydropyrano[3,4-c]pyrazolo[4,3-f]quinolin-7-yl)benzamide (Compound HSH3195)

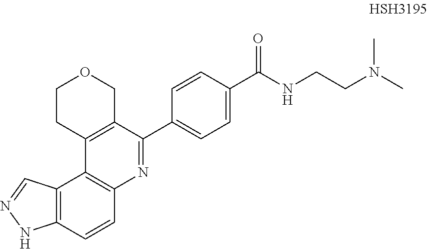 [SMILES: CN(C)CCNC(=O)c1ccc(-c2nc3ccc4[nH]ncc4c3c3c2COCC3)cc1]

Synthesized using Method F: Off-white solid (57 mg, 0.5 mmol, 27.4%). ¹H NMR (500 MHz, (CD₃)₂SO) δ 8.59 (s, 1H), 8.34 (t, J=5.6 Hz, 1H), 7.98-7.83 (m, 4H), 7.67-7.64 (m, 2H), 4.78 (d, J=1.5 Hz, 2H), 4.16 (t, J=5.9 Hz, 2H), 3.43-3.37 (m, 4H), 2.46 (d, J=6.8 Hz, 2H), 2.21 (s, 6H). ¹³C NMR (126 MHZ, (CD₃)₂SO) δ 166.36, 161.45, 153.03, 144.49, 142.38, 139.50, 134.89, 129.53, 129.16, 127.53, 127.50, 127.05, 121.54, 118.63, 116.09, 66.84, 64.42, 58.66, 45.65, 37.99, 28.74. HRMS (ESI) m/z calculated for C₂₄H₂₆N₅O₂ [M+H]⁺416.2081, found 416.2080.

### 4-(8,9-dihydro-3H-cyclobuta[c]pyrazolo[4,3-f]quinolin-7-yl)-N-(2-(dimethylamino)ethyl)benzamide (Compound HSH3197)

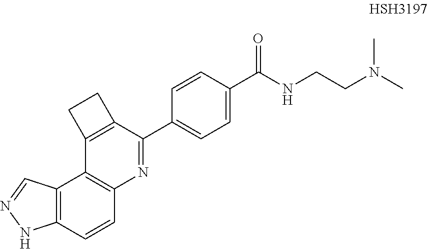 [SMILES: CN(C)CCNC(=O)c1ccc(-c2nc3ccc4[nH]ncc4c3c3c2CC3)cc1]

Synthesized using Method F: Off white solid (9 mg, 0.095 mmol, 24.5%). ¹H NMR (500 MHz, (CD₃)₂SO) δ 8.39-8.32 (m, 2H), 8.26-8.20 (m, 2H), 8.02-7.96 (m, 2H), 7.93-7.81 (m, 2H), 3.79-3.74 (m, 2H), 3.70-3.64 (m, 2H), 3.42 (td, J=6.7, 5.5 Hz, 2H), 2.52 (t, J=6.8 Hz, 2H), 2.26 (s, 6H). ¹³C NMR (126 MHZ, (CD₃)₂SO) δ 166.42, 161.50, 150.26, 146.88, 145.25, 140.35, 137.83, 135.37, 130.23, 130.15, 128.15, 127.23, 127.10, 117.87, 115.04, 58.54, 45.48, 37.81, 32.32, 30.39. HRMS (ESI) m/z calculated for C₂₃H₂₄N₅O [M+H]⁺386.1975, found 386.1974.

### N-(2-(pyrrolidin-1-yl)ethyl)-4-(8,9,10,11-tetrahydro-3H-pyrazolo[4,3-a]phenanthridin-7-yl)benzamide (Compound HSH3180)

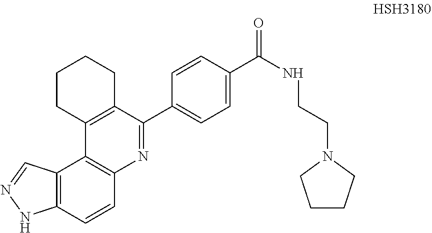 [SMILES: O=C(NCCN1CCCC1)c1ccc(-c2nc3ccc4[nH]ncc4c3c3c2CCCC3)cc1]

Synthesized using Method F: Off white solid (9 mg, 0.5 mmol, 4.1%). ¹H NMR (500 MHz, (CD₃)₂SO) δ 8.56 (s, 1H), 8.34 (t, J=5.7 Hz, 1H), 7.98-7.93 (m, 2H), 7.87-7.78 (m, 2H), 7.66-7.61 (m, 2H), 3.43 (d, J=5.8 Hz, 2H), 3.32 (t, J=6.6 Hz, 2H), 2.76 (t, J=6.1 Hz, 2H), 2.62 (t, J=6.9 Hz, 2H), 2.53-2.47 (m, 4H), 2.04-1.95 (m, 2H), 1.79-1.72 (m, 2H), 1.72-1.65 (m, 4H). ¹³C NMR (126 MHZ, (CD₃)₂SO) δ 166.44, 161.41, 156.16, 143.96, 143.90, 142.49, 140.03, 134.43, 129.65, 129.39, 129.19, 127.30, 122.11, 116.29, 115.28, 55.38, 54.10, 39.25, 29.64, 28.82, 23.75, 22.59, 22.52. HRMS (ESI) m/z calculated for C₂₇H₃₀N₅O [M+H]⁺440.2444, found 440.2443.

### (4-(3,8,9,10,11,12-hexahydrocyclohepta[c]pyrazolo[4,3-f]quinolin-7-yl)phenyl) (4-methylpiperazin-1-yl) methanone (Compound HSH3181)

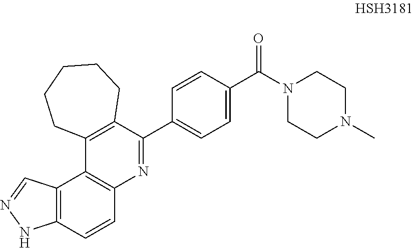 [SMILES: CN1CCN(C(=O)c2ccc(-c3nc4ccc5[nH]ncc5c4c4c3CCCCC4)cc2)CC1]

Synthesized using Method F: Off white solid (74 mg, 0.4 mmol, 42.1%). ¹H NMR (800 MHz, (CD₃)₂SO) δ 8.63 (s, 1H), 7.85 (d, J=9.0 Hz, 1H), 7.79 (s, 1H), 7.54 (d, J=8.0 Hz, 2H), 7.48 (d, J=8.1 Hz, 2H), 3.61 (s, 2H), 3.57-3.50 (m, 3H), 3.02-2.96 (m, 2H), 2.40-2.26 (m, 4H), 2.20 (s, 3H), 1.87 (m, 4H), 1.63 (m, 2H). ¹³C NMR (201 MHZ, (CD₃)₂SO) δ 169.22, 161.12, 155.16, 148.67, 144.49, 142.75, 138.93, 135.49, 135.25, 129.65, 129.57, 127.02, 121.36, 116.07, 114.83, 55.12, 54.94, 54.28, 45.96, 45.05, 31.26, 31.00, 30.06, 27.28, 24.89. HRMS (ESI) m/z calculated for C₂₇H₃₀N₅O [M+H]⁺440.2445, found 440.2444.

### (4-(5-fluoro-8,9,10,11-tetrahydro-3H-pyrazolo[4,3-a]phenanthridin-7-yl)phenyl) (4-methylpiperazin-1-yl) methanone (Compound HSH3183)

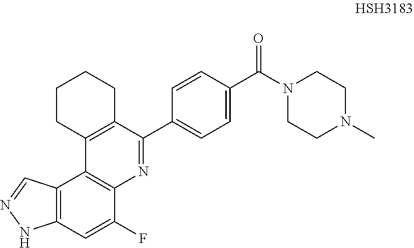 [SMILES: CN1CCN(C(=O)c2ccc(-c3nc4c(F)cc5[nH]ncc5c4c4c3CCCC4)cc2)CC1]

Synthesized using Method F: Off white solid (37 mg, 0.38 mmol, 22%). ¹H NMR (800 MHz, (CD₃)₂SO) δ 8.55 (s, 1H), 7.71 (d, J=10.0 Hz, 1H), 7.64 (d, J=7.6 Hz, 2H), 7.50 (d, J=7.0 Hz, 2H), 3.65 (s, 2H), 3.30 (s, 2H), 2.79 (d, J=6.7 Hz, 2H), 2.34 (d, J=68.6 Hz, 4H), 2.20 (d, J=6.3 Hz, 3H), 1.99 (q, J=6.6, 6.1 Hz, 2H), 1.74 (q, J=6.2 Hz, 2H). ¹³C NMR (201 MHZ, (CD₃)₂SO) δ 169.18, 157.78 (1J=250 Hz), 156.11, 142.96, 141.87, 136.43, 135.83, 134.28, 130.75, 130.71, 129.53, 127.01, 122.72, 112.86, 98.40, 55.14, 54.64, 47.52, 45.98, 41.85, 29.59, 28.83, 22.31, 22.19. HRMS (ESI) m/z calculated for C₂₆H₂₇FN₅O [M+H]⁺444.2194, found 444.2196.

### piperidin-1-yl(4-(8,9,10,11-tetrahydro-3H-pyrazolo[4,3-a]phenanthridin-7-yl)phenyl) methanone (Compound HSH3185)

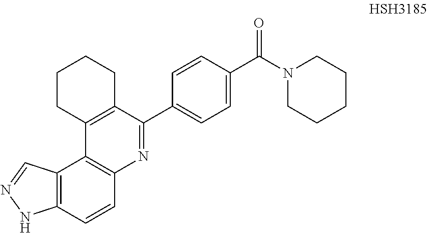 [SMILES: O=C(c1ccc(-c2nc3ccc4[nH]ncc4c3c3c2CCCC3)cc1)N1CCCCC1]

Synthesized using Method F: Off white solid (14 mg, 0.5 mmol, 6.8%). ¹H NMR (800 MHz, (CD₃)₂SO) δ 8.57 (s, 1H), 7.91-7.83 (m, 2H), 7.63 (d, J=8.1 Hz, 2H), 7.47 (d, J=8.0 Hz, 2H), 3.61 (s, 2H), 3.37 (t, J=6.7 Hz, 4H), 2.81 (t, J=6.2 Hz, 2H), 2.02 (td, J=6.1, 3.3 Hz, 2H), 1.81-1.72 (m, 2H), 1.67-1.62 (m, 2H), 1.55 (m, 4H). ¹³C NMR (201 MHz, (CD₃)₂SO) δ 169.15, 156.17, 143.61, 142.44, 142.08, 138.69, 136.30, 136.27, 129.55, 129.44, 129.26, 126.68, 121.90, 116.40, 114.40, 40.50, 29.62, 28.82, 25.73, 24.47, 22.52, 22.44. HRMS (ESI) m/z calculated for C₂₆H₂₇N₄O [M+H]⁺411.2179, found 411.2178.

### N-cyclohexyl-4-(8,9,10,11-tetrahydro-3H-pyrazolo[4,3-a]phenanthridin-7-yl)benzamide (Compound HSH3186)

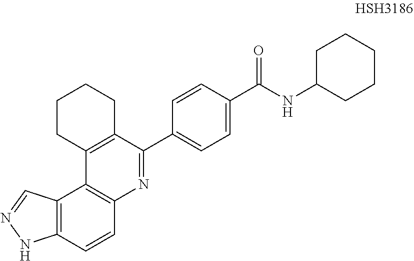 [SMILES: O=C(NC1CCCCC1)c1ccc(-c2nc3ccc4[nH]ncc4c3c3c2CCCC3)cc1]

Synthesized using Method F: Off white solid (13 mg, 0.5 mmol, 6.5%). ¹H NMR (800 MHz, (CD₃)₂SO) δ 8.59 (s, 1H), 8.25-8.20 (m, 1H), 8.00-7.94 (m, 2H), 7.90-7.82 (m, 2H), 7.64 (td, J=4.7, 2.4 Hz, 2H), 3.82 (tt, J=7.5, 4.0 Hz, 1H), 3.27 (d, J=5.7 Hz, 2H), 2.85-2.73 (m, 2H), 2.03 (q, J=6.2 Hz, 2H), 1.87 (d, J=11.4 Hz, 2H), 1.80-1.73 (m, 4H), 1.66-1.60 (m, 1H), 1.42-1.29 (m, 4H), 1.20-1.13 (m, 1H). ¹³C NMR (126 MHZ, (CD₃)₂SO) δ 165.56, 156.21, 143.73, 143.64, 142.54, 138.71, 136.39, 134.62, 134.55, 129.65, 129.32, 127.45, 122.03, 116.42, 114.54, 48.86, 32.92, 29.67, 28.87, 25.77, 25.45, 22.58, 22.50. HRMS (ESI) m/z calculated for C₂₇H₂₉N₄O [M+H]⁺425.2336, found 425.2336.

### 1-(4-(4-(8,9,10,11-tetrahydro-3H-pyrazolo[4,3-a]phenanthridin-7-yl)benzoyl) piperazin-1-yl) ethan-1-one (Compound HSH3187)

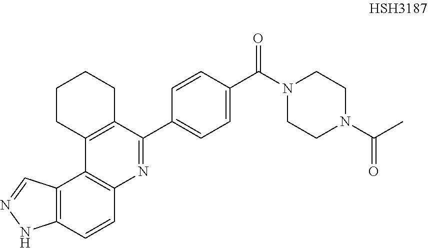 [SMILES: CC(=O)N1CCN(C(=O)c2ccc(-c3nc4ccc5[nH]ncc5c4c4c3CCCC4)cc2)CC1]

Synthesized using Method F: Off white solid (13.8 mg, 0.5 mmol, 5.7%). ¹H NMR (500 MHz, (CD₃)₂SO) δ 8.56 (s, 1H), 7.86 (d, J=9.1 Hz, 1H), 7.81 (d, J=9.8 Hz, 1H), 7.65-7.61 (m, 2H), 7.54-7.49 (m, 2H), 3.57-3.50 (m, 6H), 3.34 (q, J=9.8, 7.4 Hz, 4H), 2.79 (t, J=6.1 Hz, 2H), 2.05-1.97 (m, 5H), 1.79-1.73 (m, 2H). ¹³C NMR (126 MHZ, (CD₃)₂SO) δ 169.72, 169.08, 156.13, 143.82, 142.60, 142.57, 135.50, 129.62, 129.57, 129.27, 127.15, 122.10, 118.03, 116.51, 116.36, 114.50, 46.07, 41.38, 29.65, 28.84, 22.58, 22.52, 21.57. HRMS (ESI) m/z calculated for C₂₇H₂₈N₅O₂ [M+H]⁺454.2237, found 454.2241.

### (4-methylpiperazin-1-yl) (5-(8,9,10,11-tetrahydro-3H-pyrazolo[4,3-a]phenanthridin-7-yl)thiophen-2-yl) methanone (Compound HSH3200)

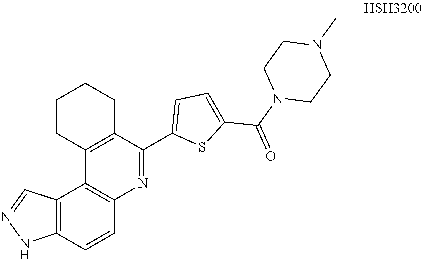 [SMILES: CN1CCN(C(=O)c2ccc(-c3nc4ccc5[nH]ncc5c4c4c3CCCC4)s2)CC1]

Synthesized using Method F: Off white solid (17 mg, 0.5 mmol, 7.9%). ¹H NMR (800 MHz, (CD₃)₂SO) δ 8.56 (s, 1H), 7.88 (d, J=9.0 Hz, 1H), 7.83-7.76 (m, 1H), 7.57 (d, J=3.9 Hz, 1H), 7.40 (d, J=3.8 Hz, 1H), 3.68 (t, J=5.2 Hz, 4H), 3.34 (s, 2H), 3.11 (t, J=6.2 Hz, 2H), 2.39 (t, J=5.2 Hz, 4H), 2.23 (s, 3H), 2.05-2.00 (m, 2H), 1.89-1.85 (m, 2H). ¹³C NMR (201 MHz, (CD₃)₂SO) δ 162.76, 148.32, 147.82, 143.33, 143.23, 138.76, 138.42, 136.38, 129.71, 129.21, 128.95, 127.50, 121.98, 116.25, 114.98, 54.98, 45.93, 29.89, 28.86, 22.43, 22.19. HRMS (ESI) m/z calculated for C₂₄H₂₆N₅OS [M+H]⁺432.1852, found 432.1853.

### (4-methylpiperazin-1-yl) (3-(8,9,10,11-tetrahydro-3H-pyrazolo[4,3-a]phenanthridin-7-yl)phenyl) methanone (Compound HSH3201)

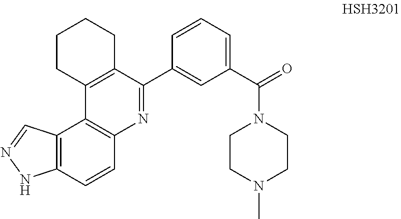 [SMILES: CN1CCN(C(=O)c2cccc(-c3nc4ccc5[nH]ncc5c4c4c3CCCC4)c2)CC1]

Synthesized using Method F: Off white solid (20 mg, 0.5 mmol, 9.4%). ¹H NMR (800 MHz, (CD₃)₂SO) δ 8.57 (s, 1H), 7.91-7.80 (m, 2H), 7.65 (d, J=7.8 Hz, 1H), 7.55 (d, J=7.2 Hz, 2H), 7.44 (d, J=7.8 Hz, 1H), 3.78-3.55 (m, 4H), 2.79 (t, J=6.3 Hz, 2H), 2.43-2.30 (m, 5H), 2.19 (s, 3H), 2.04-1.96 (m, 2H), 1.81-1.71 (m, 2H).¹³C NMR (201 MHZ, (CD₃)₂SO) δ 169.21, 155.93, 143.73, 142.49, 141.33, 138.93, 136.36, 136.12, 130.51, 129.53, 129.16, 128.53, 127.78, 126.60, 121.99, 116.23, 114.49, 54.76, 47.68, 45.88, 29.58, 28.80, 22.47, 22.44. HRMS (ESI) m/z calculated for C₂₆H₂₈N₅O [M+H]⁺426.2289, found 426.2290.

### N-(cyanomethyl)-4-(9-methyl-3H-pyrazolo[4,3-f]quinolin-7-yl)benzamide (Compound HSH3202)

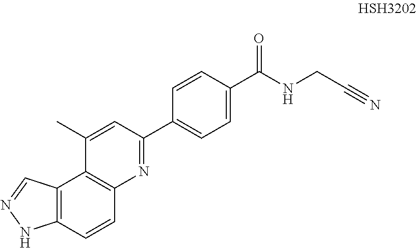 [SMILES: Cc1cc(-c2ccc(C(=O)NCC#N)cc2)nc2ccc3[nH]ncc3c12]

Synthesized using Method F: Off white solid (23 mg, 0.5 mmol, 13.5%). ¹H NMR (800 MHz, (CD₃)₂SO) δ 9.26 (m, 1H), 8.60 (s, 1H), 8.41 (d, J=8.7 Hz, 2H), 8.19 (d, J=9.0 Hz, 1H), 8.05 (d, J=8.7 Hz, 2H), 8.01-7.91 (m, 2H), 4.36 (q, J=5.6 Hz, 2H), 2.97 (d, J=8.4 Hz, 3H). ¹³C NMR (201 MHz, (CD₃)₂SO) δ 166.77, 152.12, 146.15, 144.54, 142.37, 136.05, 133.37, 129.68, 128.27, 127.19, 122.31, 120.83, 118.02, 116.80, 115.64, 28.19, 22.71. HRMS (ESI) m/z calculated for C₂₀H₁₆NO [M+H]⁺342.1349, found 342.1353.

### N-(cyanomethyl)-3-fluoro-4-(8,9,10,11-tetrahydro-3H-pyrazolo[4,3-a]phenanthridin-7-yl)benzamide (Compound HSH3203)

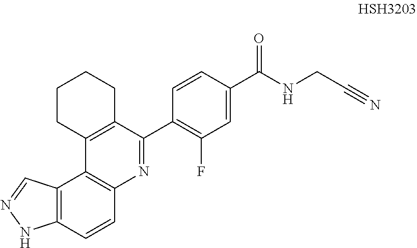 [SMILES: N#CCNC(=O)c1ccc(-c2nc3ccc4[nH]ncc4c3c3c2CCCC3)c(F)c1]

Synthesized using Method F: Off white solid (70 mg, 0.5 mmol, 35%). ¹H NMR (500 MHZ, (CD₃)₂SO) δ 8.59 (s, 1H), 7.88 (d, J=9.1 Hz, 1H), 7.81 (d, J=9.1 Hz, 1H), 7.51 (t, J=7.6 Hz, 1H), 7.35 (m, 2H), 3.35 (t, J=6.5 Hz, 2H), 2.64 (t, J=6.3 Hz, 2H), 2.52 (s, 2H), 2.04-1.96 (m, 2H), 1.79 (dd, J=7.5, 4.2 Hz, 2H). ¹³C NMR (126 MHZ, (CD₃)₂SO) δ 169.03, 158.32 (1J=245.7), 151.69, 143.97, 142.42, 140.00, 139.02 (3J=7.56 Hz), 134.97, 131.90, 130.08, 129.76 (2J=16.4 Hz), 129.44, 123.46, 122.44, 116.21, 115.64, 114.58 (2J=23.9 Hz), 34.73, 29.47, 27.38, 22.59, 22.19.

### (2-fluoro-4-(8,9,10,11-tetrahydro-3H-pyrazolo[4,3-a]phenanthridin-7-yl)phenyl) (4-methylpiperazin-1-yl) methanone (Compound HSH3204)

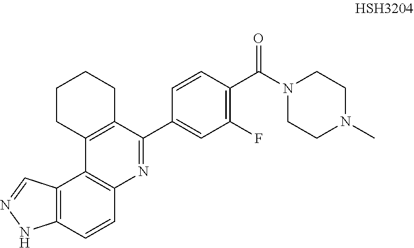 [SMILES: CN1CCN(C(=O)c2ccc(-c3nc4ccc5[nH]ncc5c4c4c3CCCC4)cc2F)CC1]

Synthesized using Method F: Yellow solid (18 mg, 0.25 mmol, 8%). ¹H NMR (500 MHZ, (CD₃)₂SO) δ 8.57 (s, 1H), 7.86 (d, J=9.0 Hz, 1H), 7.81 (s, 1H), 7.47 (t, J=5.1 Hz, 3H), 3.68 (s, 2H), 2.81 (t, J=6.4 Hz, 2H), 2.42-2.27 (m, 5H), 2.22 (d, J=7.7 Hz, 4H), 2.01 (m, 2H), 1.82-1.71 (m, 2H). ¹³C NMR (126 MHZ, (CD₃)₂SO) δ 164.36, 161.15, 158.69 (d, 1J=247 Hz), 154.77, 144.56 (d, 3J-7.56 Hz), 143.85, 142.72, 142.45, 129.56, 129.22, 128.81, 126.08, 123.87 (d, 2]=17.6 Hz), 122.30, 121.84, 116.75 (d, 2J=21 Hz), 116.22, 55.25, 46.10, 45.98, 29.66, 28.68, 22.54, 22.48. HRMS (ESI) m/z calculated for C₂₆H₂₇FN₅O [M+H]⁺444.2194, found 444.2194.

### (3-fluoro-4-(8,9,10,11-tetrahydro-3H-pyrazolo[4,3-a]phenanthridin-7-yl)phenyl) (4-methylpiperazin-1-yl) methanone (Compound HSH3205)

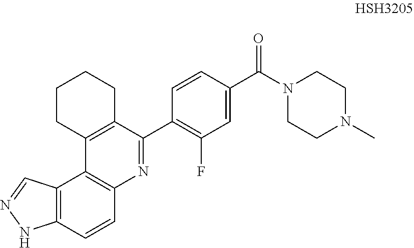 [SMILES: CN1CCN(C(=O)c2ccc(-c3nc4ccc5[nH]ncc5c4c4c3CCCC4)c(F)c2)CC1]

Synthesized using Method F: Off white solid (56 mg, 0.5 mmol, 25.3%). ¹H NMR (500 MHz, (CD₃)₂SO) δ 8.58 (s, 1H), 7.88 (d, J=9.1 Hz, 1H), 7.82 (s, 1H), 7.52 (t, J=7.6 Hz, 1H), 7.38-7.29 (m, 2H), 3.56 (s, 4H), 3.41-3.30 (m, 2H), 3.27-3.11 (m, 2H), 2.65 (t, J=6.2 Hz, 2H), 2.46 (s, 2H), 2.28 (s, 3H), 2.00 (p, J=6.5 Hz, 2H), 1.80 (tq, J=9.2, 6.2, 4.4 Hz, 2H). 13C NMR (126 MHZ, (CD₃)₂SO) δ 167.89, 160.36 (d, 1J=245.7 Hz), 158.41, 151.64, 143.87, 142.44, 138.89, 138.35 (d, 3J=7.56 Hz), 136.34, 132.08, 130.11, 129.98, 129.44, 123.42, 122.42, 116.44, 114.61 (d, 2J=23 Hz), 54.65, 54.55, 45.62, 29.47, 27.40, 22.59, 22.19. HRMS (ESI) m/z calculated for C₂₆H₂₇F₅O [M+H]⁺444.2194, found 444.2193.

### (2-methoxy-4-(8,9,10,11-tetrahydro-3H-pyrazolo[4,3-a]phenanthridin-7-yl)phenyl) (4-methylpiperazin-1-yl) methanone (Compound HSH3206)

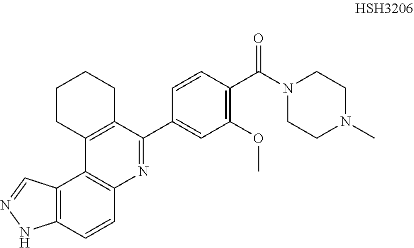 [SMILES: COc1cc(-c2nc3ccc4[nH]ncc4c3c3c2CCCC3)ccc1C(=O)N1CCN(C)CC1]

Synthesized using Method F: Off white solid (44 mg, 0.5 mmol, 19.31%). ¹H NMR (800 MHz, (CD₃)₂SO) δ 8.61-8.53 (m, 1H), 7.87 (dd, J=9.0, 4.2 Hz, 1H), 7.84 (s, 1H), 7.26 (dd, J=7.6, 4.2 Hz, 1H), 7.22 (d, J=4.1 Hz, 1H), 7.15 (dd, J=7.9, 3.8 Hz, 1H), 3.84 (d, J=4.4 Hz, 3H), 3.72-3.64 (m, 1H), 3.61 (d, J=16.7 Hz, 1H), 3.33-3.28 (m, 2H), 3.25-3.17 (m, 2H), 2.80 (q, J=6.1, 5.6 Hz, 2H), 2.34 (m, 3H), 2.26-2.22 (m, 1H), 2.21 (d, J=4.3 Hz, 3H), 2.01 (m, 2H), 1.76 (m, J=6.1 Hz, 2H). ¹³C NMR (201 MHz, (CD₃)₂SO) δ 166.75, 156.28, 155.04, 143.49, 143.38, 142.43, 138.71, 136.29, 129.52, 129.26, 127.66, 125.47, 121.98, 121.76, 116.47, 114.47, 112.59, 56.11, 55.13, 54.68, 46.61, 45.98, 41.34, 29.57, 28.71, 22.47, 22.42. HRMS (ESI) m/z calculated for C₂₇H₃₀N₅O₂ [M+H]⁺456.2394, found 456.2394.

### N-(1-(dimethylamino) propan-2-yl)-4-(8,9,10,11-tetrahydro-3H-pyrazolo[4,3-a]phenanthridin-7-yl)benzamide (Compound 3207)

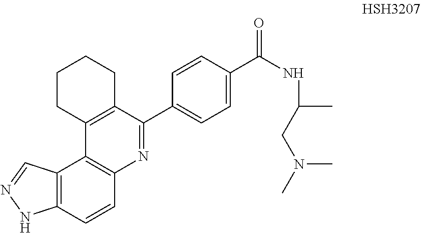 [SMILES: CC(CN(C)C)NC(=O)c1ccc(-c2nc3ccc4[nH]ncc4c3c3c2CCCC3)cc1]

Synthesized using Method F: Off white solid (31 mg, 0.5 mmol, 14.5%). ¹H NMR (800 MHz, (CD₃)₂SO) δ 8.57 (d, J=8.7 Hz, 1H), 8.08 (d, J=8.3 Hz, 1H), 7.94 (d, J=7.9 Hz, 2H), 7.88-7.80 (m, 2H), 7.62 (d, J=8.0 Hz, 2H), 4.22-4.15 (m, 1H), 2.76 (t, J=6.2 Hz, 2H), 2.51-2.47 (m, 2H), 2.47 (dd, J=12.2, 7.4 Hz, 1H), 2.28 (dd, J=12.2, 6.7 Hz, 1H), 2.20 (d, J=5.4 Hz, 6H), 2.03-1.99 (m, 2H), 1.79-1.73 (m, 2H), 1.18 (d, J=6.7 Hz, 3H). ¹³C NMR (126 MHZ, (CD₃)₂SO) & 165.91, 156.21, 143.89, 142.49, 139.11, 136.26, 134.66, 129.66, 129.31, 129.22, 127.35, 127.09, 122.10, 116.31, 114.63, 64.90, 45.93, 43.77, 29.64, 28.82, 22.59, 22.53, 19.49. HRMS (ESI) m/z calculated for C₂₆H₃₀NO [M+H]⁺428.2444, found 428.2445.

### (4-(2-hydroxyethyl) piperazin-1-yl) (4-(8,9,10,11-tetrahydro-3H-pyrazolo[4,3-a]phenanthridin-7-yl)phenyl) methanone (Compound HSH3208)

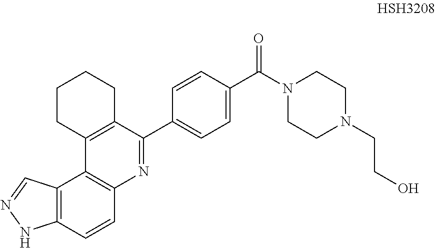 [SMILES: O=C(c1ccc(-c2nc3ccc4[nH]ncc4c3c3c2CCCC3)cc1)N1CCN(CCO)CC1]

Synthesized using Method F: Off white solid (27 mg, 0.75 mmol, 7.9%). ¹H NMR (500 MHz, (CD₃)₂SO) δ 8.56 (s, 1H), 7.87-7.75 (m, 2H), 7.61 (d, J=8.1 Hz, 2H), 7.47 (d, J=8.1 Hz, 2H), 4.28 (t, J=5.3 Hz, 1H), 3.63-3.42 (m, 6H), 3.34 (dq, J=10.4, 5.0 Hz, 2H), 3.20 (d, J=14.4 Hz, 2H), 2.79 (t, J=6.2 Hz, 2H), 2.48-2.42 (m, 4H), 2.06-1.95 (m, 2H), 1.80-1.71 (m, 2H). ¹³C NMR (126 MHZ, (CD₃)₂SO) δ 169.30, 156.16, 143.81, 142.52, 142.44, 138.86, 136.38, 135.82, 129.62, 129.53, 129.27, 127.03, 122.06, 116.50, 114.36, 60.52, 59.02, 53.58, 29.66, 28.85, 22.61, 22.53.

### (4-methylpiperazin-1-yl) (4-(3,8,10,11-tetrahydropyrano[3,4-c]pyrazolo[4,3-f]quinolin-7-yl)phenyl) methanone (Compound HSH3209)

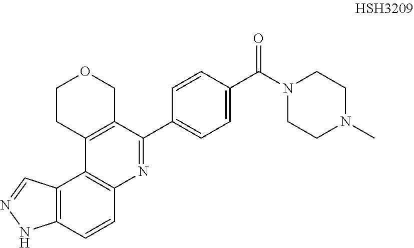 [SMILES: CN1CCN(C(=O)c2ccc(-c3nc4ccc5[nH]ncc5c4c4c3COCC4)cc2)CC1]

Synthesized using Method F: Off white solid (5 mg, 0.5 mmol, 4.7%). ¹H NMR (800 MHZ, (CD₃)₂SO) δ 8.60 (s, 1H), 7.92 (d, J=9.0 Hz, 1H), 7.86 (d, J=9.3 Hz, 1H), 7.69-7.62 (m, 2H), 7.53-7.48 (m, 2H), 4.81 (s, 2H), 4.16 (t, J=5.9 Hz, 2H), 3.65 (s, 2H), 3.40 (t, J=6.1 Hz, 4H), 2.47-2.28 (m, 4H), 2.24 (s, 3H). ¹³C NMR (201 MHZ, (CD₃)₂SO) δ 169.13, 152.89, 144.33, 140.77, 139.40, 138.84, 136.15, 135.67, 129.41, 129.25, 127.42, 127.20, 121.39, 116.04, 115.07, 66.80, 64.35, 54.79, 47.28, 45.81, 41.78, 40.49, 28.62. HRMS (ESI) m/z calculated for C₂₅H₂₆N₅O₂ [M+H]⁺428.2081, found 428.2080.

### N-(1-methylpiperidin-4-yl)-4-(8,9,10,11-tetrahydro-3H-pyrazolo[4,3-a]phenanthridin-7-yl)benzamide (Compound HSH3210)

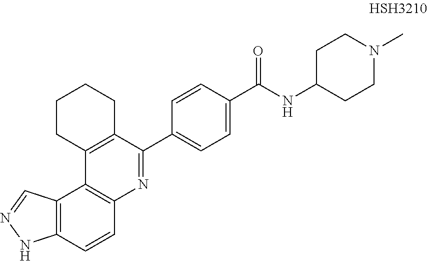 [SMILES: CN1CCC(NC(=O)c2ccc(-c3nc4ccc5[nH]ncc5c4c4c3CCCC4)cc2)CC1]

Synthesized using Method F: Off white solid (83 mg, 0.75 mmol, 25%). ¹H NMR (500 MHz, (CD₃)₂SO) δ 8.56 (s, 1H), 8.20 (d, J=7.6 Hz, 1H), 7.95 (d, J=8.1 Hz, 2H), 7.88-7.75 (m, 2H), 7.61 (d, J=8.1 Hz, 2H), 3.87-3.70 (m, 1H), 3.32 (t, J=6.6 Hz, 2H), 2.85-2.69 (m, 4H), 2.19 (s, 3H), 2.08-1.95 (m, 4H), 1.84-1.59 (m, 6H).

### (4-(9,9-dioxido-3,8,10,11-tetrahydropyrazolo[4,3-f]thiopyrano[3,4-c]quinolin-7-yl)phenyl) (4-methylpiperazin-1-yl) methanone (Compound HSH3211)

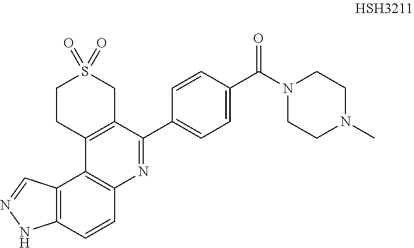 [SMILES: CN1CCN(C(=O)c2ccc(-c3nc4ccc5[nH]ncc5c4c4c3CS(=O)(=O)CC4)cc2)CC1]

Synthesized using Method F: Off white solid (53 mg, 0.75 mmol, 14.9%). ¹H NMR (800 MHz, (CD₃)₂SO) δ 8.70 (s, 1H), 8.02 (d, J=9.0 Hz, 1H), 7.91-7.84 (m, 1H), 7.68 (d, J=8.0 Hz, 2H), 7.63 (d, J=8.1 Hz, 2H), 4.58 (s, 2H), 4.02 (d, J=6.8 Hz, 2H), 3.72 (t, J=6.7 Hz, 2H), 3.38 (d, J=35.2 Hz, 8H), 2.78 (s, 3H). ¹³C NMR (201 MHZ, (CD₃)₂SO) δ 169.34, 155.40, 151.42, 144.71, 140.93, 138.97, 136.64, 135.23, 129.78, 129.29, 129.16, 127.56, 122.30, 121.13, 115.67, 52.47, 51.65, 45.95, 42.57, 40.40, 30.51. HRMS (ESI) m/z calculated for C₂₅H₂₆N₅OS [M+H]⁺476.1751, found 476.1750.

### N-(2-(dimethylamino)ethyl)-4-(9,9-dioxido-3,8,10,11-tetrahydropyrazolo[4,3-f]thiopyrano[3,4-c]quinolin-7-yl)benzamide (Compound HSH3212)

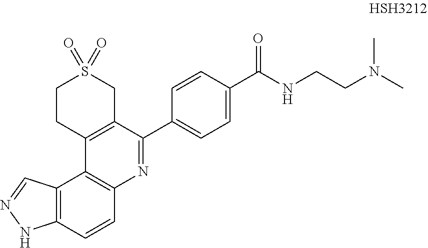 [SMILES: CN(C)CCNC(=O)c1ccc(-c2nc3ccc4[nH]ncc4c3c3c2CS(=O)(=O)CC3)cc1]

Synthesized using Method F: Off white solid (68 mg, 0.75 mmol, 19.6%). ¹H NMR (800 MHz, (CD₃)₂SO) δ 8.71 (s, 1H), 8.50 (t, J=4.8 Hz, 1H), 8.01 (dt, J=9.3, 4.2 Hz, 3H), 7.92-7.84 (m, 1H), 7.71-7.61 (m, 2H), 4.51 (s, 2H), 4.01 (d, J=9.4 Hz, 2H), 3.71 (d, J=9.0 Hz, 2H), 3.42 (p, J=6.9 Hz, 2H), 2.48-2.40 (m, 2H), 2.21 (d, J=3.9 Hz, 6H). ¹³C NMR (201 MHZ, (CD₃)₂SO) & 166.13, 155.51, 144.76, 141.95, 139.88, 138.96, 135.59, 134.85, 129.54, 129.37, 127.64, 122.25, 121.13, 117.19, 115.61, 58.58, 51.72, 45.93, 45.66, 37.90, 30.48. HRMS (ESI) m/z calculated for C₂₄H₂₆N503S [M+H]⁺464.1750, found 464.1751.

### N-(2-(pyrrolidin-1-yl)ethyl)-4-(3,8,10,11-tetrahydropyrano[3,4-c]pyrazolo[4,3-f]quinolin-7-yl)benzamide (Compound HSH3214)

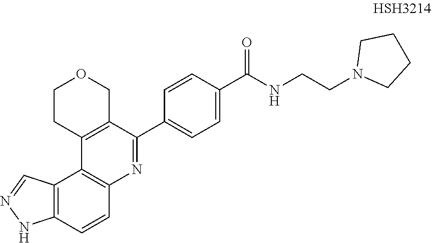 [SMILES: O=C(NCCN1CCCC1)c1ccc(-c2nc3ccc4[nH]ncc4c3c3c2COCC3)cc1]

Synthesized using Method F: Off white solid (99 mg, 0.75 mmol, 29.9%). ¹H NMR (500 MHz, (CD₃)₂SO) δ 8.57 (s, 1H), 8.38 (t, J=5.7 Hz, 1H), 8.03-7.93 (m, 2H), 7.90 (d, J=9.2 Hz, 1H), 7.84 (d, J=9.1 Hz, 1H), 7.71-7.61 (m, 2H), 4.78 (s, 2H), 4.14 (t, J=5.9 Hz, 2H), 3.44 (t, J=6.3 Hz, 2H), 3.39 (q, J=6.9, 6.0 Hz, 2H), 2.63 (t, J=6.9 Hz, 2H), 2.58-2.48 (m, 4H), 1.68 (h, J=3.2 Hz, 4H). ¹³C NMR (126 MHZ, (CD₃)₂SO) δ 166.35, 153.00, 144.49, 142.39, 139.93, 139.45, 134.92, 134.66, 134.53, 129.53, 129.17, 127.54, 127.47, 121.52, 116.09, 66.86, 64.42, 55.36, 54.10, 39.23, 28.73, 23.74. HRMS (ESI) m/z calculated for C₂₆H₂₈N₅O₂ [M+H]⁺442.2237, found 442.2238.

### piperazin-1-yl(4-(8,9,10,11-tetrahydro-3H-pyrazolo[4,3-a]phenanthridin-7-yl)phenyl) methanone (Compound HSH3215)

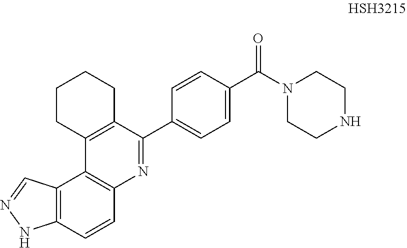 [SMILES: O=C(c1ccc(-c2nc3ccc4[nH]ncc4c3c3c2CCCC3)cc1)N1CCNCC1]

Synthesized using Method G: Off white solid (37 mg, 0.26 mmol, 34.6%). ¹H NMR (800 MHz, (CD₃)₂SO) δ 8.59 (s, 1H), 7.87 (d, J=9.0 Hz, 1H), 7.82 (d, J=9.0 Hz, 1H), 7.63 (d, J=8.0 Hz, 2H), 7.50 (d, J=7.9 Hz, 2H), 3.77-3.51 (m, 4H), 3.33 (d, J=6.7 Hz, 2H), 2.86 (m, 4H), 2.79 (t, J=6.2 Hz, 2H), 2.01 (m, 2H), 1.76 (m, 2H). HRMS (ESI) m/z calculated for C₂₅H₂₆N₅O [M+H]⁺412.2132, found 412.2133.

### N-(2-(dimethylamino)ethyl)-5-(8,9,10,11-tetrahydro-3H-pyrazolo[4,3-a]phenanthridin-7-yl)thiophene-2-carboxamide (Compound HSH3216)

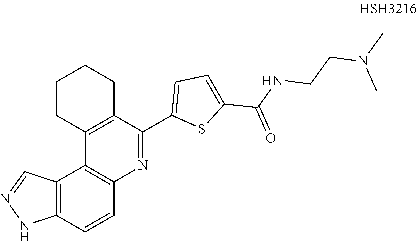 [SMILES: CN(C)CCNC(=O)c1ccc(-c2nc3ccc4[nH]ncc4c3c3c2CCCC3)s1]

Synthesized using Method F: Off white solid (35 mg, 0.5 mmol, 16.7%). ¹H NMR (500 MHz, (CD₃)₂SO) δ 8.54 (s, 1H), 8.28 (t, J=5.8 Hz, 1H), 7.85 (d, J=9.1 Hz, 1H), 7.77 (d, J=9.1 Hz, 1H), 7.72 (d, J=4.1 Hz, 1H), 7.57 (d, J=4.1 Hz, 1H), 3.36 (q, J=6.8 Hz, 2H), 3.30 (s, 2H), 3.08 (t, J=6.1 Hz, 2H), 2.43 (t, J=6.8 Hz, 2H), 2.19 (s, 6H), 2.00 (m, 2H), 1.90-1.81 (m, 2H). ¹³C NMR (201 MHz, (CD₃)₂SO) δ 161.63, 148.93, 148.44, 143.45, 143.24, 140.75, 138.98, 136.37, 129.23, 128.96, 128.80, 128.30, 122.04, 116.06, 115.02, 58.44, 45.41, 37.53, 29.89, 28.90, 22.43, 22.17. HRMS (ESI) m/z calculated for C₂₃H₂₆N₅OS [M+H]⁺420.1852, found 420.1850.

### N-(2-(dimethylamino)ethyl)-3-fluoro-4-(8,9,10,11-tetrahydro-3H-pyrazolo[4,3-a]phenanthridin-7-yl)benzamide (Compound HSH3217)

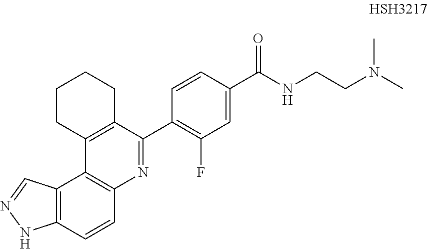 [SMILES: CN(C)CCNC(=O)c1ccc(-c2nc3ccc4[nH]ncc4c3c3c2CCCC3)c(F)c1]

Synthesized using Method F: Off white solid (53 mg, 0.75 mmol, 16.4%). ¹H NMR (800 MHZ, (CD₃)₂SO) δ 8.61 (s, 1H), 8.53 (t, J=5.6 Hz, 1H), 7.89 (d, J=9.0 Hz, 1H), 7.83 (dd, J=7.8, 1.8 Hz, 2H), 7.79 (m, 1H), 7.57 (t, J=7.6 Hz, 1H), 3.44 (q, J=6.7 Hz, 3H), 3.36 (t, J=6.8 Hz, 2H), 2.63 (t, J=6.4 Hz, 2H), 2.54 (t, J=6.8 Hz, 2H), 2.28 (s, 6H), 2.02 (q, J=6.2, 5.0 Hz, 2H), 1.84-1.77 (m, 2H). ¹³C NMR (201 MHZ, (CD₃)₂SO) δ 165.08, 160.01 (d, 1J=245 Hz), 151.56, 143.82, 142.39, 137.00, 136.96, 131.99, 131.78, 131.76, 131.48, 131.39, 129.93, 129.40, 123.66, 122.38, 116.31, 114.76 (d, 2J-24 Hz), 58.33, 45.38, 37.76, 29.39, 27.28, 22.48, 22.08. HRMS (ESI) m/z calculated for C₂₅H₂₇FN₅O [M+H]⁺432.2194, found 432.2190.

### 3-fluoro-N-(2-(pyrrolidin-1-yl)ethyl)-4-(8,9,10,11-tetrahydro-3H-pyrazolo[4,3-a]phenanthridin-7-yl)benzamide (Compound HSH3218)

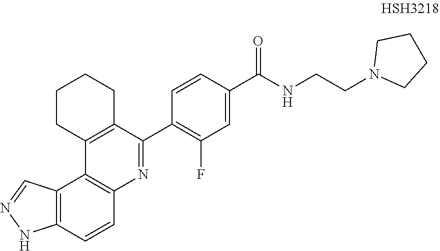 [SMILES: O=C(NCCN1CCCC1)c1ccc(-c2nc3ccc4[nH]ncc4c3c3c2CCCC3)c(F)c1]

Synthesized using Method F: Off white solid (146 mg, 0.75 mmol, 42.5%). ¹H NMR (500 MHz, (CD₃)₂SO) δ 8.59 (s, 1H), 8.46 (t, J=5.7 Hz, 1H), 7.87 (d, J=9.1 Hz, 1H), 7.81 (m, 2H), 7.77 (dd, J=10.7, 1.8 Hz, 1H), 7.54 (t, J=7.6 Hz, 1H), 3.46-3.38 (m, 2H), 3.34 (t, J=6.5 Hz, 2H), 2.62 (dd, J=8.3, 5.6 Hz, 4H), 2.51 (m, 4H), 2.05-1.94 (m, 2H), 1.84-1.73 (m, 2H), 1.68 (h, J=3.2 Hz, 4H). ¹³C NMR (126 MHZ, (CD₃)₂SO) δ 165.12, 160.48 (d, 1J=245 Hz), 151.65, 143.96, 142.42, 137.22, 137.17, 131.84, 131.80, 131.57, 131.43, 129.98, 129.48, 123.70, 122.47, 116.23, 115.52, 114.86 (d, 2J=24 Hz), 55.28, 54.08, 39.36, 29.46, 27.36, 23.74, 22.57, 22.17. HRMS (ESI) m/z calculated for C₂₇H₂₉FN₅O [M+H]⁺458.2350, found 458.2350.

### (2-methoxy-4-(8,9,10,11-tetrahydro-3H-naphtho[1,2-e]indazol-7-yl)phenyl) (4-methylpiperazin-1-yl) methanone (Compound HSH3222)

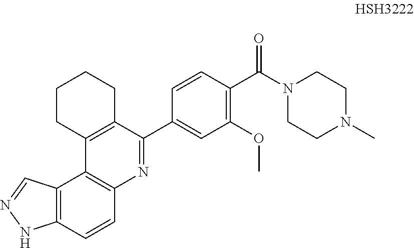 [SMILES: COc1cc(-c2nc3ccc4[nH]ncc4c3c3c2CCCC3)ccc1C(=O)N1CCN(C)CC1]

Synthesized using Method F: Off white solid (77 mg, 0.75 mmol, 22.6%). ¹H NMR (800 MHz, (CD₃)₂SO) δ 8.61-8.53 (m, 1H), 7.87 (dd, J=9.0, 4.2 Hz, 1H), 7.84 (s, 1H), 7.26 (dd, J=7.6, 4.2 Hz, 1H), 7.22 (d, J=4.1 Hz, 1H), 7.15 (dd, J=7.9, 3.8 Hz, 1H), 3.84 (d, J=4.4 Hz, 3H), 3.72-3.64 (m, 1H), 3.61 (d, J=16.7 Hz, 1H), 3.33-3.28 (m, 2H), 3.25-3.17 (m, 2H), 2.80 (q, J=6.1, 5.6 Hz, 2H), 2.34 (m, 3H), 2.26-2.22 (m, 1H), 2.21 (d, J=4.3 Hz, 3H), 2.01 (m, 2H), 1.76 (m, J=6.1 Hz, 2H). ¹³C NMR (201 MHz, (CD₃)₂SO) δ 166.75, 156.28, 155.04, 143.49, 143.38, 142.43, 138.71, 136.29, 129.52, 129.26, 127.66, 125.47, 121.98, 121.76, 116.47, 114.47, 112.59, 56.11, 55.13, 54.68, 46.61, 45.98, 41.34, 29.57, 28.71, 22.47, 22.42. HRMS (ESI) m/z calculated for C₂₇H₃₀N₅O₂ [M+H]⁺456.2394, found 456.2394.

### (4-(9-cyclopropyl-3H-benzo[e]indazol-7-yl)phenyl) (4-methylpiperazin-1-yl) methanone (Compound HSH3223)

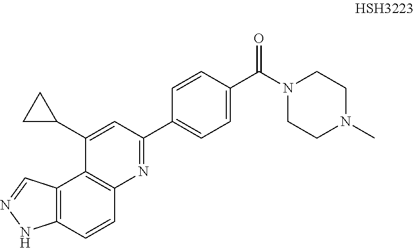 [SMILES: CN1CCN(C(=O)c2ccc(-c3cc(C4CC4)c4c(ccc5[nH]ncc54)n3)cc2)CC1]

Synthesized using Method F: Off white solid (69 mg, 0.75 mmol, 22.4%). ¹H NMR (800 MHz, (CD₃)₂SO) δ 8.84 (s, 1H), 8.32 (d, J=8.1 Hz, 2H), 7.98-7.91 (m, 3H), 7.52 (d, J=8.2 Hz, 2H), 3.63 (s, 2H), 2.65 (s, 1H), 2.45-2.23 (m, 4H), 2.20 (s, 3H), 1.35-1.28 (m, 2H), 1.04 (t, J=5.3 Hz, 2H). ¹³C NMR (201 MHZ, (CD₃)₂SO) δ 169.21, 152.76, 148.76, 146.13, 140.35, 138.60, 136.59, 136.43, 129.70, 127.72, 127.34, 123.08, 116.89, 116.52, 115.63, 54.95, 47.48, 45.90, 41.98, 40.43, 16.55, 7.65. HRMS (ESI) m/z calculated for C₂₅H₂₆N₅O [M+H]⁺412.2132, found 412.2132.

### 7-(4-((4-methylpiperazin-1-yl)methyl)phenyl)-8,9,10,11-tetrahydro-3H-naphtho[1,2-e]indazole (Compound HSH3225)

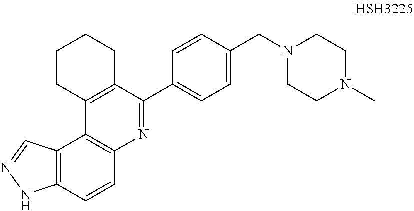 [SMILES: CN1CCN(Cc2ccc(-c3nc4ccc5[nH]ncc5c4c4c3CCCC4)cc2)CC1]

Synthesized using Method H: Off white solid (30 mg, 0.5 mmol, 14.6%). ¹H NMR (500 MHz, (CD₃)₂SO) δ 8.56 (s, 1H), 7.86-7.76 (m, 2H), 7.48 (d, J=8.0 Hz, 2H), 7.37 (d, J=8.0 Hz, 2H), 3.52 (s, 2H), 3.33 (t, J=6.6 Hz, 2H), 2.78 (t, J=6.2 Hz, 2H), 2.42 (t, J=4.9 Hz, 4H), 2.38-2.27 (m, 4H), 2.16 (s, 3H), 2.01 (m, 2H), 1.79-1.70 (m, 2H). ¹³C NMR (201 MHz, (CD₃)₂SO) δ 169.39, 158.54, 158.39, 156.06, 143.74, 142.51, 142.32, 135.48, 129.52, 129.48, 129.20, 127.01, 121.99, 116.94, 116.15, 47.82, 45.28, 42.13, 40.37, 29.57, 28.78, 22.48, 22.40. HRMS (ESI) m/z calculated for C₂₆H₃₀N₅ [M+H]⁺412.2495, found 412.2496

### (4-(methylsulfonyl) piperazin-1-yl) (4-(3,8,10,11-tetrahydroisochromeno[5,6-e]indazol-7-yl)phenyl) methanone (Compound HSH3126)

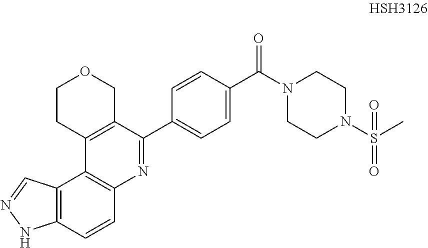 [SMILES: CS(=O)(=O)N1CCN(C(=O)c2ccc(-c3nc4ccc5[nH]ncc5c4c4c3COCC4)cc2)CC1]

Synthesized using Method F: Off white solid (127 mg, 1 mmol, 25.9%). ¹H NMR (500 MHz, (CD₃)₂SO) δ 8.52 (s, 1H), 7.88 (t, J=10.6 Hz, 2H), 7.65 (d, J=8.1 Hz, 2H), 7.54 (d, J=8.1 Hz, 2H), 4.78 (s, 2H), 4.13 (t, J=5.9 Hz, 2H), 3.65 (m, 4H), 3.36 (t, J=5.9 Hz, 2H), 3.25-3.17 (m, 4H), 2.91 (s, 3H). ¹³C NMR (126 MHZ, (CD₃)₂SO) δ 169.50, 152.93, 144.29, 141.17, 139.43, 138.80, 135.91, 135.80, 129.50, 129.38, 127.51, 127.39, 121.39, 116.31, 115.07, 66.87, 64.42, 45.85, 35.09, 28.69.

### N-(2-(dimethylamino)ethyl)-4-(1-methyl-8,9,10,11-tetrahydro-3H-pyrazolo[4,3-a]phenanthridin-7-yl)benzamide (Compound HSH3127)

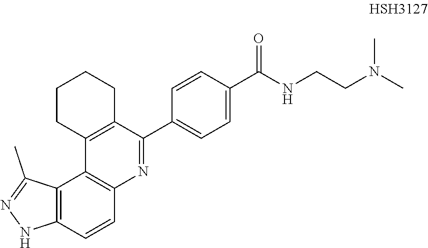 [SMILES: Cc1n[nH]c2ccc3nc(-c4ccc(C(=O)NCCN(C)C)cc4)c4c(c3c12)CCCC4]

Synthesized using Method F: Off white solid (38.4 mg, 0.5 mmol, 17.9%). ¹H NMR (800 MHz, (CD₃)₂SO) δ 8.28 (t, J=5.7 Hz, 1H), 7.97-7.94 (m, 2H), 7.74 (d, J=8.9 Hz, 2H), 7.63-7.60 (m, 2H), 3.45-3.39 (m, 4H), 2.87 (s, 3H), 2.80-2.76 (m, 2H), 2.47 (t, J=6.9 Hz, 2H), 2.22 (s, 6H), 1.80 (h, J=4.7, 3.3 Hz, 4H). ¹³C NMR (201 MHz, (CD₃)₂SO) δ 166.38, 155.59, 144.25, 143.86, 142.87, 142.43, 141.24, 134.32, 129.78, 129.25, 128.25, 127.35, 127.23, 123.83, 114.51, 58.64, 45.61, 40.64, 37.97, 31.47, 27.87, 22.41, 22.08. HRMS (ESI) m/z calculated for C₂₆H₃₀N₅O [M+H]⁺428.2444, found 428.2445.

### 4-(1-methyl-8,9,10,11-tetrahydro-3H-pyrazolo[4,3-a]phenanthridin-7-yl)-N-(2-(pyrrolidin-1-yl)ethyl)benzamide (Compound HSH3128)

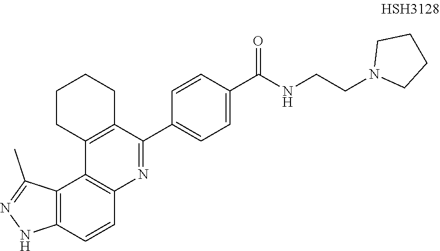 [SMILES: Cc1n[nH]c2ccc3nc(-c4ccc(C(=O)NCCN5CCCC5)cc4)c4c(c3c12)CCCC4]

Synthesized using Method F: Off white solid (40.7 mg, 0.3 mmol, 29.92%). ¹H NMR (800 MHz, (CD₃)₂SO) δ 8.33 (t, J=5.6 Hz, 1H), 7.98-7.94 (m, 2H), 7.76-7.67 (m, 2H), 7.64-7.59 (m, 2H), 3.48-3.39 (m, 4H), 2.87 (s, 3H), 2.78 (t, J=6.0 Hz, 2H), 2.64 (t, J=7.0 Hz, 2H), 2.57-2.51 (m, J=2.8, 2.0 Hz, 4H), 1.84-1.77 (m, 4H), 1.70 (h, J=3.1 Hz, 4H). ¹³C NMR (201 MHZ, (CD₃)₂SO) δ 166.36, 155.59, 144.35, 144.28, 143.85, 142.43, 134.34, 129.77, 129.24, 128.25, 127.35, 127.24, 127.08, 123.83, 114.27, 55.30, 54.01, 40.65, 39.18, 31.47, 27.87, 23.68, 22.41, 22.08. HRMS (ESI) m/z calculated for C₂₈H₃₂N₅O [M+H]⁺454.2601, found 454.2603.

### (4-(9-methyl-3H-pyrazolo[4,3-f]quinolin-7-yl)phenyl) (4-methylpiperazin-1-yl) methanone

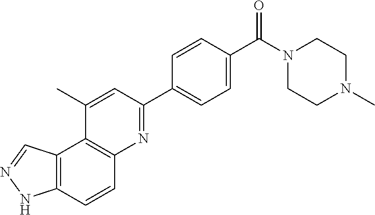 [SMILES: Cc1cc(-c2ccc(C(=O)N3CCN(C)CC3)cc2)nc2ccc3[nH]ncc3c12]

Synthesized using Method D: Off white solid (53 mg, 0.5 mmol, 27.5%) ¹H NMR (800 MHz, (CD₃)₂SO) δ 8.60 (d, J=6.8 Hz, 1H), 8.34 (m, 2H), 8.16 (d, J=6.9 Hz, 1H), 7.96 (dt, J=31.9, 8.0 Hz, 2H), 7.54 (d, J=7.8 Hz, 2H), 3.56-3.41 (m, 3H), 2.97 (d, J=6.8 Hz, 4H), 2.35 (s, 4H), 2.22 (d, J=6.9 Hz, 3H), 1.67 (d, J=6.2 Hz, 1H). ¹³C NMR (201 MHZ, (CD₃)₂SO) δ 175.1, 169.2, 152.5, 146.3, 144.5, 140.2, 136.7, 136.1, 129.6, 128.0, 127.8, 127.2, 122.2, 120.6, 116.7, 54.9, 46.0, 22.7. HRMS (ESI) m/z calculated for C₂₃H₂₅N₅O [M+H]⁺386.1975, found 386.1974.

### (4-methylpiperazin-1-yl) (4-(3,8,9,10-tetrahydrocyclopenta[c]pyrazolo[4,3-f]quinolin-7-yl)phenyl) methanone

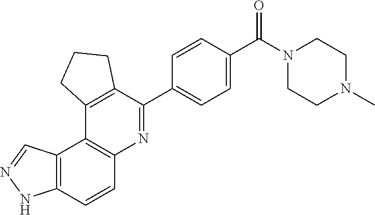 [SMILES: CN1CCN(C(=O)c2ccc(-c3nc4ccc5[nH]ncc5c4c4c3CCC4)cc2)CC1]

Synthesized using Method D: Off white solid (71 mg, 0.5 mmol, 34.5%) ¹H NMR (800 MHz, (CD₃)₂SO) δ 8.48 (s, 1H), 7.97 (d, J=7.8 Hz, 2H), 7.93 (s, 1H), 7.88 (d, J=8.9 Hz, 1H), 7.53 (d, J=7.7 Hz, 2H), 3.52 (s, 2H), 3.31 (d, J=7.4 Hz, 2H), 2.33-2.26 (m, 3H), 2.23 (s, 4H), 1.66 (s, 2H). ¹³C NMR (201 MHz, (CD₃)₂SO) δ 169.2, 151.3, 149.7, 148.9, 148.5, 141.5, 136.0, 135.2, 129.2, 128.9, 128.1, 127.3, 121.0, 119.4, 116.7, 114.8, 54.9, 46.0, 42.5, 33.5, 33.1, 24.9. HRMS (ESI) m/z calculated for C₂₅H₂₇N₅O [M+H]⁺412.2132, found 412.2133.

### (4-(1-methyl-8,9,10,11-tetrahydro-3H-pyrazolo[4,3-a]phenanthridin-7-yl)phenyl) (4-methylpiperazin-1-yl) methanone

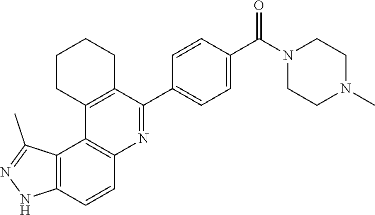 [SMILES: Cc1n[nH]c2ccc3nc(-c4ccc(C(=O)N5CCN(C)CC5)cc4)c4c(c3c12)CCCC4]

Synthesized using Method D: Off white solid (43 mg, 0.5 mmol, 19.5%). ¹H NMR (500 MHz, (CD₃)₂SO) δ 7.75-7.64 (m, 2H), 7.58 (d, J=8.1 Hz, 2H), 7.46 (d, J=8.1 Hz, 2H), 3.38 (dt, J=15.1, 5.2 Hz, 3H), 2.84 (s, 3H), 2.77 (t, J=6.3 Hz, 2H), 2.41-2.31 (m, 5H), 2.21 (s, 5H), 1.82-1.73 (m, 5H). ¹³C NMR (126 MHZ, (CD₃)₂SO) δ 171.8, 169.4, 161.2, 155.6, 142.5, 136.8, 135.7, 131.9, 129.8, 129.5, 128.3, 127.9, 127.2, 127.1, 123.9, 55.0, 54.4, 46.0, 45.1, 31.6, 28.0, 22.5, 22.2. HRMS (ESI) m/z calculated for C₂₇H₃₀N₅O [M+H]⁺440.2444, found 440.2445.

### N-((1H-imidazol-4-yl)methyl)-4-(8,9,10,11-tetrahydro-3/I-pyrazolo[4,3-a]phenanthridin-7-yl)benzamide

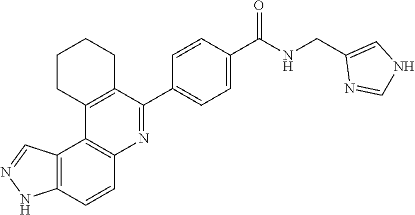 [SMILES: O=C(NCc1c[nH]cn1)c1ccc(-c2nc3ccc4[nH]ncc4c3c3c2CCCC3)cc1]

Synthesized using Method D: Off white solid (111 mg, 0.5 mmol, 52.6%) ¹H NMR (800 MHz, (CD₃)₂SO) δ 8.99 (t, J=7.1 Hz, 1H), 8.05-8.01 (m, 2H), 7.89 (d, J=8.9 Hz, 1H), 7.83 (d, J=6.0 Hz, 2H), 7.65 (d, J=7.9 Hz, 2H), 7.05 (s, 1H), 4.47 (d, J=5.5 Hz, 2H), 2.78 (t, J=6.3 Hz, 2H), 2.02 (p, J=6.3 Hz, 2H), 1.76 (p, J=5.8 Hz, 2H). ¹³C NMR (201 MHZ, (CD₃)₂SO) δ 166.3, 156.1, 150.6, 143.9, 142.5, 140.1, 135.0, 134.0, 129.5, 129.3, 128.8, 127.5, 122.0, 120.6, 117.1, 36.2, 29.6, 28.8, 22.5, 22.4. HRMS (ESI) m/z calculated for C₂₅H₂₃N₆O [M+H]⁺423.1927, found 423.1927.

### N-(2-(1H-imidazol-1-yl)ethyl)-4-(8,9,10,11-tetrahydro-3H-pyrazolo[4,3-a]phenanthridin-7-yl)benzamide

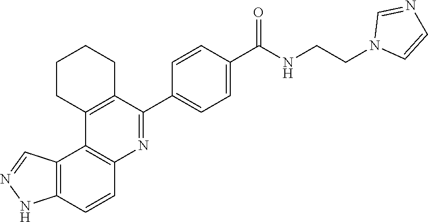 [SMILES: O=C(NCCn1ccnc1)c1ccc(-c2nc3ccc4[nH]ncc4c3c3c2CCCC3)cc1]

Synthesized using Method D: Off white solid (84 mg, 0.5 mmol, 38.5%) ¹H NMR (800 MHz, (CD₃)₂SO) δ 8.74 (t, J=5.5 Hz, 1H), 8.57 (s, 1H), 7.93 (d, J=7.8 Hz, 2H), 7.89 (d, J=8.9 Hz, 1H), 7.86-7.80 (m, 1H), 7.65 (d, J=7.8 Hz, 2H), 7.63 (s, 1H), 7.18 (s, 1H), 6.89 (s, 1H), 4.22 (t, J=6.2 Hz, 2H), 3.63 (q, J=5.9 Hz, 2H), 2.78 (t, J=6.2 Hz, 2H), 2.06-1.99 (m, 2H), 1.80-1.73 (m, 2H). ¹³C NMR (201 MHz, (CD₃)₂SO) & 166.8, 156.1, 145.0, 144.1, 142.5, 138.8, 137.8, 136.3, 133.9, 129.5, 129.4, 128.7, 127.3, 126.9, 120.0, 118.4, 116.4, 114.5, 45.6, 40.9, 29.6, 28.8, 22.5, 22.4. HRMS (ESI) m/z calculated for C₂₆H₂₅N₆O [M+H]⁺437.2084, found 437.2081.

### N-((1H-imidazol-2-yl)methyl)-4-(8,9,10,11-tetrahydro-3//-pyrazolo[4,3-a]phenanthridin-7-yl)benzamide

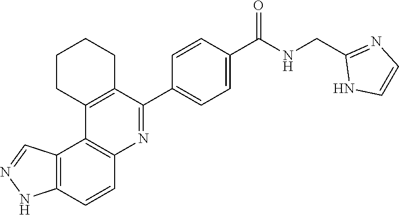 [SMILES: O=C(NCc1ncc[nH]1)c1ccc(-c2nc3ccc4[nH]ncc4c3c3c2CCCC3)cc1]

Synthesized using Method D: Off white solid (65 mg, 0.5 mmol, 30.8%). ¹H NMR (800 MHz, (CD₃)₂SO) δ 9.17 (t, J=5.9 Hz, 1H), 8.57 (s, 1H), 8.07 (d, J=7.8 Hz, 2H), 7.89 (d, J=9.0 Hz, 1H), 7.82 (d, J=9.3 Hz, 1H), 7.65 (d, J=7.8 Hz, 2H), 6.93 (s, 2H), 4.56 (d, J=5.6 Hz, 2H), 2.77 (t, J=6.3 Hz, 2H), 2.00 (q, J=6.8, 6.3 Hz, 2H), 1.75 (p, J=5.6 Hz, 2H). ¹³C NMR (201 MHz, (CD₃)₂SO) & 166.5, 156.0, 146.7, 145.5, 144.1, 143.7, 142.5, 138.8, 136.2, 133.8, 129.5, 129.3, 129.2, 127.6, 127.5, 122.0, 119.1, 116.2, 114.6, 37.5, 29.6, 28.8, 22.5, 22.4. HRMS (ESI) m/z calculated for C₂₅H₂₃N₆O [M+H]⁺423.1927, found 423.1926.

### N-((1-methyl-1H-imidazol-2-yl)methyl)-4-(8,9,10,11-tetrahydro-3H-pyrazolo[4,3-a]phenanthridin-7-yl)benzamide

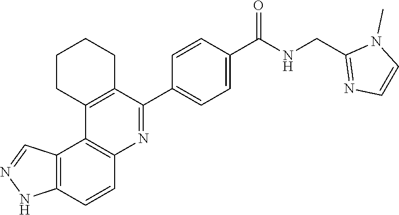 [SMILES: Cn1ccnc1CNC(=O)c1ccc(-c2nc3ccc4[nH]ncc4c3c3c2CCCC3)cc1]

Synthesized using Method D: Off white solid (68 mg, 0.5 mmol, 31.2%). ¹H NMR (500 MHz, (CD₃)₂SO) δ 8.56 (s, 1H), 8.37 (d, J=4.3 Hz, 3H), 8.16 (d, J=8.3 Hz, 3H), 7.04 (d, J=13.1 Hz, 1H), 6.78 (d, J=18.9 Hz, 1H), 4.56 (d, J=5.3 Hz, 2H), 3.68 (s, 5H), 2.77 (t, J=6.2 Hz, 3H), 1.88 (dp, J=127.7, 5.7 Hz, 6H), 1.23 (dd, J=14.7, 7.0 Hz, 2H). ¹³C NMR (126 MHZ, (CD₃)₂SO) δ 166.5, 156.1, 147.2, 144.2, 142.5, 139.9, 134.9, 133.8, 129.5, 129.4, 129.2, 127.7, 127.6, 126.9, 125.7, 122.2, 120.4, 119.2, 36.0, 32.9, 29.7, 28.8, 22.6, 22.5. HRMS (ESI) m/z calculated for C₂₆H₂₆N₆O [M+H]⁺437.2084, found 437.2081.

### N-((4HI-1,2,4-triazol-3-yl)methyl)-4-(8,9,10,11-tetrahydro-3/I-pyrazolo[4,3-a]phenanthridin-7-yl)benzamide

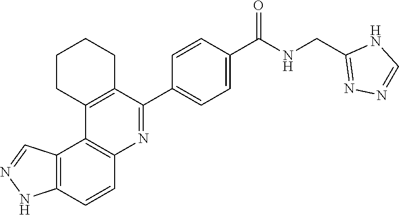 [SMILES: O=C(NCc1nnc[nH]1)c1ccc(-c2nc3ccc4[nH]ncc4c3c3c2CCCC3)cc1]

Synthesized using Method D: Off white solid (79 mg, 0.5 mmol, 46.0%). ¹H NMR (500 MHZ, (CD₃)₂SO) δ 8.58 (s, 1H), 8.43-8.06 (m, 2H), 8.01 (d, J=7.8 Hz, 2H), 7.84 (q, J=9.1 Hz, 2H), 7.65 (d, J=7.8 Hz, 2H), 4.61 (d, J=5.6 Hz, 2H), 3.34 (d, J=13.1 Hz, 2H), 2.78 (t, J=6.1 Hz, 2H), 1.89 (dp, J=125.7, 6.1 Hz, 4H). ¹³C NMR (126 MHZ, (CD₃)₂SO) δ 166.7, 161.5, 156.2, 148.6, 144.2, 143.9, 142.6, 133.9, 129.7, 129.4, 129.2, 127.6, 125.4, 122.1, 120.3, 119.8, 116.3, 36.8, 29.7, 28.8, 22.6, 22.5. HRMS (ESI) m/z calculated for C₂₄H₂₂N₇O [M+H]⁺424.1880, found 424.1877.

### N-((1H-imidazol-2-yl)methyl)-3-fluoro-4-(8,9,10,11-tetrahydro-3H-pyrazolo[4,3-a]phenanthridin-7-yl)benzamide

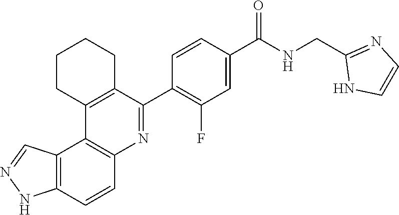 [SMILES: O=C(NCc1ncc[nH]1)c1ccc(-c2nc3ccc4[nH]ncc4c3c3c2CCCC3)c(F)c1]

Synthesized using Method D: Off white solid (109 mg, 1.0 mmol, 49.5%). ¹H NMR (500 MHZ, (CD₃)₂SO) δ 8.59 (s, 1H), 7.93-7.78 (m, 4H), 7.57 (t, J=7.6 Hz, 1H), 7.16 (s, 2H), 4.65 (d, J=5.5 Hz, 2H), 3.35 (t, J=6.5 Hz, 2H), 2.62 (t, J=6.2 Hz, 2H), 2.04-1.74 (m, 4H). 13C NMR (126 MHZ, (CD₃)₂SO) δ 165.6, 159.5 (d, 1J=252 Hz), 151.6, 151.1, 145.3, 144.0, 142.5, 136.5, 136.1, 131.9, 130.0, 129.5, 126.2, 125.4, 124.1, 122.5, 121.2, 120.4, 116.2, 115.3 (d, 2]=25 Hz), 36.9, 29.5, 27.4, 22.6, 22.2. HRMS (ESI) m/z calculated for C₂₅H₂₂FN₆O [M+H]⁺441.1833, found 441.1830.

### N-((1H-imidazol-2-yl)methyl)-4-(3,8,10,11-tetrahydropyrano[3,4-c]pyrazolo[4,3-f]quinolin-7-yl)benzamide

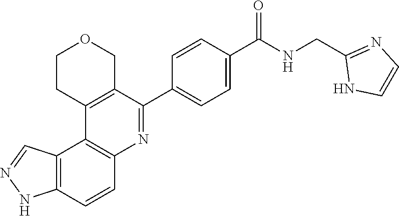 [SMILES: O=C(NCc1ncc[nH]1)c1ccc(-c2nc3ccc4[nH]ncc4c3c3c2COCC3)cc1]

Synthesized using Method D: Off white solid (117 mg, 0.5 mmol, 55.2%). ¹H NMR (500 MHz, (CD₃)₂SO) δ 8.59 (s, 1H), 8.04 (d, J=7.9 Hz, 2H), 7.94-7.83 (m, 2H), 7.67 (d, J=7.9 Hz, 2H), 6.95 (s, 2H), 4.79 (s, 2H), 4.55 (d, J=5.6 Hz, 2H), 4.16 (t, J=5.8 Hz, 2H), 3.41 (d, J=11.8 Hz, 2H). ¹³C NMR (126 MHZ, (CD₃)₂SO) δ 166.6, 156.2, 153.0, 147.0, 145.5, 142.6, 139.6, 139.5, 134.4, 129.5, 129.2, 129.2, 127.8, 127.5, 122.0, 121.6, 118.8, 116.2, 71.2, 66.9, 64.4, 37.6, 28.7, 25.7. HRMS (ESI) m/z calculated for C₂₄H₂₁N₆O₂ [M+H]⁺425.1720, found 425.1716.

### N-((1H-imidazol-2-yl)methyl)-4-(9,9-dioxido-3,8,10,11-tetrahydropyrazolo[4,3-f]thiopyrano[3,4-c]quinolin-7-yl)benzamide

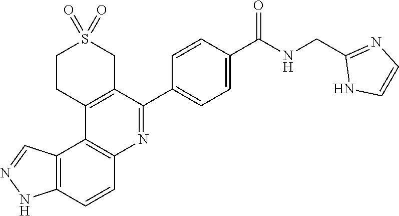 [SMILES: O=C(NCc1ncc[nH]1)c1ccc(-c2nc3ccc4[nH]ncc4c3c3c2CS(=O)(=O)CC3)cc1]

Synthesized using Method D: Off white solid (98 mg, 0.5 mmol, 41.5%). ¹H NMR (500 MHz, (CD₃)₂SO) δ 8.69 (s, 1H), 8.05 (dd, J=47.7, 8.4 Hz, 3H), 7.88 (d, J=9.1 Hz, 1H), 7.68 (d, J=7.8 Hz, 2H), 7.19 (s, 2H), 4.67 (d, J=5.4 Hz, 2H), 4.47 (s, 2H), 4.02 (t, J=6.7 Hz, 2H), 3.69 (t, J=6.8 Hz, 2H). ¹³C NMR (126 MHZ, (CD₃)₂SO) δ 166.8, 155.5, 145.5, 145.0, 142.5, 139.2, 136.0, 134.3, 129.6, 129.4, 128.1, 126.7, 125.4, 122.3, 121.3, 121.0, 120.1, 115.7, 52.0, 46.3, 36.8, 30.4. HRMS (ESI) m/z calculated for C₂₄H₂₁N₆O₃S [M+H]⁺473.1390, found 473.1386.

### N-((1H-tetrazol-5-yl)methyl)-4-(8,9,10,11-tetrahydro-3HI-pyrazolo[4,3-a]phenanthridin-7-yl)benzamide

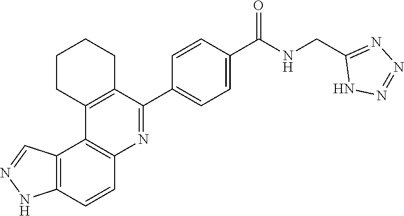 [SMILES: O=C(NCc1nnn[nH]1)c1ccc(-c2nc3ccc4[nH]ncc4c3c3c2CCCC3)cc1]

Synthesis of final compounds via “Click” reaction:

To a solution of the alkyne (1 eq) in DMF (2 mL) and water (0.5 mL) was added TMS-azide (2 eq). Catalytic amount of copper sulfide pentahydrate and sodium ascorbate was added. The reaction mixture was allowed to stir at 100° C. overnight. The reaction mixture was rotovapped, diluted in ethyl acetate and washed with water, and dried over sodium sulfate. It was then purified via column chromatography with 9% methanol, 1% ammonium hydroxide in water, and 90% ethyl acetate.

Off white solid. (36 mg, 1.0 mmol, 34.4%). ¹H NMR (500 MHZ, (CD₃)₂SO) δ 8.79 (d, J=3.9 Hz, 1H), 8.26-8.08 (m, 4H), 7.83 (dd, J=8.1, 3.8 Hz, 2H), 4.59 (dd, J=236.2, 5.5 Hz, 2H), 3.50 (t, J=6.2 Hz, 2H), 2.78 (t, J=6.2 Hz, 2H), 2.11-2.00 (m, 2H), 1.82 (ddt, J=11.9, 9.4, 4.1 Hz, 2H). ¹³C NMR (126 MHZ, (CD₃)₂SO) δ 166.6, 155.3, 151.5, 140.5, 137.4, 135.4, 131.3, 130.3, 130.1, 128.1, 128.0, 123.4, 122.5, 120.2, 117.9, 115.2, 33.9, 30.7, 28.3, 28.1, 21.9. HRMS (ESI) m/z calculated for C₂₃H₂₁N₈O [M+H]⁺425.1833, found 425.1831.

### N-((4-methyl-1H-imidazol-2-yl)methyl)-4-(8,9,10,11-tetrahydro-3H-pyrazolo[4,3-a]phenanthridin-7-yl)benzamide

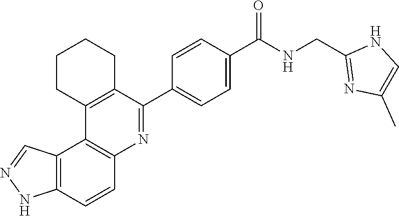 [SMILES: Cc1c[nH]c(CNC(=O)c2ccc(-c3nc4ccc5[nH]ncc5c4c4c3CCCC4)cc2)n1]

Method D: Off white solid. (79 mg, 0.5 mmol, 36.2%). ¹H NMR (500 MHZ, (CD₃)₂SO) δ 8.58 (s, 1H), 8.09-7.97 (m, 2H), 7.90-7.77 (m, 2H), 7.68-7.61 (m, 2H), 6.69-6.64 (m, 1H), 4.50 (d, J=5.6 Hz, 2H), 2.78 (t, J=6.2 Hz, 2H), 2.12 (d, J=1.1 Hz, 4H), 2.05 (d, J=16.2 Hz, 1H), 2.01 (td, J=9.0, 7.4, 4.7 Hz, 2H), 1.77 (tq, J=8.5, 6.0, 4.3 Hz, 2H), 1.24 (d, J=6.8 Hz, 1H). ¹³C NMR (126 MHZ, (CD₃)₂SO) δ 166.7, 156.2, 150.0, 144.6, 144.3, 143.9, 142.5, 133.9, 131.2, 129.6, 129.4, 129.2, 127.6, 122.1, 120.3, 118.0, 116.3, 37.5, 29.7, 28.8, 22.6, 22.5, 11.9.

### (S)-N-((tetrahydrofuran-2-yl)methyl)-4-(3,8,10,11-tetrahydropyrano[3,4-c]pyrazolo[4,3-f]quinolin-7-yl)benzamide

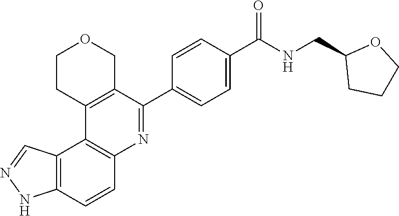 [SMILES: O=C(NC[C@@H]1CCCO1)c1ccc(-c2nc3ccc4[nH]ncc4c3c3c2COCC3)cc1]

Synthesized using Method D: Off white solid (98 mg, 0.5 mmol, 45.7%). ¹H NMR (500 MHz, (CD₃)₂SO) δ 8.62 (s, 1H), 8.55 (t, J=5.8 Hz, 1H), 8.00-7.86 (m, 4H), 7.69-7.65 (m, 2H), 4.79 (d, J=1.8 Hz, 2H), 4.16 (t, J=5.9 Hz, 2H), 3.79-3.69 (m, 2H), 3.63 (td, J=8.0, 6.7 Hz, 2H), 3.51 (dd, J=8.5, 5.4 Hz, 2H), 3.43 (d, J=11.9 Hz, 3H), 2.57-2.49 (m, 1H), 1.97 (dtd, J=12.2, 8.0, 5.6 Hz, 1H), 1.64 (ddt, J=12.5, 7.7, 6.1 Hz, 1H). ¹³C NMR (126 MHZ, (CD₃)₂SO) δ 166.6, 152.5, 143.7, 141.6, 140.6, 135.2, 131.3, 129.7, 129.6, 129.3, 128.7, 127.8, 127.7, 121.8, 116.0, 71.1, 67.3, 66.7, 64.4, 42.6, 31.0, 30.1, 28.8.

### (R)-N-((tetrahydrofuran-2-yl)methyl)-4-(3,8,10,11-tetrahydropyrano[3,4-c]pyrazolo[4,3-f]quinolin-7-yl)benzamide

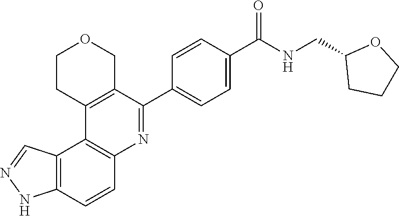 [SMILES: O=C(NC[C@H]1CCCO1)c1ccc(-c2nc3ccc4[nH]ncc4c3c3c2COCC3)cc1]

Synthesized using Method D: Off white solid (121 mg, 0.5 mmol, 56.5%). ¹H NMR (500 MHz, (CD₃)₂SO) δ 8.60 (s, 1H), 8.05 (d, J=7.9 Hz, 2H), 7.97-7.90 (m, 2H), 7.86 (d, J=9.2 Hz, 1H), 7.69 (dd, J=17.1, 7.9 Hz, 2H), 4.79 (s, 2H), 4.15 (t, J=5.9 Hz, 2H), 3.41 (d, J=6.0 Hz, 3H). ¹³C NMR (126 MHZ, (CD₃)₂SO) δ 167.5, 152.8, 144.5, 144.0, 139.6, 131.2, 129.7, 129.5, 129.4, 129.2, 127.6, 127.5, 121.6, 116.1, 71.1, 67.3, 66.8, 64.4, 42.6, 30.11, 28.7.

### N-(tetrahydro-2H-pyran-4-yl)-4-(3,8,10,11-tetrahydropyrano[3,4-c]pyrazolo[4,3-f]quinolin-7-yl)benzamide

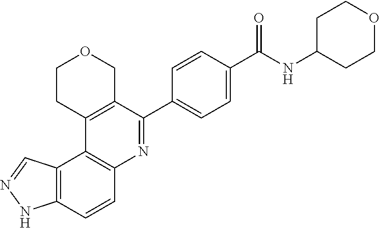 [SMILES: O=C(NC1CCOCC1)c1ccc(-c2nc3ccc4[nH]ncc4c3c3c2COCC3)cc1]

Synthesized using Method D: Off white solid (96 mg, 0.5 mmol, 44.8%) ¹H NMR (500 MHZ, (CD₃)₂SO) δ 8.27 (d, J=7.6 Hz, 1H), 7.94 (dd, J=31.0, 8.4 Hz, 4H), 7.65 (d, J=8.2 Hz, 2H), 4.78 (s, 2H), 4.16 (t, J=5.9 Hz, 2H), 4.04 (tdd, J=13.4, 9.3, 5.1 Hz, 1H), 3.97-3.83 (m, 2H), 3.41 (td, J=11.5, 2.3 Hz, 4H), 1.81 (ddd, J=12.4, 4.7, 2.1 Hz, 2H), 1.63 (qd, J=11.8, 4.4 Hz, 2H). ¹³C NMR (126 MHZ, (CD₃)₂SO) δ 165.9, 156.9, 153.1, 148.7, 144.4, 142.4, 139.5, 139.1, 136.0, 135.1, 129.6, 129.1, 127.7, 121.5, 116.3, 115.2, 66.9, 66.7, 64.4, 46.4, 32.9, 28.7.

### N-((3-methyloxetan-3-yl)methyl)-4-(3,8,10,11-tetrahydropyrano[3,4-c]pyrazolo[4,3-f]quinolin-7-yl)benzamide

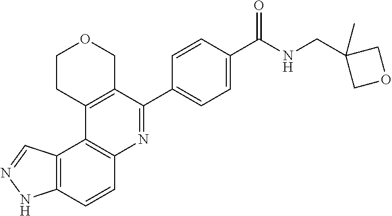 [SMILES: CC1(CNC(=O)c2ccc(-c3nc4ccc5[nH]ncc5c4c4c3COCC4)cc2)COC1]

Synthesized using Method D: Off white solid (87 mg, 0.5 mmol, 40.6%) ¹H NMR (500 MHz, (CD₃)₂SO) δ 8.58 (t, J=6.1 Hz, 1H), 8.06-7.79 (m, 2H), 7.77-7.58 (m, 1H), 4.80 (s, 1H), 4.52 (d, J=5.7 Hz, 1H), 4.28-4.08 (m, 2H), 3.47 (dd, J=50.8, 5.9 Hz, 2H), 3.18 (s, 3H), 1.29 (s, 1H). ¹³C NMR (126 MHZ, (CD₃)₂SO) δ 167.3, 158.7, 153.0, 144.4, 142.5, 139.5, 139.4, 134.9, 129.6, 129.2, 129.0, 127.7, 121.5, 116.3, 80.0, 79.8, 66.9, 64.4, 46.2, 28.7, 22.5.

### N-(2-hydroxy-2-methylpropyl)-4-(3,8,10,11-tetrahydropyrano[3,4-c]pyrazolo[4,3-f]quinolin-7-yl)benzamide

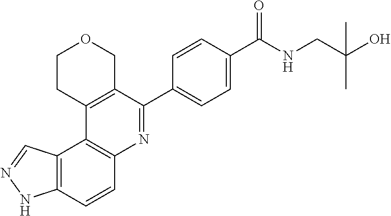 [SMILES: CC(C)(O)CNC(=O)c1ccc(-c2nc3ccc4[nH]ncc4c3c3c2COCC3)cc1]

Synthesized using Method D: Off white solid (72 mg, 0.5 mmol, 34.6%). ¹H NMR (500 MHz, (CD₃)₂SO) δ 8.19 (t, J=6.1 Hz, 1H), 8.06-7.78 (m, 4H), 7.74-7.56 (m, 2H), 4.80 (s, 2H), 4.16 (t, J=5.9 Hz, 2H), 3.36 (dd, J=43.4, 6.0 Hz, 4H), 1.15 (d, J=1.8 Hz, 6H). ¹³C NMR (126 MHz, (CD₃)₂SO) δ 168.3, 154.0, 144.4, 141.6, 141.0, 139.7, 134.9, 131.7, 128.7, 127.5, 121.4, 126.3, 121.3, 120.4, 117.0, 113.6, 71.5, 67.8, 64.4, 50.4, 28.7, 26.5, 26.3.

### General Scheme for the Synthesis of Hydroxamic Acid Analogs:

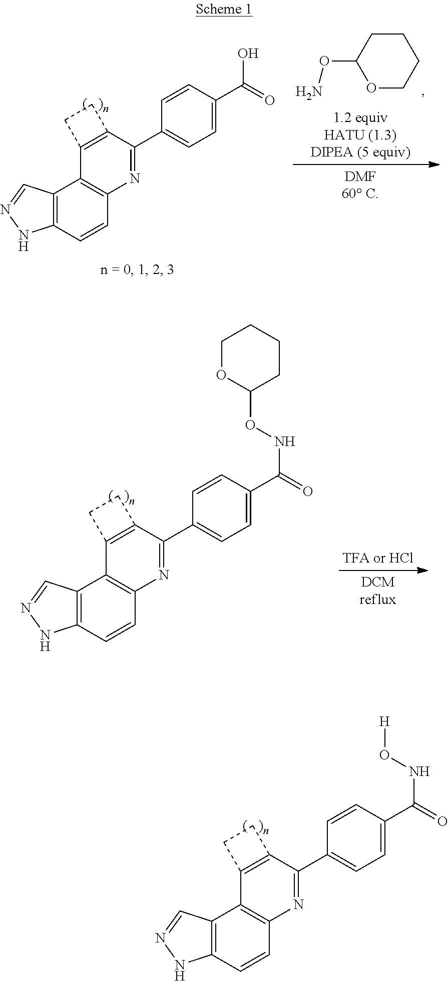

### General Procedure for the Amide Coupling:

In a 20 mL capped vial, acid intermediate (0.5 mmol), O-(tetrahydro-2H-pyran-2-yl) hydroxylamine (0.7 mmol) and hexafluorophosphate azabenzotriazole tetramethyl uronium (HATU) (1.3 eq) were dissolved in anhydrous DMF (3 mL). Followed by addition of DIPEA (5 eq). Reaction was stirred at 80° C. for an overnight. After completion, the reaction was concentrated and purified via silica gel column chromatography using ethyl acetate and hexanes (30:70 to 70:30).

### General Procedure for the Deprotection of Tetrahydropyranyl Ethers:

In a 5 mL vial tetrahydropyranyl protected intermediate (0.25 mmol) was dissolved in dichloromethane (1.5 mL). Followed by addition of trifluoroacetic acid or hydrochloric acid (2 equiv). Reaction was refluxed in capped vial for 2 hours to get the desired deprotected product.

### N-hydroxy-4-(8,9,10,11-tetrahydro-3H-pyrazolo[4,3-a]phenanthridin-7-yl)benzamide (Compound HSD1993)

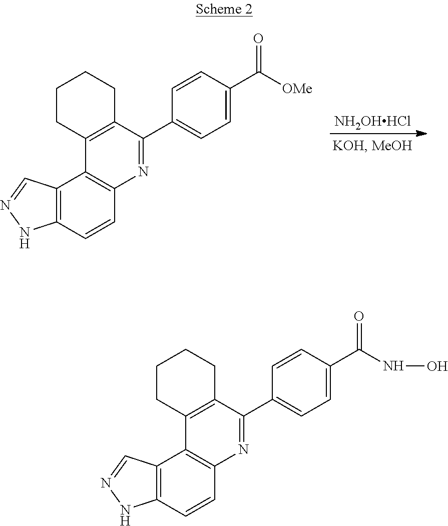 [SMILES: COC(=O)c1ccc(-c2nc3ccc4[nH]ncc4c3c3c2CCCC3)cc1 ; "NH2OH·HCl" ; O=C(NO)c1ccc(-c2nc3ccc4[nH]ncc4c3c3c2CCCC3)cc1]

Pursuant to Scheme 2, solution A was prepared by suspending substrate ester (0.25 mmol) in methanol (2 mL) and a separate solution B was prepared by dissolving crushed potassium hydroxide (KOH) (10 equiv) and hydroxylamine hydrochloride (5 equiv) in methanol (4 mL), and stirred for 30 minutes at ambient temperature. Solution B filtrate was added to solution A and continued to stir for overnight. After completion, solvent was removed under reduced pressure and the resulted solid was dissolved in water. The pH was adjusted to 7 with aqueous solution of HCl (IN) and the resulting precipitate was filtered and dried under high vacuum which was purified by washing and recrystallization with ethanol to afford the desired product as off-white solid (85 mg, 24%).

¹H NMR (500 MHZ, DMSO-d₆) δ 11.31 (s, 1H), 9.09 (s, 1H), 8.57 (s, 1H), 7.90-7.83 (m, 3H), 7.81-7.80 (m, 1H), 7.63 (d, J=8.3 Hz, 2H), 3.34-3.27 (m, 2H), 2.76 (t, J=6.1 Hz, 2H), 2.02-1.94 (m, 2H), 1.78-1.67 (m, 2H); ¹³C NMR (126 MHZ, DMSO-d₆) δ 164.5, 162.7, 156.0, 143.9, 143.7, 142.5, 138.7, 136.3, 132.4, 129.5, 129.2, 127.0, 122.0, 116.3, 114.7, 29.6, 28.8, 22.5, 22.4.

### 4-(1-Methyl-8,9,10,11-tetrahydro-3HI-pyrazolo[4,3-a]phenanthridin-7-yl)-N-((tetrahydro-2H-pyran-2-yl)oxy)benzamide (Compound HSD19931P)

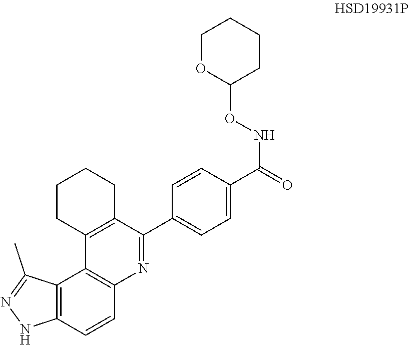 [SMILES: Cc1n[nH]c2ccc3nc(-c4ccc(C(=O)NOC5CCCCO5)cc4)c4c(c3c12)CCCC4]

Off-white solid (41%); ¹H NMR (500 MHz, DMSO-d₆) δ 11.72 (s, 1H), 7.87 (d, J=8.0 Hz, 2H), 7.77-7.67 (m, 2H), 7.62 (d, J=7.9 Hz, 2H), 5.03 (d, J=3.0 Hz, 1H), 4.07 (t, J=9.7 Hz, 1H), 3.57-3.52 (m, 1H), 3.46-3.35 (m, 3H), 2.83 (s, 3H), 2.75 (s, 2H), 1.82-1.69 (m, 7H), 1.63-1.50 (m, 3H); ¹³C NMR (126 MHz, DMSO-d₆) δ 164.5, 155.6, 144.3, 143.9, 143.0, 142.5, 141.2, 131.9, 129.9, 129.5, 128.4, 127.4, 123.8, 114.8, 114.6, 101.4, 61.8, 31.7, 28.3, 28.0, 25.2, 22.5, 22.1, 20.4, 18.7.

### N-hydroxy-4-(1-methyl-8,9,10,11-tetrahydro-3HI-pyrazolo[4,3-a]phenanthridin-7-yl)benzamide (Compound HSD19931)

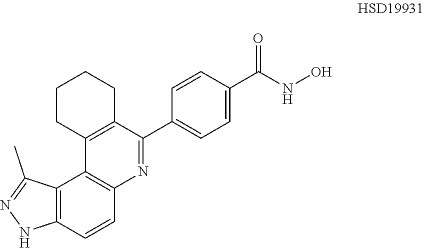 [SMILES: Cc1n[nH]c2ccc3nc(-c4ccc(C(=O)NO)cc4)c4c(c3c12)CCCC4]

Off-white solid (58%); ¹H NMR (500 MHz, DMSO-d₆) § 11.40 (s, 1H), 8.00 (d, J=9.1 Hz, 1H), 7.94 (d, J=7.9 Hz, 2H), 7.83 (d, J=9.1 Hz, 1H), 7.73 (d, J=7.9 Hz, 2H), 3.55 (t, J=5.5 Hz, 2H), 2.91 (s, 3H), 2.76 (t, J=5.8 Hz, 2H), 2.48 (t, J=2.0 Hz, 2H), 1.89-1.73 (m, 4H).

### 4-(9,9-Dioxido-3,8,10,11-tetrahydropyrazolo[4,3-f]thiopyrano[3,4-c]quinolin-7-yl)-N-((tetrahydro-2/-pyran-2-yl)oxy)benzamide

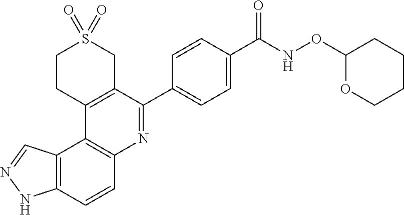 [SMILES: O=C(NOC1CCCCO1)c1ccc(-c2nc3ccc4[nH]ncc4c3c3c2CS(=O)(=O)CC3)cc1]

Off-white solid (36%); ¹H NMR (500 MHZ, DMSO-d₆) δ 11.77 (s, 1H), 8.68 (s, 1H), 8.00 (dd, J=9.1, 0.9 Hz, 1H), 7.96-7.89 (m, 3H), 7.70-7.62 (m, 2H), 5.08-4.98 (m, 1H), 4.54-4.44 (m, 2H), 4.12-3.96 (m, 3H), 3.69 (t, J=6.6 Hz, 2H), 3.59-3.51 (m, 1H), 1.80-1.67 (m, 3H), 1.63-1.48 (m, 3H); ¹³C NMR (126 MHz, DMSO) δ 164.3, 155.4, 144.6, 142.5, 139.1, 136.6, 132.7, 129.7, 129.5, 127.8, 122.4, 121.1, 116.3, 115.9, 101.5, 61.8, 51.7, 45.9, 30.6, 28.3, 25.2, 18.7.

### 4-(9,9-Dioxido-3,8,10,11-tetrahydropyrazolo[4,3-f]thiopyrano[3,4-c]quinolin-7-yl)-N-hydroxybenzamide (Compound 19932)

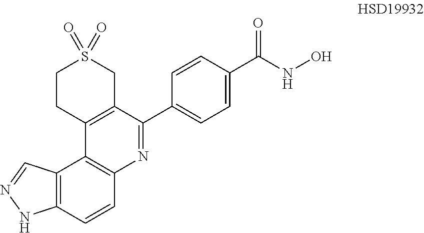 [SMILES: O=C(NO)c1ccc(-c2nc3ccc4[nH]ncc4c3c3c2CS(=O)(=O)CC3)cc1]

Off-white solid (61%); ¹H NMR (500 MHZ, DMSO-d₆) δ 11.34 (s, 1H), 9.12 (s, 1H), 8.70 (d, J=12.2 Hz, 1H), 8.00 (d, J=9.0 Hz, 1H), 7.91 (d, J=8.0 Hz, 2H), 7.64 (d, J=8.0 Hz, 2H), 4.50 (s, 2H), 4.01 (s, 2H), 3.69 (t, J=6.7 Hz, 2H).

### 2-Fluoro-N-((tetrahydro-2H-pyran-2-yl)oxy)-5-(8,9,10,11-tetrahydro-3H-pyrazolo[4,3-a]phenanthridin-7-yl)benzamide

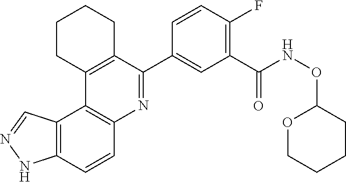 [SMILES: O=C(NOC1CCCCO1)c1cc(-c2nc3ccc4[nH]ncc4c3c3c2CCCC3)ccc1F]

Off-white solid (40%); ¹H NMR (500 MHZ, DMSO-d₆) § 11.49 (s, 1H), 8.59 (s, 1H), 7.92-7.81 (m, 1H), 7.78-7.72 (m, 1H), 7.69 (dd, J=6.7, 2.3 Hz, 1H), 7.40 (t, J=9.9, 8.5 Hz, 1H), 5.03 (s, 1H), 4.06-3.90 (m, 1H), 3.51 (d, J=11.3 Hz, 1H), 3.37-3.32 (m, 2H), 2.78 (t, J=6.1 Hz, 2H), 2.01 (t, J=6.0 Hz, 2H), 1.84-1.65 (m, 5H), 1.53 (s, 3H).

### 2-Fluoro-N-hydroxy-5-(8,9,10,11-tetrahydro-3H-pyrazolo[4,3-a]phenanthridin-7-yl)benzamide (Compound HSD19933)

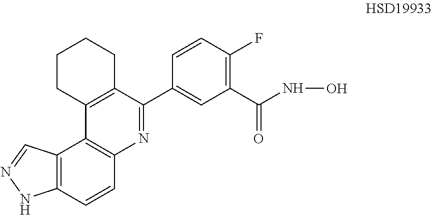 [SMILES: O=C(NO)c1cc(-c2nc3ccc4[nH]ncc4c3c3c2CCCC3)ccc1F]

Off-white solid (52%); ¹H NMR (500 MHZ, DMSO-d₆) δ 11.12 (s, 1H), 8.73 (s, 1H), 8.06 (d, J=9.2 Hz, 1H), 7.92 (d, J=9.1 Hz, 1H), 7.85-7.78 (m, 2H), 7.49 (t, J=9.4 Hz, 1H), 3.42 (t, J=6.4 Hz, 2H), 2.78 (t, J=6.1 Hz, 2H), 2.12-1.99 (m, 2H), 1.87-1.73 (m, 2H).

### 4-(9-Methyl-3H-pyrazolo[4,3-f]quinolin-7-yl)-N-((tetrahydro-2H-pyran-2-yl)oxy)benzamide

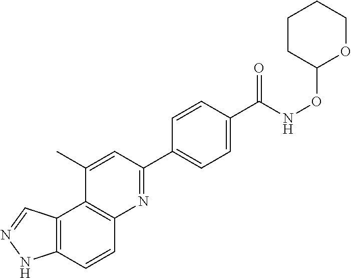 [SMILES: Cc1cc(-c2ccc(C(=O)NOC3CCCCO3)cc2)nc2ccc3[nH]ncc3c12]

Off-white solid (39%); ¹H NMR (500 MHZ, DMSO-d₆) δ 11.74 (s, 1H), 8.58 (s, 1H), 8.37 (d, J=8.1 Hz, 2H), 8.21 (s, 1H), 8.00-7.88 (m, 4H), 5.11-4.92 (m, 1H), 4.09 (d, J=11.5 Hz, 1H), 3.54 (dd, J=9.7, 5.4 Hz, 1H), 2.97 (s, 3H), 1.73 (s, 3H), 1.60-1.49 (m, 3H); ¹³C NMR (126 MHz, DMSO) δ 164.4, 152.2, 146.1, 144.6, 142.0, 138.5, 136.1, 132.9, 129.8, 128.2, 127.1, 122.2, 120.9, 116.9, 115.7, 101.5, 61.8, 28.4, 25.2, 22.8, 18.8.

### N-hydroxy-4-(9-methyl-3//-pyrazolo[4,3-f]quinolin-7-yl)benzamide (Compound 19934)

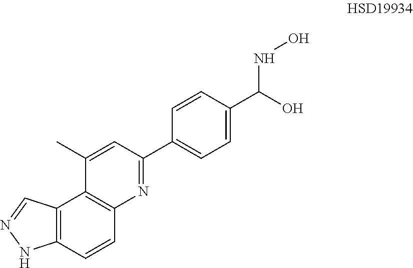 [SMILES: Cc1cc(-c2ccc(C(O)NO)cc2)nc2ccc3[nH]ncc3c12]

Off-white solid (60%); ¹H NMR (500 MHZ, DMSO-d₆) δ 11.36 (s, 1H), 8.64 (s, 1H), 8.32 (d, J=8.4 Hz, 2H), 8.23 (s, 1H), 8.04-7.96 (m, 2H), 7.94 (d, J=8.4 Hz, 2H), 2.98 (s, 3H).

### 4-(9-Cyclobutyl-3HI-pyrazolo[4,3-f]quinolin-7-yl)-N-((tetrahydro-2H-pyran-2-yl)oxy)benzamide (Compound HSD19935P)

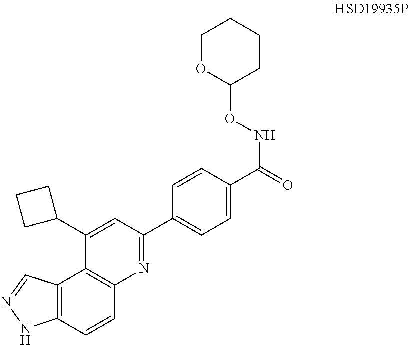 [SMILES: O=C(NOC1CCCCO1)c1ccc(-c2cc(C3CCC3)c3c(ccc4[nH]ncc43)n2)cc1]

Off-white solid (53%); ¹H NMR (500 MHZ, Methanol-d₄) δ 8.57 (s, 1H), 8.25 (d, J=8.1 Hz, 2H), 8.07 (s, 1H), 8.02 (d, J=8.5 Hz, 1H), 7.96 (d, J=8.1 Hz, 2H), 7.89 (d, J=9.2 Hz, 1H), 5.11 (d, J=2.9 Hz, 1H), 4.43 (s, 1H), 4.17 (t, J=10.7 Hz, 1H), 3.72-3.53 (m, 1H), 2.80 (s, 2H), 2.76-2.67 (m, 2H), 2.40-2.31 (m, 2H), 2.29-2.20 (m, 1H), 2.11-2.01 (m, 1H), 1.96-1.78 (m, 2H), 1.74-1.56 (m, 2H).

### 4-(9-Cyclobutyl-3HI-pyrazolo[4,3-f]quinolin-7-yl)-N-hydroxybenzamide (Compound HSD19935)

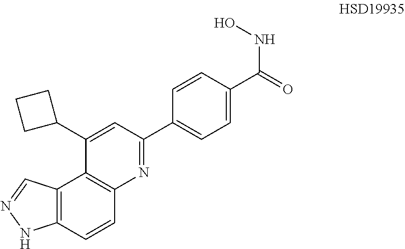 [SMILES: O=C(NO)c1ccc(-c2cc(C3CCC3)c3c(ccc4[nH]ncc43)n2)cc1]

Off-white solid (55%); ¹H NMR (500 MHZ, DMSO-d₆) § 11.35 (s, 1H), 8.65 (s, 1H), 8.39 (d, J=8.4 Hz, 2H), 8.19 (s, 1H), 7.97-7.90 (m, 4H), 4.44 (p, J=8.1 Hz, 1H), 2.71-2.57 (m, 2H), 2.41-2.29 (m, 2H), 2.27-2.08 (m, 1H), 2.03-1.85 (m, 1H).

### N-hydroxy-4-(1-methyl-9-morpholino-8,9,10,11-tetrahydro-3H-pyrazolo[4,3-a]phenanthridin-7-yl)benzamide (Compound HSD19936)

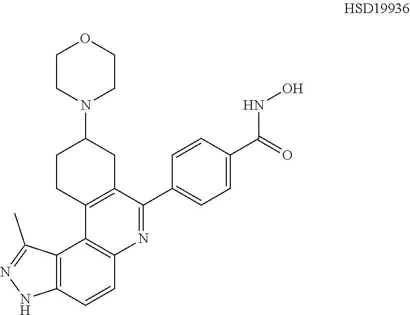 [SMILES: Cc1n[nH]c2ccc3nc(-c4ccc(C(=O)NO)cc4)c4c(c3c12)CCC(N1CCOCC1)C4]

Yellow solid (68%); ¹H NMR (500 MHZ, DMSO-d₆) δ 11.44 (s, 1H), 11.04 (s, 1H), 7.95 (d, J=8.1 Hz, 3H), 7.84 (d, J=9.0 Hz, 1H), 7.73 (d, J=7.9 Hz, 2H), 4.01-3.94 (m, 2H), 3.82-3.72 (m, 5H), 3.55-3.51 (m, 2H), 3.32-3.17 (m, 4H), 3.08-3.00 (m, 1H), 2.90 (s, 3H), 1.88-1.82 (m, 1H).

### 4-(5-Fluoro-8,9,10,11-tetrahydro-3HI-pyrazolo[4,3-a]phenanthridin-7-yl)-N-hydroxybenzamide (Compound HSD19937)

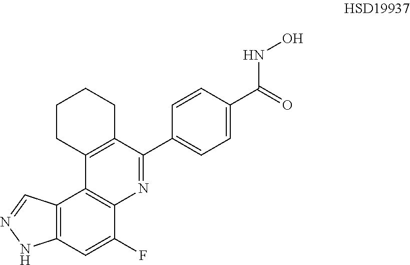 [SMILES: O=C(NO)c1ccc(-c2nc3c(F)cc4[nH]ncc4c3c3c2CCCC3)cc1]

Off-white solid (59%); ¹H NMR (500 MHZ, DMSO-d₆) δ 11.33 (s, 1H), 8.61 (s, 1H), 7.87 (d, J=8.3 Hz, 2H), 7.71 (d, J=10.0 Hz, 1H), 7.67-7.60 (m, 2H), 3.30 (t, J=6.3 Hz, 2H), 2.78 (t, J=6.1 Hz, 2H), 2.03-1.94 (m, 2H), 1.82-1.68 (m, 2H).

### N-methoxy-4-(8,9,10,11-tetrahydro-3H-pyrazolo[4,3-a]phenanthridin-7-yl)benzamide (Compound HSD199310)

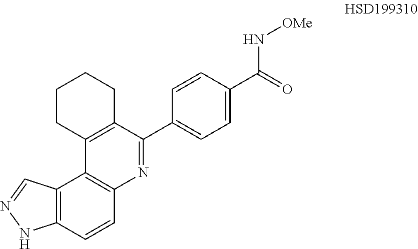 [SMILES: CONC(=O)c1ccc(-c2nc3ccc4[nH]ncc4c3c3c2CCCC3)cc1]

Off-white solid (65%); ¹H NMR (500 MHZ, DMSO-d₆) δ 11.83 (s, 1H), 8.57 (s, 1H), 7.97-7.78 (m, 4H), 7.66 (d, J=8.2 Hz, 2H), 3.74 (s, 3H), 3.36-3.32 (m, 2H), 2.76 (t, J=6.1 Hz, 2H), 2.08-1.90 (m, 2H), 1.82-1.47 (m, 2H); ¹³C NMR (126 MHz, DMSO) & 164.3, 156.0, 144.4, 143.6, 142.6, 138.7, 136.4, 131.9, 129.6, 129.3, 127.2, 122.0, 116.4, 114.5, 63.7, 29.6, 28.8, 22.5, 22.4.

### 5-(1-Methyl-8,9,10,11-tetrahydro-3H-pyrazolo[4,3-a]phenanthridin-7-yl)-N-((tetrahydro-2//-pyran-2-yl)oxy) picolinamide

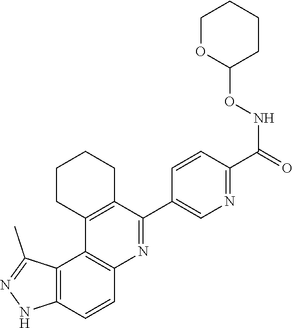 [SMILES: Cc1n[nH]c2ccc3nc(-c4ccc(C(=O)NOC5CCCCO5)nc4)c4c(c3c12)CCCC4]

Off-white solid (57%); ¹H NMR (500 MHZ, DMSO-d₆) δ 11.90 (s, 1H), 8.79 (d, J=2.2 Hz, 1H), 8.19 (dd, J=8.0, 2.1 Hz, 1H), 8.09 (d, J=8.0 Hz, 1H), 7.76 (s, 1H), 5.11 (d, J=2.7 Hz, 1H), 4.24-4.11 (m, 1H), 3.54-3.36 (m, 4H), 2.95-2.75 (m, 6H), 1.84-1.66 (m, 7H), 1.62-1.52 (m, 3H); ¹³C NMR (126 MHz, DMSO) δ 161.5, 152.8, 149.1, 149.0, 144.1, 143.1, 142.8, 141.3, 139.4, 138.5, 129.9, 128.8, 122.0, 115.1, 114.5, 100.9, 61.4, 31.7, 28.2, 27.9, 25.2, 22.4, 22.1, 20.4, 18.4.

### N-hydroxy-5-(1-methyl-8,9,10,11-tetrahydro-3/I-pyrazolo[4,3-a]phenanthridin-7-yl) picolinamide (Compound HSD199312)

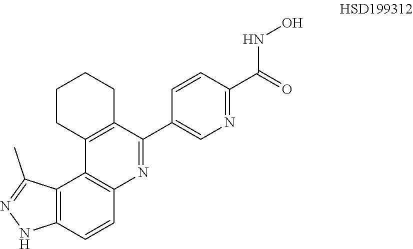 [SMILES: Cc1n[nH]c2ccc3nc(-c4ccc(C(=O)NO)nc4)c4c(c3c12)CCCC4]

Off-white solid (70%); ¹H NMR (500 MHZ, DMSO-d₆) δ 11.58 (s, 1H), 8.84 (s, 1H), 8.26 (dd, J=8.1, 2.2 Hz, 1H), 8.13 (d, J=8.0 Hz, 1H), 7.94 (d, J=9.2 Hz, 1H), 7.80 (d, J=9.1 Hz, 1H), 3.64-3.41 (m, 2H), 2.90 (s, 3H), 2.84-2.74 (m, 2H), 1.99-1.72 (m, 4H).

### 6-(1-Methyl-8,9,10,11-tetrahydro-3H-pyrazolo[4,3-a]phenanthridin-7-yl)-N-((tetrahydro-2H-pyran-2-yl)oxy) nicotinamide

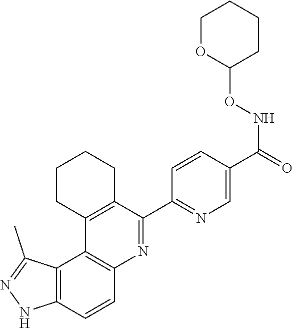 [SMILES: Cc1n[nH]c2ccc3nc(-c4ccc(C(=O)NOC5CCCCO5)cn4)c4c(c3c12)CCCC4]

Off-white solid (50%); ¹H NMR (500 MHZ, DMSO-d₆) δ 11.91 (s, 1H), 9.07-8.84 (m, 1H), 8.27 (dd, J=8.2, 2.3 Hz, 1H), 7.91 (d, J=8.2 Hz, 1H), 7.80-7.55 (m, 1H), 5.05 (s, 1H), 4.14-3.98 (m, 1H), 3.56 (dd, J=10.2, 5.2 Hz, 1H), 3.42 (s, 2H), 3.05-2.94 (m, 2H), 2.86 (s, 3H), 1.86-1.66 (m, 7H), 1.64-1.45 (m, 3H).

### N-hydroxy-6-(1-methyl-8,9,10,11-tetrahydro-3/I-pyrazolo[4,3-a]phenanthridin-7-yl) nicotinamide (HSD199313)

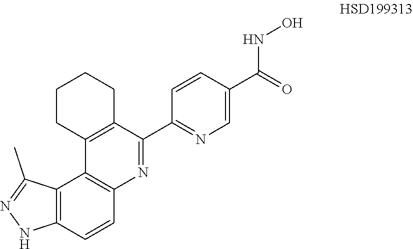 [SMILES: Cc1n[nH]c2ccc3nc(-c4ccc(C(=O)NO)cn4)c4c(c3c12)CCCC4]

Off-white solid (61%); ¹H NMR (500 MHZ, DMSO-d₆) § 11.52 (s, 1H), 9.03 (d, J=2.2 Hz, 1H), 8.31 (dd, J=8.1, 2.3 Hz, 1H), 7.96 (d, J=8.1 Hz, 1H), 7.89 (d, J=9.1 Hz, 1H), 7.79 (d, J=9.1 Hz, 1H), 3.66-3.24 (m, 2H), 2.99-2.94 (m, 2H), 2.89 (s, 3H), 1.97-1.48 (m, 4H).

### N-hydroxy-4-(3,8,10,11-tetrahydropyrazolo[4,3-f]thiopyrano[3,4-c]quinolin-7-yl)benzamide (Compound HSD199315)

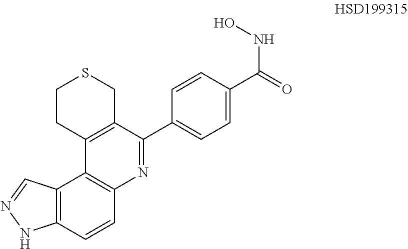 [SMILES: O=C(NO)c1ccc(-c2nc3ccc4[nH]ncc4c3c3c2CSCC3)cc1]

Off-white solid (56%); ¹H NMR (500 MHz, DMSO-d₆) δ 11.38 (s, 1H), 8.73 (s, 1H), 8.03 (d, J=9.1 Hz, 1H), 7.96-7.91 (m, 2H), 7.89 (d, J=9.1 Hz, 1H), 7.76-7.70 (m, 2H), 3.88 (s, 2H), 3.68-3.62 (m, 1H), 3.17 (t, J=6.1 Hz, 2H).

### 4-(5-Methyl-8,9,10,11-tetrahydro-3H-pyrazolo[4,3-a]phenanthridin-7-yl)-N-((tetrahydro-2H-pyran-2-yl)oxy)benzamide

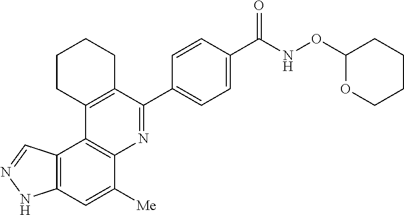 [SMILES: Cc1cc2[nH]ncc2c2c3c(c(-c4ccc(C(=O)NOC5CCCCO5)cc4)nc12)CCCC3]

Off-white solid (38%); ¹H NMR (500 MHZ, DMSO-d₆) δ 8.52 (s, 1H), 7.90-7.81 (m, 2H), 7.77-7.73 (m, 1H), 7.72-7.69 (m, 2H), 5.09-4.97 (m, 1H), 4.15-4.00 (m, 1H), 3.60-3.49 (m, 1H), 2.81 (t, J=6.1 Hz, 2H), 2.69 (s, 3H), 2.08-1.96 (m, 2H), 1.79-1.60 (m, 5H), 1.60-1.49 (m, 3H).

### N-hydroxy-4-(5-methyl-8,9,10,11-tetrahydro-3/I-pyrazolo[4,3-a]phenanthridin-7-yl)benzamide (Compound HSD199316)

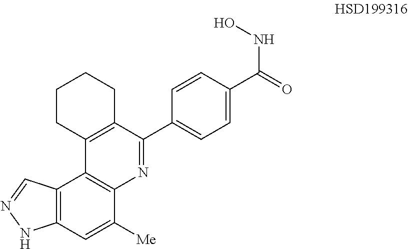 [SMILES: Cc1cc2[nH]ncc2c2c3c(c(-c4ccc(C(=O)NO)cc4)nc12)CCCC3]

Off-white solid (62%); ¹H NMR (500 MHZ, DMSO-d₆) δ 11.30 (s, 1H), 8.53 (s, 1H), 7.90-7.83 (m, 2H), 7.75 (s, 1H), 7.73-7.66 (m, 2H), 3.32 (t, J=6.6 Hz, 2H), 2.81 (t, J=6.1 Hz, 2H), 2.08-1.96 (m, 3H), 1.81-1.68 (m, 3H).

### 4-(5-Methoxy-8,9,10,11-tetrahydro-3H-pyrazolo[4,3-a]phenanthridin-7-yl)-N-((tetrahydro-2H-pyran-2-yl)oxy)benzamide

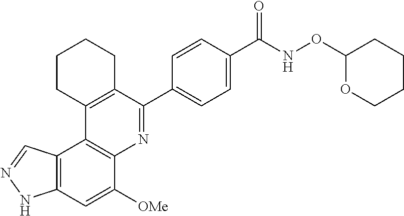 [SMILES: COc1cc2[nH]ncc2c2c3c(c(-c4ccc(C(=O)NOC5CCCCO5)cc4)nc12)CCCC3]

Off-white solid (51%); ¹H NMR (500 MHz, DMSO-d₆) δ 11.73 (s, 1H), 8.46 (d, J=7.2 Hz, 1H), 7.88 (d, J=8.3 Hz, 2H), 7.65-7.59 (m, 2H), 7.17 (s, 1H), 5.04 (d, J=3.0 Hz, 1H), 4.12-4.04 (m, 1H), 3.96 (s, 3H), 3.55 (dd, J=9.4, 5.9 Hz, 1H), 3.38-3.29 (m, 2H), 2.74 (t, J=6.0 Hz, 2H), 2.03-1.94 (m, 2H), 1.77-1.71 (m, 5H), 1.60-1.52 (m, 3H).

### N-hydroxy-4-(5-methoxy-8,9,10,11-tetrahydro-3H-pyrazolo[4,3-a]phenanthridin-7-yl)benzamide (Compound HSD199317)

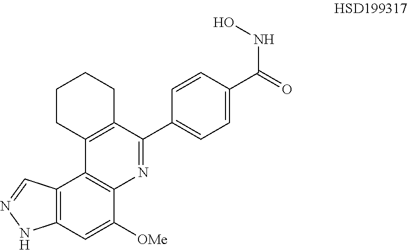 [SMILES: COc1cc2[nH]ncc2c2c3c(c(-c4ccc(C(=O)NO)cc4)nc12)CCCC3]

Off-white solid (70%); ¹H NMR (500 MHZ, DMSO-d₆) § 11.35 (s, 1H), 8.52 (s, 1H), 8.00-7.80 (m, 2H), 7.73-7.56 (m, 2H), 7.29 (s, 1H), 3.99 (d, J=1.4 Hz, 3H), 3.32 (t, J=6.4 Hz, 2H), 2.72 (t, J=6.1 Hz, 2H), 2.19-1.94 (m, 2H), 1.75 (q, J=5.5 Hz, 2H).

### Example 2

### Evaluation of CDK Inhibitor Compounds in Cancer Cells

Molm-14 (a human acute myeloid leukemia (AML) cell line), Molm-14 D835Y (a human AML cell line with a mutation at D835Y), Molm-14 F691L (a human AML cell line with a mutation at F691L which is a common cause of acquired resistance to FLT3 inhibitors), SNU-16 (a human gastric carcinoma cell line), and KCL22-IR (a chronic myeloid leukemia cell line) were cultured in RPMI-1640 medium with L-glutamine and sodium bicarbonate supplemented with 10% fetal bovine serum (FBS) and 1% Penicillin/streptomycin. Cells were seeded at 2.1E4 cells per mL in 96 well plates with 190 μL in each well. The plates were incubated in a 5% CO₂ incubator for 24 hours. Cells were then treated with the compounds hereof at desired concentrations or a dimethylsulfoxide (DMSO) control and incubated at in a 5% CO₂ incubator for 72 hours.

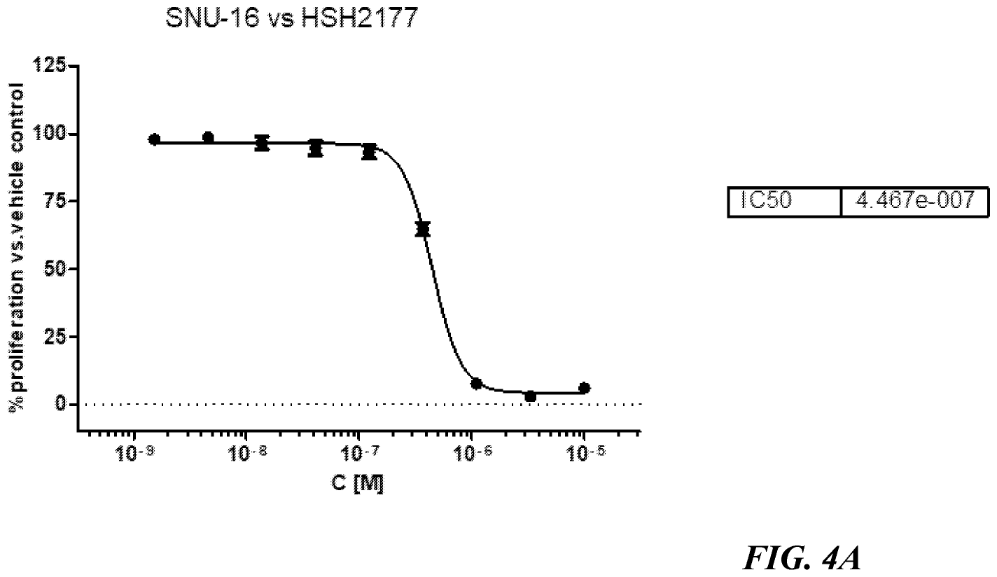

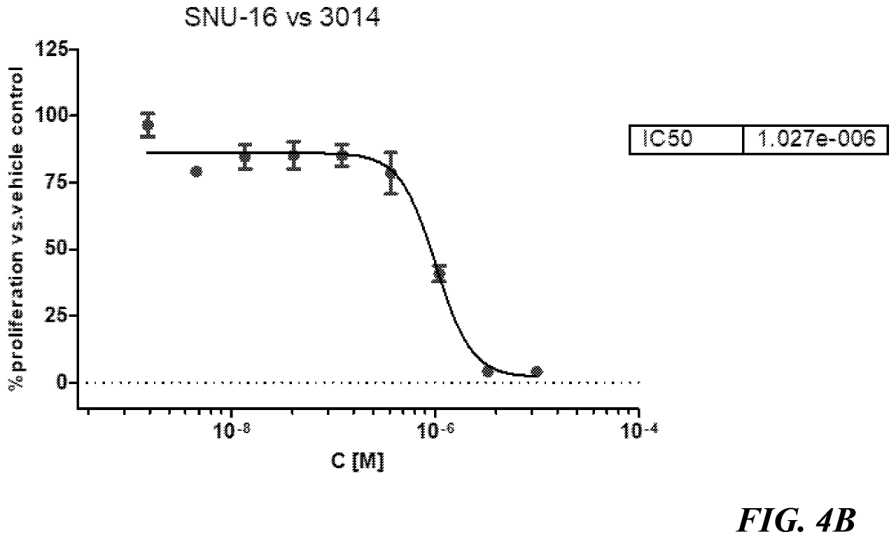

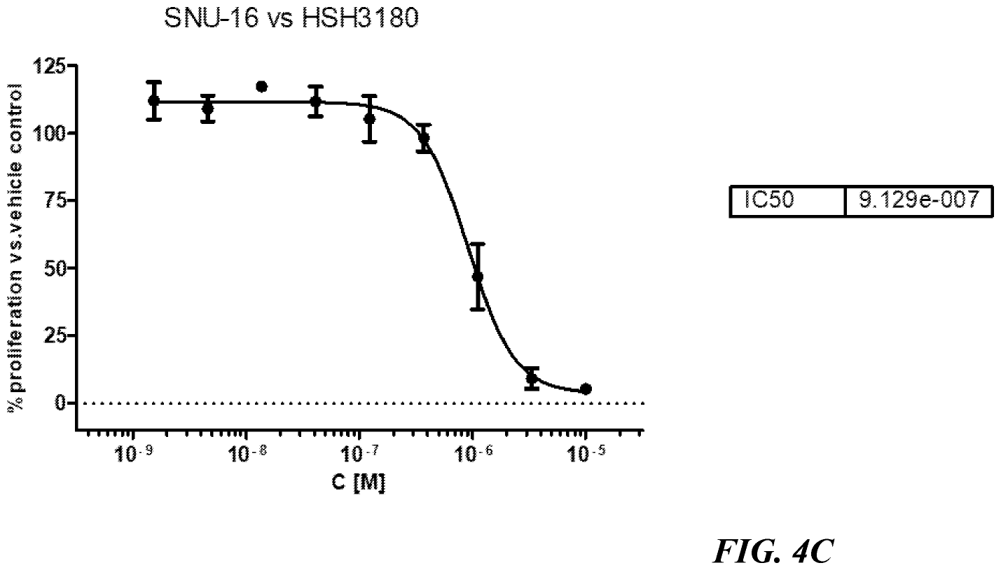

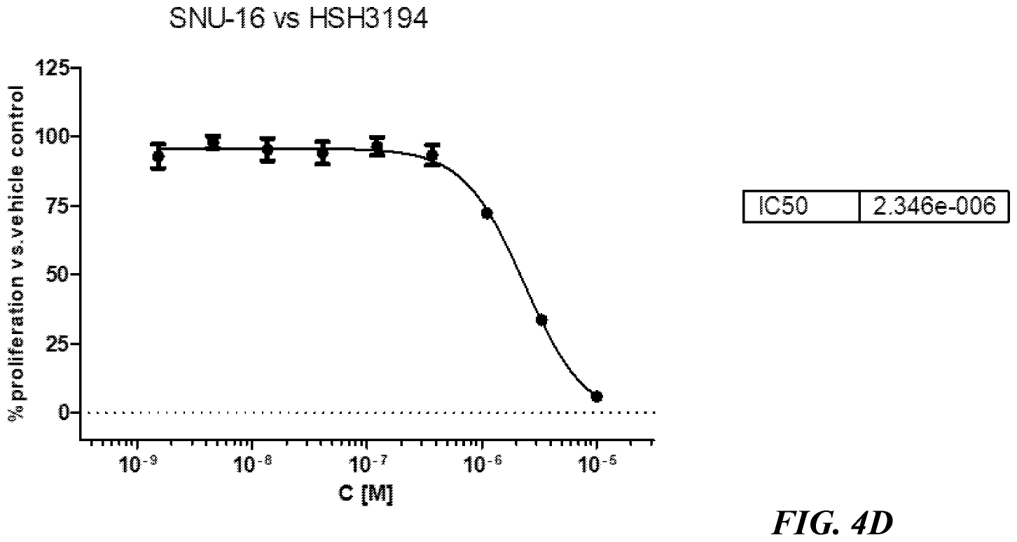

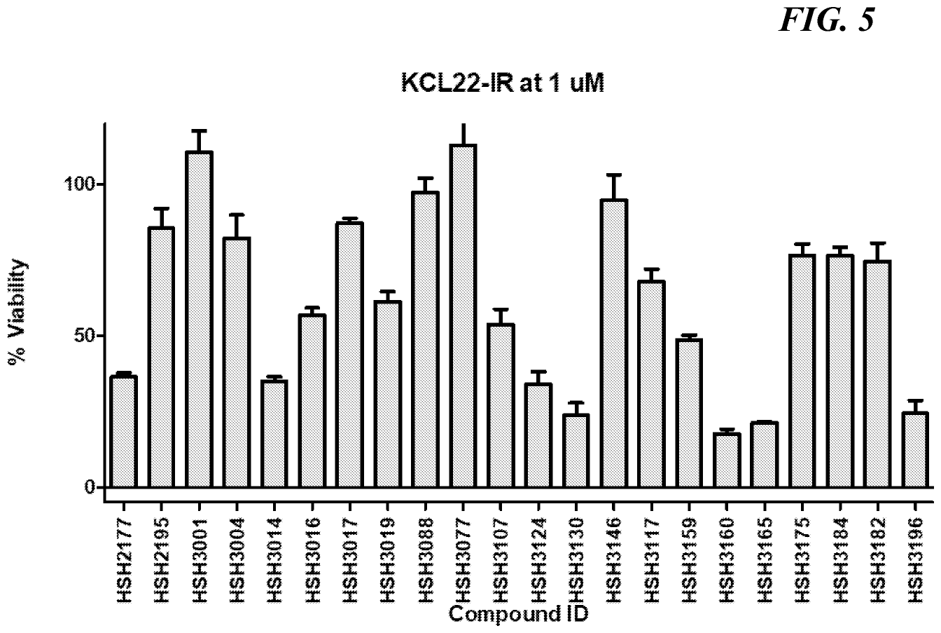

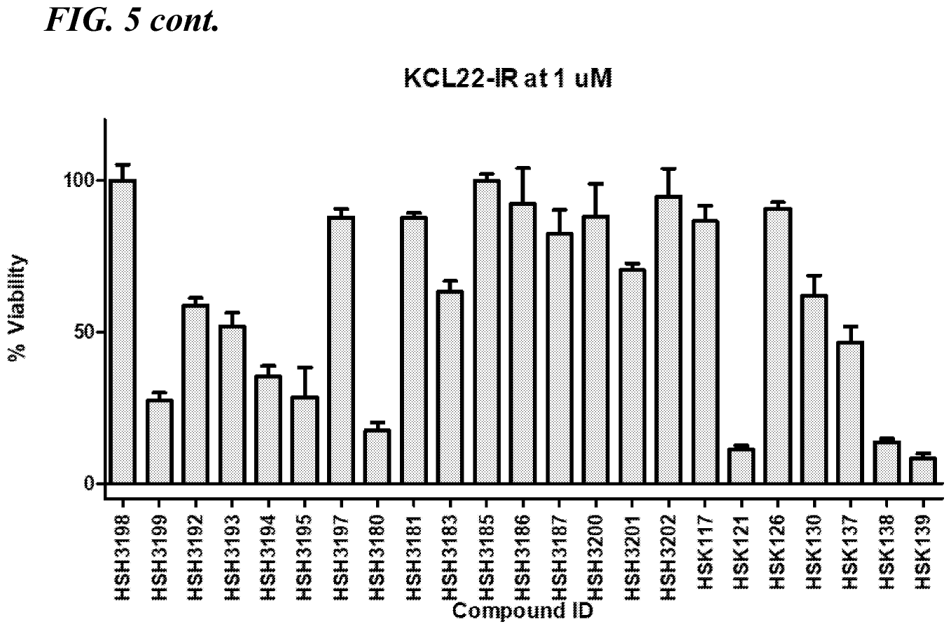

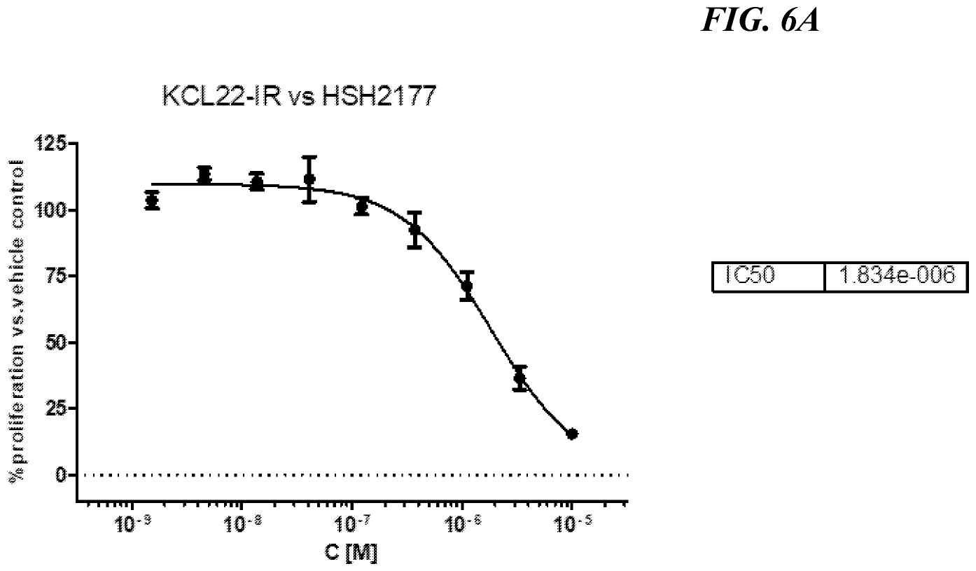

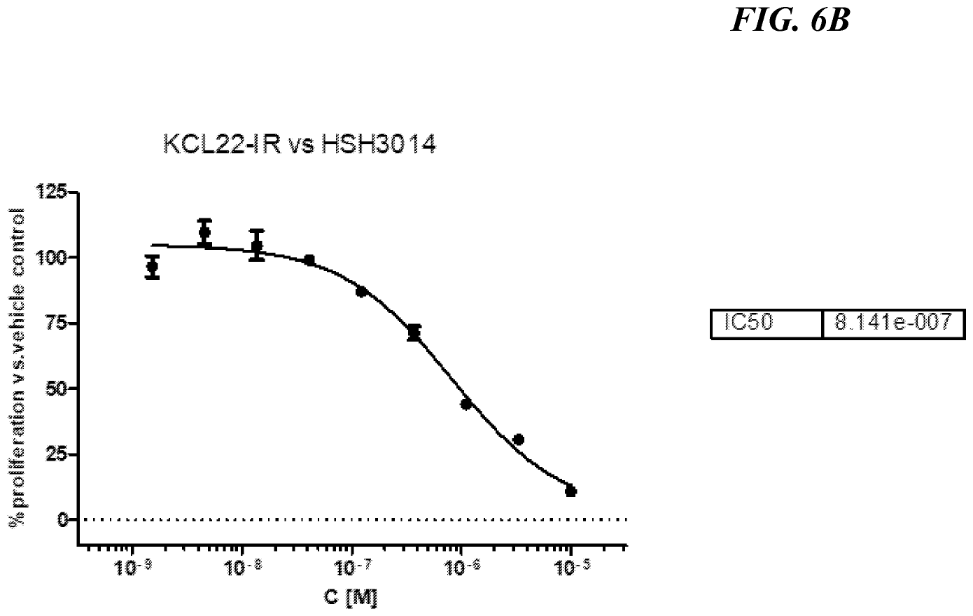

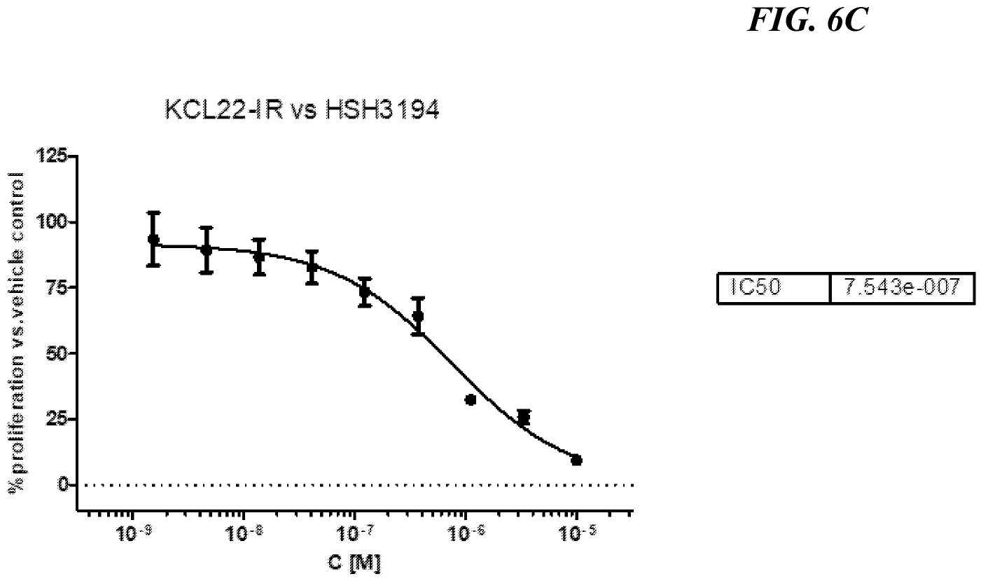

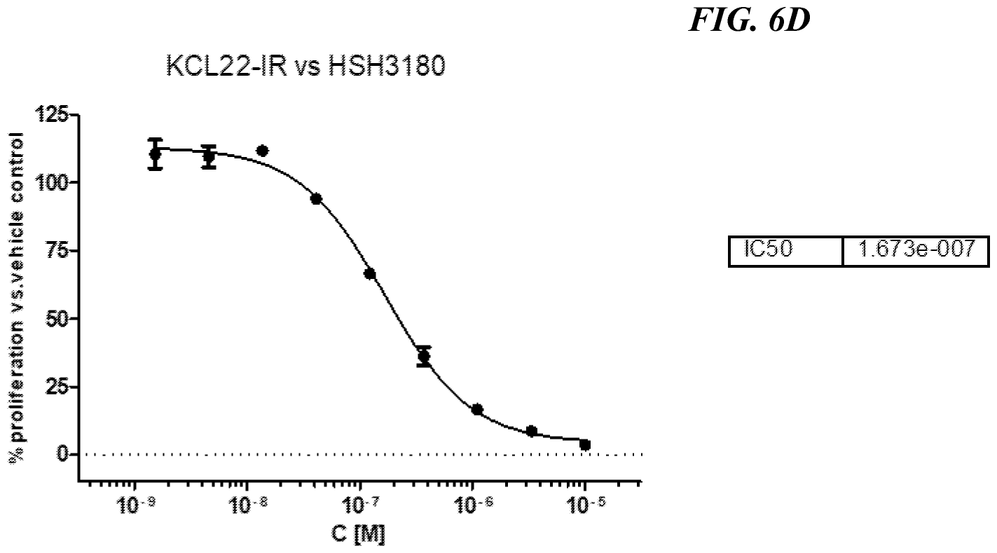

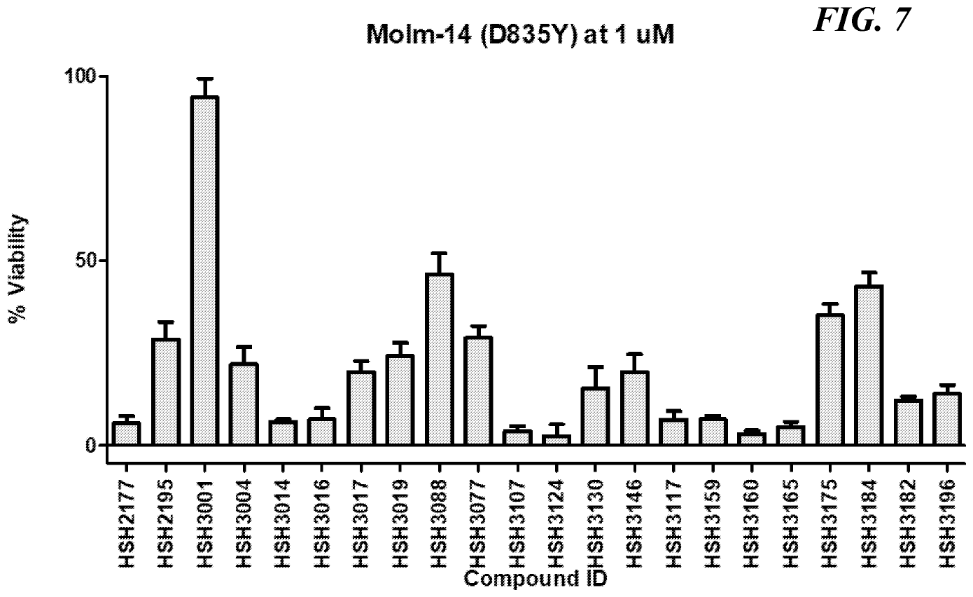

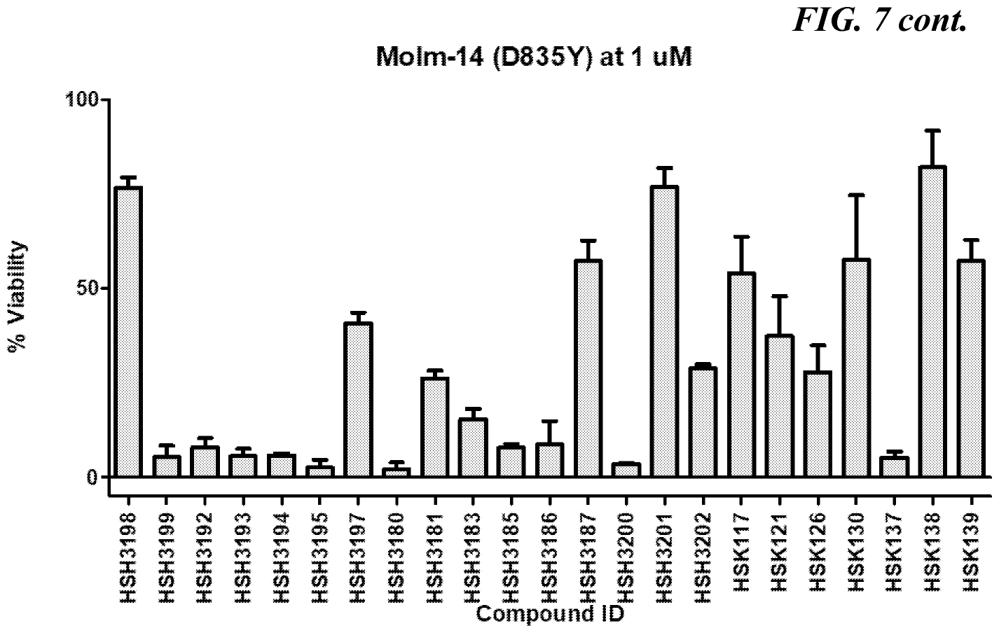

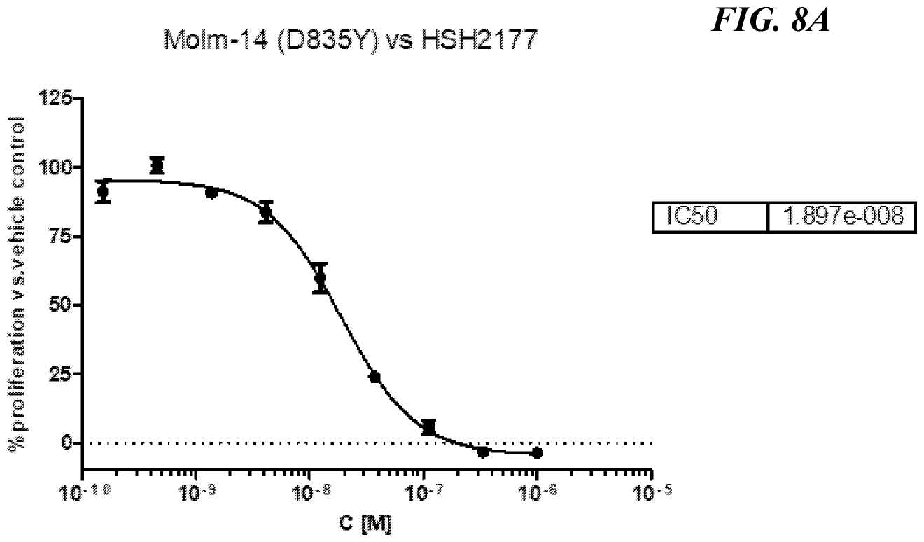

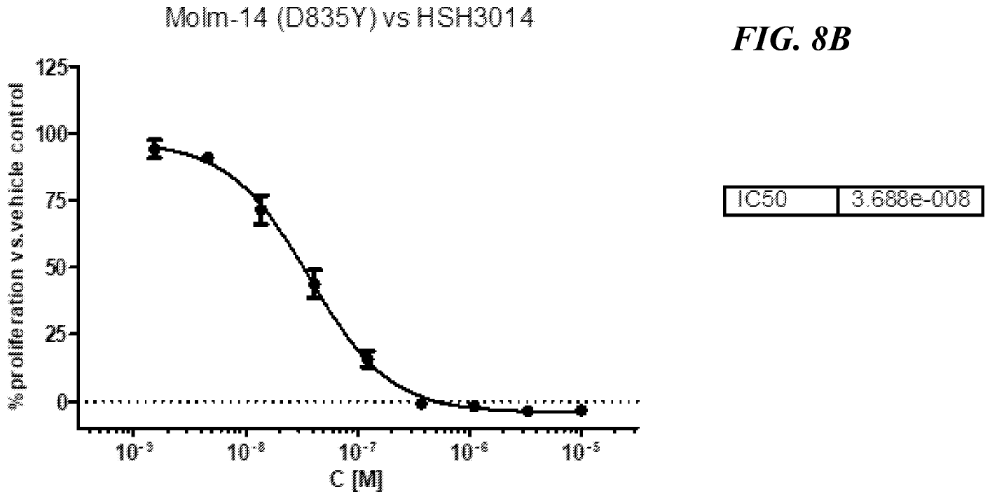

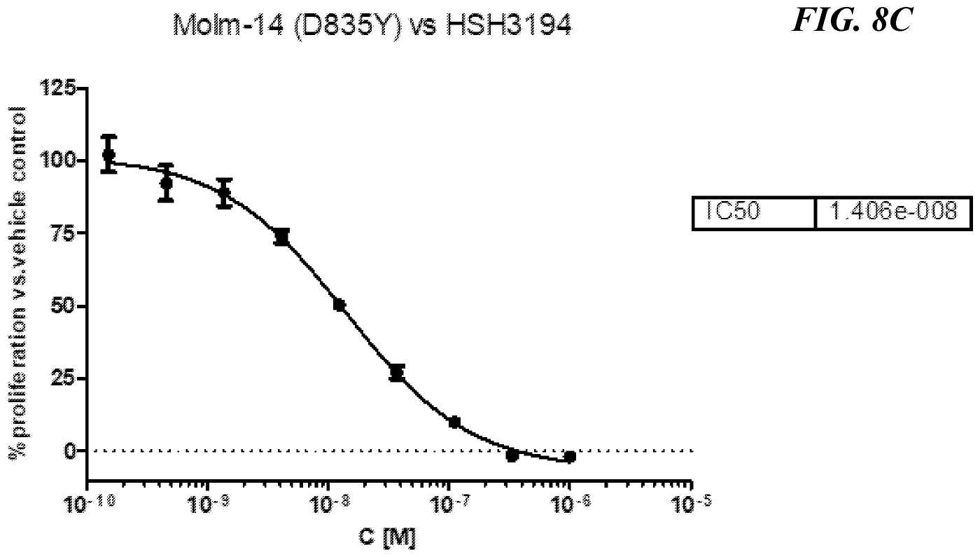

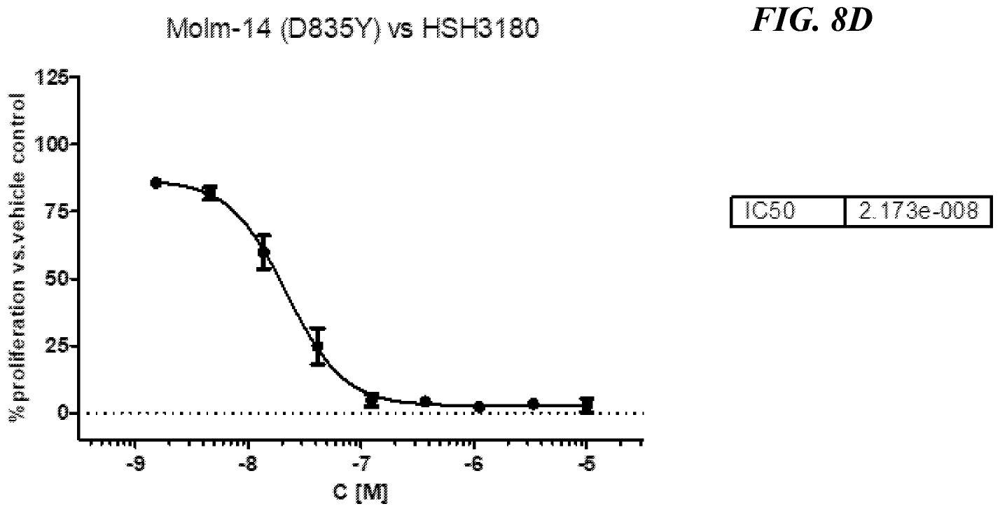

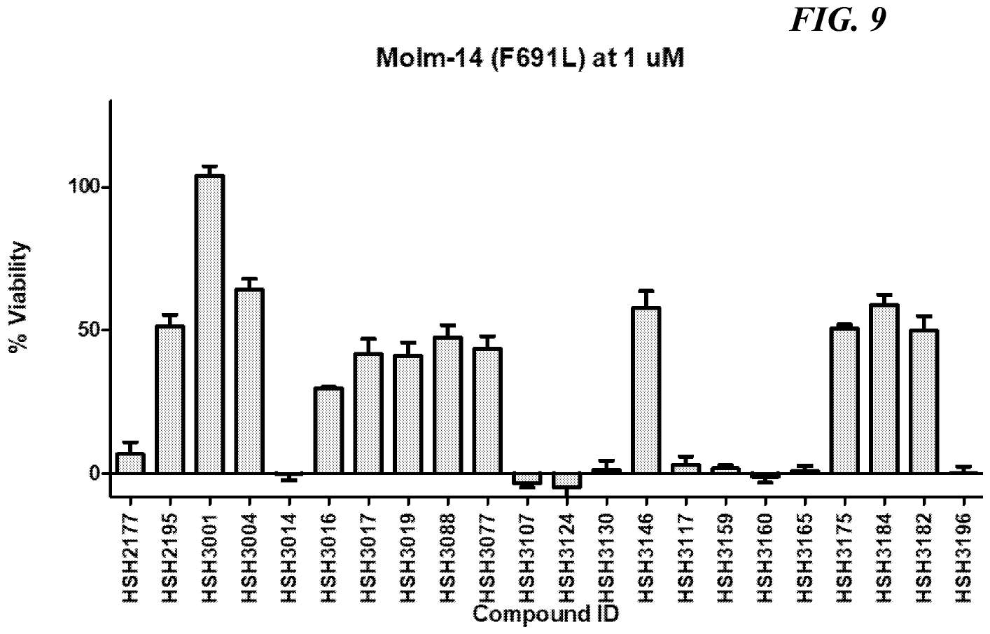

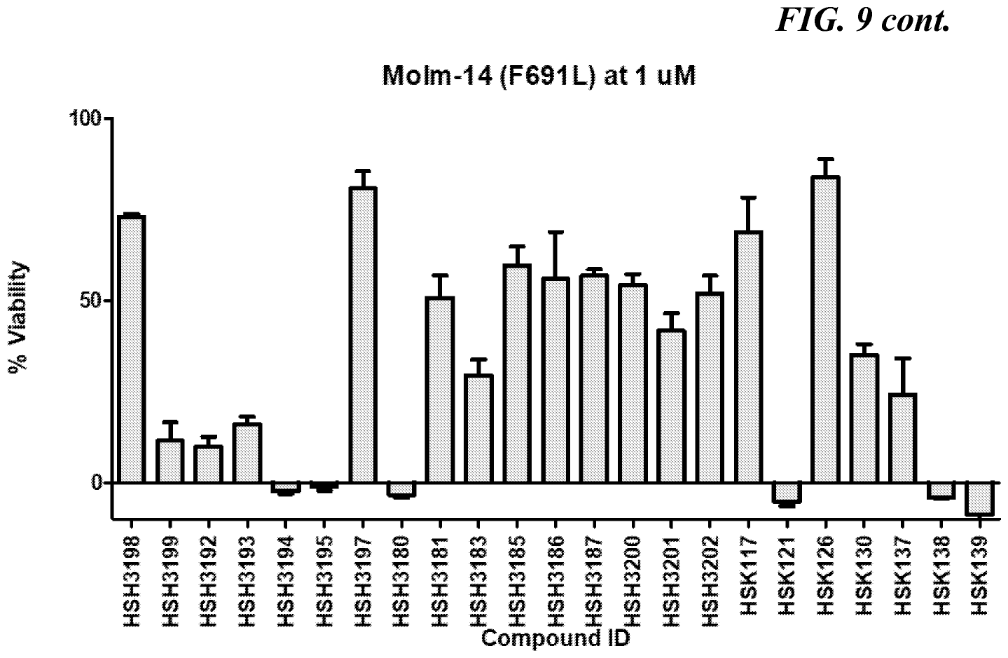

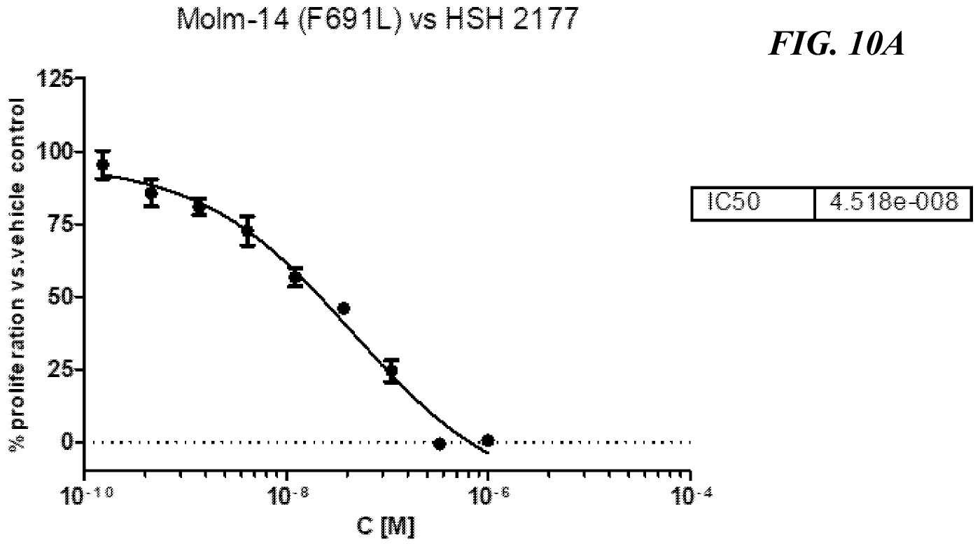

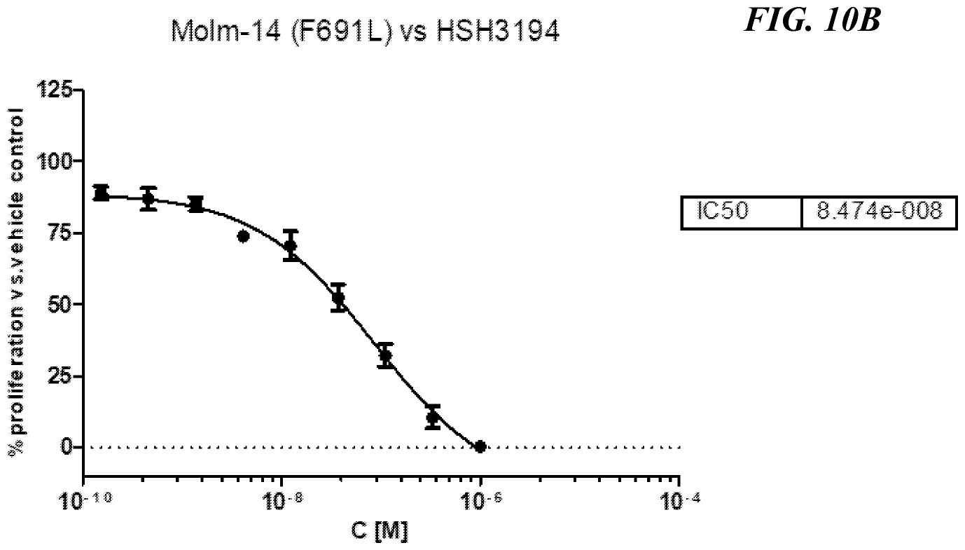

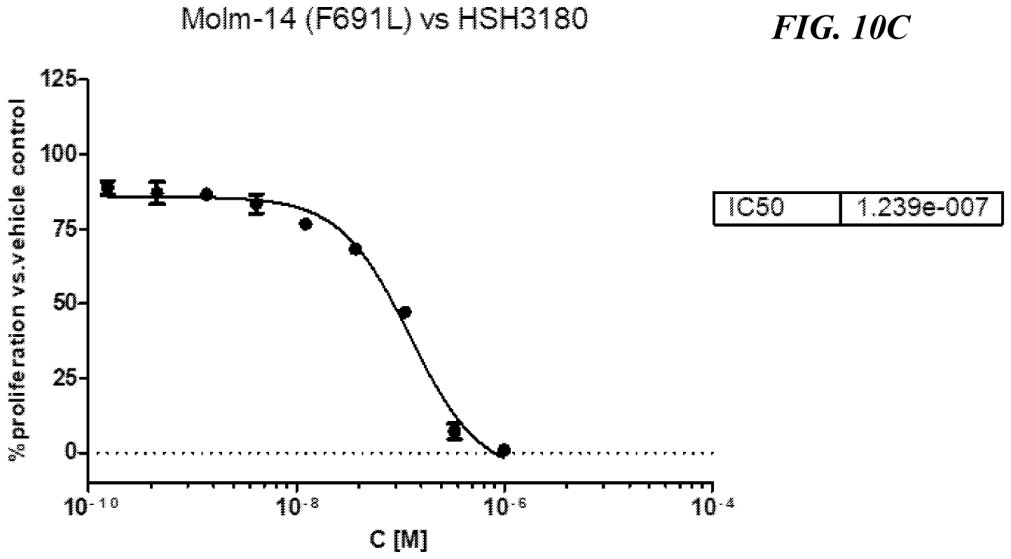

Afterwards, cells were treated with CellTiter-Blue Viability Assay and incubated in a 5% CO₂ incubator for three hours and then the absorbance was measured using the Cytation 5 plate reader at 570 nm. Data was analyzed using GraphPad prism. FIGS. 3-10C show the results.

### Example 3

### Analysis of Docked Structure of Compound HSD1217 to Haspin

To optimize a compound for dual FLT3 and haspin kinase inhibition, docking scores for several of the dual FLT3/haspin inhibitor compounds were assessed. The determination of three-dimensional structures of protein-receptor complexes is an important part of structure-based drug design.

Briefly, the crystal structure for haspin (PDB: 2WB8) was downloaded from the Protein Data Bank and edited to remove any existing bound ligand within the protein active site. Two-dimensional ligands were built in ChemDraw 21.0.0 and were transferred to three-dimensional mol2 files using OpenBabel. Docking was performed using the Glide software by Schrödinger. Waters were removed before docking into haspin. Docking trial validation for the haspin kinase was based off the previously generated docking pose of HSD972. Visualization was performed through PyMol and Maestro.

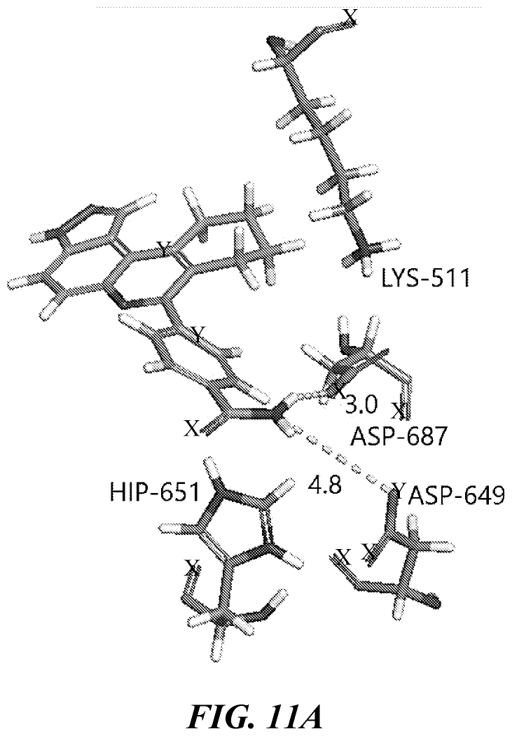

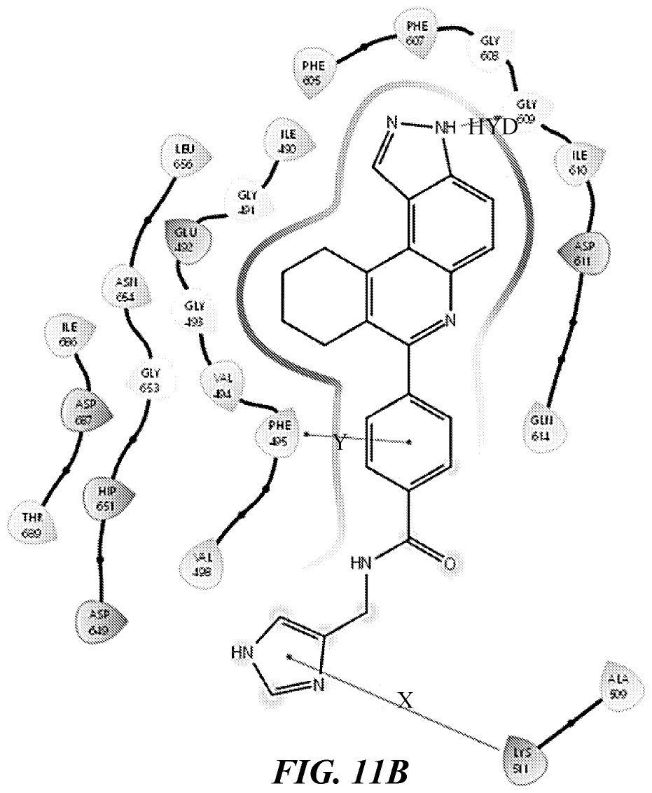

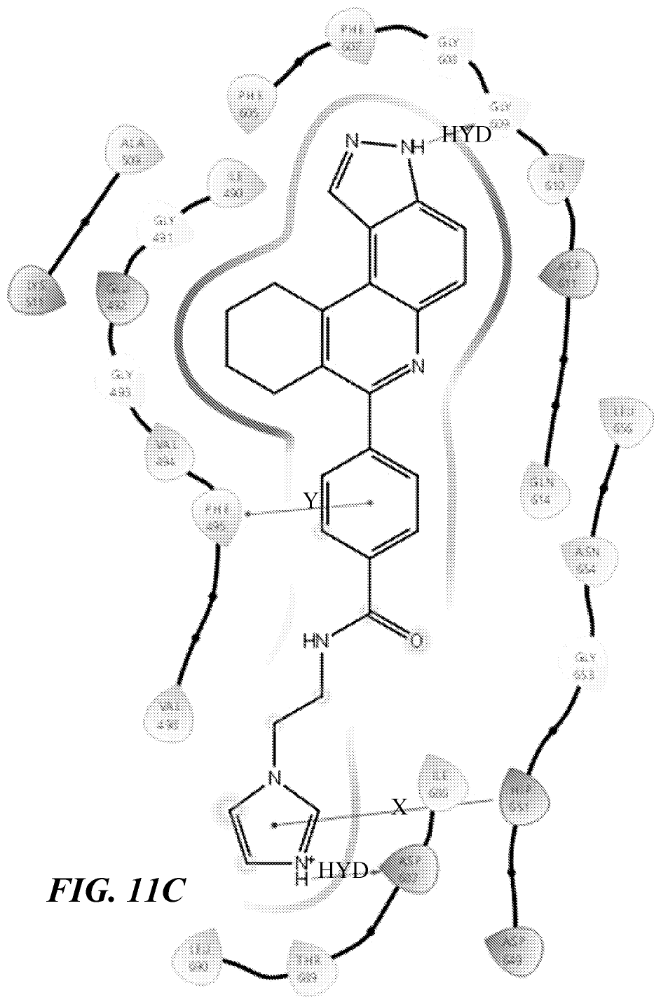

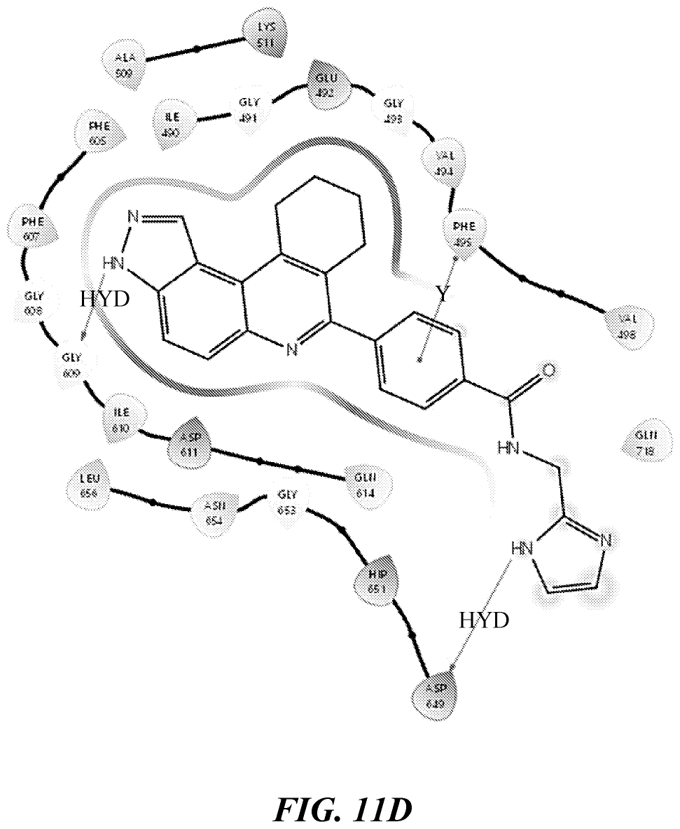

Analysis of the docked structure of Compound HSD1217 to haspin (FIG. 11A) indicated that the benzamide-NH₂ moiety had a hydrogen bonding interaction with key residue D687 and was positioned towards a region harboring H651 and D649 residues, which bind to the ATP phosphate moiety. Thus, appending polar groups, such as imidazole, with hydrogen bonding donating capabilities to the benzamide can reach either H651 or D649 residues while maintaining hydrogen bonding with the D687 residue to provide additional interactions and increase ligand affinity for the protein. Because H651, D649, or D687 are critical for ATP binding to haspin, it was speculated that the inhibitors that engage these residues would have a higher chance of resisting kinase mutations that can abrogate inhibitor binding. Eswaran et al., Structure and functional characterization of the atypical human kinase haspin, Proceedings National Academy Science USA 106 (48): 20198-20203 (2009).

In silico analysis of Compounds 4, 5, and 6 (analogs of Compound HSD1217), which each contain the imidazole moiety, indicated that all three compounds could form key interactions with at least one of these ATP-binding residues (FIGS. 11B-11D). For docking analysis of 3H-pyrazolo[4,3-f]quinoline-based compounds to FLT3, see Dayal et al. (2021), supra.

### Example 4

### Kinase Assay and Structure-Activity Relationship Study

Using the preliminary compounds identified in Example 3, a structure-activity relationship (SAR) study was performed to explore how modifications affect haspin and/or FLT3 inhibition, and new imidazoyl-containing pyrazolo-based compounds (described herein) that are dual FLT3/haspin inhibitors, that inhibit both kinases at ultra-low (sub-nanomolar) concentrations, were identified.

Multicomponent reactions (MCRs) have often been utilized to rapidly synthesize a vast range of highly functionalized analogs in a single pot to build libraries for biological screening. Here, compounds were synthesized using the Doebner-like MCR followed by subsequent amidation (Scheme 3).

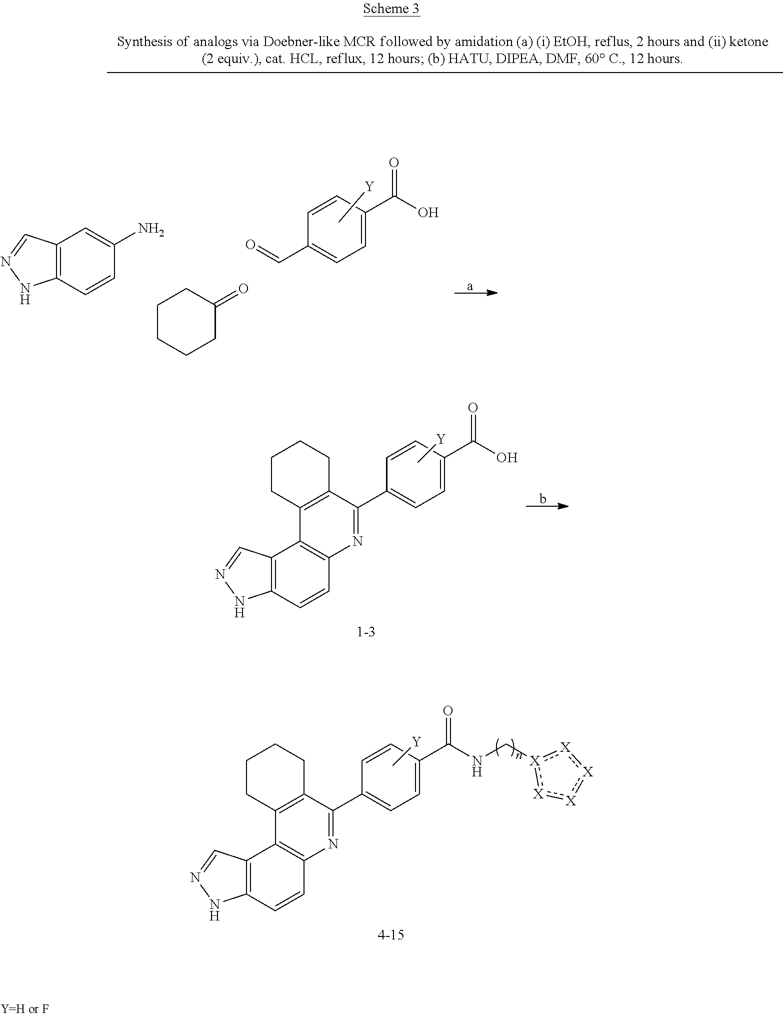

For an initial pilot library, amines bearing imidazole or bioisoteres thereof were the focus as it was thought such heterocyclic amines (which are found in other drugs, such as nilotinib) could potentially facilitate ligand engagement with protein residues, vide supra. During the Doebner-like MCR, the amine and aldehyde react to form the Schiff base, which subsequently reacts with the added ketone in the presence of the acid catalyst (HCl).

With the MCR product A (see Scheme 3) in hand, the final amide products (5-18) were formed via amidation, using commercially available amines, HATU (1-[Bis(dimethylamino)methylene]-1H-1,2,3-triazolo[4,5-b]pyridinium 3-oxide hexafluorophosphate, hexafluorophosphate azabenzotriazole tetramethyl uronium) and Hünig's base (DIPEA).

With the amide kinase inhibitor library established, kinase inhibition and AML cell line (Molm-14, which harbors FLT3-ITD) growth inhibition were analyzed (Table 1). More specifically, Haspin and FLT3 inhibition were evaluated in-house via ADP Glo Kinase Assay (Promega, Madison, WI). All compounds were screened against kinases and Molm-14 cells at a concentration of 50 nM, and HSD1169 and gilteritinib were used as positive controls. Values reported are means of triplicates.

Briefly, the ADP-Glo Kinase Assay Kit was used to determine inhibition of haspin. Kinase Buffer (SignalChem Cat #K01-09; 25 mM MOPS, pH 7.2, 12.5 mM β-glycerol-phosphate, 25 mM MgCl₂, 2 mM EDTA) was supplemented with dithiothreitol (DTT) (0.2 mM) and bovine serum albumin (BSA) (40 μg/mL) immediately prior to use. Reaction volumes of 5 μL containing 9 ng/μL kinase, 10 μM ATP, 50 nM inhibitor, and the appropriate peptide substrate (20 μM) were incubated for 3 hours, and then quenched with an equal volume (5 μL) of GLO reagent. After 40 minutes, 10 μL DET reagent was added and incubated for 1 hour before visualization on a Cytation™ 5 Cell Imaging Multi-Mode Reader (BioTek).

TABLE 1
Structures of compounds evaluated, kinase inhibition, and cellular growth inhibition data.

| | | Percent Inhibition (%) at 50 nM inhibitor conc. | | |
|---|---|---|---|---|
| Compound | Structure | Haspin | FLT3 | Molm-14 |
| 4 | 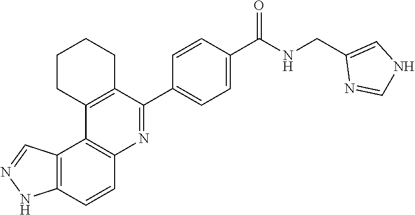 [SMILES: O=C(NCc1c[nH]cn1)c1ccc(-c2nc3ccc4[nH]ncc4c3c3c2CCCC3)cc1] | 54 | 49 | 67 |
| 5 | 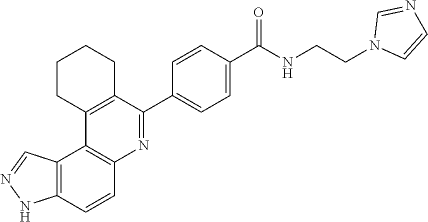 [SMILES: O=C(NCCn1ccnc1)c1ccc(-c2nc3ccc4[nH]ncc4c3c3c2CCCC3)cc1] | 33 | 31 | 42 |
| 6 | 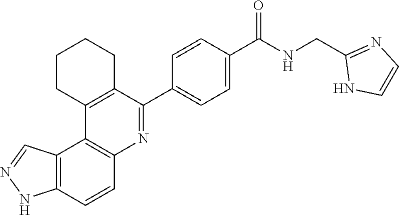 [SMILES: O=C(NCc1ncc[nH]1)c1ccc(-c2nc3ccc4[nH]ncc4c3c3c2CCCC3)cc1] | 77 | 76 | 84 |
| 7 | 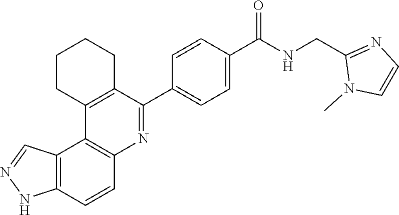 [SMILES: Cn1ccnc1CNC(=O)c1ccc(-c2nc3ccc4[nH]ncc4c3c3c2CCCC3)cc1] | 26 | 14 | 21 |
| 8 | 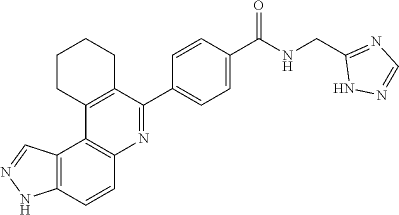 [SMILES: O=C(NCc1ncn[nH]1)c1ccc(-c2nc3ccc4[nH]ncc4c3c3c2CCCC3)cc1] | 57 | 49 | 48 |
| 9 | 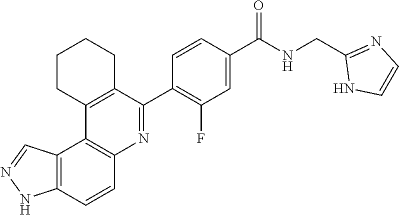 [SMILES: O=C(NCc1ncc[nH]1)c1ccc(-c2nc3ccc4[nH]ncc4c3c3c2CCCC3)c(F)c1] | 53 | 88 | 87 |
| 10 | 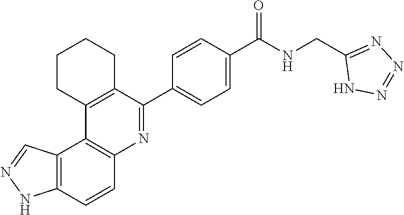 [SMILES: O=C(NCc1nnn[nH]1)c1ccc(-c2nc3ccc4[nH]ncc4c3c3c2CCCC3)cc1] | 57 | 51 | 50 |
| 11 | 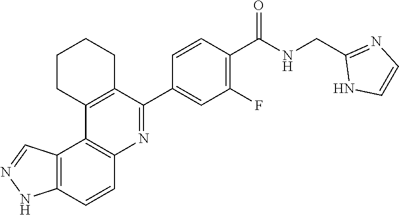 [SMILES: O=C(NCc1ncc[nH]1)c1ccc(-c2nc3ccc4[nH]ncc4c3c3c2CCCC3)cc1F] | 76 | 85 | 80 |
| 12 | 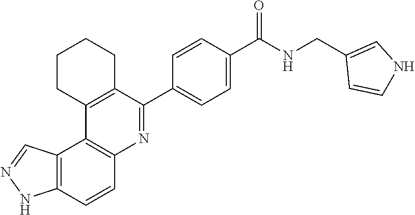 [SMILES: O=C(NCc1cc[nH]c1)c1ccc(-c2nc3ccc4[nH]ncc4c3c3c2CCCC3)cc1] | 41 | 46 | 39 |
| 13 | 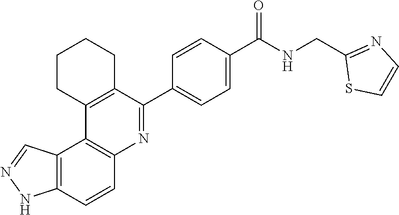 [SMILES: O=C(NCc1nccs1)c1ccc(-c2nc3ccc4[nH]ncc4c3c3c2CCCC3)cc1] | 68 | 53 | 55 |
| 14 (HSK205) | 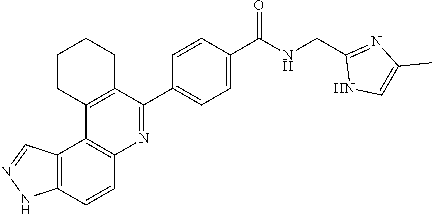 [SMILES: Cc1c[nH]c(CNC(=O)c2ccc(-c3nc4ccc5[nH]ncc5c4c4c3CCCC4)cc2)n1] | 97 | 94 | 96 |
| 15 | 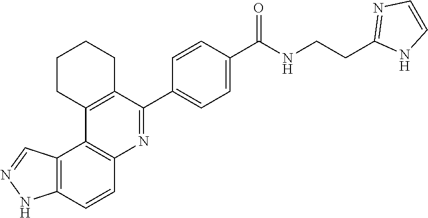 [SMILES: O=C(NCCc1ncc[nH]1)c1ccc(-c2nc3ccc4[nH]ncc4c3c3c2CCCC3)cc1] | 78 | 71 | 73 |
| HSD1169 | 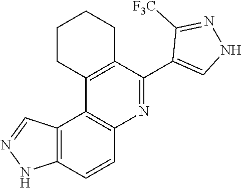 [SMILES: FC(F)(F)c1n[nH]cc1-c1nc2ccc3[nH]ncc3c2c2c1CCCC2] | 39 | 81 | 90 |
| Gilteritinib | 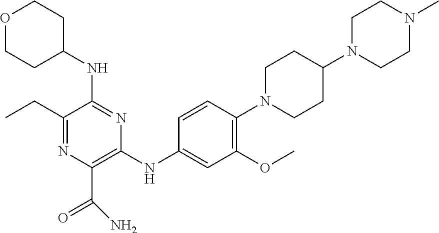 [SMILES: CCc1nc(C(N)=O)c(Nc2ccc(N3CCC(N4CCN(C)CC4)CC3)c(OC)c2)nc1NC1CCOCC1] | 11 | 93 | 87 |

The SAR study supported that the imidazole ring is critical for activity against both haspin and FLT3, as even subtle modifications dramatically affected kinase inhibition. Compounds 4, 5 and 6 all contain the imidazole moiety, albeit substituted differently; their inhibition profile against the tested kinases are however different.

Compound 6, the 2-substituted imidazole, inhibited haspin and FLT3 (77% and 76%, respectively) better than Compound 5, the 1-substituted imidazole, (33% and 31%, respectively), and Compound 4, the 4-substituted imidazole (54% and 49%, respectively).

As Compound 6 showed the best inhibitions of the tested kinases, substitution patterns on Compound 6 were explored to potentially improve potency. Substituting the —NH on Compound 6 with —NMe, as in Compound 7, reduced haspin and FLT3 inhibition as well as AML growth inhibition. For example, while at 50 nM, Compound 6 inhibited haspin at 77%, the —NMe analog (Compound 7) inhibited the same enzyme at 26%. For FLT3, potency dropped from 76% inhibition with Compound 6 at 50 nM to only 14% inhibition with Compound 7. Replacing the imidazole unit in Compound 6 with other 5-membered bioisosteres (triazole (Compound 8), tetrazole (Compound 10) or thiazole (Compound 13)) afforded compounds that could inhibit both kinases and AML cell proliferation, but generally less so than Compound 6. Extending the length of carbon chain between the amide and the imidazole by one unit to give Compound 15 did not drastically impact inhibition of either kinase.

The effects of fluorine substitution on lead compounds are routinely explored as it is known that fluorine can be used to tune drug properties (both target engagement as well as general drug properties, such as metabolic stability). Accordingly, fluorinating the benzamide moiety of Compound 6 to afford Compounds 9 and 11 was performed; however, FLT3 and haspin kinase inhibitions were not significantly impacted (compare Compounds 9 and 11 with Compound 6, Table 1).

Some imidazole-containing drugs, such as nilotinib, have the imidazole unit methylated at position 4. Thus, the extent that methylation of the imidazole unit in Compound 6 would affect kinase inhibition was also investigated. Methylation of the imidazole unit significantly improved potency against haspin, FLT3, and Molm-14 cell proliferation. Compound 14 (also called Compound HSK205) inhibited haspin and FLT3 at 97% and 94%, respectively, at only 50 nM, supporting that Compound HSK205 is an ultra-potent inhibitor of both FLT3 and haspin (vide infra). Indeed, at a concentration of only 50 nM, Compound HSK205 almost completely inhibited (96%) the growth of Molm-14 (FLT3-ITD) cells.

To validate the laboratory results, Compound HSK205 was sent to contract research organizations, Eurofins and Reaction Biology, to obtain binding data for haspin (Eurofins) and enzyme inhibition data for FLT3 (Reaction Biology).

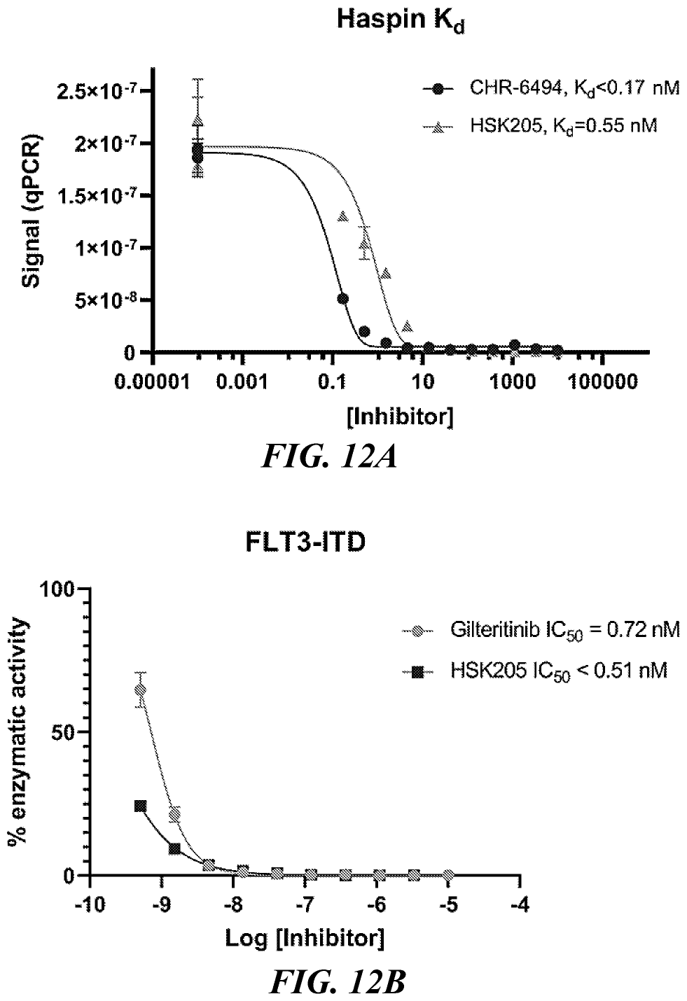

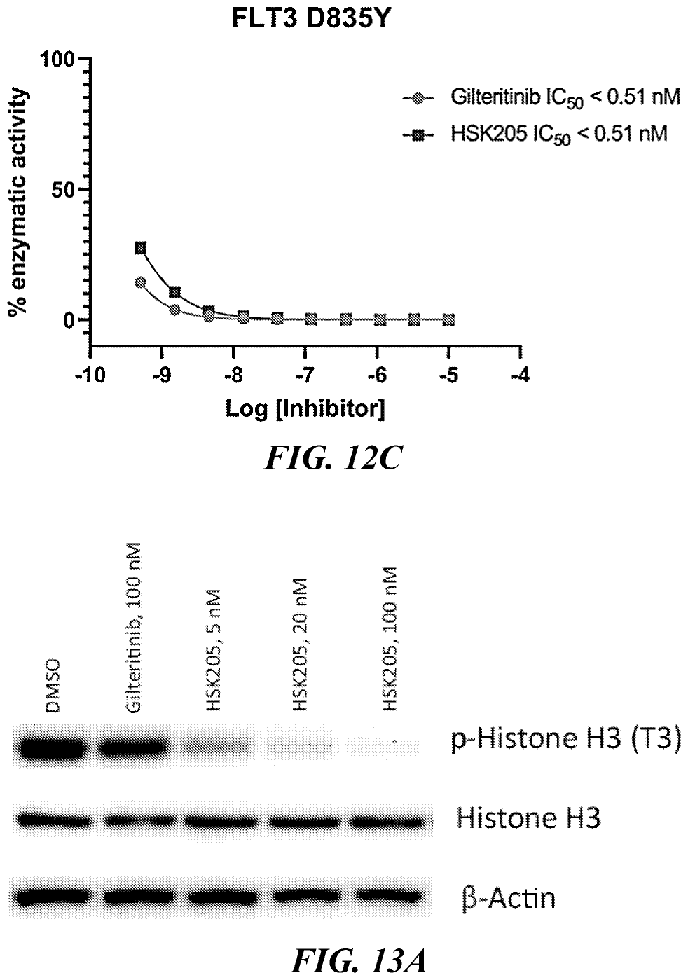

Compound HSK205 bound to haspin with K_{d}=0.55 nM (K_{d} of control haspin inhibitor CHR-6494<0.17 nM) (FIG. 12A).

With regards to FLT3 inhibition, at 10 μM ATP, Compound HSK205 has an IC₅₀<0.5 nM for both FLT3-ITD and FLT3-D835Y, while under similar conditions, gilteritinib had IC_{50=0.7} nM and IC₅₀<0.5 nM, respectively (FIGS. 12B-12C).

These SAR studies validated earlier reports that the 3H-pyrazolo[4,3-f]quinoline moiety can target FLT3 and also that the moiety is highly tunable, in that substitution at the C₇ position can be used to target other kinases. For example, Compound HSD1169, also a 3H-pyrazolo[4,3-f]quinoline-containing compound and bearing a fluorinated pyrazole is a strong inhibitor of FLT3 but a weak inhibitor of haspin, while changing the C₇ moiety to a benzamide leads to a dual FLT3/haspin inhibitor.

### Example 5

### Target Engagement and In Vitro Biological Activity

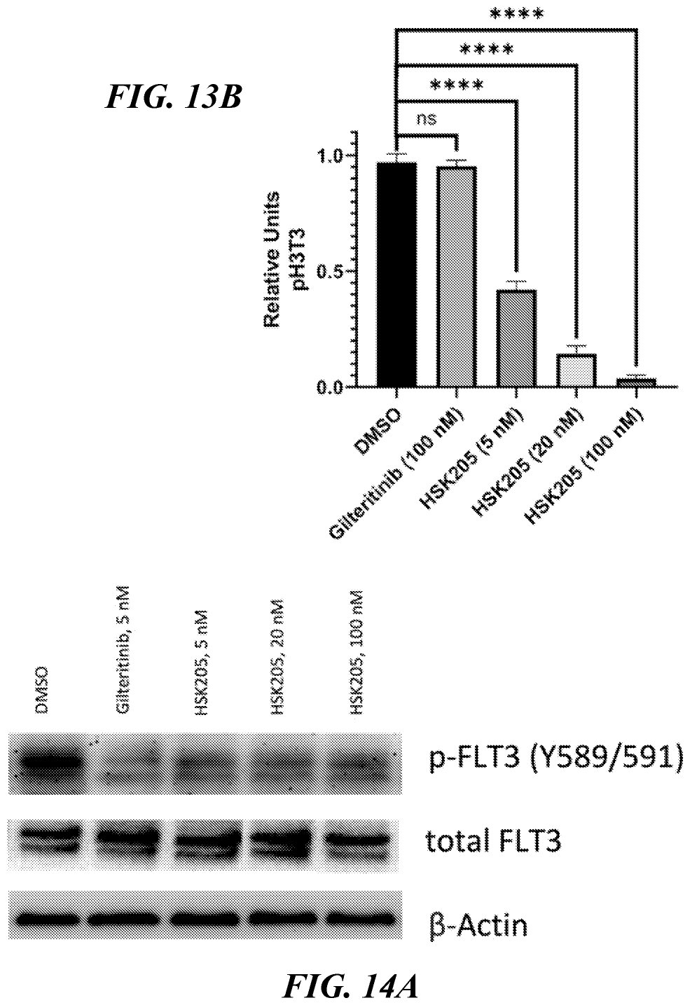

Whether Compound HSK205 does indeed inhibit haspin in AML cells was then evaluated, and immunoblotting was performed to confirm inhibition of histone H3 (Thr3)phosphorylation by haspin (FIG. 13A). Briefly, Molm-14 cells were treated with either Compound HSK205 (5 nM or 20 nM or 100 nM) or a DMSO control or gilteritinib (a negative control) at the higher 100 nM concentration, and cells were harvested after 24 hours of treatment. The samples were subsequently analyzed for the phosphorylation of histone H3 (Thr3).

More specifically, cells were cultured to confluence in RPMI medium supplemented with 10% fetal bovine serum and 1× Penicillin-Streptomycin (Gibco™). Cells were plated at 5,000 cells/well in 96 well plates. After 24-hour incubation at 37° C. in a 5% CO₂ incubator, compounds diluted in the same media were added to the indicated final concentration and allowed to incubate at 37° C. in a 5% CO₂ incubator for 72 hours. Viability was determined using CellTiter-Blue® Cell Viability Assay (Promega, Madison, WI) with absorbance readings at 570 nm performed on a Cytation™ 5 Cell Imaging Multi-Mode Reader (BioTek) 3 hours after addition. Appropriate vehicle and cell-free controls were included. GI₅₀ readings were confirmed by two scientists on two separate days.

Acute myeloid leukemia cells (Molm-14) were incubated with various concentrations of compound as specified. Cells were collected and lysed, and protein concentrations were quantified relative to BSA standards. After protein denaturation, proteins were separated via SDS-PAGE electrophoresis and transferred to NC membranes. After blocking, overnight incubation with specific primary antibodies, and room temperature incubation for 2 hours with peroxidase-conjugated secondary antibodies, peroxidase activity was detected with SuperSignal West Pico reagents (Thermo Scientific) using a CCD camera LAS-4000 (Fujifilm). Bands were normalized using housekeeping gene, Actin. Values reported represent the means of triplicates and error bars represent SD. All specific antibodies were purchased from Cell Signaling.

The results indicated that Compound HSK205 was effective in reducing phosphyorlation of histone H3 (Thr3), with a more pronounced effect at 20 nM as compared to the 5 nM and 100 nM compared to 20 nM (FIG. 13B). Gilteritinib, however, did not display haspin inhibition in Molm-13 cells.

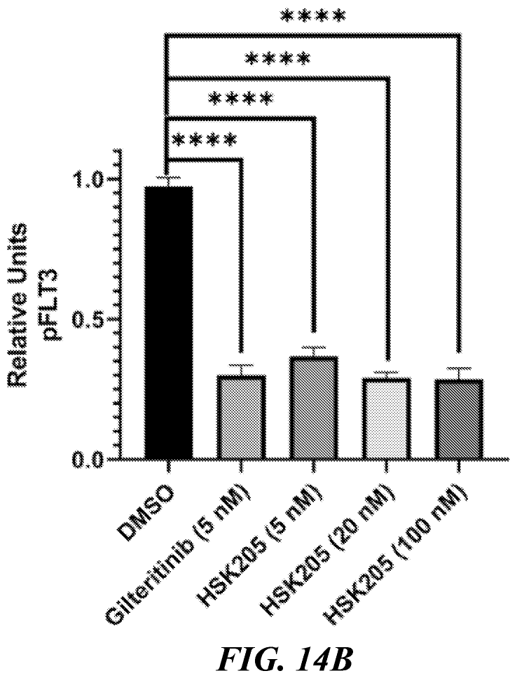

Additionally, FLT3 target engagement in Molm-14 cells was evaluated to confirm the FLT3 inhibition seen in the preliminary kinase screen of Example 4 (Table 1). Cells were treated for 2 hours, harvested, and subsequently analyzed. Administration of Compound HSK205 to Molm-14 cells led to a reduction of phosphorylated FLT3 (FIGS. 14A and 14B).

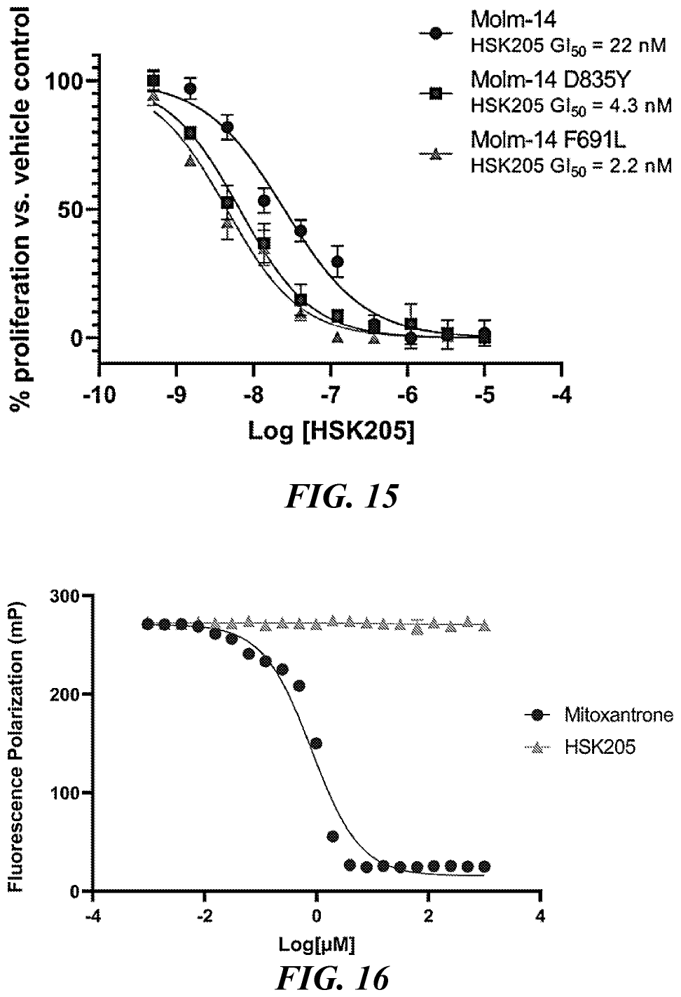

Many FLT3 inhibitors have been trialed in the clinic and some, such as gilteritinib, midostaurin and quizartinib, have been approved; however, on-target FLT mutations that reduce AML sensitivity to these drugs have been observed. Scholl et al. (2020), supra. Accordingly, Compound HSK205 was evaluated against Molm-14 cell lines that harbor resistant mutations commonly found in the clinic, FLT3-ITD-D835Y and F691L (FIG. 15). Remarkably, Compound HSK205 showed low nanomolar GI₅₀ values against Molm-14-ITD-F691L (2.2 nM) and Molm 14-ITD-D835Y (4.3 nM).

To move compounds forward for further development, DNA intercalation assays were conducted to eliminate compounds with high affinity for DNA. Although Compound HSK205 contains polycyclic rings, which can enhance DNA intercalation and unwanted toxicity, Compound HSK205 did not intercalate into DNA (FIG. 16). Under similar experimental conditions, mitoxantrone (a known DNA intercalator) did associate with DNA (FIG. 16).

### Example 6

### Chemistry and Synthesis of Compound HSK205 and Analogs Thereof

For Examples 3-5, unless noted otherwise, all reagents and solvents were purchased from commercial sources and used as received. The 1H and ¹³C NMR spectra were obtained in DMSO-d₆ as solvent using a 500 MHz spectrometer or 800 MHz spectrometer with Me4Si as an internal standard. Chemical shifts are reported in parts per million (8) and were calibrated using residual un-deuterated solvent as an internal reference.

Data for ¹H NMR spectra are reported as follows: chemical shift (8 ppm) (multiplicity, coupling constant (Hz), integration). Multiplicities are reported as follows: s=singlet, d=doublet, t=triplet, q=quartet, m=multiplet, or combinations thereof. High resolution mass spectra (HRMS) were obtained using electron spray ionization (ESI) technique and a TOF mass analyzer. New compounds were characterized by ¹H NMR, ¹³C NMR, and HRMS data, and purities of final compounds were reported using HPLC.

### 4-(8,9,10,11-tetrahydro-3HI-pyrazolo[4,3-a]phenanthridin-7-yl)benzoic acid (Compound 1)

In a round bottom flask equipped with a stir bar, the appropriate indazole (1 eq) and the appropriate aldehyde (1 eq) were dissolved in ethanol. This mixture was refluxed at 120° C. for 2 hours, after which the appropriate ketone (2 eq) and a catalytic amount of hydrochloric acid was added. This was allowed to stir at reflux for an additional 12 hours. The reaction solvent was evaporated using a rotary evaporator and purified via column chromatography with 10% methanol in ethyl acetate to yield Compound 1 as an off-white solid (2.38 g, 69.4%).

¹H NMR (500 MHZ, (CD₃)₂SO) δ 8.75 (s, 1H), 8.26 (d, J=9.2 Hz, 1H), 8.19 (d, J=9.2 Hz, 1H), 8.15-8.11 (m, 2H), 7.85-7.80 (m, 2H), 3.46 (t, J=6.5 Hz, 2H), 2.76 (t, J=6.1 Hz, 2H), 2.03 (dd, J=6.0, 2.9 Hz, 2H), 1.83-1.76 (m, 2H). ¹³C NMR (126 MHZ, (CD₃)₂SO) δ 167.17, 151.31, 151.03, 140.41, 138.09, 137.05, 134.78, 132.69, 131.32, 130.42, 129.69, 123.45, 122.02, 120.11, 115.06, 30.67, 28.05, 21.85, 21.72. HRMS (ESI) m/z calcd for C₂₁H₁₇N₃O₂ [M+H]⁺344.1393, found 344.1392.

### 3-fluoro-4-(8,9,10,11-tetrahydro-3H-pyrazolo[4,3-a]phenanthridin-7-yl)benzoic acid (Compound 2)

Following the same synthetic procedure as Compound 1, Compound 2 was obtained as an off-white solid (636 mg, 58.7%). ¹H NMR (500 MHZ, (CD₃)₂SO) δ 8.64 (s, 1H), 8.06 (d, J=22.2 Hz, 3H), 7.72 (d, J=11.2 Hz, 1H), 7.63 (d, J=8.1 Hz, 1H), 3.31 (t, J=6.6 Hz, 2H), 2.76 (t, J=6.2 Hz, 2H), 2.04-1.91 (m, 2H), 1.76 (q, J=6.0 Hz, 2H). ¹³C NMR (126 MHZ, (CD₃)₂SO) δ 167.55, 165.09, 162.00, 159.95, 157.23, 150.10, 132.37, 131.56, 130.83, 126.27, 123.27, 123.14, 121.70, 120.78, 120.70, 118.99 (2J=22 Hz), 115.01, 30.42, 28.01, 21.88, 21.75. HRMS (ESI) m/z calcd for C₂₁H₁₆FN₃O₂ [M+H]⁺362.1299, found 362.1300.

### 2-fluoro-4-(8,9,10,11-tetrahydro-3HI-pyrazolo[4,3-a]phenanthridin-7-yl)benzoic acid (Compound 3)

Following the same synthetic procedure as Compound 1, Compound 3 was obtained as an off-white solid (556 mg, 51.3%). ¹H NMR (500 MHZ, (CD₃)₂SO) δ 8.76 (d, J=8.6 Hz, 1H), 8.16 (d, J=14.3 Hz, 2H), 8.04 (t, J=7.7 Hz, 1H), 7.67 (d, J=11.1 Hz, 1H), 7.60 (d, J=8.1 Hz, 1H), 3.47 (t, J=6.6 Hz, 2H), 2.80 (t, J=6.2 Hz, 2H), 2.05 (p, J=6.0 Hz, 2H), 1.81 (p, J=5.8 Hz, 2H). ¹³C NMR (126 MHz, (CD₃)₂SO) & 165.04, 162.04, 159.99, 150.59, 140.43, 138.54, 134.91, 132.33, 130.91, 126.13, 123.73, 123.41, 120.87, 119.44, 118.75 (2J=20 Hz), 115.32, 109.16, 30.48, 28.04, 21.99, 21.86. HRMS (ESI) m/z calcd for C₂₁H₁₆FN₃O₂ [M+H]⁺362.1299, found 362.1301.

### N-((1H-imidazol-4-yl)methyl)-4-(8,9,10,11-tetrahydro-3H-pyrazolo[4,3-a]phenanthridin-7-yl)benzamide (Compound 4)

In a 20 mL screw-capped vial equipped with a stir bar, the acid intermediate (1.3 eq) and HATU (1.5 eq) were sealed with a septum, air was removed via a vacuum pump, and a nitrogen balloon was added to the reaction vessel to fill the vessel with an inert gas. To this reaction vial, anhydrous DMF (2 mL) and DIPEA (8 eq) were added. This was heated at 60° C. for 30 minutes, and then the amine (1 eq) was added. The reaction was allowed to stir at 60° C. overnight.

The reaction solvent was evaporated using a rotary evaporator and purified via column chromatography using 1% ammonium hydroxide, 9% methanol and 90% ethyl acetate to afford Compound 4 as an off-white solid (111 mg, 52.6%).

¹H NMR (500 MHZ, (CD₃)₂SO) δ 8.81 (t, J=5.6 Hz, 1H), 8.57 (s, 1H), 7.99 (d, J=8.2 Hz, 2H), 7.94-7.76 (m, 2H), 7.67-7.63 (m, 2H), 7.62 (s, 1H), 6.98 (s, 1H), 4.46 (d, J=5.6 Hz, 2H), 3.32 (t, J=6.5 Hz, 2H), 2.76 (t, J=6.1 Hz, 2H), 2.03-1.95 (m, 2H), 1.74 (dp, J=9.1, 3.2, 2.6 Hz, 2H). ¹³C NMR (126 MHZ, (CD₃)₂SO) δ 166.39, 156.17, 144.01, 143.85, 142.53, 135.85, 135.21, 134.22, 129.65, 129.56, 129.42, 129.24, 127.47, 122.11, 117.09, 116.30, 36.81, 29.65, 28.83, 22.58, 22.50. HRMS (ESI) m/z calcd for C₂₅H₂₂N₆O [M+H]⁺423.1928, found 423.1911.

### N-(2-(1H-imidazol-1-yl)ethyl)-4-(8,9,10,11-tetrahydro-3H-pyrazolo[4,3-a]phenanthridin-7-yl)benzamide (Compound 5)

Following the same synthetic procedure as Compound 4, Compound 5 was obtained as an off-white solid (84 mg, 38.5%). ¹H NMR (800 MHZ, (CD₃)₂SO) δ 8.74 (t, J=5.5 Hz, 1H), 8.57 (s, 1H), 7.93 (d, J=7.8 Hz, 2H), 7.89 (d, J=8.9 Hz, 1H), 7.86-7.80 (m, 1H), 7.65 (d, J=7.8 Hz, 2H), 7.63 (s, 1H), 7.18 (s, 1H), 6.89 (s, 1H), 4.22 (t, J=6.2 Hz, 2H), 3.63 (q, J=5.9 Hz, 2H), 2.78 (t, J=6.2 Hz, 2H), 2.06-1.99 (m, 2H), 1.80-1.73 (m, 2H). ¹³C NMR (201 MHz, (CD₃)₂SO) δ 166.82, 156.07, 144.98, 144.06, 142.49, 138.75, 137.79, 136.28, 133.87, 129.51, 129.39, 128.73, 127.30, 126.85, 119.97, 118.38, 116.37, 114.52, 45.60, 40.91, 29.60, 28.76, 22.50, 22.41. HRMS (ESI) m/z calcd for C₂₆H₂₄N₆O [M+H]⁺437.2084, found 437.2081.

### N-((1HI-imidazol-2-yl)methyl)-4-(8,9,10,11-tetrahydro-3H-pyrazolo[4,3-a]phenanthridin-7-yl)benzamide (Compound 6)

Following the same synthetic procedure as Compound 4, Compound 6 was obtained as an off-white solid (65 mg, 30.8%). ¹H NMR (800 MHZ, (CD₃)₂SO) δ 9.17 (t, J=5.9 Hz, 1H), 8.57 (s, 1H), 8.07 (d, J=7.8 Hz, 2H), 7.89 (d, J=9.0 Hz, 1H), 7.82 (d, J=9.3 Hz, 1H), 7.65 (d, J=7.8 Hz, 2H), 6.93 (s, 2H), 4.56 (d, J=5.6 Hz, 2H), 2.77 (t, J=6.3 Hz, 2H), 2.00 (q, J=6.8, 6.3 Hz, 2H), 1.75 (p, J=5.6 Hz, 2H). ¹³C NMR (201 MHZ, (CD₃)₂SO) δ 166.48, 156.04, 146.73, 145.52, 144.05, 143.70, 142.46, 138.80, 136.15, 133.75, 129.47, 129.33, 129.16, 127.57, 127.50, 122.02, 119.08, 116.22, 114.55, 37.50, 29.58, 28.75, 22.49, 22.41. HRMS (ESI) m/z calcd for C₂₅H₂₂N₆O [M+H]⁺423.1927, found 423.1926.

### N-((1-methyl-1H-imidazol-2-yl)methyl)-4-(8,9,10,11-tetrahydro-3H-pyrazolo[4,3-a]phenanthridin-7-yl)benzamide (Compound 7)

Following the same synthetic procedure as Compound 4, Compound 7 was obtained as a pale-yellow solid (68 mg, 31.2%). ¹H NMR (500 MHZ, (CD₃)₂SO) δ 8.56 (s, 1H), 8.37 (d, J=4.3 Hz, 3H), 8.16 (d, J=8.3 Hz, 3H), 7.04 (d, J=13.1 Hz, 1H), 6.78 (d, J=18.9 Hz, 1H), 4.56 (d, J=5.3 Hz, 2H), 3.68 (s, 3H), 3.58 (s, 2H) 2.77 (t, J=6.2 Hz, 3H), 1.88 (dp, J=127.7, 5.7 Hz, 6H), 1.23 (dd, J=14.7, 7.0 Hz, 2H). ¹³C NMR (126 MHZ, (CD₃)₂SO) δ 166.48, 156.10, 147.20, 144.21, 142.53, 139.90, 134.92, 133.82, 129.52, 129.41, 129.19, 127.72, 127.58, 126.87, 122.20, 119.24, 36.04, 32.94, 29.66, 28.80, 22.59, 22.52. HRMS (ESI) m/z calcd for C₂₆H₂₅N₆O [M+H]⁺437.2084, found 437.2081.

### N-((4H-1,2,4-triazol-3-yl)methyl)-4-(8,9,10,11-tetrahydro-3H-pyrazolo[4,3-a]phenanthridin-7-yl)benzamide (Compound 8)

Following the same synthetic procedure as Compound 4, Compound 8 was obtained as an off-white solid (79 mg, 37.4%). ¹H NMR (500 MHZ, (CD₃)₂SO) δ 8.58 (s, 1H), 8.43-8.06 (m, 2H), 8.01 (d, J=7.8 Hz, 2H), 7.84 (q, J=9.1 Hz, 2H), 7.65 (d, J=7.8 Hz, 2H), 4.61 (d, J=5.6 Hz, 2H), 3.34 (d, J=13.1 Hz, 2H), 2.78 (t, J=6.1 Hz, 2H), 1.89 (dp, J=125.7, 6.1 Hz, 4H). 13C NMR (126 MHZ, (CD₃)₂SO) δ 166.66, 161.54, 156.17, 148.63, 144.20, 143.89, 142.55, 133.95, 129.66, 129.41, 129.24, 127.56, 122.14, 119.78, 116.29, 36.76, 29.66, 28.81, 22.59, 22.53. HRMS (ESI) m/z calcd for C₂₄H₂₁N₇O [M+H]⁺424.1880, found 424.1877.

### N-((1H-imidazol-2-yl)methyl)-3-fluoro-4-(8,9,10,11-tetrahydro-3H-pyrazolo[4,3-a]phenanthridin-7-yl)benzamide (Compound 9)

Following the same synthetic procedure as Compound 4, Compound 9 was obtained as an off-white solid (109 mg, 49.5%). ¹H NMR (500 MHZ, (CD₃)₂SO) δ 8.59 (s, 1H), 7.93-7.78 (m, 4H), 7.57 (t, J=7.6 Hz, 1H), 7.16 (s, 2H), 4.65 (d, J=5.5 Hz, 2H), 3.35 (t, J=6.5 Hz, 2H), 2.62 (t, J=6.2 Hz, 2H), 2.04-1.74 (m, 4H). ¹³C NMR (126 MHZ, (CD₃)₂SO) δ 165.63, 161.85, 159.52 (1J=246.4 Hz), 151.59, 151.13, 145.34, 144.18, 143.97, 142.50, 136.45, 131.98, 131.87, 129.97, 129.47, 129.06, 124.10, 122.51, 121.17, 116.22, 115.16 (2J=20 Hz), 36.92, 29.47, 27.37, 22.56, 22.17. HRMS (ESI) m/z calcd for C₂₅H₂₁FN₆O [M+H]⁺441.1833, found 441.1830.

### N-((1H-tetrazol-5-yl)methyl)-4-(8,9,10,11-tetrahydro-3H-pyrazolo[4,3-a]phenanthridin-7-yl)benzamide (Compound 10)

To a solution of the alkyne (1 eq) in DMF (2 mL) and water (0.5 mL) was added TMS-azide (2 eq). Catalytic amount of copper sulfide pentahydrate and sodium ascorbate added. The reaction mixture was allowed to stir at 100° C. overnight. The reaction solvent was evaporated using a rotary evaporator, the residue was diluted with ethyl acetate, washed with water, and dried over sodium sulfate. It was then purified via column chromatography with 90% ethyl acetate and 10% hexanes to afford an off-white solid (36 mg, 34%).

¹H NMR (500 MHZ, (CD₃)₂SO) δ 9.43 (dt, J=33.1, 5.5 Hz, 1H), 8.79 (d, J=3.9 Hz, 1H), 8.33-8.08 (m, 4H), 7.83 (dd, J=8.1, 3.9 Hz, 2H), 4.82 (d, J=5.6 Hz, 2H), 3.50 (t, J=6.2 Hz, 2H), 2.78 (t, J=6.2 Hz, 2H), 2.15-1.98 (m, 2H), 1.82 (ddt, J=11.9, 9.4, 4.1 Hz, 2H). ¹³C NMR (126 MHZ, (CD₃)₂SO) δ 166.55, 155.31, 151.46, 140.50, 137.36, 135.41, 131.33, 130.29, 130.13, 128.06, 128.01, 123.41, 122.54, 120.16, 117.93, 115.22, 33.87, 30.66, 28.29, 28.13, 21.92, 21.80. HRMS (ESI) m/z calcd for C₂₃H₂₀N₈O [M+H]⁺425.1833, found 425.1831.

### N-((1H-imidazol-2-yl)methyl)-2-fluoro-4-(8,9,10,11-tetrahydro-3H-pyrazolo[4,3-a]phenanthridin-7-yl)benzamide (Compound 11)

Following the same synthetic procedure as Compound 4, Compound 11 was obtained as an off-white solid (87 mg, 39.5%). ¹H NMR (500 MHZ, (CD₃)₂SO) δ 8.82 (q, J=4.9 Hz, 1H), 8.55 (s, 1H), 7.83 (dq, J=22.1, 9.0, 7.4 Hz, 3H), 7.50 (t, J=10.4 Hz, 2H), 6.97 (s, 2H), 4.55 (d, J=5.6 Hz, 2H), 3.25 (t, J=6.7 Hz, 2H), 2.76 (t, J=6.1 Hz, 2H), 1.96 (p, J=6.5, 6.1 Hz, 2H), 1.72 (p, J=5.7 Hz, 2H). ¹³C NMR (126 MHz, (CD₃)₂SO) δ 164.00, 161.89, 160.52 (1J=249.4 Hz), 154.65, 149.62, 145.80, 145.73, 145.06, 143.72, 142.71, 138.50, 130.58, 129.58, 129.18, 125.69, 122.93, 122.82, 122.25, 117.29 (2J=23 Hz), 116.18, 37.66, 29.62, 28.70, 22.48, 22.40. HRMS (ESI) m/z calcd for C₂₅H₂₁FN₆O [M+H]⁺441.1873, found 441.1837.

### N-((1H-pyrrol-3-yl)methyl)-4-(8,9,10,11-tetrahydro-3H-pyrazolo[4,3-a]phenanthridin-7-yl)benzamide (Compound 12)

Following the same synthetic procedure as Compound 4, Compound 12 was obtained as a light brown solid (77 mg, 36.5%). ¹H NMR (500 MHZ, (CD₃)₂SO) δ 8.47 (s, 1H), 8.01 (d, J=8.0 Hz, 2H), 7.79 (q, J=9.1 Hz, 2H), 7.60 (d, J=7.9 Hz, 2H), 6.74 (t, J=2.1 Hz, 1H), 6.67 (q, J=2.4 Hz, 1H), 6.08 (q, J=2.3 Hz, 1H), 4.41 (d, J=5.6 Hz, 2H), 3.53 (p, J=6.6 Hz, 2H), 3.17 (t, J=6.4 Hz, 2H), 1.89 (q, J=7.6, 6.3 Hz, 2H), 1.63 (p, J=6.1, 5.7 Hz, 2H). ¹³C NMR (126 MHZ, (CD₃)₂SO) & 166.30, 165.17, 162.82, 156.02, 143.84, 143.75, 142.45, 134.41, 129.52, 129.36, 129.11, 127.37, 127.01, 121.99, 120.98, 118.03, 116.28, 116.22, 107.90, 54.09, 36.02, 22.38, 22.33, 12.65. HRMS (ESI) m/z calcd for C₂₆H₂₃N₅O [M+H]⁺422.1981, found 422.1971.

### 4-(8,9,10,11-tetrahydro-3HI-pyrazolo[4,3-a]phenanthridin-7-yl)-N-(thiazol-2-ylmethyl)benzamide (Compound 13)

Following the same synthetic procedure as Compound 4, Compound 13 was obtained as an off-white solid (65 mg, 29.8%). ¹H NMR (500 MHZ, (CD₃)₂SO) δ 9.40 (t, J=6.0 Hz, 1H), 8.57 (s, 1H), 8.07-7.98 (m, 2H), 7.90-7.78 (m, 2H), 7.73 (d, J=3.3 Hz, 1H), 7.72-7.65 (m, 2H), 7.61 (d, J=3.3 Hz, 1H), 4.80 (d, J=5.9 Hz, 2H), 3.33 (s, 2H), 2.78 (t, J=6.1 Hz, 2H), 2.05-1.96 (m, 2H), 1.84-1.71 (m, 2H). ¹³C NMR (126 MHZ, (CD₃)₂SO) δ 169.89, 166.81, 156.08, 144.47, 142.64, 142.58, 136.40, 133.52, 129.66, 129.63, 129.29, 127.52, 127.07, 122.13, 120.49, 114.52, 41.53, 29.67, 28.83, 22.58, 22.51. HRMS (ESI) m/z calcd for C₂₅H₂₁N₅OS [M+H]⁺440.1545, found 440.1547.

### N-((4-methyl-1H-imidazol-2-yl)methyl)-4-(8,9,10,11-tetrahydro-3H-pyrazolo[4,3-a]phenanthridin-7-yl)benzamide (Compound 14, otherwise known as Compound HSK205)

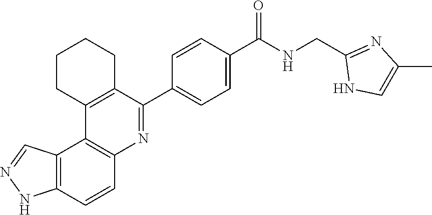 [SMILES: Cc1c[nH]c(CNC(=O)c2ccc(-c3nc4ccc5[nH]ncc5c4c4c3CCCC4)cc2)n1]

Following the same synthetic procedure as Compound 4, Compound 14 was obtained as an off-white solid (89 mg, 40.7%). ¹H NMR (500 MHZ, (CD₃)₂SO) δ 9.01 (t, J=5.7 Hz, 1H), 8.57 (s, 1H), 8.05-7.99 (m, 2H), 7.89-7.80 (m, 2H), 7.68-7.63 (m, 2H), 6.63-6.58 (m, 1H), 4.46 (d, J=5.7 Hz, 2H), 2.77 (t, J=6.1 Hz, 2H), 2.09 (d, J=1.0 Hz, 3H), 2.07 (s, 1H), 2.01 (dd, J=7.8, 3.9 Hz, 2H), 1.97 (s, 1H), 1.75 (dd, J=7.6, 4.2 Hz, 2H). ¹³C NMR (126 MHZ, (CD₃)₂SO) δ 166.47, 156.23, 149.06, 144.60, 144.08, 143.78, 142.59, 138.74, 136.44, 133.83, 132.46, 129.67, 129.44, 127.61, 122.02, 116.46, 114.56, 105.50, 60.23, 37.63, 29.71, 28.87, 22.58, 14.56. HRMS (ESI) m/z calcd for C₂₆H₂₄N₆O [M+H]⁺437.2079, found 437.2083.

### N-(2-(1H-imidazol-2-yl)ethyl)-4-(8,9,10,11-tetrahydro-3H-pyrazolo[4,3-a]phenanthridin-7-yl)benzamide (Compound 15)

Following the same synthetic procedure as Compound 4, Compound 15 was obtained as an off-white solid (73 mg, 33.4%). ¹H NMR (500 MHZ, (CD₃)₂SO) δ 8.63 (t, J=5.6 Hz, 1H), 8.57 (s, 1H), 7.93-7.89 (m, 2H), 7.87-7.77 (m, 2H), 7.64 (d, J=8.3 Hz, 2H), 7.16 (d, J=21.5 Hz, 1H), 6.90 (d, J=10.4 Hz, 1H), 4.21 (t, J=6.1 Hz, 2H), 3.63 (q, J=5.9 Hz, 2H), 3.42 (q, J=6.0 Hz, 2H), 2.76 (t, J=6.1 Hz, 2H), 1.99 (ddd, J=15.1, 7.5, 4.5 Hz, 2H), 1.73 (ddd, J=9.0, 7.2, 4.2 Hz, 2H). ¹³C NMR (126 MHZ, (CD₃)₂SO) δ 166.94, 161.91, 156.11, 144.18, 143.82, 142.54, 137.83, 134.00, 129.65, 129.49, 129.24, 128.72, 128.67, 127.35, 122.11, 120.11, 119.92, 116.35, 45.82, 38.81, 29.65, 28.83, 22.57, 22.49. HRMS (ESI) m/z calcd for C₂₆H₂₄N₆O [M+H]⁺437.2084, found 437.2070.

### N-((4-methyl-1H-imidazol-2-yl)methyl)-4-(1-methyl-8,9,10,11-tetrahydro-3H-pyrazolo[4,3-a]phenanthridin-7-yl)benzamide

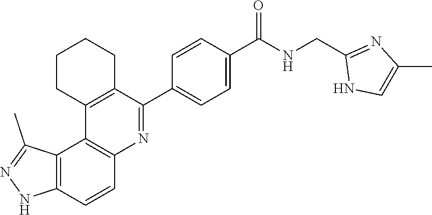 [SMILES: Cc1c[nH]c(CNC(=O)c2ccc(-c3nc4ccc5[nH]nc(C)c5c4c4c3CCCC4)cc2)n1]

Following the same synthetic procedure as Compound HSK205, the compound was obtained as a pale-yellow solid (119 mg, 52.9%). ¹H NMR (500 MHZ, (CD₃)₂SO) δ 8.01 (d, J=7.9 Hz, 2H), 7.79-7.65 (m, 2H), 7.61 (d, J=8.0 Hz, 2H), 6.65 (s, 1H), 4.48 (d, J=4.9 Hz, 2H), 3.40 (s, 2H), 2.85 (d, J=5.8 Hz, 3H), 2.75 (s, 2H), 2.10 (s, 3H), 1.77 (s, 4H). ¹³C NMR (201 MHz, (CD₃)₂SO) δ 165.37, 159.24, 155.88, 151.77, 145.26, 143.68, 142.67, 138.74, 136.48, 131.87, 129.78, 129.65, 129.36, 128.38, 122.14, 116.45, 114.66, 114.39, 41.00, 39.19, 31.16, 29.69, 28.85, 22.57, 22.47. HRMS (ESI) m/z calcd for C₂₇H₂₆N₆O [M+H]⁺451.2241, found 451.2230.

### 3-fluoro-4-(9-hydroxy-1-methyl-8,9,10,11-tetrahydro-3H-pyrazolo[4,3-a]phenanthridin-7-yl)-N-(1-(4-methyl-1H-imidazol-2-yl)ethyl)benzamide

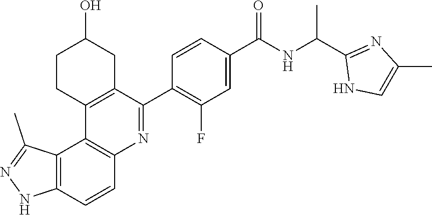 [SMILES: Cc1c[nH]c(C(C)NC(=O)c2ccc(-c3nc4ccc5[nH]nc(C)c5c4c4c3CC(O)CC4)c(F)c2)n1]

Following the same synthetic procedure as Compound HSK205, the compound was obtained as an off-white solid (98 mg, 39.3%). ¹H NMR (500 MHZ, (CD₃)₂SO) δ 8.43 (d, J=7.0 Hz, 2H), 7.95-7.87 (m, 2H), 7.81 (dd, J=8.9, 2.5 Hz, 1H), 5.31 (dq, J=7.0, 4.8 Hz, 1H), 4.09-4.00 (m, 2H), 3.90 (d, J=4.9 Hz, 2H), 3.48 (dd, J=17.5, 7.1 Hz, 1H), 3.38 (ddd, J=15.6, 9.1, 6.4 Hz, 1H), 3.30-3.18 (m, 2H), 2.28 (s, 3H), 2.05-1.97 (m, 3H), 1.86-1.75 (m, 3H). ¹³C NMR (201 MHZ, (CD₃)₂SO) δ 165.89, 160.87, 159.26, 150.77, 147.34, 144.09, 141.82, 137.27, 131.54, 130.91, 129.13, 128.54, 128.31, 128.19, 127.76, 126.99, 126.58, 117.67, 117.01, 115.13, 56.76, 51.42, 36.68, 30.21, 26.56, 21.85, 19.44, 14.02.

### 3-fluoro-4-(9-hydroxy-8,9,10,11-tetrahydro-3H-pyrazolo[4,3-a]phenanthridin-7-yl)-N-((4-methyl-1H-imidazol-2-yl)methyl)benzamide

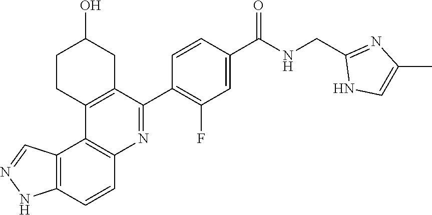 [SMILES: Cc1c[nH]c(CNC(=O)c2ccc(-c3nc4ccc5[nH]ncc5c4c4c3CC(O)CC4)c(F)c2)n1]

Following the same synthetic procedure as Compound HSK205, the compound was obtained as a yellow solid (87 mg, 37.1%). ¹H NMR (500 MHZ, (CD₃)₂SO) δ 8.74 (s, 1H), 7.98 (d, J=9.2 Hz, 2H), 7.92-7.79 (m, 3H), 7.12-7.07 (m, 1H), 4.49 (s, 2H), 4.01 (ttd, J=7.9, 6.0, 4.2 Hz, 1H), 3.90 (d, J=6.1 Hz, 2H), 3.39-3.25 (m, 2H), 3.18 (ddd, J=15.6, 8.8, 6.6 Hz, 2H), 1.95-1.79 (m, 3H). ¹³C NMR (201 MHz, (CD₃)₂SO) δ 165.69, 160.34, 158.28, 149.06, 147.30, 144.08, 142.58, 138.14, 133.23, 131.46, 129.87, 129.03, 127.91, 127.72, 127.55, 126.98, 126.74, 119.20, 115.16, 109.66, 60.19, 39.52, 29.71, 28.94, 23.67, 13.96. HRMS (ESI) m/z calcd for C₂₆H₂₃FN₆O₂ [M+H]⁺471.1967, found 471.1963.

### 3-fluoro-N-((4-methyl-1H-imidazol-2-yl)methyl)-4-(8,9,10,11-tetrahydro-3H-pyrazolo[4,3-a]phenanthridin-7-yl)benzamide

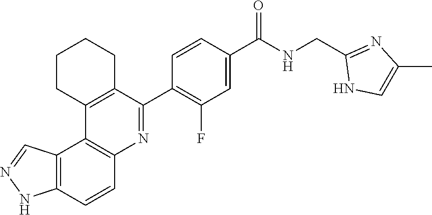 [SMILES: Cc1c[nH]c(CNC(=O)c2ccc(-c3nc4ccc5[nH]ncc5c4c4c3CCCC4)c(F)c2)n1]

Following the same synthetic procedure as Compound HSK205, the compound was obtained as an off-white solid (69 mg, 30.4%). ¹H NMR (500 MHZ, (CD₃)₂SO) δ 8.72 (s, 1H), 7.94 (d, J=8.9 Hz, 1H), 7.90-7.81 (m, 2H), 7.09-7.01 (m, 2H), 4.56 (s, 2H), 3.37-3.28 (m, 4H), 1.93-1.85 (m, 4H), 1.83-1.73 (m, 3H). ¹³C NMR (201 MHz, (CD₃)₂SO) δ 166.57, 166.51, 161.57, 159.58, 153.97, 153.91, 150.38, 144.34, 142.00, 141.52, 135.30, 135.27, 135.21, 132.37, 132.31, 131.28, 129.46, 129.35, 126.23, 125.52, 125.49, 124.22, 124.19, 120.45, 116.80, 115.62, 114.27, 114.09, 113.63, 36.61, 28.38, 27.08, 22.70, 21.62, 14.20. HRMS (ESI) m/z calcd for C₂₆H₂₃FN₆O [M+H]⁺455.1927, found 455.1936.

### N-((4-methyl-1H-imidazol-2-yl)methyl)-2-(4-(8,9,10,11-tetrahydro-3H-pyrazolo[4,3-a]phenanthridin-7-yl)phenyl) acetamide

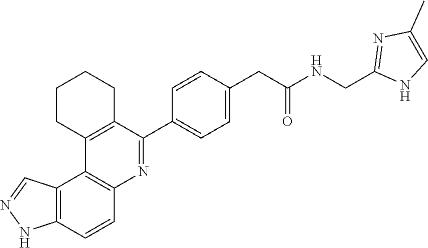 [SMILES: Cc1c[nH]c(CNC(=O)Cc2ccc(-c3nc4ccc5[nH]ncc5c4c4c3CCCC4)cc2)n1]

Following the same synthetic procedure as Compound HSK205, the compound was obtained as a pale-yellow solid (70 mg, 30.6%). ¹H NMR (500 MHZ, (CD₃)₂SO) δ 8.68 (s, 1H), 7.97 (d, J=9.2 Hz, 2H), 7.83-7.74 (m, 4H), 6.45 (dt, J=8.4, 0.9 Hz, 1H), 4.40 (s, 2H), 4.21 (s, 2H), 3.60 (t, J=0.9 Hz, 2H), 3.31-3.21 (m, 4H), 2.04-1.91 (m, 2H), 1.86 (s, 3H). ¹³C NMR (201 MHz, (CD₃)₂SO) δ 169.09, 153.62, 148.76, 143.62, 141.18, 141.07, 136.44, 135.77, 135.50, 132.39, 128.85, 128.70, 126.44, 126.02, 119.66, 116.66, 116.39, 113.46, 41.38, 36.96, 27.86, 27.39, 22.62, 21.75, 14.26. HRMS (ESI) m/z calcd for C₂₇H₂₆N₆O [M+H]⁺451.2253, found 451.2251.

### 4-(1-methoxy-8,9,10,11-tetrahydro-3H-pyrazolo[4,3-a]phenanthridin-7-yl)-N-((4-methyl-1H-imidazol-2-yl)methyl)benzamide

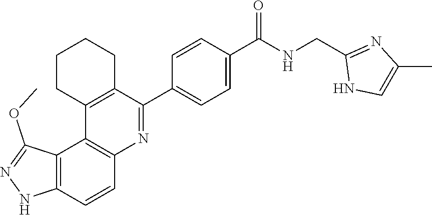 [SMILES: COc1n[nH]c2ccc3nc(-c4ccc(C(=O)NCc5nc(C)c[nH]5)cc4)c4c(c3c12)CCCC4]

Following the same synthetic procedure as Compound HSK205, the compound was obtained as a white solid (109 mg, 46.7%). ¹H NMR (500 MHZ, (CD₃)₂SO) δ 8.64 (s, 1H), 8.04-7.97 (m, 2H), 7.88-7.78 (m, 2H), 7.69-7.61 (m, 2H), 6.61-6.56 (m, 1H), 4.44 (d, J=5.6 Hz, 2H), 4.07 (s, 3H), 2.79 (t, J=6.2 Hz, 2H), 2.11 (d, J=1.0 Hz, 3H), 2.07 (s, 1H), 2.02 (dd, J=7.6, 3.9 Hz, 2H), 1.94 (s, 1H), 1.71 (dd, J=7.2, 4.0 Hz, 2H).

### N-(1-(4-methyl-1H-imidazol-2-yl)ethyl)-4-(8,9,10,11-tetrahydro-3H-pyrazolo[4,3-a]phenanthridin-7-yl)benzamide

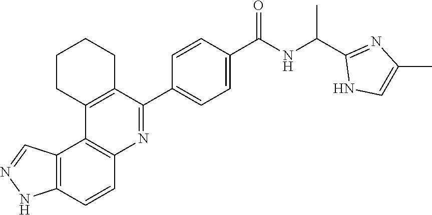 [SMILES: Cc1c[nH]c(C(C)NC(=O)c2ccc(-c3nc4ccc5[nH]ncc5c4c4c3CCCC4)cc2)n1]

Following the same synthetic procedure as Compound HSK205, the compound was obtained as a pale-yellow solid (101 mg, 44.8%). ¹H NMR (500 MHZ, (CD₃)₂SO) δ 8.75 (s, 1H), 7.98-7.86 (m, 4H), 7.81 (d, J=9.1 Hz, 2H), 6.82 (dd, J=6.1, 0.9 Hz, 1H), 5.04 (dq, J=7.0, 4.8 Hz, 1H), 3.31-3.21 (m, 4H), 1.93-1.84 (m, 4H), 1.82 (d, J=4.8 Hz, 3H), 1.81-1.74 (m, 3H). ¹³C NMR (201 MHz, (CD₃)₂SO) δ 165.83, 153.97, 151.95, 143.62, 141.16, 141.17, 139.49, 136.75, 135.11, 132.38, 128.52, 126.64, 126.48, 126.02, 119.66, 117.14, 116.66, 113.46, 46.03, 27.86, 27.39, 22.62, 21.75, 19.16, 13.27.

### N-(1-(4-methyl-1H-imidazol-2-yl)ethyl)-4-(1-methyl-8,9,10,11-tetrahydro-3H-pyrazolo[4,3-a]phenanthridin-7-yl)benzamide

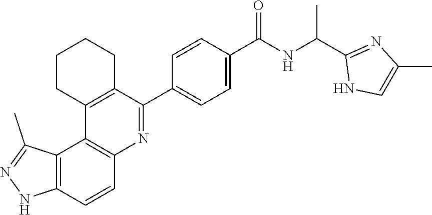 [SMILES: Cc1c[nH]c(C(C)NC(=O)c2ccc(-c3nc4ccc5[nH]nc(C)c5c4c4c3CCCC4)cc2)n1]

Following the same synthetic procedure as Compound HSK205, the compound was obtained as a light orange solid (23 mg, 9.9%). ¹H NMR (500 MHZ, (CD₃)₂SO) δ 7.96-7.87 (m, 4H), 7.80 (d, J=9.3 Hz, 2H), 6.85 (dd, J=6.1, 0.6 Hz, 1H), 5.01 (dq, J=6.6, 4.2 Hz, 1H), 3.34 (d, J=13.5 Hz, 2H), 3.27-3.21 (m, 4H), 2.28-2.22 (m, 3H), 2.09 (s, 3H), 1.93-1.81 (m, 2H), 1.85-1.77 (m, 3H). ¹³C NMR (201 MHz, (CD₃)₂SO) δ 165.79, 153.88, 151.87, 144.21, 143.70, 141.64, 140.87, 139.49, 136.75, 135.21, 128.52, 126.81, 126.61, 125.46, 120.93, 117.33, 117.14, 112.10, 46.11, 27.67, 27.53, 22.58, 21.73, 19.21, 18.68, 13.61.

### N-(2-(4-methyl-1H-imidazol-2-yl) propan-2-yl)-4-(8,9,10,11-tetrahydro-3H-pyrazolo[4,3-a]phenanthridin-7-yl)benzamide

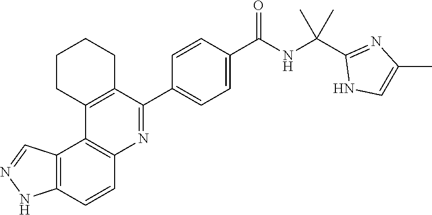 [SMILES: Cc1c[nH]c(C(C)(C)NC(=O)c2ccc(-c3nc4ccc5[nH]ncc5c4c4c3CCCC4)cc2)n1]

Following the same synthetic procedure as Compound HSK205, the compound was obtained as an off-white solid (84 mg, 36.2%). ¹H NMR (500 MHZ, (CD₃)₂SO) δ 8.61 (s, 1H), 7.99-7.92 (m, 2H), 7.92-7.85 (m, 2H), 7.81 (d, J=8.8 Hz, 1H), 6.46-6.41 (m, 1H), 3.31 (s, 3H) 3.12 (s, 3H), 1.93-1.83 (m, 4H), 1.83-1.73 (m, 4H), 1.79 (s, 3H). ¹³C NMR (201 MHz, (CD₃)₂SO) δ 166.23, 153.96, 151.23, 143.62, 141.18, 141.07, 139.41, 136.56, 136.13, 132.39, 128.52, 127.37, 126.48, 126.02, 119.66, 116.93, 116.66, 113.46, 50.07, 27.86, 27.52, 27.39, 22.62, 21.75, 12.60.

### N-(2-(4-methyl-1H-imidazol-2-yl) propan-2-yl)-4-(1-methyl-8,9,10,11-tetrahydro-3H-pyrazolo[4,3-a]phenanthridin-7-yl)benzamide

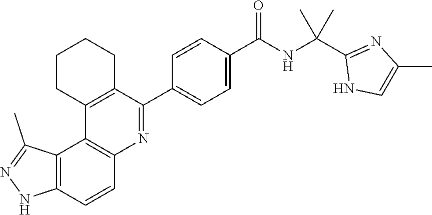 [SMILES: Cc1c[nH]c(C(C)(C)NC(=O)c2ccc(-c3nc4ccc5[nH]nc(C)c5c4c4c3CCCC4)cc2)n1]

Following the same synthetic procedure as Compound HSK205, the compound was obtained as a white solid (109 mg, 45.6%). ¹H NMR (500 MHZ, (CD₃)₂SO) δ 7.98-7.88 (m, 2H), 7.91-7.85 (m, 2H), 7.46-7.41 (m, 2H), 3.35 (s, 3H), 3.21 (s, 3H), 2.30 (s, 3H), 1.92-1.84 (m, 4H), 1.80-1.71 (m, 4H), 1.72 (s, 3H). ¹³C NMR (201 MHz, (CD₃)₂SO) & 166.19, 153.98, 151.27, 144.21, 143.70, 141.64, 140.87, 139.41, 136.56, 136.13, 128.52, 127.37, 126.81, 125.46, 120.93, 117.33, 116.93, 112.10, 50.07, 27.67, 27.53, 27.52, 22.62, 21.75, 18.68, 10.60. HRMS (ESI) m/z calcd for C₂₉H₃₀N₆O [M+H]⁺479.2411, found 479.2422.

### N-((4-ethyl-1H-imidazol-2-yl)methyl)-4-(1-methyl-8,9,10,11-tetrahydro-3H-pyrazolo[4,3-a]phenanthridin-7-yl)benzamide

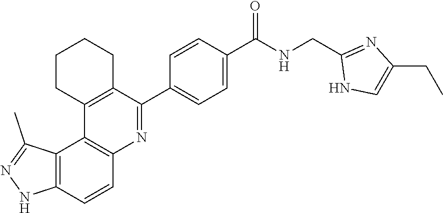 [SMILES: CCc1c[nH]c(CNC(=O)c2ccc(-c3nc4ccc5[nH]nc(C)c5c4c4c3CCCC4)cc2)n1]

Following the same synthetic procedure as Compound HSK205, the compound was obtained as a yellow solid (43 mg, 18.5%). ¹H NMR (500 MHZ, (CD₃)₂SO) δ 8.07-7.96 (m, 4H), 7.80 (d, J=9.2 Hz, 2H), 6.23 (d, J=4.9 Hz, 1H), 4.48 (s, 2H), 3.39 (d, J=13.1 Hz, 2H), 3.29-3.20 (m, 4H), 2.70 (q, J=7.1 Hz, 2H), 1.93-1.82 (m, 2H), 1.75 (s, 3H), 1.28 (t, J=6.6 Hz, 3H). ¹³C NMR (201 MHz, (CD₃)₂SO) δ 165.86, 153.98, 150.50, 144.23, 143.72, 141.64, 140.89, 139.56, 139.48, 134.48, 128.52, 126.95, 126.80, 125.44, 120.93, 117.36, 116.07, 112.10, 36.72, 27.64, 27.56, 22.59, 21.78, 20.65, 18.79, 13.33. HRMS (ESI) m/z calcd for C₂₈H₂₈N₆O [M+H]⁺465.2347, found 465.2340.

### 4-(8,9,10,11-tetrahydro-3H-pyrazolo[4,3-a]phenanthridin-7-yl)-N-((4-(trifluoromethyl)-1H-imidazol-2-yl)methyl)benzamide

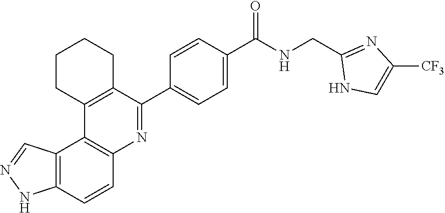 [SMILES: O=C(NCc1nc(C(F)(F)F)c[nH]1)c1ccc(-c2nc3ccc4[nH]ncc4c3c3c2CCCC3)cc1]

Following the same synthetic procedure as Compound HSK205, the compound was obtained as a yellow-orange solid (217 mg, 88.4%). ¹H NMR (500 MHZ, (CD₃)₂SO) δ 8.66 (s, 1H), 7.99-7.87 (m, 4H), 7.81 (d, J=9.1 Hz, 2H), 6.72 (dq, J=4.1, 2.3 Hz, 1H), 4.57 (s, 2H), 3.32-3.21 (m, 4H), 1.92-1.83 (m, 4H). ¹³C NMR (201 MHZ, (CD₃)₂SO) δ 165.89, 153.96, 150.15, 150.11, 150.08, 150.04, 143.62, 141.18, 141.07, 139.56, 134.48, 132.39, 129.61, 129.31, 129.01, 128.71, 128.52, 126.95, 126.48, 126.02, 124.37, 122.19, 120.02, 119.66, 117.85, 116.66, 116.35, 116.32, 116.29, 116.26, 113.46, 36.87, 27.86, 27.39, 22.62, 21.75. HRMS (ESI) m/z calcd for C₂₆H₂₁F₃N₆O [M+H]⁺491.1781, found 491.1785.

### 4-(1-methyl-8,9,10,11-tetrahydro-3H-pyrazolo[4,3-a]phenanthridin-7-yl)-N-((4-(trifluoromethyl)-1H-imidazol-2-yl)methyl)benzamide

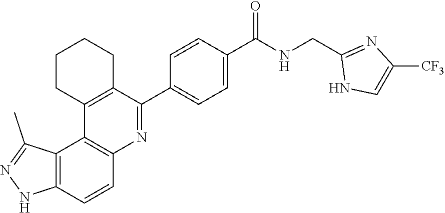 [SMILES: Cc1n[nH]c2ccc3nc(-c4ccc(C(=O)NCc5nc(C(F)(F)F)c[nH]5)cc4)c4c(c3c12)CCCC4]

Following the same synthetic procedure as Compound HSK205, the compound was obtained as an off-white solid (197 mg, 78.1%). ¹H NMR (500 MHZ, (CD₃)₂SO) δ 7.97-7.87 (m, 4H), 7.80 (d, J=8.8 Hz, 2H), 6.68 (dq, J=4.0, 1.8 Hz, 1H), 4.49 (s, 2H), 3.18 (d, J=10.9 Hz, 2H), 3.27-3.20 (m, 4H), 1.85 (s, 3H), 1.83-1.76 (m, 2H). ¹³C NMR (201 MHz, (CD₃)₂SO) δ 165.91, 153.98, 150.16, 150.13, 150.09, 150.03, 144.21, 143.70, 141.64, 140.87, 139.56, 134.48, 129.62, 129.37, 129.05, 128.70, 128.54, 126.99, 126.81, 125.46, 124.40, 122.21, 120.93, 120.04, 117.85, 117.33, 116.37, 116.35, 116.29, 116.27, 112.10, 36.79, 27.67, 27.53, 22.59, 21.67, 18.68. HRMS (ESI) m/z calcd for C₂₇H₂₃F₃N₆O [M+H]⁺505.1925, found 505.1918.

### N-methyl-N-((4-methyl-1H-imidazol-2-yl)methyl)-4-(8,9,10,11-tetrahydro-3H-pyrazolo[4,3-a]phenanthridin-7-yl)benzamide

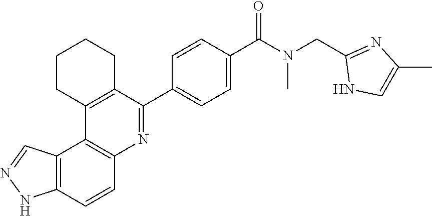 [SMILES: Cc1c[nH]c(CN(C)C(=O)c2ccc(-c3nc4ccc5[nH]ncc5c4c4c3CCCC4)cc2)n1]

¹H NMR (500 MHZ, (CD₃)₂SO) δ 9.01 (t, J=5.7 Hz, 1H), 8.55 (s, 1H), 8.07-7.99 (m, 2H), 7.88-7.80 (m, 2H), 7.68-7.63 (m, 2H), 6.63-6.58 (m, 1H), 4.42 (d, J=5.7 Hz, 2H), 2.76 (t, J=6.3 Hz, 2H), 2.61 (s, 3H), 2.09 (d, J=1.0 Hz, 3H), 2.05 (s, 1H), 2.01 (dd, J=7.8, 3.9 Hz, 2H), 1.94 (s, 1H), 1.77 (dd, J=7.5, 4.1 Hz, 2H).

### N-((4-methyl-1H-imidazol-2-yl)methyl)-4-(3H-pyrazolo[4,3-f]quinolin-7-yl)benzamide

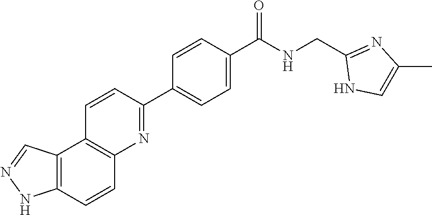 [SMILES: Cc1c[nH]c(CNC(=O)c2ccc(-c3ccc4c(ccc5[nH]ncc54)n3)cc2)n1]

¹H NMR (500 MHZ, (CD₃)₂SO) δ 8.99 (t, J=5.5 Hz, 1H), 8.52 (s, 1H), 8.46-8.39 (m, 2H), 8.11-8.01 (m, 2H), 7.89-7.81 (m, 2H), 7.69-7.62 (m, 2H), 6.61-6.55 (m, 1H), 4.44 (d, J=5.6 Hz, 2H), 2.08 (d, J=1.1 Hz, 3H).

### N-((4-methyl-1H-imidazol-2-yl)methyl)-4-(3′,8′,10′,11′-tetrahydrospiro[oxetane-3,9′-pyrazolo[4,3-a]phenanthridin]-7′-yl)benzamide

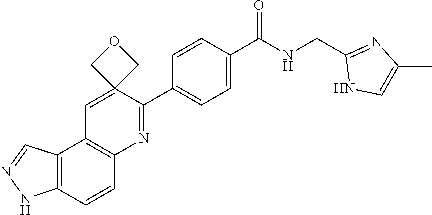

¹H NMR (500 MHZ, (CD₃)₂SO) δ 9.00 (t, J=5.6 Hz, 1H), 8.55 (s, 1H), 8.08-7.96 (m, 2H), 7.89-7.80 (m, 2H), 7.66-7.61 (m, 2H), 6.66-6.59 (m, 1H), 4.43 (d, J=5.6 Hz, 2H), 3.89 (d, J=8.1 Hz, 2H), 3.65 (d, J=8.2 Hz, 2H), 2.77 (t, J=6.1 Hz, 2H), 2.61 (s, 3H), 2.09 (d, J=1.1 Hz, 3H), 2.04 (s, 1H), 2.01 (dd, J=7.7, 3.6 Hz, 2H), 1.91 (s, 1H), 1.72 (dd, J=7.7, 4.2 Hz, 2H).

### N-((4-methyl-1H-imidazol-2-yl)methyl)-4-(2,3,3′,5,6,8′,10′,11′-octahydrospiro[pyran-4,9′-pyrazolo[4,3-a]phenanthridin]-7′-yl)benzamide

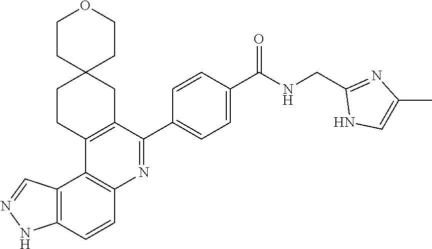 [SMILES: Cc1c[nH]c(CNC(=O)c2ccc(-c3nc4ccc5[nH]ncc5c4c4c3CC3(CCOCC3)CC4)cc2)n1]

¹H NMR (500 MHZ, (CD₃)₂SO) δ 8.59 (s, 1H), 8.12-7.99 (m, 2H), 7.86-7.79 (m, 2H), 7.66-7.60 (m, 2H), 6.69-6.58 (m, 1H), 4.44 (d, J=5.5 Hz, 2H), 3.71 (dt, J=10.8, 2.8 Hz, 2H), 3.62 (dt, J=10.8, 6.5 Hz, 2H), 2.77 (t, J=6.1 Hz, 2H), 2.61 (s, 3H), 2.09 (d, J=1.1 Hz, 3H), 2.04 (s, 1H), 2.01 (dd, J=7.7, 3.6 Hz, 2H), 1.97-1.88 (m, 5H), 1.72 (dd, J=7.7, 4.2 Hz, 2H).

1. A compound having a structure of formula I: 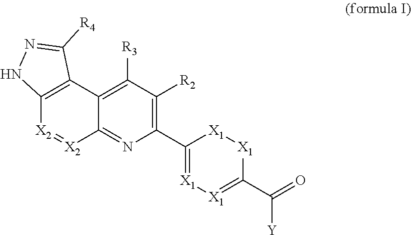
or a pharmaceutically acceptable salt thereof, wherein:
X₁ is each independently N, CH, or a C-halogen;
X₂ is each independently N, CH, a C-halogen, C—CHₙ₁Xₘ where n1+m=3 and X is a halogen, C—OCF₃, C—OCHF₂, C—OMe, C—Me, C—Et, C-tBu, C-iPr, C—N-alkyl, C—N-heteroalkyl, C—O-alkyl, or C—O-heteroalkyl;
R₂ and R₃are each independently an alkyl, cycloalkyl, heteroalkyl, or heterocycloalkyl;
R₄ is an H, alkyl, cycloalkyl, heteroalkyl, or heterocycloalkyl; and
Y is 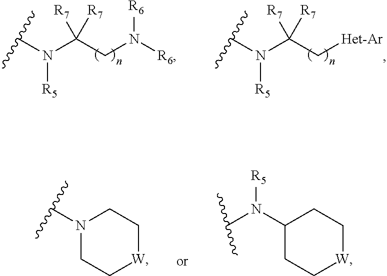
wherein:
n is 1 to 5,
R₅ and R₆ are each independently H, an alkyl, or a heteroalkyl, and R₆ can optionally form a cyclic structure,
R₇ is each independently H or an alkyl,
Het-Ar is a heteroaromatic ring, and
W is NH, NMe, NEt, NCH₂CH₂OH, NCH₂CH₂OMe, O, SO, or SO₂,
with the proviso that Y is not NH₂.

2. The compound of claim 1, wherein at least one X₁ and/or at least one X₂ is CF, CHF₂, CF₃, or CHF₃.

3. The compound of claim 1 having a structure of formula IA: 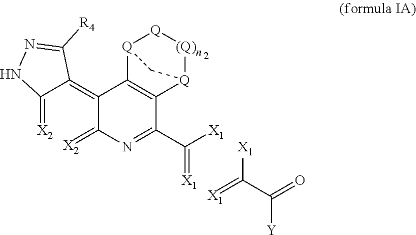
or a pharmaceutically acceptable salt thereof, wherein:
n2 is 1, 2 or 3;
Q is each independently O, NH, substituted N, CH₂, or substituted C, S, SO, or SO₂; and
each Q, taken together, form a 6-, 7-, or 8-membered ring or bicyclic ring.

4. The compound of claim 1, wherein one or more of R₅ and/or R₆ is Me and/or R₇ is each independently H or Me.

5. (canceled)

6. The compound of claim 1, wherein Y is 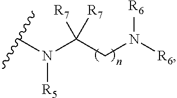
and each R₆, taken together, form a morpholine, piperdine, piperazine, or pyrrolidine; or
Y is 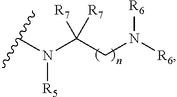
and each R₇, taken together, forms a cyclic structure; or
Y is 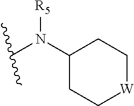
and R₅ is Me.

7. (canceled)

8. The compound of claim 6, wherein the cyclic structure formed by each R₇ comprises a cyclopropyl, a cyclobutyl, a cyclopentyl, or a cyclohexyl.

9-10. (canceled)

11. The compound of claim 1, wherein Y is 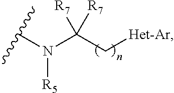
and Het-Ar comprises an imidazole, an oxazole, a pyrazole, a triazole, an isoxazole, an isothiazole, a tetrazole, an oxadiazole, a thiadiazole, a pyrimidine, or a triazine.

12. (canceled)

13. The compound of claim 3 having a structure of formula IB: 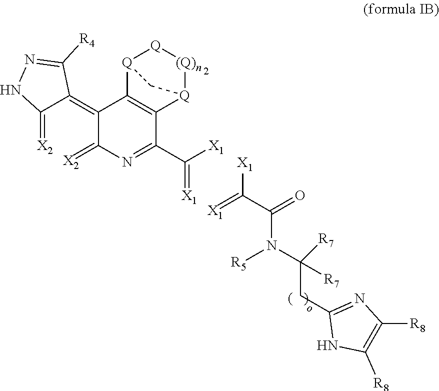
or a pharmaceutically acceptable salt thereof, wherein:
o is 0-2; and
each R₅ is independently H, an alkyl, or a heteroalkyl.

14. The compound of claim 13, wherein each R₇, taken together, forms a cyclic structure.

15. (canceled)

16. The compound of claim 13, wherein at least one Ra is Me, Et or CF₃.

17. The compound of claim 1 having a structure of formula II: 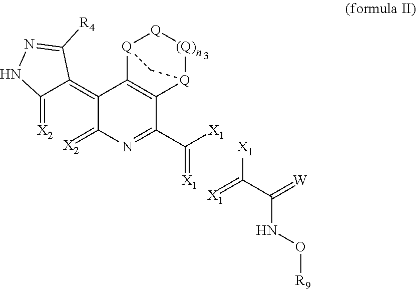
or a pharmaceutically acceptable salt thereof, wherein:
X₁ is each independently N, CH, or a C-halogen;
X₂ is each independently N, CH, a C-halogen, C—CHₙ₁Xₘ where n1+m=3 and X is a halogen, C—OCF₃, C—OCHF₂, C—OMe, C—Me, C—Et, C-tBu, C-iPr, C—N-alkyl, C—N-heteroalkyl, C—O-alkyl, or C—O-heteroalkyl;
Q is each independently O, NH, substituted N, CH₂, or substituted C, S, SO, or SO₂, and each Q, when taken together, form a 6-, 7-, or 8-membered ring or bicyclic ring;
n3 is 1, 2, or 3;
R₄ is an H, alkyl, cycloalkyl, heteroalkyl, or heterocycloalkyl;
W is O, NH, or NMe; and
R₉ is an alkyl or a heteroalkyl, with the proviso that R₉ is not H.

18. The compound of claim 17, or a pharmaceutically acceptable salt thereof, having a structure of:

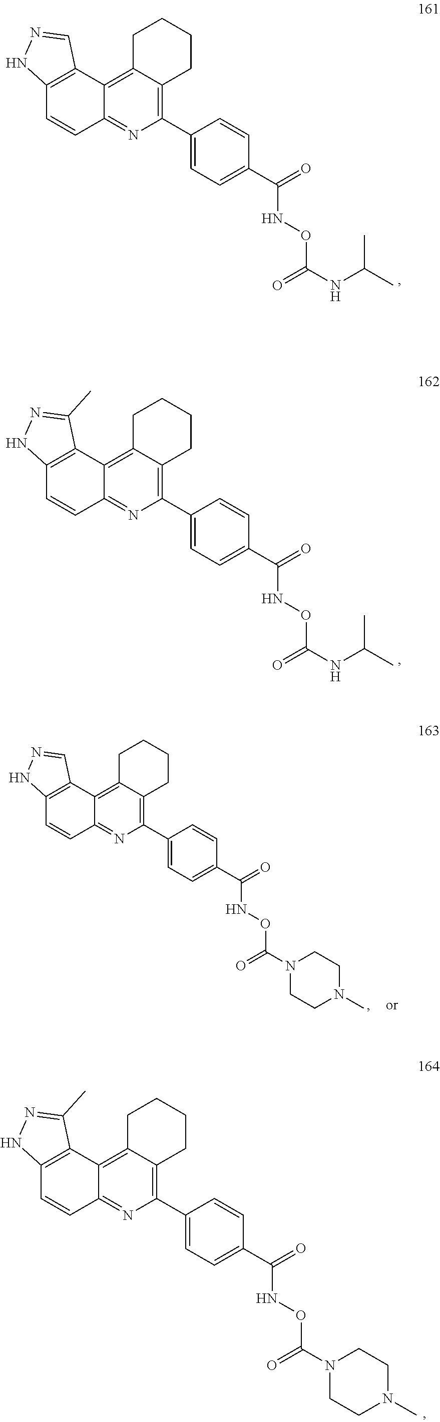 [SMILES: CC(C)NC(=O)ONC(=O)c1ccc(-c2nc3ccc4[nH]ncc4c3c3c2CCCC3)cc1 ; Cc1n[nH]c2ccc3nc(-c4ccc(C(=O)NOC(=O)NC(C)C)cc4)c4c(c3c12)CCCC4 ; CN1CCN(C(=O)ONC(=O)c2ccc(-c3nc4ccc5[nH]ncc5c4c4c3CCCC4)cc2)CC1 ; Cc1n[nH]c2ccc3nc(-c4ccc(C(=O)NOC(=O)N5CCN(C)CC5)cc4)c4c(c3c12)CCCC4]

19. (canceled)

20. The compound of claim 3, or a pharmaceutically acceptable salt thereof, having a structure of

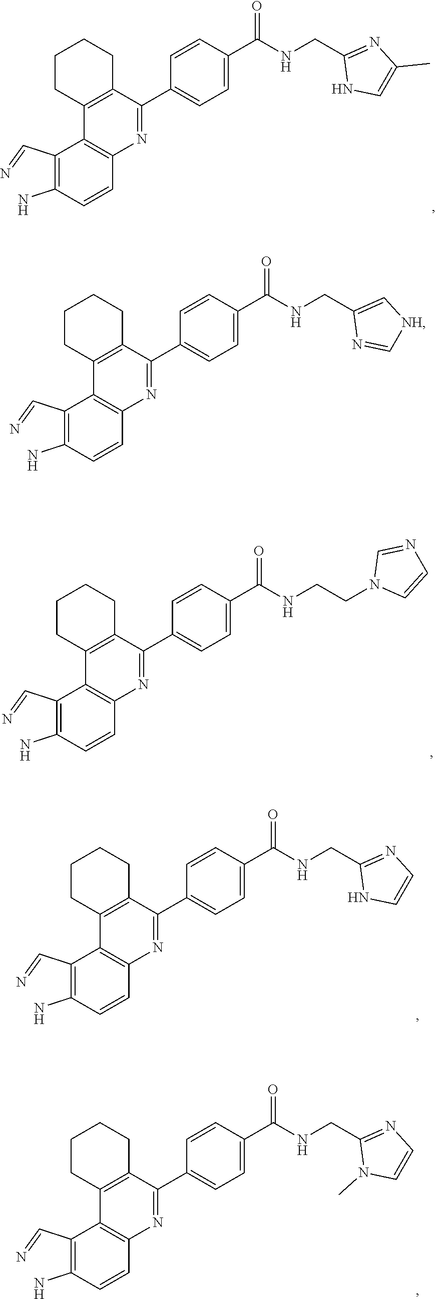 [SMILES: Cc1c[nH]c(CNC(=O)c2ccc(-c3nc4ccc(N)c(C=N)c4c4c3CCCC4)cc2)n1 ; N=Cc1c(N)ccc2nc(-c3ccc(C(=O)NCc4c[nH]cn4)cc3)c3c(c12)CCCC3 ; N=Cc1c(N)ccc2nc(-c3ccc(C(=O)NCCn4ccnc4)cc3)c3c(c12)CCCC3 ; N=Cc1c(N)ccc2nc(-c3ccc(C(=O)NCc4ncc[nH]4)cc3)c3c(c12)CCCC3 ; Cn1ccnc1CNC(=O)c1ccc(-c2nc3ccc(N)c(C=N)c3c3c2CCCC3)cc1]

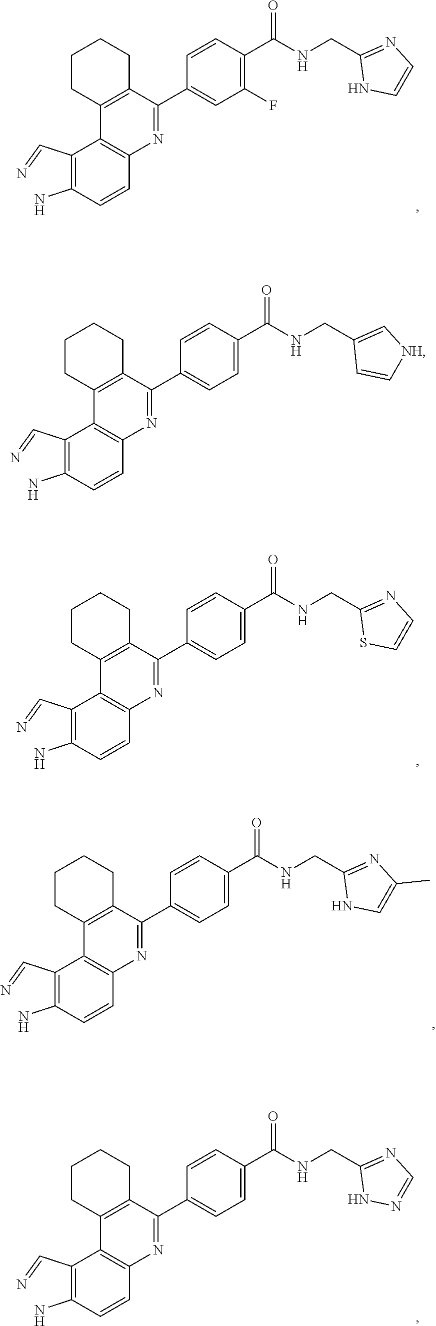 [SMILES: N=Cc1c(N)ccc2nc(-c3ccc(C(=O)NCc4ncc[nH]4)c(F)c3)c3c(c12)CCCC3 ; N=Cc1c(N)ccc2nc(-c3ccc(C(=O)NCc4cc[nH]c4)cc3)c3c(c12)CCCC3 ; N=Cc1c(N)ccc2nc(-c3ccc(C(=O)NCc4nccs4)cc3)c3c(c12)CCCC3 ; Cc1c[nH]c(CNC(=O)c2ccc(-c3nc4ccc(N)c(C=N)c4c4c3CCCC4)cc2)n1 ; N=Cc1c(N)ccc2nc(-c3ccc(C(=O)NCc4ncn[nH]4)cc3)c3c(c12)CCCC3]

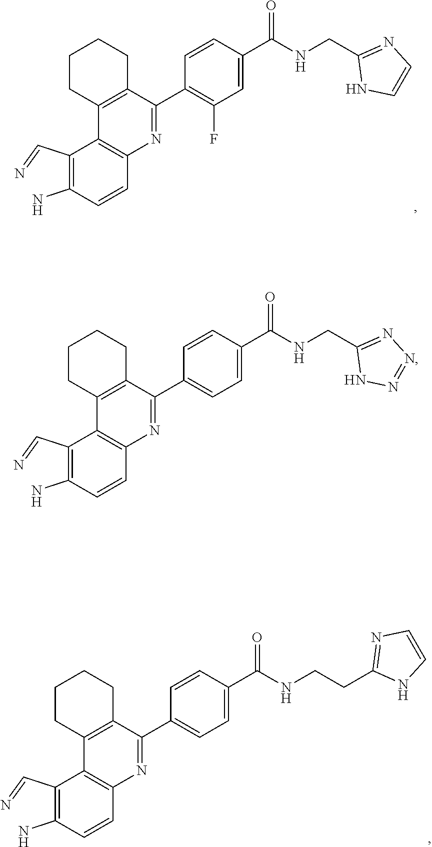 [SMILES: N=Cc1c(N)ccc2nc(-c3ccc(C(=O)NCc4ncc[nH]4)cc3F)c3c(c12)CCCC3 ; N=Cc1c(N)ccc2nc(-c3ccc(C(=O)NCc4nnn[nH]4)cc3)c3c(c12)CCCC3 ; N=Cc1c(N)ccc2nc(-c3ccc(C(=O)NCCc4ncc[nH]4)cc3)c3c(c12)CCCC3]

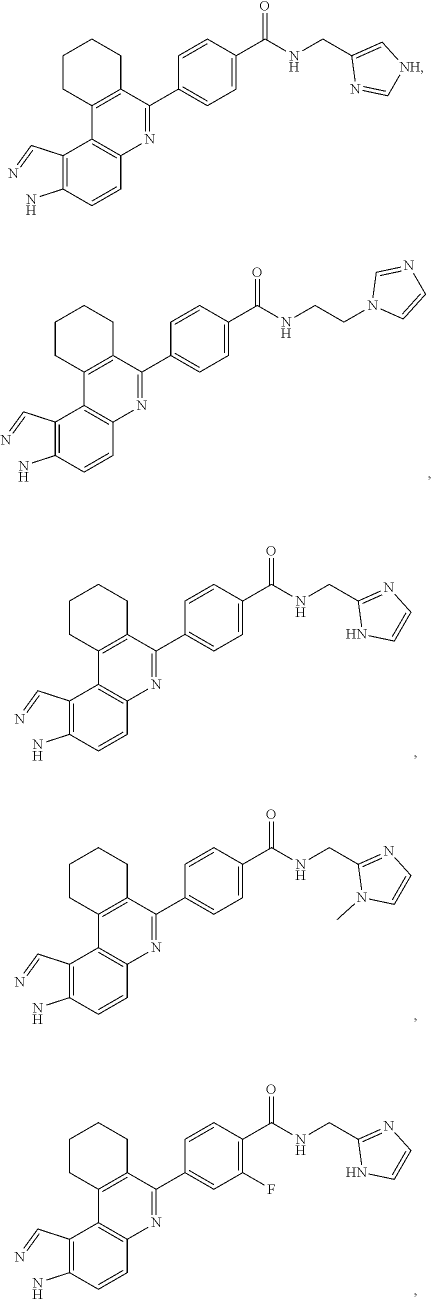 [SMILES: N=Cc1c(N)ccc2nc(-c3ccc(C(=O)NCc4c[nH]cn4)cc3)c3c(c12)CCCC3 ; N=Cc1c(N)ccc2nc(-c3ccc(C(=O)NCCn4ccnc4)cc3)c3c(c12)CCCC3 ; N=Cc1c(N)ccc2nc(-c3ccc(C(=O)NCc4ncc[nH]4)cc3)c3c(c12)CCCC3 ; Cn1ccnc1CNC(=O)c1ccc(-c2nc3ccc(N)c(C=N)c3c3c2CCCC3)cc1 ; N=Cc1c(N)ccc2nc(-c3ccc(C(=O)NCc4ncc[nH]4)c(F)c3)c3c(c12)CCCC3]

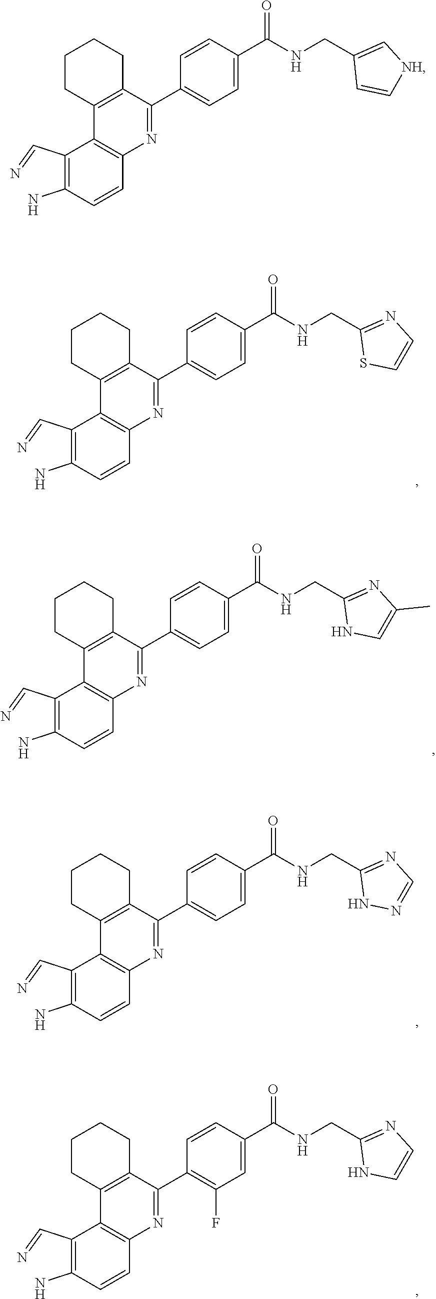 [SMILES: N=Cc1c(N)ccc2nc(-c3ccc(C(=O)NCc4cc[nH]c4)cc3)c3c(c12)CCCC3 ; N=Cc1c(N)ccc2nc(-c3ccc(C(=O)NCc4nccs4)cc3)c3c(c12)CCCC3 ; Cc1c[nH]c(CNC(=O)c2ccc(-c3nc4ccc(N)c(C=N)c4c4c3CCCC4)cc2)n1 ; N=Cc1c(N)ccc2nc(-c3ccc(C(=O)NCc4ncn[nH]4)cc3)c3c(c12)CCCC3 ; N=Cc1c(N)ccc2nc(-c3ccc(C(=O)NCc4ncc[nH]4)cc3F)c3c(c12)CCCC3]

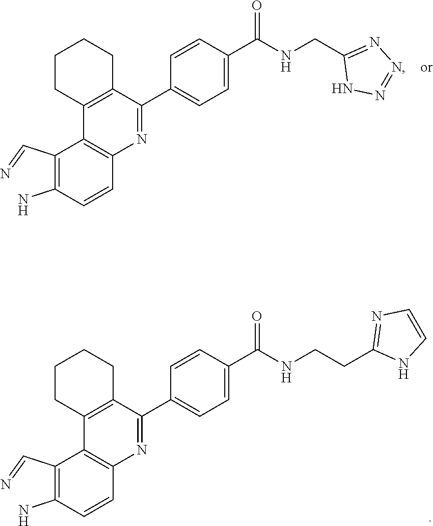 [SMILES: N=Cc1c(N)ccc2nc(-c3ccc(C(=O)NCc4nnn[nH]4)cc3)c3c(c12)CCCC3 ; N=Cc1c(N)ccc2nc(-c3ccc(C(=O)NCCc4ncc[nH]4)cc3)c3c(c12)CCCC3]

21. The compound of claim 1 having a structure of:

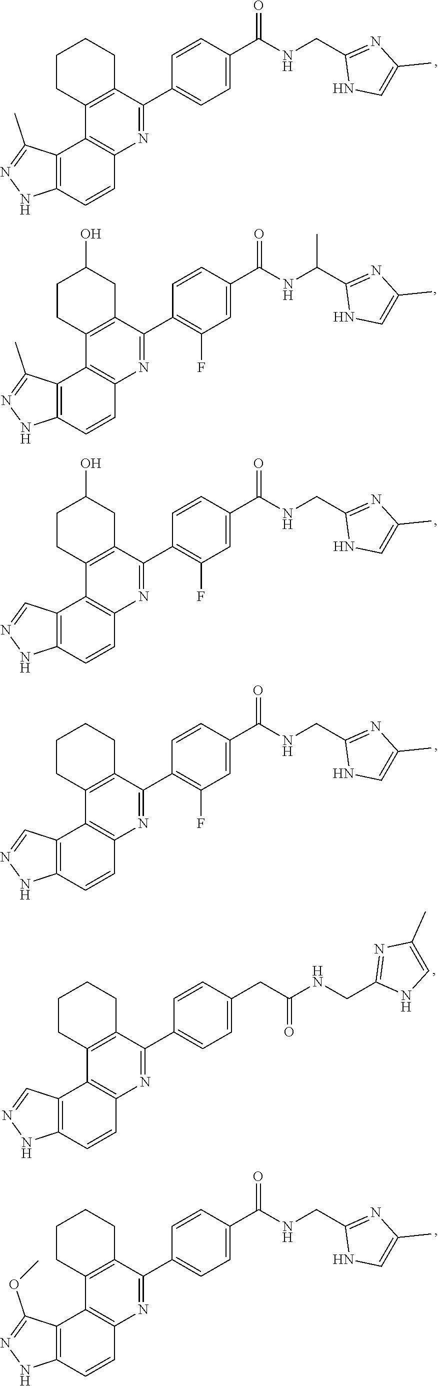

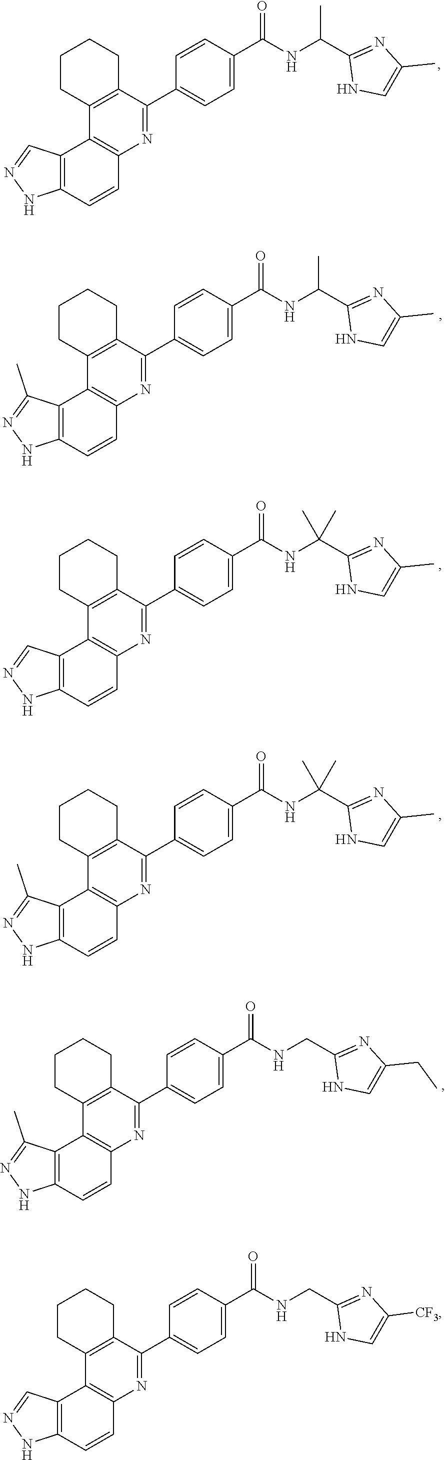 [SMILES: Cc1c[nH]c(C(C)NC(=O)c2ccc(-c3nc4ccc5[nH]ncc5c4c4c3CCCC4)cc2)n1 ; Cc1c[nH]c(C(C)NC(=O)c2ccc(-c3nc4ccc5[nH]nc(C)c5c4c4c3CCCC4)cc2)n1 ; Cc1c[nH]c(C(C)(C)NC(=O)c2ccc(-c3nc4ccc5[nH]ncc5c4c4c3CCCC4)cc2)n1 ; Cc1c[nH]c(C(C)(C)NC(=O)c2ccc(-c3nc4ccc5[nH]nc(C)c5c4c4c3CCCC4)cc2)n1 ; CCc1c[nH]c(CNC(=O)c2ccc(-c3nc4ccc5[nH]nc(C)c5c4c4c3CCCC4)cc2)n1 ; O=C(NCc1nc(C(F)(F)F)c[nH]1)c1ccc(-c2nc3ccc4[nH]ncc4c3c3c2CCCC3)cc1]

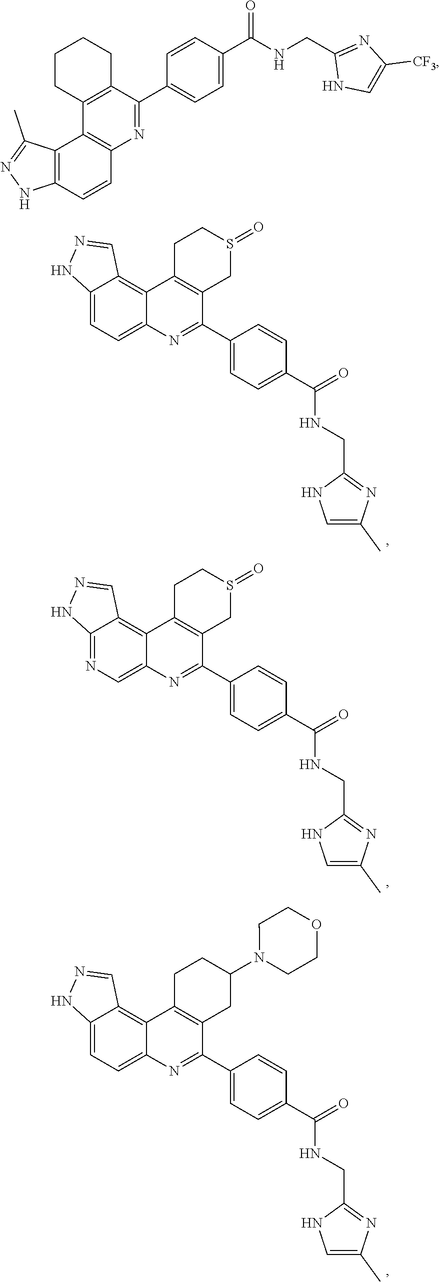

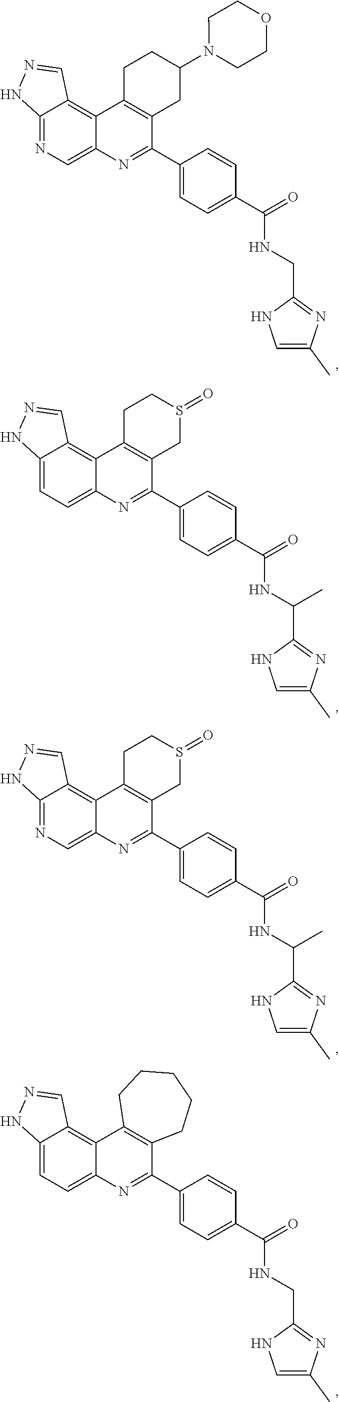 [SMILES: Cc1c[nH]c(CNC(=O)c2ccc(-c3nc4cnc5[nH]ncc5c4c4c3CC(N3CCOCC3)CC4)cc2)n1 ; Cc1c[nH]c(C(C)NC(=O)c2ccc(-c3nc4ccc5[nH]ncc5c4c4c3CS(=O)CC4)cc2)n1 ; Cc1c[nH]c(C(C)NC(=O)c2ccc(-c3nc4cnc5[nH]ncc5c4c4c3CS(=O)CC4)cc2)n1 ; Cc1c[nH]c(CNC(=O)c2ccc(-c3nc4ccc5[nH]ncc5c4c4c3CCCCC4)cc2)n1]

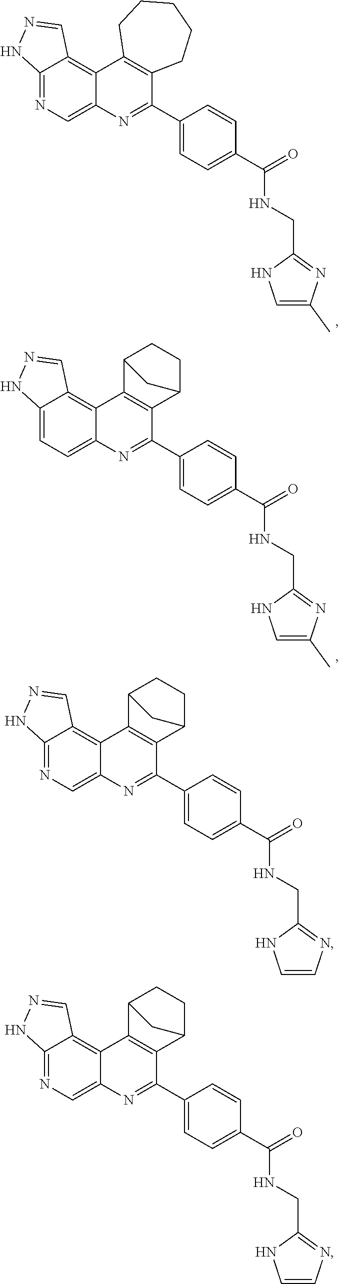 [SMILES: Cc1c[nH]c(CNC(=O)c2ccc(-c3nc4cnc5[nH]ncc5c4c4c3CCCCC4)cc2)n1 ; Cc1c[nH]c(CNC(=O)c2ccc(-c3nc4ccc5[nH]ncc5c4c4c3C3CCC4C3)cc2)n1 ; O=C(NCc1ncc[nH]1)c1ccc(-c2nc3cnc4[nH]ncc4c3c3c2C2CCC3C2)cc1 ; O=C(NCc1ncc[nH]1)c1ccc(-c2nc3cnc4[nH]ncc4c3c3c2C2CCC3C2)cc1]

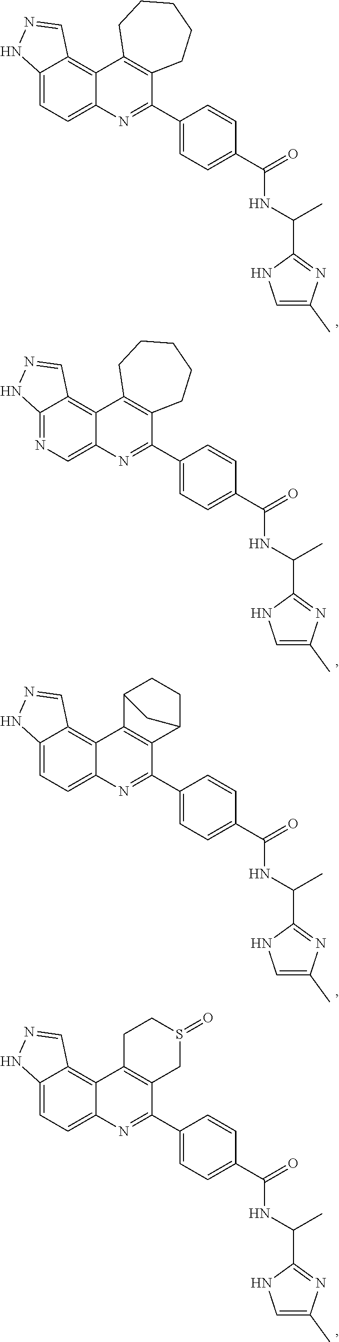 [SMILES: Cc1c[nH]c(C(C)NC(=O)c2ccc(-c3nc4ccc5[nH]ncc5c4c4c3CCCCC4)cc2)n1 ; Cc1c[nH]c(C(C)NC(=O)c2ccc(-c3nc4cnc5[nH]ncc5c4c4c3CCCCC4)cc2)n1 ; Cc1c[nH]c(C(C)NC(=O)c2ccc(-c3nc4ccc5[nH]ncc5c4c4c3C3CCC4C3)cc2)n1 ; Cc1c[nH]c(C(C)NC(=O)c2ccc(-c3nc4ccc5[nH]ncc5c4c4c3CS(=O)CC4)cc2)n1]

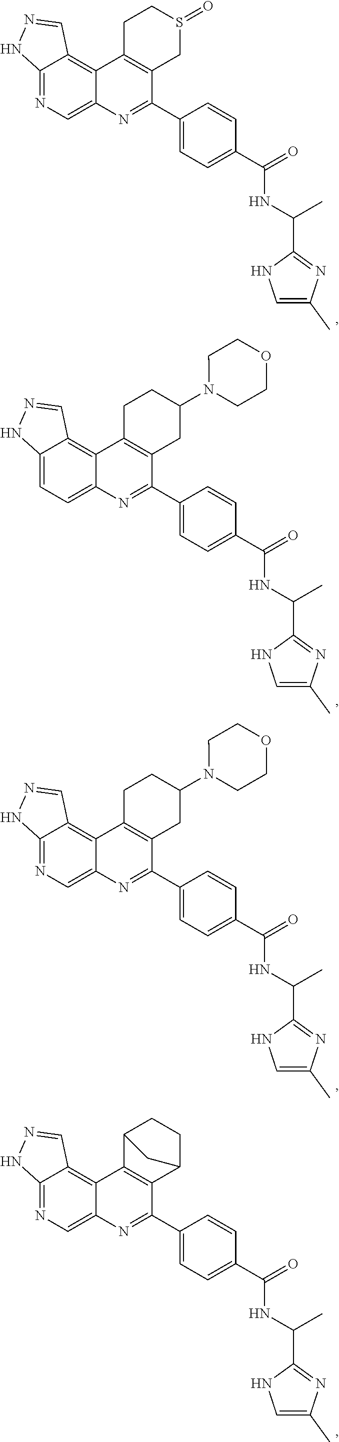 [SMILES: Cc1c[nH]c(C(C)NC(=O)c2ccc(-c3nc4cnc5[nH]ncc5c4c4c3CS(=O)CC4)cc2)n1 ; Cc1c[nH]c(C(C)NC(=O)c2ccc(-c3nc4ccc5[nH]ncc5c4c4c3CC(N3CCOCC3)CC4)cc2)n1 ; Cc1c[nH]c(C(C)NC(=O)c2ccc(-c3nc4cnc5[nH]ncc5c4c4c3CC(N3CCOCC3)CC4)cc2)n1 ; Cc1c[nH]c(C(C)NC(=O)c2ccc(-c3nc4cnc5[nH]ncc5c4c4c3C3CCC4C3)cc2)n1]

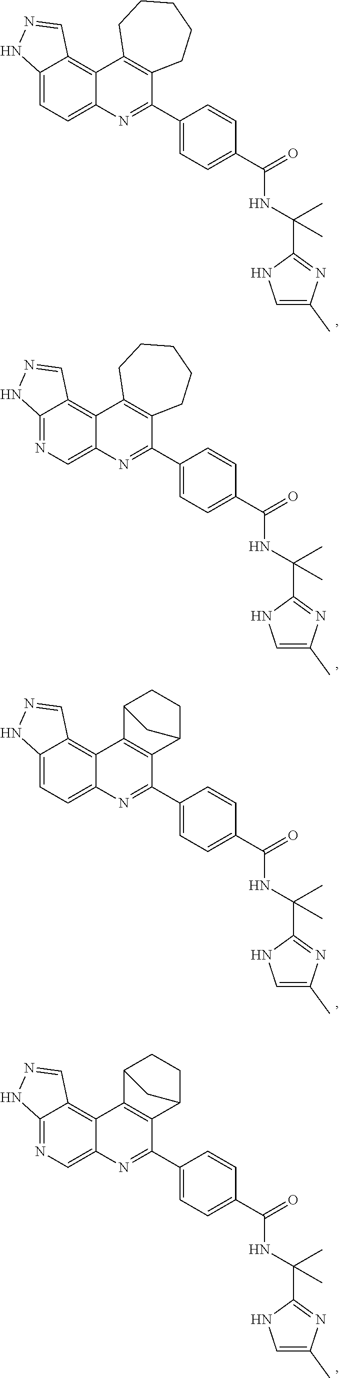 [SMILES: Cc1c[nH]c(C(C)(C)NC(=O)c2ccc(-c3nc4ccc5[nH]ncc5c4c4c3CCCCC4)cc2)n1 ; Cc1c[nH]c(C(C)(C)NC(=O)c2ccc(-c3nc4cnc5[nH]ncc5c4c4c3CCCCC4)cc2)n1 ; Cc1c[nH]c(C(C)(C)NC(=O)c2ccc(-c3nc4ccc5[nH]ncc5c4c4c3C3CCC4C3)cc2)n1 ; Cc1c[nH]c(C(C)(C)NC(=O)c2ccc(-c3nc4cnc5[nH]ncc5c4c4c3C3CCC4C3)cc2)n1]

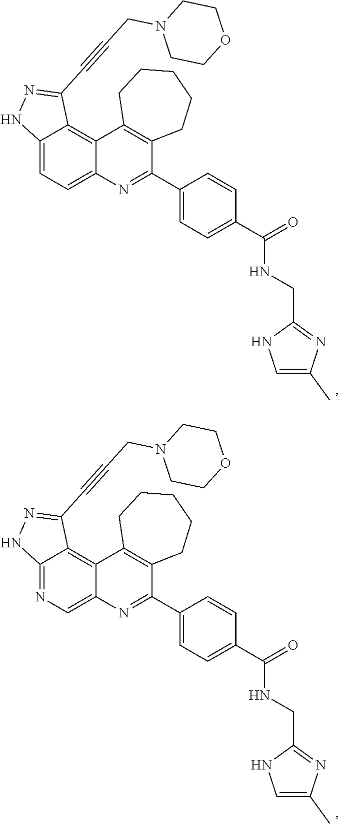 [SMILES: Cc1c[nH]c(CNC(=O)c2ccc(-c3nc4ccc5[nH]nc(C#CCN6CCOCC6)c5c4c4c3CCCCC4)cc2)n1 ; Cc1c[nH]c(CNC(=O)c2ccc(-c3nc4cnc5[nH]nc(C#CCN6CCOCC6)c5c4c4c3CCCCC4)cc2)n1]

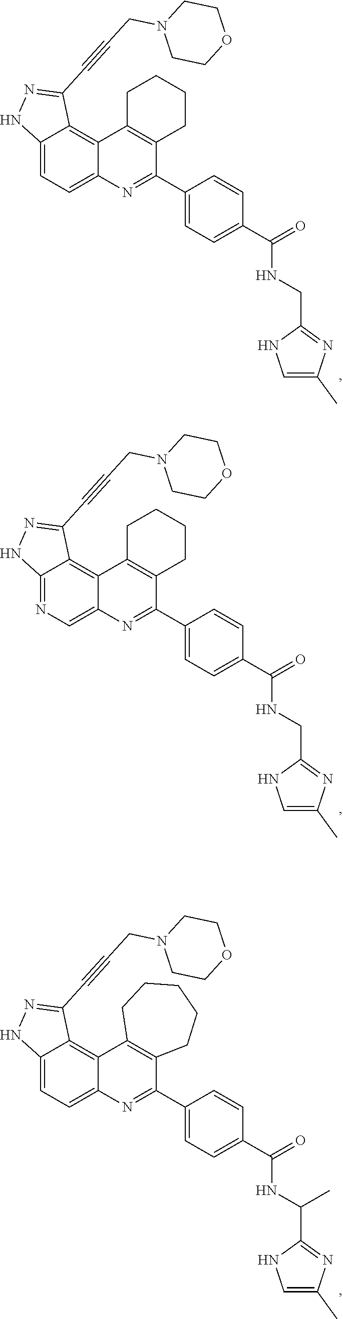 [SMILES: Cc1c[nH]c(CNC(=O)c2ccc(-c3nc4ccc5[nH]nc(C#CCN6CCOCC6)c5c4c4c3CCCC4)cc2)n1 ; Cc1c[nH]c(CNC(=O)c2ccc(-c3nc4cnc5[nH]nc(C#CCN6CCOCC6)c5c4c4c3CCCC4)cc2)n1 ; Cc1c[nH]c(C(C)NC(=O)c2ccc(-c3nc4ccc5[nH]nc(C#CCN6CCOCC6)c5c4c4c3CCCCC4)cc2)n1]

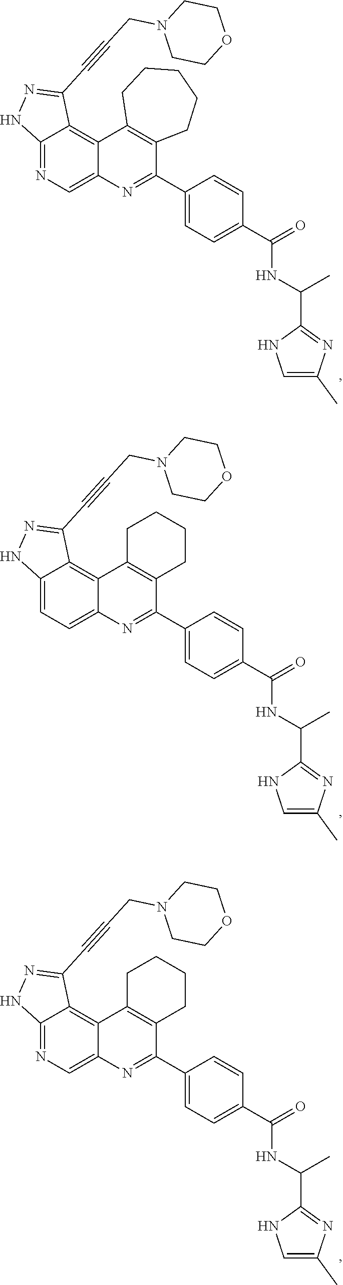 [SMILES: Cc1c[nH]c(C(C)NC(=O)c2ccc(-c3nc4cnc5[nH]nc(C#CCN6CCOCC6)c5c4c4c3CCCCC4)cc2)n1 ; Cc1c[nH]c(C(C)NC(=O)c2ccc(-c3nc4ccc5[nH]nc(C#CCN6CCOCC6)c5c4c4c3CCCC4)cc2)n1 ; Cc1c[nH]c(C(C)NC(=O)c2ccc(-c3nc4cnc5[nH]nc(C#CCN6CCOCC6)c5c4c4c3CCCC4)cc2)n1]

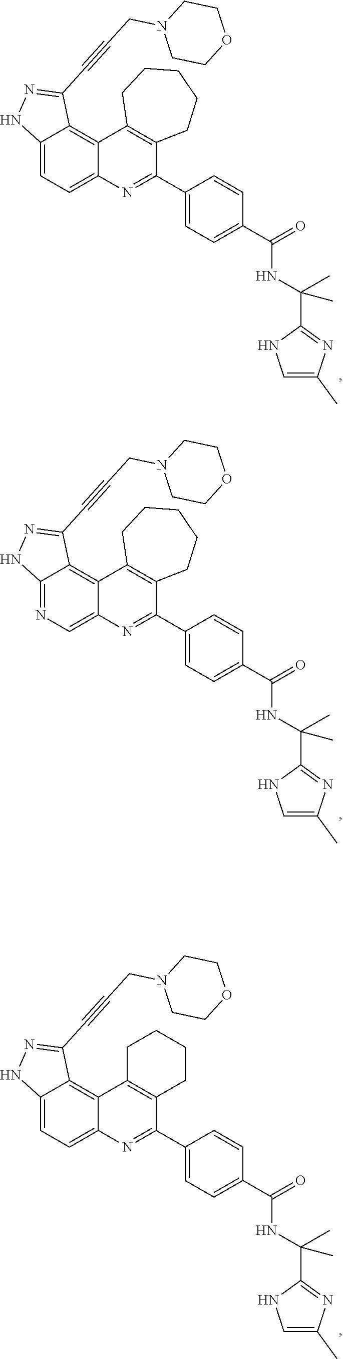 [SMILES: Cc1c[nH]c(C(C)(C)NC(=O)c2ccc(-c3nc4ccc5[nH]nc(C#CCN6CCOCC6)c5c4c4c3CCCCC4)cc2)n1 ; Cc1c[nH]c(C(C)(C)NC(=O)c2ccc(-c3nc4cnc5[nH]nc(C#CCN6CCOCC6)c5c4c4c3CCCCC4)cc2)n1 ; Cc1c[nH]c(C(C)(C)NC(=O)c2ccc(-c3nc4ccc5[nH]nc(C#CCN6CCOCC6)c5c4c4c3CCCC4)cc2)n1]

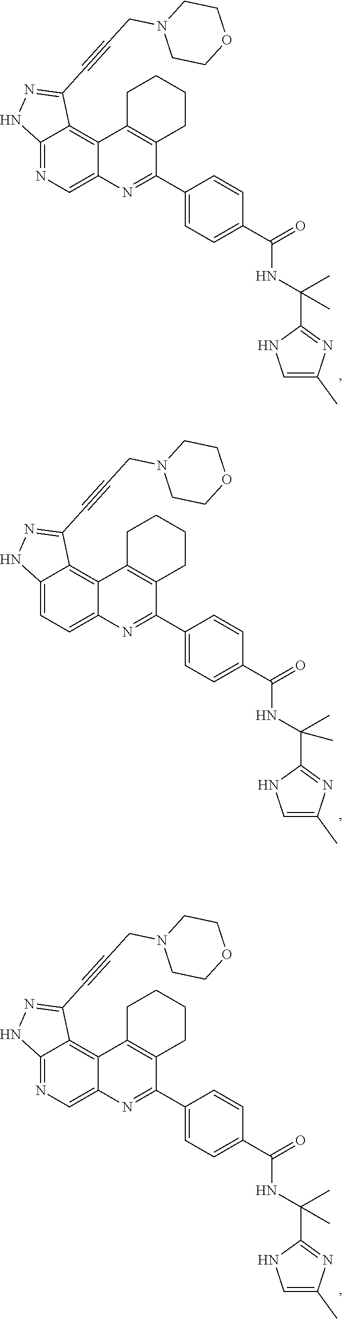 [SMILES: Cc1c[nH]c(C(C)(C)NC(=O)c2ccc(-c3nc4cnc5[nH]nc(C#CCN6CCOCC6)c5c4c4c3CCCC4)cc2)n1 ; Cc1c[nH]c(C(C)(C)NC(=O)c2ccc(-c3nc4ccc5[nH]nc(C#CCN6CCOCC6)c5c4c4c3CCCC4)cc2)n1 ; Cc1c[nH]c(C(C)(C)NC(=O)c2ccc(-c3nc4cnc5[nH]nc(C#CCN6CCOCC6)c5c4c4c3CCCC4)cc2)n1]

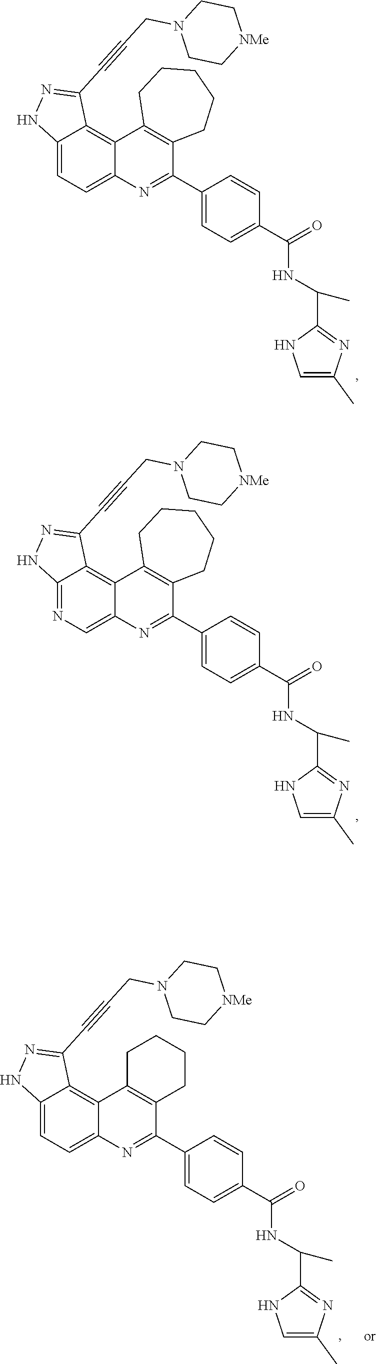 [SMILES: Cc1c[nH]c(C(C)NC(=O)c2ccc(-c3nc4ccc5[nH]nc(C#CCN6CCN(C)CC6)c5c4c4c3CCCCC4)cc2)n1 ; Cc1c[nH]c(C(C)NC(=O)c2ccc(-c3nc4cnc5[nH]nc(C#CCN6CCN(C)CC6)c5c4c4c3CCCCC4)cc2)n1 ; Cc1c[nH]c(C(C)NC(=O)c2ccc(-c3nc4ccc5[nH]nc(C#CCN6CCN(C)CC6)c5c4c4c3CCCC4)cc2)n1]

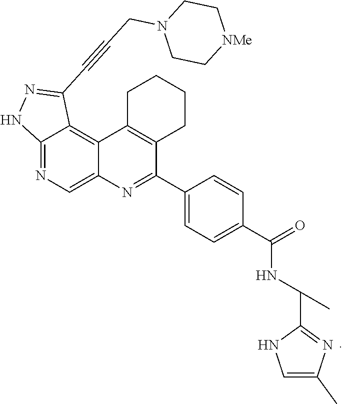 [SMILES: Cc1c[nH]c(C(C)NC(=O)c2ccc(-c3nc4cnc5[nH]nc(C#CCN6CCN(C)CC6)c5c4c4c3CCCC4)cc2)n1]

22. (canceled)

23. The compound of claim 1 having a structure of:

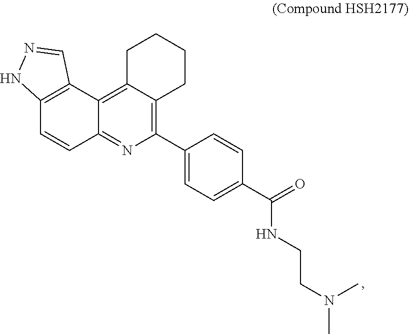 [SMILES: CN(C)CCNC(=O)c1ccc(-c2nc3ccc4[nH]ncc4c3c3c2CCCC3)cc1]

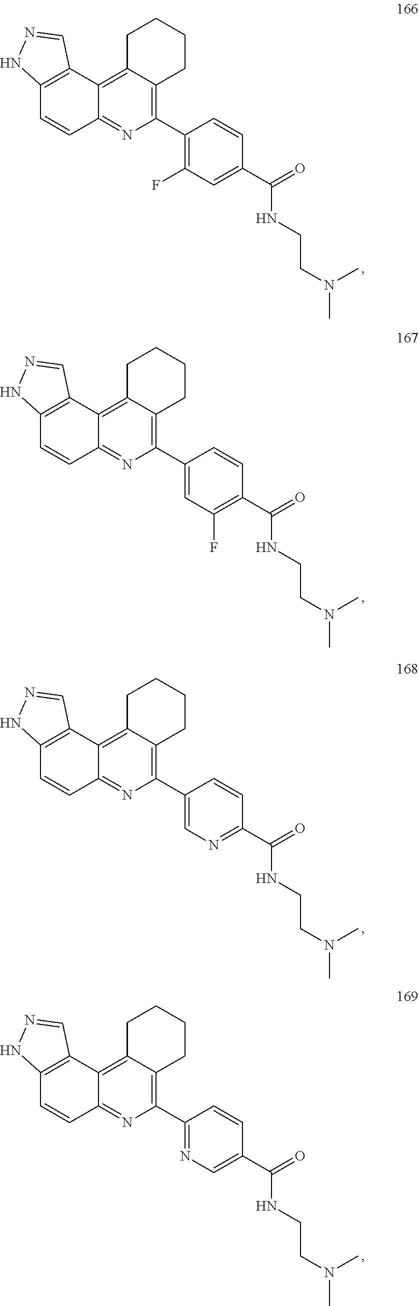 [SMILES: CN(C)CCNC(=O)c1ccc(-c2nc3ccc4[nH]ncc4c3c3c2CCCC3)c(F)c1 ; CN(C)CCNC(=O)c1ccc(-c2nc3ccc4[nH]ncc4c3c3c2CCCC3)cc1F ; CN(C)CCNC(=O)c1ccc(-c2nc3ccc4[nH]ncc4c3c3c2CCCC3)cn1 ; CN(C)CCNC(=O)c1ccc(-c2nc3ccc4[nH]ncc4c3c3c2CCCC3)nc1]

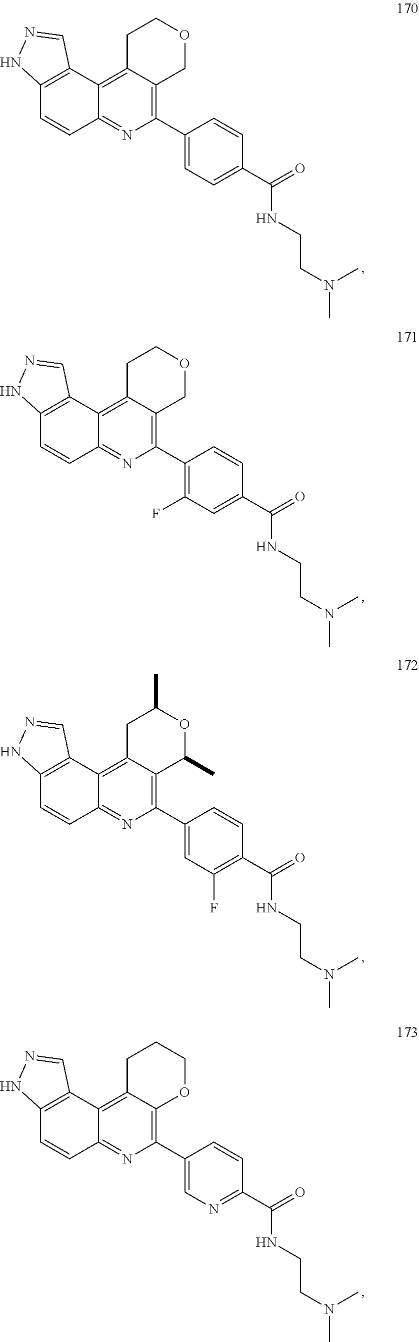 [SMILES: CN(C)CCNC(=O)c1ccc(-c2nc3ccc4[nH]ncc4c3c3c2COCC3)cc1 ; CN(C)CCNC(=O)c1ccc(-c2nc3ccc4[nH]ncc4c3c3c2COCC3)c(F)c1 ; C[C@@H]1Cc2c(c(-c3ccc(C(=O)NCCN(C)C)c(F)c3)nc3ccc4[nH]ncc4c23)[C@H](C)O1 ; CN(C)CCNC(=O)c1ccc(-c2nc3ccc4[nH]ncc4c3c3c2OCCC3)cn1]

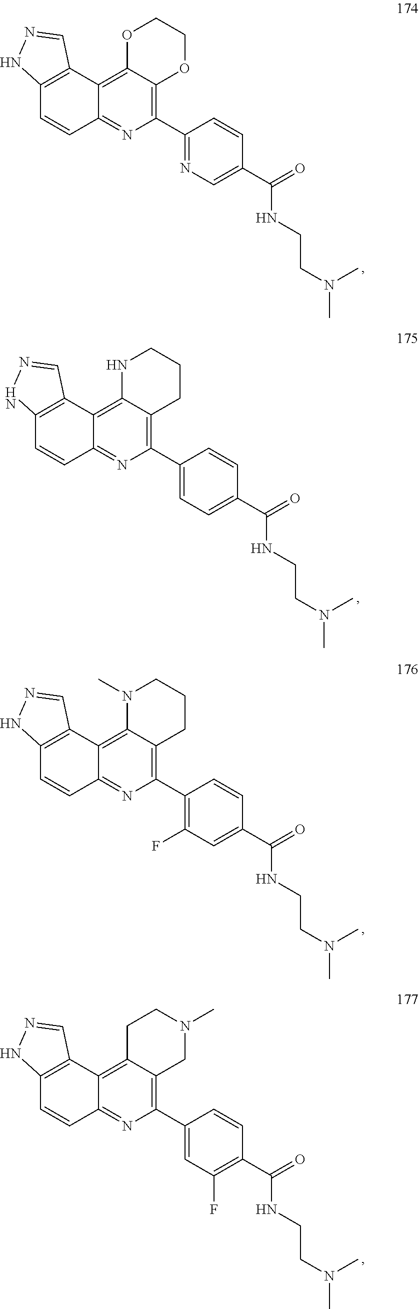 [SMILES: CN(C)CCNC(=O)c1ccc(-c2nc3ccc4[nH]ncc4c3c3c2OCCO3)nc1 ; CN(C)CCNC(=O)c1ccc(-c2nc3ccc4[nH]ncc4c3c3c2CCCN3)cc1 ; CN(C)CCNC(=O)c1ccc(-c2nc3ccc4[nH]ncc4c3c3c2CCCN3C)c(F)c1 ; CN(C)CCNC(=O)c1ccc(-c2nc3ccc4[nH]ncc4c3c3c2CN(C)CC3)cc1F]

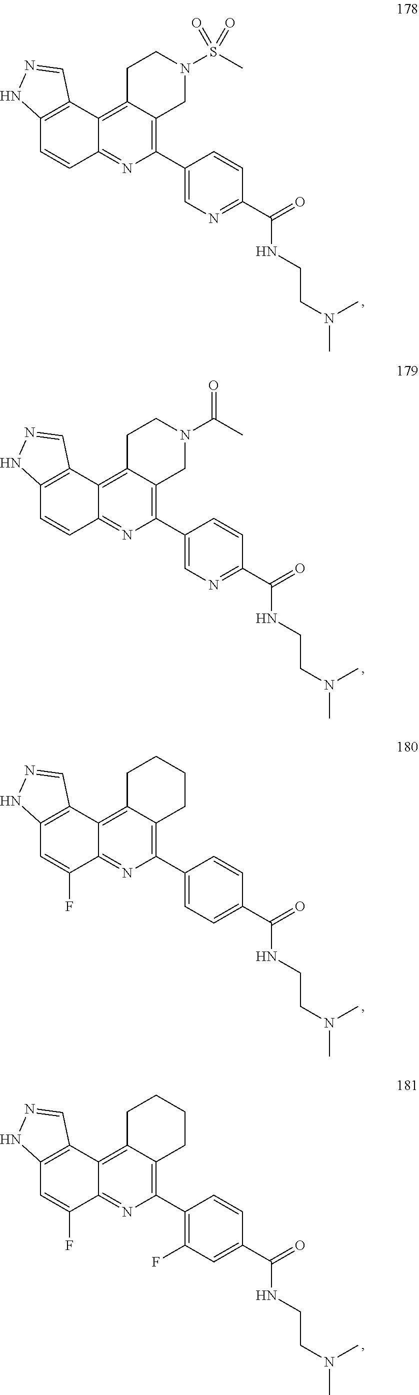 [SMILES: CN(C)CCNC(=O)c1ccc(-c2nc3ccc4[nH]ncc4c3c3c2CN(S(C)(=O)=O)CC3)cn1 ; CC(=O)N1CCc2c(c(-c3ccc(C(=O)NCCN(C)C)nc3)nc3ccc4[nH]ncc4c23)C1 ; CN(C)CCNC(=O)c1ccc(-c2nc3c(F)cc4[nH]ncc4c3c3c2CCCC3)cc1 ; CN(C)CCNC(=O)c1ccc(-c2nc3c(F)cc4[nH]ncc4c3c3c2CCCC3)c(F)c1]

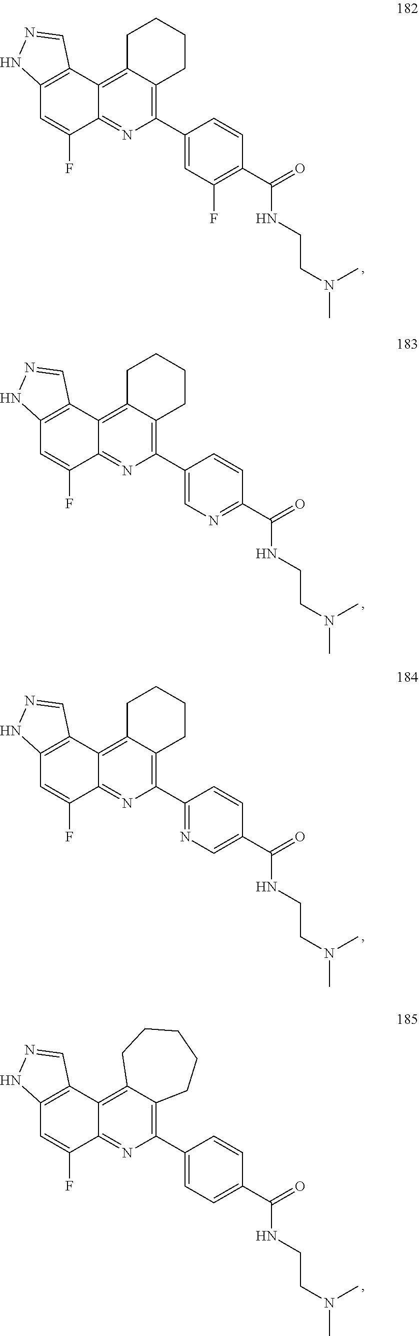 [SMILES: CN(C)CCNC(=O)c1ccc(-c2nc3c(F)cc4[nH]ncc4c3c3c2CCCC3)cc1F ; CN(C)CCNC(=O)c1ccc(-c2nc3c(F)cc4[nH]ncc4c3c3c2CCCC3)cn1 ; CN(C)CCNC(=O)c1ccc(-c2nc3c(F)cc4[nH]ncc4c3c3c2CCCC3)nc1 ; CN(C)CCNC(=O)c1ccc(-c2nc3c(F)cc4[nH]ncc4c3c3c2CCCCC3)cc1]

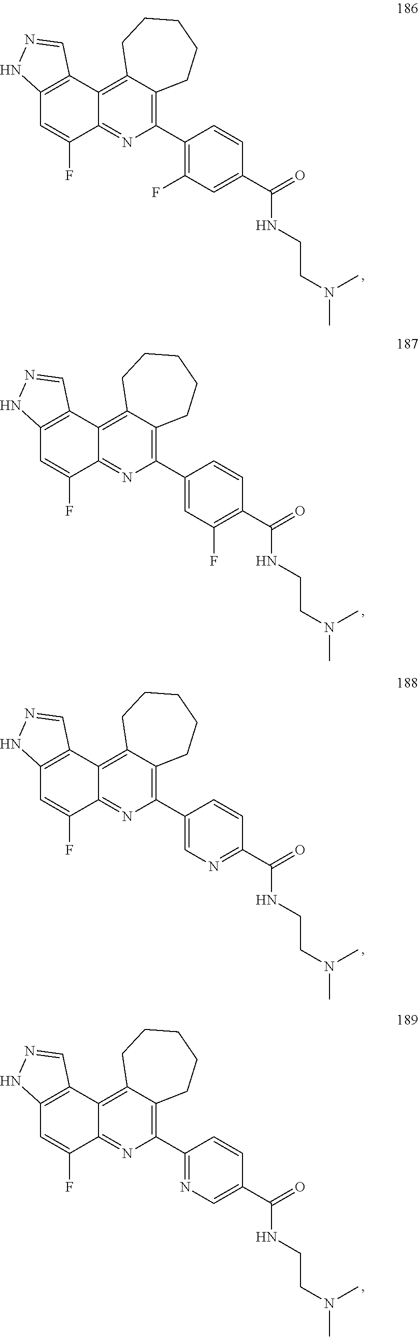 [SMILES: CN(C)CCNC(=O)c1ccc(-c2nc3c(F)cc4[nH]ncc4c3c3c2CCCCC3)c(F)c1 ; CN(C)CCNC(=O)c1ccc(-c2nc3c(F)cc4[nH]ncc4c3c3c2CCCCC3)cc1F ; CN(C)CCNC(=O)c1ccc(-c2nc3c(F)cc4[nH]ncc4c3c3c2CCCCC3)cn1 ; CN(C)CCNC(=O)c1ccc(-c2nc3c(F)cc4[nH]ncc4c3c3c2CCCCC3)nc1]

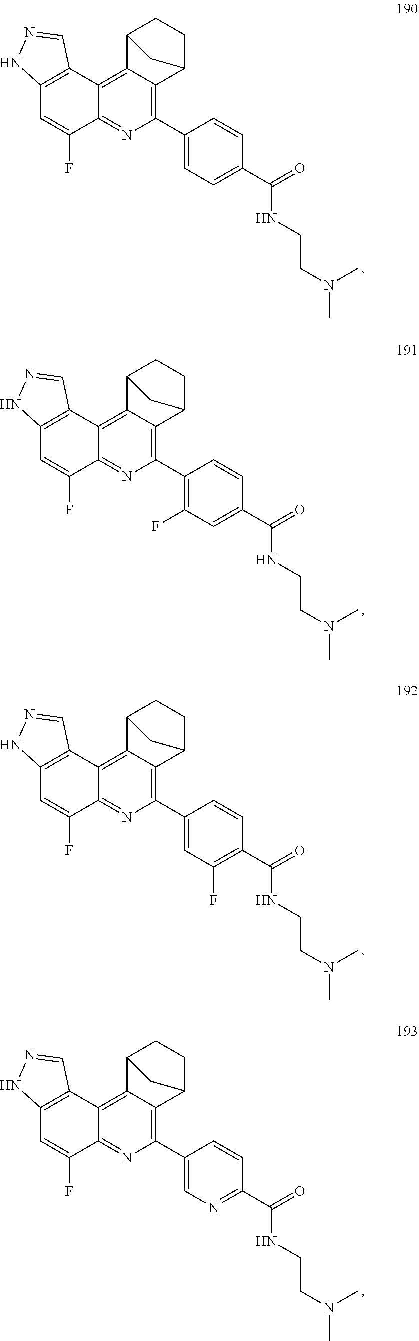 [SMILES: CN(C)CCNC(=O)c1ccc(-c2nc3c(F)cc4[nH]ncc4c3c3c2C2CCC3C2)cc1 ; CN(C)CCNC(=O)c1ccc(-c2nc3c(F)cc4[nH]ncc4c3c3c2C2CCC3C2)c(F)c1 ; CN(C)CCNC(=O)c1ccc(-c2nc3c(F)cc4[nH]ncc4c3c3c2C2CCC3C2)cc1F ; CN(C)CCNC(=O)c1ccc(-c2nc3c(F)cc4[nH]ncc4c3c3c2C2CCC3C2)cn1]

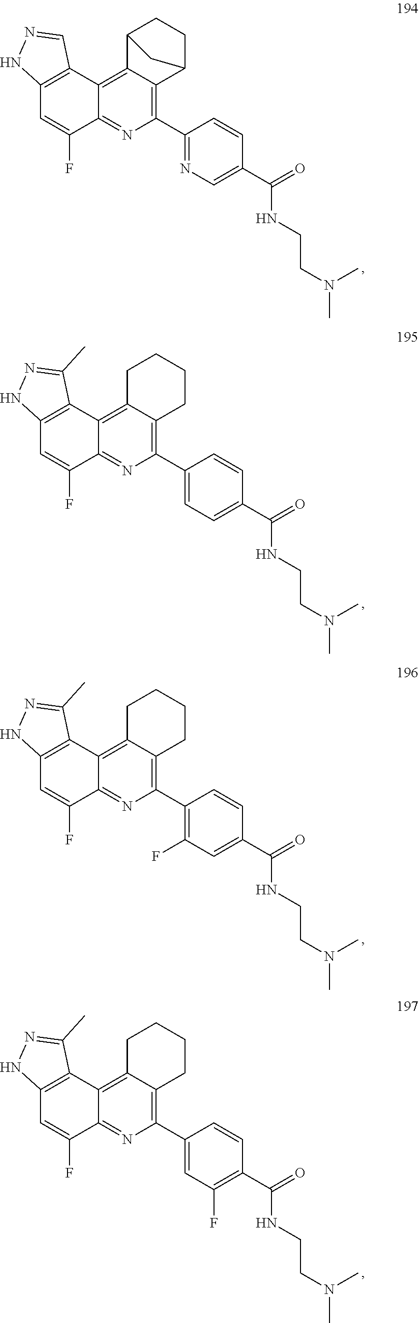 [SMILES: CN(C)CCNC(=O)c1ccc(-c2nc3c(F)cc4[nH]ncc4c3c3c2C2CCC3C2)nc1 ; Cc1n[nH]c2cc(F)c3nc(-c4ccc(C(=O)NCCN(C)C)cc4)c4c(c3c12)CCCC4 ; Cc1n[nH]c2cc(F)c3nc(-c4ccc(C(=O)NCCN(C)C)cc4F)c4c(c3c12)CCCC4 ; Cc1n[nH]c2cc(F)c3nc(-c4ccc(C(=O)NCCN(C)C)c(F)c4)c4c(c3c12)CCCC4]

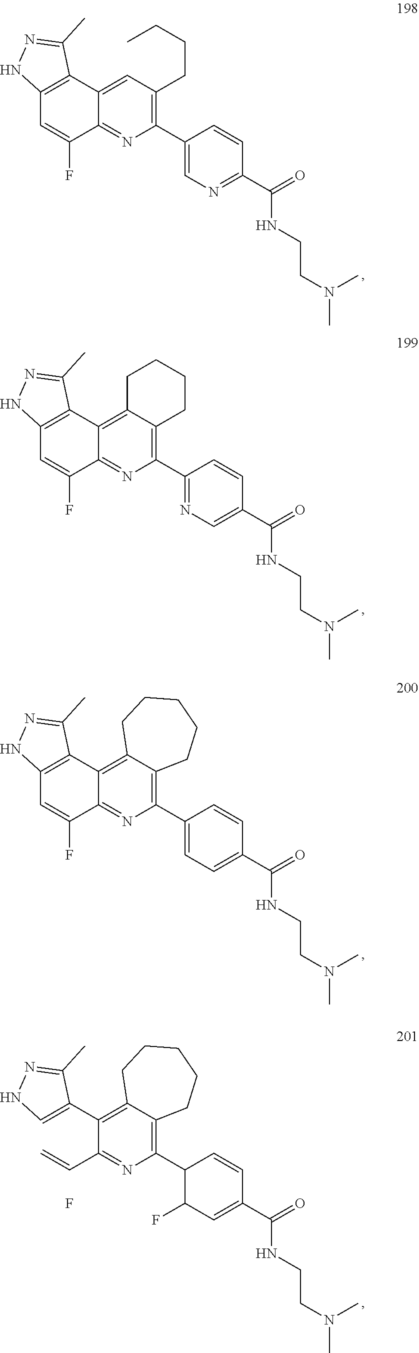

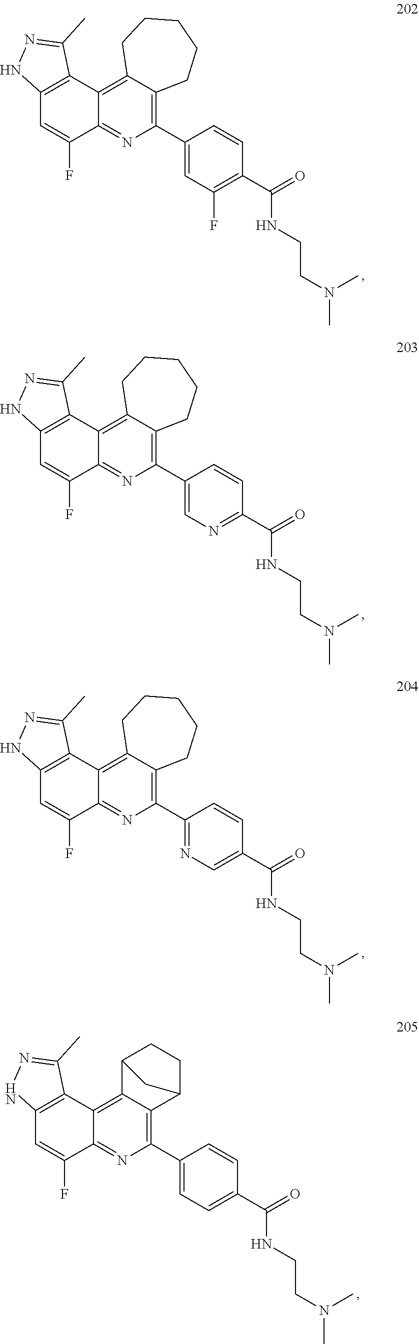 [SMILES: Cc1n[nH]c2cc(F)c3nc(-c4ccc(C(=O)NCCN(C)C)c(F)c4)c4c(c3c12)CCCCC4 ; Cc1n[nH]c2cc(F)c3nc(-c4ccc(C(=O)NCCN(C)C)nc4)c4c(c3c12)CCCCC4 ; Cc1n[nH]c2cc(F)c3nc(-c4ccc(C(=O)NCCN(C)C)cn4)c4c(c3c12)CCCCC4 ; Cc1n[nH]c2cc(F)c3nc(-c4ccc(C(=O)NCCN(C)C)cc4)c4c(c3c12)C1CCC4C1]

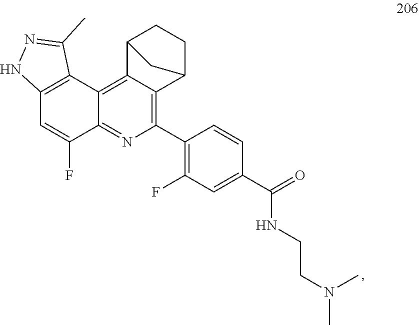 [SMILES: Cc1n[nH]c2cc(F)c3nc(-c4ccc(C(=O)NCCN(C)C)cc4F)c4c(c3c12)C1CCC4C1]

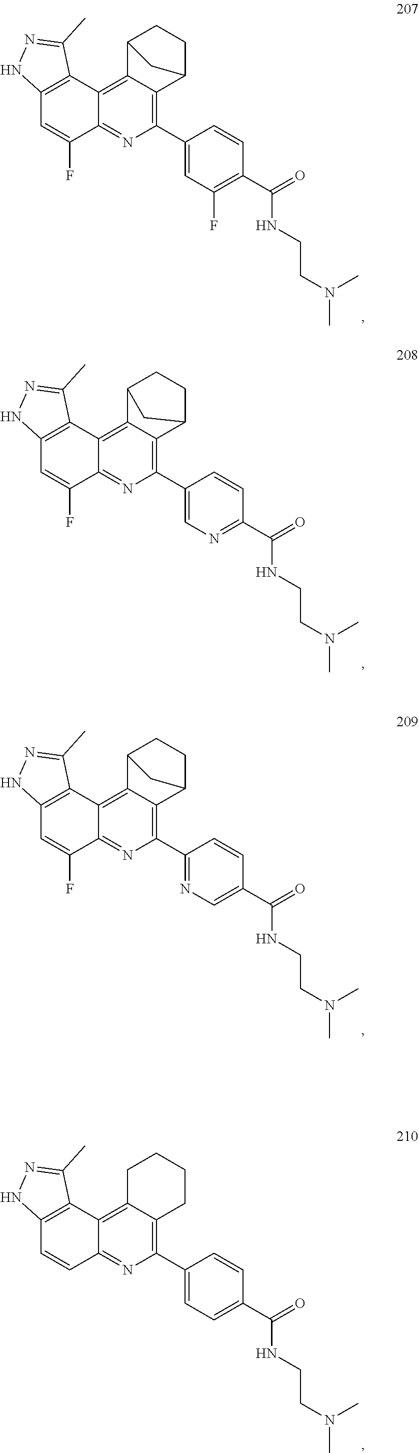 [SMILES: Cc1n[nH]c2cc(F)c3nc(-c4ccc(C(=O)NCCN(C)C)c(F)c4)c4c(c3c12)C1CCC4C1 ; Cc1n[nH]c2cc(F)c3nc(-c4ccc(C(=O)NCCN(C)C)nc4)c4c(c3c12)C1CCC4C1 ; Cc1n[nH]c2cc(F)c3nc(-c4ccc(C(=O)NCCN(C)C)cn4)c4c(c3c12)C1CCC4C1 ; Cc1n[nH]c2ccc3nc(-c4ccc(C(=O)NCCN(C)C)cc4)c4c(c3c12)CCCC4]

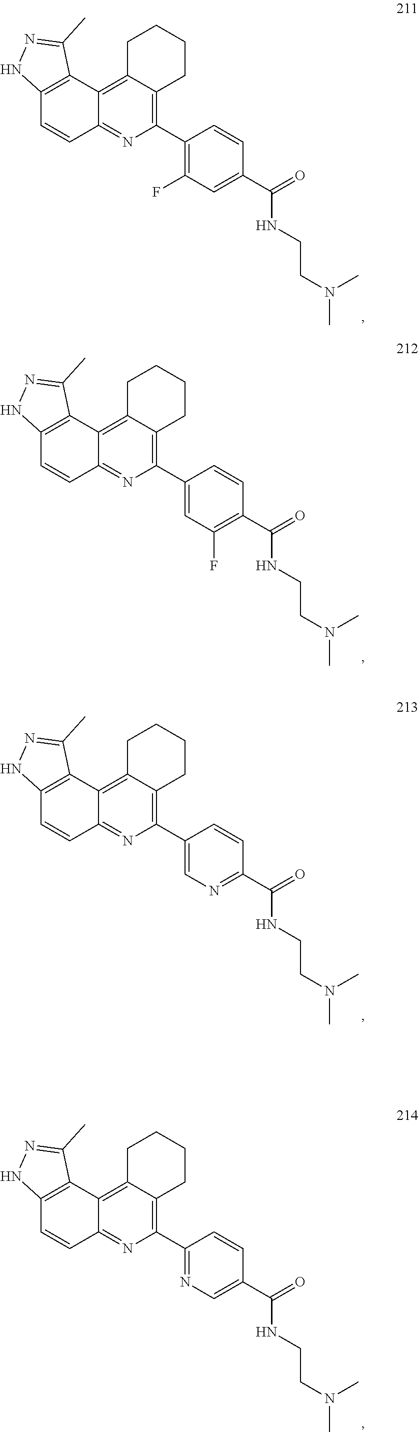 [SMILES: Cc1n[nH]c2ccc3nc(-c4ccc(C(=O)NCCN(C)C)cc4F)c4c(c3c12)CCCC4 ; Cc1n[nH]c2ccc3nc(-c4ccc(C(=O)NCCN(C)C)c(F)c4)c4c(c3c12)CCCC4 ; Cc1n[nH]c2ccc3nc(-c4ccc(C(=O)NCCN(C)C)nc4)c4c(c3c12)CCCC4 ; Cc1n[nH]c2ccc3nc(-c4ccc(C(=O)NCCN(C)C)cn4)c4c(c3c12)CCCC4]

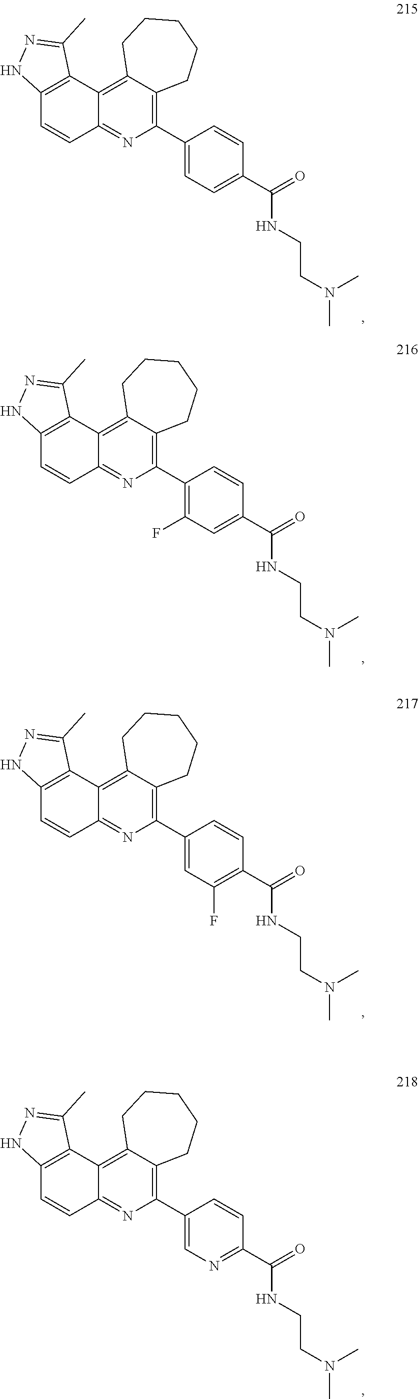 [SMILES: Cc1n[nH]c2ccc3nc(-c4ccc(C(=O)NCCN(C)C)cc4)c4c(c3c12)CCCCC4 ; Cc1n[nH]c2ccc3nc(-c4ccc(C(=O)NCCN(C)C)cc4F)c4c(c3c12)CCCCC4 ; Cc1n[nH]c2ccc3nc(-c4ccc(C(=O)NCCN(C)C)c(F)c4)c4c(c3c12)CCCCC4 ; Cc1n[nH]c2ccc3nc(-c4ccc(C(=O)NCCN(C)C)nc4)c4c(c3c12)CCCCC4]

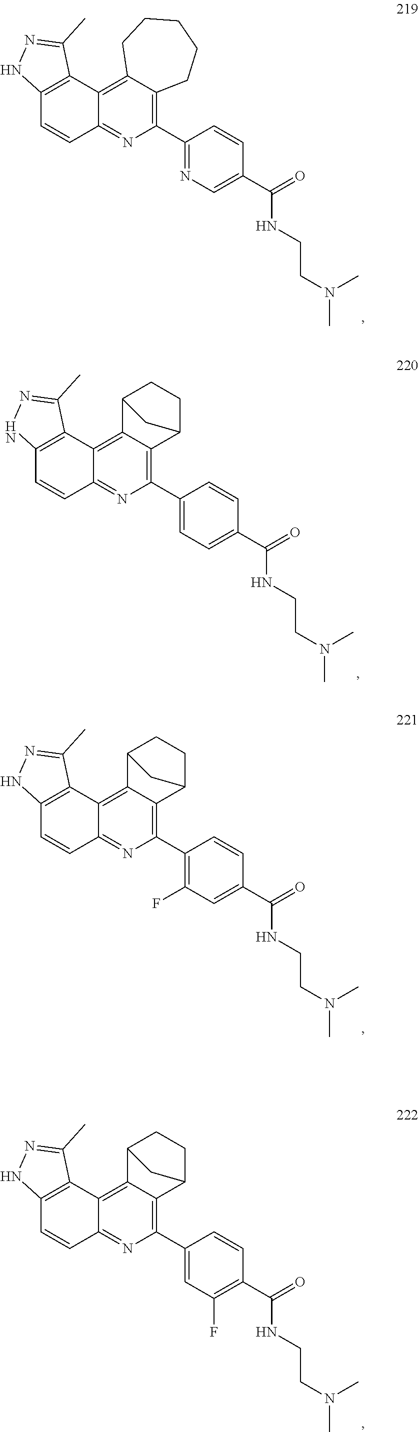 [SMILES: Cc1n[nH]c2ccc3nc(-c4ccc(C(=O)NCCN(C)C)cn4)c4c(c3c12)CCCCC4 ; Cc1n[nH]c2ccc3nc(-c4ccc(C(=O)NCCN(C)C)cc4)c4c(c3c12)C1CCC4C1 ; Cc1n[nH]c2ccc3nc(-c4ccc(C(=O)NCCN(C)C)cc4F)c4c(c3c12)C1CCC4C1 ; Cc1n[nH]c2ccc3nc(-c4ccc(C(=O)NCCN(C)C)c(F)c4)c4c(c3c12)C1CCC4C1]

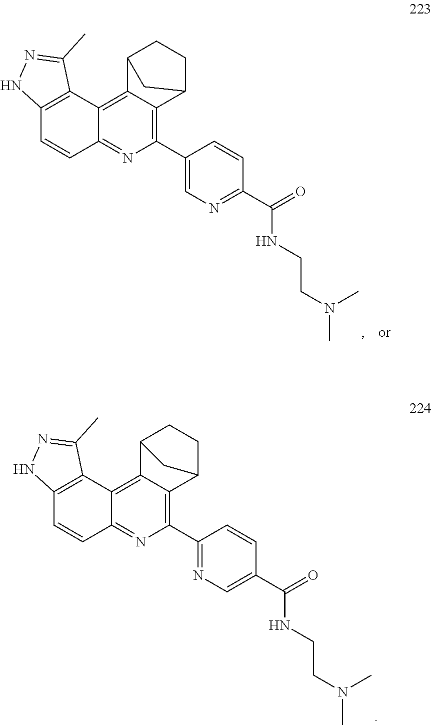 [SMILES: Cc1n[nH]c2ccc3nc(-c4ccc(C(=O)NCCN(C)C)nc4)c4c(c3c12)C1CCC4C1 ; Cc1n[nH]c2ccc3nc(-c4ccc(C(=O)NCCN(C)C)cn4)c4c(c3c12)C1CCC4C1]

24. The compound of claim 1 having a structure of:

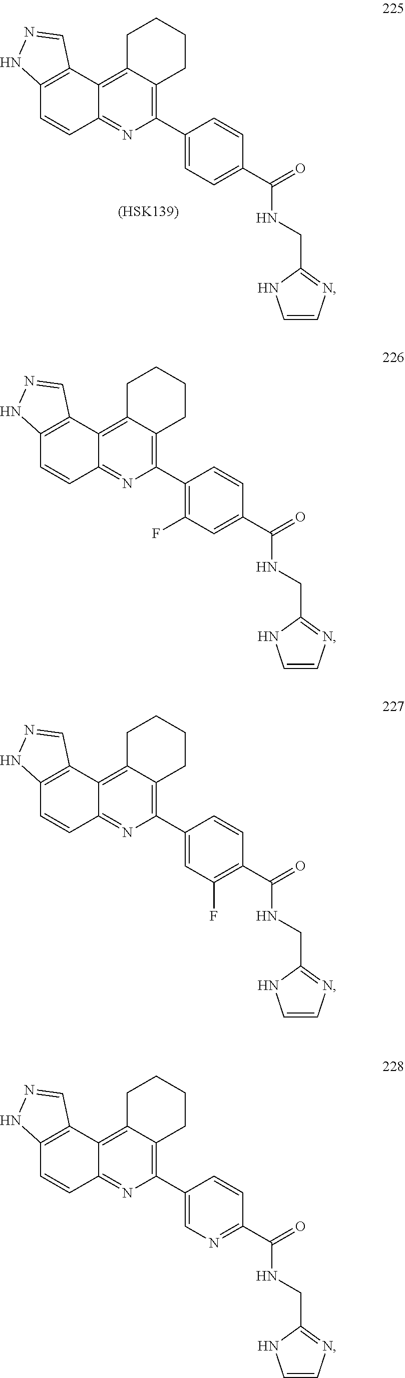 [SMILES: O=C(NCc1ncc[nH]1)c1ccc(-c2nc3ccc4[nH]ncc4c3c3c2CCCC3)c(F)c1 ; O=C(NCc1ncc[nH]1)c1ccc(-c2nc3ccc4[nH]ncc4c3c3c2CCCC3)cc1 ; "(HSK139)" ; O=C(NCc1ncc[nH]1)c1ccc(-c2nc3ccc4[nH]ncc4c3c3c2CCCC3)cc1F ; O=C(NCc1ncc[nH]1)c1ccc(-c2nc3ccc4[nH]ncc4c3c3c2CCCC3)cn1]

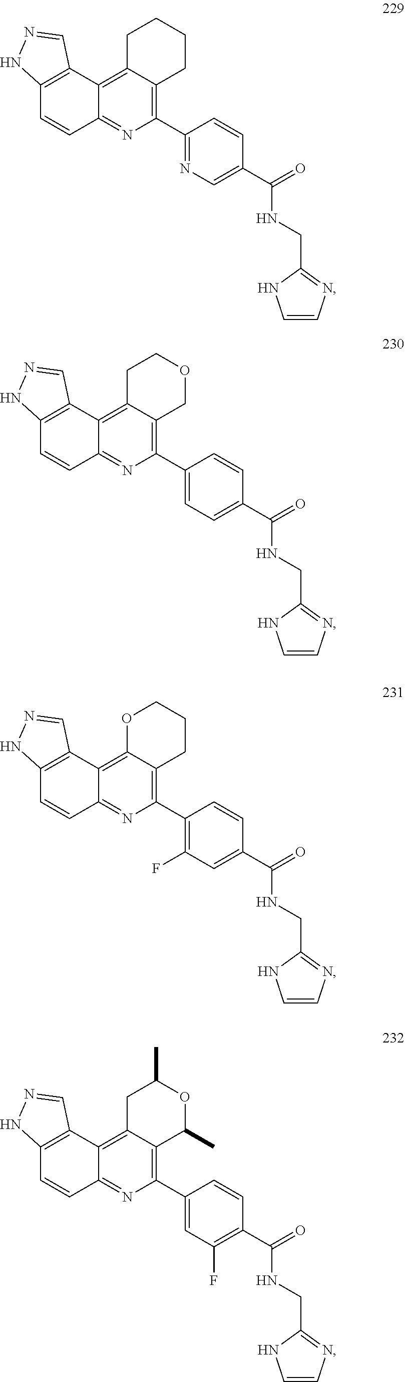 [SMILES: O=C(NCc1ncc[nH]1)c1ccc(-c2nc3ccc4[nH]ncc4c3c3c2CCCC3)nc1 ; O=C(NCc1ncc[nH]1)c1ccc(-c2nc3ccc4[nH]ncc4c3c3c2COCC3)cc1 ; O=C(NCc1ncc[nH]1)c1ccc(-c2nc3ccc4[nH]ncc4c3c3c2CCCO3)c(F)c1 ; C[C@@H]1Cc2c(c(-c3ccc(C(=O)NCc4ncc[nH]4)c(F)c3)nc3ccc4[nH]ncc4c23)[C@H](C)O1]

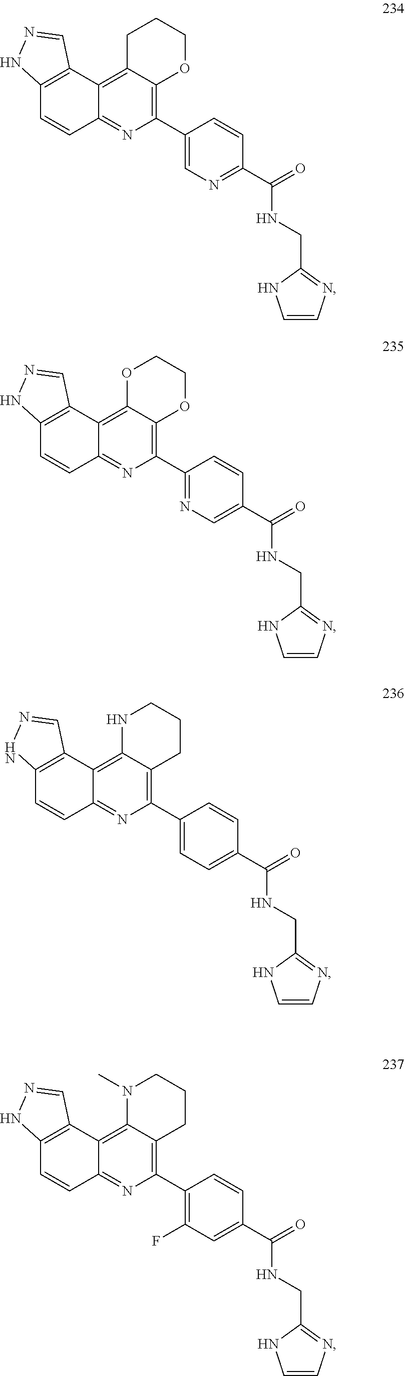 [SMILES: O=C(NCc1ncc[nH]1)c1ccc(-c2nc3ccc4[nH]ncc4c3c3c2OCCC3)cn1 ; O=C(NCc1ncc[nH]1)c1ccc(-c2nc3ccc4[nH]ncc4c3c3c2OCCO3)nc1 ; O=C(NCc1ncc[nH]1)c1ccc(-c2nc3ccc4[nH]ncc4c3c3c2CCCN3)cc1 ; CN1CCCc2c(-c3ccc(C(=O)NCc4ncc[nH]4)cc3F)nc3ccc4[nH]ncc4c3c21]

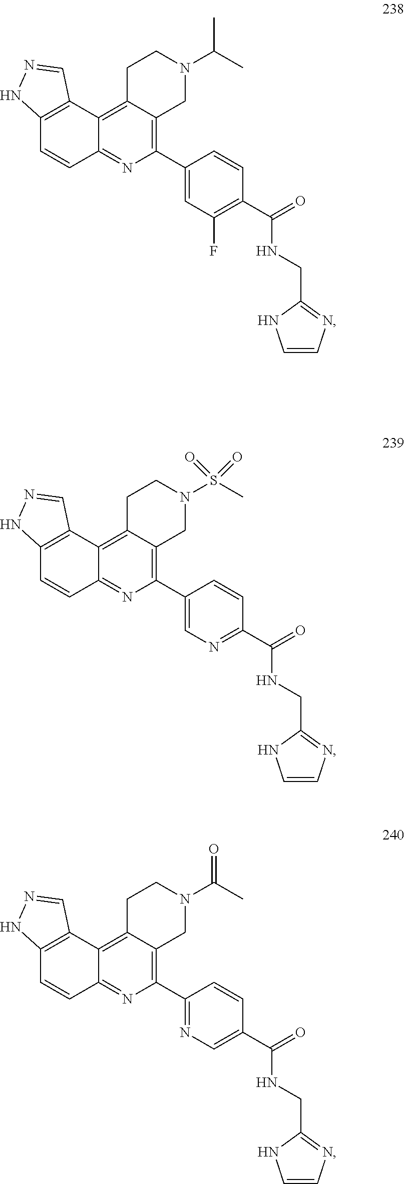 [SMILES: CC(C)N1CCc2c(c(-c3ccc(C(=O)NCc4ncc[nH]4)c(F)c3)nc3ccc4[nH]ncc4c23)C1 ; CS(=O)(=O)N1CCc2c(c(-c3ccc(C(=O)NCc4ncc[nH]4)nc3)nc3ccc4[nH]ncc4c23)C1 ; CC(=O)N1CCc2c(c(-c3ccc(C(=O)NCc4ncc[nH]4)cn3)nc3ccc4[nH]ncc4c23)C1]

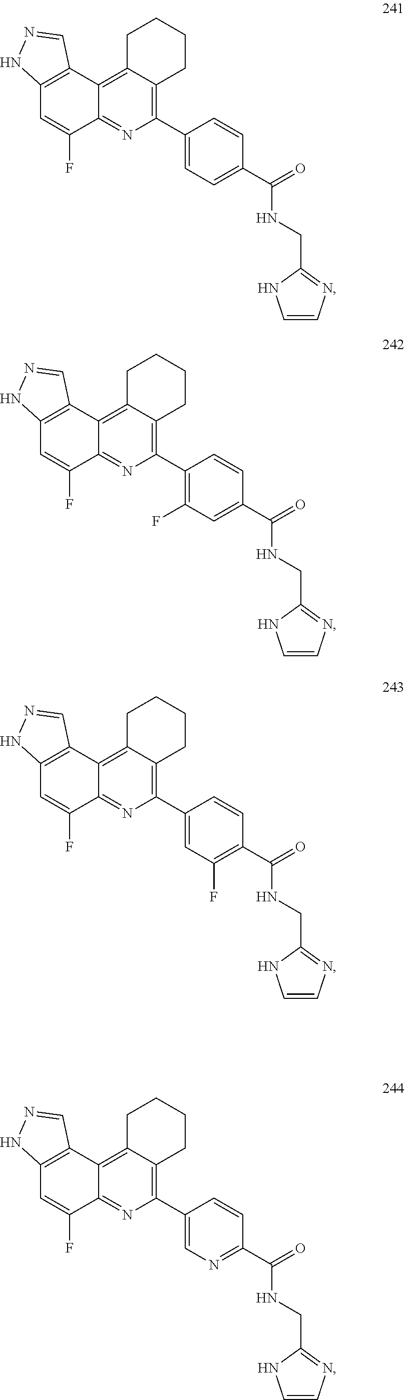 [SMILES: O=C(NCc1ncc[nH]1)c1ccc(-c2nc3c(F)cc4[nH]ncc4c3c3c2CCCC3)cc1 ; O=C(NCc1ncc[nH]1)c1ccc(-c2nc3c(F)cc4[nH]ncc4c3c3c2CCCC3)c(F)c1 ; O=C(NCc1ncc[nH]1)c1ccc(-c2nc3c(F)cc4[nH]ncc4c3c3c2CCCC3)cc1F ; O=C(NCc1ncc[nH]1)c1ccc(-c2nc3c(F)cc4[nH]ncc4c3c3c2CCCC3)cn1]

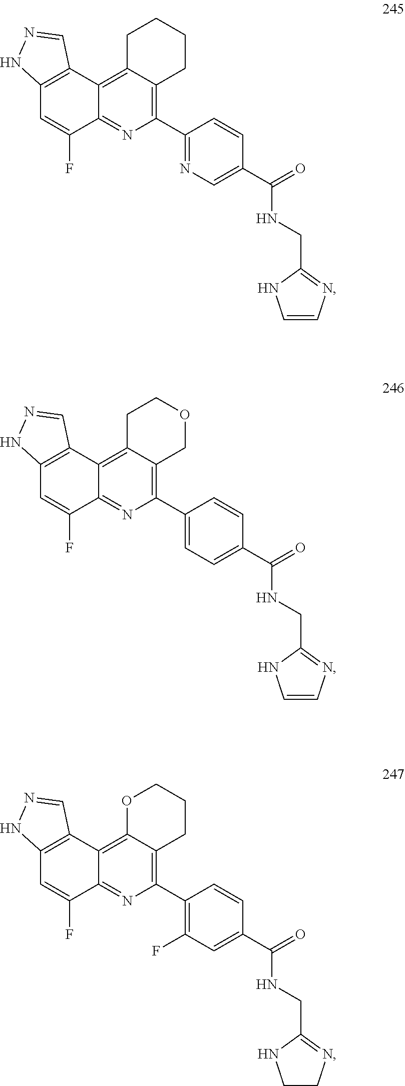 [SMILES: O=C(NCc1ncc[nH]1)c1ccc(-c2nc3c(F)cc4[nH]ncc4c3c3c2CCCC3)nc1 ; O=C(NCc1ncc[nH]1)c1ccc(-c2nc3c(F)cc4[nH]ncc4c3c3c2COCC3)cc1 ; O=C(NCC1=NCCN1)c1ccc(-c2nc3c(F)cc4[nH]ncc4c3c3c2CCCO3)c(F)c1]

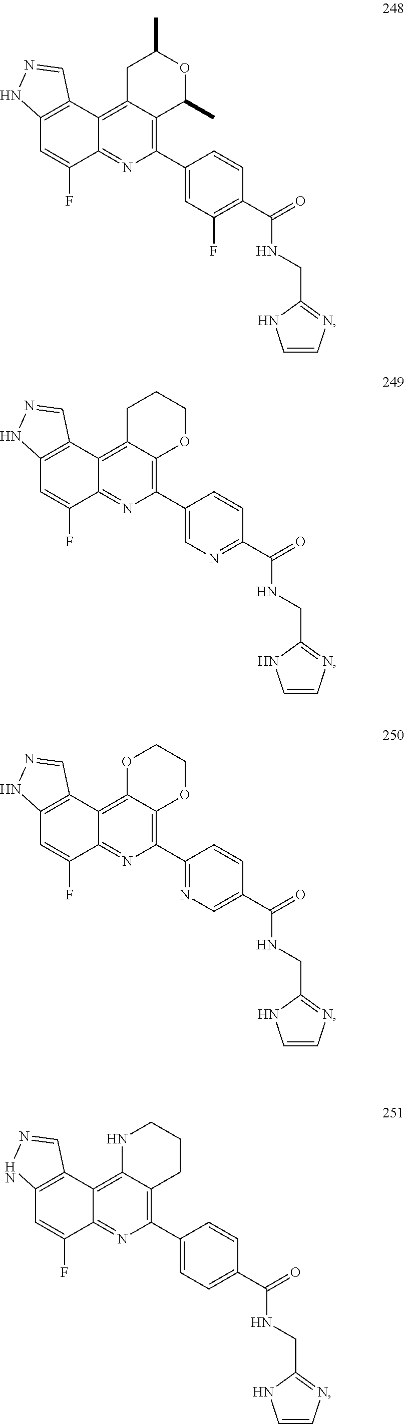 [SMILES: C[C@@H]1Cc2c(c(-c3ccc(C(=O)NCc4ncc[nH]4)c(F)c3)nc3c(F)cc4[nH]ncc4c23)[C@H](C)O1 ; O=C(NCc1ncc[nH]1)c1ccc(-c2nc3c(F)cc4[nH]ncc4c3c3c2OCCC3)cn1 ; O=C(NCc1ncc[nH]1)c1ccc(-c2nc3c(F)cc4[nH]ncc4c3c3c2OCCO3)nc1 ; O=C(NCc1ncc[nH]1)c1ccc(-c2nc3c(F)cc4[nH]ncc4c3c3c2CCCN3)cc1]

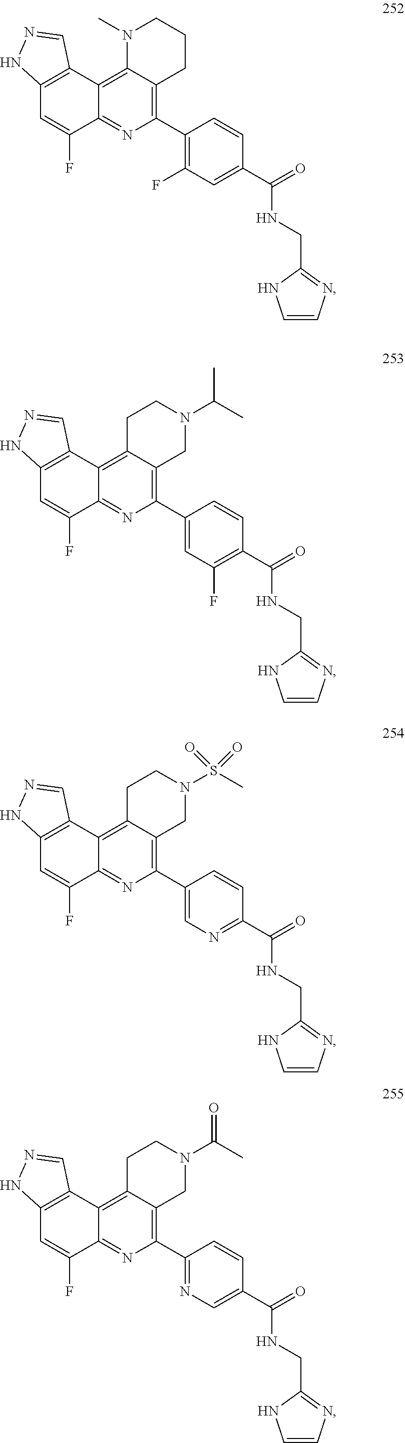 [SMILES: CN1CCCc2c(-c3ccc(C(=O)NCc4ncc[nH]4)cc3F)nc3c(F)cc4[nH]ncc4c3c21 ; CC(C)N1CCc2c(c(-c3ccc(C(=O)NCc4ncc[nH]4)c(F)c3)nc3c(F)cc4[nH]ncc4c23)C1 ; CS(=O)(=O)N1CCc2c(c(-c3ccc(C(=O)NCc4ncc[nH]4)nc3)nc3c(F)cc4[nH]ncc4c23)C1 ; CC(=O)N1CCc2c(c(-c3ccc(C(=O)NCc4ncc[nH]4)cn3)nc3c(F)cc4[nH]ncc4c23)C1]

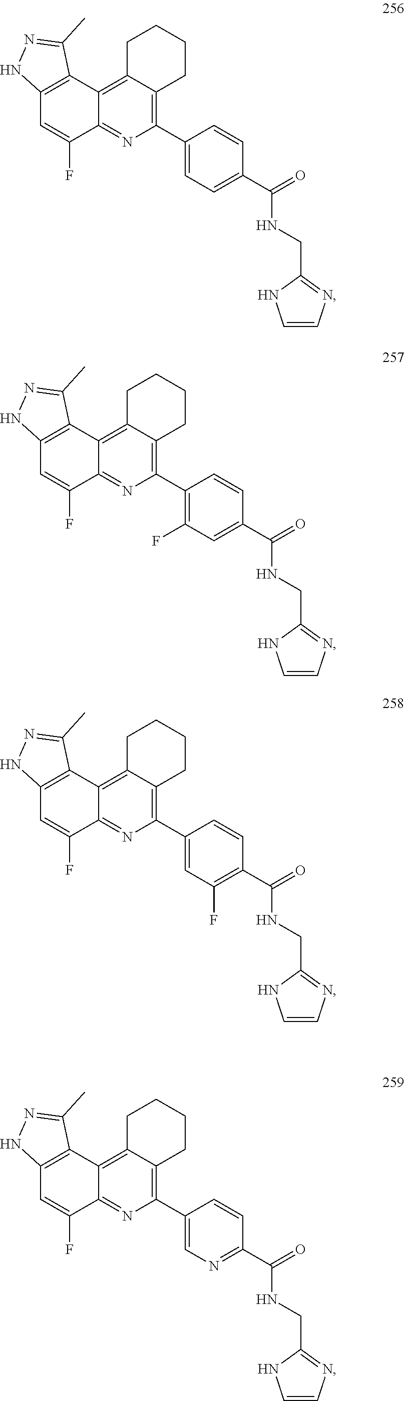 [SMILES: Cc1n[nH]c2cc(F)c3nc(-c4ccc(C(=O)NCc5ncc[nH]5)cc4)c4c(c3c12)CCCC4 ; Cc1n[nH]c2cc(F)c3nc(-c4ccc(C(=O)NCc5ncc[nH]5)cc4F)c4c(c3c12)CCCC4 ; Cc1n[nH]c2cc(F)c3nc(-c4ccc(C(=O)NCc5ncc[nH]5)c(F)c4)c4c(c3c12)CCCC4 ; Cc1n[nH]c2cc(F)c3nc(-c4ccc(C(=O)NCc5ncc[nH]5)nc4)c4c(c3c12)CCCC4]

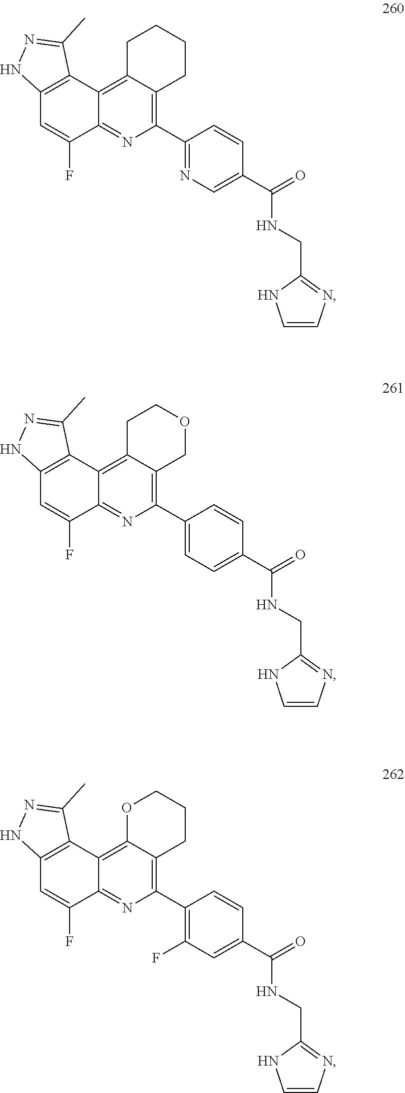 [SMILES: Cc1n[nH]c2cc(F)c3nc(-c4ccc(C(=O)NCc5ncc[nH]5)cn4)c4c(c3c12)CCCC4 ; Cc1n[nH]c2cc(F)c3nc(-c4ccc(C(=O)NCc5ncc[nH]5)cc4)c4c(c3c12)CCOC4 ; Cc1n[nH]c2cc(F)c3nc(-c4ccc(C(=O)NCc5ncc[nH]5)cc4F)c4c(c3c12)OCCC4]

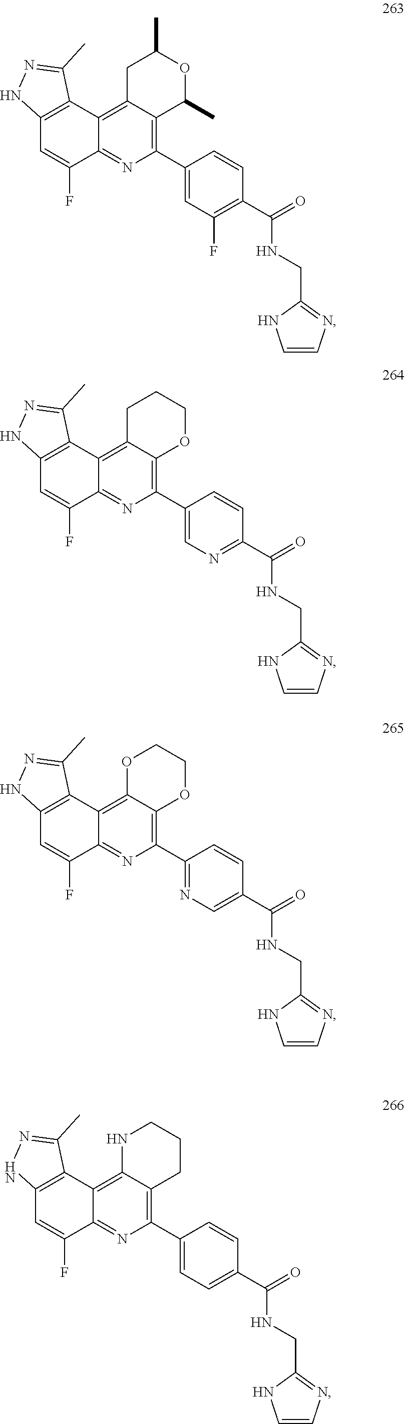 [SMILES: Cc1n[nH]c2cc(F)c3nc(-c4ccc(C(=O)NCc5ncc[nH]5)c(F)c4)c4c(c3c12)C[C@@H](C)O[C@H]4C ; Cc1n[nH]c2cc(F)c3nc(-c4ccc(C(=O)NCc5ncc[nH]5)nc4)c4c(c3c12)CCCO4 ; Cc1n[nH]c2cc(F)c3nc(-c4ccc(C(=O)NCc5ncc[nH]5)cn4)c4c(c3c12)OCCO4 ; Cc1n[nH]c2cc(F)c3nc(-c4ccc(C(=O)NCc5ncc[nH]5)cc4)c4c(c3c12)NCCC4]

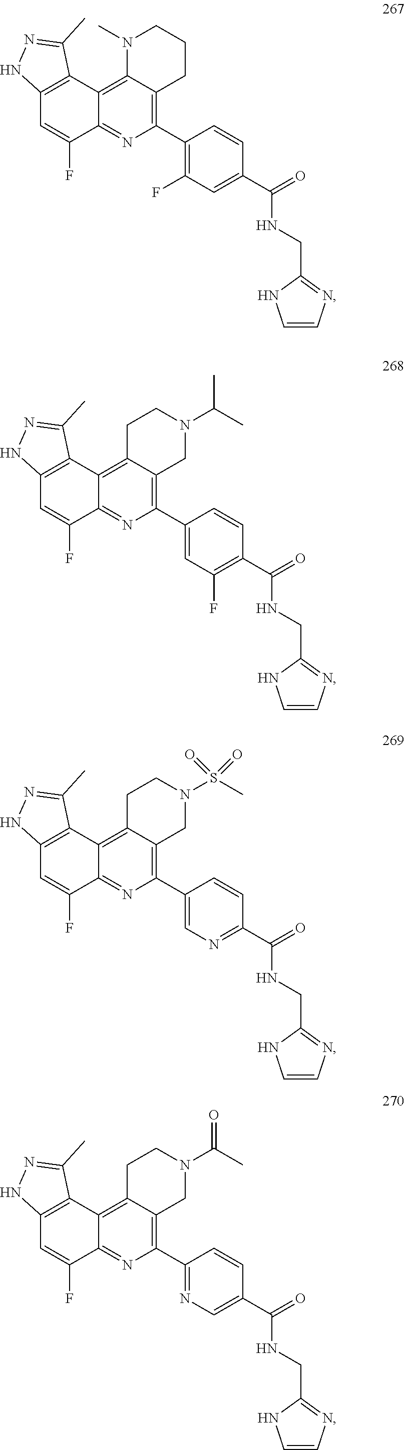 [SMILES: Cc1n[nH]c2cc(F)c3nc(-c4ccc(C(=O)NCc5ncc[nH]5)cc4F)c4c(c3c12)N(C)CCC4 ; Cc1n[nH]c2cc(F)c3nc(-c4ccc(C(=O)NCc5ncc[nH]5)c(F)c4)c4c(c3c12)CCN(C(C)C)C4 ; Cc1n[nH]c2cc(F)c3nc(-c4ccc(C(=O)NCc5ncc[nH]5)nc4)c4c(c3c12)CCN(S(C)(=O)=O)C4 ; CC(=O)N1CCc2c(c(-c3ccc(C(=O)NCc4ncc[nH]4)cn3)nc3c(F)cc4[nH]nc(C)c4c23)C1]

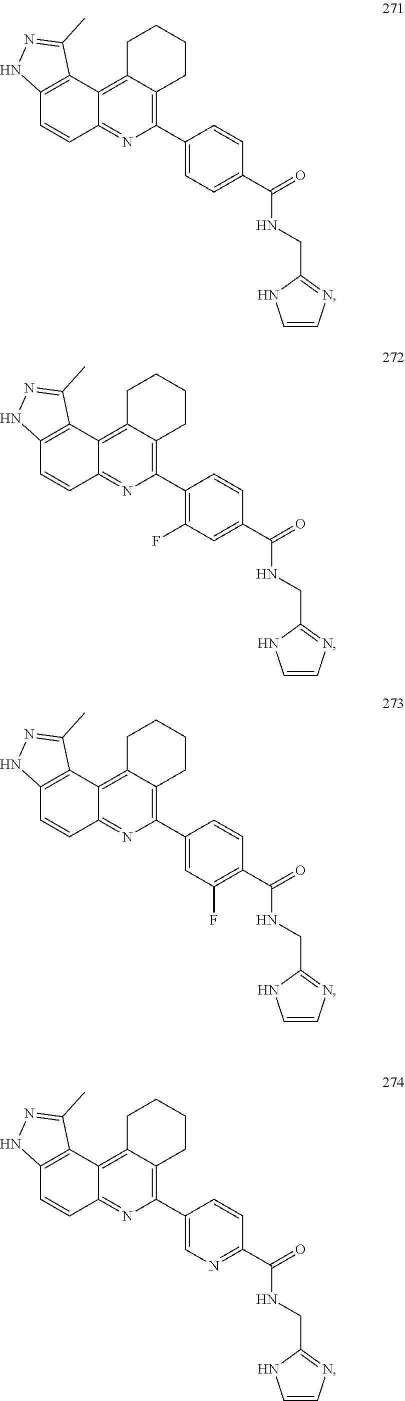 [SMILES: Cc1n[nH]c2ccc3nc(-c4ccc(C(=O)NCc5ncc[nH]5)cc4)c4c(c3c12)CCCC4 ; Cc1n[nH]c2ccc3nc(-c4ccc(C(=O)NCc5ncc[nH]5)cc4F)c4c(c3c12)CCCC4 ; Cc1n[nH]c2ccc3nc(-c4ccc(C(=O)NCc5ncc[nH]5)c(F)c4)c4c(c3c12)CCCC4 ; Cc1n[nH]c2ccc3nc(-c4ccc(C(=O)NCc5ncc[nH]5)nc4)c4c(c3c12)CCCC4]

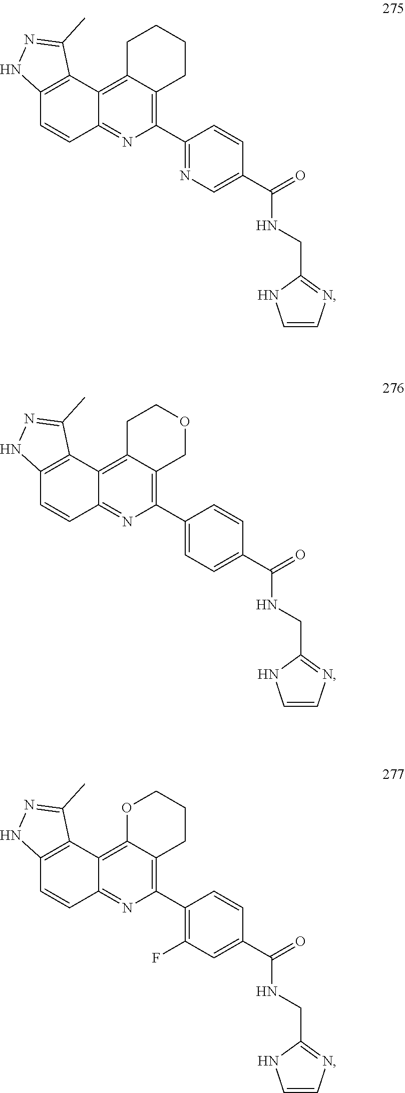 [SMILES: Cc1n[nH]c2ccc3nc(-c4ccc(C(=O)NCc5ncc[nH]5)cn4)c4c(c3c12)CCCC4 ; Cc1n[nH]c2ccc3nc(-c4ccc(C(=O)NCc5ncc[nH]5)cc4)c4c(c3c12)CCOC4 ; Cc1n[nH]c2ccc3nc(-c4ccc(C(=O)NCc5ncc[nH]5)cc4F)c4c(c3c12)OCCC4]

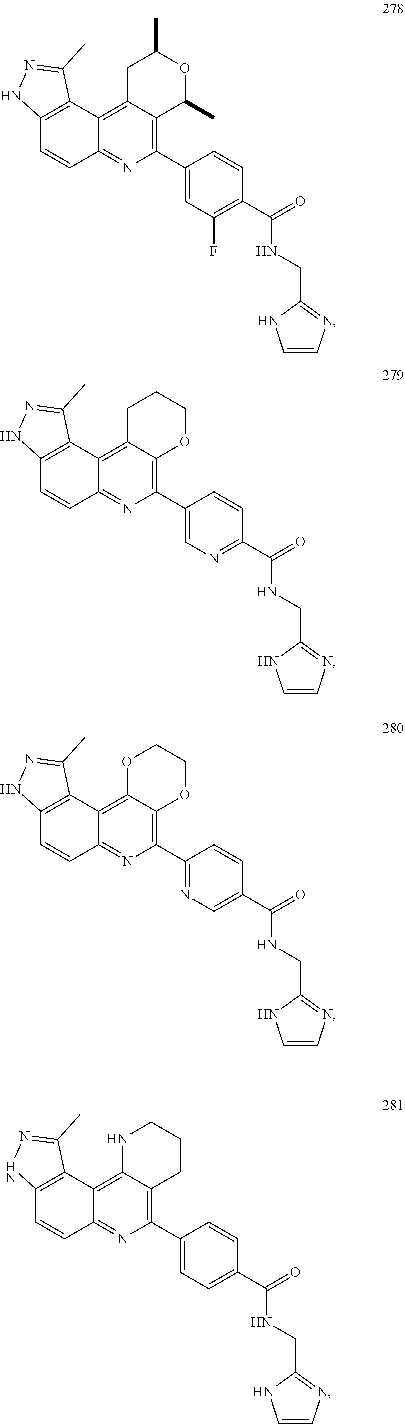 [SMILES: Cc1n[nH]c2ccc3nc(-c4ccc(C(=O)NCc5ncc[nH]5)c(F)c4)c4c(c3c12)C[C@@H](C)O[C@H]4C ; Cc1n[nH]c2ccc3nc(-c4ccc(C(=O)NCc5ncc[nH]5)nc4)c4c(c3c12)CCCO4 ; Cc1n[nH]c2ccc3nc(-c4ccc(C(=O)NCc5ncc[nH]5)cn4)c4c(c3c12)OCCO4 ; Cc1n[nH]c2ccc3nc(-c4ccc(C(=O)NCc5ncc[nH]5)cc4)c4c(c3c12)NCCC4]

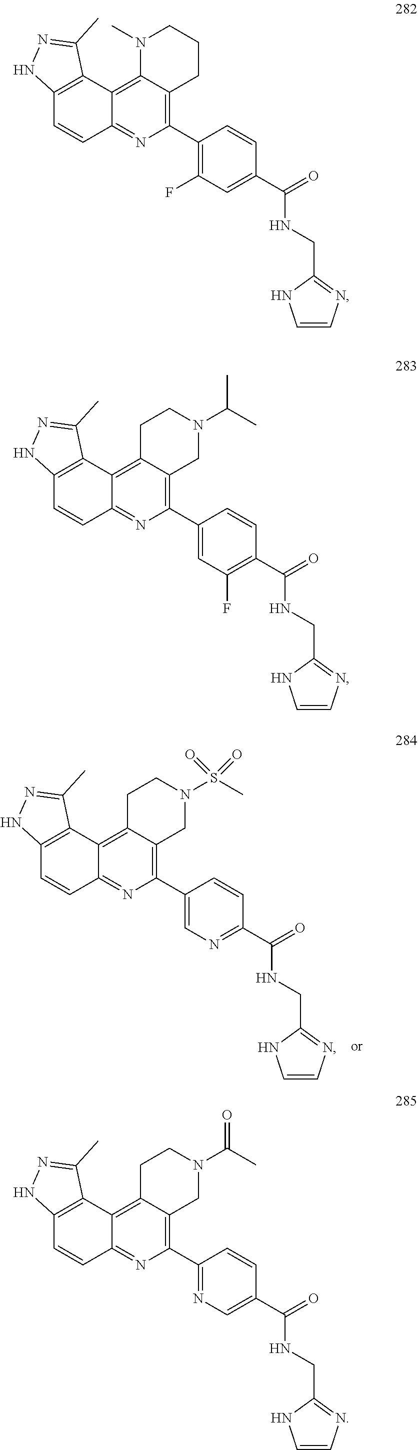 [SMILES: Cc1n[nH]c2ccc3nc(-c4ccc(C(=O)NCc5ncc[nH]5)cc4F)c4c(c3c12)N(C)CCC4 ; Cc1n[nH]c2ccc3nc(-c4ccc(C(=O)NCc5ncc[nH]5)c(F)c4)c4c(c3c12)CCN(C(C)C)C4 ; Cc1n[nH]c2ccc3nc(-c4ccc(C(=O)NCc5ncc[nH]5)nc4)c4c(c3c12)CCN(S(C)(=O)=O)C4 ; "or" ; CC(=O)N1CCc2c(c(-c3ccc(C(=O)NCc4ncc[nH]4)cn3)nc3ccc4[nH]nc(C)c4c23)C1]

25. The compound of claim 1 having a structure of:

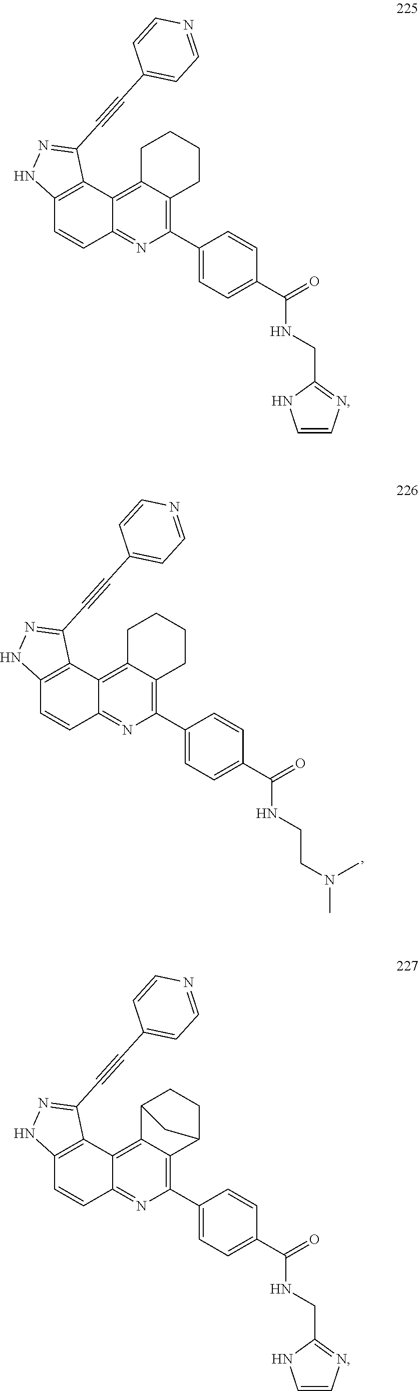 [SMILES: O=C(NCc1ncc[nH]1)c1ccc(-c2nc3ccc4[nH]nc(C#Cc5ccncc5)c4c3c3c2CCCC3)cc1 ; CN(C)CCNC(=O)c1ccc(-c2nc3ccc4[nH]nc(C#Cc5ccncc5)c4c3c3c2CCCC3)cc1 ; O=C(NCc1ncc[nH]1)c1ccc(-c2nc3ccc4[nH]nc(C#Cc5ccncc5)c4c3c3c2C2CCC3C2)cc1]

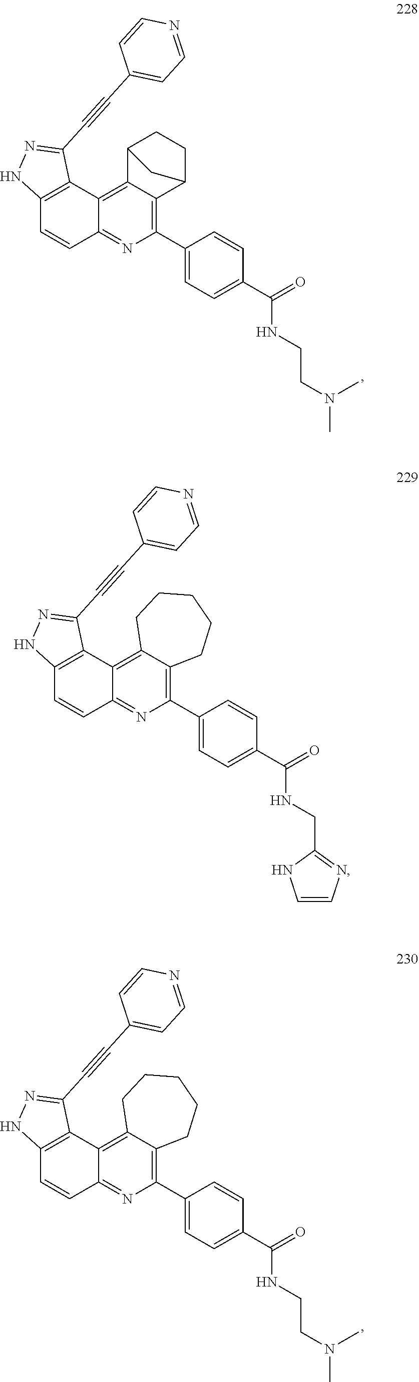 [SMILES: CN(C)CCNC(=O)c1ccc(-c2nc3ccc4[nH]nc(C#Cc5ccncc5)c4c3c3c2C2CCC3C2)cc1 ; O=C(NCc1ncc[nH]1)c1ccc(-c2nc3ccc4[nH]nc(C#Cc5ccncc5)c4c3c3c2CCCCC3)cc1 ; CN(C)CCNC(=O)c1ccc(-c2nc3ccc4[nH]nc(C#Cc5ccncc5)c4c3c3c2CCCCC3)cc1]

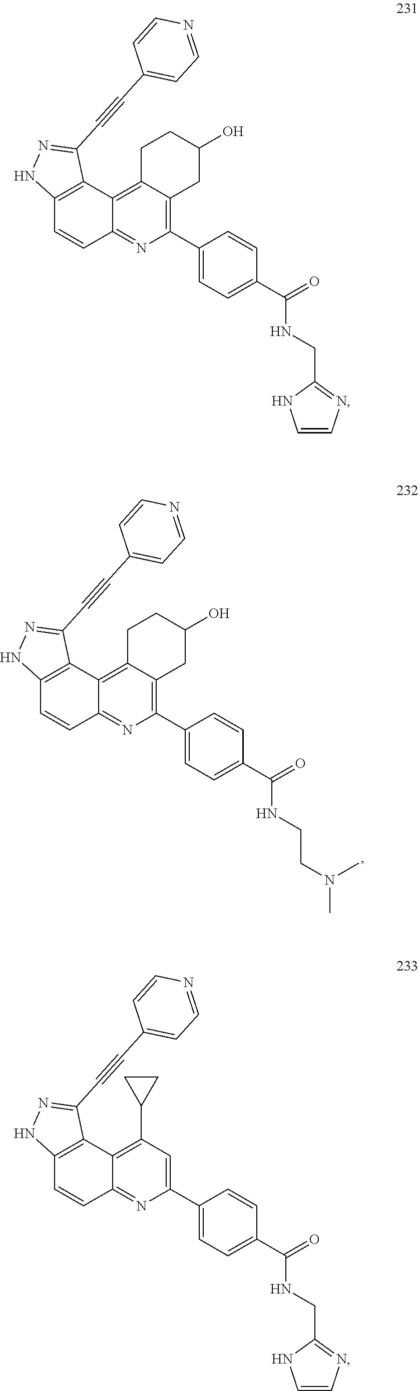 [SMILES: O=C(NCc1ncc[nH]1)c1ccc(-c2nc3ccc4[nH]nc(C#Cc5ccncc5)c4c3c3c2CC(O)CC3)cc1 ; CN(C)CCNC(=O)c1ccc(-c2nc3ccc4[nH]nc(C#Cc5ccncc5)c4c3c3c2CC(O)CC3)cc1 ; O=C(NCc1ncc[nH]1)c1ccc(-c2cc(C3CC3)c3c(ccc4[nH]nc(C#Cc5ccncc5)c43)n2)cc1]

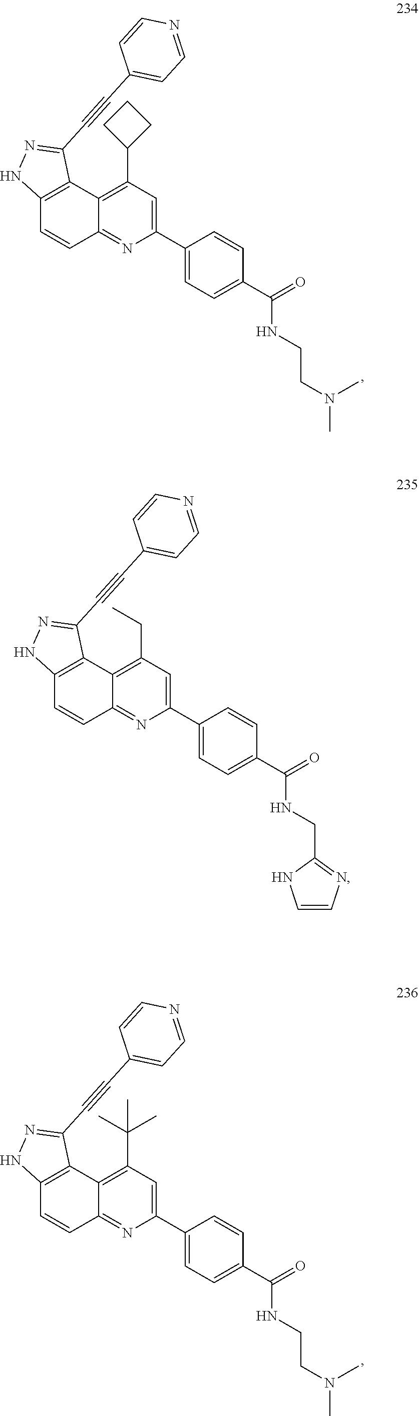 [SMILES: CN(C)CCNC(=O)c1ccc(-c2cc(C3CCC3)c3c(ccc4[nH]nc(C#Cc5ccncc5)c43)n2)cc1 ; CCc1cc(-c2ccc(C(=O)NCc3ncc[nH]3)cc2)nc2ccc3[nH]nc(C#Cc4ccncc4)c3c12 ; CN(C)CCNC(=O)c1ccc(-c2cc(C(C)(C)C)c3c(ccc4[nH]nc(C#Cc5ccncc5)c43)n2)cc1]

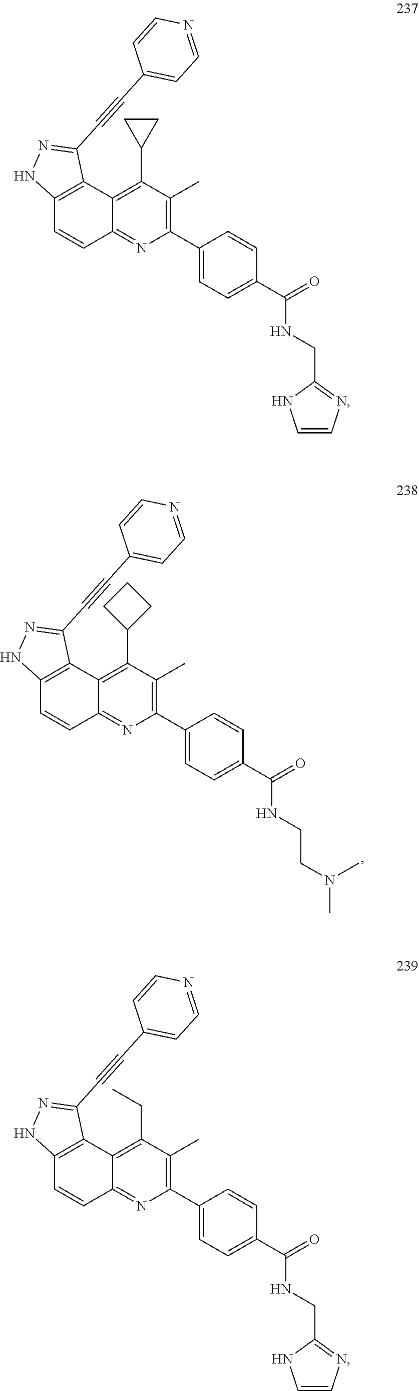 [SMILES: Cc1c(-c2ccc(C(=O)NCc3ncc[nH]3)cc2)nc2ccc3[nH]nc(C#Cc4ccncc4)c3c2c1C1CC1 ; Cc1c(-c2ccc(C(=O)NCCN(C)C)cc2)nc2ccc3[nH]nc(C#Cc4ccncc4)c3c2c1C1CCC1 ; CCc1c(C)c(-c2ccc(C(=O)NCc3ncc[nH]3)cc2)nc2ccc3[nH]nc(C#Cc4ccncc4)c3c12]

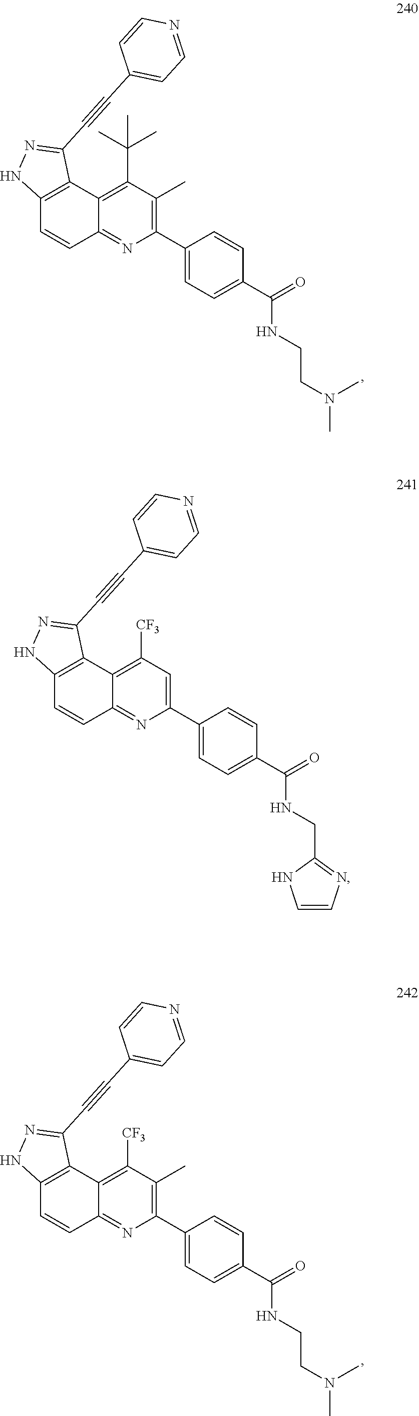 [SMILES: Cc1c(-c2ccc(C(=O)NCCN(C)C)cc2)nc2ccc3[nH]nc(C#Cc4ccncc4)c3c2c1C(C)(C)C ; O=C(NCc1ncc[nH]1)c1ccc(-c2cc(C(F)(F)F)c3c(ccc4[nH]nc(C#Cc5ccncc5)c43)n2)cc1 ; Cc1c(-c2ccc(C(=O)NCCN(C)C)cc2)nc2ccc3[nH]nc(C#Cc4ccncc4)c3c2c1C(F)(F)F]

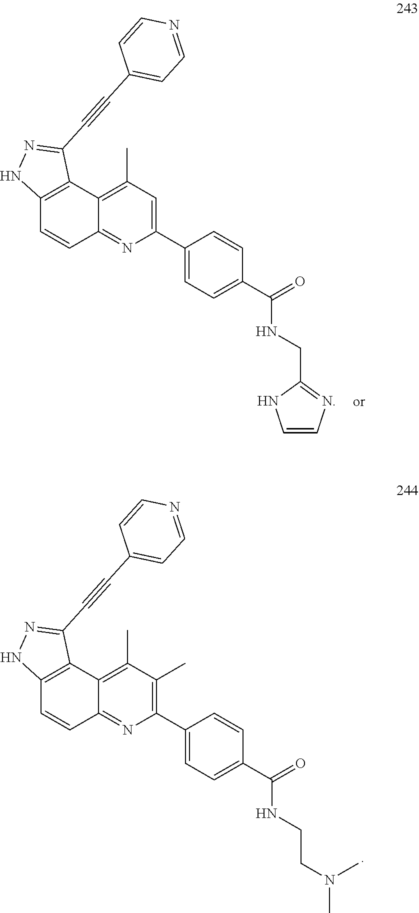 [SMILES: Cc1cc(-c2ccc(C(=O)NCc3ncc[nH]3)cc2)nc2ccc3[nH]nc(C#Cc4ccncc4)c3c12 ; Cc1c(-c2ccc(C(=O)NCCN(C)C)cc2)nc2ccc3[nH]nc(C#Cc4ccncc4)c3c2c1C]

26. The compound of claim 3 having a structure of:

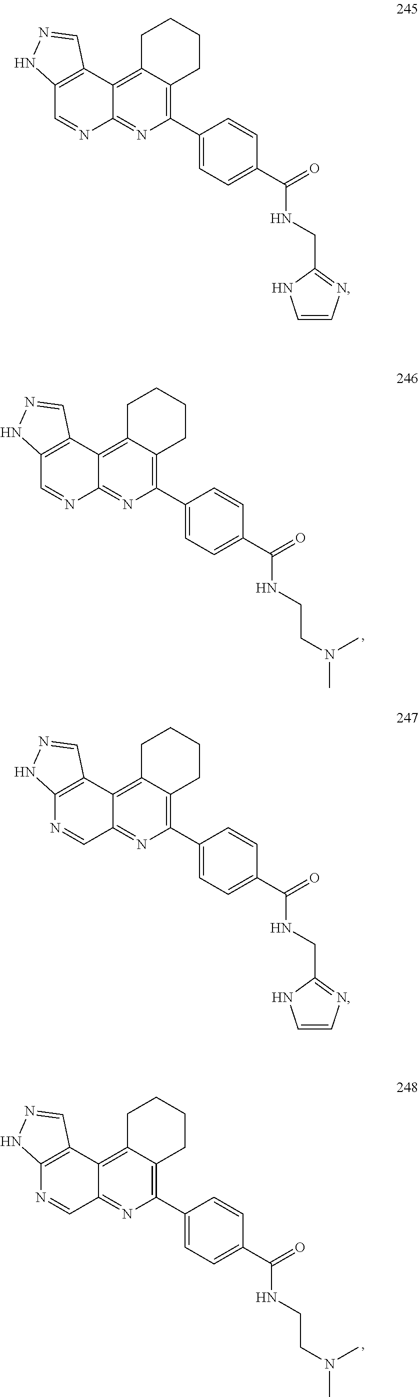 [SMILES: O=C(NCc1ncc[nH]1)c1ccc(-c2nc3ncc4[nH]ncc4c3c3c2CCCC3)cc1 ; CN(C)CCNC(=O)c1ccc(-c2nc3ncc4[nH]ncc4c3c3c2CCCC3)cc1 ; O=C(NCc1ncc[nH]1)c1ccc(-c2nc3cnc4[nH]ncc4c3c3c2CCCC3)cc1 ; CN(C)CCNC(=O)c1ccc(-c2nc3cnc4[nH]ncc4c3c3c2CCCC3)cc1]

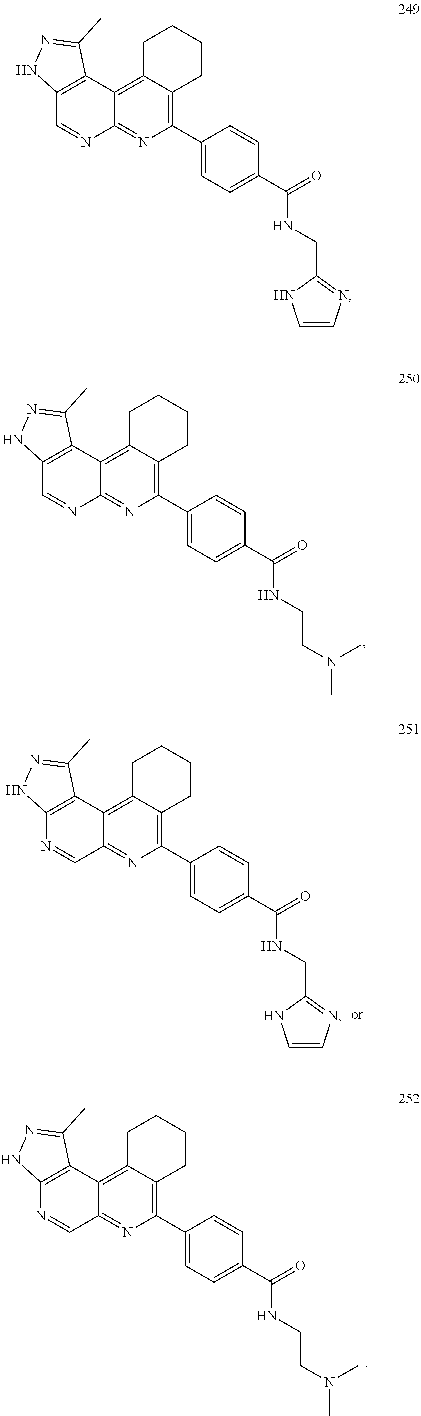 [SMILES: Cc1n[nH]c2cnc3nc(-c4ccc(C(=O)NCc5ncc[nH]5)cc4)c4c(c3c12)CCCC4 ; Cc1n[nH]c2cnc3nc(-c4ccc(C(=O)NCCN(C)C)cc4)c4c(c3c12)CCCC4 ; Cc1n[nH]c2ncc3nc(-c4ccc(C(=O)NCc5ncc[nH]5)cc4)c4c(c3c12)CCCC4 ; "or" ; Cc1n[nH]c2ncc3nc(-c4ccc(C(=O)NCCN(C)C)cc4)c4c(c3c12)CCCC4]

27. The compound of claim 3 having a structure of:

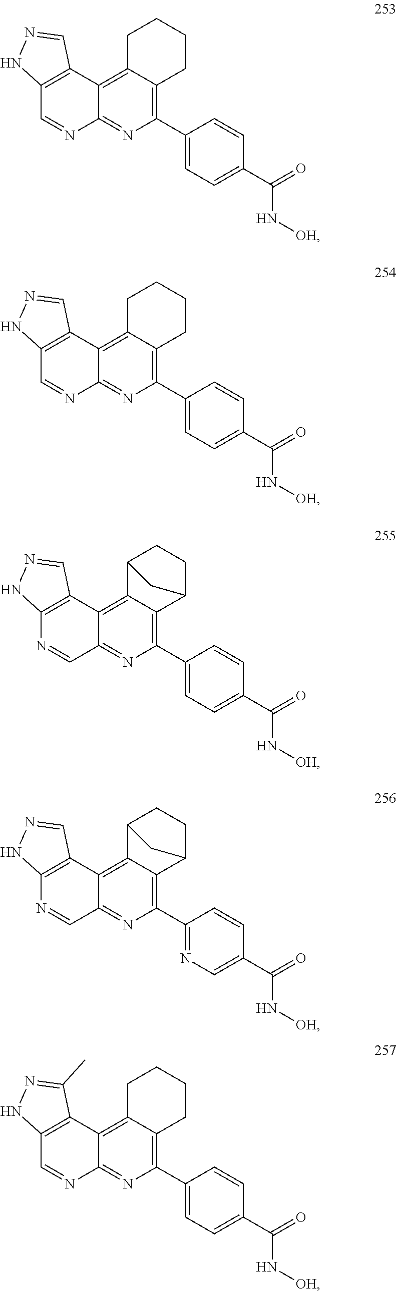 [SMILES: O=C(NO)c1ccc(-c2nc3ncc4[nH]ncc4c3c3c2CCCC3)cc1 ; O=C(NO)c1ccc(-c2nc3ncc4[nH]ncc4c3c3c2CCCC3)cc1 ; O=C(NO)c1ccc(-c2nc3cnc4[nH]ncc4c3c3c2C2CCC3C2)cc1 ; O=C(NO)c1ccc(-c2nc3cnc4[nH]ncc4c3c3c2C2CCC3C2)nc1 ; Cc1n[nH]c2cnc3nc(-c4ccc(C(=O)NO)cc4)c4c(c3c12)CCCC4]

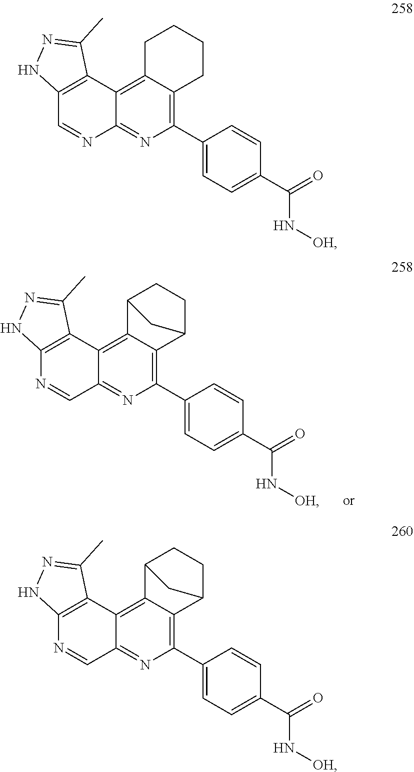 [SMILES: Cc1n[nH]c2cnc3nc(-c4ccc(C(=O)NO)cc4)c4c(c3c12)CCCC4 ; Cc1n[nH]c2ncc3nc(-c4ccc(C(=O)NO)cc4)c4c(c3c12)C1CCC4C1 ; Cc1n[nH]c2ncc3nc(-c4ccc(C(=O)NO)cc4)c4c(c3c12)C1CCC4C1]

28. The compound of claim 3 having a structure of:

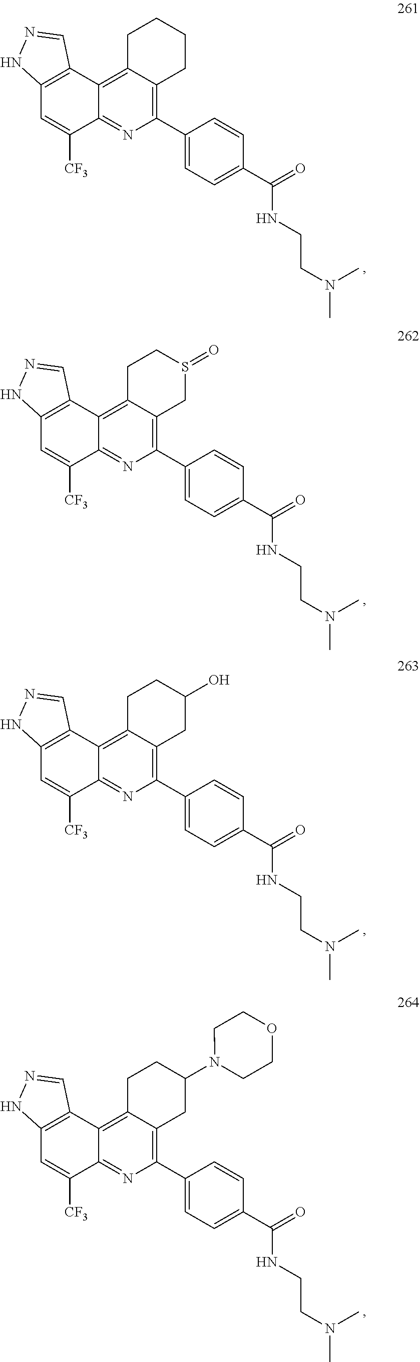 [SMILES: CN(C)CCNC(=O)c1ccc(-c2nc3c(C(F)(F)F)cc4[nH]ncc4c3c3c2CCCC3)cc1 ; CN(C)CCNC(=O)c1ccc(-c2nc3c(C(F)(F)F)cc4[nH]ncc4c3c3c2CS(=O)CC3)cc1 ; CN(C)CCNC(=O)c1ccc(-c2nc3c(C(F)(F)F)cc4[nH]ncc4c3c3c2CC(O)CC3)cc1 ; CN(C)CCNC(=O)c1ccc(-c2nc3c(C(F)(F)F)cc4[nH]ncc4c3c3c2CC(N2CCOCC2)CC3)cc1]

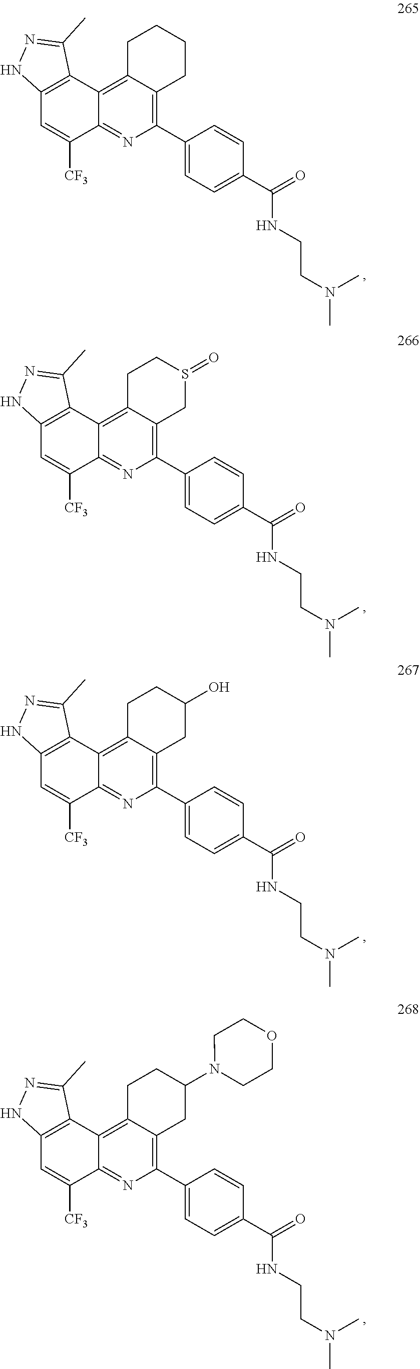 [SMILES: Cc1n[nH]c2cc(C(F)(F)F)c3nc(-c4ccc(C(=O)NCCN(C)C)cc4)c4c(c3c12)CCCC4 ; Cc1n[nH]c2cc(C(F)(F)F)c3nc(-c4ccc(C(=O)NCCN(C)C)cc4)c4c(c3c12)CCS(=O)C4 ; Cc1n[nH]c2cc(C(F)(F)F)c3nc(-c4ccc(C(=O)NCCN(C)C)cc4)c4c(c3c12)CCC(O)C4 ; Cc1n[nH]c2cc(C(F)(F)F)c3nc(-c4ccc(C(=O)NCCN(C)C)cc4)c4c(c3c12)CCC(N1CCOCC1)C4]

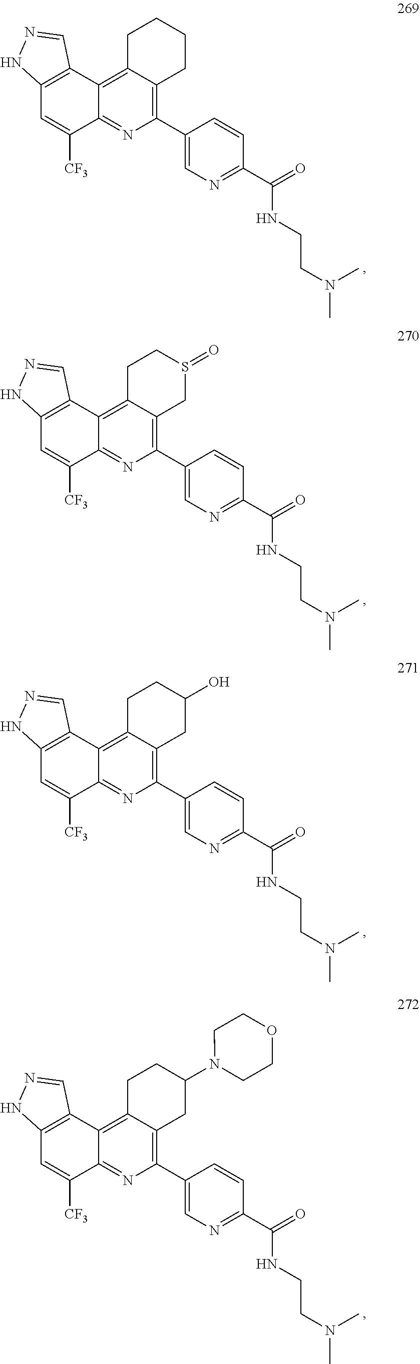 [SMILES: CN(C)CCNC(=O)c1ccc(-c2nc3c(C(F)(F)F)cc4[nH]ncc4c3c3c2CCCC3)cn1 ; CN(C)CCNC(=O)c1ccc(-c2nc3c(C(F)(F)F)cc4[nH]ncc4c3c3c2CS(=O)CC3)cn1 ; CN(C)CCNC(=O)c1ccc(-c2nc3c(C(F)(F)F)cc4[nH]ncc4c3c3c2CC(O)CC3)cn1 ; CN(C)CCNC(=O)c1ccc(-c2nc3c(C(F)(F)F)cc4[nH]ncc4c3c3c2CC(N2CCOCC2)CC3)cn1]

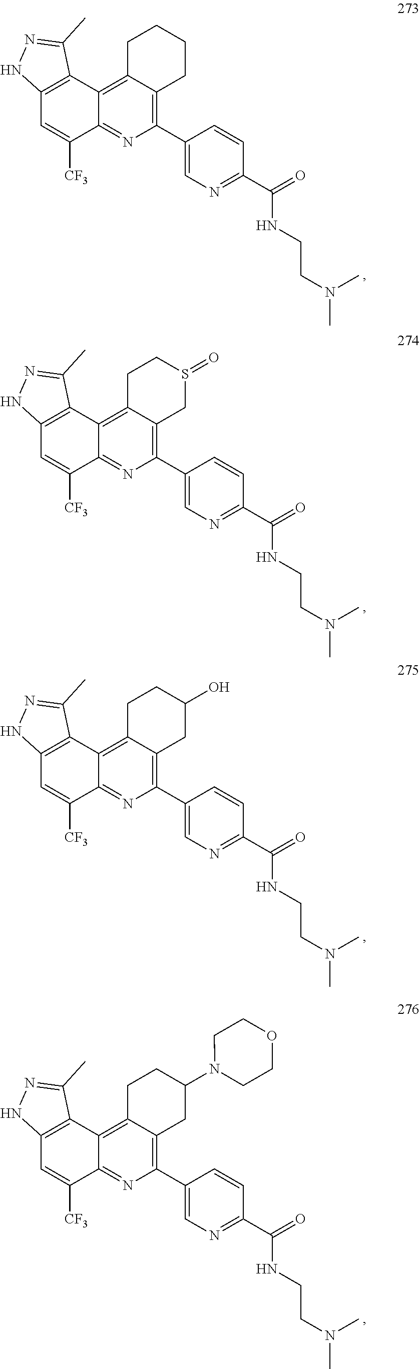 [SMILES: Cc1n[nH]c2cc(C(F)(F)F)c3nc(-c4ccc(C(=O)NCCN(C)C)nc4)c4c(c3c12)CCCC4 ; Cc1n[nH]c2cc(C(F)(F)F)c3nc(-c4ccc(C(=O)NCCN(C)C)nc4)c4c(c3c12)CCS(=O)C4 ; Cc1n[nH]c2cc(C(F)(F)F)c3nc(-c4ccc(C(=O)NCCN(C)C)nc4)c4c(c3c12)CCC(O)C4 ; Cc1n[nH]c2cc(C(F)(F)F)c3nc(-c4ccc(C(=O)NCCN(C)C)nc4)c4c(c3c12)CCC(N1CCOCC1)C4]

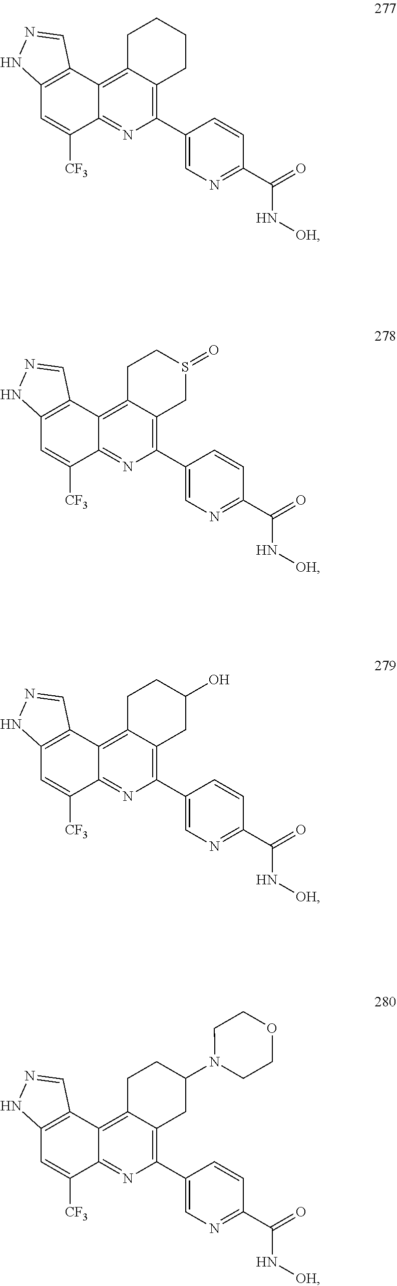 [SMILES: O=C(NO)c1ccc(-c2nc3c(C(F)(F)F)cc4[nH]ncc4c3c3c2CCCC3)cn1 ; O=C(NO)c1ccc(-c2nc3c(C(F)(F)F)cc4[nH]ncc4c3c3c2CS(=O)CC3)cn1 ; O=C(NO)c1ccc(-c2nc3c(C(F)(F)F)cc4[nH]ncc4c3c3c2CC(O)CC3)cn1 ; O=C(NO)c1ccc(-c2nc3c(C(F)(F)F)cc4[nH]ncc4c3c3c2CC(N2CCOCC2)CC3)cn1]

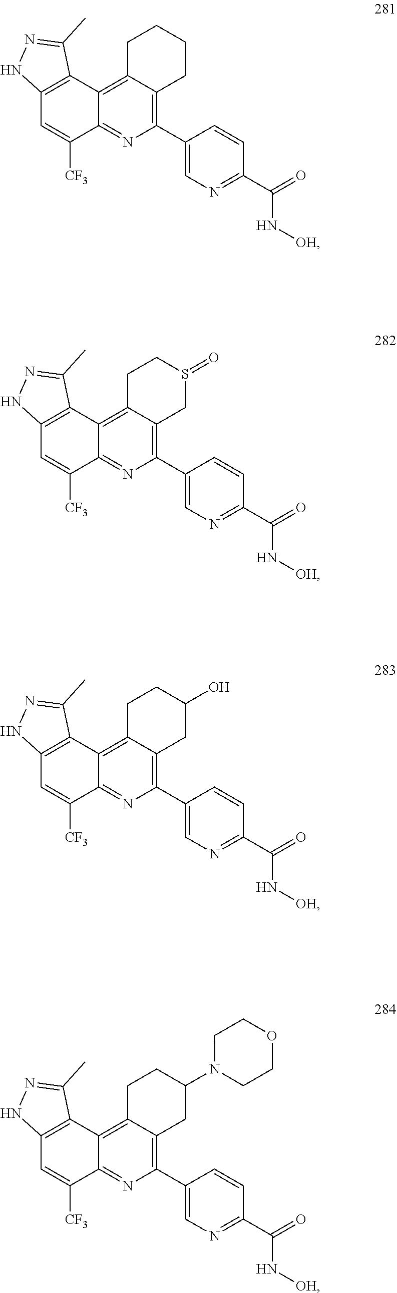 [SMILES: Cc1n[nH]c2cc(C(F)(F)F)c3nc(-c4ccc(C(=O)NO)nc4)c4c(c3c12)CCCC4 ; Cc1n[nH]c2cc(C(F)(F)F)c3nc(-c4ccc(C(=O)NO)nc4)c4c(c3c12)CCS(=O)C4 ; Cc1n[nH]c2cc(C(F)(F)F)c3nc(-c4ccc(C(=O)NO)nc4)c4c(c3c12)CCC(O)C4 ; Cc1n[nH]c2cc(C(F)(F)F)c3nc(-c4ccc(C(=O)NO)nc4)c4c(c3c12)CCC(N1CCOCC1)C4]

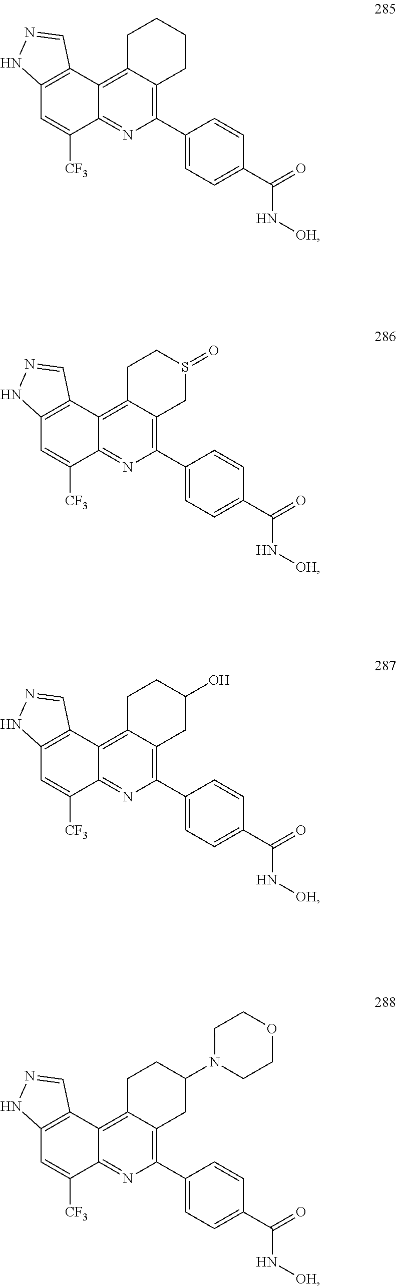 [SMILES: O=C(NO)c1ccc(-c2nc3c(C(F)(F)F)cc4[nH]ncc4c3c3c2CCCC3)cc1 ; O=C(NO)c1ccc(-c2nc3c(C(F)(F)F)cc4[nH]ncc4c3c3c2CS(=O)CC3)cc1 ; O=C(NO)c1ccc(-c2nc3c(C(F)(F)F)cc4[nH]ncc4c3c3c2CC(O)CC3)cc1 ; O=C(NO)c1ccc(-c2nc3c(C(F)(F)F)cc4[nH]ncc4c3c3c2CC(N2CCOCC2)CC3)cc1]

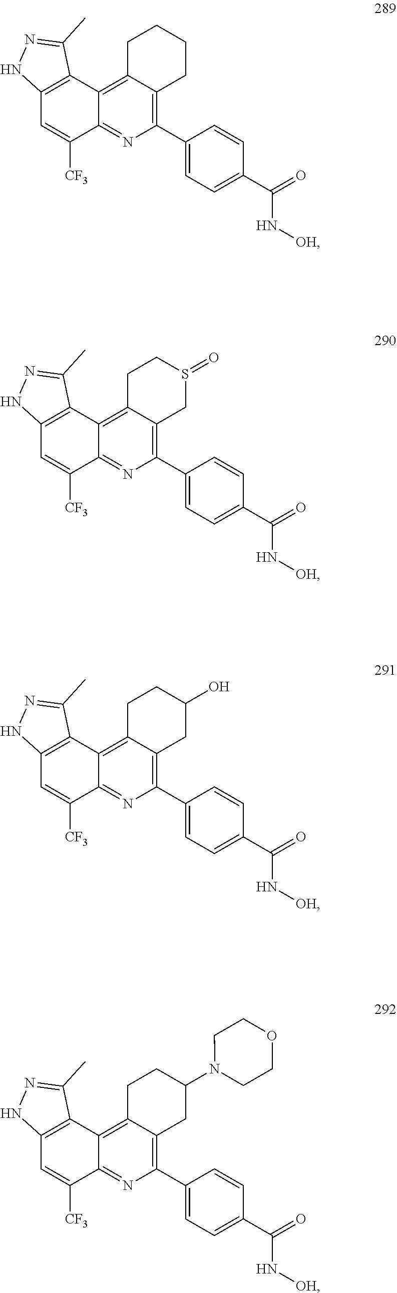 [SMILES: Cc1n[nH]c2cc(C(F)(F)F)c3nc(-c4ccc(C(=O)NO)cc4)c4c(c3c12)CCCC4 ; Cc1n[nH]c2cc(C(F)(F)F)c3nc(-c4ccc(C(=O)NO)cc4)c4c(c3c12)CCS(=O)C4 ; Cc1n[nH]c2cc(C(F)(F)F)c3nc(-c4ccc(C(=O)NO)cc4)c4c(c3c12)CCC(O)C4 ; Cc1n[nH]c2cc(C(F)(F)F)c3nc(-c4ccc(C(=O)NO)cc4)c4c(c3c12)CCC(N1CCOCC1)C4]

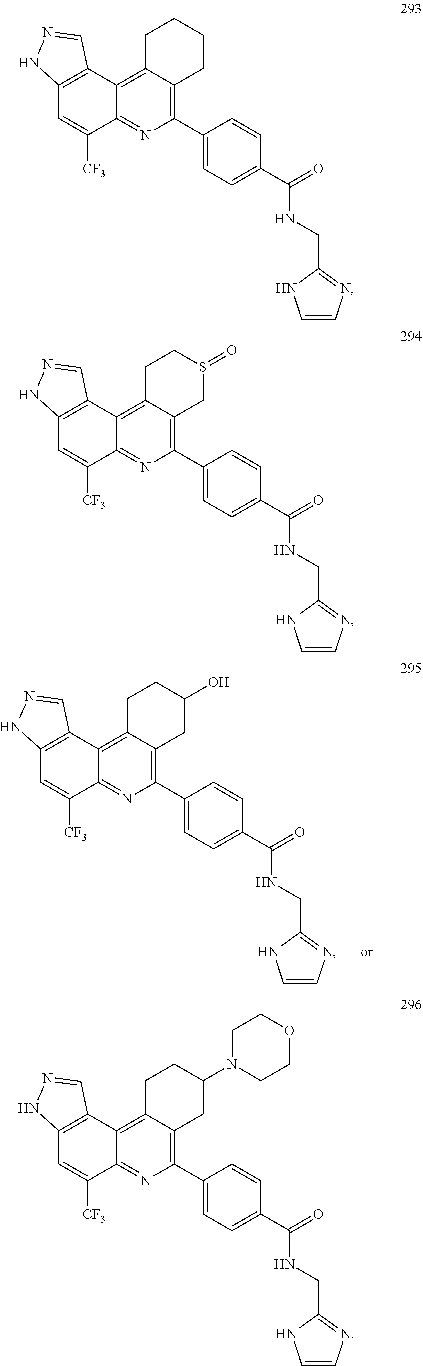

29-31. (canceled)

32. A pharmaceutical composition comprising:
a compound of claim 1, or a pharmaceutically acceptable salt, N-oxide, hydrate, solvent, tautomer, or optical isomer of the compound; and
a pharmaceutically acceptable carrier and/or diluent.

33. (canceled)

34. A method of treating cancer in a subject, the method comprising administering to the subject a therapeutically effective amount of:
a compound of claim 1, a conjugate of claim 29, or a pharmaceutically acceptable salt, N-oxide, hydrate, solvate, tautomer, or optical isomer of the compound; or
a pharmaceutical composition comprising the compound or a pharmaceutically acceptable salt, N-oxide, hydrate, solvate, tautomer, or optical isomer of the compound.

35-37. (canceled)

38. The method of claim 34, wherein the cancer is acute myeloid leukemia (AML).

39-44. (canceled)
